(19) United States
(12) Patent Application Publication
(10) Pub. No.: US 2019/0256933 A1
(43) Pub. Date: Aug. 22, 2019

(54) Guayule Molecular Markers And Primers And Use Of Molecular Markers, Primers And Rate Of Apomixis For Guayule Identification, Characterization And Breeding

(71) Applicants: Vonmark V. CRUZ, Eloy, AZ (US); David A. DIERIG, Eloy, AZ (US); Bridgestone Corporation, Chuo-ku (JP)

(72) Inventors: Vonmark V. Cruz, Casa Grande, AZ (US); David A. Dierig, Phoenix, AZ (US)

(73) Assignee: Bridgestone Corporation, Chuo-ku (JP)

(21) Appl. No.: 15/768,661

(22) PCT Filed: Oct. 14, 2016
(86) PCT No.: PCT/US2016/057227

§ 371 Date: Apr. 16, 2018

Related U.S. Application Data
(60) Provisional application No. 62/242,951, filed on Oct. 16, 2015.
(60) Provisional application No. 62/242,960, filed on Oct. 16, 2015.
(60) Provisional application No. 62/399,993, filed on Sep. 26, 2016.

(51) Int. Cl. C12Q 1/6895; A01H 6/14; C08L 7/00

(52) U.S. Cl. CPC C12Q 1/6895; A01H 6/14; C12Q 2600/158; C12Q 2600/13; C12Q 2600/156; C08L 7/00

(57) ABSTRACT

The present invention is directed to guayule molecular markers and primers and use of those markers and primers for guayule identity, characterization and breeding. The invention is further directed to a method for breeding guayule that involves determining the rate of apomixis of one or more guayule plants, determining the genetic pedigree of one or more guayule plants, and using that information to identify one or more select guayule plants for use in guayule plant breeding to produce an advanced guayule plant.

### CROSS-REFERENCE TO RELATED APPLICATIONS

This application claims the benefit of U.S. Provisional Application Ser. Nos. 62/242,951 and 62/242,960, filed Oct. 16, 2015, and U.S. Provisional Patent Application Ser. No. 62/399,993, filed Sep. 26, 2016, all of which are incorporated herein by reference in their entirety.

### STATEMENT OF FEDERALLY FUNDED RESEARCH

Not applicable.

### TECHNICAL FIELD OF THE INVENTION

The present invention relates in general to the fields of guayule plants, molecular markers and primers, the rate of apomixis and plant identification, characterization and breeding techniques using such molecular markers, primers and rates of apomixis.

### BACKGROUND ART

Natural rubber is a valuable and strategically important biomaterial. The predominant source of natural rubber cultivated for commercial production is Hevea brasiliensis, which is grown mostly in South America, Southeast Asia and areas of Africa and India. To meet the demands for natural rubber and provide a sustainable source of biorubber in alternative geographic locations, other species of rubber producing plants have been studied.

Guayule (Parthenium argentatum) is a perennial desert shrub species native to northcentral Mexico and southwestern Texas. Guayule is being studied as an alternative source of natural rubber for potential large scale commercial production. Guayule produces rubber within its cells that generally must be destructively harvested after the plant reaches maturity. The guayule produced rubber also provides a benefit of being free from the proteins responsible for latex allergies that can be caused by Hevea sourced natural rubber.

There is a need for diversifying the production of natural rubber and improvements in the breeding and characteristics of guayule plants that produce natural rubber.

### SUMMARY OF THE INVENTION

The present invention is directed to molecular markers for guayule and methods of using those molecular markers to identify, characterize and breed guayule. In a preferred embodiment, the molecular markers are single nucleotide polymorphisms (SNPs) or short tandem repeating mono-, di-, tri-, tetra- penta, and hexanucleotide microsatellites (simple sequence repeats or SSRs). Further preferably, the molecular markers are polymorphic. In another preferred embodiment the molecular markers are associated with expressed traits of the guayule plant, particularly those associated with improved plant yield, size, uniformity and rubber production. The invention is further directed to methods for determining the rate of apomixis in guayule plants and using information regarding the rate of apomixis in a method for breeding a guayule plant, for example, in the identification of guayule plants for breeding, either alone or in combination with a step of determining the genetic pedigree of a plant. e.g., by DNA analysis using SNPs and/or SSRs.

In another embodiment, the present invention is directed to primers associated with SNPs or SSRs according to the invention. In a preferred embodiment, the primers are used in a method to amplify a molecular marker such as an SNP or SSR, and in a further preferred embodiment that molecular marker is polymorphic. In a further preferred embodiment, the invention is directed to a primer kit containing a set of primers to be used in the methods according to the invention.

In one embodiment, the molecular markers and primers according to the invention are used in a method to evaluate the genetic diversity of guayule germplasm from one or more collections of guayule germplasm.

In another embodiment, the molecular markers and primers according to the invention are used in a method to quantify genetic similarity or distance among accessions within one or more collections of guayule germplasm.

In another embodiment, the molecular markers and primers according to the invention are used in a method of parenteral germplasm screening and/or selection.

In another embodiment, the molecular markers and primers according to the invention are used in a method for the genetic advancement of guayule germplasm.

In another embodiment, the molecular markers and primers according to the invention are used in a method to screen and select one or more guayule cells, tissues, seedlings, plants, plant parts, hybrids or progeny thereof.

In another embodiment, the molecular markers and primers according to the invention are used in a method to develop new varieties of guayule.

In another embodiment, the molecular markers and primers according to the invention are used in a method of selection for one or more traits of guayule cells, tissues, seedlings, plants, plant parts, hybrids or progeny thereof.

In another embodiment, the molecular markers and primers according to the invention are used in a method of identification, validation and/or quality assessment, including germplasm identity, parental background recovery, and seed purity testing.

In a further preferred embodiment, the molecular markers, primers and methods according to the invention are used to identify or improve an expressed trait of a guayule plant including rubber content, rubber quality, rubber yield, average molecular weight of rubber, plant size, plant yield, plant vigor, plant uniformity, salinity tolerance, temperature tolerance, disease resistance, or drought tolerance.

In a further preferred embodiment, the invention is directed to guayule plant cells, plant tissue, seedlings, plant parts, plants, and progeny thereof produced by any of the methods according to the invention. Another embodiment of the invention is the rubber composition produced by or extracted from such guayule plants produced by any of the methods according to the invention. In one embodiment, those guayule plant cells, plant tissue, seedlings, plant parts, plants, and progeny thereof have an improved expressed trait, phenotype and/or genotype over naturally occurring guayule or publicly available guayule germplasm.

The invention is also directed to products comprising the rubber produced by or extracted from guayule plants according to the invention or in plants produced by any of the methods according to the invention. Those products may include tires and latex products for the medical profession.

### DESCRIPTION OF THE DRAWINGS

For a more complete understanding of the features and advantages of the present invention, reference is now made to the detailed description of the invention along with the accompanying figures.

FIG. 1 is a principal coordinate analysis of guayule accessions showing the 2D relationships among the represented guayule germplasm per ploidy level.

FIG. 2 is a principal coordinate analysis using pairwise genetic distances among individual plant samples from 54 SSR markers.

FIG. 3 is a diagram of Bayesian cluster assignments of guayule germplasm from controlled crosses.

FIG. 4 is a tree representing the genetic relationships among a subset of private and public guayule germplasm (red=private; black=public germplasm) using SSR markers.

FIG. 5 is a general outline of NGS assisted plant breeding from Ray, S. and Sataya, P., 2014 “Next Generation Sequencing Technologies for Next Generation Plant Breeding,” Front. Plant Sci.

FIG. 6 is a diagram providing an overview of paired-end RAD sequencing. In section A, genomic DNA is digested with a restriction endonuclease. In section B, after ligation with a primary adapter, the fragments are sheared, then ligated with a secondary adapter. In section C, a composite mixture of variable length fragments is recovered from each restriction enzyme digestion site. These fragments are size selected, amplified and sequenced on a next-generation DNA sequencing platform using paired end chemistry. In section D, development of the genomic assembly around each digestion site is then completed bioinformatically.

FIG. 7 is a DNA quality check of four guayule DNA samples.

FIG. 8 is a diagram of a summary neighbor joining tree from Nei's distances showing the relationships among the different diploid HS and USDA germplasm, BART historical germplasm, and other representative polyploids such as G7-11 (Rec ID 88716 and 88717).

FIG. 9 is a diagram of a tree showing genetic similarity among individual plants of each germplasm type (HS or accession). Colors denote original group assignment. Some clusters contain plants from one or more HS (e.g. HS 5 and HS 14) indicating genetic admixture. Polyploid germplasm are shown to separately group from diploids.

FIG. 10 is a diagram of expected heterozygosity of SNP loci within each diploid germplasm.

FIG. 11 is a matrix of average number of pairwise differences among the diploid germplasm. The above diagonal indicates differences between germplasm, the diagonal elements show differences within germplasm, and the below diagonal the Nei pairwise genetic distances.

FIG. 12 is a diagram of the possible reproductive classes from a tetraploid X tetraploid.

FIG. 13 is a graph of seed maturity vs. nuclei staining (red=seed; black=2x reference).

FIG. 14 is a graph of embryo ploidy detectable at all development stages (red=early; blue=mid; late=black).

FIG. 15 is a graph of DNA content—seed+internal control.

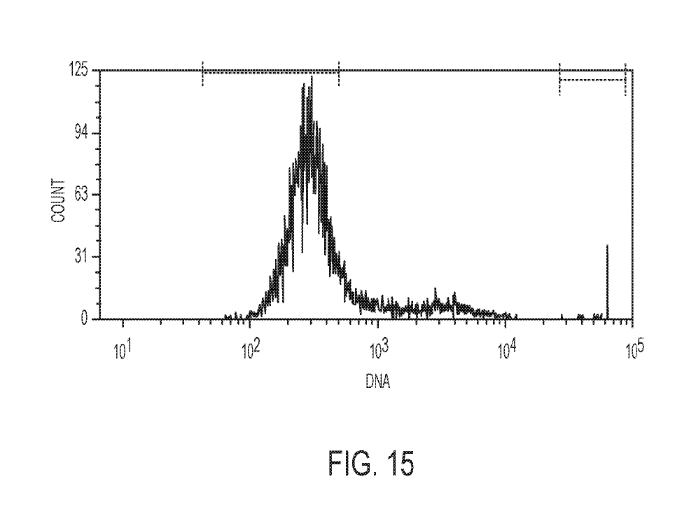

FIG. 16 is a graph of apomictic seed from a 4x plant.

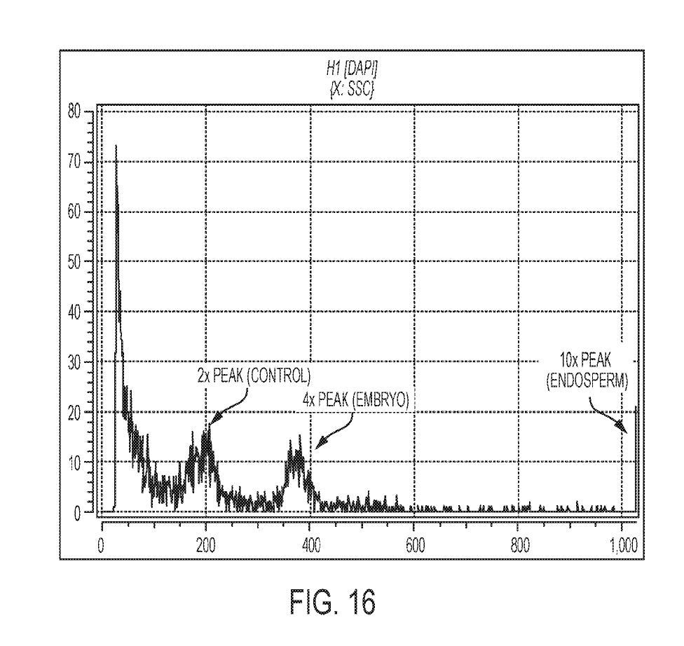

### DETAILED DESCRIPTION OF THE INVENTION

The present invention is directed to the genetic improvement of guayule. In a preferred embodiment, the compositions and methods according to the present invention can be used to improve a guayule crop for higher plant yield, size, uniformity and rubber production, and other traits that will make it suitable for large scale agriculture.

The present invention is directed to molecular markers for guayule and methods of using those molecular markers to identify, characterize and breed guayule. In a preferred embodiment, the molecular markers are single nucleotide polymorphisms (SNPs) or short tandem repeating mono-, di-, tri-, tetra- penta, and hexanucleotide microsatellites (simple sequence repeats or SSRs). Further preferably, the molecular markers are polymorphic. In a further preferred embodiment, the SNPs are selected from the SNPs listed in Table 9. In another preferred embodiment, the SSRs are selected from the SSRs listed in Tables 11 and 12.

In another embodiment, the present invention is directed to the primers associated with SNPs or SSRs according to the invention. In a preferred embodiment, the primers are used in a method to amplify a molecular marker such as an SNP or SSR, and in a further preferred embodiment that molecular marker is polymorphic. In another embodiment, the invention is directed to an isolated nucleic acid molecule, or primer, for detecting a molecular marker representing a polymorphism in guayule plant DNA, wherein said nucleic acid molecule comprises at least 15 nucleotides that include or are immediately adjacent to said polymorphism, wherein said nucleic acid molecule is at least 90 percent identical to a sequence of the same number of consecutive nucleotides in either strand of DNA that include or are immediately adjacent to said polymorphism. The polymorphism is preferably a polymorphic SNP or SSR, for example, a SNP or SSR identified in Tables 9, 11 and 12. In a further preferred embodiment, the invention is directed to a primer kit containing a set of primers to be used in the methods according to the invention. In a preferred embodiment, the set of primers comprises one or more primers listed in Tables 11 and 12.

In one embodiment, the molecular markers and primers according to the invention are used in a method to evaluate the genetic diversity of guayule germplasm from one or more collections of guayule germplasm. For example, the invention may be directed to a method for determining the contributions of guayule populations to a guayule genome, comprising performing a genotyping assay on a sample obtained from a test guayule plant to determine the identity of one or both alleles present in the test guayule plant genome for each set of markers, wherein the set of markers is indicative of the contribution of guayule populations of the genome of the test guayule plant. In a preferred embodiment, the set of markers used in that method comprises at least five markers or, more preferably, at least ten markers. In one embodiment, the set of markers are selected from single nucleotide polymorphisms (SNPs) or single nucleotide repeat microsatellites, di-, tri-, tetra-, penta- and hexanucleotide simple sequence repeats (SSRs). In a further preferred embodiment, the set of molecular markers comprises markers listed in Tables 9, 11 and 12.

In another embodiment, the molecular markers and primers according to the invention are used in a method to quantify genetic similarity or distance among accessions within one or more collections of guayule germplasm.

In another embodiment, the molecular markers and primers according to the invention are used in a method of parenteral germplasm screening and/or selection. For example, a preferred embodiment is directed to a method for determining the genotype of guayule germplasm, comprising the steps of (a) amplifying the guayule germplasm using a set of primers comprising primers identified in Tables 11 and 12; and (b) determining the genotype using a combination of molecular markers comprising molecular markers identified in Tables 11 and 12. Another embodiment of the invention is a primer kit comprising primers identified in Tables 11 and 12.

In another embodiment, the molecular markers and primers according to the invention are used in a method for the genetic advancement of guayule germplasm.

In another embodiment, the molecular markers and primers according to the invention are used in a method to screen and select one or more guayule cells, tissues, seedlings, plants, plant parts, hybrids or progeny thereof.

In another embodiment, the molecular markers and primers according to the invention are used in a method to develop a new variety of guayule. For example, the invention may be directed to a guayule plant breeding method comprising (a) evaluating guayule plants using objective criteria to obtain phenotype scores; (b) determining the genetic pedigree of the guayule plants using DNA analysis; and (c) using the phenotype scores and genetic pedigrees to identify one or more select guayule plants for use in a next generation of guayule plant breeding to produce an advanced generation of guayule plants. Preferably, the DNA analysis is performed using DNA of single nucleotide polymorphism (SNP) or single nucleotide repeat microsatellites, di-, tri-, tetra-, penta-, or hexanucleotide simple sequence repeats (SSRs). In a further preferred embodiment, two select guayule plants are identified that are genetically dissimilar or highly dissimilar and the two select plants are crossed to produce an advanced generation of guayule plants.

In another embodiment, the molecular markers and primers according to the invention are used in a method of selection for one or more traits of guayule cells, tissues, seedlings, plants, plant parts, hybrids or progeny thereof. For example, one embodiment of the invention is directed to a method for identifying a genetic marker linked to a trait locus of a guayule plant, said method comprising the steps of: (a) extracting nucleic acid from a guayule plant; (b) amplifying the nucleic acid using a set of primers comprising primers identified in Tables 11 and 12; (c) determining the genotype using a set of molecular markers comprising molecular markers identified in Tables 11 and 12; and (d) associating a genetic marker with an expressed trait of the guayule plant. In a further preferred embodiment, the genetic marker is linked to a trait such as rubber content, rubber quality, rubber yield, average molecular weight of rubber, plant size, plant vigor, plant uniformity, salinity tolerance, temperature tolerance, disease resistance, or drought tolerance. In a preferred embodiment, the temperature tolerance is cold tolerance.

In another embodiment, the molecular markers and primers according to the invention are used in a method of identification, validation and/or quality assessment, including germplasm identity, parental background recovery, and seed purity testing.

In a further preferred embodiment, the molecular markers, primers and methods according to the invention are used to improve the characteristics of a guayule plant including rubber content, rubber quality, rubber yield, average molecular weight of rubber, plant size, plant vigor, plant uniformity, salinity tolerance, temperature tolerance, disease resistance, or drought tolerance.

In a further preferred embodiment, the invention is directed to guayule plant cells, plant tissue, seedlings, plant parts, plants, and progeny thereof produced by any of the methods according to the invention. Another embodiment of the invention is the rubber composition produced by or extracted from such guayule plants produced by any of the methods according to the invention. In one embodiment, those guayule plant cells, plant tissue, seedlings, plant parts, plants, and progeny thereof have an improved expressed trait, phenotype and/or genotype over naturally occurring guayule or publicly available guayule germplasm.

The invention is also directed to products comprising the rubber produced by or extracted from plants according to the invention, including tires and latex products for the medical profession.

### Experimental Procedures

A study was conducted to develop microsatellite (SSR) and single nucleotide repeat (SNP) markers that can be used in methods according to the invention, including methods in which molecular markers are used to screen for genetic diversity, perform marker assisted selection, and aid trait introgression for guayule breeding.

Publicly available guayule expressed sequenced tags were used to develop 713 SSR primer pairs and testing was completed on 193 SSRs. A set of SSRs was used to screen the genetic diversity of USDA guayule collection and a private germplasm collection to assess the genetic variation within accessions and among various guayule genetic groups.

In addition to SSRs, SNP markers were developed for guayule breeding. A total of 1,065 SNPs were identified from four guayule sequence libraries and the level of polymorphism was studied. SNPs are identified in Table 9. SSRs and their corresponding primers are identified in Tables 11 and 12. The two marker systems provide unique opportunities for use in guayule molecular breeding for plant improvement.

Molecular markers for guayule were developed to increase the available information for decision making during guayule improvement activities. Research to find markers that are useful for estimating genetic diversity also aimed to identify molecular markers that are tightly linked to traits. Having these useful markers will enable rapid screening of segregating phenotypes at an early stage of development, with potential time and cost savings.

A. Development and Testing of Microsatellite Markers (SSR)

Initial work to develop primers for microsatellite (simple sequence repeat or SSR) markers was completed. Expressed sequenced tags (EST) made publicly available by the USDA-ARS Elastomics Project through the NCBI EST online database were downloaded. A total of 11.739 EST sequences were obtained and contigs assembled by using an overlap identity cutoff of 80% and using masking processes to identify repeats, vector, and organelle sequences. From the contigs, 337 microsatellite units were identified while 515 microsatellite units on the singletons (sequences did not contain enough overlap in length to be combined with others). The microsatellites from the contigs have the following proportion of repeat units: 73 (21.7%) di-, 242 (71.8%) tri-, 12 (3.5%) tetra-, and 10 (3.0%) hexa-nucleotides. Primers for microsatellite units in contigs and singletons were designed based on the following parameters: 100-500 amplicon size, 18-24 bp primer size, 40-60% G-C content, 55-60° C., with 58° C. optimal Tₘ. A total of 198 SSR primers were designed from the contigs and 515 from the singletons (see Tables 11 and 12 for primer sequences and SSRs).

Preliminary primer testing was conducted using seven guayule germplasm (11591, 11604, 141660, 141678, AZ-2, S1, S3) representing various ploidy levels (2× to 6×). Leaf tissue was obtained on 10 to 12 individual plants per accession and the DNA was processed for extraction and genotyping. Two bulks of DNA samples per accession were made by pooling DNA from six different plants (five for the triploid accessions).

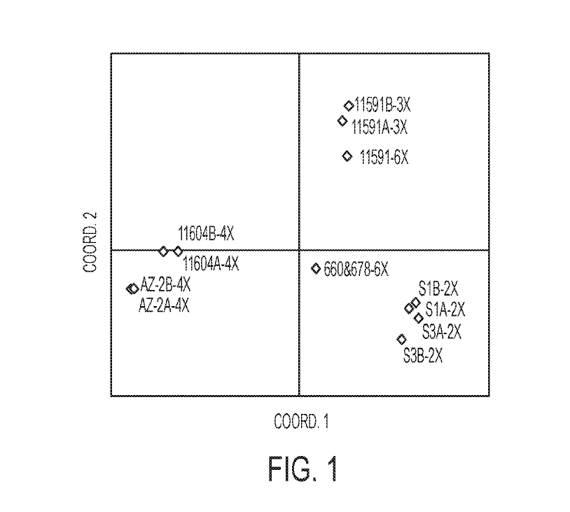

An 18 bps M13 fragment was added to forward primer of all 198 markers and FAM fluorescent dye was used to label the primers. Polymerase chain reaction (PCR) was performed using Applied Biosystems® GeneAmp® PCR System 9700 following 50 cycles with 95° C. denaturation, 55° C. annealing and 72° C. extension. The amplified products were run on ABI 3730x1 sequencer in order to separate DNA fragments based on their molecular weights. Out of 198 tested markers 20 (10%) failed to amplify any part of the genome, while 178 (90%) successfully amplified. A total of 93 markers (47%) were polymorphic while 85 primers (43%) were monomorphic. The genetic distances among accessions were computed following the method of Huff et al. (1993) treating the polymorphic marker scores as dominant since the test set included polyploid guayule germplasm. The principal coordinates plot, showing the 2D relationships among the represented guayule germplasm per ploidy level, was computed and are shown in FIG. 1.

The 93 polymorphic markers were further assessed to determine marker polymorphism using individual plant samples and determine their robustness. Two to five individual plants representing each accession were used. Out of the 93 polymorphic markers, 54 markers were found to be most promising and easy to score and can be readily utilized for analyzing the entire guayule germplasm collection. An additional 18 markers were also determined as useful, though these set will need further performance validation using fluorescent labels. The other 21 markers were found to be difficult to score but may have utility in the long term.

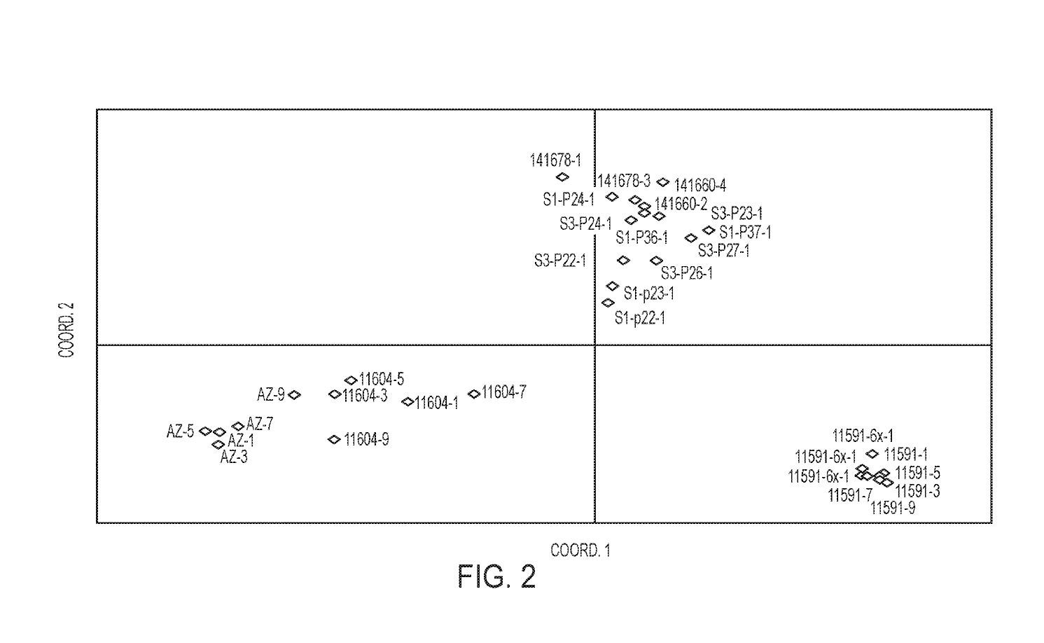

The genetic distances among individual plant samples were determined from results of using the 54 markers. The principal coordinates plot is shown in FIG. 2. The plot shows the genetic similarity among samples of each ploidy level. The tight clustering of samples of 11591 and the low mean pairwise genetic distance (Table 1) suggest that this accession has more narrow genetic diversity than AZ-2. The diploid populations (S1 and S3) have higher mean pairwise distance values than the tetraploid accessions. These preliminary observations remain to be confirmed using larger sets of samples.

TABLE 1
Pairwise population matrix of mean population binary genetic distance

| | 11591 | 11591-6x | 11604 | 141660 | 141678 | AZ-2 | S1 | S3 |
|---|---|---|---|---|---|---|---|---|
| 11591 | 16.600 | | | | | | | |
| 11591- | 15.667 | 13.000 | | | | | | |
| 6x | | | | | | | | |
| 11604 | 65.120 | 64.267 | 39.900 | | | | | |
| 141660 | 61.900 | 57.833 | 68.000 | 56.000 | | | | |
| 141678 | 76.100 | 73.000 | 80.500 | 59.750 | 42.000 | | | |
| AZ-2 | 75.680 | 73.800 | 46.760 | 77.100 | 82.800 | 19.500 | | |
| S1 | 54.480 | 52.400 | 61.800 | 53.100 | 61.500 | 65.560 | 50.000 | |
| S3 | 52.920 | 50.400 | 60.480 | 52.900 | 56.000 | 68.840 | 49.040 | 39.600 |

Microsatellite markers have many applications in biological studies of plants, molecular genomics, and in a breeding program. They can be used to determine taxonomic classification, population structure, genetic diversity, and marker-trait associations.

First generation (F1) progenies from polyhaploid (n=36)×tetraploid (n=72) guayule germplasm along with parental materials were genotyped using microsatellite markers (SSRs) to test if possible to validate the parentage of the progenies and confirm that they arose from sexual recombination. The SSR markers used were those determined as most robust in the set (54 of 93 SSRs) during preliminary testing. Those 54 SSR markers correspond to 4, 5, 6, 8, 10, 12, 16, 18, 19, 24, 26, 37, 43, 51, 52, 58, 61, 68, 70, 71, 80, 93, 107, 108, 112, 114, 115, 118, 119, 126, 128, 132, 134, 139, 140, 143, 144, 156, 157, 158, 162, 164, 165, 170, 171, 180, 181, 183, 185, 186, 188, 195, 197, and 198 in Table 11.

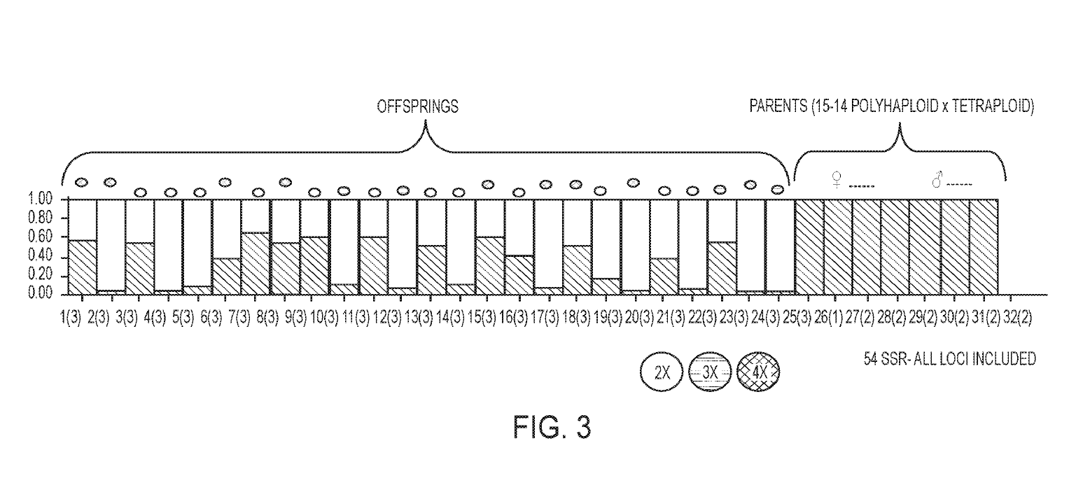

The Bayesian clustering results using the marker data provided additional information about possible paternity and parental mode of reproduction. The cluster assignments of the genotyped germplasm are shown in FIG. 3. This method is originally intended for determining population structure but also adapted for this purpose.

Leaf tissues from 140 guayule germplasm were collected for SSR genotyping (Table 2). A subset of 15 guayule SSRs was used to determine the genetic similarity among breeding materials and with the USDA germplasm collection to assist the breeding program. Those 15 SSR markers correspond to 4, 5, 10, 12, 18, 19, 68, 80, 93, 134, 139, 158, 185, 186 and 188 in Table 11.

For genotyping, the M13-tailed SSR primers used during the primer testing phase were replaced by fluorescent-labeled primers which are more stable, reliable than M13-labelled primers, and gave reproducible results needed for routine genotyping.

TABLE 2
List of guayule accessions in the private and
USDA collection genotyped with SSR markers

| ID | No. samples |
|---|---|
| 11591 | 5 |
| 11600 | 1 |
| 11604 | 7 |
| 11605 | 2 |
| 11619 | 1 |
| 11634 | 4 |
| 11635 | 7 |
| 141018 | 2 |
| 141209 | 1 |
| 141298 | 1 |
| 141630 | 1 |
| 141632A | 3 |
| 141634 | 1 |
| 141635 | 1 |
| 141635A | 1 |
| 141637 | 1 |
| 141652 | 1 |
| 141699 | 1 |
| 141727 | 1 |
| 141734 | 1 |
| 141735 | 1 |
| 14M-1 | 5 |
| 14M-7 | 5 |
| 4265-X | 1 |
| 4265-XF | 5 |
| 593 | 2 |
| 8012 | 1 |
| 8062 | 1 |
| 8150 | 1 |
| 8267 | 1 |
| 8269 | 1 |
| 8272 | 1 |
| 8274 | 1 |
| 8275 | 1 |
| A48118 | 1 |
| AZ-1 | 5 |
| AZ-2 | 2 |
| AZ-3 | 2 |
| AZ-4 | 1 |
| AZ-5 | 4 |
| AZ-6 | 5 |
| CAL-1 | 1 |
| CAL-2 | 1 |
| CAL-3 | 5 |
| CAL-4 | 1 |
| CAL-5 | 3 |
| CAL-6 | 5 |
| CFS11-2005 | 1 |
| MAC31-AZ2 | 5 |
| MAC31-Sel1 | 5 |
| N575 | 4 |
| R1092 | 1 |
| R1096 | 1 |
| R1100 | 4 |
| R1101 | 3 |
| R1108 | 3 |
| R1110 | 4 |
| W6-429 | 3 |

Analysis of SSR data showed that the mean major allele frequency for the marker set was 0.3752 and the polymorphism information content (PIC) was 0.7309 indicating that the marker set is highly informative. Pairwise comparison of all the germplasm using Dice coefficient (Dice, 1945) has a mean of 0.3938, with values ranging from 0.0000 to 0.7857 (9,730 total pairwise values). The highly similar germplasm, as well as extremely dissimilar germplasm pairs are listed in Table 3.

The mean pairwise genetic distance among selected private germplasm and USDA germplasm was computed following the method of Huff et al. (1993) on dominant markers and the results shown in Table 4.

TABLE 3
Pairs of guayule germplasm with extreme dissimilarity values.
(Sample IDs are noted as i and j, while the pairwise Dice
dissimilarity value as d(i, j))

| | | | | |
|---|---|---|---|---|
| i | Accession ID | j | Accession ID | d(i, j) |

Germplasm samples that are very genetically similar

| | | | | |
|---|---|---|---|---|
| 10 | MAC31-AZ2-19 | 9 | MAC31-AZ2-16 | 0.0000 |
| 34 | CAL-5 | 33 | CAL-5 | 0.0000 |
| 46 | 4265-XF | 45 | 4265-XF | 0.0000 |
| 50 | 8274 | 49 | 8267 | 0.0000 |
| 66 | 11635 | 65 | 11635 | 0.0000 |
| 69 | R1096 | 21 | AZ-6 | 0.0000 |
| 88 | 593 | 84 | N575 | 0.0000 |
| 98 | 11591 | 94 | 11591 | 0.0000 |

Germplasm samples that are very dissimilar

| | | | | |
|---|---|---|---|---|
| 118 | 14M-1-13 (2X) | 75 | R1101 | 0.7436 |
| 118 | 14M-1-13 (2X) | 3 | AZ-1 | 0.7447 |
| 118 | 14M-1-13 (2X) | 40 | CAL-6 | 0.7447 |
| 29 | CAL-3 (2X) | 1 | AZ-1 | 0.7500 |
| 118 | 14M-1-13 (2X) | 55 | 11604 | 0.7619 |
| 140 | 141632A-4 (2X) | 112 | MAC31-Sel1-31 (3X) | 0.7619 |
| 118 | 14M-1-13 (2X) | 89 | 593 | 0.7674 |
| 131 | 8012 (P) | 118 | 14M-1-13 (2X) | 0.7838 |
| 140 | 141632A-4 (2X) | 118 | 14M-1-13 (2X) | 0.7857 |

TABLE 4
Mean pairwise genetic distance among selected USDA germplasm and private
guayule selections computed by comparing up to five plants per accession

| | | | MAC31- | | | | | | | | | MAC31- |
|---|---|---|---|---|---|---|---|---|---|---|---|---|
| | AZ-1 | AZ-2 | AZ2 | AZ-3 | AZ-4 | AZ-5 | AZ-6 | CAL-3 | CAL-6 | 11591 | W6-429 | Sel1 |
| AZ-1 | 24.60 | 21.00 | 25.20 | 30.00 | 21.00 | 23.60 | 22.52 | 34.68 | 28.48 | 34.16 | 29.13 | 28.72 |
| AZ-2 | | 12.00 | 14.40 | 18.00 | 9.00 | 19.50 | 20.20 | 35.60 | 28.30 | 32.60 | 29.50 | 24.90 |
| MAC31-AZ2 | | | 17.60 | 18.20 | 11.80 | 23.40 | 22.60 | 37.88 | 31.88 | 35.20 | 33.87 | 29.92 |
| AZ-3 | | | | 20.00 | 14.00 | 24.38 | 26.00 | 37.00 | 32.90 | 37.60 | 35.50 | 32.80 |
| AZ-4 | | | | | | 17.00 | 15.60 | 30.40 | 23.20 | 28.60 | 24.33 | 21.60 |
| AZ-5 | | | | | | 21.33 | 17.65 | 34.60 | 26.00 | 31.85 | 29.00 | 24.95 |
| AZ-6 | | | | | | | 17.30 | 33.08 | 24.36 | 29.68 | 26.60 | 23.12 |
| CAL-3 | | | | | | | | 18.30 | 27.88 | 27.28 | 17.80 | 29.28 |
| CAL-6 | | | | | | | | | 20.70 | 30.20 | 25.00 | 26.72 |
| 11591 | | | | | | | | | | 12.50 | 20.53 | 21.76 |
| W6-429 | | | | | | | | | | | 6.67 | 23.33 |

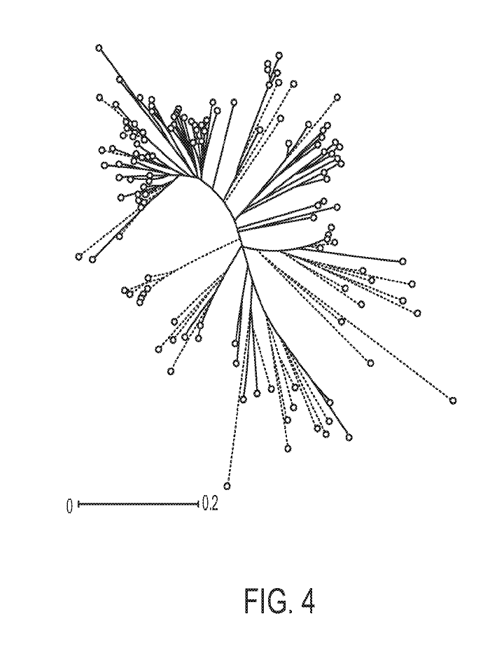

A tree representing the genetic similarity among the different accessions in the private and public collections using 15 SSR markers is shown in FIG. 4.

The SSRs will be useful in establishing a genetic linkage map of guayule and identification of quantitative trait loci (QTL). This can be the basis to develop mapping populations to determine the distribution of the SSR markers in various linkage groups the guayule. Both SSRs and SNPs will be used for estimating recombination events, establishing marker-trait association in quantitative and qualitative traits of guayule.

SSR markers may be used to anchor next-generation genome sequencing data into chromosomes. Contigs where markers were detected may be used to physically map genetic marker sequences and assess their location on the genetic map.

Information from SSR and SNPs can be used to establish genotype information for future proprietary guayule varieties. The information from both molecular marker systems will allow identity and purity testing of seed supply as well as gene flow or potential contamination.

B. Development and Testing of SNPs (RAD-Seq) Markers

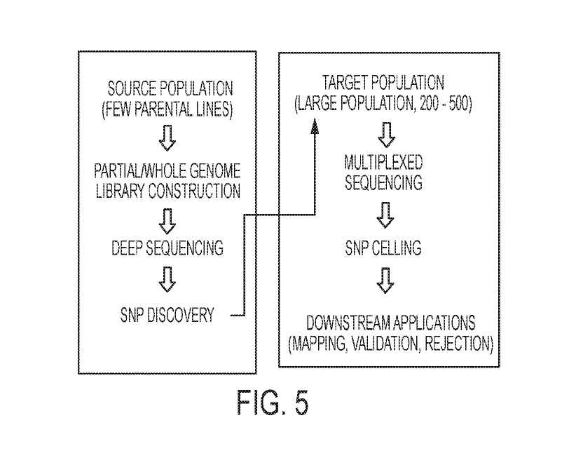

Restriction-site associated DNA sequencing (RAD-seq) as a next generation sequencing (NGS) technology platform was employed to obtain large numbers of SNP markers. The potential of NGS technologies for marker assisted breeding have been reviewed in the literature and FIG. 5 is an example from Ray, S., and Sataya. P., 2014 “Next Generation Sequencing Technologies for Next Generation Plant Breeding.” Front. Plant Sci.

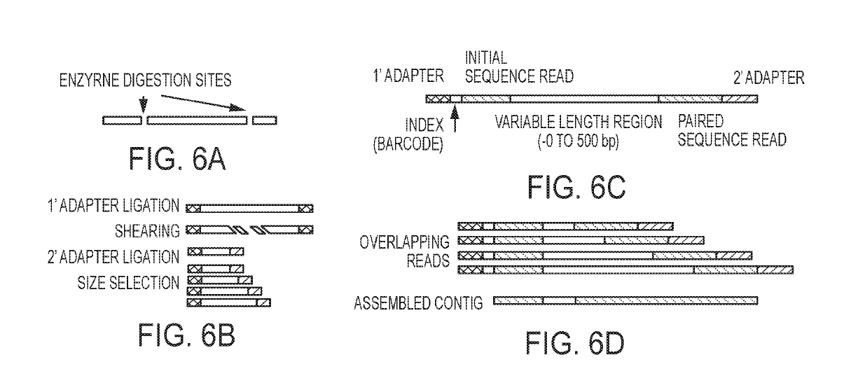

Four individual leaf samples of 4 accessions were received for SNP discovery project. Discovery of SNPs can be effectively performed by using next-generation sequencing (NGS) technologies. NGS technologies and the use of a complexity reduction strategy can successfully be applied for SNP discovery in large and complex genomes with no reference genome. Restriction site-associated DNA sequencing (RAD-Seq) is commonly used for SNP detection in genomes and is based on identifying polymorphism next to restriction enzyme digestion sites. Paired-end RAD sequencing was applied for detection of sequence variation and design of SNP markers. An overview of pair-end RAD sequencing is shown in FIG. 6. In section A, genomic DNA is digested with a restriction endonuclease. In section B, after ligation with a primary adapter, the fragments are sheared, then ligated with a secondary adapter. In section C, a composite mixture of variable length fragments is recovered from each restriction enzyme digestion site. These fragments are size selected, amplified and sequenced on a next-generation DNA sequencing platform using paired end chemistry. In section D, development of the genomic assembly around each digestion site is then completed bioinformatically.

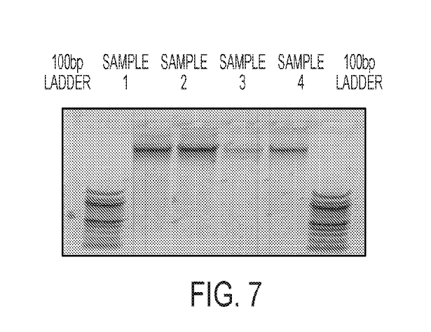

To develop the RAD genotyping platform for guayule, leaf tissue samples were collected from 11591, 8150 (Cal 3), 141632A (W6-429), and 14M-1-12 (S1) germplasm to obtain 3 μg of high molecular weight DNA required for DNA library development. The DNA quality and quantity was confirmed by agarose gel as shown in FIG. 7. DNA sequencing was performed on each sample and development of RAD libraries followed. The associated statistics with the four RAD libraries are shown below. The number of sequencing reads indicates the number of short DNA fragments that have been sequenced.

Total Reads: 30,211,365

Mean Reads per Sample: 6,042,273

Median Reads per Sample: 6,488,899

Read Goal per Sample: 4,000,000

Std. Dev of Reads per Sample: 1,899,084

Number of Samples Meeting Read Goals: 4

Sequencing Coefficient of Variation: 0.31

TABLE 5

| Sample ID | NGS Reads | Read Goal | RAD Clusters |
|---|---|---|---|
| 11591 (4X) | 8,656,563 | 4,000,000 | 29,973 |
| 14M-1-12 (2X) | 6,705,768 | 4,000,000 | 24,366 |
| 141632A-3 (2X) | 6,488,899 | 4,000,000 | 20,754 |
| 8150 (2X) | 4,270,193 | 4,000,000 | 19,587 |

The sequence data for guayule was analyzed to find markers with good characteristics for downstream genotyping. There were 1,065 markers that met the required characteristics for genotyping (80 bp flanking sequence, no other markers within 50 bp). Those SNP molecular markers are shown in Table 9. A total of 970 primers were designed and tested for marker polymorphism and informativeness.

For testing, leaf tissues were harvested from five plants of 12 diploid accessions (14M-7, CAL-3, HS10, HS12, HS14, HS15, HS17, HS18, HS2, HS5, HS6, and W6-429) and genotyped. Two sets of primers were used for the initial screening of the diploid accessions. The first set of 384 primers indicate that 379 SNPs successfully amplified, 5 failed, and 3.6% has missing data. Of the 384 markers, 319 are polymorphic while 60 were monomorphic. The mean allele frequency was 0.65, the PIC was 0.28 and mean heterozygosity was 0.68. The genetic distances among the 12 accessions were estimated using the method of Nei (1972) (Table 6).

TABLE 6
Genetic distances among guayule diploid
accessions using 379 SNP markers

| OUT | 14M-7 | CAL-3 | HS10 | HS12 | HS14 | HS15 | HS17 | HS18 | HS2 | HS5 | HS6 | W6-429 |
|---|---|---|---|---|---|---|---|---|---|---|---|---|
| 14M-7 | 0.000 | | | | | | | | | | | |
| CAL-3 | 0.058 | 0.000 | | | | | | | | | | |
| HS10 | 0.068 | 0.070 | 0.000 | | | | | | | | | |
| HS12 | 0.068 | 0.066 | 0.0.32 | 0.000 | | | | | | | | |
| HS14 | 0.068 | 0.065 | 0.039 | 0.027 | 0.000 | | | | | | | |
| HS15 | 0.065 | 0.060 | 0.057 | 0.038 | 0.044 | 0.000 | | | | | | |
| HS17 | 0.071 | 0.068 | 0.039 | 0.022 | 0.039 | 0.047 | 0.000 | | | | | |
| HS18 | 0.066 | 0.050 | 0.044 | 0.034 | 0.036 | 0.035 | 0.047 | 0.000 | | | | |
| HS2 | 0.083 | 0.080 | 0.045 | 0.041 | 0.039 | 0.065 | 0.041 | 0.057 | 0.000 | | | |
| HS5 | 0.064 | 0.065 | 0.058 | 0.041 | 0.045 | 0.029 | 0.047 | 0.031 | 0.074 | 0.000 | | |
| HS6 | 0.074 | 0.063 | 0.035 | 0.033 | 0.035 | 0.034 | 0.032 | 0.042 | 0.056 | 0.026 | 0.000 | |
| W6-429 | 0.079 | 0.075 | 0.066 | 0.061 | 0.051 | 0.072 | 0.062 | 0.062 | 0.060 | 0.078 | 0.060 | 0.000 |

TABLE 7
Examples of SNP sequences discovered by RAD-Seq method

| #CHROM | POS | SEQ |
|---|---|---|
| RAD_kmer_0000025_1_TGC [symbol] | 107 | CGCCACACATAAATAATCAAACCTTATCCGTCACCACTTGAGAAAAACCCTATACA |
| | | GGGCGGCATGCCGCCACTGTCGCT[A/G]CTGCCCTGTCCGGCCGCCATTTGTTTTTG |
| | | TTTTCTGTTTCGTCTTTCTTGTTTCCTGTTCTAAAAAGACTAGGTCTAGGT |
| RAD_kmer_0000028_1_TGC [symbol] | 262 | AAATCGCAATATACGAATACCACATAAACGGAAAGAATTCGTTTATTAGTTCCTTT |
| | | ATTTATTTACTATTACGAAAGTAA[G/C]CCATTTATGGTGTTATAATGACTTAATTA |
| | | TCTAAGATACTAATACATGACTATAAATTTAAAATCTGGCTACGGTTTAAT |
| RAD_kmer_0000037_1_TGC [symbol] | 446 | TGCAGTAATTTTAGGTAGGAAGTGCAAAATATGGGTCGGTAAAAAATCGCCGGG |
| | | TCAAATGGGTCATCAACAAAACCAA[C/T]AAATAACGATAAAATGCCAATATTCA |
| | | TTACATATTAATTAATAGCATTGACATGTTCATTAAAAATTAACCAATAACCTT |
| RAD_kmer_0000038_1_TGC [symbol] | 417 | AGAATAAGATAATTGGTTCTTAGAGTTTTTTTTAGGATGCAACCAATCTGAACTGTT |
| | | GGATGGCGCAACGAATCATAATC[A/T]TTGTTTGGAACCAAAATTTGAAGACAAG |
| | | TTAAAGGGCTAGATTTTTTTTAAACATCACTAATTTTAATGATAAATATACA |
| RAD_kmer_0000047_1_TGC [symbol] | 106 | TACGCATGTACCAGGTAAAGGGTTATGAGAGTTGGGCCAAGGCACCACTCCATGT |
| | | GGGCCTAAGGTACTCCAAAAATGA[G/A]GGCTTCACTTTAGCGTAACAGGACAT |
| | | GGGTATCAAGCATCCGTGATGAAACGCAAGGTGGGCACAAGCCCACCTATTGCA |
| RAD_kmer_0000085_1_TGC [symbol] | 210 | TCTTCTCTTCTATTCAATATATGTTCTCCGTCACTAAAATTTTTATACTGTTAAAAAT |
| | | GATTTTTTATTGCCCGGGATGGC[G/A]GGATCTCACTGGAAGCAACCTCTCTATCTTC |
| | | AAAGATGGATGTAAGGCTTGCCTCCATCCCACCCTCCCAGACCCTTACT |
| RAD_kmer_0000049_1_TGC [symbol] | 339 | TTGCCTCCATCCCACCCTCCCAGACCCTTACTTTCTGTGTGGGATATTACTGGGTAC |
| | | GCTTGTTTGTTTGTTTGTTTACT[T/C]CTATAATGTAGGTATGTTGGCAATAATATAG |
| | | AACCAACCCAATTATAAAAAGAAAAAAAAAAGATCATTCGTAGTGCTAG |
| RAD_kmer_0000052_1_TGC [symbol] | 352 | CTATGGTTATCCGTGAAAGGTATAATTCTGGAACACCCTATGGTTCGGGTTATAATT |
| | | TTTACTCGGTTTGTAATCGGATC[T/A]CTGACGGGAATTTAAATCAGATTTAAATTA |
| | | TGTACACAACGGTTCCGCTAACTCGATTAATATGTAATATTAATAAACGTA |

| #CHROM | Sample_1 | Sample_2 | Sample_3 | Sample_4 | ESTA Name |
|---|---|---|---|---|---|
| RAD_kmer_0000025_1_TGC [symbol] | G | A | A | A | GYSNP0001 |
| RAD_kmer_0000028_1_TGC [symbol] | C | G | G | — | GYSNP0002 |
| RAD_kmer_0000037_1_TGC [symbol] | C | C | T | T | GYSNP0003 |
| RAD_kmer_0000038_1_TGC [symbol] | W | A | T | — | GYSNP0004 |
| RAD_kmer_0000047_1_TGC [symbol] | G | G | A | G | GYSNP0005 |
| RAD_kmer_0000085_1_TGC [symbol] | G | G | A | A | GYSNP0006 |
| RAD_kmer_0000049_1_TGC [symbol] | C | T | T | T | GYSNP0007 |
| RAD_kmer_0000052_1_TGC [symbol] | W | T | A | A | GYSNP0008 |

[symbol]

indicates data missing or illegible when filed

The second set consisted of 192 SNP assays screened against the same set of 12 diploid accessions. Another 145 polymorphic SNP markers in this set were identified. A total 464 SNPs were found to be informative from both tests which were all used to screen single sample DNA (in contrast to bulked DNA in previous testing). A subsequent stepwise testing of these markers on single plant DNA sample was conducted to evaluate the effectiveness of each marker on individual plants. Four plants per accession were analyzed (total of 48 samples). Among the 464 SNPs, only 348 were found to be polymorphic when single sample DNA was used and these was the preferred set for routine analysis.

### Diversity Analysis and Parental Selection for Crossing

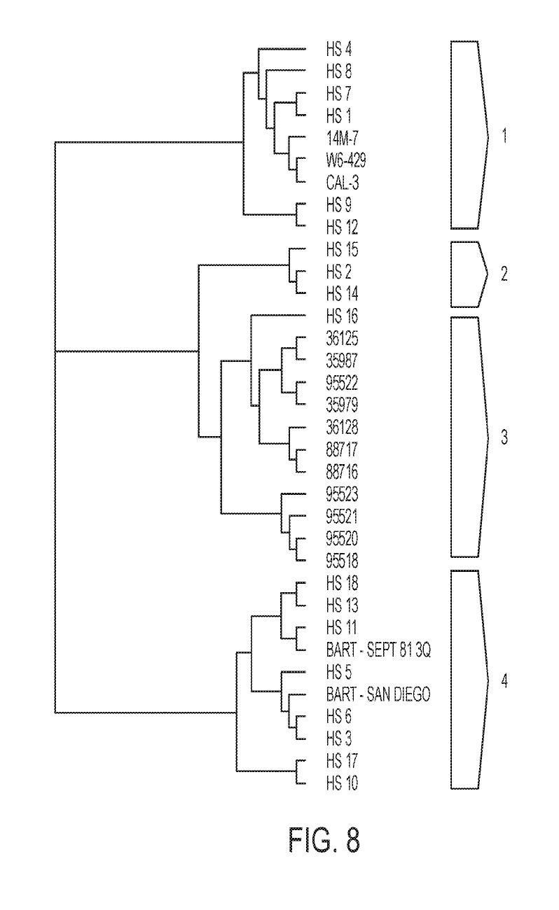

Leaf tissues from 1.144 diploid guayule germplasm were collected for SNP genotyping. The 348 SNPs set was used to determine the genetic similarity among these breeding materials and with the USDA diploid germplasm collection and representative polyploid germplasm. A summary neighbor joining tree from Nei's distances is shown in FIG. 8. There are four major clusters denoting germplasm groups. The USDA diploid germplasm W6-429 and Cal-3 grouped with 14M-7 and six HS. There is a distinct group containing G7-11 (8817, 8816) and other polyploids along with HS 16.

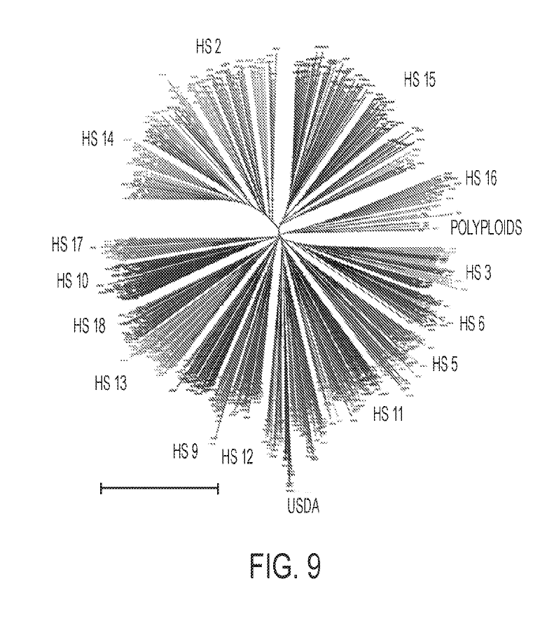

FIG. 9 shows a detailed tree with all individual plants color coded by HS and accession grouping. Some HS are genetically uniform with the individual plants having very similar genotypes (e.g. HS 10, 12, 13, 16, 17, and 18), while others show genetic admixture (e.g. HS 2, 5 and 6).

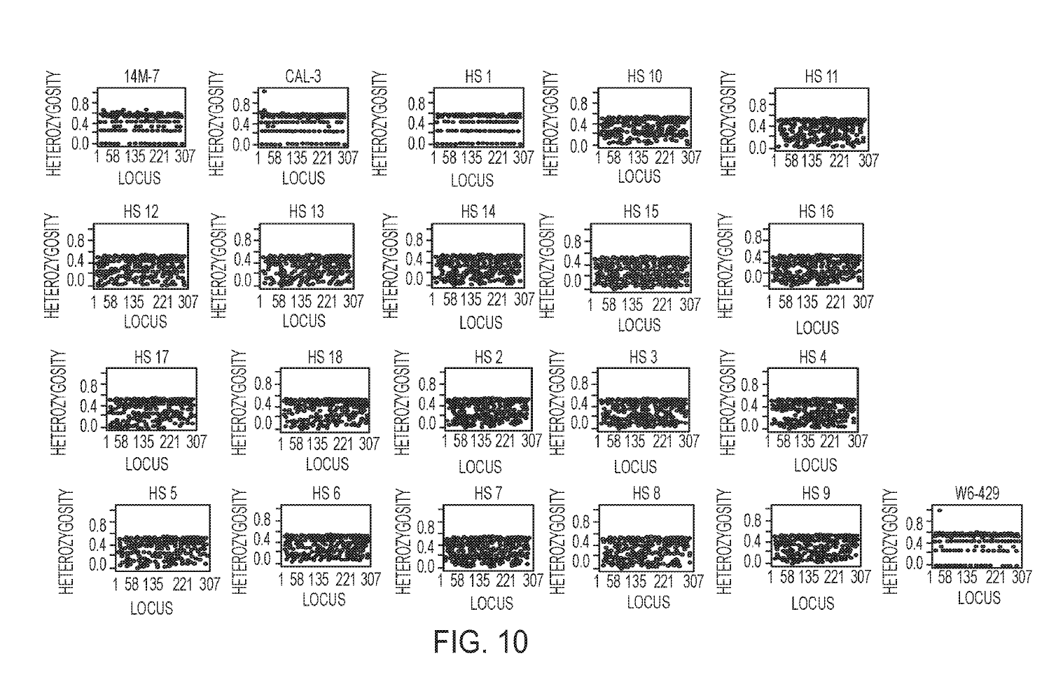

The mean total expected heterozygosity (gene diversity) of the diploid HS from the 348 SNP markers was estimated at 0.37. The highest heterozygosity was observed on 14M-7 (0.43) while the lowest on HS 14 (0.32). FIG. 10 shows the expected heterozygosity on all SNP loci of the diploids.

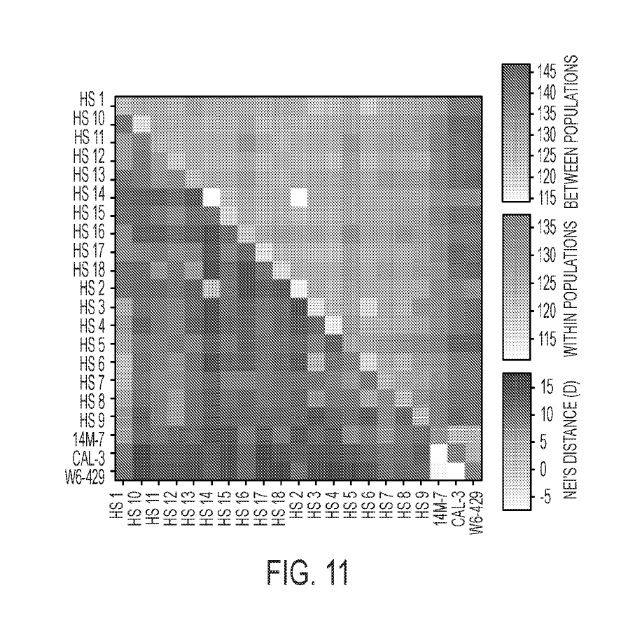

The similarity between and within the diploid accessions is shown in FIG. 11. Information from these genetic relationships are useful in selecting parents for the breeding program that are genetically distant (less similar).

C. Analysis of Rate of Apomixis in Guayule Germplasm

In another aspect of the invention, the rate of apomixis of guayule germplasm is determined and used in breeding guayule, including the selection of guayule germplasm to produce an advanced guayule plant, seed or plant tissue. In a further embodiment of the invention, a method for breeding a guayule plant comprises determining the rate of apomixis of one or more guayule plants, using the resulting information regarding the rate of apomixis of the plant(s) to identify one more select guayule plant(s) for use in guayule plant breeding to produce an advanced guayule plant, seed or plant tissue. In a further preferred embodiment, the rate of apomixis is determined by flow cytometry. In another preferred embodiment, the rate of apomixis is determined from leaf samples, seed samples, or both. The invention is also directed to guayule plants, seeds, and plant tissue generated from such methods of breeding. The plant tissue referenced in these methods includes the natural rubber produced by the guayule plant.

In another embodiment, a method for breeding guayule comprises (a) determining the rate of apomixis of one or more guayule plant(s). (b) determining the genetic pedigree of one or more guayule plant(s); and (c) using the information regarding rate of apomixis and genetic pedigrees to identify one or more select guayule plants for use in guayule plant breeding to produce an advanced guayule plant, seed or plant tissue. In a preferred embodiment, the DNA analysis is performed using DNA of single nucleotide polymorphisms (SNPs) or mono-, di-, tri-, tetra-, penta- or hexanucleotide simple sequence repeats (SSRs). In another preferred embodiment, the rate of apomixes is determined by flow cytometry. In another preferred embodiment, the rate of apomixes is determined from leaf samples, seed samples, or both. The invention is also directed to guayule plants, seeds, and plant tissue generated from such methods of breeding. The plant tissue referenced in these methods includes the natural rubber produced by the guayule plant.

The rate of apomixis in guayule germplasm was studied for enhancing guayule breeding. Guayule has a range of reproductive types of populations ranging from sexual diploid plants to facultative apomictic polyploid lines. Plant improvement has been a challenge in guayule through traditional breeding due to complications from facultative apomixis in polyploid collections.

As shown in Table 8, pollen viability also can relate to whether the plant is a polyhaploid, tetraploid or a diploid.

Guayule polyploids range from triploids (2n=3x=54) to octaploids (2n=8x=144). Polyhaploids result from reduction of a 4x (4x=72) maternal plant and no fertilization. On a ploidy analyzer, when sampling leaf tissue, they can appear the same as diploids but differ in reproduction.

Polyploidy was studied during the Emergency Rubber Project in the 1940's by counting chromosomes in root-tips or pollen mother cells, with appropriate stains. Use of new research technologies and equipment provide for advancement of the understanding of polyploidy in a way that was previously too labor-intensive to be practical. An example of this is the use of a desk-top ploidy analyzer (flow cytometry) that is a fast and easy technique used to measure embryo and endosperm ploidy. Flow cytometry (or ploidy analysis) allows hundreds of plants in a population to be analyzed and the ploidy of each to be identified. Since most lines or populations are facultative apomictic, this is very valuable information.

This technique was also expanded to not only analyzing the ploidy of a plant but to also determine the rate of occurrence of sexual and non-sexual types of reproduction. This method combined with molecular markers can be a nondestructive method for identifying new genetic diversity in a guayule breeding program.

In this experiment, a novel method has been employed using seeds produced by a single plant that utilizes the combination of embryo and endosperm ploidy number to detect the type of reproduction that occurs from this single plant, referred to as the rate of apomixis. Although this method is technically destructive, the knowledge of the rate of apomixis is valuable and once it is known, molecular markers can be identified in leaf tissue to distinguish between sexual and non-sexual (apomictic) types and allow selection of plants resulting from sexual reproduction to obtain greater genetic diversity for rubber improvement.

The USDA National Plant Germplasm System (NPGS) contains 51 accessions of guayule (P. argentatum) plus accessions that are interspecific hybrids. All except two diploid guayule collections reproduce by apomixis; the two diploid accessions reproduce sexually. The objective of this study was to estimate the rate of apomixis, as well as whether or not there was meiotic reduction in the megaspore mother cell, and whether or not there was fertilization to form an embryo. The germplasm that was the subject of this study included two basic types—2 accessions of diploids (sexual reproduction) and 48 accessions of polyploids (facultative apomictic). The polypolids range from triploids (3x=54) to octaploids (8x=144). All germplasm was publically available in the Germplasm Resources Information Network (GRIN) USDA collections.

Young leaf tissue of maternal plants and 100 progeny seeds from these plants were analyzed to estimate their rate of apomixis. The seed provided additional insight due to the ploidy levels in two different seed structures (embryo and endosperm). Diploid leaf samples were prepared in UV dye as a control to calibrate the ploidy analyzer. The seed samples were chopped in UV dye to prepare the sample with a 2x leaf sample and analyzed in the ploidy analyzer.

The study sought to identify which of the four potential reproductive classes the seed resulted from: (1) meiotic reduction of egg+fertilization; (2) meiotic reduction of egg+no fertilization; (3) no meiotic reduction of egg+fertilization; and (4) no meiotic reduction of egg+no fertilization. The results potentially indicate that the earlier assumptions of: (1) embryo sac development and (2) normal meiosis in the male (pollen) are not always the case. Results from this study indicated that off-type progeny (seed) would not have been identified if only leaf samples were used, resulting in an over-estimate of the rate of apomixis for the accession. Segregation in triploids is an example of this.

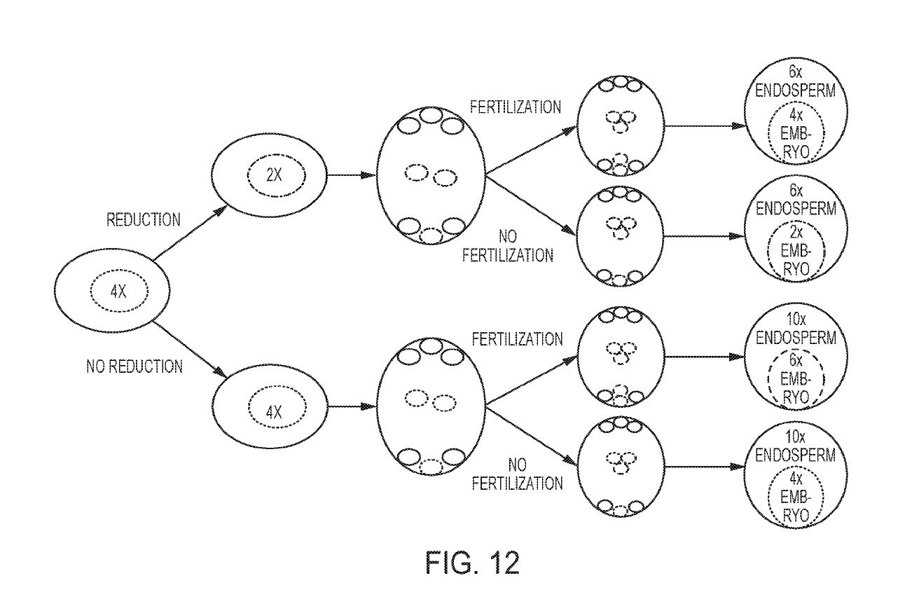

For example, a tetraploid×tetraploid could involve the reproductive classes as depicted in FIG. 12.

These results can help guide a guayule breeding program and better utilize plants resulting from reduction and/or fertilization causing new recombinations. The technique will also help to better characterize new breeding lines and the expected amount of variation from generation to generation.

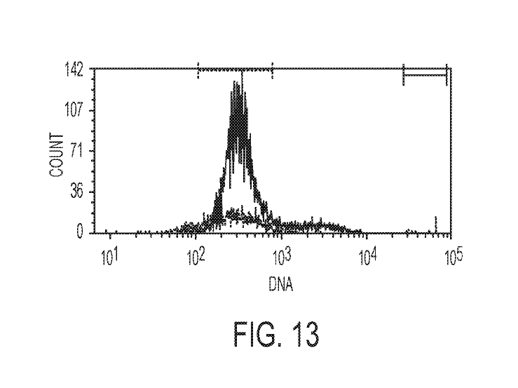

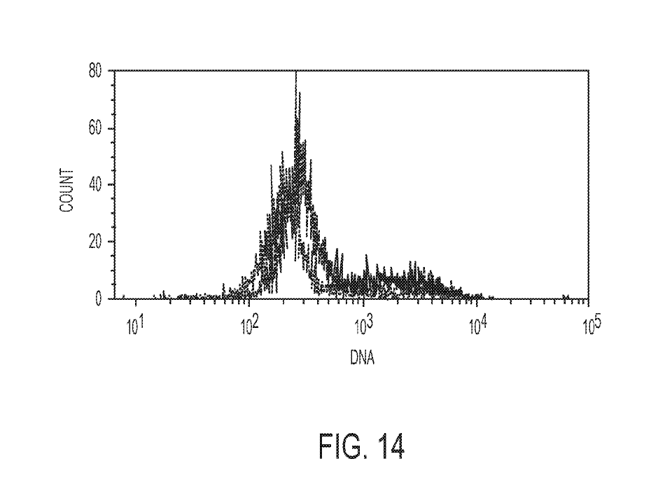

Seed maturity and nuclei staining was also studied. Seeds from plant heads at different stages of development (early, mid and late stage) were collected. As shown in FIGS. 13-14, results indicated that embryo ploidy was detectable at all development stages.

The USDA collection R1100-4x resulted in a 93% rate of apomixis. The USDA collection 11604-2x polyhaploid resulted in a 0% rate of apomixis. Further studies and analysis will further expand the usefulness of this technology in developing improved guayule plants and the rubber produced by such plants.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

TABLE 9
SNP Molecular Markers from Guayule

| | | | Sample | Sample | Sample | Sample | |
|---|---|---|---|---|---|---|---|
| #CHROM | POS | SEQ | _1_2 | _3_2 | _2_2 | _4_2 | EST Name |
| RAD_kmer_0000025_1_TGCAGGTGGCGCGGCTGGACCATAG | 107 | CGCCACACATAAATAATCAAACCTTATCCGTCACCACTTGAG | G | A | A | A | GYSNP0001 |
| TCCAATGAGCGGCGGCGGCACGGCGGCAAAGAGTTTGGT | | AAAAACCCTATACAGGGCGGCATGCCGCCACTGTCGCT[A/G] | | | | | |
| TTGTTCTTGGATGATGACGCAAGCAAC_NODE_1_length_547_ | | CTGCCCTGTCCGGCCGCCATTTGTTTTTGTTTTCTGTTTCGTCT | | | | | |
| cov_74.769653 | | TTCTTGTTTCCTGTTCTAAAAAGACTAGGTCTAGGT | | | | | |
| RAD_kmer_0000028_1_TGCAGGTGCACCTATTCCCGCACCA | 262 | AAATCGCAATATACGAATACCACATAAACGGAAAGAATTCGT | C | G | G | — | GYSNP0002 |
| GGTAAATTTTATTTGCCGTACAAAGTTTTACTATTGTACAT | | TTATTAGTTCCTTTATTTATTTACTATTACGAAAGTAA[G]CC | | | | | |
| TAAATAATTTATAAATCTGAAATAA_NODE_1_length_500_cov_ | | ATTTATGGTGTTATAATGACTTAATTATCTAAGATACTAATAC | | | | | |
| 93.066002 | | ATGACTATAAATTTAAAATCTGGCTACGGTTTAAT | | | | | |
| RAD_kmer_0000037_1_TGCAGGCTGCAGCATGTGTCATTGT | 446 | TGCAGTAATTTTAGGTAGGAAGTGCAAAATATGGGTCGGTAA | C | T | C | T | GYSNP0003 |
| TTAACTATTATATTACTTTAAAGTTTTTACTTTGCTTATGTT | | AAAATCAGCCGGGTCAAATGGGTCATCAACAAAACCAA[C/T] | | | | | |
| AAGCTTTTGTTTACTTAAGGTTAT_NODE_1_length_583_cov_ | | AAATAACGATAAAATGCCAATATTCATTACATATTAATTAAT | | | | | |
| 88.401375 | | AGCATTGACATGTTCATTAAAAATTAACCAATAACCTT | | | | | |
| RAD_kmer_0000038_1_TGCAGGTAATTAAAAGAAAAAGTT | 417 | AGAATAAGATAATTGGTTCTTAGAGTTTTTTTTAGGATGCAAC | W | T | A | — | GYSNP0004 |
| AGACAACAGTTAGTTTCATCACATTTAAGTTCCTTTTTCAC | | CAATCTGAACTGTTGGATGGCGCAACGAATCATAATC[A/T]TT | | | | | |
| CCAAACAATTCACAGCAACAAAATTT_NODE_1_length_600_ | | GTTTGGAACCAAAATTTGAAGACAAGTTAAAGGGCTAGATTT | | | | | |
| cov_63.478333 | | TTTTTAAACATCACTAATTTTAATGATAAATATACA | | | | | |
| RAD_kmer_0000047_1_TGCAGGATTGATTGTGGCTATGGTC | 106 | TACGCATGTACCAGGTAAAGGGTTATGAGAGTTGGGCCAAGG | G | A | G | G | GYSNP0005 |
| ATGGCGGAGAGCGGAGACAAGTGCAATAGGTGGGCTTGT | | CACCACTCCATGTGGGCCTAAGGTACTCCAAAAATGAT[G/A] | | | | | |
| GCCCACCTTGCGTTTCATCACGGATGC_NODE_1_length_206_ | | GGCTTCACTTTAGCGTAACAGGACATGGGTATCAAGCATCCG | | | | | |
| cov_132.150482 | | TGATGAAACGCAAGGTGGGCACAAGCCCACCTATTGCA | | | | | |
| RAD_kmer_0000049_1_TGCAGGTACTCTTTTTAAAGGATGC | 210 | TCTTCTCTTCTATTCAATATATGTTCTCCGTCACTAAAATTTTT | G | A | G | A | GYSNP0006 |
| AGTAAGAACTAGCACTACGAATGATCTTTTTTTTTTCTTTT | | ATACTGTTAAAAATGATTTTTATTGCCCGGGATGGC[G/A]GGA | | | | | |
| TATAATTGGGTTGGTTCTATATTAT_NODE_1_length_426_cov_ | | TCTCACTGGAAGCAACCTCTCTATCTTCAAAGATGGATGTAA | | | | | |
| 113.995308 | | GGCTTGCCTCCATCCCACCCTCCCAGACCCTTACT | | | | | |
| RAD_kmer_0000049_1_TGCAGGTACTCTTTTTAAAGGATGC | 339 | TTGCCTCCATCCCACCCTCCCAGACCCTTACTTTCTGTGTGGG | C | T | T | T | GYSNP0007 |
| AGTAAGAACTAGCACTACGAATGATCTTTTTTTTTTCTTTT | | ATATTACTGGGTACGCTTGTTTGTTTGTTTGTTTACT[T/C]CTA | | | | | |
| TATAATTGGGTTGGTTCTATATTAT_NODE_1_length_426_cov_ | | TAATGTAGGTATGTTGGCAATAATATAGAACCAACCCAATTA | | | | | |
| 13.995308 | | TAAAAAGAAAAAAAAAAGATCATTCGTAGTGCTAG | | | | | |
| RAD_kmer_0000052_1_TGCAGGTATCTTGGTAACCATGGGA | 352 | CTATGGTTATCCGTGAAAGGTATAATTCTGGAACACCCTATG | W | A | T | A | GYSNP0008 |
| ACACCGCCACCACCTACTCGGTAAGGGTTGGCGGGTTATG | | GTTGGGGTTATAATTTTTACTCGGTTTGTAATCGGATC[T/A]CT | | | | | |
| GCCTCAGGTAGTTTTACCGACGAAGA_NODE_1_length_500_ | | GACGGGAATTTAAATCAGATTTAAATTATGTACACAACGGTT | | | | | |
| cov_64.052002 | | CCGCTACTCGATTAATATGTAATATTAATAAACGTA | | | | | |
| RAD_kmer_0000053_1_TGCAGGATAATGAAGAGCGGAAGC | 328 | CAAAAACCGTTATCTGCAAGGGAGGATGCATCAAAGATCAG | A | — | W | A | GYSNP0009 |
| AACACGACATATGGAAGGAATTGCAGATGGATCTCCTGA | | CATTCGAGCATGTACATGTGTTGCCAACATTGCAAACAG[T/A] | | | | | |
| ACCAGCTCTAGCTGTCAGCAGGTCAGCA_NODE_1_length_643_ | | ACAAAAGATCTCATTGATAAAGAATTATCAGCGTTTTCGTGT | | | | | |
| cov_141.555206 | | TGTCCTGATAATAGGCTTTACATGCTTCCATCCACTCC | | | | | |
| RAD_kmer_0000057_1_TGCAGGTTTCACTTTGGGATGTTGA | 86 | TATCAATTATGTCCAAAATAATGACTCTTGTATAGATTGAGTT | G | T | G | G | GYSNP0010 |
| ATCTAGCACTTGTCCCTCATTCACATCAACAAGTAGTGAG | | TGCTTCTACACTTTTAATACATTTAATGCTGATTTCA[G/T]ATT | | | | | |
| GTCACGTTGTGGAAGCTGTGGCTAGA_NODE_1_length_548_ | | TTCAACTTGATGTAAAAGGCTTTCAAGATGTCCATGCTTCTTT | | | | | |
| cov_79.206207 | | AAACAAATATCTTGCACAAGAAGAGTTCATTATT | | | | | |
| RAD_kmer_0000059_1_TGCAGGACCGAGGCCGACCTCGATA | 546 | AATATATATTTATGAAAAATGGACAAAAGTGTTTGTAGTTGA | T | C | C | C | GYSNP0011 |
| TCTGTAAACAGTTCTGCATCCAAAAACTCAGCCATGTTCT | | CTGAATTAGACTTCTACCAAATGATAAAAATCTTGCAG[C/T]T | | | | | |
| CTCTGAAGCTTTTCATGTAGTCACTG_NODE_1_length_615_ | | ATACAGTGACTACATGAAAAGCTTCAGAGAGAACATGGCTG | | | | | |
| cov_121.310570 | | AGTTTTTGGATGCAGAACTGTTTACAGATATCGAGGTC | | | | | |
| RAD_kmer_0000062_1_TGCAGGATACACAACTTTTACTTTG | 213 | GGACGTCTTTTGTGCTGGGAGCCGGTTGGAGGGTACCTACAG | W | W | W | W | GYSNP0012 |
| GGATCTTGCGATGGATGAACTTGTAGTGCCACTTCGCCGT | | GGACCACATTGTCCCAATGAGATGACGGGGTCCTACTG[A/T]A | | | | | |
| CCACCTGGTGGGTCCCCCACGTTCAG_NODE_1_length_274_ | | CGTGGGGGACCCACCAGGTGGACGGCGAAGTGGCACTACAA | | | | | |
| cov_103.233574 | | GTTCATCCATCGCAAGATCCCAAAGTAAAAGTTGTGTA | | | | | |
| RAD_kmer_0000063_1_TGCAGGTTCATAAACAAAGGTAAAT | 193 | TTGGTTCATTTTTTGTAAAGGTAAGGAAAATATTACTTCCAAC | G | K | G | K | GYSNP0013 |
| CTGCGTACATGTGTCTAGTCTAATAAAAATAATGGAATGC | | CACCATTCATCAGTTAAATGCAACTTTTTTTTTTTTT[G/T]CTTT | | | | | |
| CAATATTATAAACATTAGTAGAATTA_NODE_1_length_451_ | | ATTTGAAATCTGATAGTTTTCCATGCTCAACTTTGCAGTCAAA | | | | | |
| cov_91.121948 | | ATTGAGTCCTGATGCAGGCCATATTCTTGGTGG | | | | | |
| RAD_kmer_0000064_1_TGCAGGGTTTGGCGTTCCAATTAAT | 363 | CATATCTAATTTATTTCAATAAACCAATTGTGATGGTTGAAAA | K | T | K | T | GYSNP0014 |
| AGATGATGTTCTAGATTTTACTGGCACATCATCATCTCTTG | | CATCTGAATATATTGATCTCTTCATTTGTATCTTCAG[T/G]TTT | | | | | |
| GAAAGGGCTCATTATCTGACATTCG_NODE_1_length_580_cov_ | | TTAGTGATCAGATTCAAAATCTCTATACATTTATACTGACCCG | | | | | |
| 124.134433 | | TAATCAAAAAGGTCTCATAAAGGTTATAGCTAGT | | | | | |
| RAD_kmer_0000068_1_TGCAGGTACTCAACCTGATGATGTT | 232 | ATGGACGCAGGGTTATTGAGCGAAGCAAGTTCTTCTAGAAGC | T | G | G | T | GYSNP0015 |
| CTTGGTTTGGCAAAGAAAGGCGTTCCAAAATTGAATAGC | | TCGCGTTCTTGGTCACTAACACAACCAATATCAGCACA[G/T]T | | | | | |
| CATCCATTCGTGGCGATCATTTGTTC_NODE_1_length_520_ | | TAAAATACAGAGTGATTAGGAACAACTCATTGTACACATTTC | | | | | |
| cov_83.801926 | | CATCTAAACTTTACAAAATAATAATAATAATAATAAT | | | | | |
| RAD_kmer_0000071_1_TGCAGGATACGATGCATAAAACTGA | 265 | TGTTCAATGCGTATAGACATAGTGTAGTAATTGAATTATACA | T | A | A | A | GYSNP0016 |
| TCACCTCTAGAATAACCAGCTCTGGTCCTGCAACiAATTAT | | GTTCTCTTCTTAGGTTAATTGCATGAGAAACCGATTCT[A/T]GT | | | | | |
| ATAAAGAGCAAAAGAGAAATGATATT_NODE_1_length_610_ | | TATGCACACTAGGTGTTAGATGAAATGCCTCACTGGGATTTG | | | | | |
| cov_64.655739 | | TATGATTTTTTTACAGGAAATCTAAAATCGTCCTCA | | | | | |
| RAD_kmer_0000071_1_TGCAGGATACGATGCATAAAACTGA | 460 | ATGATGGGTGGAATGAAATTTTAAAGATTCAGAAGTTTCGTA | A | C | M | — | GYSNP0017 |
| TCACCTCTAGAATAACCAGCTCTGGTCCTGCAAGAATTAT | | GAGCAGTGGGAAAAACAATATTCTTTTCGTTCTCGGCT[C/A]T | | | | | |
| ATAAAGAGCAAAAGAGAAATGATATT_NODE_1_length_610_ | | CATTAGCTATGCATACAACAACAACACGTACGTATACTGTTA | | | | | |
| cov_64.655739 | | TGAGTCTGCTTTCTAAGTAAATTTGTTATTCCTTGAA | | | | | |
| RAD_kmer_0000077_1_TGCAGGTCCTATCTACGTTTCTTAGC | 243 | CGGACATTATTTCTGTATTTAGACTTTTATCGATCGACGTCTA | T | A | W | T | GYSNP0018 |
| TGTATCTCTAATTTATGTACCTGCAAATGTTTTTTTTTGGA | | TGTTTTTTAACTTTTCCGGCTCATCTTCCTCCGAAGC[T/A]TTC | | | | | |
| ATCCGTGATTTTCGATCATAGTCC_NODE_1_length_533_cov_ | | ATCCGAGTAGTCACTACGCCCTACCCTATCGCTGAGTCTTTGG | | | | | |
| 161.367737 | | AAGCGGTTTCACTCCTTTATAGTTACCATAATTC | | | | | |
| RAD_kmer_0000079_1_TGCAGGCCTGGTTTGTTTTGATTCGC | 95 | TGGATTTGTAATTGGAAACTCATTATAATCTTAGGGGCATAG | A | C | A | A | GYSNP0019 |
| ACTAGAAAATGACATGTTTTGACCCGTTACCCAATACCTG | | AGAAATAAACACATACTCATGATTCATAGAAGAACACA[A/C] | | | | | |
| AAGTCAACATCTTGCAAATTCTAAT_NODE_2_length_387_cov_ | | GGGATTTGCAACAAATCCGAAAAACATAAACCTGAACAGCA | | | | | |
| 52.521965 | | CGGTCTCTGTCGGTTTCATACACAGTCACACGTAAACCT | | | | | |
| RAD_kmer_0000083_1_TGCAGGTAGCTTAACGAGCCGTAGC | 91 | GGAATCTGTGTCAAAGGAGGGAATCTCCAGACTTTGATTCTC | Y | C | Y | C | GYSNP0020 |
| TTTCAGGAATACTTCCAGTTAACGAATTGTAAGACAGATC | | AACAACAACCTTATCACAGGAACAATCCCCAAGTCGAT[T/C] | | | | | |
| AAGGTAGATCATGCTGCCATTAGCTT_NODE_1_length_577_ | | GCCAGCTGCACGAATCTGATGTGGGTTTCGCTTTCGAACAAC | | | | | |
| cov_97.315422 | | AAACTCGAAGGCGAGATCCCTGAAGGCATTGGGAATCT | | | | | |
| RAD_kmer_0000089_1_TGCAGGAATGTTTATAAAACAAGGA | 273 | GTTACCCAACCTGATCGACCTGCTTTACTAAAAGGAATGTGG | C | C | T | C | GYSNP0021 |
| CTTGGTTCATTTGTATGTTCCTCTTTTTCCCTATATTATACC | | AAAACTCTCACCTGAAACACACTAGAACTCGAAGAAGA[T/C] | | | | | |
| CATGAATATAAACCATTTACATAT_NODE_1_length_624_cov_ | | TCTCCATGTTTTGTATATCCAAACCCGCAGCCTTTGCATAATC | | | | | |
| 116.897438 | | TTACAAGTATATTTGTTACAGGAATCCATGATCGGTT | | | | | |
| RAD_kmer_0000089_1_TGCAGGAATGTTTATAAAACAAGGA | 423 | CATGATCGGTTATCAAATGCTGATAAAAGTGATGTTGGCAGG | A | A | G | A | GYSNP0022 |
| CTTGGTTCATTTGTATGTTCCTCTTTTTCCCTATATTATACC | | CTAAGAGTTGCTGCTTGGTTGCTCTCAAAAGCAGCCAA[G/A]A | | | | | |
| CATGAATATAAACCATTTACATAT_NODE_1_length_624_cov_ | | ACTCCTTGTACTGAACTAATACAGAAATTGATTGCAGAAGTA | | | | | |
| 116.897438 | | GATCTCTTAGTTCTGCACTTGATATTCTCGGGTCGCT | | | | | |
| RAD_kmer_0000106_1_TGCAGGAAGTAGGTGTGAGCAGAA | 157 | ATCACCAATTTCATGTCAGTCTTCATTGCCATGATATTATTTC | C | C | T | C | GYSNP0023 |
| CATGGCCGGAACCATGGCAACCGAACCGAACAGTGAGTA | | GTTTGAGTTTGGTCCCAATACGATCATTAGGTGGGCC[T/C]CC | | | | | |
| CCACATAGACCGACCCATTTGATAATTA_NODE_1_length_618_ | | TTATAAATATTTGTCCATATTATATTACTTTGAAAATAATAAT | | | | | |
| cov_145.288025 | | TTTTTTACATAGCATTTAGCTTTAAAAAAATACGA | | | | | |
| RAD_kmer_0000106_1_TGCAGGAAGTAGGTGTGAGCAGAA | 404 | CAAACATAAGTACACACACATACACATATATACATCAGAACA | C | S | C | G | GYSNP0024 |
| CATGGCCGGAACCATGGCAACCGAACCGAACAGTGAGTA | | CACATAAAAACACACATATCAATATACACACACAAGCA[C/G] | | | | | |
| CCACATAGACCGACCCATTTGATAATTA_NODE_1_length_618_ | | ATGGATACATACCTACAACTCAGAATGCACAGATATGCATAT | | | | | |
| cov_145.288025 | | CAGCATATTTCATATTCGGGTCTAGGGGTTCTAACCTA | | | | | |
| RAD_kmer_0000108_1_TGCAGGTCATATCCACTGAACATCA | 330 | ATTGCAGATAGTGAGGTTTATTTATTTGTTTGTTGTTGCATTTT | G | G | T | G | GYSNP0025 |
| AAACAATCTAAGAAATCAAAAACTACAATCTTCATAAAA | | ATTTATTTTATTTGGTTTATGAATGAAAATTCGTCT[G/T]TTAA | | | | | |
| AAGAACAACATTATCTCCATTGATGTT_NODE_2_length_471_ | | GGGTTTATGGAAATTCGTAAATGACCAACAGTATTGTATTTA | | | | | |
| cov_47.454353 | | TTTTATCATAAAGAATTTAATTTTTTTTATTTT | | | | | |
| RAD_kmer_0000117_1_TGCAGGAAAGAGAAGGCCAAACGA | 106 | GGACCAAGTCTGCACCAAGAATAGCTCGCACAGACGTTTTTT | — | A | R | A | GYSNP0026 |
| GGTTGTGATTGGCGAGCCAGTCGTAGAGAATAGGGACCA | | AATGAGACGTATTAGAGAAAACAAACAGTCTACAGATG[A/G] | | | | | |
| GAGACTTCCACTGTGTGTAACGTTCGTT_NODE_1_length_435_ | | CAATGTATGCGCGTGGTCTGCGCGGCGCAGACATAAGAGCCT | | | | | |
| cov_46.694252 | | GAGCAAACATTTTTTAAAAAAACAAACACCACCGTAAT | | | | | |
| RAD_kmer_0000121_1_TGCAGGGTCCTCAAATAATTCAGCA | 125 | GGAGACAAATAAATCCCGGCTCCATAAACAAGTTGTTTTAAC | G | R | R | R | GYSNP0027 |
| GGATCCACAACGTTGCCAAAATTGTGGAGCTTATGCAAAT | | GATTCTTGACTTGGAGAAACATTGCCCGGTAACAAATC[G/A]G | | | | | |
| CTCTATTGCAACATCTTACTTGGATC_NODE_1_length_538_ | | CTGATGCAATCGATCCTTCAGAAAGATCATAAACCGAAACCA | | | | | |
| cov_211.905197 | | TTTTCCCATATGTTACAACACCGATTCTTGTTGTGGG | | | | | |
| RAD_kmer_0000121_1_TGCAGGGTCCTCAAATAATTCAGCA | 181 | AGAAACATTGCCCGGTAACAAATCGGCTGATGCAATCGATCC | C | T | T | Y | GYSNP0028 |
| GGATCCACAACGTTGCCAAAATTGTGGAGCTTATGCAAAT | | TTCAGAAAGATCATAAACCGAAACCATTTTCCCATATG[T/C]T | | | | | |
| CTCTATTGCAACATCTTACTTGGATC_NODE_1_length_538_ | | ACAACACCGATTCTTGTTGTGGGTGGAAGTGAATCCACAAAT | | | | | |
| cov_211.905197 | | GCATGTAAAGAACTCTGCAGATGTTGAAGATGCGGTT | | | | | |
| RAD_kmer_0000129_1_TGCAGGTACGGTTGTCGATTCCAAG | 372 | AAAAATTAGTTCCATAAAGGGTGAAATTCAAATTTTGACGAT | T | T | C | T | GYSNP0029 |
| ATATGTCATCCCACAGAGTTTGATTTCTACCTGTGCAGCC | | AAAGACAGACAGACATACGTGTAGCACAGGTTATTTGT[C/T] | | | | | |
| ATGCTGGCATTCAAGTATGTATTCAT_NODE_3_length_460_ | | AGTGACTGCAGAGCATCCGCTTGGAACTTGTTCTCATCCCAG | | | | | |
| cov_38.752174 | | AGCACATGGTAATGGGCTGGGCGGCTAGTACCCTGTTC | | | | | |
| RAD_kmer_0000138_1_TGCAGGTGGCTTTTTACTTTGACCCT | 320 | CAGGTCGGCAGGTTAATTGGGTTGTAAATGGGTTGGTCAGGT | T | C | T | T | GYSNP0030 |
| TTGATTTTAGTTCTAAATAATAGTAATTTCTTGATTATCTT | | AAAAGGGTTGCACTTATTATTTATATTTTGTTCTACAT[T/C]TT | | | | | |
| GATTCCAACAAAAACCTTCTAAAT_NODE_1_length_632_cov_ | | AAACGAGAGGGAGTTCAAATCCTTTATCCACAACCCAACCTA | | | | | |
| 100.169304 | | CGACCTGTCTATCATACCAGGCTTGGCGGTGTCATG | | | | | |
| RAD_kmer_0000140_1_TGCAGGAAGTAGGGGCGAGCAGAA | 123 | CCTATGAAAACATCAACAATTTTAAGTTTTTAACTTTAAATAT | C | T | C | T | GYSNP0031 |
| CAAGGTCTGAACCGTAAGAACCATGGAACCGAAAAGTGA | | GTAGTTACATAGAAACAGTTAGCCCTATATTCTACCA[C/T]AT | | | | | |
| GTACCACAGTGACCAACCCGTTTGGTAA_NODE_1_length_461_ | | ATGCAAATGAATATGTAATTTGAAAATTACCATAGACATTTA | | | | | |
| cov_58.613884 | | TCAGATGGGGCAGACCCTGAGTAAAAGGTTGAAAGA | | | | | |
| RAD_kmer_0000141_1_TGCAGGATGGAAAATAGAAGCATT | 309 | CTCATGACACGGTAGAATTGGCCAGATGTATTGAAAAAACCA | A | — | A | W | GYSNP0032 |
| CCAAAGAGCTCCTCTAGCAACCATCACAGACGAGGCGCC | | TGGTGTCTGCGGTAGCCGTCCATGGAAGGTAATAATGT[A/T]A | | | | | |
| TACAAAAAAATGTTATTGCACATTCAAA_NODE_1_length_638_ | | TATATAATATTATTCATGTGATAAACTTTTCTTTTTAATTTTTAC | | | | | |
| cov_81.354233 | | AGTTTGGTAATATATTTTTATATATTTTGTTAAGT | | | | | |
| RAD_kmer_0000141_1_TGCAGGATGGAAAATAGAAGCATT | 407 | TGTGATAAACTTTTCTTTTAATTTTTACAGTTTGGTAATATATT | C | — | T | C | GYSNP0033 |
| CCAAAGAGCTCCTCTAGCAACCATCACAGACGAGGCGCC | | TTTATATATTTTGTTAAGTGAATCGCAGAAGAGTTG[T/C]GGA | | | | | |
| TACAAAAAAATGTTATTGCACATTCAAA_NODE_1_length_638_ | | TAGACCAAGAGATCCTGCTAATTGGAGTGAAATCGCAAGTGT | | | | | |
| cov_81.354233 | | TGTAGATGCTCTTGCTATACCCGTCATTGCCAATG | | | | | |
| RAD_kmer_0000146_1_TGCAGGTACCTTCCGACGACATGTC | 407 | CTGTATATGATGGCAGGAGCCATAGTCATCACTCATCACCTA | — | G | A | A | GYSNP0034 |
| GTCTTCAATCGGACAAAACCATGACGTAAGTCAAAAGTTT | | CCATACTTCATAATTTTATTTTTAATTTATTTAATCAT[A/G]AA | | | | | |
| CTGGCCAGGATCTGCAACCCCATTTC_NODE_2_length_543_ | | GTAGTAATATTATAAGACCACACATATCTTCATCCCACAGTT | | | | | |
| cov_35.797421 | | GCTGTCACAGTCAACAAAGCACACCACCTCCACCAC | | | | | |
| RAD_kmer_0000155_1_TGCAGGTCATTGCATCATGGGAGAA | 116 | TTAATTATCTGGTATTTTATTTATCAAGTTCTATTAAATTATTT | T | T | G | T | GYSNP0035 |
| GAGATTGCTGGCTTAAAACACATGTTTAAGATGATAGATA | | GTAAAAACCTACAACTATACGAGTTTTAGTGAGTTT[G/T]CTA | | | | | |
| TAGGCAACATTGGTTACATTACTTTT_NODE_1_length_581_ | | TTTATTTATTATTTTTCTCTGTCTTTTAAAGAATTTATGTTGTT | | | | | |
| cov_80.139412 | | ATTTATTATTTATTTTATTTGTTTTAAAAAAAT | | | | | |
| RAD_kmer_0000163_1_TGCAGGCAATATGACCTCAGAAGTC | 146 | CTTCATGCAAATTGTGCCATTTTGTTGGCTTGTTATGTATGTG | G | R | R | R | GYSNP0036 |
| TTAACAGTATTTACATGTAAAATATTAATGTTTGCATTATG | | TAATTTGTTTCTCGAAAACTAAGTTTGGTATAAATGT[G/A]TG | | | | | |
| TCCACAAACTCAATTTTCAACAGGT_NODE_2_length_360_cov_ | | ATATATTCTTTTTACTATATTCTCCTAAAACGCATGCTTACTCT | | | | | |
| 53.000000 | | TTGTAGCATTTGCATTATTTTACCATGTATTTCC | | | | | |
| RAD_kmer_0000169_1_TGCAGGCTGCAGGTTCTAGTGTATT | 296 | ACTTGCATACTTTCTTAAAATGGTTTGTAATTTTTGTCATCATT | T | T | C | T | GYSNP0037 |
| TATCCTAGAAAATCTAGGAATTGCTGTTCAAAAAGAAAA | | ATGTTTAAAATAGTTAAAACTGAGAAAAAAAAGTAC[C/T]AT | | | | | |
| AAAAAATCAGGGTATGTACTACTAGTA_NODE_1_length_402_ | | TAGCAGGCATACCCGAGCTTTCATCATTATTAGATTTTTTACT | | | | | |
| cov_60.300995 | | AGTAGTACATACCCTGATTTTTTTTTTCTTTTTGA | | | | | |
| RAD_kmer_0000171_1_TGCAGGAATGAGTCTATATAGGGAA | 359 | GATTTTGTTAGAATGTATTGATATTTTGACATTTCCATATAAA | G | G | K | T | GYSNP0038 |
| GAATGGAAGATTGTAAAGTATCAGTACTCTTGCTTTACTA | | TCTTATAGGAACCAGATGCAGCTGTTTCAGTACTTGG[T/G]GA | | | | | |
| AAATTGAGTGATTAAAAACAATATAT_NODE_1_length_528_ | | AATGGATGCTTGGATTATTTTGCCCTTTTTCTTGCAAGGAGGC | | | | | |
| cov_103.505684 | | CGTTTCACCATTGATGACGTGCATTATGTAGCTGA | | | | | |
| RAD_kmer_0000171_1_TGCAGGAATGAGTCTATATAGGGAA | 451 | TTGGATTATTTTGCCCTTTTTCTTGCAAGGAGGCCGTTTCACC | G | R | R | A | GYSNP0039 |
| GAATGGAAGATTGTAAAGTATCAGTACTCTTGCTTTACTA | | ATTGATGACGTGCATTATGTAGCTGATTCTGATAGGT[A/G]CT | | | | | |
| AAATTGAGTGATTAAAAACAATATAT_NODE_1_length_528_ | | ATAAACTGAAATATATTGTTTTTAATCACTCAATTTTAGTAAA | | | | | |
| cov_103.505684 | | GCAAGAGTACTGATACTTTACAATCTTCCATTCTT | | | | | |
| RAD_kmer_0000172_1_TGCAGGTTAAAGACTCAATGAGCAT | 109 | AATTTCATCAGACAAAACATAAACGCAGCCCAAACTAGCACA | Y | Y | T | C | GYSNP0040 |
| TTGGCATTTGCTCTAGTATTGTTTAGCTGGGATGATGTCA | | AGTTCCTAGCTACATATTATTATACATTACTACAGTTT[T/C]CA | | | | | |
| TATTCTTGAATGTTTACCTATTTTA_NODE_1_length_604_cov_ | | ACCTATTCCCACGGTCCCTTAAAAGTTGAAAGAAAAGGGGGA | | | | | |
| 170.134109 | | ACGTTAACCTGCTTGTGCCAAAATGGGTCGAGTAGG | | | | | |
| RAD_kmer_0000172_1_TGCAGGTTAAAGACTCAATGAGCAT | 344 | TGACCCAACTCACCTGGAGAGAAGTCTCGAGTGGTGAAGAG | A | M | M | A | GYSNP0041 |
| TTGGCATTTGCTTCTAGTATTGTTTAGCTGGGATGATGTCA | | GCAGCGACCTTTGTCTGGAAGTGTGGAAACTGATAAACC[C/A] | | | | | |
| TATTCTTGAATGTTTACCTATTTTA_NODE_1_length_604_cov_ | | CGGTCATGTTGCAGTAGCCTTTGTAAATCTTCCATCGTCAAA | | | | | |
| 170.134109 | | ACCAGAAAGCTCTAGTCTGCCTAGCTGGCGATACAATT | | | | | |
| RAD_kmer_0000174_1_TGCAGGTCGACTAAAGCTGCTCAAG | 231 | CTTATGCGAGTATCCCAAACATATAATTTTCGAGTTCGTCAGG | T | — | C | T | GYSNP0042 |
| AGCCCAATCACAGAGAACCATTAAAAACTATAAGGTATT | | CAGTTTCTCAAGCCTCCGACAGCCTTCAAGCTCCAAC[C/T]TT | | | | | |
| CCGTCTCTCAGCTTACTTTTAGTTGCC_NODE_1_length_658_ | | TGAGTTCAAATGTAGTAACAGTCACTGCATCATCCACCAGC | | | | | |
| cov_66.031914 | | AGATCAGTCCATAGATCAGATCCCCATCATCCACCA | | | | | |
| RAD_kmer_0000174_1_TGCAGGTCGACTAAAGCTGCTCAAG | 496 | TGTGCTAAATTTCATTATTTGTTAACTTTCAAAGTCAAAACTT | Y | — | T | C | GYSNP0043 |
| AGCCCAATCACAGAGAACCATTAAAAACTATAAGGTATT | | TGTTTACTGTAAAAATTTACACCAGCTTAGTTTTGTT[T/C]AAC | | | | | |
| CCGTCTCTCAGCTTACTTTTAGTTGCC_NODE_1_length_658_ | | TTTAATCATATATATATTTTTTTCTAATTTAATTATCCATTTTC | | | | | |
| cov_66.031914 | | AATAAAGTAACCCATTCTTATATCAGTTTCAAA | | | | | |
| RAD_kmer_0000181_1_TGCAGGTTCCTCTGTTAGAGCACAT | 356 | CACAATCAGAAGCTCTCTCTCATCTATTTTACATATCAACCTT | R | G | R | G | GYSNP0044 |
| GGCCTAACTATCGTATATGGAATCCCGCTTTCCCTAATCA | | TTTATCAAGTAAACCGCTCGTATTTTCCTCATCCTTG[G/A]TTG | | | | | |
| AATCCTCCCCCTGTTGACAAATTGAC_NODE_3_length_501_ | | TTTTTATATATCACCCTAGTCTTGACCAATTTTGAGCGAGATG | | | | | |
| cov_56.355289 | | TGCATGAGGCGCCACCTTAAGGTTCTGTGGCGT | | | | | |
| RAD_kmer_0000189_1_TGCAGGCGTTTACCAGTGCCTATGT | 448 | TTGATTTCAGTTGTCTTTTATTCCAGCTTATTTCAGGTT | A | A | W | W | GYSNP0045 |
| CATATAAAGCCACATGTTCTGAATACCCGCTGGCCAGAAA | | CGGACAACGGTTTGCTAAAACTGTATGATATTCAGCA[A/T]AT | | | | | |
| CAATTCATCCACTGAGTTGACATGGA_NODE_1_length_589_ | | TCCTTCAACAAACAACGGTGTTTACCCAAGTGGCGCCTCTGTT | | | | | |
| cov_128.200333 | | TCCTTTGATGAGTTTGACCAATTGACATCTGTCCA | | | | | |
| RAD_kmer_0000191_1_TGCAGGGTTTGAGAACTGGAAAATT | 431 | TCATCGTTTGATTGATCTTGCTCTTCATCCTGACCAATGACTA | C | Y | C | T | GYSNP0046 |
| TGGCGATCTGCAAAGCGGCAATTAGGATTTTCTTTGAGGA | | TGAACTCGTCATCCCGATTATTATTCAAAGTCAAAAC[C/T]GG | | | | | |
| AAGAATTGGAAGATAAATGGAAGCCA_NODE_1_length_507_ | | TTCATCACTTGGCTTCCATTTATCTTCCAATTCTTTCCTCAAAG | | | | | |
| cov_137.633133 | | AAAATCCTAATTGCCGCTTTGCAGATCGCCAAAT | | | | | |
| RAD_kmer_0000192_1_TGCAGGCTCATAAAATTACGTAAAC | 413 | AAGCGTACCATAATGTGACCCCTAGGTCTCATGCGCCAAACT | — | A | W | A | GYSNP0047 |
| GCCACGTGGTACTCTAGCGATGAGTTGCCAAATGCGTATG | | CACCGTCTCATGCACCAAACCCTCAACCTCCTATGCCA[A/T]T | | | | | |
| TGTGTGTGTGTATGTGTATATATGTA_NODE_1_length_537_ | | AGTTTCCATCTCTTGGGCCATATGGCGCAACACATAGGATCC | | | | | |
| cov_134.858475 | | TACCCTGTATATATGTACATATATACACATACACAC | | | | | |
| RAD_kmer_0000192_1_TGCAGGCTCATAAAATTACGTAAAC | 466 | GCACCAAACCCTCAACCTCCTATGCCAATAGTTTCCATCTCTT | — | T | K | T | GYSNP0048 |
| GCCACGTGGTACTCTAGCGATGAGTTGCCAAATGCGTATG | | GGGCCATATGGCGCAACACATAGGATCCTACCCTGTA[T/G]AT | | | | | |
| TGTGTGTGTGTATGTGTATATATGTA_NODE_1_length_537_ | | ATGTTACATATATACACATACACACACACACATACGCATTTG | | | | | |
| cov_134.858475 | | GCAACTCATCGCTAGAGTACCACGTGGCGTTTACGT | | | | | |
| RAD_kmer_0000194_1_TGCAGGAATATTATCCAGCAAATAG | 241 | AATGTCATGGTGTCCAGATTCTCAAAAGAATATGCTCGCTTT | W | T | W | T | GYSNP0049 |
| CTGTCATGCTTTTAGAGTTTCTCATGTTGATGT | | ACAACTAAAGGCAATGAATCACTTGATAGACCATTGTTGCTGGC[A/T]A | | | | | |
| ACCTGTCTTGACCCGTAACCAAACC_NODE_1_length_615_cov_ | | AATGTGCATATTGTTCCACCACATGGGCCTGTTTTTGAGATCC | | | | | |
| 77.027641 | | GGGTTCAGGATGGTTATGGTGCAAGATGGTCCTTAA | | | | | |
| RAD_kmer_0000194_1_TGCAGGAATATTATCCAGCAAATAG | 326 | GTGCATATTGTTCCACCACATGGGCCTGTTTTTGAGATCCGGG | G | A | G | A | GYSNP0050 |
| CTGTCATGCTTTTAGAGTTTCTCATGTTGATGTAAACAACT | | TTCAGGATGGTATGGTGCAAGATGGTCCTTAAACCC[G/A]AC | | | | | |
| ACCTGTCTTGACCCGTAACCAAACC_NODE_1_length_615_cov_ | | CAAGGTATGCATCATGCATGCACACACGATTTTAATGACTCT | | | | | |
| 77.027641 | | TGGTTTATCTCTTTTGTAATAATGTCCGGGCTTTCT | | | | | |
| RAD_kmer_0000195_1_TGCAGGAAGCTATGCTGATCTAATT | 153 | TGTGAAGCTTGTTGTATAGCTAGAAGTCGGCACCAGCACCAC | Y | T | Y | Y | GYSNP0051 |
| CACAGTCTCTCCATTGGAGTAATCAAATCAGATAGATAGT | | AAACACCTAAACAATCACCACAAATATCGAAAAGTTGT[T/C] | | | | | |
| GTGCATGTGTTTAGATATCAGATTTG_NODE_1_length_591_ | | ATCAACTTCTTCCCTACATAAAATGCATAAGAAAATTAGTAA | | | | | |
| cov_153.962769 | | TTTGGGGAAGTCAAAGGTATGTGCATGCGTAAATTACA | | | | | |
| RAD_kmer_0000198_1_TGCAGGTAACAATTTCGAACTTATT | 233 | AAAATTCGGAGGGTCGGGCGACCCCCTTCCCCCCAAGAAGCT | T | C | T | Y | GYSNP0052 |
| GATACATACCTCATGATGTTGGAGCGCTCTAGTAGTTGTA | | ACGCCAATGGGCCGATGAGTCTCTTCAAATTTCAACAA[T/C]C | | | | | |
| ACCCTGGTATGACTTGTATACAAACG_NODE_1_length_612_ | | CCTTTGTTTTGTTCGTTCGATCAGTGTGCTGAGCTTAAAGCTG | | | | | |
| cov_94.674835 | | CTGATATGTATAGTTAATTACTAAAATAGACCAC | | | | | |
| RAD_kmer_0000211_1_TGCAGGGTCTATATAGGTAAGATTA | 164 | ATAATTATGCACTCAAATTTCTAATATCTTACATCTATGACAC | A | A | G | — | GYSNP0053 |
| TGAATGCTTTACGTGATCCCAGGTTTATCAAGATCTGTGT | | CTAAAAGAATAAAAAATAGTAATGCGTCATCGGGTTG[G/A]G | | | | | |
| GTATGACTAACGGGCGGGTGGATGTT_NODE_1_length_231_ | | TCGGTCCCTAGTATCACTAGAATGTTGAATTACAAACCCTTTG | | | | | |
| cov_78.406929 | | ATGTAGGCTCAAGTTGAGTAGCTTCCAAAGAAAATA | | | | | |
| RAD_kmer_0000219_1_TGCAGGCGACGCGGCGTAGGACCG | 333 | ACAGAATAAAAGGCTAAATTGATTGTTTATGATGCAACGACA | — | A | C | M | GYSNP0054 |
| AAGTCCAATAAGCGGCTGACTGAAGTCAGCGGCAATAAA | | CCACCTCAGCCGCTATGCCAGGCACACATCGTATTCCT[C/A]A | | | | | |
| AGCAAAACCGTAGTCCAAGAGGCAGACC_NODE_1_length_ | | AGTTGTTCCTTGCTCTCATGCCTCCACTCTTTTTGTTTTTTTCT | | | | | |
| 460_cov_92.858696 | | TTTATTGTGCCGACTTCGGTCTGCCTCTTGGACTA | | | | | |
| RAD_kmer_0000227_1_TGCAGGTACAAAACATATTCTATTA | 227 | CCCCCATACCGCCCACCGCCATCAGTCAATCATCTACTTTTCT | G | T | T | T | GYSNP0055 |
| ACAAATTAATGATGCTTGAAAGTCTGATCCTTCTGAAAAA | | GAATCAATTCTCCAAATCACTTTGTAAGTTTCTGTA[T/G]CCA | | | | | |
| ACAAGATGTCTCAATTAAATAAACTC_NODE_1_length_415_ | | TCATATGTTTGTTGTCAATTAACAAAAAAATATATGTCAATCG | | | | | |
| cov_100.260239 | | CATGCAACTGGAATGATTAACAATATTGACAAAG | | | | | |
| RAD_kmer_0000229_1_TGCAGGATTTGAACTCCCAACCTTT | 225 | AGTTAAGGACCAATTAACTCTGGATCATGCGTAATGCGAGAA | G | G | A | G | GYSNP0056 |
| TGGTTGTGGAGTACATCACTTAACCATCAGGATGTCCCCT | | AACTGCCCAAAAACATATTTGTGTGTAATGCAAGGCGG[A/G] | | | | | |
| TGGGGACAGTAGTACATACCTATATC_NODE_1_length_545_ | | CACTCTTTTATGTTTAAAAGATATATGAGGTTATAACTTATAA | | | | | |
| cov_83.005501 | | GCATTTAAATGCCTTTGTTGTTGCAACATGCCTGGTG | | | | | |
| RAD_kmer_0000229_1_TGCAGGATTTGAACTCCCAACCTTT | 370 | TGCAACATGCCTGGTGAGGAGTCCAGTAGATCACCAAGAGCA | T | T | A | T | GYSNP0057 |
| TGGTTGTGGAGTACATCACTTAACCATCAGGATGTCCCCT | | ACTCTTAACTCAACAACTGTTAGACAATTGACAGCCAT[A/T]T | | | | | |
| TGGGGACAGTAGTACATACCTATATC_NODE_1_length_545_ | | TGTTAGGGGTTTCTTTAAGCTCACTTCTTTTGTGAGCGGATGC | | | | | |
| cov_83.005501 | | TAGTGAGCTGATATAGATTTGAACAATAAGATCACT | | | | | |
| RAD_kmer_0000235_1_TGCAGGGCGAGATTCAATCCACCAT | 259 | TCAGCTGACAAGAGCAAGTTTAAGGTTTGTAAATGGTTTCTC | T | Y | T | C | GYSNP0058 |
| TTTCTTCCTGATCAAAATACAAAAGCTGCACAAAGTTGAA | | TTGTTAAACACAACTTTGCCGAATATAATCTAATTT[T/C]A | | | | | |
| GACTTGGGATAAATACAGGACATTAT_NODE_1_length_587_ | | AAGTTATAATGTTGTATAGGTGTAGGTGTAAGGTTTGACCTA | | | | | |
| cov_73.243614 | | TTTACTAGTGAATGAGTTGATTCACGTTATTTTTATC | | | | | |
| RAD_kmer_0000235_1_TGCAGGGCGAGATTCAATCCACCAT | 327 | ATAATCTAATTTTAAAGTTATAATGTTGTATAGGTGTAGGTGT | C | Y | C | T | GYSNP0059 |
| TTTCTTCCTGATCAAAATACAAAAGCTGCACAAAGTTGAA | | AAGGTTTGACCTATTTACTAGTGAATCAGTTGATTCA[C/T]GT | | | | | |
| GACTTGGGATAAATACAGGACATTAT_NODE_1_length_587_ | | TATTTTTATCGCTAGCAGGTCAAAAGTTGCTCGAAGTGTAAT | | | | | |
| cov_73.243614 | | ATTAAACCTTTACTAAAAAAATATATATTGTTATTA | | | | | |
| RAD_kmer_0000239_1_TGCAGGCTTGTGATAGCGAAGAGG | 382 | TTTTATACATGAAAAAGATGAGCTGTTCGGAGGGAACCCTAG | W | T | W | T | GYSNP0060 |
| ACGGTTTATTTGGTTTTGACTTGCACAGTGATGGTGACGG | | GACCGAACCATTTGTACCCAGTTCATGATTTTTTTCGT[A/T]TT | | | | | |
| CAACAGGTGATAATTATTTCTTGAAAA_NODE_1_length_540_ | | CTTTGATTCCAGCATTCAGGTGCCTGTTGGGGAAGAGGGTTA | | | | | |
| cov_162.622223 | | TGAAAAAATTCGCATTTTGACAACATTTACAGCGAA | | | | | |
| RAD_kmer_0000240_1_TGCAGGGACGCTTGTACTTATTGCA | 432 | TAAACAATAAACAAAGTAGGGGTGGGCAAAGTTTGGTTAAA | G | A | G | R | GYSNP0061 |
| TTAAATGTAATACATATGACGATTATTAACCCATTTCTTTC | | ATCACATGATCTAAAACATGCTAACTTCAGATTTTGGTT[G/A] | | | | | |
| TCTCTTCAAGTTCGGTATAATGAAC_NODE_1_length_515_cov_ | | ATTTTTAGATTCGGTTAGGTTCATTATACCGAACTTGAAGAGA | | | | | |
| 105.728157 | | GAAAGAAATGGGTTAATAATCGTCATATGTATTACAT | | | | | |
| RAD_kmer_0000241_1_TGCAGGCTGCAACATATTATGATAA | 268 | AAAGGTAGATTTTATCTATATCCGAAAATTTAAGAGACAAAA | T | C | T | C | GYSNP0062 |
| AGATTTATGCGTACATATATTACATATGCATACGTATGTC | | CTAGTTGGAGTAAAATAGTGGTCCGAAAAGGGTAAACA[T/C] | | | | | |
| TGGATATATAGAAAGAATAATCCTCG_NODE_1_length_438_ | | AGTTCAGAAGCAAGCAAGCAAGGCTGACGCATTGCTTCTGGC | | | | | |
| cov_69.773972 | | ATCCTCTTCATTCATCTATATAAATCTATGGACAATAT | | | | | |
| RAD_kmer_0000243_1_TGCAGGTTTGACCATCTTTTGCTCG | 467 | CTGCATATAGTTTGATATATGTATTTCACTCGTTACTATGCGC | C | A | C | M | GYSNP0063 |
| AATTTCTTAATATTCGTTTATTATTATTATGAACTTTTGTT | | CTACGTAACCGAAAATTCCATTATAGCTAACAAAATT[C/A]AT | | | | | |
| GTGAATACACTAGTTACTTAAATTA_NODE_1_length_579_cov_ | | GCAACTTTTAGTCAAAACGTCTATGATCCGTTCTTCACGGCCA | | | | | |
| 112.160622 | | ATTAATTTAAGTAACTAGTGTATTCACAACAAAAG | | | | | |
| RAD_kmer_0000248_1_TGCAGGGTATGTATGTAATAATTGG | 117 | GCCTATAGTACATTTACCTCATTAGTCCACTTGATTTCTACTC | — | W | W | W | GYSNP0064 |
| ACCAATTGGGCTAGAGCCCTTAGTTAACAAAAGAACAAT | | ATTAGTTCACGTGTGCTGTTAAAAAACAAGAGTTTGA[A/T]TA | | | | | |
| ATTGATTAATGTAGGAGTTTGGTTGAT_NODE_1_length_553_ | | GTTTCATTGGTAGCACACTCACCTAATAAAACATAGACCAGG | | | | | |
| cov_106.108498 | | TTCTTTTTTCATAGAGAATATTTAAACACTTCTTTC | | | | | |
| RAD_kmer_0000251_1_TGCAGGAGCTAAAAGGAGAGGTCA | 125 | CTACAAGTAATTAACAACAATATAGTCATCAGAAGTTGCAAA | A | C | A | A | GYSNP0065 |
| ACACTTACCAGTTATAGTAGGTAAGCTCCCCCAGGACAGC | | AGTCATATGTGGTAAATTATATTTCACAAATGTCAAAA[A/C]T | | | | | |
| TCCTTAGGCGGGAACTCTTTTCCTATA_NODE_2_length_379_ | | CACCGACTGCCAATATAATTATTATCATGTCCGAAAGATAAA | | | | | |
| 16.308706 | | TTAAGAAAATAGATTAAATACAGATGTGACGTTAAAA | | | | | |
| RAD_kmer_0000254_1_TGCAGGCATACCTGAAACATAGCTT | 456 | TATATATTTCATGTTTAAAATATTTCGAACTTATCAATCATAT | A | W | W | A | GYSNP0066 |
| CTTCAACGTCCAAACACATTGTTTCAAAGTTTAGGTATGC | | GTTTGTTTACATCAATGCTTAACTGTGTTGTGTTTTT[A/T]TCC | | | | | |
| ATTACCAACCCATGCAATACCTTTAA_NODE_1_length_637_ | | TACGAAAACAACCTTTTAATAGAAAGAATCATTGTTTTTTCA | | | | | |
| cov_150.795914 | | GGTAGTATCTTGGTGTGTTATCAAGCAACTCAATA | | | | | |
| RAD_kmer_0000262_1_TGCAGGCAATGAAAAAATGAATGTT | 84 | AGTTCAAACGTGCAATTAACTCGTCGTGCCATGTTGGACAAA | Y | T | C | C | GYSNP0067 |
| TGCTTTAGCATAGATGCCAATGGTATTCTTGATGTATCGG | | CCAGAAATGTCCATCAAAAACAATACATACTGTCATAC[C/T]G | | | | | |
| CTAAGTTGGTATCTACTGGTAATGAA_NODE_1_length_471_ | | TGGCTATGATTATAGGATTATAAAAGTTCACAAAAAAAAAAA | | | | | |
| cov_37.197453 | | ATTAGTATTATAAATTAAGTAATCAAGTTACTTGCCT | | | | | |
| RAD_kmer_0000264_1_TGCAGGTAATTCAATCATGGGTTTA | 157 | TCCGTGAAAAATTACCAAAATAGTCAAAAAGTTAGACCAAA | M | M | C | M | GYSNP0068 |
| GTTTAAAAATTAGGACTGGCAAAACCGGTTCAATTATGTT | | GAATGTCAAAAAATAGACATAAAAATTTAGCCTTCACCA[C/A] | | | | | |
| AAACGGGTAGAGACGATGCTATTAAC_NODE_1_length_554_ | | AGTAGTCCCTCATTTCCTCCAATCAGAAGCCGCCACATCAGC | | | | | |
| cov_126.796028 | | TCTTGTTGGCTTACATGGTTAAGATTTAGCCTATGCTTT | | | | | |
| RAD_kmer_0000268_1_TGCAGGGTTAGGCTTCATCAATGGA | 143 | GGATTTGAAATTGCACACCAGCTTGGATTGCAAGGGTTAACC | G | G | A | G | GYSNP0069 |
| ATTGCAGCTTTGATCATATTCTTGGCCCCGGTATAATTGAT | | GTTGTTCTTACTTCAAGAGAGACTGCTGTAGGCGAAGA[A/G]T | | | | | |
| GGCTATAACTTTTTCAGCAAATTCA_NODE_1_length_471_Cov_ | | CAGCAAAAGTCCTACAAGAAGGAGGCTTAAAAGTGGTCTTCC | | | | | |
| 94.598724 | | ATCAACTCGACGTTATAGATCAACAATCGATTGATAC | | | | | |
| RAD_kmer_0000269_1_TGCAGGATACGATGCATAAAACTGA | 519 | TCGTTCTCAGCTCTCATTAGCTATGCATACAACAACAACACGT | G | T | G | G | GYSNP0070 |
| TCACCTCTAGAATAACCAGCTCTAGTCCTGCAAGAATTAT | | ACGTATACTGTTACGAGTCTGCTTTATAAGTAAATTT[G/T]TTA | | | | | |
| ATAAAGAGCAAAAGAGAAATGATATT_NODE_1_length_601_ | | TTCCTTGAAATCATAATATCATTTCTCTTTTGCTCTTTATATAA | | | | | |
| cov_100.224625 | | TTCTTGCAGGACTAGAGCTGGTTATTCTAGAGG | | | | | |
| RAD_kmer_0000271_1_TGCAGGTGGAATGGTGGCTTCAGAT | 396 | GTCGGCACCTAATATCAAGAATATTTGTCGCAGACCCTGCAC | K | G | G | T | GYSNP0071 |
| ATAATCTGCATGATCGTATCCACACTCTGCATCGGTTGAT | | AGGTTTAGTCATACAAAAATTTCACCTTCTATTGAGTG[G/T]T | | | | | |
| CAGGCTCTTCAAACTGGTTCATCATC_NODE_1_length_572_ | | ACTATGTGCTAATGATGTCGTACATTTTATCAGAGAATCACG | | | | | |
| cov_142.638107 | | ATGGACGAGATAAAGAAACACGAGTGGTTTCTAAAGA | | | | | |
| RAD_kmer_0000273_1_TGCAGGAGAGTTTTGACAAGATTAA | 327 | GTGCTGTCACTGTCAGTACTGTCGCTGTCAACATCTGACTCGA | G | K | T | K | GYSNP0072 |
| TAGGCAGCTAGAGAAGGTGACGAGGGTGACGGAATCTGA | | TATCTACTTCTTCTTGATCCTCAAAACTTTCAGGATT[T/G]TCT | | | | | |
| TAGGGCGCCAAATAATTATGTTAAGGC_NODE_1_length_548_ | | CTGCACTTATTAATCAAATCCTCATACTGCTTATTATTCTTCTT | | | | | |
| cov_139.049271 | | CAACTTCTGCCTCATCGCATTCAACGCCTTAGC | | | | | |
| RAD_kmer_0000282_1_TGCAGGCAGTGGATAACACAACCG | 157 | CTGGGTCGTGTTCAATGCGTTCAGTTATAATCTAAAAGTTTGA | T | — | A | A | GYSNP0073 |
| GATTTTCAAATTTTATGTAATTTTTTCCCTTCCAAAATCTC | | ATAAAGCGCTTTAGGAGATTGTATCCATTAAAAATAG[A/T]GT | | | | | |
| ATTTACCAAGACTCAAAACTTTGTTA_NODE_1_length_617_ | | TTAGATGAGTTTTAACCCATTTGACCCGTTTGAGTTAAAACAC | | | | | |
| cov_118.309563 | | AATCAAAATCGACCCACTATAAGTAAATCAGTCAA | | | | | |
| RAD_kmer_0000287_1_TGCAGGATACATGCAGTGGTTGTAA | 132 | GTCTATTTGTATTTAATTTTATACATTCTACTATTGTATGCGTG | T | C | C | Y | GYSNP0074 |
| AAATGGCTTCTAAAAGTTTACAAACAAATGCTCGCACATT | | TAACACTTTACTCAAATAAGTTGACTACCATATAAA[C/T]TTT | | | | | |
| CTACAATTAATCATGCTATTGTTTGA_NODE_2_length_585_ | | GAACTTAAACCCTTTCCTCTTTGATCATCCAAGTCAAAGTGCA | | | | | |
| cov_89.678635 | | TCCTTCTCTGTTGCAAATGGAGTATAATATATAT | | | | | |
| RAD_kmer_0000294_1_TGCAGGAGCCTATCAAAGCACAGGT | 284 | TGGCCTCCAAGATGATGGCGCACTAGACGCCGACACTTGCAG | G | S | G | S | GYSNP0075 |
| GCTCCTCATCAGCCTATTTCATCATTTCAGAATGCAGGGT | | GCTGTTTATAGCAGAATAGCCACTGGAAACAGCAGGAG[G/C] | | | | | |
| CTTATGCTGGTCCCGCCAGTTATACA_NODE_1_length_446_ | | AGCAGGCACCAAATTGGAAGCGCTGGTAGAAGCTACATTTTC | | | | | |
| cov_37.605381 | | CGTTCCGGGAGCACAAGTAACCTCCGTTTGACTATAAT | | | | | |
| RAD_kmer_0000297_1_TGCAGGACTAAGAATTTTGTTTATT | 342 | TAAGATCATTCTTCGGCTCTTTCCAGCAAAAAAAGAGTTGA | T | G | G | T | GYSNP0076 |
| CCATCAACAAGACCATGATTATCCTTTGGAGCTTTGCTG | | ATTTGCAATGATGCTATAACTTCACCTAAGGTGGAGA[G/T]T | | | | | |
| TCTTCTTGGACTTCTGCCCATTTATT_NODE_1_length_417_cov_ | | ATATCTACTTCTGGTGCAGGTAAAGCTTGTTATAATTTTTTTT | | | | | |
| 33.621101 | | TGTTTGTTATTGAAATAGGGATTTGGTACTTAAGGT | | | | | |
| RAD_kmer_0000297_1_TGCAGGACTAAGAATTTTGTTTATT | 84 | GCTTGCTACAGTTGCTTATGTTCAGCTAGTATTGGATTATAAT | A | A | C | A | GYSNP0077 |
| CCATCAACAAGACCATGATTATCCTTTGCAGCTTTTGCTG | | GTATTATCAAATTAAATGAATATCTTCATTTTCTATG[C/A]TTT | | | | | |
| TCTTCTTGGACTTCTGCCCATTTATT_NODE_3_length_191_cov_ | | AGGTCATAATACAAGTTCGAAGACCGGAGCTAATGTTAAAAT | | | | | |
| 283.151825 | | AAATGGGCAGAAGTCCAAGAAGACAGCAAAAGCTC | | | | | |
| RAD_kmer_0000299_1_TGCAGGTGTGGATTTTAAGGTGAAA | 372 | GGGTACACTTTGGTAAATAGTGTATCTTTAACAACGCCCAA | T | A | W | A | GYSNP0078 |
| TATGTCACCATTGATGGGAAGAAGTTGAAGCTTGCTATTT | | ACAAGATCATAACATGTAATACAACAAATAACTTATCT[T/A]T | | | | | |
| GGGATACTGGTAACTGTCTTTATTGC_NODE_1_length_442_ | | TAAAGCAATAAAGACAGTTACCAGTATCCCAAATAGCAAGCT | | | | | |
| cov_198.454758 | | TCAACTTCTTCCGATCAATGGTGACATATTTCACCTT | | | | | |
| RAD_kmer_0000300_1_TGCAGGATTTGAATCCATAGCTAGA | 102 | TTCCTTGAGATATGCGTTGAACAGCAGAAGACCAATGAGGAT | R | A | R | R | GYSNP0079 |
| GGTCTTACAAATGCAAAACTTAGAGTCGACTTCCGTCATC | | TCCACATGTAGGGTCCACCTACAGCTTGAAGGGCAACA[G/A] | | | | | |
| GTCTTCTATCTTTATGCTCAGATGTG_NODE_1_length_638_cov_ | | CACTTGTGTTCCCTCCATTGTTCAATGTTGTGGAGTTTGGCTT | | | | | |
| 125.835426 | | CACTGGGGTTGGAGATTTCAGTACAGGTGGGGCCAAG | | | | | |
| RAD_kmer_0000303_1_TGCAGGTGGCTTGGGTTTATTTGAG | 245 | CAGTGAACTGTTCGTGAACAGTTCAATGGAAAGTTCGTTTAT | A | T | A | A | GYSNP0080 |
| GTGGGTTTCATCAAAAAAACAAGCAAATCCCACCAGTGTT | | GTTTCTTCATTTAAGAAATAAACGAATATCATATGAGC[A/T]C | | | | | |
| GCAGTGGTTGGCAAATGGGTGTGTAA_NODE_1_length_642_ | | ATATTTTTCTTCATCTGATATATTAACAAGCATGAACATGGAT | | | | | |
| cov_47.507790 | | TTTTGCTGATTTAACTAAGGGGGTGTTTGTTTTTTG | | | | | |
| RAD_kmer_0000303_1_TGCAGGTGGCTTGGGTTTATTTGAG | 295 | ATTTAAGAAATAAACGAATATCATATGAGCACATATTTTTCTT | T | C | C | T | GYSNP0081 |
| GTGGGTTTCATCAAAAAAACAAGCAAATCCCACCAGTGTT | | CATCTGATATATTAACAAGCATGAACATGGATTTTTG[C/T]TG | | | | | |
| GCAGTGGTTGGCAAATGGGTGTGTAA_NODE_1_length_642_ | | ATTTAACTAAGGGGGTGTTTGTTTTTTGCCAGAAGAGCTTCCT | | | | | |
| cov_47.507790 | | GGCCTCTTATGTCTGCGCCGCGCAGACCATGCGCA | | | | | |
| RAD_kmer_0000313_1_TGCAGGAAATCATCAAACGTAGATG | 363 | TTTTTCTACCATAATGTTATATGAGATGAGTTGGGAATGTGAA | C | T | C | C | GYSNP0082 |
| ATAGGCATTCAATTCAAGGTACATAGAGGGATAAATAAG | | ATATGAAACTGATTTTCTGAGTATTGCGATTTGAAGT[C/T]GC | | | | | |
| TAATTTTAATTTCTATTGCAGCTGCTA_NODE_1_length_502_ | | GATTTGAAGTCGCCATATCATTTTAGTGATCAAATAAAATGA | | | | | |
| cov_107.324699 | | TTTTGTTAAGGGGTCAAATAGTCACTTTTTTAGCAG | | | | | |
| RAD_kmer_0000319_1_TGCAGGCGGTCATAGAGATGAAGA | 282 | TTTGGTAACAACTAGTAATGTTCATTTCAGAACTTCTTTGGTT | W | W | T | W | GYSNP0083 |
| GCTGAACCCGCAGCACATTGATCAAGTGGCGGTTCACACT | | AAAATCTTAGTTGATGATTTTCCCCTGTTTTTTTTTT[T/A]AAG | | | | | |
| CACTCGACTCATGGACATGCTCATGGA_NODE_1_length_450_ | | TCATTTGTGTTGTGATTGGGCAGCTGTTTTTTTATGCTAGAG | | | | | |
| cov_50.740002 | | CAATAGTCAAATGATTCTTCACAATTTAAGTGGA | | | | | |
| RAD_kmer_0000320_1_TGCAGGGTGTGCTGCATGATTTCTT | 235 | TTAAAGACCTTCATTGAGTATCAGGTTAAAGATGAACATATT | G | A | A | A | GYSNP0084 |
| CTCCCGTTTGGGTATTATATATTGATCTTCTTTGTATTAAG | | TACCGTTCAAGGGAAAGGCCCCAAGCAATAACCGTAAC[A/G] | | | | | |
| GGGCACTATCATAATTCGCTATTAA_NODE_1_length_607_cov_ | | TCCTCAGGAAGTCCCATAGGAAGTAACATTTCAGGTCTGAAC | | | | | |
| 144.090607 | | ATTCCAGAATTTCCTACCTCAACCCATTTCTTAAGGCC | | | | | |
| RAD_kmer_0000326_1_TGCAGGAAAGACAGCCTCACCAAA | 130 | TAGGCATTTAAAGATGGCCAACGTGGTACAAAGCTTCCTGGA | C | C | — | C | GYSNP0085 |
| GTTAGACTTTTGCCATTTTTTTTAACTTCATAGTTTATAGC | | GTGTTGAAAACACTAGTTGGAGTTGTGTATGCAGACAC[A/C]A | | | | | |
| TTACAAGTCTTTACCGGTTCAAGAGT_NODE_1_length_629_ | | GCACAAAGAAACTCCATAGCTGTAAGTTATCTGTTTAAGTGG | | | | | |
| cov_74.640701 | | TAAAATAGGTGTGGCTCGGACAACAGGTATGACAGGT | | | | | |
| RAD_kmer_0000328_1_TGCAGGCTCATTTGAACACATAGAT | 109 | AATTAAACCTATAAATTTCTATATTTGCTAGATACCTTTTGTA | — | C | Y | T | GYSNP0086 |
| TATGGTGCAAAATAAAACGGGTCAAAAGTAGCCAAACCT | | TTTTTTGTATCTAATATATGCTCATAGATTATTGTTTGTTTG[T/C]TCA | | | | | |
| ATCCTTAGCCATGTTTCCTAATGTTGC_NODE_4_length_242_ | | AAACATAGGCCCTACTTTGTGTTGACCATTGTTATGTGGAAG | | | | | |
| cov_11.285124 | | CACAAAAGAATGCATATGGAAGCGCAAAAGAATGC | | | | | |
| RAD_kmer_0000329_1_TGCAGGTGAAATCATACATGTATAT | 140 | AGTTAATGGTGTACTAATTATTTGTTTGCTAGACAATAGAAA | T | Y | Y | Y | GYSNP0087 |
| GCAAGGATTCCAAATCCCTGTCTTAACTTGACAATTAAGG | | CGATTGTACTCATTTCTTCAGCCATTATGTTTCACTGT[C/T]GG | | | | | |
| AGCCACTGCGTGAGGATTCTAATTTG_NODE_1_length_579_ | | GTGATGTCACTATGACTATATATATCTCATGGTTTGCCTATGC | | | | | |
| cov_95.576859 | | TATTATAATGTTTATATATAATTTATCCTTTTTAA | | | | | |
| RAD_kmer_0000334_1_TGCAGGTCTCTTACCATCAGAACTA | 548 | GCCGGTCAGCAACAAGAACAGGTTAAGAGTAGGGGTTCGAG | T | C | C | Y | GYSNP0088 |
| ACAAACCGTACAAAATCAAATTTGAGAGCATAAGGCAAC | | TCGGGGGGTTTGATCAATTTATAGATGCATACACCGACC[C/T] | | | | | |
| TCTCCAACGACCGTGTTAAAAACATCG_NODE_1_length_649_ | | GGAAACCCGCCAGGTTACTATGACAGGTTTCTGTGTCGATGT | | | | | |
| cov_119.767334 | | TTTTAACACGGTCGTTGGAGAGTTGCCTTATGCTCTCA | | | | | |
| RAD_kmer_0000336_1_TGCAGGCGCCTTGGGCTGAAAGAA | 456 | GATAAAGGTTGACATCGATGATTACATGGTTCGACAACTGGA | T | T | G | G | GYSNP0089 |
| AGCATTCAAGTAAAACTTTAGCAACTTAACGACTAATAAG | | CGGGAATAAAAATGAGTGGGGTTGGTGCAAGCAAAAAG[G/T] | | | | | |
| TAGTAAAATTATGACAAGTAGCAAGAA_NODE_1_length_559_ | | TATAGTTTTGAATTCTGGTATATAACTCGACTTTATGCTTCTT | | | | | |
| cov_168.334518 | | GCTACTTGTCATAATTTTACTACTTATTAGTCGTTAA | | | | | |
| RAD_kmer_0000339_1_TGCAGGTAATAAAAAAATCATTTAA | 158 | ATACACTTGCTCCACCGACCACATCCCGAGCTTCGATCAAGT | C | T | T | T | GYSNP0090 |
| CTTACTTTATACACACAAACATAACAATAGCAATGTTGCT | | TTCATGAAGGTTCATTTCAGTTCATGAATTTGGCATTT[T/C]CA | | | | | |
| TCACCTCATTTTACTTTCAGTTATAC_NODE_1_length_446_cov_ | | CTGATTTATATTGGAATTAAATATATTCATTTACTATTGTTGT | | | | | |
| 116.127800 | | ATTTTGAGTGAGCTTTCGGTATGCATAGTTTTTTC | | | | | |
| RAD_kmer_0000339_1_TGCAGGTAATAAAAAAATCATTTAA | 247 | TATATTGGAATTAAATATTCATTTACTATTGTTGTATTTG | G | T | K | G | GYSNP0091 |
| CTTACTTTATACACACAAACATAACAATAGCAATGTTGCT | | AGTGAGCTTTCGGTATGCATAGTTTTTTCTTCGTTTT[G/T]TAG | | | | | |
| TCACCTCATTTTACTTTCAGTTATAC_NODE_1_length_446_cov_ | | AAATGGGGCTCTAATATGCTTGATGCAGTTATATTCTAAATC | | | | | |
| 116_127800 | | ACTGGTAGTTGGAATACGTGTGTAGAGTTTACCTA | | | | | |
| RAD_kmer_0000340_1_TGCAGGGAATTAAACAACAAATTA | 259 | GATGATGATGATGATGAAGACTTTCTTAGGAAATGTTGCAGT | W | T | W | T | GYSNP0092 |
| AAAAAAACAAAAAACAAATAGAACAAAGCTTATTAATTT | | GATGATGATTTGTTTGTTGTCTTCGATCATAGTCTTAT[T/A]AA | | | | | |
| ATTGACAACAGTAACAATTGATTTATG_NODE_1_length_584_ | | TAATGTGAGTCAAGATCCTTATGTATGTTTCATGAGGATCAA | | | | | |
| cov_150.150681 | | GGAGTTGCTATGTAGTAGAATTTGTTGAAAAACATT | | | | | |
| RAD_kmer_0000348_1_TGCAGGGTTAGGCTTCATCTGGA | 206 | TAATGTATAAACTGTAGGTGGTGGTCTGCTGAAACAATAGCT | Y | Y | T | T | GYSNP0093 |
| ATTGCAGCTTTGATCATATTCTTGGCTCCGGTATAATTGAT | | GTGGTGACTGGTGCAAATCGAGGAATCGGATTTGAAAT[T/C] | | | | | |
| GGCTATAACTTTTTCAGCAAATTCA_NODE_1_length_402_cov_ | | GCACACCAGCTTGGATTGCAAGGGTTAACCGTTGTTCTTACTT | | | | | |
| 46.679104 | | CAAGAGAGACTGCTGTAGGTGAAGAATCAGCAAAGGT | | | | | |
| RAD_kmer_0000350_1_TGCAGGATGCACCTATAAAACCGAA | 210 | TTTCCTTATAATTTATTTTAAATATCAATACTTTATTATAAGTT | G | K | K | G | GYSNP0094 |
| ATTCATACACTAACAAGATTAAACATACCAAAAGCATTGG | | AATACAATGCAGTATCTGATTAGGTTAGGGTTAGTA[G/T]CTT | | | | | |
| CAGATCTTAGTTGGGCCAGTTTTTTT_NODE_2_length_445_cov_ | | TTAGAGAAGATTAATGTATACTCTTGTTAGTTTAAAGTTAAAT | | | | | |
| 53.757305 | | ATGTTATAAACCTCTTCATTGAATAATAATATTG | | | | | |
| RAD_kmer_0000363_1_TGCAGGAGGACTAACCATGTTAGAA | 376 | ACACCTTCGATTATTGTATTCTTCATAGATCTTCATCTAGTGT | — | C | Y | C | GYSNP0095 |
| CCTAAGTCATGTTATTCCATAAAGGCTTGTAAATAATTAA | | GGTTAGATCACGTGATTGTATCAATCACTCTTTTAGA[C/T]CA | | | | | |
| ATGCAAATTTATACACCAATTTCATT_NODE_1_length_585_ | | TTAAAAATGCTCGGTCTGCTTATATTTCATCATCCACTTAATA | | | | | |
| cov_74.617096 | | TGTGTCATTTGAGCTTAATATTAGCGTTTCAAATA | | | | | |
| RAD_kmer_0000363_1_TGCAGGAGGACTAACCATGTTAGAA | 482 | ATTTCATCATCCACTTAATATGTGTCATTTGAGCTTAATATTA | — | T | Y | T | GYSNP0096 |
| CCTAAGTCATGTTATTCCATAAAGGCTTGTAAATAATTAA | | GCGTTTCAAATATTCGATAATAGTACTAACATGGTAT[C/T]TA | | | | | |
| ATGCAAATTTATACACCAATTTCATT_NODE_1_length_585_ | | AAAGTTATTACTTATTACGCGTTTATCTGTAAAATGAATGAA | | | | | |
| cov_74.617096 | | ATTGGTGTATAAATTTGCATTTAATTATTTACAAGC | | | | | |
| RAD_kmer_0000366_1_TGCAGGTTGACCATATGCTTGAGTT | 354 | TAACTGATGCAATTTTGATCTGTTTGTTTGGTTTTAGCATCAT | A | W | A | W | GYSNP0097 |
| TGACTAGGTTGCTGTTGAGGGTATGCTTGATTCAGCTGCT | | ACTGTTGATCCGAGCCATGCACAAACACCATCTTATT[A/T]TG | | | | | |
| GCTGATAGCTGCCATAGTAACTTGAA_NODE_1_length_614_ | | CCCCACCTCTTGGCTCTGAACCAGCATCCTGGACCAGCTACA | | | | | |
| cov_166.032578 | | ATCATGCTCAGCAGACAGAAGCCGTGCCACCTGGCT | | | | | |
| RAD_kmer_0000368_1_TGCAGGAATGAAATGTGATGACTTG | 121 | TGAATGTTTGTCTTTTATGTATTTCGTAACTCATAGATTTATAT | A | A | W | W | GYSNP0098 |
| CTTCTTTTTGCCCATAAAACAAGCAAGTCTAGTATGATAG | | AGAGTAAATACAATGCAAGATAATTATGAAAGTCTA[T/A]AA | | | | | |
| TGTTCTGAAATAAAAGAAAGAATAAA_NODE_1_length_543_ | | ATTAATGTACAGGCTACAGATAAGCACAATAAATAGAGATA | | | | | |
| cov_126.263351 | | GTTAACTATTATTAATGTATCTCTAAGAAGATGATAA | | | | | |
| RAD_kmer_0000308_1_TGCAGGAATGAAATGTGATGACTTG | 228 | CACAATAAATAGAGATAGTTAACTATTATTAATGTATCTCTA | A | W | W | T | GYSNP0099 |
| CTTCTTTTTGCCCATAAAACAAGCAAGTCTAGTATGATAG | | AGAAGATGATAAGAACACGCATATATAATTATAAATAT[T/A] | | | | | |
| TGTTCTGAAATAAAAGAAAGAATAAA_NODE_1_length_543_ | | GCAGAATACACACACATTTTATATCCATTTATGTCATGATTAC | | | | | |
| cov_126.263351 | | CATGCTAGTTATGGAATTTGAATAACAGTAAACACTT | | | | | |
| RAD_kmer_0000371_1_TGCAGGAGCGTAAGCTGAGCGCCTT | 265 | ATGTAATTAACGTTATGATTTGCTGCATATGGAGTCTTGTTTA | W | W | T | T | GYSNP0100 |
| GATCGGGATTGTTGCTGAGGTGTTGGCTGAATAATGAAAA | | TCTAGAAGTCGGTTAGATCTTAAAGATCTATAAAATC[T/A]TA | | | | | |
| AATGTTCATAAGTATTTCATGTGTAG_NODE_1_length_660_ | | ATAAAATCATGAACAGCCCTGTAAATGGAAGAACTCTGCTAA | | | | | |
| cov_101.178787 | | ATCACATTAACTTTGTGCTTCAATGGTTTAATGAGA | | | | | |
| RAD_kmer_0000372_1_TGCAGGAAGATATATCTCTTGGATA | 152 | TATAGATGACTAACATTAGCTAAAAAAGAATGTAACATTAAC | W | W | W | T | GYSNP0101 |
| TTTGAGGTAAGTTTTTTATCCAGTTGTATAAACATATGATG | | AATAGAGGGCTTAACAATATTGATTAAAAATTCGAACA[T/A] | | | | | |
| CTCTATATTTGAACGAGATTTAGTT_NODE_1_length_588_cov_ | | AGAGATACTAATTTCCAAGGAAATAAAATTTACATAAAGTTA | | | | | |
| 139.896255 | | AAACAACTCAAGGGGCTGGAAGATGATGTTGGATCGTA | | | | | |
| RAD_kmer_0000375_1_TGCAGGCTTACACAATTACAGCATA | 413 | TGAATTTTTGAAGTTTGAACTACCAGAAGGGGAAAAACCTAG | K | T | T | K | GYSNP0102 |
| GTAACAATGAACTACTGATCGAATCTAAAACATGAAACTT | | GGTTTCTGCAACATTCACGTTAATCAGGTATTTGTTTC[T/G]TA | | | | | |
| GTAGTACAATATCTGGATTAAAAA_NODE_1_length_510_ | | ATTCTTTTGCTTTTCAATCCAGTTATTATGCACAATAACTTTC | | | | | |
| cov_46.274509 | | ATGTTCCAGATTCCATTAGATAATTCCGTTATCAT | | | | | |
| RAD_kmer_0000379_1_TGCAGGTGCAACACATTAGACAAAT | 177 | GCTTGCTCCTCATGGCCCAGTTTGTTACTGATTCGGATCGTGA | C | C | Y | Y | GYSNP0103 |
| CACAACGTATTTATCTTCACATAGGAAGGAAAACAAATA | | ACTAGTCGTTGCAAATGTATACCATTATATTAGCATC[C/T]AT | | | | | |
| GCAATCTTGCTTGCCGGAACAAATCCA_NODE_1_length_619_ | | TGTCAACTTTGTTGATAGCTCATAACCCTCTACCATAGAAACA | | | | | |
| cov_106.623589 | | CAACTGATGCTATCATATTCTTGGCCTTCATTATA | | | | | |
| RAD_kmer_0000385_1_TGCAGGTGATGGTCTTTTTACTATTG | 108 | GCCATATTTCGTGAGAAGTGTAAGAAGGATTAAAAACTGATA | T | T | G | K | GYSNP0104 |
| AAATGGAAAAAAAACATTACGAAAAGAAAACATTGCATA | | TGTAGACATCATGTTTTGGATTTAGGCATGATGTTTTA[G/T]GT | | | | | |
| AGATGATTGATCCTGATCTAAGAAAT_NODE_1_length_623_ | | TGTTTTGGCTAGAAAAATACCTAACACAACAATTTGAAACAC | | | | | |
| cov_87.775284 | | AATGTAATGGAGAAAATTATCATAAAATACGTTTGA | | | | | |
| RAD_kmer_000385_1_TGCAGGTGATGGTCTTTTTACTATTG | 209 | ACCTAACACAACAATTTGAAACACAATGTAATGGAGAAAATT | G | G | T | G | GYSNP0105 |
| AAATGGAAAAAAAACATTACGAAAAGAAAACATTGCATA | | ATGATAAAATACGTTTGATTGTTTGATGACCGATTTTA[T/G]T | | | | | |
| AGATGATTGATCCTGATCTAAGAAAT_NODE_1_length_623_ | | CTTGTTTCGCTACGGGAAATTTTGGTGTTGCCCTTGCAGGTGG | | | | | |
| cov_87.775284 | | AAAGGTTTTTTCCTTTAAGAGCATCACATAGACAAT | | | | | |
| RAD_kmer_0000386_1_TGCAGGTACCACACTTGCCTTTTGA | 331 | CACAATTTATGCGTACGAGAAAAAGTTAGAATAAAAGAAGA | G | R | R | G | GYSNP0106 |
| AGTCCTACAACCGTGATTATGTTTGTAGTTGGAAGGACAA | | AAAATTTGTAGAAATGCAGTTATTCGTCTGCATTTAACC[G/A] | | | | | |
| AATGCAGTGAATATGGCTTTCGTAAA_NODE_4_length_427_ | | AAGATATATGAATAAAATTAGTAAACAGAACAAGATATATTA | | | | | |
| cov_5.480093 | | GTTGTTATGATGCCTGGTTTAGAAACTACGGTTTTAAG | | | | | |
| RAD_kmer_0000394_1_TGCAGGAATTTAAAACTACCAGA | 200 | TAAGAAATGATTTCAACACTGTCATCACCATTACCCTGCAAT | T | A | T | T | GYSNP0107 |
| TTTAAACATTTATTTACAAATCATCAATTATCAACAAAAT | | AAAACACATATATAGCAAACATGTTAATATAAAATATA[T/A] | | | | | |
| TCCTAATTTAAAGGGGTGAAAACTTC_NODE_1_length_453_ | | AAAAAGGTAAAACATTAAAAAAAATGCAACCTTAGACGCA | | | | | |
| cov_55.388519 | | ACCTTAGAGGCTTGTACGACCATCTGTTTTTTCGAATCA | | | | | |
| RAD_kmer_0000394_1_TGCAGGAAAATTTAAAACTACCAGA | 275 | ATATATAAAAAAGGTAAAACATTAAAAAAAATGCAACCTTA | R | G | R | G | GYSNP0108 |
| TTTAAACATTTATTTACAAATCATCAATTATCAACAAAAT | | GACGCAACCTTAGAGGCTTGTACGACCATCTGTTTTTTC[G/A] | | | | | |
| TCCTAATTTAAAGGGGTGAAAACTTC_NODE_1_length_453_ | | AATCATTATGTTCTTCATCAACTGGAAGCTGTAAAATGTTAA | | | | | |
| cov_55.388519 | | ATGCACCGATTATACAATGAACTAAACATCACAGGAAA | | | | | |
| RAD_kmer_0000394_1_TGCAGGAAAATTTAAAACTACCAGA | 331 | GCTTGTACGACCATCTGTTTTTTCGAATCATTATGTTCTTCATC | R | G | R | — | GYSNP0109 |
| TTTAAACATTTATTTACAAATCATCAATTATCAACAAAAT | | AACTGGAAGCTGTAAAATGTTAAATGCACCGATTAT[A/G]CA | | | | | |
| TCCTAATTTAAAGGGGTGAAAACTTC_NODE_1_length_453_ | | ATGAACTAAACATCACAGGAAACTATCATATAATGATGCAAA | | | | | |
| cov_55.388519 | | TGTATAAAAAATACAAACCTTAGAAGCCTCAACCTT | | | | | |
| RAD_kmer_0000395_1_TGCAGGGTAGATTGCTTCTTAATAC | 203 | TTGATAATTTAGTGCTTATGCTCTGCCTTGTTTTCTGGGCATA | G | Y | G | Y | GYSNP0110 |
| AACTAATATTAAACAAAAACTTGTAAATATATTATTATTA | | TCATTTTGTGTTCATCCCATTTGGTTAGAATGATTTC[G/T]TG | | | | | |
| ACAAAACTAGAAGAACCTACTTTCAC_NODE_1_length_466_ | | TGTTGTGAACTAAATCTTTTACCATTTAGAACTAAGTAAATGT | | | | | |
| cov_151.302582 | | TTTACTGGTTCAGGTGATCAAACTGTCACGTTTAG | | | | | |
| RAD_kmer_0000398_1_TGCAGGCTTCGAGGAATGTCAACAG | 206 | TCATTTATGCGAATCTTGTATCAAAGATATCTTTCAATTTTGT | A | R | G | A | GYSNP0111 |
| CCATTGTACCCGCAGTGAAGAACCTGCAACTGATCGGAG | | TTTCTAAGATGTAGCAGGATATATGAGGTATTTTTGT[G/A]GT | | | | | |
| AAATAAAGGAAAAGACACCGGAAGAAC_NODE_1_length_557_ | | TGTTTGATGGATGAAGCTTTAATCGGGTAGTAGCAAACGGAT | | | | | |
| cov_76.549370 | | GTAGTTCGGTTCGCGTTTATTTGTAGCTAAATTATG | | | | | |
| RAD_kmer_0000399_1_TGCAGGCTACTATGAACCATTCACG | 125 | ATTCAAGAAACTAGCAAGTTGTGAAGGGTTAACGATGCCCGG | — | Y | Y | T | GYSNP0112 |
| CAGAAAATCAATGCCACATAAGCGACAAGGGAGAGATGA | | TATATGCACATCGCTACTCTTGAGGAGCTCAGACACCT[T/C]G | | | | | |
| AGAGTTTGACTTCTTGATGCCATCAAC_NODE_1_length_491_ | | CGGTACCTTTGATCTTCAGCATGAGCCTCAAGCATGCTCATAT | | | | | |
| cov_86.338089 | | TTTGCTCGGTTCTTGGCCCTTTTTGAGCAATGTCTT | | | | | |
| RAD_kmer_0000409_1_TGCAGGAAATGTTGAATGTGCCACC | 189 | TTGAATTTTTACTTATACTTTTTCGAGCAGTGTCCACAAGTTT | W | A | W | A | GYSNP0113 |
| TGTGTATATCGTTGGAGCTTTCATTCCAGCAACCATGATT | | ACGTCTTAGAAGCTGTTTCACAAGGAAAAAAAGTTAT[T/A]CT | | | | | |
| GCAGTGCTTTACTATTTTGATCATAG_NODE_1_length_563_ | | TTAAAAATCTTGGTTTAATTTGAAAACTAATATGCACCTGGTG | | | | | |
| cov_156.776199 | | TTTCAAAGTGGCTAAGCTCTTAGTGTGCATTGGAG | | | | | |
| RAD_kmer_0000410_1_TGCAGGAATGTTTTTCCTTTGATAG | 374 | ACAGCATTACATTCCATTATCTTTTTTTGGCTGGCCTGCAGAT | A | G | R | G | GYSNP0114 |
| GCAAGGGGTGTTTTTTTTTTTACTTCATTCCATCATGGGAT | | GGAACGTGTTTTAATTTAAATTTTTAGTATATTTATG[A/G]ATA | | | | | |
| GAAAGATACAGTATAACCCGTTCTC_NODE_1_length_581_cov_ | | GAGTGTATAGTTTTAACTCTACTTAATATTTCATTTTATAAAA | | | | | |
| 72.512909 | | TTTAATTATCTTTTATGTATAATAATTTAAATAC | | | | | |
| RAD_kmer_0000413_1_TGCAGGACTTATTTTGTATAGCAAC | 500 | TGGTGAAGCAATGTTGACAAATGAAAAATTTGGATCTAGTGA | C | C | C | A | GYSNP0115 |
| AGATCAACCTTTTCGGTTCCCTTCCTCCTTCGCTTTTCTAA | | GTATCCAATTCCTGGAAGGAGACACAACTGCACTCATA[C/A] | | | | | |
| TTAGAGCATTTTCCACTCTTGAAGG_NODE_1_length_651_cov_ | | ATACATCTACGCATATACATATGCCCACACGGACAAGTCTTT | | | | | |
| 100.402458 | | GCGTGCGTACACACATATATACATAAATACATAATTTC | | | | | |
| RAD_kmer_0000417_1_TGCAGGTCGGGCAAGGGGTTAGAT | 513 | AACACAATAAAAATTAGCCAAAAAAGAAACAAGTCAAACGA | M | A | A | M | GYSNP0116 |
| GGCCAATCAATGCAGGTTATTTTAATATGACCGAAACACT | | GTTGAAAGTAAAACAAAGTGTTTATGCATAAATCTCTTA[A/C] | | | | | |
| TTTTGTCCAAAACATTTCGTTTTTCTT_NODE_1_length_635_ | | AGCTTACATTTATAAGTTTTAGTATATGTTACTTGGCACCCTA | | | | | |
| cov_88.711807 | | GTTACTAGAAAGGAAAGAAAAACGAAATGTTTTGGAC | | | | | |
| RAD_kmer_0000420_1_TGCAGGGGTTTACAGAACAAGTGCA | 309 | CAGTTATGTGTATAAGCAAAATCACAAATACATACACACAAA | K | — | G | G | GYSNP0117 |
| GGTGCTCCCCAGCTTGTTAGAGTTCCATCCGGGTATCAAA | | CCAATACTAGAATACATTTATAAAAAAGGCCCTATTCG[G/T]C | | | | | |
| ACCAGCCTCAACATGTCAAGTACTCC_NODE_1_length_374_ | | ATTTTCAGCAAAAACATTTCAAGAAACTCATCTGTCATGGCT | | | | | |
| cov_48.855614 | | GTGCTGTGCAGTTCTCACCTGTTGACCTCTTGTTCTT | | | | | |
| RAD_kmer_0000429_1_TGCAGGTGTTCAGCGTAAAAACITA | 106 | GCATGAAATATTGTGAAAACTGGATTAACAAGGATCAAGTTC | A | A | W | T | GYSNP0118 |
| TATGGTTACCGTTGCTAATTGCTTTACCAACAGATTAGTCC | | TTTGTCTTAAACAAGCCAACTACATTTTTAAAATAACT[A/T]G | | | | | |
| TTATAAAGTAGTAGAGGAGAAGGGT_NODE_1_length_441_ | | ATAAATTTCTGACCGGACCACAGGCAAAATATTGGGACAACT | | | | | |
| cov_41.834469 | | GGATTACACAATGAATAACTTCTGTTGGAACTTGACT | | | | | |
| RAD_kmer_0000435_1_TGCAGGTACTCAACCTGATGATGTT | 119 | TCATGTAAATCTTGAAATTTAACCACTGTTTGTTGCATGAACA | A | C | C | M | GYSNP0119 |
| CTTGTTTTGGCAAAGAAAGGCGTTCCAAAATTGAATAGAC | | TCTTATAACACATTCAAGCAACACAAATACGTATGAA[C/A]A | | | | | |
| CATCCATTCGTGGCGATCATTTGTTC_NODE_3_length_374_ | | ACTCTCAAATTAATCCAAAACAGAATAGAGCTTTTGAGGGAA | | | | | |
| cov_15.946524 | | AATGGAAATGATGATTATAAACATTAACCTAAAAGTT | | | | | |
| RAD_kmer_0000442_1_TGCAGGTACTGTTAGTCACCCATAT | 478 | TGAAAGTGATTATATGGCAATCATCCGATAACAAAGTGTTCG | A | R | A | G | GYSNP0120 |
| ACCGGATTAGTAATTAGTATACAAAATTAGCCTATTTTAA | | AAGAATTCTGATAACTAGACCAAAAGTTGATTATTTGT[A/G]T | | | | | |
| TCATTGTTCACATCAGCAAGATGTTA_NODE_1_length_605_ | | AATCAGTTTTGGATATATGACTCTCTGTGAAACTAATTACATG | | | | | |
| cov_128.910751 | | ACCATACATAAAATAATATAACATCTTGCTGATGTG | | | | | |
| RAD_kmer_0000445_1_TGCAGGTAAACCGAAAAGCATCCG | 487 | AAATTAACCGCTAGAAACTCGTTATCATTGATCCAAAAGCTC | G | G | R | R | GYSNP0121 |
| GATATCAGGCATACCTGCATTAATCCTCGGATCCTCGCCC | | GAAACCGTTAGGTCCTCAATCGGTGTTAATATCGGTTC[A/G]A | | | | | |
| GCACATGAGATTCTCGCTACGGGTAAC_NODE_1_length_596_ | | ATTCAAAATTAAGGTCTAGAAGTTTGGCAAACTCACGCAGAC | | | | | |
| cov_148.201340 | | GGTTACCCGTAGCGAGAATCTCATGTGCGGGCGAGGA | | | | | |
| RAD_kmer_0000454_1_TGCAGGTTTAGGTGAATCAGGGAAT | 468 | AAAAAAAAAAAAACAATTAGCTGACCTTCCAAACCGGTGTG | A | G | A | A | GYSNP0122 |
| CATGTTTTCTAGTTTTGTACCTTTAAGAGGCATGAAATCA | | AAATGCACCGAGGCCCGTCCAAGATATACAACTGTTGAT[A/G] | | | | | |
| AAAATTGTTCCTTACTATATGAACGG_NODE_2_length_635_ | | GTAGAAATGTCATACACGTTAAGGTAAACAGGGGCGGTTCC | | | | | |
| cov_79.107086 | | AGGAGGGTAAAAACGCGAAGAGTCATCTCTTGGCAATAT | | | | | |
| RAD_kmer_0000456_1_TGCAGGCCCTTGCTTCAGCTCTGCC | 281 | TATCATATAAATCAGGAGGAGCCTTGGCCCTTGGGTGGCAAA | G | G | A | G | GYSNP0123 |
| ACAGTATCTGGCTGACCCCAAAAGATTGGTTTCAGTCAAC | | AACAGAACCGCCTTGAACTCAACTTCACCTTCAGCATT[A/G]C | | | | | |
| TTCAACATGGATTTGCTGAATGATTC_NODE_1_length_639_ | | AAATGACTCCATGCCAGTGGCTTCTCATCACCAAAATCCTGTT | | | | | |
| cov_90.643196 | | AAACAAAAACAAATTAGAACATATTGTGACGAACCA | | | | | |
| RAD_kmer_0000465_1_TGCAGGTCGTTCAACTGTGAAAAGT | 206 | AAGAAAACGAGCCAGTGGTGGTCAAGGCTGCTTTGAATGATG | R | G | A | R | GYSNP0124 |
| CTTGAACCATCTTCTGAAGAGAATGTAATGTTGCCGCCTT | | TAGAAAATGTTTCTGTAGCTGGTTTGGAGGGAGATGCG[A/G]C | | | | | |
| CTGAACTGCCATCAGCCGCTGCTTTC_NODE_1_length_635_ | | TCAGTCAACAGAAGTTTTGACCAAAGCTATGCCTGAGGATGC | | | | | |
| cov_160.713379 | | AGATTATGGTCTTGAAAACGGGGCGGCTGACAAGTTC | | | | | |
| RAD_kmer_0000466_1_TGCAGGACACACCACCGGGGCAGG | 395 | ACCAAATATATGTCGTTCAAAAGTCAAGTGAGTGGTGTCTAC | C | A | C | C | GYSNP0125 |
| GTGAAGGACAGCCGCACCTCTCATCACATATAACACCGCT | | TCTAACGCCACGAAATGTCCAAATGTCAACCTCTCCAT[C/A]A | | | | | |
| TCCTCTCATGTCCGCCATCCTTAATGA_NODE_1_length_544_ | | CCACAACCTTCCACGATGCTCCCTCTGCCAATCTATATATAGT | | | | | |
| cov_87.156250 | | ATTTATATCGGAGATAATAATAAAAAAAGTATAACA | | | | | |
| RAD_kmer_0000469_1_TGCAGGAACCATTTTCTATGGATTT | 215 | CATTCGTACGAATGGCAGTAAATATATATAACACAGACACGA | A | G | G | A | GYSNP0126 |
| GTAGAAATTTGTCTTAGATTTGGGTTTTTATAGGAAATTCT | | TTCGTACGAATGGGATCATACTTATATCCCATTCGTAC[G/A]A | | | | | |
| TCGCAATTATATTGGAATTTGCACA_NODE_1_length_677.cov_ | | ATGGCTATGGCCATCCATACGAACTTTACAAACTCGTCGTTA | | | | | |
| 71.431313 | | ACACGTAAAACGACATAGAAACAACGTTCTATAACCA | | | | | |
| RAD_kmer_0000470_1_TGCAGGATCTTGAGAAAGAGAGGA | 83 | GCAGCAAAAAAAAAAAAAAAAAAAGCCAAATTAACAAGAAT | A | G | G | G | GYSNP0127 |
| AAGAGAAAGCCCAAATCGTGTATGAACGAAAGAAACAGC | | GTATTGCCAAAATAATAAGTAATAACCCGTTTTCGATCC[G/A] | | | | | |
| TTAACAAACTACGAGCCAAAGCTGAGAA_NODE_1_length_ | | ATCAAAAACAAATTTTCTAACAAGAAAGTAACTTTTAAACCT | | | | | |
| 248_cov_40.653225 | | TTAGTAGGTAACGGGCGCAAGGATTTCAAGTTGGGTTC | | | | | |
| RAD_kmer_0000471_1_TGCAGGCGCTTCGTGGCAAAGAAG | 110 | TGTCAACCCCAGAATACTCGTGTTGTTCGGGTTAACGTGTCTA | A | T | A | A | GYSNP0128 |
| ATGTATCGGGTTCGTTTCTACAACTCTTTCTATTTGTTATC | | ACACAATTTTCAAGTCGGGTTAAAACCTGTCAACCCA[A/T]AC | | | | | |
| CCTTCACCGCTTAGGGGTGCTACATG_NODE_3_length_183_ | | ACGACCCATGTAGCACCCCTAAGCGGTGAAGGGATAACAAA | | | | | |
| cov_206.874313 | | TAGAAAGAGTTGTAGAAACGAACCCGATACATGTTCT | | | | | |
| RAD_kmer_0000474_1_TGCAGGTGGTGCAACTAATGATAGG | 117 | ACAACCCAAAAGAAGTTGAGGCATGACCTATTAGTCTAGTGG | C | Y | C | T | GYSNP0129 |
| CAGACGAATGCGGCTTGTGGTTCTAGCAGCAGCAGAGGTT | | TATGGTGTCCCCCCCTTACAAATATGAGGTTGTGGGTT[C/T]A | | | | | |
| CTGCTGCTCGCGGCTCAGACTCACTG_NODE_2_length_176_ | | AATCCTTGCAAATGCAAGGTAAAAATGGAATTATGGGTGTTA | | | | | |
| cov_9.312509 | | AAAAAAGTTGGCCGTTAAAAAAAAAAAAAAAAAAAAA | | | | | |
| RAD_kmer_0000476_1_TGCAGGTTACTTCAGGAATGAGCCC | 239 | ACAATAGTGACACAATGATCATTTAAAAAGAAAACTAAAAA | T | C | T | — | GYSNP0130 |
| TGGTTTTAATCTTTATGGATGCACCTTAATAGGTATGTTAA | | AAATTGGATGAAATGTGCTTCGAGCCAAACCGTGGCTCT[T/C] | | | | | |
| AGAATAGTTAAAAACAAAATACAGA_NODE_1_length_568_ | | ACTAATAAGCCATAATCTGATAATACTATCTAATACAAACCA | | | | | |
| cov_107.915489 | | CCCGACCCGTTTTTTGCCACATGTACACACCACACATA | | | | | |
| RAD_kmer_0000476_1_TGCAGGTTACTTCAGGAATGAGCCC | 331 | ATAATCTGATAATACTATCTAATACAAACCACCCGACCCGTT | W | T | T | — | GYSNP0131 |
| TGGTTTTAATCTTTATGGATGCACCTTAATAGGTATGTTAA | | TTTTGCCACATGTACACACCACACATAATCATAAGACT[T/A]A | | | | | |
| AGAATAGTTAAAAACAAAATACAGA_NODE_1_length_568_ | | CATTTGGTGAATTACGACGAGTGGTGGCAGTCACCAAAGTAA | | | | | |
| cov_107.915489 | | TACCCCCTGTATTATCATAGACTGGTGGTTTCCACTC | | | | | |
| RAD_kmer_0000476_1_TGCAGGTTACTTCAGGAATGAGCCC | 473 | AGACTGGTGGTTTCCACTCTGCAAGAACTGCGTAATGCAAGA | M | C | C | — | GYSNP0132 |
| TGGTTTTAATCTTTATGGATGCACCTTAATAGGTATGTTAA | | AAAAAGTAATATAATTACACTATCCAAATCTGTGAAAA[C/A] | | | | | |
| AGAATAGTTAAAAACAAAATACAGA_NODE_1_length_568_ | | AATTATAAATTGTGTAACTTATCTTATGGCTCTGTATTTTGTTT | | | | | |
| cov_107.915489 | | TTAACTATTCTTTAACATACCTATTAAGGTGCATCC | | | | | |
| RAD_kmer_0000482_1_TGCAGGAGGCGCTGAGAATGAAGT | 353 | ATTTTCTATCATAAATTTGTTCATTCGGAAGCCAAAAGTTGGT | G | G | T | T | GYSNP0133 |
| GTTTGGATGTTGGGTCTCGGGCAAATGACACCGGAAAAG | | ATCAAAGGACTTTTGGATAACCAATATCTAAGAGAGA[T/G]A | | | | | |
| GGCCAGGTGACCGGAAATTTATAATGCC_NODE_1_length_ | | GAAGAAGAGAAAGCTCCAAGAAAAACTTAACGACAGTTTTG | | | | | |
| 480_cov_88.708336 | | ACCTCTGGTCACACACGCGAGGCATTATAAATTTCCGG | | | | | |
| RAD_kmer_0000483_1_TGCAGGAACCAGTTCAGGGTCCTAC | 127 | CGGCTGAGGCGCACGCCTCGAGGCGAGGAGGCTAGAAAATG | A | G | R | G | GYSNP0134 |
| CCGATAGTAGGAAGAACCGCGCGTTTGTAGATCATCCTTT | | AGTCTGAGGCGGCGCCTCATCGGGTATTTTTGTACGTGC[G/A] | | | | | |
| GATCAATGTAAAGAATATCTTCTTGT_NODE_6_length_302_ | | CCTCGTAGGGATGAAGCGCTAAGAAAAGTTGCGCCTCAGGG | | | | | |
| cov_39.821194 | | GGTTTTGATGTGTGTTCTTGATGTTATAAACTTTTTGTG | | | | | |
| RAD_kmer_0000483_1_TGCAGGAACCAGTTCAGGGTCCTAC | 225 | GCTAAGAAAAGTTGCGCCTCAGGGGGTTTTGATGTGTGTTCT | G | A | G | G | GYSNP0135 |
| CCGATAGTAGGAAGAACCGCGCGTTTGTAGATCATCCTTT | | TGATGTTATAAACTTTTTGTGTCAATTTCAAACATTCC[G/A]TG | | | | | |
| GATCAATGTAAAGAATATCTTCTTGT_NODE_6_length_302_ | | TCTTTTTGATGAACCTGGTGACGAAAATAGTTGGTATTTTATT | | | | | |
| cov_39.821194 | | TATTATAAGATATATAATTTTATATTTATTTTTTA | | | | | |
| RAD_kmer_0000486_1_TGCAGGGGACTTAATGTTGACATCT | 81 | ATGCTATATATTATATGATAGTATTTTCTGGGCCCTCAGTTTA | T | T | Y | C | GYSNP0136 |
| CGGATCAGAAATACTGATTCAAAGCCATTTCTGTTTACGT | | AGGAAGGAACATGCAAGGGATTCAATTTTGGAACATA[T/C]T | | | | | |
| TTGCATATCACACCTATTTTTCTGTT_NODE_1_length_643_cov_ | | TGAAATAGGGGAAATTTTGAAAACACAATGCAAGTTTACCAA | | | | | |
| 148.662521 | | TATTTTTAAATATAGTTTACTGAATCAAAGGGGGGAT | | | | | |
| RAD_kmer_0000486_1_TGCAGGGGACTTAATGTTGACATCT | 250 | ATGCAGAGCAAAATAAAAGTGTGAAGGGTTACAATAAGCCA | G | G | R | A | GYSNP0137 |
| CGGATCAGAAATACTGATTCAAAGCCATTTCTGTTTACGT | | CAGAAACATAGAAATAAGTCTTTGGCAGATCTGATTAAC[G/A] | | | | | |
| TTGCATATCACACCTATTTTTCTGTT_NODE_1_length_643_cov_ | | GTGACTGAAATAAAGAAGCTACCATCAACAGGTCATTGCAA | | | | | |
| 148.662521 | | ATAAAATAGTCCAGAACAACGATATATCATAATAACAGC | | | | | |
| RAD_kmer_0000486_1_TGCAGGGGACTTAATGTTGACATCT | 355 | TCAACAGGTCATTGCAAATAAAATAGTCCAGAACAACGATAT | T | T | Y | C | GYSNP0138 |
| CGGATCAGAAATACTGATTCAAAGCCATTTCTGTTTACGT | | ATCATAATAACAGCATGAGCTTCTATATCTTATTTACA[T/C]G | | | | | |
| TTGCATATCACACCTATTTTTCTGTT_NODE_1_length_643_cov_ | | TTTGATGGCATTTTGTATCTATATGGTACTGACCAAGTAGTGC | | | | | |
| 148.662521 | | TTTGCATTACAATAAGAAAACACAAATTCTTATGTT | | | | | |
| RAD_kmer_0000486_1_TGCAGGGGACTTAATGTTGACATCT | 453 | ATCTATATGGTACTGACCAAGTAGTGCTTTGCATTACAATAA | G | G | K | T | GYSNP0139 |
| CGGATCAGAAATACTGATTCAAAGCCATTTCTGTTTACGT | | GAAAACACAAATTCTTATGTTGCTGGCAAGTACAAAAT[T/G]G | | | | | |
| TTGCATATCACACCTATTTTTCTGTT_NODE_1_length_643_cov_ | | CAACACAAAATGAATAGTTAATAAATCCTACTAAGTATGATA | | | | | |
| 148.662521 | | CTTAGCAGTTAGCCCCAAATAAACTAAAAACTATCCT | | | | | |
| RAD_kmer_0000490_1_TGCAGGTATTTCTTTCGATGTTTCCT | 230 | GGATATAAATATTGCTTTTCACAACCTGCCTATTATTAAAAGG | T | C | T | — | GYSNP0140 |
| GCATCCTAACCCTCTGCCAATCTGCAAATTTGCTTTCTTGT | | TACCAACAGGTTTTTTATCATTGATTTACAATTAT[T/C]GTT | | | | | |
| CTCAGAAGTGTCCATCTTCCTTTC_NODE_1_length_617_cov_ | | CAGTTTTGTGAAGATTTTAAGTTATTTTTGTTTGTTGGTGTTTC | | | | | |
| 91.534843 | | AGATTGAGAGAACTGAGCACAGCTGAGATTGGT | | | | | |
| RAD_kmer_0000503_1_TGCAGGTTAAAAATTAAGCATCCGG | 115 | CTACCTGCTAATCCACCCTTAAAAGATGCACTTGCATCTAATC | C | C | T | Y | GYSNP0141 |
| TTACCAAAAAAATATCGATAAAAACATAGGTTTCAGTGTC | | GTAATGGACCTACTTCTTTTAACATTGAGAACTCTCA[T/C]AT | | | | | |
| TTATCATTATAGCCTACAAATTGTAG_NODE_1_length_585_ | | AAGTCCTCAGATGGGTCCATCATGGTTTGATCAATACGGAAA | | | | | |
| cov_100.923080 | | GTTGAAAACTGGTCAAATGTCTTTACAAAGGGTGGA | | | | | |
| RAD_kmer_0000504_1_TGCAGGTGGTACAGAGTACAAGGA | 488 | GGTTACGTCCATTCTGGTAGGGGCCAGCTCTGCCACCACTGC | C | Y | Y | — | GYSNP0142 |
| ACAGTATGTACCTAGAAGATCCTACAACCAAAGAGGCAA | | GACCACCACGCCCATTCCCGTAACCTCTACCACCACCA[C/T]G | | | | | |
| TGGCCGTAGCAGCGGTGGTGGTGGTCGT_NODE_1_length_551_ | | ACCACCACCACCGCTGCTACGGCCATTGCCTCTTTGGTTGTAG | | | | | |
| cov_82.088928 | | GATCTTCTAGGTACATACTGTTCCTTGTACTCTGTA | | | | | |
| RAD_kmer_0000506_1_TGCAGGATCCGAAAGCTTTATTAAA | 449 | ACGAACCCTTACCCACATATATATAAGTTATATTGTTTTGCGG | T | A | T | — | GYSNP0143 |
| TCAAATTTAGTTTGACTTTTCATTATTCCCAAAGAAAGTCA | | TTCTTAAATCTGATTCAACCGGGTTGACCATTGAACT[T/A]GT | | | | | |
| GGCTAGTGTTTTCACGTTAAACCGA_NODE_1_length_535_cov_ | | AAAAAGAGCGGTTCAACGTTCGGTTTAACGTGAAAACACTAG | | | | | |
| 78.575699 | | CCTGACTTTCTTTGGGAATAATGAAAAGTCAAACTA | | | | | |
| RAD_kmer_0000517_1_TGCAGGAAGCAAAAATGGAGATAG | 341 | ACTCTAAAGTATGTATACTTGCTTATACTTTTCGCTCACCATA | C | Y | T | Y | GYSNP0144 |
| TAACAACAAAACATATCCATGAAATTAAAATGATAGTTTA | | TGCGAAATTTCATTTTCATCATATATTCCATGATTAG[T/C]TGG | | | | | |
| ATCATTCTTTTATTATGAGTAATAATA_NODE_1_length_657_ | | CACATATGCCTTTTTTTTGTTGCTCAGACTAACATGCTCGATA | | | | | |
| cov_108.126335 | | TTTCAGGTATAATGTTGCGATTAAGTGTGCCACC | | | | | |
| RAD_kmer_0000517_1_TGCAGGAAGCAAAAATGGAGATAG | 391 | TTTCATTTTCATCATATATTCCATGATTAGTTGGCACATATGC | T | T | C | T | GYSNP0145 |
| TAACAACAAAACATATCCATGAAATTAAAATGATAGTTTA | | CTTTTTTTTGTTGCTCAGACTAACATGCTCGATATTT[C/T]AGG | | | | | |
| ATCATTCTTTTATTATGAGTAATAATA_NODE_1_length_657_ | | TATAATGTTGCGATTAAGTGTGCCACCATTACTCCAGGTATAT | | | | | |
| cov_108.126335 | | TCAAGTTAATAAACCTATATTGGAAATCTAGCAC | | | | | |
| RAD_kmer_0000519_1_TGCAGGACCGCTGCCGACGACACA | 327 | GTCCAGTTAAATCTCCCATTCGGTCCGGTCCCGTTACATTAAT | A | R | A | R | GYSNP0146 |
| AACGCGCAACGTTCGGACGGTTGCAGACGAAAACGTTCG | | TAATTAACCGTTAATCCTTACAATAAACAAACAGTAA[A/G]CA | | | | | |
| AGCAAACATTGCTCTGAATATAGCCATT_NODE_1_length_470_ | | TCAGTGGCGGTTTAGAGCTATCTTCCCCTCTGCAAAACCCTAA | | | | | |
| cov_135.408508 | | ATCGAGTGTAAGTATCCTTCATCACCGATCATCAA | | | | | |
| RAD_kmer_0000520_1_TGCAGGGGTCAAACTTCCAAAAAA | 99 | TATCCACGTTGATTCTGGTGGTATCCATGTGTCTGAGGGTTTG | G | A | R | R | GYSNP0147 |
| GGTTGGTAGTATGCCTTACAAAATATACAAAAGCTGTGTT | | ACCTACTGTAATGGGTATTTGAACCATAAGGTTGACC[G/A]TG | | | | | |
| GATCTGGCGTTGACTTTGTGTTGACTT_NODE_1_length_627_ | | ACCCGGGTCATAGCTATGTGGAGGATAACCTGAACTAGGCTG | | | | | |
| cov_101.625198 | | GACCATCCTACTTGAGTCCTGACTGTAGTAGGGTCT | | | | | |
| RAD_kmer_0000528_1_TGCAGGCCCGCGGATCACAACCACA | 157 | ATCGCTCCCCACTTGTTGGAAGCCATCACCAATTCTCAAATCT | A | G | A | A | GYSNP0148 |
| CGAACATCATCAGAACCTATGTTCAACAATGAAATGACTT | | TCCTCGTCGTCTTCTCGTCCCGCTACGCTGATTCCAG[A/G]TGG | | | | | |
| CATGCACCCGAGATTCTATCCCAACA_NODE_1_length_619_ | | TGCCTTGATGAACTTTCCCATATGACCCATTGCATGTGTCACA | | | | | |
| cov_157.993546 | | CCAATTATCAACAGACCCTTCTTCCTGTTTTTTT | | | | | |
| RAD_kmer_0000529_1_TGCAGGTATATATTCTGTATAAAAA | 233 | TGCAAATATGTATACTTCTCAGACTAATCTGAATGATGCAAC | A | A | A | G | GYSNP0149 |
| GTCAACACTTTGAATTTTGGCTTTTTAAGTTTTGTTGACCC | | AGCCTGAAATGAACAAGAAAATAACAAAAAGTTAATTA[A/G] | | | | | |
| AGTTCACTATGTTTTACTGGTTTTC_NODE_1_length_576_cov_ | | GTTCAAACATTTGATAAGATCTATATGTACATTGCTTGGATAA | | | | | |
| 84.118057 | | CGGGCAAAACCGGGTTAACAAGTCATATTTTAGCGGC | | | | | |
| RAD_kmer_0000534_1_TGCAGGTTTCCGACAAAAAAGC | 317 | TGGAAAATTTCAAGAATGAAAGTCATTAGTAGGGAATCAAGC | C | T | C | T | GYSNP0150 |
| GGCAGATTCTCCGACAAAATTCCGACTAGTTTCTAGCTAC | | TAGTAAAAGAATCAAGAAGCTAAGGACTAATAATGGTC[C/T] | | | | | |
| ATTTTTTAGTGCTAGTTTTATTCCGTA_NODE_1_length_549_ | | AGAGTTATGTAGTGGTGAGTTTAACCATTTTTTTTAAACAAAA | | | | | |
| cov_80.373405 | | TGACATAACTAGATATGGTCGGAAAGAAAATAGAAAT | | | | | |
| RAD_kmer_0000536_1_TGCAGGATCACTTTCACTACATCAA | 439 | CCTCCATTGATCTGGCATTTTCCCAGATTTCAAATTTTGCTAT | G | T | G | T | GYSNP0151 |
| TCACACCATTGTTTTGTAGGTAGTAGGAGCAATGCCGAAT | | AAGATACTTAATAAGTTTCAGTTATGACATAAAAACC[G/T]TT | | | | | |
| ACAGGCAAACCAAACTCCACCAATGC_NODE_1_length_600_ | | ATATTTTTTTGCAATAACATACTTTTTAGAAGGTTAGAAATAA | | | | | |
| cov_147.115005 | | CGAGATGTAACTGATATGTTGCAACGATAGCGGAT | | | | | |
| RAD_kmer_0000537_1_TGCAGGTTCAAAGTCTTTGTGATAC | 91 | AACCCTCAATGCAAATAATTGTTGTGTTAAATATTATACCAAT | Y | T | Y | T | GYSNP0152 |
| ATTGCTCTTACCGGAACAAGAAATTGATCGGCGACCTGCT | | GATCTATGCTTTTTACAGTTGTGAACAACCCAGGCAG[C/T]CC | | | | | |
| TTTAATTGATATAGTATAGTTGTGAT_NODE_1_length_480_ | | ATTATTCCCAGACAAACTCAAATTCGCTAACCCTTTCAAAAC | | | | | |
| cov_150.695831 | | AATCAAAGTACTAACCTTTATCTCACCGCTCAACTG | | | | | |
| RAD_kmer_0000537_1_TGCAGGTTCAAAGTCTTTGTGATAC | 146 | TTACAGTTGTGAACAACCCAGGCAGCCCATTATTCCCAGACA | Y | C | Y | C | GYSNP0153 |
| ATTGCTCTTACCGGAACAAGAAATTGATCGGCGACCTGCT | | AACTCAAATTCGCTAACCCTTTCAAAACAATCAAAGTA[C/T]T | | | | | |
| TTTAATTGATATAGTATAGTTGTGAT_NODE_1_length_480_ | | AACCTTTATCTCACCGCTCAACTGTAGCAAATAAGGACACAC | | | | | |
| cov_150.695831 | | TTCTTAGTTCTTACAAAATGGTTGTTTTAGTTCGCAT | | | | | |
| RAD_kmer_0000546_1_TGCAGGTTTCATGACATAACAGAAT | 525 | AGTTGGTCAAGCACCTTCATTTCGTCATAAAGAACCTTCGTTT | A | A | M | M | GYSNP0154 |
| TTGATGATGCCAAAAGGAGTTGTCGTATGCGCTTGGCTGG | | TCCTTGAACTATCAAGAACTTTGTCCATAAGTTGTAG[A/C]GG | | | | | |
| CCATAATGAACGCAGGCGTAAAAGCT_NODE_1_length_592_ | | AGCTTTTACGCCTGCGTTCATTATGGCCAGCCAAGCGCATAC | | | | | |
| cov_167.719589 | | GACAACTCCTTTTGGCATCATCAAATTCTGTTATGT | | | | | |
| RAD_kmer_0000552_1_TGCAGGATACAATGAACCATTCCAA | 174 | AAGGTGCCAAAACATGCCTTAAATGGTTCCAATATGCTTCTT | G | A | A | A | GYSNP0155 |
| TTTGAACCTGCACTTGTAAGATTCACAGAATTATCAATAG | | ATGGGATTTCCCGATTTCGGGAGCTCTTTTTTTCTGCT[A/G]AT | | | | | |
| TAGAACTTATTGGACCCAGACCGTGA_NODE_1_length_574_ | | TCAACTTGAAGACGACTTTAGACCTCTTTTTAAGTTGCTAGAA | | | | | |
| cov_86.318817 | | ACTCAGCCAGATCCATCAGGAAAAGCCGAATCATC | | | | | |
| RAD_kmer_0000552_1_TGCAGGATACAATGAACCATTCCAA | 301 | CTCAGCCAGATCCATCAGGAAAAGCCGAATCATCTGTTACCC | R | G | G | — | GYSNP0156 |
| TTTGAACCTGCACTTGTAAGATTCACAGAATTATCAATAG | | GCATTAAAGTTGTTGACATAAATCAAATGCATTTGCTT[G/A]A | | | | | |
| TAGAACTTATTGGACCCAGACCGTGA_NODE_1_length_574_ | | AGGCGAACTCAATTTGAGCCTTCTTGATTATAATGCCCTTACA | | | | | |
| cov_86.318817 | | TCCTCTGAAAATGATCTGGGCCCTAGTCAGGCTTCT | | | | | |
| RAD_kmer_0000552_1_TGCAGGATACAATGAACCATTCCAA | 362 | AAATCAAATGCATTTGCTTGAAGGCGAACTCAATTTGAGCCT | C | T | T | — | GYSNP0157 |
| TTTGAACCTGCACTTGTAAGATTCACAGAATTATCAATAG | | TCTTGATTATAATGCCCTTACATCCTCTGAAAATGATC[T/C]G | | | | | |
| TAGAACTTATTGGACCCAGACCGTGA_NODE_1_length_574_ | | GGCCCTAGTCAGGCTTCTGATCAAGGTTTTCTTTCTGATTTGA | | | | | |
| cov_86.318817 | | CTGTTCAAGGCTCCGGTCTATCTGGTTTTTCTTCAC | | | | | |
| RAD_kmer_0000552_1_TGCAGGATACAATGAACCATTCCAA | 415 | ATGCCCTTACATCCTCTGAAAATGATCTGGGCCCTAGTCAGG | A | G | G | — | GYSNP0158 |
| TTTGAACCTGCACTTGTAAGATTCACAGAATTATCAATAG | | CTTCTGATCAAGGTTTTCTTTCTGATTTGACTGTTCAA[G/A]GC | | | | | |
| TAGAACTTATTGGACCCAGACCGTGA_NODE_1_length_574_ | | TCCGGTCTATCTGGTTTTTCTTCACTAGTGGACGAAATCTTTG | | | | | |
| cov_86.318817 | | AGCAAGAAAAGCGATCTTCTGCACCTGCGTATTCT | | | | | |
| RAD_kmer_0000557_1_TGCAGGTTCTTGCTCTGAAAGTGAG | 183 | GTGGTGGATATTCTTTCAAAATTAGCAACAGAGTTGAAAAGT | G | A | A | R | GYSNP0159 |
| GTTCTCTCTGCAAAGATATGATATAGTAAGTTAGACAATA | | CACGATTAAGTGAGGTGGTGGGTGAAGGTTTAAATCCC[A/G] | | | | | |
| ACATTACGGTAACTTTGATAAGCATC_NODE_1_length_574_ | | GTTCAACGGTCAATGTGGCTGATGAAGTCAAAGTTGACCGTT | | | | | |
| cov_87.775261 | | TAACGGATAGACCGAATACCTCAAAAAAACCAATGCCT | | | | | |
| RAD_kmer_0000557_1_TGCAGGTTCTTGCTCTGAAAGTGAG | 348 | TCTCATTTATGTCAAGTACATCACAAAAAGCGCTTCAGTCAG | — | C | C | Y | GYSNP0160 |
| GTTCTCTCTGCAAAGATATGATATAGTAAGTTAGACAATA | | CTTTGGCTTTTGCATCTGAGTTAAACTTTGGTGATAAG[C/T]CA | | | | | |
| ACATTACGGTAACTTTGATAAGCATC_NODE_1_length_574_ | | AAAGTTTGACCATTGTCTTCCATGAAGAATTTAACCATGCCA | | | | | |
| cov_87.775261 | | ATTGTGAAATGGGATCGAAGATTGCTAAACGTTTAA | | | | | |
| RAD_kmer_0000557_1_TGCAGGTTCTTGCTCTGAAAGTGAG | 483 | GGGATCGAAGATTGCTAAACGTTTAATTCCTAGGCTTGTCTG | — | W | A | W | GYSNP0161 |
| GTTCTCTCTGCAAAGATATGATATAGTAAGTTAGACAATA | | CTGACTGATTTTGGTTAATCTACAACTGAATTGTAAGT[A/T]A | | | | | |
| ACATTACGGTAACTTTGATAAGCATC_NODE_1_length_574_ | | CATCATATTTTATTTATATACAGCTGATGCTTATCAAAGTTAC | | | | | |
| cov_87.775261 | | CGTAATGTTATTGTCTAACTTACTATATCATATCTT | | | | | |
| RAD_kmer_0000562_1_TGCAGGCAAGAGCCGGTCTAGATTA | 243 | AACCCGCCAACCCGATCAACATGTACAATTTGATCACTACCT | C | G | C | C | GYSNP0162 |
| TGAGTCTTCATATGGAAAGGAACGACCCAATGATACTATT | | GACAAACGCTCTCAACACATTTACATAAATGGGTTAAA[C/G] | | | | | |
| TTGAAACTGCAAAGGACAACACCGTA_NODE_1_length_658_ | | AGGTCGTGTTGGGATTGCATGATCTTAAGTGTGTTTGGGTTAG | | | | | |
| cov_150.837387 | | AGTTGATTGTTTTTTACACATATATGCCTCATGTCTG | | | | | |
| RAD_kmer_0000562_1_TGCAGGCAAGAGCCGGTCTAGATTA | 485 | CAACTTTATAGAAAGCTTGTGTGTTGGTGAGGGAGGTGAATA | A | R | A | R | GYSNP0163 |
| TGAGTCTTCATATGGAAAGGAACGACCCAATGATACTATT | | GTGGCATACCCGTAGTAACGATCTTTGATATTCTGCTG[A/G]G | | | | | |
| TTGAAACTGCAAAGGACAACACCGTA_NODE_1_length_658_ | | ATAACTCGCCAGTGACAGGCATCTCATGCCTGTAAGGGCATT | | | | | |
| cov_150.837387 | | CAGCACCTCTAGTACATTCACCTCTGATGAAGAAACT | | | | | |
| RAD_kmer_0000564_1_TGCAGGCTGGGCTACTTAGTGCATA | 189 | TTATATTGAAAACTGATTATAGTGATCACAAGTTAGGAATAC | T | A | A | T | GYSNP0164 |
| TGTCATAAAAAGACTATATTTTGGAAGGTATATGGATTAG | | TTAGATAATCAGTTTCAAAGCGTGTATACAGAACAGAC[A/T]C | | | | | |
| GTTATGGAGGGTAACAAAAAACAAAA_NODE_1_length_593_ | | ACTGTAATAGAGGAGTAGACATACTTGGTTGTAACTCTTGAT | | | | | |
| cov_110.580101 | | TTCTCTGTGACATTATGCCAGGTGTAATGAGACATCA | | | | | |
| RAD_kmer_0000570_1_TGCAGGGAGTGAGCTTTTGACTGAT | 236 | TTTTAAGAGTACCTAAGCTATTAAGTGAAGGTGTGTGCCAGA | M | C | M | M | GYSNP0165 |
| ACCACTAAGGTGAGATGCAGCAAAAGGATTAAATTATTA | | AGTGCATACACTTCTTGTCACCCCACTGATAATAAGAA[C/A]A | | | | | |
| ATTGTTTGTCAAGTCAAATTATGCATA_NODE_1_length_589_ | | CGAGCATCTAATGATTTGTTTTGTCAGCCACTATGTCCTTACG | | | | | |
| cov_123.723259 | | AATGATCTACATAAAAGAGTTGGAATTTGCCTACTA | | | | | |
| RAD_kmer_0000576_1_TGCAGGTAATGCAAAATCTGTAAGG | 141 | CTGACTCCTGGTCAATCGAATTTTTTGGATTTTTAAACATAGA | A | T | W | T | GYSNP0166 |
| AGATGCTCATCTAATTATTGTGCTTTCAAATTGTGTATAAT | | TCTGTTTTTATTTATTTATTTATTTATTTATTTTTAC[A/T]AAAA | | | | | |
| CAACTTCTTTAGGATACAGTGATTA_NODE_2_length_268_cov_ | | CATGAGTTGGACCCCTTAATTGTAACAAGTAAAGAAAAGAAC | | | | | |
| 66.130600 | | CTTAATGTGACCCTCGACTAAGAATTAATGGTTT | | | | | |
| RAD_kmer_0000576_1_TGCAGGTAATGCAAAATCTGTAAGG | 201 | ATTTATTTATTTATTTTTACAAAAACATGAGTTGGACCCCTTA | C | C | T | C | GYSNP0167 |
| AGATGCTCATCTAATTATTGTGCTTTCAAATTGTGTATAAT | | ATTGTAACAAGTAAAGAAAAGAACCTTAATGTGACCC[T/C]C | | | | | |
| CAACTTCTTTAGGATACAGTGATTA_NODE_2_length_268_cov_ | | GACTAAGAATTAATGGTTTCGCCGCTGTACAAGTGTGTTGTA | | | | | |
| 66.130600 | | AGACTAAATGCAGCCGGGTGATCATAATCATAATCAC | | | | | |
| RAD_kmer_0000578_1_TGCAGGGAGTATATTAATCACAAGG | 453 | ATATTATATTATGTCCATGTTGATACGCGTCTGTGTGCAAAAC | A | G | A | G | GYSNP0168 |
| AAATGTTAGGAAAAAAAAACAGTTTCAACATTTCAATGTA | | TACGACTATGTCTGCAGCCTTCAGTTTAGTCATATAT[A/G]GT | | | | | |
| TTAAATATGATCCCGAAACAACAATA_NODE_1_length_598_ | | CACTTGGAGCTCATCGTTTTATTTGGCTATAAGCAAACATGAT | | | | | |
| cov_119.416389 | | CTATGTCACAATATTTTTTAAAACACTTTTTACGA | | | | | |
| RAD_kmer_0000583_1_TGCAGGCCAGGTTTGGCTTTTCAAC | 330 | ATGAAGTTTAGAATATACAAAAAAGAGATGTATGCACAATTA | W | A | W | A | GYSNP0169 |
| AACTTGCATTTTTCATTTAGAGGTGGCAATGTATAACTAA | | CCACTCCCAGTTCATCTGGATACATTCCGCGGTTCGAA[A/T]A | | | | | |
| TTACTTATGAATGGGTCGATTTGGGT_NODE_1_length_428_ | | GTGGCCTCCTTTTGCAAGTTTAGCACGGAATGTCACCTAAGG | | | | | |
| cov_46.133179 | | ATAGAAAACATCATGTTAAATTTTCCTACTAAATATA | | | | | |
| RAD_kmer_0000584_1_TGCAGGTGTTTATGAGCTTATACCT | 519 | CCGAAATAAGAATCATCAAATAAAATCAGGAACAAGCCATTT | A | T | T | T | GYSNP0170 |
| TATTATAAGGATGAGAATACAGTTTTTTGATGTTTCCCCTC | | AATAAAAAAGAAGAAGATCAACATCTTGTTAATGTACC[T/A] | | | | | |
| CCTCTGTCACTGTGAATGTTCAGCA_NODE_1_length_613_cov_ | | GAAACTTTCCAGATATTATTGCATGATCATGCTGAACATTCAC | | | | | |
| 94.779770 | | AGTGACAGAGGGAGGGGAAACATCAAAAAACTGTATT | | | | | |
| RAD_kmer_0000586_1_TGCAGGCTTCCTAAACTCAAAGGTG | 202 | GAGAGAACATACTATATCAAACCATCTATGTCTTGTGTAGCA | T | Y | Y | T | GYSNP0171 |
| ACAATTGGTGGCGAGGTCATGCAAGAGCTTTTCTGAAATA | | AGCTCGCATTCCCTACTTAAACCATCAGTCAAGAGATC[T/C]T | | | | | |
| TGTAAATTTGCCTGCCGATGCGCTTC_NODE_16_length_324_ | | CAGGAGTTTCAGTACCCCGCACACAAATCACAACAGATTTTA | | | | | |
| cov_39.268520 | | TATCATGGACTGCCACAACAAAGTATGCTGCTTCACA | | | | | |
| RAD_kmer_0000588_1_TGCAGGCTGCATGAGCAGGTTATCG | 375 | CAGCCATTGTGCACAGAACGCTTTGGTGATATTTGAGGCGGT | C | G | C | G | GYSNP0172 |
| TAATTTAATCTATTAAATGAAGAAGGTGGATTTGTAATAT | | GCATTCCTTACAGCCTAGTCATGTGAACAAAACAAAAC[C/G] | | | | | |
| CTTATTTTATGTTGGAAGAAGCAATC_NODE_2_length_439_ | | ATGTGCGCTTCGCCTATGTATCAAGGTTATAGTCTTCCAAGGT | | | | | |
| cov_71.694763 | | CTGGTGACTGGTATGATTAACAAAAAAAAAAAAAAAA | | | | | |
| RAD_kmer_0000590_1_TGCAGGTCTGTGACCAATGCCCTAA | 347 | AGGGTTGGAACCGATTTCAACATCAAATTTATAACCATTATA | R | A | G | G | GYSNP0173 |
| CGTAAAATATGAAAGGGAAGGGAACTTTATCACTGTGGA | | TGTTTGTTTAAACACTGGTAAGTTAACTTAAGCAAGAA[G/A]C | | | | | |
| TATTGAGAAAGGAATGCAAGATGGACA_NODE_1_length_432_ | | AACGTTAAAACGGCTTACTTGTCCATCTTGCATTCCTTTCTCA | | | | | |
| cov_212.203705 | | ATATCCACAGTGATAAAGTTCCCTTCCCTTTCATAT | | | | | |
| RAD_kmer_0000594_1_TGCAGGCTCCTTTTCGGTTTTCCTAT | 125 | CTACTTCCATTAAAGTTCTGCACTTTATATTGGTGTCTAGACG | A | T | W | W | GYSNP0174 |
| TTATATCAATTCTCCTATTTACATCTCTTAACGAACGGTTC | | CCAAAACCAAAAAGACCAAAAAGTTTATAACTCCTAG[T/A]A | | | | | |
| ATTGTTCCTTTAGCCCGAAACATA_NODE_1_length_588_cov_ | | TAGCAAGCAGCCAATAGAAATTCTCCAGTTTGCCCTTATTTA | | | | | |
| 80.646255 | | AATTATTCGTTAACCAACTTCCTTTTGTTGCCATGTC | | | | | |
| RAD_kmer_0000595_1_TGCAGGGATGTGTAATATGCTTACT | 310 | CTAAATCAGTCAGTATATATTTATGTTATGTACAAGTAGATGT | G | R | R | G | GYSNP0175 |
| TCTGGCTGCTGGATGGGCTGGTGCGGCTTATGTGAGGTAT | | TGAATTAGCATGTATTCGATTCTTTTAGCATGTATTC[A/G]ATC | | | | | |
| CAAGTTGATATCTATAACCTATCATA_NODE_4_length_419_ | | AAAAACAATTCAGAAATAATTTGCTATTTAGATATTATGGTG | | | | | |
| cov_31.639618 | | ATTTTTAATGATTGTTGTACTTTATTATATTGATT | | | | | |
| RAD_kmer_0000596_1_TGCAGGGAAAATTGGATCGGATGAT | 282 | TAGTTTTATTTCAAAATAAACACAAGACAGGTAAAAATACTA | W | A | T | A | GYSNP0176 |
| ACGGAGAAATTGAATTTGACCCTTACGGTTTTCGCGGATC | | ACCACCCCTCTATCAATCTATCCTACACAAAACATTAA[T/A]A | | | | | |
| GGCTTGTTTCTGATTCGGAGTTGTAT_NODE_1_length_580_ | | AAAAGCCCAAAATCGTCGCGTAAAACCAAACAAATCCTCATA | | | | | |
| cov_99.875862 | | AAACCGCGCTACATCACTAAAACTTATTCTTATAATG | | | | | |
| RAD_kmer_0000597_1_TGCAGGTTTTCTGATCTGCAATGTTT | 231 | TTGGTTGTCAGAACGCACGCTCGTTATGAAACGGGCTTCTTG | C | M | C | M | GYSNP0177 |
| GTCAATCATAACCGTGTAGCAATAAACATCACGACTTAAA | | CTTGAAACTTATAAGTCGTCTTTGTGCAGAAGATGAAA[C/A]C | | | | | |
| CCCGTTTCTTCAATTTCACTTGCAA_NODE_1_length_619_cov_ | | GACAAAGCTCTTACGCTATATAAAGCACTTGATGCATCAGAT | | | | | |
| 130.004852 | | AACAGTTCTTGTAAGATATTGTACAGTAAACTTATAT | | | | | |
| RAD_kmer_0000597_1_TGCAGGTTTTCTGATCTGCAATGTTT | 387 | TATATCTATGTATTCCCGAGTTGGAGAAATGAAGATGGCTAG | Y | C | C | T | GYSNP0178 |
| GTCAATCATAACCGTGTAGCAATAAACATCACGACTTAAA | | ATGGTTTTTTGATAAAATGATTCAAAAAGGATTAACAC[C/T]G | | | | | |
| CCCGTTTCTTCAATTTCACTTGCAA_NODE_1_length_619_cov_ | | GATGCGGTTACCTACACGATGATGATAAACGGTTATTGCAGG | | | | | |
| 130.004852 | | GTGAATCACTTGAAGGAAGCTTATTATTTATTCAATG | | | | | |
| RAD_kmer_0000600_1_TGCAGGATTTGAGCTCCCAACTTTT | 199 | GACAAAAGTTGGGTTAGTTCGAACATGTGAAAAAATTGTAAG | T | T | K | — | GYSNP0179 |
| TGGTTATAGAGCACACCACTTATCCAACAGGATATCCTCT | | AATATATTGAAGTAGAATCGGTTTGATTGAGGCCATAT[G/T]A | | | | | |
| TGCGGGAGTTATTACTTGATTTAACT_NODE_2_length_284_ | | CAAGAATGATTCACTTTAAACAAATGAAACTATTATTTGAGT | | | | | |
| cov_42.665493 | | ATATCTTCTATAAAGGATCAGACCTTTTCATAGCTAA | | | | | |
| RAD_kmer_0000601_1_TGCAGGCGACCAAGGTTAATACGCC | 133 | TTTGCATATAAACACATGAGAAGTCCTTTAAATTAAAAAAAA | K | K | G | K | GYSNP0180 |
| TCAAGTTGTGGGTATAATACCAGGTTGGATACATAAAGTA | | AGAAGTTTAAAAAAGTAAAAATATTTTCTTAATTTATT[G/T]G | | | | | |
| TGTGACTTTTTGAGCAACAAAATCAT_NODE_1_length_381_ | | TTTCATCATGTATTTATATGCAAAAGCTCTAAAATAGTTATGT | | | | | |
| cov_54.089237 | | | | | | | |
| RAD_kmer_0000602_1_TGCAGGTGGCTGTTCTGATCCATTT | 236 | AAGATCCAAAATTCAAATTACAAAATTCAAAGTTATGCAACA | C | C | Y | — | GYSNP0181 |
| ACTTATAATTGGGTTGACTGGGGTCTGGGGGTATGCTTTG | | AGAATCTAGTTAACAACGCATGTAAAACACACTACATA[C/T] | | | | | |
| ATCTCTTAACGGGTCATGTCAAACAG_NODE_1_length_575_ | | GTCTAACCGCCCCGAGCTTCTCTTCTGGTGGCATCATGGAGC | | | | | |
| cov_79.353043 | | AGTTCGATGTCGACTCCATCTGGTGGTAACTGCACATA | | | | | |
| RAD_kmer_0000604_1_TGCAGGCATCAAGATAGAAGACAT | 162 | CTGCAAAACCACCCATCACATCCCGACCACAATCTTAGTTTG | T | W | T | W | GYSNP0182 |
| AAACGCCTCTTTTCTTGAAGTTGCACAATCAACTTGGACG | | TACGGATTGTATTCATCCTTTATGTGTAGCGTTTGCAA[T/A]AC | | | | | |
| TAGTACCGGCATTTATCGCATCTTATC_NODE_1_length_590_ | | TATACAAGAGAGTATCTGCGACTATTATTGTTCCAAATGCGC | | | | | |
| cov_110.149155 | | ATTCAACATAGACGTAGTTTGTGCTACAATGTATGA | | | | | |
| RAD_kmer_0000604_1_TGCAGGCATCAAGATAGAAGACAT | 238 | GCAATACTATACAAGAGAGTATCTGCGACTATTATTGTTCCA | T | Y | T | Y | GYSNP0183 |
| AAACGCCTCTTTTCTTGAAGTTGCACAATCAACTTGGACG | | AATGCGCATTCAACATAGACGTAGTTTGTGCTACAATG[T/C]A | | | | | |
| TAGTACCGGCATTTATCGCATCTTATC_NODE_1_length_590_ | | TGAACAGAAGATGGACCACCCAAGTCACCCTCACCAGCTGAA | | | | | |
| cov_110.149155 | | ACGTTGTTTCGGTCGGATGATATCCCGTTGTAATGCT | | | | | |
| RAD_kmer_0000604_1_TGCAGGCATCAAGATAGAAGACAT | 291 | AACATAGACGTAGTTTGTGCTACAATGTATGAACAGAAGATG | T | C | C | C | GYSNP0184 |
| AAACGCCTCTTTTCTTGAAGTTGCACAATCAACTTGGACG | | GACCACCCAAGTCACCCTCACCAGCTGAAACGTTGTTT[C/T]G | | | | | |
| TAGTACCGGCATTTATCGCATCTTATC_NODE_1_length_590_ | | GTCGGATGATATCCCGTTGTAATGCTTGTAACAGTGAACATA | | | | | |
| cov_110.149155 | | GTGGGACCTTCTATCATTGTACCACTTGTTTTTACTT | | | | | |
| RAD_kmer_0000604_1_TGCAGGCATCAAGATAGAAGACAT | 363 | CGTTGTTTCGGTCGGATGATATCCCGTTGTAATGCTTGTAACA | T | Y | T | Y | GYSNP0185 |
| AAACGCCTCTTTTCTTGAAGTTGCACAATCAACTTGGACG | | GTGAACATAGTGGGACCTTCTATCATTGTACCACTTG[T/C]TTT | | | | | |
| TAGTACCGGCATTTATCGCATCTTATC_NODE_1_length_590_ | | TACTTTAGGATCCACTTAGATTGTGCTTTGCTTCCACCCAAGT | | | | | |
| cov_110.149155 | | TACTTATCGAACAGTCTACGAATGGGAGTTTCAG | | | | | |
| RAD_kmer_0000605_1_TGCAGGGAGAGTTTTACATGGTGGAT | 502 | AGAAAAGCTTCAAAATCTCCGGTGAATTCAACTTTCATATAA | W | A | T | A | GYSNP0186 |
| TTTCGTAAACTCATGGCACTTGAATCATTATCAATTAGAG | | AGATGTTTTACTTCACTTGCGAGTTGAAGCATTTTGTC[T/A]AT | | | | | |
| GTGTTCAATGGTGTTGGGATGCAATA_NODE_1_length_566_ | | TGCATCCCAACACCATTGAACACCTCTAATTGATAATGATTC | | | | | |
| cov_109.492935 | | AAGTGCCATGAGTTTACCAAAATCCACCATGTAAAC | | | | | |
| RAD_kmer_0000611_1_TGCAGGCCCACCCATATTGCCACGT | 201 | TTTCTTGTTCAATTTTGGGATACCATCCACTGTTTAGCCATTTT | Y | T | C | Y | GYSNP0187 |
| CTAGTTTTGCTTCACCCCCTCCTCTCGCTTAATTGAGCATA | | CCTTTTAATTAGTACTTTTCTTATTTATACTCATCT[C/T]GCTCA | | | | | |
| CAGAAATCTTTTTCTTTTCTCATGA_NODE_1_length_609_cov_ | | TTAGAGATATTGCATAAACCACGTCAACCCGTTCATAAATAA | | | | | |
| 185.533661 | | TGGGTCCAAAATGCGACCTTTGAAAAGCATATT | | | | | |
| RAD_kmer_0000611_1_TGCAGGCCCACCCATATTGCCACGT | 514 | TTTAGATTTCATGCAGTATACAAAAGATGTGACTTACAGCAA | M | A | C | M | GYSNP0188 |
| CTAGTTTTGCTTCACCCCCTCCTCTCGCTTAATTGAGCATA | | ACAGGGTGAGTATGTATGCACCAGCAGTTACTCCCATG[C/A]A | | | | | |
| CAGAAATCTTTTTCTTTTCTCATGA_NODE_1_length_609_cov_ | | CATAACTGCACCAAGTCTGCAAAAGCAGATCATGAGAAAAG | | | | | |
| 185.533661 | | AAAAAGATTTCTGTATGCTCAATTAAGCGAGAGGAGGG | | | | | |
| RAD_kmer_0000618_1_TGCAGGACTAGTAGTACCATTTGAC | 314 | GTATTAGCTTTTACTAATATCACGTTGCATTCTTGAACAAAGT | C | Y | T | T | GYSNP0189 |
| ATGGATCCCGAGGAGGCTGCAACTGCTCGATTCTCATGAA | | GATTTATATATGAGGATCATTAAATAAACACTTTGGT[T/C]AA | | | | | |
| TGCTTAGTTGTCTGGTGAGTTTAGAG_NODE_1_length_528_ | | CTTCCGACCCGTACTTTATAATAAATCGTTGCTTATTTATTTTC | | | | | |
| cov_162.617432 | | GTCGGTGTAGTTCCCAAAGCACTCCAAGGATTGA | | | | | |
| RAD_kmer_0000618_1_TGCAGGACTAGTAGTACCATTTGAC | 380 | ATAAACACTTTGGTTAACTTCCGACCCGTACTTTATAATAAAT | T | Y | C | C | GYSNP0190 |
| ATGGATCCCGAGGAGGCTGCCTGCTCGATTCTCATGAA | | CGTTGCTTATTTTCGTCGGTGTAGTTCCCAAAG[C/T]ACT | | | | | |
| TGCTTAGTTGTCTGGTGAGTTTAGAG_NODE_1_length_528_ | | CCAAGGATTGAAGAGCTGCTTCAGGAGGACGGTAATGTTAAA | | | | | |
| cov_162.617432 | | CGTAAAAGAGAGAAAATTCAGAAACAATCCAGCCT | | | | | |
| RAD_kmer_0000619_1_TGCAGGATACCCGACTTGCATACAA | 498 | AGAGCTAGTTCACGTGTTATAGATTAGCACGCTAACATGTTG | A | A | T | T | GYSNP0191 |
| GACATTTGCTACACTATACTTCATTGTCATATTCGATGATT | | GTGAATGGCCCATTGGTCGTTAGGCTGCATAAAGAAAA[T/A]T | | | | | |
| CTGAATGAACTTGCTATACTAGA_NODE_1_length_624_cov_ | | ATAATTTAAACAAAGAGTAATATAATAAATGTCAAGACTCAA | | | | | |
| 106.498398 | | GGCATACCTTGCATTAGATCTAGTATAGCAAGTTCAT | | | | | |
| RAD_kmer_0000620_1_TGCAGGAAACGGGTAGATCAAATG | 486 | ACTCACCGACATTCGATTTTATCACAAAAGTATACA | G | A | R | — | GYSNP0192 |
| TATCATAGGAGTTGTTTCAACCGATGAACAAATTAAAAAA | | ATAAACTGAAAAACATATATAATTGAAATAAAGTAATT[G/A] | | | | | |
| GTATGGTTAGTTGCTCAAGCCATTAGA_NODE_2_length_587_ | | AGCATTTGGAACGTGAATCGGTTCATGGTTGGAACTCAAT | | | | | |
| cov_89.657578 | | GGCTTGAGCAACTAACCATACTTTTTTAATTTGTTCAT | | | | | |
| RAD_kmer_0000622_1_TGCAGGTACAATTATGGTACTGCGG | 337 | TACTGGTATTAAATATGCTAAGGTTGTGATCAGAAGTACTAC | T | W | W | T | GYSNP0193 |
| TTTCTACTAACTGATGCTTCAGATACCTGCAGATACACTC | | ATACTCATATGCTGCTTCACAAAAAAATATAGACACCA[T/A]T | | | | | |
| AATTAGGGCAACAGCAGAAAAGGATC_NODE_1_length_605_ | | TTATCTAGAAATTTTAGCCATTTATCTTTCATGTTCATAATGG | | | | | |
| cov_137.289261 | | TTGCAGGTTCTGGGTTTGAAAGGGTAAAAAACAAAT | | | | | |
| RAD_kmer_0000622_1_TGCAGGTACAATTATGGTACTGCGG | 521 | CGTAGTTATTGTGACGTCAAATTGTATGTGATAACTAACAG | A | W | W | A | GYSNP0194 |
| TTTCTACTAACTGATGCTTCAGATACCTGCAGATACACTC | | GTTCTACCTGTAAGCTACTTATGGTTTTTATAGTTTCG[A/T]CT | | | | | |
| AATTAGGGCAACAGCAGAAAAGGATC_NODE_1_length_605_ | | TGGTGATGTGATGCATTGATCCTTTTCTGCTGTTGCCCTAATT | | | | | |
| cov_137.289261 | | GAGTGTATCTGCAGGTATCTGAAGCATCAGTTAGT | | | | | |
| RAD_kmer_0000624_1_TGCAGGTAGGTAAATTTTAGTGTGG | 403 | TCAAAACCAAAACAGAAAACAATTACCAGTTTATCAGGTGCT | C | — | T | Y | GYSNP0195 |
| GTCAAAATGGGTTGGCTCAAGTTGGCCGACCCACAATCAC | | TAATGCAATAAAGGTGGTAATTTCAACTGATGGGTCAA[T/C]C | | | | | |
| TTGTTTAATTTTTTGTATACTTATCA_NODE_1_length_650_cov_ | | GAGTTATGTTTCATCTTTAAGGGGTCAAATGGTTTAATTTAAA | | | | | |
| 82.398460 | | AGGTTTATCTGAAAACGAAACATATCAAAATGGGTC | | | | | |
| RAD_kmer_0000627_1_TGCAGGCTCACATCATCGGTCATTA | 144 | TCTACAAATTTCTCTCTCTACCCACAGTTCATGTATCACTTAA | W | T | A | T | GYSNP0196 |
| TAACCTCGGACCCCTCCACATAACTCGACTTGTGTGTAAC | | GACGGTCTCAGTTTATCGACGTGTTCAACAGCAGTCC[A/T]GA | | | | | |
| CGAAGGATTCAGCTTGGCTAAAAGAA_NODE_1_length_567_ | | TGAAACGGCGTTTTTTCAGGTGATGTTGAACCGTGAAGGAGT | | | | | |
| cov_152.495590 | | CGTGGGGTCGTTAATAATGATTCAACCGACTCTTTC | | | | | |
| RAD_kmer_0000628_1_TGCAGGTCCATAATCCATTTGGACT | 254 | AGTAAATGCAAGAAAGTTACCAATACGATTAATAAACGAAA | Y | C | Y | C | GYSNP0197 |
| CCTCTTGGCCCAGAAGCTGTTAAGGCCCACATCTGATTCA | | ACAAGTTCAAACACAATTAACTGTTTAACTAAATCAACA[C/T] | | | | | |
| AAGAAAACGTGATCCAAACACAAAAG_NODE_1_length_601_ | | GAGTAGTCAAGTTATACTAGTTCATACAGCAAAGAACAACTG | | | | | |
| cov_128.712143 | | AAAATAAGAATTATAAACTGAAACTGTTCACCCGAGTA | | | | | |
| RAD_kmer_0000631_1_TGCAGGGTGCAAACAGAATCGGTTG | 272 | ATTGCAGATTATGCAGAATGAAAAAAGAATCAGGAATCCTTT | A | M | A | C | GYSNP0198 |
| CAGAAAAGATTGCACCGATTGCTGGAAAGAAGTTTGAAA | | TTTTCATATATCTCTGGTTTATGCTCTTATAGTACATA[A/C]AA | | | | | |
| GTTTAGCAAAATGAAGATGCTTAACGA_NODE_1_length_485_ | | TGGTTTTACAATTTAAAGACGTTGTACTAATTTGTGGACAGGT | | | | | |
| cov_108.101028 | | GCTACAAAGATCTTCCAGAAGATGAATATCAGTGA | | | | | |
| RAD_kmer_0000633_1_TGCAGGCTTGTCAAATTCTTCGTAT | 425 | CAAGTTCAGATATTAAAATTATAGGCATCAGTAACACAAAGA | K | K | T | K | GYSNP0199 |
| AATTACATTCTATTCAACTGTACTGCCTGGTCCAAACTAC | | AAATTATGACGAAAAAAATAAGCGAACTTACTGATTAA[T/G] | | | | | |
| CATTGTCGCGAGGTATATTGTTCTCT_NODE_1_length_640_ | | AGTGTTTCAAAGATATTATCGGGACGAGGTAATGTTGCAAGC | | | | | |
| cov_153.359375 | | CGTGAACCCTACAATCATAAACAAAAACTTCAAACTTA | | | | | |
| RAD_kmer_0000649_1_TGCAGGTATGGGCTGTATTGAAACC | 147 | ATATCACTTAGATTGAATGTCTTGAAGACCCATACTGATTGG | R | R | A | R | GYSNP0200 |
| TGGATTCTTAGCCCTGCTAAAAGATCCATTTGATGCTGAG | | AGGAAAATGGAATTGACAAAGTTGGAGGAAAACTAAAA[A/G] | | | | | |
| CCATTGGACATAATTGTCTTTGATGT_NODE_1_length_509_ | | AAGACATGATTTAAAAAAGGAGCAAAAACAAGCAACATAA | | | | | |
| cov_204.919449 | | ACTAATTGTGGACATACAATATGTTAAGTAAATATGGCAA | | | | | |
| RAD_kmer_0000649_1_TGCAGGTATGGGCTGTATTGAAACC | 199 | AATTGACAAAGTTGGAGGAAAACTAAAAAAAGACATGATTT | G | G | A | R | GYSNP0201 |
| TGGATTCTTAGCCCTGCTAAAAGATCCATTTGATGCTGAG | | AAAAAAGGAGCAAAAACAAGCAACATAAACTAATTGTGG[A/ | | | | | |
| CCATTGGACATAATTGTCTTTGATGT_NODE_1_length_509_ | | G]CATACAATATGTTAAGTAAATATGGCAATGATACATGAAA | | | | | |
| cov._204.919449 | | AAGAAAGCCATATCTTAGTAAAGCACGTCTCCTAGGAAGT | | | | | |
| RAD_kmer_0000651_1_TGCAGGGGGCGCCTGGCAAC | 202 | GACATATTTTGGAGCCGAAACTTCTATAGGCTTTTTGATTTGC | Y | T | T | C | GYSNP0202 |
| ACATTGATGCAACTTTGGGTAGTGGAAACCTGAGAGAAG | | ACACCATCAGCCCATCGGTACTCATACCTGTAAATTT[T/C]AT | | | | | |
| CTGTGAGACTTCCTCCTGGGGAGGATAT_NODE_1_length_588_ | | TTAAATAAAAATTTAAGGATCATGTATGTTTATATATAAGAT | | | | | |
| cov_137.096939 | | AGATTAATTTTAATAAACAAAAAGAATCAGTATAAC | | | | | |
| RAD_kmer_0000653_1_TGCAGGATTGTAAATGAAACCAC | 394 | ATGATTGGTGTTTTACTTCTTAACAATTCATATTTCAATTATG | Y | T | Y | T | GYSNP0203 |
| AATTCAATTAATGCAAAGTATCTTATGATCAAAACAAATG | | TGGGTTGCAGTACATCTATATGTAATATGATGGCAAA[T/C]TG | | | | | |
| TTCATATGAATTTCATTAGTTACAAT_NODE_1_length_457_ | | TAACTAATGAAATTCATATGAACATTTGTTTTGATCATAAGAT | | | | | |
| cov_100.118164 | | ACTTTGCATTAATTGAATTGTGTTGTTTCATTTAC | | | | | |
| RAD_kmer_0000654_1_TGCAGGGATACCAAATTTGGGTGTT | 153 | ATTGCTGGATTGATAGGTGGCGTTTTTAGCTTAGCTTTGTTGA | R | R | G | R | GYSNP0204 |
| TAATGTCACCAAGTTTCGTTACTTCTCTTGCAAATTCTTTC | | CATTCATCATGTGGTATAGAACTCAACAATACCAAGA[G/A]A | | | | | |
| TTCCCTTTGACAATCCCTTCCTTCA_NODE_1_length_279_cov_ | | GGAACCACTCAAAACAAAGTGCTGAAAAACAAGGGAATCAA | | | | | |
| 12.491039 | | CAAGAAATATCTTCGGTTCTTGCCACATCAGCACCTAA | | | | | |
| RAD_kmer_0000655_1_TGCAGGGGTACTTAAATTAGCCA | 145 | TGCCTTAGTGAAGAGGGGTGCCCTTTCTAATTCTTGAAAAGG | G | G | G | A | GYSNP0205 |
| AATATAGGGAAAAAAACGCATGCATTATCATGCTGTACA | | AAAACTGTATAGCTTCCCTATGGAGACTAAGATCTAAT[G/A]A | | | | | |
| ATGGTACATTTTAGTCATTGTACCTTT_NODE_1_length_569_ | | CTCCCTAAGACTTCACTTAACCAAGATATTTTGTCAGCTTCCT | | | | | |
| cov_133.080841 | | CAAAAGCAGTTTCCCAGAGAGTATTCAGTATATCCC | | | | | |
| RAD_kmer_0000659_1_TGCAGGATTGGAAGGTCAAAACGCT | 318 | AACAAATTCATTAACGTGGTAATCTTTATAGACCCAAACAAT | A | A | A | C | GYSNP0206 |
| CCGGTAAAGCATTAATCATTTGGCTATGGATTGTCGTTTA | | TGAAAATGACATTCATTTTGAGAAACAGAAATGTAACA[A/C] | | | | | |
| TTTTGTGTGTCGGTTATTATCTAGTT_NODE_1_length_433_cov_ | | TAAATGTTCATTTTTTTTTACTGAATACCAGTTTTGAGCATCA | | | | | |
| 94.254044 | | ACAGAAAAACTAGATAATAACCGACACACAAAATAAA | | | | | |
| RAD_kmer_0000670_1_TGCAGGCTAAAACAATAAATCAGCTCT | 176 | TGTGATCTTTTCGGAAAAGCGACTTTTTTGCTGGTCTTTTAGT | T | A | A | T | GYSNP0207 |
| TTGATTCGGCCCTATATACTTATATCAGACAAAGCATCA | | TGAATTACTAAGTTTTTTTTTTAGTTTTTAATTTTTA[A/T]TTTT | | | | | |
| CATACAACATCTACGAAAACCCTGCG_NODE_2_length_252_ | | TTTTTAAATGTGTGATTTAATTTTGATACAGATAAGTCAAAGG | | | | | |
| cov_38.940475 | | GTGTTTGAAGTTATGAGGAAGAAAGGGTTAGAT | | | | | |
| RAD_kmer_0000679_1_TGCAGGTCTTTGATTTTAAACCGAT | 88 | TGAAATCTGAATTTGATGAATATGAACTCTAGGTATTCTTTTG | R | R | R | R | GYSNP0208 |
| ACAACCAACACAAAGGGTTCAAATCAGCTGCAAACCCAC | | GGGTTATTTCTTTTGTTATATAAGCTCGATGATGACG[A/G]GC | | | | | |
| CATCATACACCCTTAGCCAACAACATA_NODE_1_length_196_ | | CAATGCTCAGTTGAGAGCTTCTTGGATGTGTGGTTTCATGCTA | | | | | |
| cov 262.433685 | | TGTTGTTGGCTAAGGGTGTATGATGGTGGGTTTGC | | | | | |
| RAD_kmer_0000684_1_TGCAGGCTGCAACTTATGTCTTTAC | 407 | CAAGTTGCTTATACTCAAAGCAATCTAGTTTTGTGTAATTGTT | Y | C | C | Y | GYSNP0209 |
| TCTATACATAGCCCAATAAGAAGTGTTTATATTGTGCCTA | | ACTTACGTAACTGGAACAGGCAGTTGGTGTATAATAG[C/T]TA | | | | | |
| ATACTCTCCAATAATTACTGATCATT_NODE_1_length_578_ | | AGAATAGAGGAAACCTCGGGTTAATTTATACCGGTAATTCTT | | | | | |
| cov_139.062286 | | TTAAATATATGTGTTGCAGTCTGGTAAATGGTTTTG | | | | | |
| RAD_kmer_0000687_1_TGCAGGGTAAATGCAGTTTTCTTTTT | 293 | CCCTAGCATTTAACAAATATTATCTCTATTTTAGCCTTGGTTG | R | G | G | R | GYSNP0210 |
| GGAGTAGAAACATGATTGATTTTCATGTCATTTCGACGTT | | ATAGACCCTTCAAAAATTAAAATAGAAGTTTGACTTT[G/A]AA | | | | | |
| CACATTGGTATCTTAACAATCTATT_NODE_1_length_652_cov_ | | AGTCATCCTTTTCCTGATGTGTCAAAATGTGTGGTTTGGGTAA | | | | | |
| 68.575157 | | TGGGTCAAAGACGTAACATTTCGTATGGACTAGCC | | | | | |
| RAD_kmer_0000687_1_TGCAGGGTAAATGCAGTTTTCTTTTT | 372 | TGAAAGTCATCCTTTTCCTGATGTGTCAAAATGTGTGGTTTGG | S | G | C | G | GYSNP0211 |
| GGAGTAGAAACATTGATTGATTTTCATGTCATTTCGACGTT | | GTAATGGGTCAAAGACGTAACATTTCGTATGGACTAG[C/G]C | | | | | |
| CACATTGCTATCTTAACAATCTATT_NODE_1_length_652_cov_ | | AAGTTTGGTTGACCCCTAACAAAATTCTATTATCCTGATTCAA | | | | | |
| 68.575157 | | ATTCTGTTTTTTTTTTTTTTTCCTGAATAATATGAT | | | | | |
| RAD_kmer_0000688_1_TGCAGGCGAGATGACGATGACGATT | 217 | TTTTAATCTTTTCCAAAGATTTACTTAAAAAATGAAAACATGA | G | — | G | A | GYSNP0212 |
| GTGAAGAAGATGACGATGATCTGGCAGGGGTTGAGATTG | | GAAAGTCGGTGATAGGCCATTGGGCTTGACCCACTTT[G/A]GT | | | | | |
| CATGGAAGCATGAGGCAGCTGCCATTG_NODE_1_length_597_ | | TAAACCACTTTGATCTGTAAAGAAACGGGTTGACCCGTAGAA | | | | | |
| cov_103.852600 | | CCCACCCGGAAAACCCAAGTACTTGCTTTTGTAAAA | | | | | |
| RAD_kmer_0000691_1_TGCAGGCTGCTGCTATCGTTTATTTT | 197 | TGTTATAGTCAAACATCACTACAACACACCACTCGGTGCCCA | W | A | W | W | GYSNP0213 |
| TCAACCTTGGCCGCTAGTCACCAACCCATCTATGATTTAA | | AAAGCCCTTGAGCGGGAATCATTTGACACCACCACACA[T/A]T | | | | | |
| TTCTTTCTTCTATTTCGTTTTTGTT_NODE_1_length_617_cov_ | | TTTCATTTTTATTTCTTTTATTTAATTTTTACTTAAAAAAACTA | | | | | |
| 128.870346 | | CACCCATGTGAAATGATGATTTAGTTAAAGGTTCC | | | | | |
| RAD_kmer_0000691_1_TGCAGGCTGCTGCTATCGTTTATTTTT | 340 | GATTTAGTTAAAGGTTCCCTGGCTACACGGCAATGACGTAGC | W | T | W | W | GYSNP0214 |
| TCAACCTTGGCCGCTAGTCACCAACCCATCTATGATTTAA | | ACTTTAACAATAACGTGTGGCTAAACCACTACATTTAG[A/T]C | | | | | |
| TTCTTTCnCTATTTCGTTTTTGTT_NODE_1_length_617_cov_ | | TAATCTCTTACTAAATGCATTAATATAATTATAATATAATGTG | | | | | |
| 128.870346 | | TTTTACATTATATGTTTTTTTCTACACATTGTAATT | | | | | |
| RAD_kmer_0000693_1_TGCAGGTTTTTGTACAGCACCCATT | 354 | ATGTTGGGCGAGCTTAAAAAGCTGATTGAAGCGAATCCTCTT | A | M | M | A | GYSNP0215 |
| AATGGGTTATTTACAGGGGACGGAGCCCTGGCACCATTTG | | TTTCGTGATAAGCTAAACTTCCCAGCTTTGAAAAATTC[C/A]A | | | | | |
| GCTGTGACTGTCCACAAGAATGGTCA_NODE_1_length_601_ | | GATTGCGTACACTTATCAATCAAAGGTTCATATTAGATACTCC | | | | | |
| cov_148.986694 | | CCGTATCTGGGTCTGACATTTCTTGTTCAAGTTCAC | | | | | |
| RAD_kmer_0000695_1_TGCAGGTGCAATCAAGATACATGTC | 232 | CATTTTTATCCCGTTAAAAAGCTTAAATGCTTTTCAAGACATT | G | G | K | — | GYSNP0216 |
| CCTAAATGAATGGTTTTCTCTGAAATAAGTGGTTCACTAC | | GGCATACTAACTTAGTATACTTTAGGGTAGTTTATCA[G/T]AA | | | | | |
| CTTTAAGGTAATCACAGACCATGATG_NODE_1_length_552_ | | ATTCGTATTTATCATGTTAGAAAGCTTATATGTTTTTTTTCAA | | | | | |
| cov_176.846008 | | AATTCCATTTATATAACTCCAGTTATGTGCTGCCT | | | | | |
| RAD_kmer_0000697_1_TGCAGGTGTGAAAGTTTCACTGGAA | 253 | ACCTACCTAAATGAGGGGCGTTTTGGACTTTTTGCTTATGTTT | A | T | A | T | GYSNP0217 |
| AAGAATAACATTTGGGCTTTGGTTGTTTTGATTGTAGGAG | | ATATCAATAAATACTCTTTATTTATGTTGTTCCCCTT[A/T]TAA | | | | | |
| GGTTGGTTGTTTTTTGTTGCTTGATA_NODE_1_length_560_cov_ | | ACCAAAGTAAACCCTAAAATCACATGGGTTGAACCGGTTATC | | | | | |
| 107.194641 | | AAACTTCTATTAAAATTATTCGTTGCAGCCTGTAA | | | | | |
| RAD_kmer_0000702_1_TGCAGGTCCCTTTGGTTGCAGGCAC | 229 | CGAAAAAAAAAGTGATCACAAAATTGCTTGAGATAGACAAA | T | T | T | C | GYSNP0218 |
| AGTTAACCGTGGTAGTGAAGTGATTGCCGGTGGGTTAACC | | CAGAATTCGGTAGCAGGGGCAAGAAGGACATAGACAACA[T/ | | | | | |
| GTTAATGACTGGACTGCGTTTTGCGG_NODE_1_length_505_ | | C]GAAAACTGATCACAAAAGTTGAACATATAGAGACAGCCTA | | | | | |
| cov_182.188126 | | ACTTTCAACAACATACAAAGTTAAAAAAAAGGAACATAAG | | | | | |
| RAD_kmer_0000714_1_TGCAGGCACAACCTCACTACCATTT | 530 | AAGCAAAACAAGTAGAACAAACAAAAAAAAAGTTTATTGTT | T | T | T | C | GYSNP0219 |
| CATTTTGACAACTTTAAACTTTTTTATTTGTCACACTTTAA | | AAGTGGAAATTGATTCATACTGATTGCTAAAATAATGTT[T/C] | | | | | |
| ACTTTTTTATCGTTACGTCTTAAAA_NODE_1_length_628_cov_ | | CTACAAAGTAAAGAATGTTGGATGATCCATTTGTTTTAAGAC | | | | | |
| 63.117836 | | GTAACGATAAAAAAGTTTAAAGTGTGACAAATAAAAAA | | | | | |
| RAD_kmer_0000722_1_TGCAGGGAGACTTGTGTGCCG | 201 | GAATATGAACATATGATAACGCTGCTTCATTTATGTTTGTGAA | C | M | M | C | GYSNP0220 |
| AGCAAATACAAACGAGCCATGTTCATGTTCGTTCATTTTA | | CGTTCATTTGGTTCATTTGAATTTGTTTGTGTCAGTT[C/A]GTT | | | | | |
| CTTTAACTAAACATATGATCGAACAT_NODE_2_length_517_ | | CGTTTACAGTAATATCCGTTAGTTTACGTTCATTTATTCGTTT | | | | | |
| cov_61.665379 | | ATATTTCTAGATTCATTACAATTTAGTCTAAATT | | | | | |
| RAD_kmer_0000726_1_TGCAGGCATATAAACAACAAGACG | 353 | AGGATTGGAAGCCTAAGAAAATTCACTCGGATCACAAACAAT | A | M | A | — | GYSNP0221 |
| CTGAAATTACGAACCACCACTAACAATAACATCAAATTAA | | TGTTCACACGTACAATACTAATTGTGATGAATTACGTT[A/C]G | | | | | |
| TAGGAAGCAGAAGGAGATGAATCGAAG_NODE_1_length_576_ | | AGAGAGTATAAATTACAACAAAAATGTATCGTTTAGTGTAAA | | | | | |
| cov_78.984375 | | GAAACTGAATTAAAGAGTACCTGAAGAAGAAGAA | | | | | |
| RAD_kmer_0000730_1_TGCAGGCTCATAAACTATTTTCTTTT | 131 | CAAAATCCATAATCCCAAAACCATTCTACGTATCTGAACCGT | C | C | C | T | GYSNP0222 |
| TGTTAGTTCTGCACTTAAGTATGTCAATGACTCAATGGTG | | TTCTTTAAAATGACCCGCGACGGGAATACGGTTGTTAC[C/T]G | | | | | |
| TCAGACGTTAGGACTCTTTCAATA_NODE_1_length_546_cov_ | | TCTTGCTTATTTGTGGTACCAAAATTCGCTACAGCAACCGGTA | | | | | |
| 117.739929 | | AAAAAAACCTAAGTTAAAGAGTATTTTTATCAAAAG | | | | | |
| RAD_kmer_0000736_1_TGCAGGTGAACTACTCTGCCTGAGC | 494 | TCCTCTGGTAACACTGGTAGTGGTTCAAGTTCTAATTAATA | G | K | G | K | GYSNP0223 |
| AAATTATTGGGCAGATCCATAGATGAAGAAACGGGTCGG | | ATATTATTAATCAAGTTGAGAGCTTTGAAAAACAGTT[G/T]AT | | | | | |
| GTATTTTGACCCGGTTGACCCTGAAAC_NODE_1_length_624_ | | GATGGATAATATAAAGCAAGAACAAGATGGGGCTTATGTTTA | | | | | |
| cov_115.104164 | | TCAGAATCAAAGTCAAAGTCAGTTTCAGGGTCAACC | | | | | |
| RAD_kmer_0000737_1_TGCAGGTAAAACCGTAATAACCAGT | 270 | CGTACGTACATTACTCTAATGTTGAATTAAACATAACAAAAT | T | C | Y | Y | GYSNP0224 |
| GATCATGCACTAAAGTTAGAGACAGTGCCTGAGTGGATA | | ATTATGACAAGCATCCTATAACCTTAAGCTTGACAACC[T/C]G | | | | | |
| GTTATTGTTGGAAGCGGTTACATTGGT_NODE_1_length_547_ | | GATTATATCATTTACATGGAAAAACACAAACACATCAAGCAA | | | | | |
| cov_173.469833 | | CAAATAAACCGGTTCAACGTATATGAGGTAAAAACAA | | | | | |
| RAD_kmer_0000739_1_TGCAGGCTGCACTAAAAAGCAATCA | 172 | ACATAACAATATACTAATTAAATTTTATATATATTACATTAAT | S | S | G | S | GYSNP0225 |
| AGCTTAAAAATTTATGATCTAAGACAGTGTTGACATGATT | | TAAGTATGATGTCATCTGGTTCAATGGTACGACCGAT[G/C]AC | | | | | |
| ACCTGGACAATCATCGCGTGGAGTTC_NODE_1_length_567_ | | CGATCTGACCATTAAACCAGTAGTGTGGTCCGATTAGTCCAC | | | | | |
| cov_136.061722 | | GTTTGATTTTATAAGTTAAACGAGTTATTTTTACAT | | | | | |
| RAD_kmer_0000739_1_TGCAGGCTGCACTAAAAAGCAATCA | 268 | AAACCAGTAGTGTGGTCCGATTAGTCCACGTTTGATTTTATAA | K | K | G | K | GYSNP0226 |
| AGCTTAAAAATTTATGATCTAAGACAGTGTTGACATGATT | | GTTAAACGAGTTATTTTTACATTGGATATGATAAGAG[G/T]TT | | | | | |
| ACCTGGACAATCATCGCGTGGAGTTC_NODE_1_length_567_ | | TGAAGTTGAGAAAATACTAGATGATGTTAAACTATTAATGGT | | | | | |
| cov_136.061722 | | AATCCGAGTTTTGACTATTATCGATCGATTAATGCA | | | | | |
| RAD_kmer_0000740_1_TGCAGGCTGAGACGGTCTGCCACTC | 323 | ATGTTCATTGGGGCATTTCGTCGAACAGGTTAAGGGCATATG | Y | C | Y | C | GYSNP0227 |
| CGCCACCTGGTTCGATTCCAGCTTTCTTGCACCGTTTATAT | | TTTGCCCTTAAACAAACTACTTGTAATGAATTTTATCT[C/T]CA | | | | | |
| TGTATCTGGGTGTTCTAATCCTAAT_NODE_1_length_609_cov_ | | TTGATGAACATGGGATTCATTATATTTGTTCTTACTGAATTAT | | | | | |
| 209.612473 | | TATCTCGTTTTATTGAATAAAAATTGGTGGGTTAG | | | | | |
| RAD_kmer_0000746_1_TGCAGGTTAACCAAACTGAATATGG | 96 | ACTCACAGTTAGCCAATGAACCATTCATATTTTACAAACATTT | — | G | C | G | GYSNP0228 |
| TCTAACGTTTTCTTGAATCATAATATAAACCAAAGTACGT | | CTAGCTTTTATATCTATAAAGCAAAAAAATAACGCAG[C/G]TT | | | | | |
| AATAGGTTAAAAAACAATTTGATTAT_NODE_1_length_575_ | | TGGTTTTAGTTATTAACAGATTCGAACTGAAAATCATCAATAT | | | | | |
| cov_73.747826 | | GACCGAATAAACCGAAAACTGATTTGTTAATAACA | | | | | |
| RAD_kmer_0000746_1_TGCAGGTTAACCAAACTGAATATGG | 278 | TTTTTGTTGAGGTTAATAACCGAACAAAACCGAACCATGCAT | — | A | G | A | GYSNP0229 |
| TCTAACGTTTTCTTGAATCATAATATAAACCAAAGTACGT | | ACCCTTGTCAGTGGCTATGTTTCCCGTTGCATCGCACG[G/A]A | | | | | |
| AATAGGTTAAAAAACAATTTGATTAT_NODE_1_length_575_ | | CACTCTTCTAGTATAGAAGAACCTATAATCGGTTGTATTTTAT | | | | | |
| cov_73.747826 | | TTGCATGGGTTATAAGATTATCCCTCATCAATATCG | | | | | |
| RAD_kmer_0000746_1_TGCAGGTTAACCAAACTGAATATGG | 334 | CTATGTTTCCCGTTGCATCGCACGGACACTCTTCTAGTATAGA | — | T | A | T | GYSNP0230 |
| TCTAACGTTTTCTTGAATCATAATATAAACCAAAGTACGT | | AGAACCTATAATCGGTTGTATTTTATTTGCATGGGTT[A/T]TA | | | | | |
| AATAGGTTAAAAAACAATTTGATTAT_NODE_1_length_575_ | | AGATTATCCCTCATCAATATCGTATTTCTCGGAAGACTTAATC | | | | | |
| cov_73.747826 | | CAGAAACCTTGACCAGCCTACACTAAATTTTTGTG | | | | | |
| RAD_kmer_0000746_1_TGCAGGTTAACCAAACTGAATATGG | 445 | TCGGAAGACTTAATCCAGAAACCTTGACCAGCCTACACTAAA | — | T | A | T | GYSNP0231 |
| TCTAACGTTTTCTTGAATCATAATATAAACCAAAGTACGT | | TTTTTGTGAGCCAATGATAATCATGCTTGTAGAAAAAT[A/T]A | | | | | |
| AATAGGTTAAAAAACAATTTGATTAT_NODE_1_length_575_ | | AATTGAATTTTATCAAATATAAGAAACCTAATTACTTATAGC | | | | | |
| cov_73.747826 | | AAGGTATTGATGTAAACTACAATAATCAAATTGTTT | | | | | |
| RAD_kmer_0000748_1_TGCAGGGGCGTATGCTATGCATGGT | 112 | TTATATTCTATTTAACCAAATGAGGTTTTCACACAATAAAATG | R | A | G | G | GYSNP0232 |
| TTTCGCATGAATTTGGTTGAGTTATGAACGATTTAGACTTT | | CTTCTATGTGGAGTAATTGAAATCTGTAAAAAGCAAA[G/A]GT | | | | | |
| AGATACACTTACATAGATTAATTAT_NODE_1_length_618_cov_ | | ACCAGGCATCATCTCCCTAATAAACAAAGGTATTGATGAGAT | | | | | |
| 152.051773 | | GGAAGCGACCATCTTGGGAGACTGATTACATTTGAT | | | | | |
| RAD_kmer_0000748_1_TGCAGGGGCGTATGCTATGCATGGT | 513 | AGTTTATAACAAAGATCAACAAAAAGAGGTGTTTGATTCATC | R | G | A | A | GYSNP0233 |
| TTTCGCATGAATTTGGTTGAGTTATGAACGATTTAGACTTT | | TCTTGCCATCCTGATCACTTCAACCTTCGGTGCCAGTG[A/G]TT | | | | | |
| AGATACACTTACATAGATTAATTAT_NODE_1_length_618_cov_ | | TTACCTGATAACCATATAAAACAAACTTATATTAACTCATAA | | | | | |
| 152.051773 | | TTAATCTATGTAAGTGTATCTAAAGTCTAAATCGTT | | | | | |
| RAD_kmer_0000748_1_TGCAGGGGCGTATGCTATGCATGGT | 563 | TCCTGATCACTTCAACCTTCGGTGCCAGTGATTTTACCTGATA | Y | T | C | C | GYSNP0234 |
| TTTCGCATGAATTTGGTTGAGTTATGAACGATTTAGACTTT | | ACCATATAAAACAAACTTATATTAACTCATAATTAAT[C/T]TA | | | | | |
| AGATACACTTACATAGATTAATTAT_NODE_1_length_618_cov_ | | TGTAAGTGTATCTAAAGTCTAAATCGTTCATAACTCAACCAA | | | | | |
| 152.051773 | | ATTCATGCGAAAACCATGCATAGCATACGCCCCTGC | | | | | |
| RAD_kmer_0000749_1_TGCAGGTAATAAATGAGAAATGGA | 223 | CTTGGCTTACTCTTAAAATTGTTGAATGGTGAGCTGGCATGGT | G | T | G | — | GYSNP0235 |
| AAACAAGTGGCTATGACACCGTATTTATTTAATTTTTCTGT | | CTACACGTCGGGAAGTTCTAAAGGTATGTTATTAAGT[G/T]CT | | | | | |
| ATAAATCTTACAGTTGAAAAATAATC_NODE_1_length_513_ | | TTCTCTGGCCTTTTGGGTAAATAGGTTTATTCTAAGACTTTCT | | | | | |
| cov_139.072128 | | GGAAGTGATGAATAGGTTCTTGGCATAATGGGTGC | | | | | |
| RAD_kmer_0000749_1_TGCAGGTAATAAATGAGAAATGGA | 437 | GTGATGGTACTCGCGCGGCCAGTGATGCGGGCCCACACATTC | G | G | K | — | GYSNP0236 |
| AAACAAGTGGCTATGACACCGTATTTATTTAATTTTTCTGT | | AGTCCAGCGATGAGTTGATGGATCTTTGGCCATCATTT[G/T]C | | | | | |
| ATAAATCTTACAGTTGAAAAATAATC_NODE_1_length_513_ | | GACATCGGAAGATTATTTTTCAACTGTAAGATTTATACAGAA | | | | | |
| cov_139.072128 | | AAATTAAATAAATACGGTGTCATAGCCACTTGTTTTC | | | | | |
| RAD_kmer_0000750_1_TGCAGGCTTTGTATATCTGGAACTT | 192 | TGTAATACCTGATTCAACCTGGTACTTTACCCGTCCAGTTGAC | T | T | C | Y | GYSNP0237 |
| GTTATGTGAAGGAGCATATGTATATGCATGTGGTGATGCT | | AAAAATATTAACGTCGTAATGTCAGCACTACATCAAA[C/T]CA | | | | | |
| AAAGGCATAGCCATAGATGTACATCG_NODE_1_length_623_ | | TATTGGCAACAAAAATCATCTTGCTTCAATAAATATAAAAAT | | | | | |
| cov_100.709473 | | ATTAATGAATGCCATAACACGACTTTCAAACAGAAG | | | | | |
| RAD_kmer_0000752_1_TGCAGGCAAGAAGGACAAAACTCA | 433 | AAGTTATTATGTGTGAAAAAAGTCAGCTTATCATTAATTCTAT | G | A | R | G | GYSNP0238 |
| AAGTGAAATTGTGGCAAACTTGCATAATAATAGTTGATAA | | TAAAATTGTGGCACTAATTTGAACTCCTCTCAAGTGA[A/G]AC | | | | | |
| TACAAACCAGACGGTGCAAAGACAAAT_NODE_1_length_632_ | | TCACATGTCAGACCCCACTATGAATGTTTTTCAATGTGATCTG | | | | | |
| cov_83.754745 | | AAGCCTTATTGTGTATTACAGTGTCTAAAAATGTT | | | | | |
| RAD_kmer_0000759_1_TGCAGGCAGCCCCATTCCACTACTA | 235 | CATTATATATAGTATAGATATAGGATTGTAATATATAATTTTG | — | G | G | T | GYSNP0239 |
| TGGAGTGCATCTCCTCCGTCTTTCGTTGCGATGATACCCGT | | CATTATGTTATATATTTTAAGGCAACATAATACAAGT[G/T]AA | | | | | |
| CACTTTGATGGCCGTTACCTTGGCT_NODE_1_length_601_cov_ | | ATATAAGTAAGAAAAGAAAAATAAAAAGAGTATATATTTTTA | | | | | |
| 108.088188 | | ACTCCTCTTCGTAGTATATATGAAATTAAAAAGCTA | | | | | |
| RAD_kmer_0000765_1_TGCAGGTTTACTATAAAGCAACTAA | 307 | AACGAAGAAGTGGAATCATTTGCTAGATGTTTAAGGGTGTGT | A | G | A | R | GYSNP0240 |
| CTTGCCCACATCTTCCTGCGTCGTCTTTTTGGTGGAAGCGG | | AAATTAGTTGGTTTAGATATTAATCAAGATTACTTTCC[A/G]C | | | | | |
| CACCCGTAGGGCCGAAGGATCCACG_NODE_2_length_457_ | | ATCGGGTTTGCAGGCAATTTGGGTATGCCCAAGATATTCCAG | | | | | |
| cov_68.757111 | | CAGATATCATACTTGTTGACGACAATGAAGATGCTTG | | | | | |
| RAD_kmer_0000765_1_TGCAGGTTTACTATAAAGCAACTAA | 358 | GGTTTAGATATTAATCAAGATTACTTTCCACATCGGGTTTGCA | C | T | C | T | GYSNP0241 |
| CTTGCCCACATCTTCGTGCGTCGTCTTTTTGGTGGAAGCGG | | GGCAATTTGGGTATGCCCAAGATATTCCAGCAGATAT[C/T]AT | | | | | |
| GACCCGTAGGGCCGAAGGATCCACG_NODE_2_length_457_ | | ACTTGTTGACGACAATGAAGATGCTTGGGTTGACTATTCTAC | | | | | |
| cov_68.757111 | | ACCGCTGTTGAATCTAGAGGTATACATTCCACCAAT | | | | | |
| RAD_kmer_0000768_1_TGCAGGATGACAAAAGACGTTATTC | 182 | TATAGCAATGATATGCACTTTTACGTTCGCTATCAGTTCATAA | — | G | G | K | GYSNP0242 |
| AAATTACAAAATTAAATTCAGGAAAAAATAATTAACACC | | TCTGGATCCTTTTCCTCTACAGCTGTCAAGCAAAACT[G/T]TA | | | | | |
| GAAGAACTACCTTAATTGACAACCTTA_NODE_1_length_546_ | | AAAGGCAAGAAGGGTATGGTGTATTAACAGTTCTCATATTTC | | | | | |
| cov_81.430405 | | TGCTTCTATCTTTCTACCTGTGGCTTGACATGGGGC | | | | | |
| RAD_kmer_0000768_1_TGCAGGATGACAAAAGACGTTATTC | 338 | GGGGCACGGGGAGATGATTACACTAAAATGTGTTTTCTTTAA | C | Y | C | T | GYSNP0243 |
| AAATTACAAAATTAAATTCAGGAAAAAATAATTAACACC | | ATCTTTCTAATCAGTAATTGGAGAACTACCAGCAAATA[T/C]G | | | | | |
| GAAGAACTACCTTAATTGACAACCTTA_NODE_1_length_546_ | | CAGTCAGATGAACTGAATGATTCTGAAAAGTTGACCGAAAAA | | | | | |
| 6_cov_81.430405 | | GGATATAAGACTGTAAGCCAGCAGGAGTATATAAAAC | | | | | |
| RAD_kmer_0000771_1_TGCAGGCTTTGGTACAGAGCCGATT | 81 | AGTACGCGCTTAAGATAAATATAATCTGATTAATATTGAGAA | W | A | A | W | GYSNP0244 |
| AATGAATTAGTTACCGGAGATGGGGCCCGAGCACCATTTG | | ATTTTTAGAGTTAACGGTGCTAGATTTTCTATAATCTG[A/T]TG | | | | | |
| GCTGTGACTGCCCACAAGAATGGTCA_NODE_1_length_661_ | | AATTTGTAGGAAGGATCGTTCTAAAGCAGTTGACATTCTAGT | | | | | |
| cov_185.340393 | | GAAGGACTTGAAAGTGTTTTCTGCGTTTAATGAAGA | | | | | |
| RAD_kmer_0000772_1_TGCAGGTGGCAAAGAGGTTGTGGA | 235 | CATTCACAGTTGTATTTCCATCCATACAGCACATCATGACAAT | C | — | C | T | GYSNP0245 |
| ACTGGATGACATTTTCATTTTAGTGGCTCTATAAATTGAC | | CCCATTTTTAGAGGCCTAATTGGTGCATTTTTACAGC[C/T]GTA | | | | | |
| GGGCTATGTGACATTGTTCTTTGCGAC_NODE_1_length_550_ | | AACCACTGCTTAACAGTCTTATTTTACAACCATATAATGCTAT | | | | | |
| cov_113.096367 | | ATATTCCAGCCATAATTCTCTACATAACAGCCCA | | | | | |
| RAD_kmer_0000775_1_TGCAGGTTGATGGCTTGTTTCGAAG | 539 | AATCTTATTGTACATCTGTTCGTCACAAACAAATATGTAATGT | A | G | R | A | GYSNP0246 |
| CAATCTACCAGGGATTTTCGCAGTTGGTGATGTGGCAGCA | | TAATCTAAATGCCAAGAATGTAAGGTGGTAGTTGACA[G/A]G | | | | | |
| TTCCCATTAAAAGTGTGTTTTCTCTA_NODE_1_length_609_cov_ | | TTTCTAGAGAAAACACACTTTTAATGGGAATGCTGCCACATC | | | | | |
| 145.267654 | | ACCAAGTGCGAAAATCCCTGGTAGATTGCTTCGAAAC | | | | | |
| RAD_kmer_0000776_1_TGCAGGAGATATTAATCCCACAAGT | 100 | ATAACATAATTCTAAAACTTTAAGTTGTTGTAGGTTTATTGCG | A | W | A | T | GYSNP0247 |
| AAATTGTTTTTTTTATTCTATATTTTATATTTACTTTTATTA | | AGCTATGGTTTTTTATACGGCAAGAAAAGCAAATATT[A/T]TT | | | | | |
| TATTTATAATTTAATCCGGTTTTT_NODE_1_length_501_cov_ | | GAAAAGAAAATGATTAAAGGTAGGTATATGTAATACAATGA | | | | | |
| 100319359 | | GTTGACGTCAACTCGCGGATTGCGACGAGATATCAAT | | | | | |
| RAD_kmer_0000777_1_TGCAGGAGGTGTGGATCTTAATGTT | 469 | ATATAGTGTGGAGAAAAAAGATCAGGCTTGAGAGGTTCATTC | G | A | G | A | GYSNP0248 |
| CCTTATTTCGAGTATGAACCTTTTCCCGCTCCAGTTAATCT | | AAAGATCCACTGCTGAGGTCATCCTTCTTCTCGGAGCC[G/A]A | | | | | |
| AACAAAGAGTGTTGGTGAAGGGTCT_NODE_1_length_584_ | | ACTCTTTCTTAATCTCAACACCACCAGTAGTGTTCTTGCCTTT | | | | | |
| cov_151.921234 | | GCCAGTAGACCCTTCACCAACACTCTTTGTTAGATT | | | | | |
| RAD_kmer_0000786_1_TGCAGGTTGTCGTAGATCCCATTGG | 188 | CCATGTAATTCGTGAGGAAAGTCATAAATGCAAAAAAGAAA | T | — | T | K | GYSNP0249 |
| TGTGTCCATATGGCAGATGGCTGCTCAACCTTCAAGTAGG | | ATGGCTCATACCCGTCACTACTGCCTGAGTATATTCTTT[T/G]G | | | | | |
| AAGCAGTTCAATCCTGAACCAGATTT_NODE_1_length_594_ | | CCATCTGTACTCCAAGTGACACTTAATATACGACCTGCATAG | | | | | |
| cov_188.796295 | | ATATGATGAATGCACAAATGTATTAATGACAGATATA | | | | | |
| RAD_kmer_0000790_1_TGCAGGGATAGGGAAGACCGCGAT | 91 | GAGGGATTCTGTATCACATATCTTAATCCCCATTCCCATTCTT | T | C | T | T | GYSNP0250 |
| TGCAAAGGCTGCATATGAACGCCTTGCGATACTTTTCAAA | | CAACACTTTGATAGTGTTTCATGTGCACAGCCAGTTC[T/C]TTC | | | | | |
| GCCCGGTATAGCTGTTTTCTTTCAGAA_NODE_1_length_599_ | | AGAACACCAGGACACCCTGCCCCCCAACACACTGCTTTCCTC | | | | | |
| cov_144.263779 | | GACACATCCTCGTACCCTTCTTTCGGGTGGTCTTG | | | | | |
| RAD_kmer_0000794_1_TGCAGGTAAATAGTGTATACTGTAT | 137 | TCTCTTATCACAAGTTCTCAGTAAGGCATTGCCCTTAGGTGGA | C | — | M | Y | GYSNP0251 |
| TACAAGATAAGCAAACACAAAATATAAGATTAATGTTCA | | ATAGTTGTCTAGTGGCTAATCGCTCTCATTTTTTGTC[A/T,C]TT | | | | | |
| AAGAATGTAAAGTGTTTACCACACCAT_NODE_3_length_235_ | | ACAAAAAATAAATGGTTTTCTACATTTCATTATGGTGTGGTA | | | | | |
| cov_269.051056 | | AACACTTTACATTCTTTGAACATTAATCTTATATTT | | | | | |
| RAD_kmer_0000800_1_TGCAGGCTCCAAACACCGCCAACGC | 440 | ATATGGAAGTTCCTTCAGTTAAGATAATATTACATTTCTTCTA | G | A | A | R | GYSNP0252 |
| AATGCCATGGCAGACTGGAATTGTATGTGCATTAAAATGT | | CGCTGAATTAAAAAATAAAGTAATAACTTTTCTCTCT[A/G]TT | | | | | |
| TGTTTACCAAACCATTTCATGGCCTT_NODE_1_length_547_ | | GCATGGAATGAACACTGATTTTGATGTAACAGGGATGTGTCAA | | | | | |
| cov_86.965263 | | GGCCATGAAATGGTTTGGTAAACAACATTTTAATGC | | | | | |
| RAD_kmer_0000802_1_TGCAGGCAATAATCCTATATGTACA | 141 | TTAGTTTGCTCCGTAAACATGTTTTGGATGGCTTAGTGTTATA | T | T | G | — | GYSNP0253 |
| AATATTTAATGTTATGATTCATACCTGCAATAATCCTATAT | | TCGATGCAGGGTTGCAGTTATAACATTACTTAATTTG[G/T]TA | | | | | |
| GTACAAATATTTAACGGTCAGATCT_NODE_2_length_523_cov_ | | ATAAGATTGTTAATACACAGTAATCAGGGCTTTTATATTGACT | | | | | |
| 70.235184 | | TAATTCATAAACTAATAATTTTAATTCTCTTTTCA | | | | | |
| RAD_kmer_0000805_1_TGCAGGAATGTTTTGGAGGCCCACA | 330 | CAACATGTTAAGAGATAAAATGTACCCCAAATTGACCCGCAC | Y | C | Y | C | GYSNP0254 |
| AGCTCGAGAATTGCAGTCTCTAAATCTGAAATTCCATGAC | | CCATATATGGGTCGAAATTTAAAATCATCACTTGTACG[C/T]T | | | | | |
| CCGTAATGGCACACGTGTAAATATGC_NODE_1_length_617_ | | GTGAACCAAGTTTTTCTCTAGAAAAACAGTTTAGCTTGTTGAC | | | | | |
| cov_134.953003 | | TGTTAAAAAACAAACAATCTTGCACCAAATTAACAC | | | | | |
| RAD_kmer_0000808_1_TGCAGGAAGCACGGGTCAAAACCA | 284 | AAGCAATATCTGGAAAAGATGAATCTTGATATACACAAAATG | — | R | A | A | GYSNP0255 |
| CTAGTTAGATTAAAAATAATGATCATTTTAAATACTA | | CATACCAATTTCTCCATCTTTCACAAATTCAAATCCTC[A/G]A | | | | | |
| TTATATGGTTAAAGCTAACGTTTAGGT_NODE_1_length_571_ | | AGAGCATTGATGAGACAACTCCATGATTCAAAAACCGGAAG | | | | | |
| cov_70.455345 | | AAATCATTGATAACTCATTGAAGGAGGGGCAAATTGGG | | | | | |
| RAD_kmer_0000816_1_TGCAGGATCATATTAATATAGTTAT | 132 | AAAATGATCAGTGTAACGCACAAAACGCATGATATTTTTTCTA | C | A | C | C | GYSNP0256 |
| TACATAATCTATTGCTTTGGAAATTTCGGCTGTTTTATTCC | | AACTGCCTGGTTTACATCCTTGAGGGATACGGTCAAAA[C/A]A | | | | | |
| TCAAATATATCTACTATCCCTTCTT_NODE_2_length_395_cov_ | | TGTAATCTTTTATATTGGTCTGGTTTGGATGGGTTGAGCCGAA | | | | | |
| 82.205063 | | ACATTTTTGCCAAAAAAAAACTTTAGGACACACTGG | | | | | |
| RAD_kmer_0000823_1_TGCAGGTTCTTTAATCTCTTTTTCTT | 150 | TGTCAGCATGTGTGTGTTACACCAATCATTGTTGACTTTAGTT | Y | Y | T | Y | GYSNP0257 |
| GTGGCAACGATTAATGCATACCATTGCTAAGGGTCATAAG | | TCAGTTTCACTAGAAAAATACAGGGAACGTAAACAGT[T/C]C | | | | | |
| ATGGGCGGGCTGCGTAATGGGTTTA_NODE_1_length_322_cov_ | | AAAGACACTATATTGGAGAACTCTAAAATTAGTAAATCTAAA | | | | | |
| 22.866459 | | ATAAAAATGTAAGGTTAGAAGTGGAAATTAGATTTTG | | | | | |
| RAD_kmer_0000832_1_TGCAGGGCCAATTCTCCGGTTGCAG | 227 | TCTTCATAGGCTCTCGTGTGTGTTCATTGATCATTAGAGATGC | M | M | A | A | GYSNP0258 |
| TGAGGTTCAATGTTGCACCATAGCTAACGAGATGGTCCCG | | GGCCACCATGGATCTGTTACTGCATTGCTTTGTTTCT[A/C]ATC | | | | | |
| GTTTGCTGACCACACCACTCGAGGGA_NODE_2_length_312_ | | TCAACAACTGCCCAGCAACAGCCTTTCAATTACCCTACTGCA | | | | | |
| cov_44.653847 | | AATCTTTCCACCATATGGGCCAATACTGAATCTTT | | | | | |
| RAD_kmer_0000838_1_TGCAGGTTGGGTATTTAGTGACATC | 499 | AAATTAAGATCATCTGATTTAACCTCACAAGTATACTTCTACA | G | R | R | G | GYSNP0259 |
| TTGCTTGCTCAATGAGAATCATGATTTTTTAAGATTAGCA | | TCCATACCAATGTTAATGCCAAGCACTGAAACGTTTC[G/A]TT | | | | | |
| ATTAATACAGTTCGCAATGATATTAT_NODE_1_length_573_ | | GCGGCCAATAATATCATTGCGAACTGTATTAATTGCTAATCTT | | | | | |
| cov_154.664917 | | AAAAAATCATGATTCTCATTGAGCAAGCAAGATGT | | | | | |
| RAD_kmer_0000843_1_TGCAGGTGGCTTTTTACTTTGACCC | 427 | AAACATATAGGGTCGGGTTGCTAGAAACACTTTTAGTACAAA | — | C | G | C | GYSNP0260 |
| ATTGATTTTAGTTCTAAATAATAGTAATTTCTTGATTATCT | | ATATTAAAGCTTTCTTATAAATGATTAGTGTCAAATAT[G/C]A | | | | | |
| TGATTCCAAGAAAACCTTCTTCTA_NODE_1_length_547_cov_ | | TTACAAGAACTACAATGTTTGACTAATAATCTATATTTTATAA | | | | | |
| 93.914078 | | AGCAACAGATTTAGAAGAAGGTTTTTGTTGGAATCA | | | | | |
| RAD_kmer_0000853_1_TGCAGGGATGGCAAAAATAACAAA | 113 | TCATAGCATAAAAACTACAAATATATAACCTAAAAGGAAAGT | C | C | C | T | GYSNP0261 |
| AAGTGTTTGGAATAAGCAAACCGAAAACATTAGGGCGTT | | TCATTTTCGCATAATGAAATTGTTAGAATGAATTATTA[C/T]T | | | | | |
| AATCAAGTATGAGACTGGATATAATATT_NODE_1_length_343_ | | AGAAAAATGTTTATTACTAGAAAAATGTTAAGAAAAAATAAC | | | | | |
| cov_113.314865 | | AATACTATTTGAAAAGTTTTGTATTTGCTTCGGGTAC | | | | | |
| RAD_kmer_0000853_1_TGCAGGGATGGCAAAAATAACAAA | 218 | AATGTTAAGAAAAAATAACAATACTATTTGAAAACTTTTGTA | G | A | A | — | GYSNP0262 |
| AAGTGTTTGGAATAAGCAAACCGAAAACATTAGGGCGTT | | TTTGCTTCGGGTACTCATGGTATATGATTTCAGGTAAT[A/G]G | | | | | |
| AATCAAGTATGAGACTGGATATAATATT_NODE_1_length_343_ | | TATTTTGTGTCACCTAATAGCATAACCAAAGTTGTACAAGAG | | | | | |
| cov_113.314865 | | CCCGTTTTTTCTTTATCAATATTATATCCAGTCTCAT | | | | | |
| RAD_kmer_0000864_1_TGCAGGATCAAACTTTGGAGTGCAA | 356 | AATACTAATCTACTTTTTCAACTTTCTAGAATAAAATGATTTA | — | T | C | C | GYSNP0263 |
| TTTAAGACGATATCTGTGTTTCCTGCATGGTTTTAGTCATG | | GGAGACCTAGTGCATTAAAAATACACCGAGTGACTTT[C/T]GA | | | | | |
| AAATTTAATAATGAGAACACATATA_NODE_2_length_603_cov_ | | CATTAAATTATTTGACCGTTTTTAGCTACTTCTCTTTTTTATAT | | | | | |
| 81.872307 | | CACATGTTTGACCCATTAGAGATACACCTAACCC | | | | | |
| RAD_kmer_0000864_1_TGCAGGATCAAACTTTGGAGTGCAA | 454 | CCGTTTTTAGCTACTTCTCTTTTTTATATCACATGTTTGACCCA | G | A | G | G | GYSNP0264 |
| TTTAAGACGATATCTGTGTTTCCTGCATGGTTTTAGTCATG | | TTAGAGATACACCTAACCCGAACTGACCCTTTGGTT[G/A]GTA | | | | | |
| AAATTTAATAATGAGAACACATATA_NODE_2_length_603_cov_ | | AATAGGCTCTGTAGTTTACAAATTTTTGAAGGTAATATTAATA | | | | | |
| 81.872307 | | AAGCTGATATATAAGTGTGCGTAACCAAAGTTAT | | | | | |
| RAD_kmer_0000865_1_TGCAGGACTACGGTCAGCCGCTGCG | 526 | GCTTCGCTCGATCAGAAATCCAAACGTAAACCAACTAATCCA | C | T | C | — | GYSNP0265 |
| CTGTTAATTAGTTAGTTAAATTTCCTTCACTGTAGCGGTTG | | TATTTTGCTTGATTTTGCCATCCATATTCCATAGTTTT[C/T]AA | | | | | |
| TGTCGCCGCATTGCATGCCTCTCGC_NODE_1_length_612_cov_ | | TGACACATCCCGTAATCCTGCGAGAGGCATGCAATGCGGCGA | | | | | |
| 74.060455 | | CACAACCGCTACAGTGAAGGAAATTTAACTAACTAA | | | | | |
| RAD_kmer_0000866_1_TGCAGGACAGCTGGCGAAAGACAT | 89 | CTCAACATATTTGGTTGTTTATATAAGGTCGTTGAATTTATTA | W | W | W | T | GYSNP0266 |
| TGAGAAATTATCTGATGACGCTGCAGCTAATTTTGGTTTT | | AACAAGTCCTTGTAAATTTATAAGTTCAAGTACTTGA[A/T]CA | | | | | |
| ATACATCTCAAAAAGATTCTTCCTGAT_NODE_1_length_443_ | | TGCAAAAAACGACACTCTTGTTATACATAGAATCAGAATTTT | | | | | |
| cov_38.291195 | | GTAATTGAGTTTTTATGAGTTCGGAAGTCACATAAA | | | | | |
| RAD_kmer_0000866_1_TGCAGGACAGCTGGCGAAAGACAT | 332 | TTTAGATTATATTGAGATTATAACTTTTAATCAAATTAACGCA | K | G | K | T | GYSNP0267 |
| TGAGAAATTATCTGATGACGCTGCAGCTAATTTTGCTTTT | | ATTATTTATGTTAAAAAGATGAACAAGAAGTTTTTAT[G/T]AT | | | | | |
| ATACATCTCAAAAAGATTCTTCCTGAT_NODE_1_length_443_ | | TCCCTGCCAGCTTTGACATGTACTTAAAAGAACATGTTTCAA | | | | | |
| cov_38.291195 | | GCCAAACCCGTTTTATCCCGTTACCCAATCTGCGGG | | | | | |
| RAD_kmer_0000873_1_TGCAGGAACCACAAGTGCATTTGTT | 319 | CATTGCAGGGTTTCTACTCCATTCAGCAACACACGTGACTTTG | A | R | G | A | GYSNP0268 |
| GATTGTAATACTTCTTCGGAAACTGCACTACTTTTCATCTG | | TACTCTGGTTATGGACCTCACATAATGAAGTCAACAC[G/A]AG | | | | | |
| AATGTTTTGCTTGTCTTTCTCCTTG_NODE_1_length_581_cov_ | | ACTAAGGAAAACCGAAGAATCTCTCAAAACCGGTGACCCCA | | | | | |
| 83.356285 | | AGTTCCCGAAAACAATCTGGCGAATGAAACACCTCTG | | | | | |
| RAD_kmer_0000875_1_TGCAGGAGATGAGCAGATGGGGTA | 139 | CACGCATCCGAGACAGAAAAGGGAAGGCTCAGAAGTAGAAA | Y | S | Y | Y | GYSNP0269 |
| GAAAGGAGATGAAGTTAAGAGGCTAACTTGTTACTCTTTT | | AACTAAAAAATATATAACATACGTAAAATATATTAAAAA[T/C, | | | | | |
| ACGTGTCAAGGGCTGATGGGATGCAAC_NODE_5_length_222_ | | G]ATAATTCTAATTCTAATAATAAATATATGATAATCTAACTA | | | | | |
| cov_70914413 | | TGAAGAATTTCAATTTCAAGAATTAATTATTCTTATACT | | | | | |
| RAD_kmer_0000876_1_TGCAGGCTAGAAATGTAATTATCAA | 361 | CCTCTCACCACCTTGCCATCGCCGTCATCCCCACCACTTCCGC | C | C | Y | — | GYSNP0270 |
| GTACAAAGGTTTCTCAAACCAATCATTTTGGAGAAATTGC | | CGTCGCTGCTGTTCCTCCCCGTCCCTCTCTGTCCCGT[T/C]GCT | | | | | |
| TACCAAACTTAAATGCAACACTAGAA_NODE_1_length_597_ | | GCTGTTGCTCGCCGTCAGTCCCCACACCCGTCGCGTGCCTTCC | | | | | |
| cov_91.432159 | | ACCTCTCCCACAACCCATCTTCTTCAACCTCCCC | | | | | |
| RAD_kmer_0000877_1_TGCAGGAAGCGTAAATTCATCTATG | 339 | TGGGAGCAGCCCAACAGGCTTCCGGAGGAGAAGCTGGAGGA | G | — | A | G | GYSNP0271 |
| CAACTTACTCTGAAGCCTAGAGCTGTAAGGGCAGAATCAT | | GTGGTGGCATGAGAAGAAGCCGTAGAACCTCGATTGACT[A/G] | | | | | |
| CGTTACGGCCTACCAGAAAGACAGAA_NODE_1_length_477_ | | GTGGATGCCGGTGAGACAATTCTTTCCCTCCAACCAGAGACC | | | | | |
| cov_96.798744 | | CTGTATGTCCAACAGACGGCCGTACAGAAGATTCTGTC | | | | | |
| RAD_kmer_0000885_1_TGCAGGCTGATAAATGTTCCTACTC | 333 | ATTAAAAATTCAGAAAGTGACAAGATTCTAAATAATAGTAAA | A | — | A | R | GYSNP0272 |
| TTTTCCTGACTGAAACTTTTATAAATGACAAATTAGTAGG | | TTACAAATGTTTGAACTGGTATACAGCAATAAAATTAT[A/G]T | | | | | |
| CCAAATGCTTTCCACAGAAAGAACAA_NODE_1_length_449_ | | TGTCCTTTCATGGTCTGTGGTACTGGCCTTGTTTTTACCCATG | | | | | |
| cov_151.808456 | | CGAATTTTTGTTCTTTCTGTGGAAAGCATTTGGCCT | | | | | |
| RAD_kmer_0000886_1_TGCAGGGGATAAAAAATTACTGGG | 98 | CTAAAAACGCTTACAAAATCTCAATCAAGAAGTAAAGAAAA | Y | Y | T | C | GYSNP0273 |
| AGCAGCCTGTTGAGGTCAGTGATTCTTTCCTATGAATTAT | | AAGGTAAACTAAGGGGAACACATGTTTGTGTTGTATCTG[T/C] | | | | | |
| AATTGGACAAAACTATCATTCTATGAT_NODE_1_length_468_ | | TAATGTGTGTATCTACCTCCTTTGTTGTTTGCATAAAACACGT | | | | | |
| cov_172.331192 | | CAAAGTTTAAGTACGGTGAAAAAGAAGTCGATAAATC | | | | | |
| RAD_kmer_0000886_1_TGCAGGGGATAAAAAATTACTGGG | 204 | TGTTTGCATAAAAGACGTCAAAGTTTAAGTACGGTGAAAAAG | R | A | R | A | GYSNP0274 |
| AGCAGCCTGTTGAGGTCAGTGATTCTTTCCTATGAATTAT | | AAGTCGATAAATCCAAGTTATATGTAAAGAGAAAGGGT[A/G] | | | | | |
| AATTGGACAAAACTATCATTCTATGAT_NODE_1_length_468_ | | GTCGAATCATTTCCGGCTAGAAAGATAGATCCCGGAAATAAT | | | | | |
| cov_172.331192 | | GGCCACCCCTGCCACCGCCACTCCAACTGTCGACAAAA | | | | | |
| RAD_kmer_0000889_1_TGCAGGACTAGCATCCCTTGGTGGA | 499 | TGCTCCAAAAATTCATCTTAGTTTATTATGAAATGTTTTCTTG | R | R | R | R | GYSNP0275 |
| TGTTGAGGGCTCAAGTGATGTTGACCGCTTGTCACCACTG | | ATTTGCTATTAATCTTTTTACCTATGTTATGCAGATG[G/A]CGC | | | | | |
| ACTCACAACTCGTGTCTGTGGTGACA_NODE_1_length_568_ | | TTGTCACCACAGACACGAGTTGTGAGTCAGTGGTGACAAGCG | | | | | |
| cov_144.028168 | | GTCAACATCACTTGAGCCCTCAACATCCACCAAGG | | | | | |
| RAD_kmer_0000890_1_TGCAGGAGTACCCTAGTGATGCGCC | 299 | CAGCTTTATTCTCCGACAAATATATGTGTGTGAATAATATATA | R | A | G | A | GYSNP0276 |
| TCCTAGTCTCCTACTCTAATTCAGCTACCACAATATAAGC | | TTCATTAAAAGAAAACATAATGATCGTACTGTTTACC[G/A]CA | | | | | |
| CTCATAAGCGTTATTTATATAAAATT_NODE_1_length_576_ | | GAGACTGAAGTAGGTTAAGTGAATAAAAAGTAAAAAATGAT | | | | | |
| cov_102.322914 | | GCTAACAACGTTGGTTCCCCGCTTTCGTGGCTTCCAG | | | | | |
| RAD_kmer_0000893_1_TGCAGGGTACACTGTAGAGTTTATG | 368 | CATACGACCACAGATACTACGAAAAAGTACAATCAAACCACT | R | G | R | G | GYSNP0277 |
| TTCACTTCTTTAGCTGGTTGAGGTGATGGTTTCATGTTCAA | | TTTCACATGTCTGCACCGCACCAATAGAGTCAGAAAAC[G/A] | | | | | |
| CACTCCTGTTAAATGATGGCTTATA_NODE_1_length_456_cov_ | | AGCTTTCGGTTTTAACAGGTGCATGTTTACAAGTTAAGGGCC | | | | | |
| 42.230263 | | ATGGGTTCAAAAAGGTCCTCTTCTTACGCCTTCGGTTC | | | | | |
| RAD_kmer_0000895_1_TGCAGGTAAAGAGTTAGTGAAGGTT | 158 | ATTATTATAGTTACTAAATTTGTTAATTATAAAGTTGAAAATA | T | W | A | — | GYSNP0278 |
| TCCTTTAGCATAGATGCCAATGGCATTCTTCATGTCATGG | | CCCCATATGGCAAAGAAACTTAATAAGAATTATTTTA[A/T]TA | | | | | |
| CTGAGTTAGTATCAACGGGTAATAAG_NODE_1_length_587_ | | ATTATTATATACAGTTTATAATTTGACGGATATTACATTAATT | | | | | |
| cov_71.720612 | | CAGTAAAGAGGAGATAACAAAGTGAAGTTTCCTTT | | | | | |
| RAD_kmer_0000895_1_TGCAGGTAAAGAGTTAGTGAAGGTT | 268 | GGATATTACATTAATTCAGTAAAGAGGAGATAACAAAGTGA | A | A | A | G | GYSNP0279 |
| TCCTTTAGCATAGATGCCAATGGCATTCTTCATGTCATGG | | AGTTTCCTTTTGTATGACCAGAAAGGACCTACGAAAGAT[A/G] | | | | | |
| CTGAGTTAGTATCAACGGGTAATAAG_NODE_1_length_587 | | CAAGAAACTGCTTATAGATAACATACATATCTCATAAAAGAC | | | | | |
| cov_71.720612 | | TGTCAAATATCACATACATAAAAGCAACACATGCTAGA | | | | | |
| RAD_kmer_0000896_1_TGCAGGTTGCAGCCAAACGGCTTGT | 360 | AAGCCAGAAAATATATGAAGGGGTGAAATATATAATTTAACT | A | A | W | T | GYSNP0280 |
| AATGTTTTGAGGTACATTTATCAGCAGAAACTATGAACCT | | GTAGTTTTCTTTTTGGGTCCCTGTTGAAGGGATTGGGG[A/T]A | | | | | |
| TCACTGTTGCAATTATGGATTATGAG_NODE_1_length_624_ | | TGAATTGTGGTGCTCCACTTTTATTCATTTTCTCATGCCTAAA | | | | | |
| cov_149.326920 | | ACCCTGTTATTTAAAAGGATTAATGTTAGTATTATC | | | | | |
| RAD_kmer_0000898_1_TGCAGGCGCTTCGTGGCAAAGAAG | 379 | CAAGACGACTCGCCACCTTGTTTAATTAAACTGGTCAACCCA | G | G | G | A | GYSNP0281 |
| ACGTTTCAGGTGGGCCCTTTTCATGTTTTATTGTTCTACAA | | CCAACCCTTTAACCCATTAATTACAACCCACCAACCCC[G/A]A | | | | | |
| CCCATATATTTATTCGTGTAAAGATC_NODE_1_length_556_ | | TTATCACGTTTAAAACATGATCACTACCCATCAATCCGTTTGA | | | | | |
| cov_124.215828 | | CACATTTACATAAACCGGTTCGATCATGTTCGGGTT | | | | | |
| RAD_kmer_0000901_1_TGCAGGTCAACCGGTTTTGTCCCAT | 301 | AACATAAACTGTATGATCAACAGTGAACCTAATAAGTCCCAG | W | W | A | W | GYSNP0282 |
| ACAGATAGTTACCCCTTTCGACCCATAATCAACCGTCCAT | | CTATATGCTGTGTTTTTGTTCATAATTTGCAAGTCTTA[A/T]GT | | | | | |
| TTTAGAACCTTTAATATTATGCGTCA_NODE_1_length_584_ | | TCTGTTTATTGTTTCCAGAATCTTTCAGCAATTACTTCAGCAA | | | | | |
| cov_173.765411 | | CTACGGAGAAATAACAGATTCTGTTATAATGATGG | | | | | |
| RAD_kmer_0000902_1_TGCAGGGTCCACTCTAAATTATGAT | 246 | GAACCAGCTCCATGAACCCACATTGCCGAGTTCTCATTCTGG | A | G | A | R | GYSNP0283 |
| GACGTGTTGAATGCCCATTACAAAGATAACAGTCAGTTGC | | GAAAAAAAAACCAAGATAAGACAAAAGGGACACGGCAC[A/G] | | | | | |
| TCTCACTTCAGCAGGTATCCTCTTTT_NODE_4_length_398_cov_ | | TACATGTTGAGCAAAGAGTCGAACAAGTGCATGCCAAAACA | | | | | |
| 24.077890 | | GGCAACTTTTGGTATGAAACGAGCTGAGTTGACACGACG | | | | | |
| RAD_kmer_0000909_1_TGCAGGGTTTTTCTCTCACCTTGATC | 117 | ACATGTCTATTTAAAAAAAAATACATATAAAAATGTCTGTAT | — | T | T | C | GYSNP0284 |
| TGGACAATGGTACTTGAAATCTGTACTGTTAGATTTTTTTT | | ATAAATATAAAACTGAAATTACATATAAAATCTCAATC[T/C]G | | | | | |
| TTTTGTTTATTGTTTCAAGAGTGT_NODE_1_length_462_cov_ | | ACTAATCCTGACTAATCCTTATTAATCCCAACTAGCGCTTAGC | | | | | |
| 96.309525 | | AATTCTTACAACACTGGCAGCAACCTTACCGTTGCT | | | | | |
| RAD_kmer_0000915_1_TGCAGGTGTTCTACAAAGTGCCTGA | 357 | TAAAGTCTTGGTTCAGAGTTTTCAGAACATGGCTGCAAGGCG | C | C | Y | Y | GYSNP0285 |
| TACCTAATTGAGTATTCTCATTGAGGTTATGTATTTTGATG | | GGTTGCAGATTAGGACCTGTCATCCACCGTCCCCGCCA[C/T]G | | | | | |
| CTTAATAACAACAGCTTTTTTGAAT_NODE_8_length_485_cov_ | | CCATCGTCATCAGTCGTCGGAAATGATTTGTATTAAATGTCAT | | | | | |
| 25.993814 | | ACATAATCACTTTTGGAATGGAAAAACGGTACTGGA | | | | | |
| RAD_kmer_0000917_1_TGCAGGCTTTGGATGTGGTAATGAA | 108 | TCTCCTTCCTCCTGTTTTCTGGTGGAAGAAGAGACAGATCTTT | R | G | R | G | GYSNP0286 |
| GGCGAATCTTTTCAGGGAAAAGATTTCGGTTGGGAGGGGT | | TAGCCTCCGGGCAGCTTGCGTACCCAACTGCTCCTTC[G/A]GA | | | | | |
| ATGTACCCACGAGTCCACAGAAGGGA_NODE_1_length_661 | | AACCTTCCATCTTCAGCCAAGACACAAAATTCCATAGGAACG | | | | | |
| cov_138.001511 | | TAGTTTGGCCTTTTTTCATTCCCCAATTGTAAACAA | | | | | |
| RAD_kmer_0000920_1_TGCAGGGCTTCTTGATAGGATTGGG | 355 | AATAACAATATTGTTTCCTATAGGCTATCGAAAATCCTGAAA | G | G | A | G | GYSNP0287 |
| CTTAGGGCCCAAGAAAAGTGAAAGTTTAGGACGATAGAG | | AGATGACACCTGCTATGAAGAAATTCATCGAGTATTCG[A/G] | | | | | |
| AAGCTCCCGGGTCCTATATATACATAG_NODE_1_length_509_ | | AGCTTGAATATCAAAGGTACGTACCTGCATGGAATGTTTATA | | | | | |
| cov_43.927307 | | TACACATTCTTTTATTTTTATGCTTATTTAATATTTTG | | | | | |
| RAD_kmer_0000922_1_TGCAGGGTGGTTTGAATAGGCTGAT | 207 | GTACATATTTATAAAAATTGTTTAATAGTTTATCATAACACAT | G | — | T | G | GYSNP0288 |
| TCTTGACATGGTAATATTGGCATCTTAATATTTTATTGTTG | | CTAAAATTTATTTTTCTAAACAGAAGGTTTAAAAATT[T/G]TA | | | | | |
| GATAGGACATTATAAAGCATGCTTT_NODE_1_length_614_cov_ | | GAGATATAGGGGTGCAAACTAAAAAACGAAGGCACTGATGA | | | | | |
| 72.239410 | | AACTCAGATATAGCTCATAGCTGCACCAAATAAAAGT | | | | | |
| RAD_kmer_0000926_1_TGCAGGCGAAGCCTCAGGGAACAC | 126 | CTTGTCGACTGGTAATCTGGTGCGTGTTCCAAACTTCTGCTTT | M | A | M | M | GYSNP0289 |
| GCATTCAACAATCAAGTCAGTATGTGTAACCATATTGTAT | | GTTATTTCTTATAATCCAAGATGAATCTACCCACATA[A/C]AT | | | | | |
| AAAATGCATGCATTAATCACACAAACA_NODE_1_length_593_ | | GTCGTTTTATCAGAATTTAACGATTGAAATGTCATCGAAATGT | | | | | |
| cov_143.209106 | | AAAATCCTTATTCACTTGACTGTTGATAGACACTT | | | | | |
| RAD_kmer_0000927_1_TGCAGGGTTTAGTTTGCACGATGAA | 229 | GGAGATCAAGCCCTTAGGTACTTGTTTCTTCAATTAATAAATA | T | T | G | — | GYSNP0290 |
| TAACGGCAAAAGTAGTAGTAGTGGACAAGCTGCTGATCT | | ATTTTTTTTAGTAAAAGTGTAACATTTGGGGAAAACT[G/T]AA | | | | | |
| GAGTAGCGAATTCGATGCGGATCTGGA_NODE_1_length_622_ | | GATATTTATATTTTTTATTAAGATTAAATATTAACTTCTTGAA | | | | | |
| 2_cov_73.172028 | | AACCAAAAATTTAGCTACTCAAATTTATATTTAAA | | | | | |
| RAD_kmer_0000929_1_TGCAGGTCGTCGTTGACTTCCAAAT | 89 | TTGGAGCATAGATGTTAATCATGCCCAACTTGTTACAAATAA | M | M | C | C | GYSNP0291 |
| CGCCGATTCTGTCCAGTTTCAGAACAACTGTGAGTGATGT | | AACTAAGACACATGGCGACCGCTCCTCCTAAACTAGAA[C/A] | | | | | |
| GATTACTTCTGCTTAGTCTGCCACTG_NODE_1_length_454_ | | ATTGTTGATCATGTTGCATCATTAATTGCTGTAGTGTCATTGG | | | | | |
| cov_51.484581 | | ACTTTAATACTTGGGCATTTCACCCAATGATAAACTC | | | | | |
| RAD_kmer_0000931_1_TGCAGGTAATTTGATGTGTCAAAAC | 161 | TTTTTTACTACCTTAACTATTATTTTTACTACACCATATTTTTC | Y | Y | T | Y | GYSNP0292 |
| TAAAGAAAACTAAAGAGGAGCTAACATCCATTAGAAACT | | ATGTTACTTGGTGTTAATAAGTATATATTTGCATAG[T/C]CAT | | | | | |
| AAAAAAATGCAGATAACTTTTATTTTA_NODE_1_length_459_ | | GAGCTGAGAGAAGGGCAAGAAAGTGAAAAGAAAACAATTGG | | | | | |
| cov_129.755997 | | ATTCCTAGAGGTATCGTATATTTGTATTACCTAAAA | | | | | |
| RAD_kmer_0000932_1_TGCAGGTGGTTGTATTCCATTCAAA | 223 | TTTTTTTTTTTTTTTTCATTTTTAGTCAGGTGTGAATGGCTCAA | — | C | M | C | GYSNP0293 |
| GGACCATTAAATCCCGAATTCCTTGATGTATATGCCCCAG | | CAGCAACTTTTCCAGATGCAACTGGAAGAGCTTTTA[C/A]AAC | | | | | |
| GCATGCTGGGAACGTTAAAGTTGCCA_NODE_1_length_338_ | | ATTTTCTTCTCAGTTAGGTTCCTCTGCTGCGGATATGAACCAC | | | | | |
| cov_74.789940 | | AATGATGCAATAGGTGTGTGCTTCAGTTTTCTGC | | | | | |
| RAD_kmer_0000934_1_TGCAGGCAATAATCCTATATGTACA | 242 | TCTTCGTACACAATGCAAATTTCGTAAACATCTGCTTTGGGGA | — | A | G | G | GYSNP0294 |
| AATATTTAATGTTATGATTCATACCTGCAAGAGCTGGTTA | | TGTTGTGTATTTTGGACGATGGGAAACTACTTATGTT[G/A]CT | | | | | |
| TCAAATACATTACCAATTGTCATCTT_NODE_1_length_563_ | | TCCGTTTAACCAACAACTTGTCATATACGACATGATCAAAGA | | | | | |
| cov_79.428062 | | TTCATTTACGGAGGTAAAGACCTTGACGAGTTTTGT | | | | | |
| RAD_kmer_0000934_1_TGCAGGCAATAATCCTATATGTACA | 344 | GTCATATACGACATGATCAAAGATTCATTTACGGAGGTAAAG | — | A | G | A | GYSNP0295 |
| AATATTTAATGTTATGATTCATACCTGCAAGAGCTGGTTA | | ACCTTGACGAGTTTTGTCCACAAAGGGAGGTTGCAATT[G/A]T | | | | | |
| TCAAATACATTACCAATTGTCATCTT_NODE_1_length_563_ | | TAGAGTATGTCGAGAGTTCGATTTCACCATCAGATATATGTT | | | | | |
| cov_79.428062 | | AGGTTTCCATATGATCCTTTTGAATTCCGTGGTTTTT | | | | | |
| RAD_kmer_0000944_1_TGCAGGAGAGCGGTACCGCAGTTCT | 160 | TCCATATATTCAAATATTTTCCTTTTTTCTTCAATATAATCGTC | Y | C | Y | C | GYSNP0296 |
| CGACGGTCGGGATTAATCTTGTAAGTGTAGAAGAAAGAC | | CAATTAAATAATAAATACCATCATCTTTTAGATTAA[T/C]AGT | | | | | |
| CAACCGATTGAAGATAGATGTTTTTTT_NODE_1_length_581_ | | CCAATCAGAATTCTAAAACAATTCTATCATAATCACTAAATC | | | | | |
| cov_167.290878 | | CATAAATCACAAATTACTAGGGGTCTGTTATGTTA | | | | | |
| RAD_kmer_0000944_1_TGCAGGAGAGCGGTACCGCAGTTCT | 304 | AGGGGTCTGTTATGTTACATATATATATACAACTTCAAGGAC | M | A | C | A | GYSNP0297 |
| CGACGGTCGGGATTAATCTTGTAAGTGTAGAAGAAAGAC | | TCATTATAAACATTCTATAACTTAAATACTATCTTCAA[C/A]T | | | | | |
| CAACCGATTGAAGATAGATGTTTTTTT_NODE_1_length_581_ | | ACCCTAAAAACCCAAATTGTCTCCATAATTAAGACGATCATA | | | | | |
| cov_167.290878 | | TTCATCGATTCATCTCTATGAATCTTCTTAACCTTCA | | | | | |
| RAD_kmer_0000951_1_TGCAGGTTTATGCACACAACTTTAT | 362 | CAAAAAAACAAGGTGTCCGGGCACCCCGGCCCCTACAAAGC | T | C | Y | Y | GYSNP0298 |
| TCCGAGTCCTAGAGGAAATCTACGGGGTTAAGTACTAAGT | | TTAACCAATGATTACAATGAAACCTTTAACATCACCTGA[T/C] | | | | | |
| AGTCAACTGATATTGTTTATTGTCAA_NODE_1_length_422_ | | TTCTTATATCAAATTCTACTAGCACCTTTTTAGTTTGCTTGCA | | | | | |
| cov_30.848341 | | ATTTTTACCGACCTTCAGCATCCTACAAAGAAGTTAA | | | | | |
| RAD_kmer_0000958_1_TGCAGGCGTACAACCAACATCCAGA | 230 | AACGGAATTACATAATCAACAACAACCTCACTCTCTATAAAA | W | W | W | A | GYSNP0299 |
| GCACCTAAGGGTCCTACAAAATACACAACCGCTACTTGAT | | TCCTTCACTTTCGATTTATGCAGCTCTGCATCTGCATC[A/T]AT | | | | | |
| GACTTTGTGATTGTTGACTGGATATC_NODE_1_length_517_ | | AGCCTCCAAATCAGGAAAAACAGCAATCATACCGATTGAGTA | | | | | |
| cov_166.827850 | | CCCCTTGGCCATTTCATGAGATAAACTCTCACCAAC | | | | | |
| RAD_kmer_0000958_1_TGCAGGCGTACAACCAACATCCAGA | 449 | TCATTTTCAACCAAATCCTCCTTTAACTTCAATAACGTAACCC | R | R | A | G | GYSNP0300 |
| GCACCTAAGGGTCCTACAAAATACACAACCGCTACTTGAT | | TCATCACAGATCCTGCCTTCAGCGGGGTCGCGACTACC[A/G]TC | | | | | |
| GACTTTGTGATTGTTGACTGGATATC_NODE_1_length_517_ | | AGATATCCAGTCAACAATCACAAAGTCATCAAGTAGCGGTTG | | | | | |
| cov_166.827850 | | TGTATTTTGTAGGACCCTTAGGTGCTCTGGATGTTG | | | | | |
| RAD_kmer_0000967_1_TGCAGGCTTCTCATGTCTTTGCCAA | 497 | GAACTGAATCTACGTCTTTTTTCCCGAATTCCAGAATGATCTT | A | G | R | G | GYSNP0301 |
| CAACACATCTAACACAGGCACTCTAGTTCACTTGGAAATG | | TGATACATGCAACACTGCATCCGTGTTTGTTTCCACC[A/G]CA | | | | | |
| GGTGATTTAAACTTGCAAATGGCCGA_NODE_1_length_597_ | | AAAAAGCTTGATTTCAAAGATTCTTGAAATTCATCGGCCATTT | | | | | |
| cov_137.639862 | | GCAAGTTTAAATCACCCATTTCCAAGTGAACTAGA | | | | | |
| RAD_kmer_0000970_1_TGCAGGAGTGCCGTCGTTTTTCACC | 303 | CATGATTAAGAATAGACTTTTTTTATATTCAATTTTAACTCAA | — | K | G | R | GYSNP0302 |
| GTGTAATGTGGCAAGAGTTTGGTCATTATCTGTTTGGCTT | | GTTTGAAAAGAATGATTGCATGTTAATCTTGGGTTAA[G/A,T]A | | | | | |
| GTAGAAAACTATAAAACAAACATGTA_NODE_1_length_396_ | | ATTAAGCCTCGCAATGAGGAAGTGGTATACATGTTTGTTTTAT | | | | | |
| cov_110.340912 | | AGTTTTCTACAAGCCAAACAGATAATGACCAAACTC | | | | | |
| RAD_kmer_0000979_1_TGCAGGTACTCATTGTCCTAGTGGG | 173 | GGAGGATCTGCAACAATGTTTAGACACTTCACTGCTTAAATT | A | A | G | A | GYSNP0303 |
| CCCATGTATGGCAGTTCCTTGTGCTTGAACCATCATCAGC | | TGGCTTATTAATGAAATGGACATATGTGGCAATTTTCA[G/A]C | | | | | |
| TAGAGTGGCAAAACAGGGGGTTTGGT_NODE_1_length_577_ | | AATTACCAATAAATATGTCAATTTGGGTTACGTATTACCTCTA | | | | | |
| cov_99.325821 | | ACAGGTCACAAGGACTAAGCTACATATTTTACTAAA | | | | | |
| RAD_kmer_0000990_1_TGCAGGCATACAGGGAGCCAGGCA | 287 | TTCGCCAGAGTTTTGTCAACTTCTTCGTAGGACCCAATAACAG | G | G | G | — | GYSNP0304 |
| TGCTCGATTTCAGATCCCATTTTCGGATCTTAACCTAAACC | | AAAGATCAAGAATCCGACCCAAATTTTCTGATTCCAA[A/G]A | | | | | |
| ACAAACTTCACTTAAACCTTTTCCCT_NODE_1_length_600_ | | ATACTTCACAATGATGACCCGAAACCAGCTCTCTGTGAGCAG | | | | | |
| cov_129.373337 | | CATTTTCATCAGATGAATGCATAACGTGTGAGCTTTG | | | | | |
| RAD_kmer_0000996_1_TGCAGGTCCATAATCCATTTGGACT | 183 | TTAATACTAACCGTAACTACCTAAGTTTCCAGCTATAAGCATC | — | G | R | R | GYSNP0305 |
| CCTCTTGGCCCAGAAGCTGTTAAGGCCCACATCTGATTTA | | AACCAATCAACAATAATTAAAACGCTAGTAAACGCAA[A/G]A | | | | | |
| AAGAAAACGTGATCCAAACACAAAAG_NODE_2_length_473_ | | AAGTTACCAATACGATTAAGAAAAGCTAACTGGTTCAAATAC | | | | | |
| cov_53.059196 | | AAATAACTGTTGAACTAAATCAACACAAGTAGTCAAG | | | | | |
| RAD_kmer_0000996_1_TGCAGGTCCATAATCCATTTGGACT | 238 | ATAATTAAAACGCTAGTAAACGCAAAAAAGTTACCAATACG | — | T | K | T | GYSNP0306 |
| CCTCTTGGCCCAGAAGCTGTTAAGGCCCACATCTGATTTA | | ATTAAGAAAAGCTAACTGGTTCAAATACAAATAACTGTT[G/T] | | | | | |
| AAGAAAACGTGATCCAAACACAAAAG_NODE_2_length_473_ | | AACTAAATCAACACAAGTAGTCAAGATATACTAGTTCGTAAC | | | | | |
| cov_53.059196 | | AGCAAAGAATAACTGAAAATAAGAATTATAAACTGAAA | | | | | |
| RAD_kmer_0001003_1_TGCAGGAAACCCCACATGGCGTACA | 188 | AGCAATGTGCGGGTAACTACGGCTCTAGTTACGACAAATTTA | T | C | C | — | GYSNP0307 |
| TCAGCCAGCTTATGGTGTCTACTGTAAGACAACGTATTGT | | GTGGTTTAAAAAAACAATTATGACATTATAAATTTAAA[C/T]A | | | | | |
| GGTTTAACTAAAAAGGGCATAATGCT_NODE_1_length_601_ | | TATGAAAGGTAGAGTACCTTAAAAGTACTAAAAATTTATATG | | | | | |
| cov_63.158070 | | TAAAATTGTTCTATTCAGTGTTTGTGACAATAGTGTG | | | | | |
| RAD_kmer_0001003_1_TGCAGGA4ACCCCACATGGCGTACA | 346 | GTGTACCTATGATGAATGAAATGTTCGGAATGATGGATGGCT | T | C | Y | — | GYSNP0308 |
| TCAGCCAGCTTATGGTGTCTACTGTAAGACAACGTATTGT | | AGACACCTGTTGTGGAGAAAAGCCCAAAGCGTTACCAT[C/T] | | | | | |
| GGTTTAACTAAAAAGGGCATAATGCT_NODE_1_length_601_ | | CCACCTTACCCAAGCATGCACCATACACCATGCCAACATCGT | | | | | |
| cov_63.158070 | | GAAAGGGGATTTTGAGGTGGGGTCCATCCCATTTCAAC | | | | | |
| RAD_kmer_0001004_1_TGCAGGTGGTAGTGGGGATAATGA | 141 | TTACACCACCCACTACTTTTATAACTTTGAAAATCACTCGAAA | — | T | C | T | GYSNP0309 |
| ATCAAATACTGAGAATATCTCCACATTTCCATGGTCTCTTT | | TATAGAGTCCATAAAACCTCTTAAGTTGCTATATTAA[C/T]AG | | | | | |
| TCACCAAATCTCCCAGACGCCGTATG_NODE_length_556_ | | AAGTTGAAACTTGGAATACTACTACTGAAATCGCGTATTTCT | | | | | |
| cov_121.838127 | | ATTCATCATATATTTGTGAAAAAAAAGTACAAGGCG | | | | | |
| RAD_kmer_0001009_1_TGCAGGTTACAATATCCTTCATTAA | 338 | CTTTAATGATTTTATATAACTTGAAAATGTTTGTTTGTGAGTT | C | M | C | M | GYSNP0310 |
| AATTTCACTAGAACCAAAATACTCAGTTTGGTTTGTTCCA | | GCAGATTAAAGATGGTGTTTGGGATTAGTGAAATAAT[C/A]TG | | | | | |
| AAACAACATAATATACAACAAGAGAT_NODE_2_length_414_ | | TTTTTAATAGGAAAAACGCGTAAAATTCGTACATTTTGTGTTT | | | | | |
| cov_49.429951 | | ACGTTGTGTTAAATTGATGAATTACGTTAGGCTTT | | | | | |
| RAD_kmer_0001015_1_TGCAGGCTTCCCACCTCTGGGTGCT | 140 | CACATGTAAAGATTAAGGAGAATAGAACTTTGACAATCAAA | C | C | Y | Y | GYSNP0311 |
| CACGGAGTAAGTGAAATTATGCTTTTATTTACAGTCATTT | | AATTATAGTTTATGGAAGGGAGAAAATAGAGAAAGTATG[T/C] | | | | | |
| GAAGTTTTAGTTCAAACTGAGTTTTC_NODE_1_length_475_ | | CATACTTCATCAGATATACCAAATGTACGTGTTCTCTTCAAA | | | | | |
| cov_51.378948 | | GCGTGTTCAGAATCAGCTGTTTGATAATCTACAGCTGG | | | | | |
| RAD_kmer_0001017_1_TGCAGGACCCACTTGACCCAAGCAG | 418 | ATTCGTAGCATTCTACATATCATTTTAGAAATTTCTGCAGAAA | G | T | G | G | GYSNP0312 |
| TAACTATGGTTGGATATCGTGCATTTTACAACAAACAAAA | | GAAAACAAGACCTATACTTCATTTTGTGACCGCTTTC[G/T]TG | | | | | |
| GATAATTAAATCTATTTAGAAACTAC_NODE_1_length_524_ | | GTTTTAAGTAGAAAGTTAGCGGGTTGAGTTTTACTCAGTGTA | | | | | |
| cov_90.440842 | | GTTTCTAAATAGATTTAATTATCTTTTGTTTGTTGT | | | | | |
| RAD_kmer_0001029_1_TGCAGGATGCAAGCATATGAAGAA | 100 | CGTTCGCATCAACTCCCGATCACACTGAGGGGGAGTATTAGC | T | — | T | K | GYSNP0313 |
| TGTTAGTAGCCAATAAAAAAATAAATATTTAAATATATAT | | ATATATGGCAACATATAAATTGTATTTAATGTAATAAT[T/G]T | | | | | |
| CGGTTTGGGATCTTTTTGATGAGAGTT_NODE_1_length_473_ | | GAATATATTAGTTGATAGGAAAGTCCATTAGATGACAACTGT | | | | | |
| cov_89.355179 | | ATTTCCTGATGTCCCTTCTTGATTGGAGTCTTCTCAT | | | | | |
| RAD_kmer_0001036_1_TGCAGGTTTCACTTTGGGATGTTGA | 248 | ATTAATCGGTAAATTGCATGTTTCAAATCCTTTCCGAAATATC | T | C | T | — | GYSNP0314 |
| ATCTAGCGCTTGTCCCTCATTCACATCAACAAGTAGTGAG | | ATATTTTCTGCTTTCCCGCATGTTCTTCTACTTAAGC[T/C]AAT | | | | | |
| GTCACGTTGTGGAAGCTGTGGCTAGA_NODE_1_length_532_ | | GCGTTTTGAATATGACTGCAAGTGGGAAAACAATAAAAGTAA | | | | | |
| cov_73.827065 | | GTACCATTTGTTCCATCCTTTTGGAGTTTACTTGA | | | | | |
| RAD_kmer_0001040_1_TGCAGGTGGCTTTATTAGCATGGCA | 330 | CTCCACATTCCATTTGGTCTTGGCATCGTGCAGTCTTCGGTTA | C | Y | Y | C | GYSNP0315 |
| ATATGCCGCTTTTTGGACGTCCTATGTCTGCTCACAATTTC | | ACTTAAGCATGATGAAATGGCTATCTTGCACTGATGA[T/C]GT | | | | | |
| CAAGGGATGTTTTAAGGAAAAAGCA_NODE_1_length_619_ | | GTGTCTTATAACCCATAATTAATTAGTACTTAATGATTACGTG | | | | | |
| cov_155.336029 | | CGTGCCATGACCCATAATTAATTAGTACTTAGCAA | | | | | |
| RAD_kmer_0001043_1_TGCAGGAGATGAAAAACAAAGAAA | 134 | AATGAAAGCTTTAATATATAAGTTAAACTACAGCTTACATAGG | K | K | G | G | GYSNP0316 |
| CACAACTGCAAATAAAGGGCCAAAGAGAGGCAAGAACAT | | TTAATACATGGAAACAAAACATGAAAATGTCAACCCAA[G/T] | | | | | |
| GTGCAACATTGTAACAACACTGTCTACC_NODE_1_length_606_ | | TTACCGTGTGTCGTTGTTATGCCTAGCCAAACATCTAACCATC | | | | | |
| cov_94.120461 | | ATGCATTACATCATATATATAAAACAGTGTGATCAAA | | | | | |
| RAD_kmer_0001051_1_TGCAGGTAACTCATAATTAGCTATA | 113 | TTAGCTTAAAAGGAACAATGCCAAATGGGTGTTAAAGCCTTC | M | C | M | A | GYSNP0317 |
| GGTTAATCAGATAATCTATAATTTGAAAACAAGAAGCTAT | | TAATGTGTAAAAAGTCGTAAACAATATTATTCAAAACA[C/A]T | | | | | |
| TATAATGCTAACGTTTTCTATAATCG_NODE_2_length_598_ | | AGATTATTATAGAAACCATATAATTGTGTAATTATATATGAC | | | | | |
| cov_117.454849 | | ATACAAATTATTACTAAAAATCTTAACATGAACAAAA | | | | | |
| RAD_kmer_0001051_1_TGCAGGTAACTCATAATTAGCTATA | 196 | GATTATTATAGAAACCATATAATTGTGTAATTATATATGACAT | Y | T | T | C | GYSNP0318 |
| GGTTAATCAGATAATCTATAATTTGAAAACAAGAAGCTAT | | ACAAATTATTACTAAAAATCTTAACATGAACAAAAAG[T/C]GT | | | | | |
| TATAATGCTAACGTTTTCTATAATCG_NODE_2_length_598_ | | TTCTGATTAACCTGACCCAGCCAAACAAACCAAAAAACGCGG | | | | | |
| cov_117.454849 | | CCCAAACCAACCCAGAAAACACCATTTTGATCCATT | | | | | |
| RAD_kmer_0001053_1_TGCAGGTCAAAATATGAAAATTAGC | 449 | CCCGCTTACCCGCCCTTTTTTGATTAAAAAAAATACATACATT | A | G | A | G | GYSNP0319 |
| CCGTTTGACTCAAATAGTTAAAAATGTTCATCTTCATTTGA | | CTTATTCAGGTATGCAGAGCAGCTTGTTGGCACTTGC[A/G]TT | | | | | |
| CTACAAAATGTATAAAATTCAACTT_NODE_1_length_570_cov_ | | AGCGAATAACTTCTTTAAAGATCCGCTAGTGAGCATTCCTCC | | | | | |
| 130.871933 | | AGCAATATCAGTAAGTTGAATTTTATACATTTTGTA | | | | | |
| RAD_kmer_0001058_1_TGCAGGTTTTTAAAGTATGAGAATA | 250 | ATGGGAATGGATTATACTTTCTAACACAACCTTAAACTCATA | C | A | C | A | GYSNP0320 |
| TCGTCTTGGAATCAAATATGGTTTAAACTAAAAAAATGTG | | TGCTTAATTCAAAACCGGCTAAATAACTACCTCTTGAT[C/A]T | | | | | |
| TCTGTATGTGTAGAACCCACCTTTGC_NODE_1_length_557_ | | TGATGAAGTAAAACAGTTATCTAAAGCTCAAAAAAAGTGGTT | | | | | |
| cov_148.012573 | | GTTTTGTGTTTTTTCTGACATTTTTTTTTTGTCTTAA | | | | | |
| RAD_kmer_0001060_1_TGCAGGTATTACACACGCCCTCAAA | 396 | CCCCTTGTTTATAGTTTTAGCATGTAAATGTGAGAACCAGGTA | — | T | T | T | GYSNP0321 |
| TAGTCACATGGTCTTTCCATCTAGAAAAAATGAAGTGGCA | | GCCTTGTTTCATGTATCGGTACGATCAAGCTGTACAG[G/T]TT | | | | | |
| ATGATGTACCTTTATTAAAAATAAAT_NODE_1_length_543_ | | TTAGTGAGCTCGAGTGTTTCTGTTAACGTGTTAATGTTGTGGG | | | | | |
| cov_95.416206 | | TCCATATGGTTTGCTCTGGAGAACTCTCCTTTTTT | | | | | |
| RAD_kmer_0001063_1_TGCAGGCAAAGAAAAAATGAAGGT | 126 | GCCCGATTGGTGCATGTACCCCTTGTCTTTTGGCTATCAACGC | C | C | Y | — | GYSNP0322 |
| TTGCTTTAGCATAGATGCCAATGGTATTCTTGATGTCTCAG | | CACCACATGATGCATGAATAACCTTCCGCCGTTTTAA[C/T]GT | | | | | |
| CTAAGTTAATATCTACTGGTAATGAA_NODE_1_length_502_ | | TATTTTCACGAAATTAGTAAAATAACGTTAAAATTAGGGCGA | | | | | |
| cov_136.764938 | | TTTTACTTTTGCCCCCAAGGGCCCACACATATATAC | | | | | |
| RAD_kmer_0001063_1_TGCAGGCAAAGAAAAAATGAAGGT | 199 | GTTTTAACGTTATTTTCACGAAATTAGTAAAATAACGTTAAA | G | A | R | — | GYSNP0323 |
| TTGCTTTAGCATAGATGCCAATGGTATTCTTGATGTCTCAG | | ATTAGGGCGATTTTACTTTTGCCCCCAAGGGCCCACAC[A/G]T | | | | | |
| CTAAGTTAATATCTACTGGTAATGAA_NODE_1_length_502_ | | ATATACAGATATATCTGAAATAAAACCTTAAATGTTTTTTATA | | | | | |
| cov_136.764938 | | TATAAACATGAACCTCAAATAAAACCTTAATAAATA | | | | | |
| RAD_kmer_0001066_1_TGCAGGTTTAATTATAAATGTATCT | 126 | TAATCGAAATCAAGAGAGCTAGAATCTTCACTTCTACAGTTC | A | T | A | T | GYSNP0324 |
| TATTAATCTTCTAGGTTATGCGATGTTGAGTCGGTTGAGCT | | TATAGATTCAAGGTTCTATTATAGATAAAACGTAATTA[A/T]A | | | | | |
| TCGGTATGGGATCATCAGATGTAGT_NODE_1_length_617_cov_ | | GTCTTGTATTAATTATGTCTAAACTTCGAATCCTACACCATAC | | | | | |
| 103.207458 | | CATCTTGCCTTTCCGCCATACGTAAGATTGGCTAAA | | | | | |
| RAD_kmer_0001067_1_TGCAGGTTGATTGTAAGTGGGTGTA | 212 | GTCGGTTTGATAATAAAATCCACCTTTTCCTCCAAAATAAAGT | G | C | C | — | GYSNP0325 |
| CAGGTTAAAGCGGGATATATGCACATGGTAAAGTTACGT | | ATTCTTGGATGAAAGATGCATCTGTTGAAGAGCATAA[C/G]G | | | | | |
| ACTCGCTACAAAGTTCATCTTGTAGCC_NODE_1_length_633_ | | ATTGCTTCAATAGATATCCTAACAAGAAAAACAAACTACAAA | | | | | |
| cov_87.445496 | | TCCTTCATGTACCTCTTGTATGTTACTACGCACAGAC | | | | | |
| RAD_kmer_0001080_1_TGCAGGGCAGCTGGCAAAAGACAT | 346 | GGGATCCTTAGCTTCTCATATAGCTCGTGAGGCTTCCCTACGA | K | T | G | T | GYSNP0326 |
| TGAGAAAATGTCGGATGAGGCTGCAGCTAGTTTTGCCTTT | | TATTATAGCTATATGAACCTAGTGAATTTTCGTCAAT[G/T]CC | | | | | |
| TCACAGCTTAAAAATATTCTTCCTGAT_NODE_4_length_416_ | | CCAATGTGAAACCAGATACTGAATCTGCATAAAACAAGACAC | | | | | |
| cov_26.519230 | | CAAGAAAGAAATTTTTTTGAGGGTCTAGTCATTTAA | | | | | |
| RAD_kmer_0001085_1_TGCAGGTGTTTCAACCTGCTCCACA | 151 | CCAGGTTCAGAGGTGGGTCTGCCGGACTGGCCGGTTTGACTG | G | R | G | — | GYSNP0327 |
| TGGGCTTCATTTTCAATGGAGTTTTCAGCTTCTTCTTCATG | | GTAGAATTGATTGTTTTATTTTTCAGAAATATTAGTTC[G/A]TG | | | | | |
| CTGCMAAATAGAAACCAACTTACA_NODE_3_length_479_ | | AAAATTTAACTGATAAAGATGGTTTATGATCATAATACATAT | | | | | |
| cov_37.048016 | | ACATAACTACATACTCAATACATGATAATTGATAAA | | | | | |
| RAD_kmer_0001086_1_TGCAGGCTAAGCCCATTGAGAATTT | 90 | AATTCATATTGCGCGGAAATTAAAACCCCAACTGTTTGATTA | C | A | M | C | GYSNP0328 |
| CTTATTGATCTTTTGGTCCAAACTCAACTATTTAATATTTT | | ATGTGTCCAGTGAGAGTTTCTGATGGCTTTTGATGTCA[A/C]T | | | | | |
| CTGTCAACATTCTTGACAAAATGTA_NODE_1_length_497_cov_ | | GAATAAGCATAGTGCTAAGAATCAGCTTTCCTGTCATCAAGT | | | | | |
| 64.370224 | | TTTAATTAACTTATATAAAGATGTTACAAAAGCAATT | | | | | |
| RAD_kmer_0001089_1_TGCAGGCTTAATATTTTGTTTAATAC | 161 | GCTCATTGTACAATTTTACTCTACAGTGGTCGATTTCAAAAAA | C | S | S | C | GYSNP0329 |
| TATTTTTTTTGTTGTATAATTCAAACATAAATGGACGAAA | | ATGCACGCATCATGCATTCATGCGAGCGCATCAGGAG[C/G]G | | | | | |
| AATTATATTGGCAAGTTGATTCGAT_NODE_2_length_482_cov_ | | CATTACGCGATTTAGAGCGCATCATCCTCGCATCACGTAATA | | | | | |
| 35.348549 | | GCTGCACATCGCATCACCTGATGCGATCTCATCAGAG | | | | | |
| RAD_kmer_0001094_1_TGCAGGGTTATGGTAATAGAGATGG | 240 | TATAGGATTTTAGGATCTATGGGTTCTGATCACTTGATCTTTG | T | W | T | W | GYSNP0330 |
| TAAATGAATGAACTGGATAACAGGTCAAAGCGAGATACG | | GTACGTCTTGAGCCCATTAAGCTCGACACATCTAAGA[T/A]TT | | | | | |
| TCGAAATATGATTTGTCCAAAAAAAAA_NODE_1_length_617_ | | CAGATGGTTAGTCGAGTCAATTTGACTTGTTTATTCCCATGGT | | | | | |
| cov_112.348457 | | TGAAATGGATATTGCAGAGCAGAGCTGTCCTTTTT | | | | | |
| RAD_kmer_0001094_1_TGCAGGGTTATGGTAATAGAGATGG | 562 | GAATATAGTCATTGAAATAAAAATATTGTAACTATATCTGAC | G | K | G | K | GYSNP0331 |
| TAAATGAATGAACTCGATAACAGGTCAAAGCGAGATACG | | ATTTAAATTATATATAAAAAAAATCTAATTTTTTTTTT[G/T]GA | | | | | |
| TCCAAATATCATTTGTCCAAAAAAAAA_NODE_1_length_617_ | | CAAATGATATTTGGACGTATCTCGCTTTGACCTGTTATCCAGT | | | | | |
| cov_112.348457 | | TCATTCATTTACCATCTCTATTACCATAACCCTGC | | | | | |
| RAD_kmer_0001095_1_TGCAGGAAAATCCAGATTTATGGAA | 193 | CTCAAAAACCATGAGGGCTTTTATACTATAGCAGCCTTATTCT | C | — | C | G | GYSNP0332 |
| ATGGCTAACCGGGCAGGAACAACCTCCCGAACCAATCAA | | TGGTGCTTATAACATATGAAGTAAACTTATTCGTAAA[C/G]AG | | | | | |
| CACAAATCCCGTGAGTTACACATCAAC_NODE_3_length_277_ | | AGTTGTATATATATAATTACTGATTACCGACAATCGGGCTATC | | | | | |
| cov_54.646210 | | GCGACCTTTTTTAAAATCATCCCATCCTCTGACCC | | | | | |
| RAD_kmer_0001099_1_TGCAGGAGACTTTTTCAATGCATCA | 146 | GGTATGGAGCTGGTGAAGGCAGGGTAGAATTTTCAAGTGGA | C | C | M | M | GYSNP0333 |
| ATAGCCGAAGCAGTATCAGGATATTCATTTTCACCATTAA | | GAAAGTTCCGGATCTAGCAGTGGGAAAATGGACCCCACT[C/A] | | | | | |
| CATTCACAGAAAAACTCTTGAGGTAC_NODE_1_length_686_ | | ACGGTCTACAAATTGTTGGGATGAGTGCCACCTTGCCCAATG | | | | | |
| cov_131.437317 | | TTGGTGCAGTTGCTGACTGGCTTCAAGTAAGTTAAAAG | | | | | |
| RAD_kmer_0001099_1_TGCAGGAGACTTTTTCAATGCATCA | 281 | CTGACTGGCTTCAAGTAAGTTAAAAGAAAACATTATAATTTT | W | W | T | W | GYSNP0334 |
| ATAGCCGAAGCAGTATCAGGATATTCATTTTCACCATTAA | | TGGTTTACTATAAGCTGATTGTTGATACAGAATTAATA[T/A]C | | | | | |
| CATTCACAGAAAAACTCTTGAGGTAC_NODE_1_length_686_ | | TTTCAGGCAGCACTATACCAGACAGATTTTCGACCTGTTCCCC | | | | | |
| cov_131.437317 | | TTGAGGAGTATATTAAAGTGGGTAACTCAATTTATA | | | | | |
| RAD_kmer_0001103_1_TGCAGGTGTGGCTTCATTAACAGAC | 521 | GTTAGAAGGTTTGTAAAATAAACAAGCTGGTAGCTACTATGT | Y | M | Y | Y | GYSNP0335 |
| ATTGAAGATCTTCGTCCTTAATTAGGTTTATGGGTTGAGA | | TAATCCTTCCAAAATTCTAATTTCATAAACATGTGTAC[C/A,T] | | | | | |
| TTCTGTGTATGCCAAGCTGATTGTTG_NODE_1_length_645_ | | ATAACTAAAGGGAGATAAAAGTCATTGAAAATTTTGGGGTAA | | | | | |
| cov_131.268219 | | AAACCAAAAATCAATTACAACAATCAGCTTGGCATACA | | | | | |
| RAD_kmer_0001105_1_TGCAGGCCCAACCTTTATACTTTTCT | 100 | ATTGATCACTGATAATTGAGAGTACATGAAAGAAAGCAACA | C | — | C | A | GYSNP0336 |
| TTTTAAAGTGTGTGGAAACATGAAATAGCCCGATTGCACG | | ACCAAGACTCCTATAAATAACAAAGCAACAAACGGCTAC[C/A] | | | | | |
| ACAAGCAGCAGATCTCACAAATGGG_NODE_1_length_568_ | | TCCTAATAACTAGTAAGATCCAAAATACCCGGACAACTCAC | | | | | |
| cov_136.950699 | | AACAGCCTCAAACGGCTTTATTGAATTCAAAGTAACAAC | | | | | |
| RAD_kmer_0001107_1_TGCAGGCGAAAGAAGCACTACAGG | 244 | GACCTCTTTTCAAACTAAGCACGAACAGCTTCAACGATTCTC | K | T | T | K | GYSNP0337 |
| GCAAAGAAGATATCCAGATGCCAAATTTTGAAGTGAGTG | | GAAAAGTAAAACTGTGTGGTAGTAAGACCCCTCTTTCT[T/G]A | | | | | |
| ATTTAGAAAGCTTGAACGGGAAGTATGA_NODE_1_length_ | | TTTTCCAGCCAACTTTTTGTAACGCGCGTTCCACATCAGCTTC | | | | | |
| 380_cov_50.465790 | | TGAGTGAAGATATGCTCTTGTTGCCTTTGAAGGTCC | | | | | |
| RAD_kmer_0001125_1_TGCAGGGTGATGCAGAGAGACGTTT | 117 | GAATCTCCTAATTGTCTATTGAACATCTCAAAATAAATTATAC | T | T | W | W | GYSNP0338 |
| TGATGGAGTTAACCGTGGTATATCAACCTGCATAATTAAT | | CAATTACCATCCATCTTCAAATGTAGACCTACAGAAC[T/A]GA | | | | | |
| CATGATAAACTTGATTATGTGCTAAT_NODE_1_length_638_ | | AGCTAAAGCCACTCAACTCCTTGTTACTAAAGGGTTTGACTTC | | | | | |
| cov_125.206894 | | CGTGGGAAACAAGGATCAGTGTCCAGAATTGCAAG | | | | | |
| RAD_kmer_0001130_1_TGCAGGTTGTAGAAAAGAGCTTTAA | 127 | AAATACTTCTCAAGACTTGAAAAATATCATATTTGCCTAACA | T | Y | W | T | GYSNP0339 |
| AGATCTAGCGTACATCCGTGTGAATGATGGTTCATGTTCT | | AAAAAACATAATATCAAATATACCCATTTTTATATTTA[T/A,C] | | | | | |
| CAACAAATTATCGTCTACACAGACTC_NODE_1_length_503_ | | GACATATGTACCCCTCCGTTTATCTAAGTATTTAGATTATTAG | | | | | |
| cov_126.379723 | | AGTTTGAAGTGTAATGAGAAGGAAAAGTGTATGCTAG | | | | | |
| RAD_kmer_0001132_1_TGCAGGCGGATCATACGATTCATAT | 486 | CCACCCAAACCAAGCCCGGGAAATAAAGGTGCACCAGTCCC | W | W | W | T | GYSNP0340 |
| GGTGCGTGGTTCTGCTCCACCGCCGACTACTGATGCACCT | | CACACCACCTAACCCAACCCCTTCATTCTGACCAGCACC[A/T] | | | | | |
| GGAGCTGGAACGGCTAATACAGCTAA_NODE_1_length_563_ | | TGGGAGGGAGTATTAGCTGTATTAGCCGTTCCAGCTCCAGGT | | | | | |
| cov_97.341034 | | GCATCAGTAGTCGGCGGTGGAGCAGAACCACGCACCAT | | | | | |
| RAD_kmer_0001140_1_TGCAGGGTTATTATTGGGGTCCAAG | 308 | TTGATATAAAGTTACAAATTTTGAACATAAAGTGTTTTTGGTC | T | Y | T | Y | GYSNP0341 |
| AGAGTATGAGAGGATGCTTATATCAGATTATATCGATGCA | | AACCCATCCCAACCAGCTTTTTACTTGTACCAAACCT[T/C]TA | | | | | |
| CCTTGTTTGTCTCTTTATCTTAATGG_NODE_1_length_502_cov_ | | ACCGTTAACCAACCAGCCCATTTTGCCACCTCTAAATTCAGTG | | | | | |
| 184.796814 | | ACGTACTAATGCTCAACAGTCTACATCTACACTAC | | | | | |
| RAD_kmer_0001140_1_TGCAGGGTTATTATTGGGGTCCAAG | 375 | TTGTACCAAACCTTTAACCGTTAACCAACCAGCCCATTTTGCC | K | K | K | K | GYSNP0342 |
| AGAGTATGAGAGGATGCTTATATCAGATTATATCGATGCA | | ACCTCTAAATTCAGTGACGTACTAATGCTCAACAGTC[T/G]AC | | | | | |
| CCTTGTTTGTCTCTTTATCTTAATGG_NODE_1_length_502_cov_ | | ATCTACACTACAGTACACAATGTTAAAAAGATGTTAGGCTAT | | | | | |
| 184.796814 | | ATGTTTAGAGAAAACTTACCATTAAGATAAAGAGAC | | | | | |
| RAD_kmer_0001141_1_TGCAGGAATAGGCTACAGGCCTACA | 100 | AAGAAAATAGGCAAAAACAAAATACCTATATGCAGGAAAAA | A | G | G | G | GYSNP0343 |
| GATGCAATGATGACTCCGTTTAGTCCCAATCACGTGGGGC | | GTACCTTGGCTTGGAAGAGAGGTGTACGTTTGCAGCTCC[G/A] | | | | | |
| CTCATTCAGCCTCCACACTCTATATG_NODE_1_length_630_ | | ATAATCTTTCCTTTTTATCATTCTTTGGTCATAAGTGGCAAAG | | | | | |
| cov_106.209526 | | CAGAGCTCTCATGGAGCTAGTCAATTGTTGGGACCAA | | | | | |
| RAD_kmer_0001141_1_TGCAGGAATAGGCTACAGGCCTACA | 36 | CCCTGGCCAGCTCAGATTAGGTGTAGCGATTCGAAAGCTCTG | G | T | G | G | GYSNP0344 |
| GATGCAATGATGACTCCGTTTAGTCCCAATCACGTGGGGC | | GCAGGGTGCAGGCATGGCCCAAGCCCCTCAGCGGGGCC[G/T] | | | | | |
| CTCATTCAGCCTCCACACTCTATATG_NODE_1_length_630_ | | GGCTAATCCAAAACTTGCATCAGATTGCTGCTGCTGAGACTG | | | | | |
| cov_106.209526 | | CAATGCCATGTCATCAAGAAACATTATTTGCAAAATAA | | | | | |
| RAD_kmer_0001152_1_TGCAGGGAGATCTTTCTTTGCCAAA | 115 | GGGGCTGAGGAGGGTAAGATGTAGACAACCTTACCTCTACCC | C | T | T | C | GYSNP0345 |
| TGCAACCTGTTTTCAAGCAAAAAAAATAATAATCTTAATT | | CATGGGAATAGAGAGGCTACTTCCAGTGAGACCTCTGG[T/C]T | | | | | |
| CTTAACAAGATTAAATATGATGCATA_NODE_1_length_659_ | | CGATATAATTTTGCACAAAAATAATATATTTTATTGTCAGTGC | | | | | |
| cov_106.511383 | | TTCCAGAACAGATTCGGGGAACAAATATTACAATTC | | | | | |
| RAD_kmer_0001174_1_TGCAGGCACAGTTTTCAGGGAACCA | 521 | AATGCATATTGGCTTCGTCCATCCTGCAAGTAAAACCCATTA | A | W | A | W | GYSNP0346 |
| ATTCTCTGCAGAAACATCCCCAGGTTGATTCCAGGTAATT | | GAAATCCATGCAACAAAAGTTTTAGAATTAAAAAAAAT[A/T] | | | | | |
| ACCAATTTTCGTTGTGTTATCTATTT_NODE_1_length_585_cov_ | | AATAGATAACACAACGAAAATTGGTAATTACCTGGAATCAAC | | | | | |
| 133.753845 | | CTGGGGATGTTTCTGCAGAGAATTGGTTCCCTGAAAAC | | | | | |
| RAD_kmer_0001187_1_TGCAGGTTCAGTCCATAGACTTGTT | 124 | TGCTGGACGTCTGTTTCTTACCTTTTTTGCTTTGATTGAAGCTA | C | M | C | A | GYSNP0347 |
| TTCTTTTGCACTTACAATCCCTAAAACCTACCTACTCCATT | | AACAAGCGTAGATAATAAGGGTTATTTTAGTTAAAT[C/A]AAC | | | | | |
| ATAATCAAGCCCATTACTCTAATGC_NODE_2_length_342_cov_ | | TCGCATTTCATATTTTTATGCGGTGGTGTGTGTAATGTAGGAT | | | | | |
| 21.146198 | | GACTTTGGATGAGAGATCAGGACACACTATGTAC | | | | | |
| RAD_kmer_0001193_1_TGCAGGAGCTTTCAGACCTTAAGAA | 251 | AATTTTGACCCGTTATCAATTTGGTTTGTGTTCTTAAACAAGT | G | G | R | A | GYSNP0348 |
| AACTCTTAATGTGGAAGTAGACCAGCTTCCTATCCGTAAGA | | CATATAAATTAGCTAAAAAAAGTCAAATGAACCAAAC[A/G]G | | | | | |
| ACTTTTGAACTCCCTTCCAATCTAGT_NODE_1_length_585_cov_ | | GTGATATAAATTAGCTAAAAATCAAATGGACCAAACAGGTCA | | | | | |
| 97.170937 | | AACAAGTTTAAAGTTGTTGAAACTATTATCGTAAATA | | | | | |
| RAD_kmer_0001193_1_TGCAGGAGCTTTCAGACCTTAAGAA | 341 | AATTAGCTAAAAATCAAATGGACCAAACAGGTCAAACAAGT | T | T | Y | C | GYSNP0349 |
| AACTCTTAATGTGGAAGTAGACCAGCTTCGATCCGTAAGA | | TTAAAGTTGTTGAAACTATTATCGTAAATATATAGTTTG[C/T] | | | | | |
| ACTTTTGAACTCCCTTCCAATCTAGT_NODE_1_length_585_cov_ | | GTATCATAACTAACACATTTATTACTTATAATCTCTAGAAGAA | | | | | |
| 97.170937 | | TGGACAAAAAATGTTACCTACACAACATTTACTAAAA | | | | | |
| RAD_kmer_0001193_1_TGCAGGAGCTTTCAGACCTTAAGAA | 522 | ACCCACCCAAACCCATTTTGACCCGGTACACAACTTGCCCAT | C | T | C | C | GYSNP0350 |
| AACTCTTAATGTGGAAGTAGACCAGCTTCGATCCGTAAGA | | CCTGATACCATTTCTTTAGTTAGCATACAAATTCAGCA[C/T]T | | | | | |
| ACTTTTGAACTCCCTTCCAATCTAGT_NODE_1_length_585_cov_ | | AGATTGGAAGGGAGTTCAAAAGTTCTTACGGATCGAAGCTGG | | | | | |
| 97.170937 | | TCTACTTCCACATTAAGAGTTTTCTTAAGGTCTGAAA | | | | | |
| RAD_kmer_0001196_1_TGCAGGGGTCTTTAGTGGCATTTAT | 379 | ATCAATTTGGATCGCCGCATAAAACCTCGGGAGGAGACAATC | K | G | T | T | GYSNP0351 |
| TTGGCTTTTGTGGCACTTTGTGTATGTCACTAAATAGCCTT | | GGCCCTGCCTTGTTTGTTCTTATGAAAATAAAATGAAA[T/G]A | | | | | |
| TTTTGTAGTGTCGGAATGGTAGTTT_NODE_1_length_598_cov_ | | AAAGTCGAGAACTATTCTGATCGCACTAAGTGTTACACCTCC | | | | | |
| 73.244148 | | GATTATGAGTGTAAGTGTGAGTGAGCTGCGTAATGAA | | | | | |
| RAD_kmer_0001197_1_TGCAGGGGCTGGTGGAACCCCATAC | 244 | CATGTTGAAAACTGCTTAAGAGATGGTAATATACACAAAGGT | C | A | A | A | GYSNP0352 |
| AGAGTGAATCCATCAAGAAAGATGTTGTCTTTGACTTTGC | | AATATAGATGATGTAGTCCTTGTTGGTGGGTCAACTCG[A/C]A | | | | | |
| TGCATTCACCCTGATACACCAACTCT_NODE_1_length_599_ | | TTCCTAAGGTTCGCGAAATGTTAAGGGAATTGTTTGATTGGA | | | | | |
| cov_90.368950 | | AACCACTTTGCCAGAACATTAATGCTGATGAAGCAGT | | | | | |
| RAD_kmer_0001197_1_TGCAGGGGCTGGTGGAACCCCATAC | 334 | GTTCGCGAAATGTTAAGGGAATTGTTTGATTGGAAACCACTT | T | C | C | C | GYSNP0353 |
| AGAGTGAATCCATCAAGAAAGATGTTGTCTTTGACTTTGC | | TGCCAGAACATTAATGCTGATGAAGCAGTTGCTTATGG[C/T]G | | | | | |
| TGCATTCACCCTGATACACCAACTCT_NODE_1_length_599_ | | CAGCAGTGTTGGCTGCGAACTTGTGTGGTAATGGGAATAACA | | | | | |
| cov_90.368950 | | CTGTGAAGGATTTGGTACTTATAGATGTGACCCCTTT | | | | | |
| RAD_kmer_0001201_1_TGCAGGTAAGATTCAACTTTGTTGT | 165 | TTCTTATAACTTGTTGATTCATGTATCTCTTTTTGGCATCATAG | A | G | A | G | GYSNP0354 |
| TAGTTGTTCCATTTACTTTGTATGTTACATTGGAATCGGTA | | CTAAGCTTGAATTTTCATAAATTCACAGTCTACATT[A/G]AAT | | | | | |
| TTACGTTACGGTTTTATATATGCCC_NODE_1_length_594_cov_ | | GGAAATGTTATCACAATCCAAACAAAAAACTAACAGGTAAT | | | | | |
| 117.395622 | | GAACCTAATCACATATCTAGTGACTTTTGCTATCCA | | | | | |
| RAD_kmer_0001205_1_TGCAGGTCTCGGGAAAACCTTAACA | 91 | AATTATTGACTTTTAACTGCTATGAAATATGCAGTTACCTTTA | A | — | R | A | GYSNP0355 |
| GCCTTTGCTTGTCTAGCTGAAATTTAATTCACCAATAAGT | | TTTTTGATTTAGGGTGCAAATGTTGAAGATTTCATGT[A/G]AA | | | | | |
| AATTTTAATTGTAACATAGACAATGT_NODE_3_length_403_ | | TCAAAAACCTTGAATCACTTCTTTCCTTATTTTTTTTAGTTCAA | | | | | |
| cov_81.059555 | | ATTGTAGATTCTATTTATTTTATACCCTTCATAT | | | | | |
| RAD_kmer_0001207_1_TGCAGGATGAATAATAAAACAAAC | 271 | TCATCTATACAAAAACCACCTCGAATGGTGTACACGAGCTTG | — | C | T | C | GYSNP0356 |
| AAGTTCAACTTATAATGATGATTCAAAGACAAATAACATA | | TCCGTCACATCATACGGATTGTATGATAGGGCGAAAAC[T/C]G | | | | | |
| CACTGACTATAAGCTATTTATATCAGT_NODE_1_length_530_ | | GTCATACGGGCCGTATAACTTTGTAAGTCACATGAGTCATTTC | | | | | |
| cov_85.258492 | | TTTGTTAGTCAAAGACGTCATACTGGTCGTTTGATT | | | | | |
| RAD_kmer_0001211_1_TGCAGGCCAACAGAAGTTCAAGGTT | 230 | AAAACGAGCCATGCACAACTCTAAGATTCTAAATAGTAAGAA | T | T | A | T | GYSNP0357 |
| TGCTTTAACATAGATGTAAATGGTATTCTTCATGTTTCAGC | | ACTTCAAAAGCCACTATGACAAAGTCGGATAGGTCGGT[A/T] | | | | | |
| TGAGGTTATATCCACAGGTAATAAA_NODE_1_length_546_cov_ | | CGAATATTCATTGCAACAAAAATAAATTAAAACCAAAAGTAA | | | | | |
| 73.686813 | | TTTGTAATTCTTTGTGGCAAGATTGAGTTGTTTCCACT | | | | | |
| RAD_kmer_0001211_1_TGCAGGCCAACAGAAGTTCAAGGTT | 333 | TAAATTAAAACCAAAAGTAATTTGTAATTCTTTGTGGCAAGA | M | C | M | A | GYSNP0358 |
| TGCTTTAACATAGATGTAAATGGTATTCTTCATGTTTCAGC | | TTGAGTTGTTTCCACTTGTTCATAACAGCAGCATACAT[A/C]A | | | | | |
| TGAGGTTATATCCACAGGTAATAAA_NODE_1_length_546_cov_ | | AAAATCTGTCGTAACAAGAAATTTTGGAAATAGAAACACCAT | | | | | |
| 73.686813 | | TAATGATCTGATTTTCTTCCTCATGGTCATTGCTCAA | | | | | |
| RAD_kmer_0001215_1_TGCAGGAAGTGGAAAGACAACACT | 251 | TTGTGCTCTTTACAACTATGATATCTAGACCTAAACCATGTTT | A | W | T | W | GYSNP0359 |
| CGCCAAATATATTTATGATTGTAACTTGCTAAAGTTTGAA | | GTATATCTGGAAGCCTACTTGTTATAATAATTTTGCT[T/A]TCT | | | | | |
| AGCAGCAGTTTTCTTGAAGACATTGGA_NODE_1_length_528_ | | ATATTAATCTTTTCCATTCCAAGTAAAGCTTCTATTTGTTCTTC | | | | | |
| cov_131.719696 | | CTTATCAATATCATCAAGAACAATAAGCACCCT | | | | | |
| RAD_kmer_0001218_1_TGCAGGCACAAGTTCCATGATGCAT | 162 | ATCATCCTTTCATTGTTGCTTTCACACTCATCTTTTTCTACAGT | M | C | M | M | GYSNP0360 |
| GCTTATACAAGTGGTTCTTAACTTGTAACAAGAATAGTTG | | CGAAAATTGTGGATTTAACTTCTACCAACTTCAACA[C/A]CCT | | | | | |
| TCCCGTATGCCGATCTCTATTTTGAC_NODE_1_length_337_cov_ | | TCCTTCCTTGAGGTTTTATTTTTATATATGCTGTGCTGTATCTC | | | | | |
| 53.629082 | | TCCTAGCCCTAGGTATATTTATATTATATACTC | | | | | |
| RAD_kmer_0001218_1_TGCAGGCACAAGTTCCATGATGCAT | 266 | TTTATATATGCTGTGCTGTATCTCTCCTAGCCCTAGGTATATT | Y | Y | C | Y | GYSNP0361 |
| GCTTATACAAGTGGTTCTTAACTTGTAACAAGAATAGTTG | | TATATTATATACTCAATTGGGTAAAACTATACATGCT[C/T]AA | | | | | |
| TCCCGTATGCCGATCTCTATTTTGAC_NODE_1_length_337_cov_ | | ATGTAGCATTCTTTATGTTTTGCAAATGTAAACATCTTACTTC | | | | | |
| 53.629082 | | CCCATTGATCATTTTAAACCAACTTCAAATTTATT | | | | | |
| RAD_kmer_0001220_1_TGCAGGACTCGCAGCAGTTTCGGTT | 195 | AAAAATACTAAAATTTTGGGGTTAAAAAGTTGGTATCTCAAC | A | C | C | — | GYSNP0362 |
| TCTAGAATCAAAACAGAAGATATGAGGAGGGCTATAAGA | | TAGAGTTCCAAAATGGTCATCATTTTTTTCCAGAAAAC[C/A]A | | | | | |
| CAAACAGAACCTGTTATTCCCATCATG_NODE_1_length_367_ | | TTAAATTGAATTGGAGTTCAATAACCATCAAAGTTTTCAATC | | | | | |
| cov_51.144413 | | ACTTTAACCATTAATTAGTCTCACAAATTCCCTATCT | | | | | |
| RAD_kmer_0001237_1_TGCAGGGTTTACAAACAGGCAATGT | 150 | TATGATATTGTTAATGAATATTTTTCTTCAATTCTAGGGAATA | G | A | G | — | GYSNP0363 |
| GGACTTTTAATGAATCTAATGCATAACTGATACGGAGTTG | | TCACGTTTTTCCTTTTTTGGTGGAATGCGTAAATAAT[G/A]GAT | | | | | |
| ATCCAAGAGTTAGTGAACTTTTACTT_NODE_2_length_384_ | | TGTCTTATTTTACGTGTTTATTTATGTCTTTAACTTAAATATTT | | | | | |
| cov_28.054688 | | ATTTAACCGTTTTCAAACTCAATGTTTAGGCAA | | | | | |
| RAD_kmer_0001237_1_TGCAGGGTTTACAAACAGGCAATGT | 263 | TTAACTTAAATATTTATTTAACCGTTTTCAAACTCAATGTTTA | T | T | K | — | GYSNP0364 |
| GGACTTTTAATGAATCTAATGCATAACTGATACGGAGTTG | | GGCAATACCTGTAGAAATTACAAACTGTTAAATAAAT[T/G]TT | | | | | |
| ATCCAAGAGTTAGTGAACTTTTACTT_NODE_2_length_384_ | | GAACCTAAAGACATAAATAAACGTATAAATTAAGGCAATTA | | | | | |
| cov_28.054688 | | GTGATATGCCCGTGTTACACTAAAGAGAGGAAAGACT | | | | | |
| RAD_kmer_0001239_1_TGCAGGCAGGAAAATATTTGGGAA | 245 | AAATCGAAGTTGAGGAACTTTATTAGCTTCAGAGAAGTTTTC | G | G | R | R | GYSNP0365 |
| AAAGGTACCCATGTTTGTCTTTCCATCAAATGGGGGAAAC | | AGTTGCAACTTGGATAGTGTCAAAATCAAACCGACGTA[G/A] | | | | | |
| TCAGGTATCATGTTCTCTTCAAGATTA_NODE_2_length_390_ | | TATGTGATTTTCACCTTGAATAGTATCAAGGTGGTACATCAA | | | | | |
| cov_84.505127 | | GGATTTCAAACTTATACTTCCATCTACAAGTTCAACAA | | | | | |
| RAD_kmer_0001243_1_TGCAGGTCTGCACAGATAAATGATG | 133 | AATTGGCATTTTACATATCCGTGGATTAAGGTTCATTTTACAA | R | — | R | G | GYSNP0366 |
| AATGCAAAAATATATAGAAAATAAAACATTATAATATCA | | GGGCACATTGTCGTTTTTTAGTTTATAGAGTAGAACA[G/A]TA | | | | | |
| GTGTCATAAGGCTTACTATAAGACATT_NODE_1_length_589_ | | ATTGTACAATTTCAAGGGTACAATGGTAATTTTACAATTTTAA | | | | | |
| cov_115.899834 | | GGATAAAATGGTCCTTTTACGAAAAAGCAAGAAAT | | | | | |
| RAD_kmer_0001253_1_TGCAGGTAACCCATAAACTATATGT | 109 | TTTCGTGCCATTTCACTCACTCGCTCAAATACAACTTTGTTGT | T | C | Y | C | GYSNP0367 |
| TAAGTGTTATAGTATAGAGTGAGTTAGTTAGCTAGTTTAG | | GATCAAAGTTCTCTCCTTGAAACCTTGGTAGAACCTA[C/T]GA | | | | | |
| TTACTTGTACTTTGTTGATCGTTTGA_NODE_1_length_644_cov_ | | AAGATGAAAAAAGAAAATTTTCAACAAAAGTATATACAGTT | | | | | |
| 100.869568 | | GAGGTTATGTTTATACCAATCAATATGATAACACAAG | | | | | |
| RAD_kmer_0001253_1_TGCAGGTAACCCATAAACTATATGT | 570 | TGGTTGTATTTTGGTTGTTCTTAGATTTAGCATAGATCAAATC | — | Y | T | C | GYSNP0368 |
| TAAGTGTTATAGTATAGAGTGAGTTAGTTAGCTAGTTTAG | | GCTGATCCCGAGGACTAAATTGCTTTCATTTATCCGG[T/C]AA | | | | | |
| TTACTTGTACTTTGTTGATCGTTTGA_NODE_1_length_644_cov_ | | ACATGCATCAAACGATCAACAAAGTACAAGTAACTAAACTA | | | | | |
| 100.869568 | | GCTAACTAACTCACTCTATACTATAACACTTAACATA | | | | | |
| RAD_kmer_0001257_1_TGCAGGTGCAGTCATGTTCTTGTTT | 194 | ATCAATGACCTACATTAAGAGAAACAATTTGACCGTCAAGCA | K | G | G | K | GYSNP0369 |
| GAAGGCGCGTTTGGTAATATTCTGCACACAGGAGACTGCA | | GTGCTGTCAAAACTGCCACGTCGATCAGATTGGGTGCC[G/T]T | | | | | |
| GGCTTACACCTGAGTGTTTGCGCAGA_NODE_1_length_549_ | | GTCTAAGCGGATCAGGCTGAATCCCAAACACTTTTGTCCAAA | | | | | |
| cov_171.843353 | | ATTTCTATTTTTTATGTACCAAAAACATCATTTTTAC | | | | | |
| RAD_kmer_0001268_1_TGCAGGCCAAATCTGATCAGTCTTA | 386 | ACGAGAAACATATAAAAACGCATGTCCGAATAAATAAAAAG | A | A | A | T | GYSNP0370 |
| GCCCAGAATTCTGATTATATGGGCTGCACGCTATTTTCTA | | TCATGGTAAATAATATTTTTAAAACACAAAGTAATTAAA[A/T] | | | | | |
| AAAAAACACTGGGCCGTTGACCAAAT_NODE_1_length_689_ | | ACGATAACCGAAATACTTGTACGCCGAACCGTAGCGTCAAAT | | | | | |
| cov_100.417999 | | CGCGTTCCCCGCGCCTTCTTCTCCGTTTAAACGCGACC | | | | | |
| RAD_kmer_0001268_1_TGCAGGCCAAATCTGATCAGTCTTA | 634 | TTGTTCAGCCTGTAAGTTGTAGGTCGAGACCCCCTTCTTCTTG | A | A | A | C | GYSNP0371 |
| GCCCAGAATTCTGATTATATGGGCTGCACGCTATTTTCTA | | TTAACTCATTCCCCTATTAAATGTTTTCATTTGCTTCA[A/C]CGG | | | | | |
| AAAAAACACTGGGCCGTTGACCAAAT_NODE_1_length_689_ | | CCCAGTGTTTTTTTAGAAAATAGCGTGCAGCCCATATAATCA | | | | | |
| cov_100.417999 | | GAATTCTGGGCTAAGACTGATCAGATTTGGCCTGC | | | | | |
| RAD_kmer_0001271_1_TGCAGGTCTTTTCATTTCTTGAGTAT | 407 | GAATTTCCAAACTTGGACAAGCTCTAACTATTGAAGTGAGGC | A | — | R | G | GYSNP0372 |
| TATCCCCAGGTTCGCAATTCATCCCTAGTATATTTATAGCC | | TTAGTAAACCTTCATCTTCATGAGCTCCGATAATCAGC[A/G]T | | | | | |
| ATTAATAGATATATTGTGAAATTG_NODE_1_length_541_cov_ | | CAGTTCCTTGACATTTGATAAAATAGGTAGGGAATTGAGCAC | | | | | |
| 106.574860 | | CAACAATCGCTACAACAAGAAAGAAACAATTTCACAA | | | | | |
| RAD_kmer_0001271_1_TGCAGGTCTTTTCATTTCTTGAGTAT | 484 | AGCATCAGTTCCTTGACATTTGATAAAATAGGTAGGGAATTG | G | A | A | A | GYSNP0373 |
| TATCCCCAGGTTCGCAATTCATCCCTAGTATATTTATAGCC | | AGCACCAACAATCGCTACAACAAGAAAGAAACAATTTC[A/G] | | | | | |
| ATTAATAGATATATTGTGAAATTG_NODE_1_length_541_cov_ | | CAATATATCTATTAATGGCTATAAATATACTAGGGATGAATT | | | | | |
| 106.574860 | | GCGAACCTGGGGATAATACTCAAGAAATGAAAAGACCT | | | | | |
| RAD_kmer_0001277_1_TGCAGGATCCTTTTCGTTTTAAATAT | 133 | TTACTTCGTTTCCTAGAATAGGTCCCCTTGTATCATTATATAT | T | — | W | T | GYSNP0374 |
| AGGCAGCCGTACTAAAAAGAAAATGTAACCTTAAATGTG | | TGTAACATTATTTGTGAATAATAATTAATTGATTGAG[A/T]TC | | | | | |
| GAAAAAAGGAAAGAAAGGTTACCGTC_NODE_1_length_493_ | | TCGTAATATATGATCCTAAACATGTGATATAAGTTTTATCGTG | | | | | |
| cov_93.945236 | | TTATTTTCTGTAGATCTAAAAATCTAAAGTAAAAA | | | | | |
| RAD_kmer_0001283_1_TGCAGGAAGAATCGCGGCAATCAT | 161 | GACCCATTCATAAGTAAACGTGCCTAAACTTCCAATTTTTCAT | G | A | G | R | GYSNP0375 |
| AGGCAGATCGCCCAAAAAGTTCAAATGGTACAGAAAAGA | | CTGGGCGTTTTATCCATAGGTTTCTATGTAGAGAAAT[G/A]TA | | | | | |
| ACTCGTTATATATAAGCCCTGTGTAGAT_NODE_1_length_485_ | | TGCAACCATGACCCAACTTTGTCTTTCTCCCTTCTCTGTCTAT | | | | | |
| cov_163.936081 | | AAAAGGGGCAATGGTTTTAATGTAAAATCTAAAGT | | | | | |
| RAD_kmer_0001289_1_TGCAGGATCTGCGCATGCGCATAAT | 488 | AGCAAATACGGCGCTGTTCTTAACTTTACTTGCATTGAGATG | T | T | G | T | GYSNP0376 |
| CATCATATCTAGGCAATGCATTCTCTCCAGCGATCAACAC | | AGGAACCATGAGCAGCCTCAGGAGGTGCATTGTGCTCC[G/T] | | | | | |
| ATGTGCTTCTCGTGTCGCCAACGCCA_NODE_1_length_572_ | | GAGAATCTTGTGCAGCAAGTGGCGTTGGCGACACGAGAAGC | | | | | |
| cov_135.288467 | | ACATGTGTTGATCGCTGGAGAGAATGCATTGCCTAGATA | | | | | |
| RAD_kmer_0001301_1_TGCAGGTTTACTATAAAGCAACTAA | 465 | GATATTAATCAAGATTACTTTCCACATCGGGTTTGCAGGCAA | — | T | A | T | GYSNP0377 |
| TTTGCCCACATCTTCCTGCGTCGTCTTTTTGGTGGAAGCGG | | TTTGGGTATGCCCAAGATATTCTAGCAGATATCATACT[A/T]G | | | | | |
| GACCCGTGGGGCCAAAGGATCCATG_NODE_1_length_658_ | | TTGACGACAATGAAGATGCTTGGGTTGACTATTCTACACCGC | | | | | |
| cov_67.209724 | | TGTTGAATCTAGAGGTATACATTCCACCAATAAAGTT | | | | | |
| RAD_kmer_0001305_1_TGCAGGACTAAAGTCAGGAGGTAT | 373 | TATTAAATATAATAATTTTATATATTTAAAGTCATCTCACATT | R | R | R | R | GYSNP0378 |
| ATAGGATAACAATTATGAATTTTACTTAATTGGAGTTCTA | | GTTACTTAAGTCACTAACTAATAATTTAATATATTAT[A/G]TTT | | | | | |
| AATTCAAGAAAAGAAAATCTATACCTA_NODE_1_length_545_ | | TACTACTATTTGTTTTCAATTAACTTTTTCAAATTAAGAAATA | | | | | |
| cov_72.849541 | | AAATATGAAGGTGGTGAATAAGAGAAGGAAGAAA | | | | | |
| RAD_kmer_0001308_1_TGCAGGATGACATTAAATTTACACA | 463 | TCTGTTTATAAGCGGAAGTAGAAGATTAACTTACTGAAAATA | A | A | C | — | GYSNP0379 |
| CTTTATCATACTCTTCTATTAATAGGTTACCCACCCACCCA | | CCTTATTTGGGTATAACTTATTAATATATTATAGAAAC[C/A]T | | | | | |
| TTTATAAAACATCAAAGTTTACAAA_NODE_1_length_599_cov_ | | CTACTTGAGATCTGCTTATTGTAACAACTTATTTCATCCTAAA | | | | | |
| 72.642738 | | ATAAGCATACACACCATATATTCTTTTTTTGTAAAC | | | | | |
| RAD_kmer_0001310_1_TGCAGGCTGCAAATCATTAGCCAAC | 123 | AAACAAGAAAAAGAAGAATAGTTGATTTATTTGAAGTTATGA | T | C | C | C | GYSNP0380 |
| AAAACCATATGGAACTACATCAACTTCTGAACAAAGATTC | | TGTCTTAGTGGCGCATTCATTGTATCTTTTATGTTGTT[C/T]AA | | | | | |
| ATAAATTTTAATAGCGATTATTAGAC_NODE_1_length_606_ | | GAATTTGCTGGGTTAGTTTGATAACATAAGGGGGAGAAGGTG | | | | | |
| cov_81.768974 | | TTAGGATCGGGTATCACGGCTGTTCAAATCATTTGA | | | | | |
| RAD_kmer_0001313_1_TGCAGGCCATCCTTGGGTACATGTT | 310 | TTTCCGACTCCTTAAGGTTAGCACCAACTCGTTTTAATCCAGC | K | G | K | G | GYSNP0381 |
| GACGGAGTGGCTCCAGATAAGCCGCTTGACTCTGCGGTTC | | CTTGAGCTGATCAAAAGTAATGTGACCACTATTATCG[G/T]TA | | | | | |
| TGAGTCGCATGAAACAGTTTTCAGCC_NODE_1_length_550_ | | TCTATCATCTGAAACATTTGTTTCAGCCCAGCTATTTCTTCTTC | | | | | |
| cov_93.465454 | | TGATAGGCTCTCTGCTATAACCTGCATCAAACTT | | | | | |
| RAD_kmer_0001315_1_TGCAGGGATATGCCCGAGTGTTGGC | 130 | TTTTGCCAATGATGGATGAGATCGATGGTGTGTTGATCCATGT | K | T | K | T | GYSNP0382 |
| GTTGGTGGGCACTTCAGTGGTGGCGGCTTCGGTGCTATGG | | TCTTGTAGACGTTGAAAATTGTGACTTTTGGTGGAAC[G/T]CT | | | | | |
| TGAGGAAATACGGCCTAGCGGCAGAT_NODE_1_length_338_ | | AACGAGCTTGATTTTCCATGAGACTATAACACCAAAGCTCGC | | | | | |
| cov_61.772190 | | CCCACCGCCACCTCTAATGGCCCAAAAAAGATCTTC | | | | | |
| RAD_kmer_0001319_1_TGCAGGTACCATAATCTTTTCTTTTA | 344 | AATCACTTCGTTTTTTTTTAGATTTTCGGTAAAATACTTCTTTT | R | A | R | A | GYSNP0383 |
| TAGAGGTGGTAGAACCGGGTTGAGTAACAAGTATTTTGTA | | TTACAGGAATTTATTATACAAAATTTGGACATGCAT[G/A]GAA | | | | | |
| TAGACAGGGGCGTACCCACATGTAA_NODE_1_length_546_ | | GTTTTAGGAACCCAATTCACCCTGGCCCATCATCGGCCAATC | | | | | |
| cov_137.333328 | | GGGGAATTAACCAAACTTCTAAGGATTTAATAAAA | | | | | |
| RAD_kmer_0001321_1_TGCAGGGATGTAGAAAAGTTGCAC | 172 | GTAACAAAGCTTCCTGGGGACGGAGTTTATTCAGGCTGTACA | W | T | W | T | GYSNP0384 |
| ATTATTAGGTCCTTCTCACAAGAAAAGCACATGAAACACC | | TGCAAACAGGGTGAGATTGAAGCAGTAGTCATCGCCAC[T/A] | | | | | |
| AAAAGCACAAAGAAATTTTAACCAACC_NODE_1_length_642_ | | GGTGTGCATACCTTCTTTGGAAAGGCTGCACATCTTGTAGATT | | | | | |
| cov_119.445480 | | CTACAAACCAAGTAGGCCATTTTCAGAAGGTATGAAA | | | | | |
| RAD_kmer_0001322_1_TGCAGGATTTTGGTCCGAAATGGCT | 222 | TCCTTCCCAGTGAATCGCGATCTTCTCACCGTTTCCAGCTTCT | T | T | C | T | GYSNP0385 |
| GCCGACTTCTACTGGAAGAAGAAATGGGGTCAACAGGTC | | ATGTTTCTATCCAAACAGTTATAGCAGATGTTTGTGA[C/T]TC | | | | | |
| TACTATGAGAATCTTGATATTACAAAA_NODE_1_length_395_ | | CACCCTTGAACCACTGGCAAGCACACCAACTTGTTAAGGATG | | | | | |
| cov_103.584808 | | GTATACAGTTGCAAGTGAAAAACATTAACAGTTGAT | | | | | |
| RAD_kmer_0001322_1_TGCAGGATTTTGGTCCGAAATGGCT | 320 | GGCAAGCACACCAACTTGTTAAGGATGGTATACAGTTGCAAG | T | T | C | T | GYSNP0386 |
| GCCGACTTCTACTGGAAGAAGAAATGGGGTCAACAGGTC | | TGAAAAAGATTAACAGTTGATTACTGATTACCTCTATA[C/T]T | | | | | |
| TACTATGAGAATCTTGATATTACAAAA_NODE_1_length_395_ | | AATATTTCCTTTTGTAATATCAAGATTCTCATAGTAGACCTGT | | | | | |
| cov_103.584808 | | TGACCCCATTTCTTCTTCCAGTAGAAGTCGGCAGCC | | | | | |
| RAD_kmer_0001323_1_TGCAGGCTGCAAATCATTAGCCAGC | 385 | GAACGTCTAAACTGACCTGATTACAGGATGGATGGATGTTTT | R | — | G | G | GYSNP0387 |
| AAAACCATATGGAACTACATCAACTTCTGAACAAAGATTT | | TACTGCTAGTCATGCTTGATTACAGGTAGAGGACTTAG[G/A]A | | | | | |
| ATAAAATTTTAATAGCGATTATTAGA_NODE_1_length_519_ | | AAGTTGCATTCATATATGGGGAGGAATAGGTATTAGTAGGTT | | | | | |
| cov_91.067436 | | TTTAAATTATTTTAGGTTGTATCCTGTCTAATAATCG | | | | | |
| RAD_kmer_0001326_1_TGCAGGTGTTTATCGAGCATTTGCA | 362 | AACACTATATAATTTTGTTTTCCAATTTTCAAACCGAAAACAT | Y | T | C | T | GYSNP0388 |
| AGAACTGGCGGTTCAAGGTTGATTAAATGTCGGTTTTGTC | | CCGGTTCTGATTAAAACCGAACCGAATGGAACCAAAC[C/T]G | | | | | |
| ATCGTTGTTGTTGTTGTAGTATAGAT_NODE_1_length_493_ | | TAATACACAATAACTTCTTCCTGTAGATGATCTGCTCGTAAAA | | | | | |
| cov_119.486816 | | CCCGACGCTAGAGAGGAACCGTATCTATACTACAAC | | | | | |
| RAD_kmer_0001329_1_TGCAGGTTATTTGAAGCACCAATAT | 118 | ACTATAATAAGTTACTTAGATAAAGGATGAAATGTTATATAT | T | — | T | K | GYSNP0389 |
| CACACGTACAAACCTAAAAAAGATAAAAGATATCTAAAC | | TGATTGTGGTGTTGCTAATTAGCGTTACAAGGGTGTCC[T/G]T | | | | | |
| AAGTTTAACTCTCATTAATTTTGTATT_NODE_1_length_590_ | | GTATATAAAGAGAACTGGCTTACAATCTCGTGTATTATACGG | | | | | |
| cov_94.132202 | | GCTGGTAAATGTAATGTTCTAAATAAAAAATTTATGA | | | | | |
| RAD_kmer_0001330_1_TGCAGGATTGCAAACACAATATTGA | 106 | AAGTATATACTTCTTAGGTGGTTCATTATTTCCACTCATGATT | G | S | S | G | GYSNP0390 |
| AGATAACCATAACATTTAACAAACTCACATACCAAGAAA | | GTAATTTTGAATCACATGATATTATTCTGACAACAA[G/C]GT | | | | | |
| ATAACCATTGTAACAATATTGGTGAAA_NODE_5_length_262_ | | CACTTAGCAACTTATGTTCTATCCGAGAGTATATGTTTTTAAA | | | | | |
| cov_37.744274 | | TATCTTTGTCAGTAAATGCATCTTTTGTAAAATTC | | | | | |
| RAD_kmer_0001333_1_TGCAGGATGCAGAAACCCGGTTGAC | 244 | GCAGTAATTGTAGGGAGTGATATGAGTGTGCAGACAAAATTT | T | C | C | C | GYSNP0391 |
| CCGTTCACCACAATCTCTGCAGTCCCTCCTGCTGATGTTCC | | GAAACAGAACTTAGGGATTTTGTAGCACAGAAAAAGAT[C/T] | | | | | |
| CTGCCAATTAACAAACATATATGTC_NODE_1_length_606_cov_ | | CAACATCGTGTTCATTTCGTTAATAAAACTCTTACTGTAGCCC | | | | | |
| 154.839935 | | CGTATTTAGCAGCAATTGATGTCCTAGTCCAAAACTC | | | | | |
| RAD_kmer_0001336_1_TGCAGGGTGCAACTTCATTCAAACA | 108 | CTATTTATAGTCATCCTCACCACAGTTGTAATACACCTGTC | C | M | M | M | GYSNP0392 |
| GGTTAATTTTAATCAAATAGTTCTCTAGAACTTGAATCTAT | | AAATAAAGATAAGTACTATTTATGTAAAAACGTAAAA[A/C]T | | | | | |
| TAAACCCTAATTACAAGATTAATTA_NODE_1_length_505_cov_ | | ACAAGGACGGAAATGACACTTAAGAAATAATACAGGGTCAT | | | | | |
| 153.463364 | | TTATTGACAAACTTTGATTCATACATGCATAAAACCTT | | | | | |
| RAD_kmer_0001336_1_TGCAGGGTGCAACTTCATTCAAACA | 219 | ATACAGGGTCATTTATTGACAAACTTTGATTCATACATGCATA | R | G | R | G | GYSNP0393 |
| GGTTAATTTTAATCAAATAGTTCTCTAGAACTTGAATCTAT | | AAACCTTCATAAATTCATCAAGCCAAATCACAAGGTG[G/A]C | | | | | |
| TAAACCCTAATTACAAGATTAATTA_NODE_1_length_505_cov_ | | ATTAAGCTTCACACAAATTGTAACGTCGATTGCATCAGATTA | | | | | |
| 153.463364 | | TCCCTTTCTTTCCTCTTTTGCTTCTTCTCTCTCTAAA | | | | | |
| RAD_kmer_0001338_1_TGCAGGAATGAAGGTTCTGTATTTT | 157 | TTTTGTTTCCATGTTGAGAATATGGCATGTAAAAGCAGCAAA | A | A | T | A | GYSNP0394 |
| TTTCTCACATTTAGTTGTGAACATGCTTGATGATTCGAGCT | | ATATGTCATTGTATAGGAGTTTAAAACAATCTATTGTC[T/A]A | | | | | |
| ATATATAAGAGTTTAGTTTGTTGCC_NODE_1_length_645_cov_ | | CATGACAAAACAGTTATCAGCTGCTCGAAATACAAAAAAGAT | | | | | |
| 118.851166 | | TCTTATATACTCCCTTTTTTGTGGTTTATGAAAAC | | | | | |
| RAD_kmer_0001342_1_TGCAGGCAACGGAAGGCGGCCA | 229 | CAACAATAATAAGTTATTGTGTCCACTAAACGAGCAACATCA | A | W | T | W | GYSNP0395 |
| TGGTGGCAAACATGGAGAGGATGAGAGATGAGAGTCTCC | | GTTTGTTGTTCCTCCATCTCTTTCTTTATCCCACCAAC[T/A]GC | | | | | |
| ATTGGAACGCTGAACGAGAGGATAGCCG_NODE_1_length_ | | CACACAACACAAGTGTGCTTTACATTCCTCCCAAATCGACGG | | | | | |
| 470_cov_126.774467 | | ACACAACAAATCTAACCAACTCTCATTACGATCTCA | | | | | |
| RAD_kmer_0001343_1_TGCAGGTCTATAGCTAGGGTAGAAA | 399 | CCCTCCGGTGACCAAGTTTGCCGTTAACGGTGGTCTATATTTT | T | A | T | T | GYSNP0396 |
| GCAACATAGTTTCAACTAGTCAACCACTCATATATAACCT | | GCCACTTTGTTTTTGTTGTTTGATCCTTCTTAAACTC[T/A]CAA | | | | | |
| CAACAAAAGTTTAATAAAAAGTTAAA_NODE_1_length_632_ | | ATCTCACCTGTAACGTAGTTAAAGCAAAGTGAGAGTTGTAGT | | | | | |
| cov_84.221519 | | AACATCATCTATTGCTTTTTTCCGGCAAAAAAGGA | | | | | |
| RAD_kmer_0001345_1_TGCAGG GTTTATCTAATGGACAA | 241 | AATAATCAAATTTATCACCCATTTTTAAGCAATAAAAAAGAT | G | A | R | A | GYSNP0397 |
| TATGCAATGAATAATCTTCCTATTACACGTTCTTTGGTATT | | TATACAGAAAGATAGTGCGAATGTATTATCCAAAAGCT[A/G] | | | | | |
| TTGTTTAGAAACATATTTTTCAATT_NODE_1_length_623_cov_ | | TAATACCTAGTTATCTTTTAGAACCACCAGCAGAGCTTAGAT | | | | | |
| 153.988770 | | GTCAATTTAACTTGGGATATTATTCATGTCAAACTAAT | | | | | |
| RAD_kmer_0001346_1_TGCAGGCATACAGGGAGCCAGGCA | 477 | TACTTTGTCTTCATTTTGGTCCAATTTATAGCTAGGATTACCA | K | K | K | K | GYSNP0398 |
| TGCGCGATTTCAGATCCCATTTTCGGATCTTAACCTAAAC | | ATGGTCAGCAAGCATGATATGTCATCATTATTGCCAT[T/G]AA | | | | | |
| CACAAACTTCACTTAAACCTTTTCCCT_NODE_1_length_581_ | | TGACATCATCAGGACCGTTGGGTGAATCAAAGTGTTTAGGGA | | | | | |
| cov_104.275391 | | AAAGGTTTAAGTGAAGTTTGTGGTTTAGGTTAAGAT | | | | | |
| RAD_kmer_0001347_1_TGCAGGAAAATGGCGCCAAATTTAG | 112 | AACAGGTCAAATAGGTGAAATATAAATATAATTAGCTTATAA | T | A | A | W | GYSNP0399 |
| AACAGTTGGTTATGTAAAGCACTAAAGCAGTTATTTAACC | | GGGATCCGGTCAAATGGGTTCAAAGTTACCCAGAATTT[A/T]T | | | | | |
| GCCAATATCAAAGGCTATATAAGGCC_NODE_1_length_590_ | | TTTTTTTAATTCATAAAACCTGAATTATTTTAGAAAAAAATAG | | | | | |
| cov_101.198303 | | ATTATTACTGTAACAGTATAATCTTTATAATCATAT | | | | | |
| RAD_kmer_0001351_1_TGCAGGCGGTTGAGGATAGAGATC | 510 | AAAATATCAAAACCAAAGACCGATGATGTCCCATCATTGGTT | A | R | R | A | GYSNP0400 |
| ACCCCCACCGATACCAGGAACCACCGGAGGCATAACCAT | | TTTCTTAATTCAACTTCAAGTTGCATCTTCCATGTAAC[A/G]AC | | | | | |
| CCTGCATTTTAGTTTCCTAATTAGCCAA_NODE_1_length_604_ | | ATGAGATTGAGATTGTAATATATCAGTTTGGCTAATTAGGAA | | | | | |
| cov_123.867546 | | ACTAAAATGCAGGATGGTTATGCCTCCGGTGGTTCC | | | | | |
| RAD_kmer_0001353_1_TGCAGGAAAGAAAGGGGAATGAGA | 330 | CAGATGCTCAACCATTTCTGTAGGTAGCAAAGGATTTGTCTC | A | T | A | T | GYSNP0401 |
| AAATCAAATCATCTTTGGTGAACGATCTTTTGCTGCATGT | | CTGTAAGGAAATACATTAGTGAAACAGAAACCAATAAA[A/T] | | | | | |
| AAATGAGAAATCATAAAGAAACTAA_NODE_1_length_514_ | | ATAATAATAAAAAGACTTGAAAACAAAGAAAAATGATGGTA | | | | | |
| cov_137.922180 | | TCTACATTACAAAGTGCTTCGGGTGAAAAAGCACTCGAG | | | | | |
| RAD_kmer_0001355_1_TGCAGGTTTTGGTTTCCAGATATAA | 164 | GCTTGTAAAAATAAAAACTATAAAGGTTGGTTTTAATGTCCG | G | R | G | R | GYSNP0402 |
| GAACTAGCTAAGAAAATGTGCACTTTGACTTTTTTGTC | | CTGGGTTTAGTTACTTTATACACATATAATTATAACCA[G/A]T | | | | | |
| AAACTGACTGATATGAAACTTGTTAG_NODE_1_length_684_ | | GTTGTAGAAATCGCTTGACGCTAGTTGGGCGTGAGGTACCAC | | | | | |
| cov_62.687134 | | CTAGGCTTAATCGGAATTAGTCGGGCGGTGAGGTACC | | | | | |
| RAD_kmer_0001355_1_TGCAGGTTTTGGTTTCCAGATATAA | 292 | GCTTAATCGGAATTAGTCGGGCGGTGAGGTACCGCCTAGGCC | G | R | G | R | GYSNP0403 |
| GAACTAGCTAAGAAAATGTGCACTTTGACTTTTTTAAGTC | | GATTTCTACAACACTGATTATAACAGAATCATAAAGTC[G/A]C | | | | | |
| AAACTGACTGATATGAAACTTGTTAG_NODE_1_length_684_ | | TAATCAAAAAATTTATAACCGAATCGTTGGTTTCTCAATTAGT | | | | | |
| cov.62.687134 | | TCGTTATGTTGCTTATCTGATTTGTCTACTAACTCC | | | | | |
| RAD_kmer_0001358_1_TGCAGGGACAAATGGGGGATCACTT | 252 | ATATGTACCGAATTAGGAAGGAGATATATGAATTATGAAGTT | S | S | C | S | GYSNP0404 |
| TTTCTCAGCACATGAAAACAATCAACCTAGAAGCAACATC | | ATCTTTATTATAATGTATGCAACTCCTTTTACTTAATT[C/G]GA | | | | | |
| AAAGTTACACTTATATAACAATTCTG_NODE_1_length_514_ | | CTGTTTGTATATTAGGTACCGTGTCTCTCTATTTGCCAGATGG | | | | | |
| cov_153.570038 | | AAACAAAGGGGGATGTATGCAGCATGTATGTGGAT | | | | | |
| RAD_kmer_0001361_1_TGCAGGAAGCGTAAATTCATCTATG | 410 | AATTCCCTCCAAGTTTTGGCGGTGGTGGAGGTGGCGGTGAAG | G | A | A | — | GYSNP0405 |
| CAACATACTCTTAAGCCTAGAGTTGTGAGGGCAGAATCAT | | GCGCTACTTCACGCAGTGCCTCATCAGAAGAAGGTCCA[A/G] | | | | | |
| CTTTGCGGCCTACCAGAAAGACAGAA_NODE_1_length_635_ | | GTGTAATGGGAGCAGAACCAGAGGCTTCCGGAGGAGAAGCT | | | | | |
| cov_90.703934 | | GGAGGAGCAGTGGCGCCAGAAGAAGCCAGAGAATCTCCA | | | | | |
| RAD_kmer_0001369_1_TGCAGGGAATCCAAACATGTACCAG | 501 | TTTAAACTATCAAATTAAAGTAGCATGTTTATGTGTTGTCGAA | C | — | A | C | GYSNP0406 |
| GTATGTTTCTTTATTATAATGTTTACCAATTTTCTTAATCCT | | CAGTGGTCTATTCAACTGTCTTAACTTTTTAATCAGA[A/C]CA | | | | | |
| GATTTTCCACACGTAATGTATATT_NODE_1_length_617_cov_ | | CACATTCAAGCATATGACAGGTAATATATTGTAACATCTCTA | | | | | |
| 75.437599 | | TAACAGAAATATACATTACGTGTGGAAAATCAGGAT | | | | | |
| RAD_kmer_0001371_1_TGCAGGTTACAGTAGTCACTACACG | 278 | TATGGGGTGGTTACGCGGATTGTTGTTGGTGGGTTTTTGGTTG | K | T | K | T | GYSNP0407 |
| ACAAAACACATCCAAACACCCCATAACTTTTATTGATTAC | | GGGTTCTTGTGATTGTGAATTTGGTTGGGTTTTTGGT[G/T]CAG | | | | | |
| GCAATAATAAAAGTATCCAAATTTAC_NODE_1_length_407_ | | AGTGTGTTTTATTATGTTTGTAAGAGTTTTCATCATCAAGGGA | | | | | |
| cov_52.761669 | | TTGATAAGAACGCGTTGTATGATCATTTGGGTGG | | | | | |
| RAD_kmer_0001375_1_TGCAGGACCCGACAACACCGCTTTA | 203 | GATTGGTAACCAGGCCCGTCCGAATATTTTTGATCTCACTGTA | C | Y | Y | T | GYSNP0408 |
| TGGCCCGAGAATCTGCTTTCGGGAGCAAGACCGCCATCCG | | TCAAAACCGTCTAATCTTTATGAAGAGGTTATAGAAG[T/C]TG | | | | | |
| ATTGCATAAAAAAAGAACATTAACCTTT_NODE_1_length_661_ | | ATGAGAGAATTGAGCTTGTTCCTGAGTCCGAGAAAACGATTT | | | | | |
| cov_115.547653 | | CCAACTCTGGTGACTTGAATGTGGTTACTGGCGTTT | | | | | |
| RAD_kmer_0001375_1_TGCAGGACCCGACAACACCGCTTTA | 384 | GTGAAGCCTGTTAGTGAAGAAACGTTGAGGCCGTTACTTGCT | S | S | S | S | GYSNP0409 |
| TGGCCCGAGAATCTGCTTTCGGGAGCAAGACCGCCATCCG | | GGTTTGTTAGAGAAAGGGATTAAGTGTTTAGCAGTTGT[C/G]T | | | | | |
| ATTGCATAAAAAGAACATTAACCTTT_NODE_1_length_661_ | | TAATGCATTCTTATACATATCCGCACCATGAGCTTTTTGTGAA | | | | | |
| cov_115.547653 | | CAATTTGGCTGTGAGCATGGGGTTCAAGCATGTTTC | | | | | |
| RAD_kmer_0001376_1_TGCAGGAATCAACAAGCATCACCCT | 498 | ACTTGTACAAAACTGATTATATGATCCCTAGATTAGTCAGAC | C | T | T | — | GYSNP0410 |
| AAGTATTTATATATAATCCAATCATTTTAAGAGGTGTGTG | | AATCTGAATCATTTTATATTAAACAATATCTACAACTT[T/C]A | | | | | |
| GCAATGCTTTTTGGAAGTGATTATCT_NODE_1_length_577_ | | AAACAGTAGAAGCAGATAATCACTTCCAAAAAGCATTGCCAC | | | | | |
| cov_79.727905 | | ACACCTCTTAAAATGATTGGATTATATATAAATACTT | | | | | |
| RAD_kmer_0001383_1_TGCAGGTTCATTCAGCAAATGTAAC | 112 | TCCTCGCCCTGCAGTAAAATTGCAGATGGCAATTTTGACCAG | A | G | R | R | GYSNP0411 |
| ACTTTTGCTTGTAATCCTTTTTTTCTTTTTGAAAATAATTA | | TTTACGATTGAAAGGATAGATTTAGTTGTGTTTGATTT[A/G]A | | | | | |
| AATTGGGTTATATTTAACTGATAA_NODE_1_length_621_cov_ | | GCATGTCACATAAAAAAAGTTGGCTAAAGTGGGTACGAAAA | | | | | |
| 84.165863 | | AATTAGATAAAGAAGGTACAGATCAAATGGGTTGAAAT | | | | | |
| RAD_kmer_0001384_1_TGCAGGGTTACAAATTAATACTTTT | 354 | AATGGTGAGAAAAATAGCAGGAAAGAGTATAATGGAGGATT | R | G | G | R | GYSNP0412 |
| AAGTTCCAAGACCATCCAAATGAAGATGCCAAAACAAAG | | CAGAAAGTGAGACCATTGTTGCTTTAGAGGGCATAATAT[G/A] | | | | | |
| TTAAACAAGTTACAATCATGAATACTG_NODE_1_length_615_ | | TCATCTTTGGTCATGTTTCCACAATAGATATTGAAATTAATTT | | | | | |
| cov_113.160973 | | GTGACCATGAAAAGAATGCTAGTTTGATTTTCTGCTG | | | | | |
| RAD_kmer_0001400_1_TGCAGGTAAAGGAGATATTGTCTCA | 82 | AAAGACCTTAGATTGTAAAATTTACCGACAAATATTCTATGT | G | — | R | G | GYSNP0413 |
| TTATCCGTCAAATTACAAGCAATCAGCAGTCATTCCTCTG | | CGATTCACTGAATAAAATACTTGCGATATATTTTATAT[A/G]T | | | | | |
| CTGGATCTTGCACAACAGCAGCATGG_NODE_1_length_502_ | | AACCCTTAGATTATTTAATAATCCTTAATTACAATGTAAATGC | | | | | |
| cov.115.017929 | | | | | | | |
| RAD_kmer_0001403_1_TGCAGGAGGTAATTGGGAATTTGGG | 84 | ATGCAATAAACACTATTTTTTTGGTATTATAAAATGCAAATGT | Y | T | T | Y | GYSNP0414 |
| ATTAAGCATGATGGAATGATTTAATATGAGAAAAATCAA | | TTTGCTTGGAGAAAATCAAGATAACTTAATTTTTTTG[T/C]TGT | | | | | |
| ATGGAAGATCCATACCGCACGAGACTG_NODE_1_length_538_ | | TTCTCAGGTGGTCCATACTCAAGCAAAGGTGATTGCAGATGA | | | | | |
| cov_137.618958 | | GGATGTGATGTATAAATACATATCGAAAAATATGC | | | | | |
| RAD_kmer_0001404_1_TGCAGGTGAATAATCAAGATTAGTGA | 383 | TACTTGAGTTTAATATTCTTTTTGTTTGCTTCACAGGTTTATGT | Y | Y | C | Y | GYSNP0415 |
| TATCCAAACTCTTCTAATCAAGTCCATTACAGAACTTCAC | | ATGGTGCAAGCAAAATCCATTAATTCATATGAGTTT[C/T]CTG | | | | | |
| ATTCTTAATATGGATGCAATGTATAA_NODE_1_length_524_ | | GGCATCTTTACTTTTACAGCAGGTTACTTACCATGGGTGAGTT | | | | | |
| cov_59.769085 | | CTTGTCAATACATATATCTATAACTTCTAAAAAT | | | | | |
| RAD_kmer_0001407_1_TGCAGGCTGTCCTTTGTCTCCGTTCT | 438 | ATACTATTCAAAGTCTACACCATAAAACTATTCATGCCTAGT | R | G | G | A | GYSNP0416 |
| GATACTGGCACACTAAACACTAGACATGAATAGTTTTCGC | | GTTTACTGTGTGGGATCAGAAACGGTCAAATGGAGACA[G/A] | | | | | |
| TGAGTTGACTTTGAATAGTTACTGA_NODE_1_length_509_cov_ | | GACAGCCTGCGGGAGGAGCCGAGAATGAAGCGTTTGGATAT | | | | | |
| 52.799606 | | CAGGTCTTGAGCAAATGACACTAGAAATAAGTCAGCTGA | | | | | |
| RAD_kmer_0001408_1_TGCAGGGAAAGAGAAGCACAATCT | 226 | ATTATCAGGATGGACACACAGGGACACCTTTCTGTAAGCTTT | C | C | T | — | GYSNP0417 |
| AATTGCTCTTAGAGAACAAGTCGAGAGAAATGTAATCATC | | CTTAACCACAGTGGTGATTATGATCTCTGTCAGTGACA[T/C]A | | | | | |
| TTTTGAATCAAATTTTCCGTTTTACCA_NODE_2_length_490_ | | GCTTACCAACAACTGGCGGCTCCAAGAATCTGTAGCAAATAT | | | | | |
| cov.52.179592 | | AAACATTCGAGCTAAATTTATCTTATATCCAGGTATG | | | | | |
| RAD_kmer_0001408_1_TGCAGGGAAAGAGAAGCACAATCT | 361 | CTAAATTTATCTTATATCCAGGTATGGTATAAAATTAGCAAA | C | C | G | C | GYSNP0418 |
| AATTGCTCTTAGAGAACAAGTCGAGAGAAATGTAATCATC | | AGATTACGTTAATGGGTGCCCCTCAAAGATAGAATCCA[G/C] | | | | | |
| TTTTGAATCAAATTTTCCGTTTTACCA_NODE_2_length_490_ | | GGGTGTATAATATTTAAGAAGATTTAGAAGGTTATACATACA | | | | | |
| cov_52.179592 | | TGTTGCAGCGTTGAAAGCAATGCACGCAAGTTCCCTCC | | | | | |
| RAD_kmer_0001410_1_TGCAGGGTAATACTAGGTGTTGTAA | 177 | AGGATTACACACTTACAAGTTACACCTAGACAGGTTTCTCAT | T | — | T | C | GYSNP0419 |
| AGAGGGGATTCCTTTGTATTGGATTGCCCCAAGGTGTTAG | | ACCCACCAAAAAGATTCATTAATCAGAAGTCCACGAAC[T/C] | | | | | |
| TTGTGTAGATATTGTTATCTTTCTTT_NODE_1_length_609_cov_ | | GCCACATCATCCTTGCCTAGCGAAGCACCCCACGGCCCAACC | | | | | |
| 103.036125 | | TCCTCTCTCACAAACCTGCAAGTAGAGCTGACATGGCC | | | | | |
| RAD_kmer_0001410_1_TGCAGGGTAATACTAGGTGTTGT | 403 | CGCGTGGGTCGATAGAACCCCTTGCAGATGCTCTAATCTTTGT | T | — | T | K | GYSNP0420 |
| AGAGGGGATTCCTTTGTATTGGATTGCCCCAAGGTGTTAG | | TTCCATCATGTTTATATCATCGGTTCATGTGATGCAT[T/G]TTT | | | | | |
| TTGTGTAGATATTGTTATCTTTCTTT_NODE_1_length_609_cov_ | | TTAATAAAACTGGAAAATGAACATATAAAGATTTTACACAGC | | | | | |
| 103.036125 | | TAGAGCATCTCAATGCATATTTATTTGACGTGTAT | | | | | |
| RAD_kmer_0001424_1_TGCAGGAAATGCGAAATTTGTTTTT | 290 | GGGGTTGACATGCTAACATGTGATTGGTTGCCCGGACGTTCC | K | G | K | T | GYSNP0421 |
| CTTAATCAACTTTAGGTTCCTTTGAATTTTAGATTAGTTCT | | GTTGGCACTTTATTAGCTTCTTTCCCTTATTTTTCCTA[T/G]ATT | | | | | |
| TGTTAATCAACTTCTGAGTTTGAT_NODE_1_length_471_cov_ | | CATTCTGCTCCATTGGCGCTTATTTAACACTAACCACGTCATT | | | | | |
| 102.513802 | | GGCATGTTTTTTCCATAACATTATTTTTTTTTAA | | | | | |
| RAD_kmer_0001424_1_TGCAGGAAATGCGAAATTTGTTTTT | 350 | TCTTTCCCTTATTTTTCCTATATTCATTCTGCTCCATTGGCGCT | T | C | Y | C | GYSNP0422 |
| CTTAATCAACTTTAGGTTCCTTTGAATTTTAGATTAGTTCT | | TATTTAACACTAACCACGTCATTGGCATGTTTTTTC[C/T]ATAA | | | | | |
| TGTTTTAATCAACTTCTGAGTTTGAT_NODE_1_length_471_cov_ | | CATTATTTTTTTTTCCTTCCTCACACATTCTTCTTATGACCC | | | | | |
| 102.513802 | | AAGTTAATATCAAACTCAGAAGTTGATTAAAC | | | | | |
| RAD_kmer_0001425_1_TGCAGGATACAAGCAAAGTGGTTA | 147 | ATACACACCATGTTTCAAGTTATAACCCTCCAAAAACCAAAA | C | C | Y | — | GYSNP0423 |
| AAATGGGAAAAAGCTTCGGAAAAAGCTTCTATATCCTTGT | | CTAGGTACAACAATACTAGTCTAGTAGTCTTGTCTATC[C/T]G | | | | | |
| ACGGATCAAATATTCGGTCGAATTAAA_NODE_1_length_661_ | | ACGAACGCACCACTGCTCGATAGTTATTGCCGCTCTCCTGGT | | | | | |
| cov_91.541603 | | GAATCCAAGAAAGGACAACCAAGCAAGATGACCTTGT | | | | | |
| RAD_kmer_0001425_1_TGCAGGATACAAGCAAAGTGGTTA | 294 | CAAGATGACCTTGTGACAACTAGCTCTCTTAATTCTAAGTTAA | G | A | G | — | GYSNP0424 |
| AAATGGGAAAAAGCTTCGGAAAAAGCTTCTATATCCTTGT | | TTTGAGGTTTTACTTGCGTACAATAAAATAAAGTCGA[G/A]CT | | | | | |
| ACGGATCAAATATTCGGTCGAATTAAA_NODE_1_length_661_ | | CATTCCTAATTTTGAATGTCCTGCTTTTGTATCGCGTATAATA | | | | | |
| cov_91.541603 | | CTACGTATCAGGTGGGACAAATGTGAGCCATACAA | | | | | |
| RAD_kmer_0001425_1_TGCAGGATACAAGCAAAGTGGTTA | 426 | ATCAGGTGGGACAAATGTGAGCCATACAAAGTAGAACACCA | T | C | T | T | GYSNP0425 |
| AAATGGGAAAAAGCTTCGGAAAAAGCTTCTATATCCTTGT | | TGGCCTAGCTTTATAAAGTAACATATATCACAATATAAT[T/C] | | | | | |
| ACGGATCAAATATTCGGTCGAATTAAA_NODE_1_length_661_ | | TTGATCTACAACATTAATCAAGTACCAAGAGGGAGTAGATCT | | | | | |
| cov_91.541603 | | AGTTTGGGTCGCAAAGAACATAATTTTCTAGTACAGGG | | | | | |
| RAD_kmer_0001429_1_TGCAGGATCACCCATTGAAGAAGTG | 286 | ACGACAATTTTGACCCGTTAACTTATGACATACGCGATATCG | G | — | R | R | GYSNP0426 |
| AAAAGAGAGTATGTTAGGAAGGTATGTGGTCAAATTTTCA | | GCCAAGTTTTCTGATTTGGTCACAGCCTTTCCTTCAGG[A/G]A | | | | | |
| CATGTATAAATGCTGTTAGAATTTAA_NODE_7_length_343_ | | GCTCCAAAGAAGTATAATGCATTATCATCTCTTTCAATGTGTA | | | | | |
| cov_56.151604 | | CTTAGTGTTTTTAATATCATGATCCCATAAAATGCT | | | | | |
| RAD_kmer_0001435_1_TGCAGGAAATGCAAGAATTAGTAG | 204 | TCGTTACATTTGGGTCCTGCTGAGTAAAAGATAACATGAGGG | W | A | W | W | GYSNP0427 |
| CACACAAATTTACATAATCCAAACCTTTGAAGAACCTGAT | | CCACAATATAAACATTGATTAATGAAGGGAAATAATGA[T/A] | | | | | |
| TTAGGAGGTTGTATGCACAAAAATGAC_NODE_2_length_345_ | | TATTGATTACACATGGACTGCTTCACCTTTTCTTGATTACTGG | | | | | |
| cov_69.953621 | | CCATCCCCCAACTACCTTAATTCATTGGTTTTGGCTA | | | | | |
| RAD_kmer_0001437_1_TGCAGGTGATGACTTTCCCATCGAC | 581 | GGTGCCTATAACAGAAATTTGACTAAAGTCGGTCACCTATTA | K | G | K | G | GYSNP0428 |
| ATGTGGTGCTTGTGCTGTTAGGTGTGAGACTCGGATGTTT | | AGGCTATTTGCCATTAAGCCCCTACAAGACAAAGTAAA[T/G] | | | | | |
| GTAACCAGTATCCTCTCTTATTTACT_NODE_1_length_639_cov_ | | AAGAGAGGATACTGGTTACAAACATCCGAGTCTCACACCTAA | | | | | |
| 78.317680 | | CAGCACAAGCACCACATGTCGATGGGAAAGTCATCACC | | | | | |
| RAD_kmer_0001438_1_TGCAGGTTGCTGACATACTCACTTA | 450 | TTAGTAGAGCTCCTATAAGAGAATTAATTATAGTTAAGCTTA | Y | C | Y | C | GYSNP0429 |
| GTGCTTTTAAAGATCAAATTCTGTAGTATTTATTAAGAGC | | AGCCTAGACGATCTGTAGATCTTTTGGGATGTTAGTAA[C/T]T | | | | | |
| AAGCTGGGTATGTTTTGACTTACATA_NODE_1_length_591_ | | CATCACTGACCGTGAGAAACATGACGACAGCCATCCACACAA | | | | | |
| cov_104.456856 | | TAGTTGATGTATAATAGTTTTTGAACATGAGGGTATG | | | | | |
| RAD_kmer_0001451_1_TGCAGGTTTTTTTCGCCATCTTAACC | 131 | AGTGTATCTTGAAACTGAACCACCACCTCAGTCGCCAAAAAC | — | T | T | T | GYSNP0430 |
| GAACTCTTGTTTATTAGTTCTTGTTAAGATCAAATATGTTT | | AATTTCCCTGGTTTTAATTAAAGTCACAACGTGCGCAC[G/T]C | | | | | |
| GTTGAGTTCATTTTTTTTTTTAAA_NODE_1_length_626_cov_ | | ACCTTAGCTTGTTCATTCAGCATATGCATAATCTCATTATGAA | | | | | |
| 102.544731 | | TAGAAGCTGACAGCAGACATGGTTTTGCCTAGCATT | | | | | |
| RAD_kmer_0001455_1_TGCAGGTTTTCACATCGTGGATGCA | 155 | GACAATAGTTGCGGTCCTGCTGCAAAATCTTGTATTACATGG | — | G | T | K | GYSNP0431 |
| CCTTTGTGTAGTTCGTTTATTTGCCGATAGCATTTTGAGAT | | GCAACGAGTATGGATACATATCTGAATATGGGAGTTTC[T/G]T | | | | | |
| ACGGGCTACCCCCATCCTTTTTGGC_NODE_1_length_408_cov_ | | GGGTGAGTCAAGTAAACGTTAAGTTAATCTTTGTCATAAATT | | | | | |
| 156.465683 | | TTGTAATATGTCAATTTGTTTGTATTTATTTTATTTA | | | | | |
| RAD_kmer_0001455_1_TGCAGGTTTTCACATCGTGGATGCA | 306 | ATTTTATTTATACACTTATATAATGTAAATTAAATTTTGCAAC | W | T | T | — | GYSNP0432 |
| CCTTTGTGTAGTTCGTTTATTTGCCGATAGCATTTTGAGAT | | CCGTGAAGTTCACGGGTTATAACCTAGTCATATTATA[T/A]TA | | | | | |
| ACGGGCTACCCCCATCCTTTTTGGC_NODE_1_length_408_cov_ | | AAAAACAATTGTAATCTGGAAATTATTAATTTAATGCCAAAA | | | | | |
| 156.465683 | | AGGATGGGGGTAGCCCGTATCTCAAAATGCTATCGG | | | | | |
| RAD_kmer_0001462_1_TGCAGGATGATGCATATATTTATAT | 170 | AAACTAAGAGCATGTGCAACAGGAACTTCATCGTTTACCTTC | Y | T | Y | T | GYSNP0433 |
| GTGATTATATTCCTTAATTAATATATAAATGAATGTTGGA | | ATACTAGACGATCCTGCTGGCAACAGCTTTATAGAAAA[T/C]C | | | | | |
| CTTATGACATGTAGAAGATTATAGTA_NODE_1_length_612_ | | TGTAACTTTAACTCTGTTGTATTCCTTTTCCTTTTAGCAAGCTA | | | | | |
| cov_151.772873 | | TATCATTTTTTCTTTGTACCATACCAAATGCACAA | | | | | |
| RAD_kmer_0001462_1_TGCAGGATGATGCATATATTTATAT | 324 | TGCACAACACGTGTTGCATTTTGTTAAAATATATGACAACTTT | M | M | M | M | GYSNP0434 |
| GTGATTATATTCCTTAATTAATATATAAATGAATGTTGGA | | GATTTTGTAATAGGTATGTTCCATCCCCAGATCCGTC[C/A]TT | | | | | |
| CTTATGACATGTAGAAGATTATAGTA_NODE_1_length_612_ | | AGCATTGATTTCTACGAGCGAACTCCCGAGCAACAAGCTTC | | | | | |
| cov_151.772873 | | GCTTGGATATCTTGTGGACCAATCACAAACGAGAGA | | | | | |
| RAD_kmer_0001463_1_TGCAGGCAGCTAATCAGTAACAATT | 204 | TACATTCTAATTGGAAAGTCTTATGACATCATATTTACTTGGG | A | G | A | R | GYSNP0435 |
| ATGAAGATATTATAACAGTTGTTTTTTTCCTAAGATTCAA | | CACTCTTTAGGATCCTTGATACTTTTAAAATTCTAGC[A/G]AA | | | | | |
| ATGTTCTTGACATATAGAACCAACAA_NODE_4_length_369_ | | ATTAGACAAGAGTCATTATCTTTCCCTTAAGAGTTCTTTTATC | | | | | |
| cov_33.319782 | | ATCTACCAACTTGATTACATTATTAAAATCTGTTC | | | | | |
| RAD_kmer_0001464_1_TGCAGGGATAAAAATTTCCTCATAT | 189 | ATTTAATTTCAGATTTTCAGTGAACACAAATAGTAAAAGTGA | Y | Y | C | Y | GYSNP0436 |
| TAAGAACAACGTGCTAGTGTGACACATGAAGAATAAATA | | ATGGGAAACGGTGCTTTGCTCTCACATTGTTCTGAAAA[GT]A | | | | | |
| AAAGTGACATCTTCCCATGACAAGTTT_NODE_1_length_295_ | | ATTGTTACCCATAGGATGGTGATGGTGTTTATTGGATGAGTAT | | | | | |
| cov_91.247459 | | CGTTCATACTTCATAGGTTGTTAGATTATATGGCAC | | | | | |
| RAD_kmer_0001465_1_TGCAGGTGATATGTGTATGCTAAAC | 424 | TCTTGCTCATGCTTTAAGAAAATTGAAGTATGTCTACTTTTTT | C | T | Y | Y | GYSNP0437 |
| CAGCATATGTGCTTAAGCTTCTTGTTGATAGTGCAAGGGC | | TATTTCCCAAACTCGTGATTAAATGGTCCGACTGTTT[C/T]TAA | | | | | |
| CTTTTCGTAATATATGATTGCTTCAC_NODE_1_length_539_cov_ | | ACTTACTTTTGTCCAAATTTTTGTACAACTCTGCATCAGGAGG | | | | | |
| 158.651199 | | TACAGTGAAGCAATCATATATTACGAAAAGGCCC | | | | | |
| RAD_kmer_0001467_1_TGCAGGCTGCGGCTTGTTTCTGGTT | 152 | ATTCTAATAAGAAAATTTACTATCTAGAAATATCAGATTACA | W | W | A | — | GYSNP0438 |
| GTTGATGAAGGGGCCTTATTCAAAAAGGCGTACAACGCA | | AAGCAATGATGCAAAAGGAAAGGGACGCTGTCTGAAGG[A/T] | | | | | |
| ATGATCATCATCTGGTGCTCCGAATCG_NODE_1_length_487_ | | GACAGCTAAAAAACAACTTCTTTTTTTTATTTATTTTTTATTTT | | | | | |
| cov_131.576996 | | TTTATAAATCTAAAGTTTTTATTCATATTCTTCACA | | | | | |
| RAD_kmer_0001473_1_TGCAGGCAACAATATTAATCAACAC | 347 | TGTAACATGTAACATCCATTTAATTTTATAGGGGTTGGTTGTT | W | T | W | A | GYSNP0439 |
| TGCTAAACAACAGGACCAACAAATTCTATAACATAAAAC | | GATTCTGCTTTACAAATGGATCAGTCTTCGGGTTCGG[A/T]TTT | | | | | |
| TAATAAATGCAATTAAAGTAATCAAAT_NODE_1_length_532_ | | TGATTACTTATTCAAGCTGTTGATGATTGGGGATTCAGGGGTT | | | | | |
| cov_109.501877 | | GGTAAAAGCTCTCTGTTATTGAGTTTTACTTCTG | | | | | |
| RAD_kmer_0001477_1_TGCAGGCCTAAATTGGTTAACTCTA | 109 | AATCGCTTACAGTTTTAGGTTTGTTTAGTTAGTGTATGTGATG | G | R | A | A | GYSNP0440 |
| GGATACTATGTTTATAGATATTATATCATTGTAATTTATTG | | AGATTTTTTGCCAAGGGAAATGGCACGACCACATGTA[A/G]A | | | | | |
| AACGTGTCTATGGATTCAAAATAAA_NODE_1_length_510_cov_ | | TCGTTGGCAATTGGCATCTTGCCTAGCTTGCATGAGAACACA | | | | | |
| 91.364708 | | TATATAAAAGCGATATACATTTGTTGAAATAAAAGGT | | | | | |
| RAD_kmer_0001479_1_TGCAGGCCTTATTGAATTCTTGGCTT | 290 | ATATATAGATAGATACCAACATATAATAATACTACCATGCGC | K | T | K | G | GYSNP0441 |
| TGACTTTTCAGATTTCTATCTTTAGTTACATTACTCTCAAC | | CACACAGTCTATGGCAACTATTCAACAAATTGTACCGG[G/T]T | | | | | |
| TTTCATGTTGCGGTTTATTAATTT_NODE_length_559_cov_ | | AACTATCCAAGATCTTGTTGAGGCACGTCATTATGAAATTCT | | | | | |
| 122.500893 | | AGTAAATAACAACCCAAGGTAAACCCACCATAATACG | | | | | |
| RAD_kmer_0001491_1_TGCAGGATTGAACAAATATAGCGA | 84 | ACAGTGTTGTCATTACCACCAGAAGCCAGTTTCTGAACCGGT | — | T | T | Y | GYSNP0442 |
| ACAGACGTCAATAGCAAATCATGCCAGGGCAGAAGATTG | | TCAAACTCGCCGGACCCGATTAACGAACCAGGCTCCAT[T/C]G | | | | | |
| AAACAGGTCACTCAGACATCATCCATGA_NODE_1_length_ | | AAGGAGCCCAGGTGACGGAGGTTACCCCAACCGGATGAGCT | | | | | |
| 572_cov_97.919579 | | TGGTCTATCTTTGTGGTGTCCCACCCACCATCTGACCT | | | | | |
| RAD_kmer_0001494_1_TGCAGGCGTTGCATGTGCTTTAACT | 94 | CACTTCATATGGGTCAAACAGGTTGAAAGTCGCTTAATGTGT | R | A | G | A | GYSNP0443 |
| TGGCCCGAATTATATTGCAATGTTGCCTCTAACGTAGATT | | TATTTTAATGTATATAACCTCCTAAATAACAATATTCA[G/A]G | | | | | |
| CTGCCATTACAGCCCTCTTCTCCATT_NODE_1_length_632_cov_ | | AAATTATATTATTAAGGAGATATTATGATTCCTACAATCATAT | | | | | |
| 89.726265 | | TTGATGCGGTMGTATTTTTAGAAAAAATTGAACCC | | | | | |
| RAD_kmer_0001494_1_TGCAGGCGTTGCATGTGCTTTAACT | 166 | AATATTCAGGAAATTATATTATTAAGGAGATATTATGATTCCT | T | T | A | T | GYSNP0444 |
| TGGCCCGAATTATATTGCAATGTTGCCTCTAACGTAGATT | | ACAATCATATTTGATGCGGTAAGTATTTTTAGAAAAA[A/T]TT | | | | | |
| CTGCCATTACAGCCCTCTTCTCCATT_NODE_1_length_632_cov_ | | GAACCCATAACAGAAAATTACAGCATAATTTTATCATATTTG | | | | | |
| 89.726265 | | ACACACGAATTTCAAACTTATTTTCAGTGATATTGC | | | | | |
| RAD_kmer_0001494_1_TGCAGGCGTTGCATGTGCTTTAACT | 294 | CACGAATTTCMACTTATTTTCAGTGATATTGCAATATCATGC | S | G | C | G | GYSNP0445 |
| TGGCCCGAATTATATTGCAATGTTGCCTCTAACGTAGATT | | ACACATTTATTTGAGTTATGTTTATACATGACCATAA[C/G]TA | | | | | |
| CTGCCATTACAGCCCTCTTCTCCATT_NODE_1_length_632_cov_ | | CCATTATTAAAAAGAAGGGTTCTTGATTATAAGTTTTGGTATT | | | | | |
| 89.726265 | | CTACAACTTTGCCCTCATTAACGAAGGTGATAATC | | | | | |
| RAD_kmer_0001515_1_TGCAGGGTAATACTAGGTGTTGTAA | 342 | CTGCAAGTAGAGCTGACATGGCCTTAACTGATGGTTTCTCTCC | — | G | G | K | GYSNP0446 |
| AGAGGGGATTCCTTTGTATTGGATTGCCCCAAGGTGTTAG | | TCCACGTCAGCCACCTCTCCTCCAATTGTCTTCCACA[G/T]TTG | | | | | |
| TTGTTGTGTAGATATTGTTATCTTTC_NODE_1_length_640_cov_ | | CATCCGCGTGGGTCGATAGAACCCCTTGCAGATGCTCTAATC | | | | | |
| 113.334373 | | TTTGTTTCCATCATGTTTATATCATGGGTTCATGT | | | | | |
| RAD_kmer_0001517_1_TGCAGGAGGAGCGGCTGGTCAAAG | 219 | CAAAAAAAAAAGGTCAACTGTGAACAAGAACTAGTGAAAAC | A | — | T | A | GYSNP0447 |
| TACCAGAAGAAGAGGTACAAACATTTTCTATTTAGTTTTT | | CAGCTTCTTAAGGTTGAACCTTTTGCAATTACGAACTCG[T/A] | | | | | |
| CTGACATTTTCTAATGTTTTATGCCAA_NODE_1_length_585_ | | GTTAAGTAAGATTAACACATCAAAATTTTACGATCTTGAAAT | | | | | |
| cov_69.543587 | | CTTGCATGGACCCTTTAAAGTGAAAATAAAAAATCAAA | | | | | |
| RAD_kmer_0001517_1_TGCAGGAGGAGCGGCTGGTCAAAG | 321 | AAAATTTTACGATCTTGAAATCTTGCATGGACCCTTTAAAGTG | G | — | G | A | GYSNP0448 |
| TACCAGAAGAAGAGGTACAAACATTTTCTATTTAGTTTTT | | AAAATAAAAAATCAAACAGCGATAAACAAGAAGAAGT[G/A] | | | | | |
| CTGACATTTTCTAATGTTTTATGCCAA_NODE_1_length_585_ | | ACAATCTATAAAACAACATAAATCAGAATTACCTATACGTTC | | | | | |
| cov_69.543587 | | AATGACACTCTTAGTAATGTAGTTTGATAGCATTTTCC | | | | | |
| RAD_kmer_0001522_1_TGCAGGCACAACCTCACCACCATTT | 525 | GCAAAACAAGTAGAACAAACCAAAAAAAAGAGTTTATTGTT | T | T | Y | C | GYSNP0449 |
| CATTTTGACAACTTTTAACTTTTTTATTTGTCACACTTTGTT | | AAGTGGAAATTGATTCATACTGATTGCTAAAATAATGTT[C/T] | | | | | |
| TTATCGTTACGTCTTAAAACAAAT_NODE_1_length_618_cov_ | | CTACAGTAGAATGTTGGATGATCCATTTGTTTTAAGAC | | | | | |
| 100.579285 | | GTAACGATAAAACAAAGTGTGACAAATAAAAAAGTTAA | | | | | |
| RAD_kmer_0001532_1_TGCAGGAATATGCTGAAGAACAGA | 367 | TAAAATTAAACCGACTGAGCAAAGCAGGTAGACGTGTAAGA | G | C | G | S | GYSNP0450 |
| ACAATGGAACATCCTTTAATGATATGAAATTTATTTCAAA | | GAAAACAGTGAGAGGTGTTCCATTATACAGTAGAAAATA[G/C] | | | | | |
| AAATGGTCACTCTAAAGAACJATTTCT_NODE_1_length_624_ | | CATACAACCAAGTAAGATTATTTTAACTGCACAACCAATTA | | | | | |
| cov_89.689102 | | TGGATACCGTAAAACACATGATAGTCAATATATGATAG | | | | | |
| RAD_kmer_0001535_1_TGCAGGCATAGAGTAGAAACTAAC | 445 | TCGAAGTAAACGTTTGTTTGGTGTTTGTAGAGACTAAGACAC | R | G | R | G | GYSNP0451 |
| ACATACGCACCATGTTCAACTTGGAAGGTGTAACCGTTCC | | ATATTGGACATGGGTCGGAGCATTTCTTCTCCTGATGG[G/A]C | | | | | |
| AGCCACGGCCATTAATGAGGAGCCCAT_NODE_1_length_505_ | | TCCTCATTAATGGCCGTGGCTGGAACGGTTACACCTTCCAAG | | | | | |
| cov_94.184158 | | TTGAACATGGTGCGTATGTGTTAGTTTCTACTCTATG | | | | | |
| RAD_kmer_0001542_1_TGCAGGCCCACCAGCTGCAGCCCAA | 537 | AATACAACAGTCAACAAAGAATACTTCACCCGAAGCGAGAT | T | C | T | C | GYSNP0452 |
| CCACCAAAACTATTGAAGTTTGTGGACACAGTGGAAATG | | CTTCCCTTTATCGATAACAAGTCTAGCACCCGAAACCAC[T/C] | | | | | |
| ACAAGGGGCCCGCAAGAATCACCGGGT_NODE_1_length_607_ | | CAATAACCCGGTGATTCnGCGGGCCCCTOTCATTTCCACTG | | | | | |
| cov_1_17.542007 | | TGTCCACAAACTTCAATAGTTTTGGTGGTTGGGCTGC | | | | | |
| RAD_kmer_0001545_1_TGCAGGTTTTCCGTGGTTACCAAAA | 92 | TATTGTTTTCATGTTCTTGCCTATGCAGAGTGGCTTGTTAATA | G | — | R | A | GYSNP0453 |
| AAAAAGATTAAAAAATAGCAAAATGAAGTTATGACTTTC | | TGTTTACTTCAAAAATTATGACCATTTACGAGCGTAT[G/A]GA | | | | | |
| ATGAAAATCTGTTCAGTTATGAATTAA_NODE_1_length_588_ | | AGTATAGTTAAGGACCTGAAGAGTCCAAACTCCTTTTGGGCC | | | | | |
| cov_76.767006 | | TCGAAAATCAGGTATTCCACAAGAAGTTGATATTCC | | | | | |
| RAD_kmer_0001546_1_TGCAGGATCTGTATTGTCGATGAAC | 392 | ACTGAATTTTTATTTGGCAAAGCGCGTTAGTTGTGGCTAAAC | — | A | C | A | GYSNP0454 |
| CTTATCTGGTAATCAATGAACATAAAAAATTTGTAATTTC | | AAACATGATGTGATAACTTAAAAAAAATATATGTTAGC[C/A]T | | | | | |
| AACAAGAATGGATAGAAGAGAAACAA_NODE_length_491_ | | GCTTACTTCAAAGTGCTCTTCTGTGTTCATCTTTTGTTTCTCTT | | | | | |
| cov_S9.769S59 | | CTATCCATTCTTGTTGAAATTACAAATTTTTTATG | | | | | |
| RAD_kmer_0001555_1_TGCAGGCGCAGTTAACATTAGCACG | 260 | GGGTTTGAATGCCATCCCTATGTAAGAGTTACTAAGAAAGTT | A | T | T | T | GYSNP0455 |
| TTATGCCTCAACACCACTGCTCCTCTTATATATGACAAGT | | TGATTTGATCCATTTACAATATATAAATGATAAGTCTA[T/A]T | | | | | |
| GTTGGTCAGACATAAGATTTATGAAA_NODE_1_length_587_ | | AGTTATCAAGGTTTTACAATTCTAAAATAATTCATAAGGGAT | | | | | |
| cov_90.066437 | | TGCTTAAAGGTTATTCATCTAGCATTTTTTTTATATT | | | | | |
| RAD_kmer_0001565_1_TGCAGGAATATATCACCGATTCACC | 264 | CAAAAATCACCAAAGAGTTATTTTAATGCATACATGTATGCA | T | G | G | G | GYSNP0456 |
| CTCGACACCTCCAACTATATTAAGGTAAAGAGTGCTAGAT | | ACCTCCTAAGTCATTTTATTTAAGAAATTAGATTAGTA[G/T]T | | | | | |
| TGTGAAAAAGGGGTGGGTCAAGCTGG_NODE_1_length_482_ | | GTAATAATTTAGTTTTATAATCATACTTTAACACGCTAATTAT | | | | | |
| cov_96.647301 | | TTATATATATTCACAAGATTTGGACAAAAGGTGTTC | | | | | |
| RAD_kmer_0001565_1_TGCAGGAATATATCACCGATTCACC | 347 | TAATAATTTAGTTTTATAATCATACTTTAACACGCTAATTATT | G | C | C | C | GYSNP0457 |
| CTCGACACCTCCAACTATATTAAGGTAAAGAGTGCTAGAT | | TATATATATTCACAAGATTTGGACAAAAGGTGTTCCC[C/G]GT | | | | | |
| TGTGAAAAAGGGGTGGGTCAAGCTGG_NODE_1_length_482_ | | CAATTCAACCTGACACATACCAATATTACCCATTTTGAGTGA | | | | | |
| cov_96.647301 | | CCGAAACCAATTTGCCCGTTGCCCAACCAGCTTGAC | | | | | |
| RAD_kmer_0001569_1_TGCAGGCCCATTTACTGTTCTTCATA | 307 | TACTTCATGGAGCCTACGTGTAGAAGAAGTGTATTTTAGGGT | — | T | C | C | GYSNP0458 |
| CGACTCAAATTTCTGGCT1TATAACTAAGGGCCTGTTTGTT | | TTTGCATGATGTATAGAAGGTTAGGGGCTGTTTGGCAA[C/T]T | | | | | |
| TCAGCCTCTTAATGGTTCAGATGr_NODE_1_length_457_cov_ | | TCTGAATGGGTAAGTGCTGAACCAGTAAGAGGTCTGAACCAT | | | | | |
| 39.199123 | | TAAGTGCTGAACCAGTAAGAGGTCTGAACCATTAAGA | | | | | |
| RAD_kmer_0001575_1_TGCAGGTCGACTAAAGCTGCTCAAG | 449 | AGTCAAAATTTTGTTTACTGTAAAAATTACACCAGCTTAATTT | Y | C | Y | — | GYSNP0459 |
| AGCCCAATCACAGAGAACCATTAAATACTATAAGGTATTC | | TGTTTAATCATATATTTTGTTTTCTAATTTAATTATC[T/C]ATTT | | | | | |
| CATCTCTCAGCTAACTTTTAGTTGCC_NODE_1_length_570_cov_ | | TCAATAAAGTTACCCATAATCATATCAGTTTCATATATCAAAC | | | | | |
| 1O3.263161 | | ACGAAGTAAGGCAACTAAAAGTTAGCTGAGAGA | | | | | |
| RAD_kmer_0001581_1_TGCAGGGTCTACCCATGGTGGTCTC | 338 | ATCTAATATATTTATAAGAACATCTTTCACTGAATACAAGAG | T | K | K | T | GYSNP0460 |
| ACTCATGTGAGTTTTATTCATATACCTTCATCCACAATTCA | | AACATTATGTGCTAACATGATTCACGATAATGATGTTT[T/G]T | | | | | |
| AATGAAATAACATAGCAACTTAATT_NODE_1_length_569_cov_ | | GTATTCATTTATTCAGTGAAACATCTTGTTATATGTAACTGGT | | | | | |
| 136.351501 | | TTTATAGTATTTATATACTATGAATATATGAAACGA | | | | | |
| RAD_kmer_0001584_l TGCAGGCCCACAGTTGATTCGCTCC | 242 | TGGAGGCATTAAAACGAGGAGAAAAGCCACCGGTAAGTTAA | T | C | C | Y | GYSNP0461 |
| GGGTTTTGGGTCCCACGCTATCAAAGGTCAACATAAACAA | | AAAATGGGTGGGTCAGGTAACGGGTCAAAACGGGTTGGT[C/T] | | | | | |
| GTTAATTAGTGTCTCGGTGAACAATT_NODE_1_length_528_ | | GGTATGGGTTGAGCCACTTACACATGTATGCATTAAAAATAG | | | | | |
| cov_83.071968 | | ACTTTTAATAAATTCCTAATTTTTTTTTTCGAACAACA | | | | | |
| RAD_kmer_0001586_1_TGCAGGTGTAGGCATGGGTATTTCC | 107 | CCGAGTGATGATCAAAACTTTCATAATCGACTCTTTACAACTT | G | G | T | G | GYSNP0462 |
| ACGTTGTAAATAACGATTACACGCACTGGGAAATGTATGC | | ACAACATTAACCTTGAGCAAATAGTAATTACATAGCA[T/G]CA | | | | | |
| TATTGGTGGCAGTGCGGATCCGACTA_NODE_length_629_ | | GGAGAACACTCAAACTTCCCTTTCCCGGTGACTTGATCGTGTT | | | | | |
| cov_75.453102 | | CAAGGCAAGCATATGAGCGAACGAAACAGAGAAAA | | | | | |
| RAD_kmer_0001592_1_TGCAGGATACTAAGAGTACAAAAC | 382 | AAAGCATAGAGACGGAATCAGTTTATTCTCTAAAATGCCACA | G | G | C | G | GYSNP0463 |
| CAAGATGCGTCTAAGAAATCATTCCCTCCTCCCTCTTTCCT | | TATCACAGGAGGTGAATCATTTGCATGAGGGCTCTGAAA[C/G] | | | | | |
| TTGTTTTTTATTGTTAAATAAGTCAA_NODE_3_length_439_cov_ | | ATGTAAGTTTTATTGTAGAAGTGATCACGCTTGCAAATAATCT | | | | | |
| 45.264236 | | TTTCCTCCATCTCTTTTTTTTGTCTCAAACAGATATG | | | | | |
| RAD_kmer_0001595_1_TGCAGGCCAAAAATGATCCAATTGC | 294 | ATGGGTAATTAAATCATATAAAGTACAAATAAATGCCAATGA | A | A | G | G | GYSNP0464 |
| TCCTGATAGAGCAATACCACGTGAAGATATTAAGGTATGT | | ATCTGATCTATGCATTTACTGATTGCAGCAAGTTGCTA[G/A]T | | | | | |
| TCAATGTAGCACAATCATGTGACAAG_NODE_1_length_567_ | | ATCTTGTTGTGTGCCCGATTTTTCATTTTCAAGATGTTGAAGG | | | | | |
| cov_20l.938278 | | AAATCCATGACCATCGGATCGGTCCATGGACATCCA | | | | | |
| RAD_kmer_0001612_1_TGCAGGTTTTGATACCACTAGTTTG | 144 | TTTTAAATTAGGTTAAAAGCTCGTTTACTACACGAGTTGAA | R | G | R | K | GYSNP0465 |
| ATAGAATAATAAGCAGGTTCTCCAATGTTTCACTAAAACA | | TAAAAAAAATATTAAATACTTAGTAATLGAAGATTTTT[G/A, T] | | | | | |
| TTCATTATTACTCATTTCTTATTTTT_NODE_1_length_629_cov_ | | AATTTTAAAAGATATTTATTGATAAGACATTATTTAAATTACG | | | | | |
| 70.653419 | | TATAAGTAAGTAATTTTAATAATAAAATGACTATTGA | | | | | |
| RAD_kmer_0001618_1_TGCAGGAGATTTACTATTTTCTGCA | 313 | GGGAGTCGATGGCCCTGCTACTTGTCGTCGTACAGACAGCGG | A | G | G | G | GYSNP0466 |
| GCTGATGCCTTTTGAAAAAAAAAATATATATATTATTTGC | | GCACACCTCGTCGCTTCTAGTCCGGATTCTGACTTAGAG[G/A]C | | | | | |
| CATGCTGATATACAAGGAGCCCATGT_NODE_1_length_607_ | | GTCCAAAAGTCACCTAGAAGAACGATCTGGATCACTTTTACA | | | | | |
| cov_79.510712 | | CTTAATGGGTTGACTGGTCATCTAGTGGAACCTTCAT | | | | | |
| RAD_kmer_0001619_1_TGCAGGTAGTCGAGATTCTCAAACA | 152 | GTAGATGCAAGCTCACCATTGCTTTTAAGCTCCAAAATGATA | T | K | T | K | GYSNP0467 |
| AGAAAAGGTGGATTTCAAGACTTTTGATATACTCTCGGAT | | TCACATCCTCCAATCAACTCACCTTTATAATAAAGTTG[T/G]G | | | | | |
| GATGAAGTAAGGCAAGGCCTAAAAGT_NODE_length_553_ | | GGAAAGTAGGCCAATTCGAGAACGTTTTTAAACCTTGTCGAA | | | | | |
| cov_113.670883 | | CTTCCTCATCGCTCAAAATGTCAAAAGATCCAAACTT | | | | | |
| RAD_kmer_0001621_1_TGCAGGTTGCTATTAATTCCCTCATT | 325 | GAAAATTGAACTTGTGTTCATATGAAGCTATGTTGCCTTTGGC | R | A | R | — | GYSNP0468 |
| CGGATACTTAGGGACCCATCGCTTTCTAGTTTATCATCAAA | | TCATAGATAGGACAATTAAAGATTAGAACAATGTTTC[A/G]G | | | | | |
| AGGTTGTTGGGTCACTCATGTTTAT_NODE_1_length_558_cov_ | | AATCCCATGCTAAATGAACTAACCTTTGGCAAGTATGGCACG | | | | | |
| 161.360214 | | CAGCCTAGCCCCATTGACTGATCTTGCACAGATAAGT | | | | | |
| RAD_kmer_0001629_1_TGCAGGTGCTATGCCAGAAAAATGA | 383 | TGATCTCAATCGATGTTATGAAAGCCTGGGAGAAGCAAAGTA | C | C | A | C | GYSNP0469 |
| GCATGCTCCTTAGTTAGTTTATCTTCAGTCTTCTGAATAGC | | ATGACACCAAAAACAATCAATTATGTCTCCTTTACGAT[A/C]C | | | | | |
| CACATATTCGAATATATATATATAT_NODE_2_length_479_cov_ | | TCCACGTTCAGCATCAGTTGCTGTTTCCATTTTCCTGGATTGT | | | | | |
| 57.511482 | | CATTTTCACCTCGTGCTTGCCCATCTAATAATAGAT | | | | | |
| RAD_kmer_0001635_1_TGCAGGTATTGAACTGAAGAGAAG | 126 | GTGAAAGCTCATTAAGAAAAGGGGCATGGCTAAATGTTTCAC | G | G | R | — | GYSNP0470 |
| CAAACTGCATATTGCAACAGTTATTGGGTACCTTATCA | | CAATGGGTATTTAACGATGAATCTCAGTTTTTTTCTTA[A/G]TT | | | | | |
| CCCGTCAAGATGAATGGATGTCAGGGT_NODE_length_584_ | | GTGATGGAGATTCTGCCACAAATTTGACGCTAAAACTCATGC | | | | | |
| cov_122.330482 | | TGAATTTCAATATTTATTTTTTTAGTTTTACTAGTT | | | | | |
| RAD_kmer_0001635_1_TGCAGGTATTGAACTGAAGAGAAG | 178 | TTAACGATGAATCTCAGTTTTTTTCTTAATTGTGATGGAGATT | T | T | Y | — | GYSNP0471 |
| CAAACTGCATATTGGAACAGTTATTGGGTAGCTTATCAAA | | CTGCCACAAATTTGACGCTAAAACTCATGCTGAATTT[C/T]AA | | | | | |
| CCCGTCAAGATCAATGGATGTCAGGGT_NODE_1_length_584_ | | TATTTTTTTAGTTTTACTAGTTTTATTCTATTTATAAATA | | | | | |
| cov_122.330482 | | TTTCTGAACTTACACACACACACACACGGAGATA | | | | | |
| RAD_kma_00l642_1_TGCAGGTCAAACCACCGTTTAAAAG | 113 | ATTCGAGTTGCTTGCGAACTCACGAGCCGAGCCATACCTAGC | G | — | S | G | GYSNP0472 |
| ATATTCTCCACGTACCTTATAACTCCATGACAACACCGAG | | TCGAGCTTGGGTCGCTTACAACCAAGCTTTTTTCAAAC[C/G]A | | | | | |
| GGATTGTAAGCTTTGGTGACCGTCAA_NODE_1_length_385_ | | GTCACGAGTTGCGAGCTTCTTTGCAGATATCATGATAAAATA | | | | | |
| cov_120.171425 | | GTTGAGGTGCTATAGGTGTATAACTATGAAATAGAGA | | | | | |
| RAD_kmer_0001643_1_TGCAGGAGGCAGAGGAACTCCGGT | 566 | ATGTAATGGTTCTTAAGAGTTATTTTTGTTTTGTTTTGTTTTGT | G | T | G | G | GYSNP0473 |
| AGCTGCAAAACCAAATAAAGAATAAAAAACTGAGAACTT | | TTTGTTATTTTATAAAGTTCAGTTTTTTTATTTTTT[G/T]TTTTG | | | | | |
| TATAAAATTACAAAACAAAACAAAAAAT_NODE_1_length_ | | TTTTGTAATTTTATAAAGTTCTCAGTTTTTTATTCTTTATTTGG | | | | | |
| 623_cov_74.792938 | | TTTTGCAGCTACCGGAGTTCCTCTGCCTCCT | | | | | |
| RAD_kmer_0001644_1_TGCAGGAGCTCAAGTACCGTGTCAT | 85 | ATACTTGGCTGCAGTAAACCTTCAGGGTGTAAATTTTTTAAA | A | R | R | G | GYSNP0474 |
| AATAAAGTGGCAACCTCGCCTGCGATACAGTGACATGTAC | | ATAAGGTCCACJATCATTATGGAGTCAGATTAATAGCC[A/G]A | | | | | |
| TACTTTGATATTGACAAACATGTAAG_NODE_1_length_434_ | | TTAAGTGGGGCATGTATTTCCGACTCATACGTACACCAATGT | | | | | |
| cov_46.502304 | | TGTGTTGTCTACCTTCATCTAATAAGTTTAAAATTA | | | | | |
| RAD_kmer_0001644_1_TGCAGGAGCTCAAGTACCGTGTCAT | 261 | TGAATACTAATTCAACAATTCGTTTTAGTTTTACCTTGGTATT | G | A | R | A | GYSNP0475 |
| AATAAAGTGGCAACCTCGCCTGCGATACAGTGACATGTAC | | GTGTTACCCTTGATGTAAGAACCATGATTTTTCAAA[G/A]GC | | | | | |
| TACTTTGATAHGACAAACATGTAAG_NODE_1_length_434_ | | ATCATCTTTGTCATACGTGAAAACCATTTATTTCCATGATCAT | | | | | |
| cov_46.502304 | | ATTTCAACAAATGATTATTTTACCAAATTATAGCA | | | | | |
| RAD_kmer_0001644_1_TGCAGGTAAGTATCTTTATAAGTAA | 270 | GTTTGCXGTGAGTGTGCCTGTATATGTTGCAATGCTGACTTGC | S | C | S | S | GYSNP0476 |
| TAGCTAAGTAGTTGACCGAAACACTTTTGATATATTTCTTA | | ATTAGTGAGTTTGTTTGGTGAAGCTAAGTCACTATTA[G/C]TTT | | | | | |
| CTATCCAAAGAAAAAATATTGATTA_NODE_1_length_589_cov_ | | TCTTTTAGCATTATTGGAACATAAAAAGTCACATATTACACA | | | | | |
| 133.755524 | | AGTTACTGTATTGTGTAAAATTGAGTTATGATATT | | | | | |
| RAD_kmer_0001647_1_TGCAGGTAAGTATCTTTATAAGTAA | 323 | TTGTTTGGTGAAGCTAACTCACTATTAGTTTTCTTTTAGGATT | R | R | R | G | GYSNP0477 |
| TAGCTAAGTAGTTGACCGAAACACTTTTCATATATTTCTTA | | ATTGGAACATAAAAAGTCACATATTACACAAGTTACT[G/A]TA | | | | | |
| CTATCCAAAGAAAAAATATTGATTA_NODE_1_length_589_cov_ | | TTGTGTAAAATTGAGTTATGATATTTTCTTAACTTTGTAGATA | | | | | |
| 133.755524 | | ATAAACTATGGTTTCAGCCTGAATGCACCGGTAGT | | | | | |
| RAD_kmer_0001647_1_TGCAGGTAAGTATCTTTATAAGTAA | 496 | CAAACGGCGCCCAGTCCTCGAGCATTTCATATTGCCATTGCA | C | Y | C | Y | GYSNP0478 |
| TAGCTAAGTAGTTGACCGAAACACTTTTCATATATTTCTTA | | ATTGACTGCCATATGTTTGTTTTTGGAGGGAGACTTGG[C/T]T | | | | | |
| CTATCCAAAGAAAAAATATTGATTA_NODE_1_length_589_cov_ | | CCAAAAGGTTGTTCAACTATCAACCTTTAATCAATATTTTTTC | | | | | |
| 133.755524 | | TTTGGATAGTAAGAAAEATATGAAAAGTGTTTGGGT | | | | | |
| RAD_kmer_0001649_1_TGCAGGTTTTTAAGTTCTTATGGTA | 302 | TTTGGTAGTTGAACAATAAGCAGTTAGAGTAAGAAACAGTAA | T | C | Y | Y | GYSNP0479 |
| AGATGACATTGGTCCCCTCCTCTCTTTATGTAGTCATCCCA | | GTTTATAAGATGTCCCTCTTCCTACAAAAAAACTACAT[C/T]T | | | | | |
| ATATGCATCATTCTTCTTCACTACC_NODE_1_length_543_cov_ | | TCAAAAAACTAAACTTTATCACTACATTAATGATATATTTACA | | | | | |
| 206.773483 | | TATTTAAATTACAAAAAAATAATCAACTACATATAT | | | | | |
| RAD_kmer_0001649_1_TGCAGGTTTTTAAGTTCTTATGGTA | 414 | TGATATATTTACATATTTAAATTACAAAAAAATAATCAACTA | G | C | S | G | GYSNP0480 |
| AGATGACATTGGTCCCCTCCTCTCTTTATGTAGTCATCCCA | | CATATATTAAAACTATAGGTCATATGTGTATATTCAGT[G/C]A | | | | | |
| ATATGCATCATTCTTCTCACTACC_NODE_1_length_543_cov_ | | ATGAATGTATAGCTAGTAAAAATGAATCTAATAGGATAAGGG | | | | | |
| 200.773483 | | GTGTTGAGATGTGTTAGATATGGTAGTGAAGAAGAAT | | | | | |
| RAD_kmer_0001651_1_TGCAGGTAATTGGATCAAATATCGA | 157 | AGATTTGTTAATTCAATTAAGCAACATACGAATTGTGTATAC | T | Y | T | Y | GYSNP0481 |
| CAGGGATAGAATTCCTGGCATTGATATCAGCTTAAATGAT | | ACACATGGATCACCATTCAATAATCAACAGCACAGTGA[T/C]T | | | | | |
| GGTGACACATGGATGTTTGCTGGGCA_NODE_length_456_ | | TACAATGAAACTCAAATAACTTGTAACAATATTGTGTGTAAC | | | | | |
| cov_49.353069 | | AATGCCTCGAGAATCCACATAACTTTATAGTAGTCCA | | | | | |
| RAD_kmer_0001651_1_TGCAGGTAATTGGATCAAATATCGA | 229 | CACAGTGATTTACAATGAAACTCAAATAACTTGTAACAATAT | A | R | R | A | GYSNP0482 |
| CAGGGATAGAATTCCTGGCATTCATATCAGCTTAAATGAT | | TGTGTGTAACAATGCCTCGAGAATCCACATAACTTTAT[A/G]G | | | | | |
| GGTGACACATGGATGTTTGCTGGGCA_NODE_1_length_456_ | | TAGTCCAGATTGGTAGTTTTGTTGATAGAAAATATTAGAAAA | | | | | |
| cov_49.353069 | | TGAAGTTGCTACTTAACAAGTTCTGTATTTGGGACAT | | | | | |
| RAD_kmer_0001655_1_TGCAGGATGTTAATGAAGAGGTGA | 83 | TCTCCAATGCTTCCAACTTCCTTAAAAATCTTTACTCATCCAG | T | T | T | C | GYSNP0483 |
| ACCAACTTATGATAAAAAATTATGATTTTCATAAGTTTGA | | TCTCGTGAAGTAGAGATGTTGTAATAATATATAAGAT[T/C]CC | | | | | |
| TCTCTATCATGAGGACAACTTTTTTTG_NODE_2_length_503_ | | TATAAGAACAATAAATCAAACAATTAGAACAAACATATATA | | | | | |
| cov_59.838966 | | AGGTTATGTGTACATATAAAATGATTTTTCTCTATTA | | | | | |
| RAD_kmer_0001657_1_TGCAGGATGAGCAAAAGATGCATC | 362 | CCTAAAAGGTATTAAACCTGCATTTCTTGATCATACCTGTATT | T | C | T | T | GYSNP0484 |
| CAGTTAGGCGCGTATAATATATAAGGAAGCAAATAGTAA | | TTCTTTCGTTCAAATGTTTAATTCATTGATGTCATTG[T/C]TAC | | | | | |
| AAATGTATGTTAAACAAGTGCATAGGGA_NODE_1_length_ | | TAGAGAAGTCGTGGCAAAGGACTTGGGATGGAATCGGTGTTG | | | | | |
| 580_cov_96.036209 | | ATGATGTTAGTGTTCGCCATTGAAAACCACAAAAT | | | | | |
| RAD_kmer_0001659_1_TGCAGGTCTTTTCATTTCTTTAGTAT | 444 | ATTGAAGTGAGGCTTAGTAAACCTTCATCTTCATGAGCTCCG | — | Y | T | C | GYSNP0485 |
| TATCCCCAGGTTCGCAATTCATCCCTAGTATATTTATAGCC | | ATAATCAGCATCAGTTCCTTGACATTTGATAAAATAGG[T/C]A | | | | | |
| ATTAATAGAGATATATTGTGAAAT_NODE_1_length_551_cov_ | | GGGAATTGAGCACCAACAATCGCTACAACAAGAAAGAAACA | | | | | |
| l09.938293 | | ATTTCACAATATATCTCTATTAATGGCTATAAATATAC | | | | | |
| RAD_kmer_0001660_1_TGCAGGGTGAAGGGAGATAGTCAG | 176 | TCGTTTCGTTAATTGTTTGTAATTACCAGTTAATCGAATTAAG | K | G | K | G | GYSNP0486 |
| TTATAATCTCAGAGGCTAAGTTGAAGGATATGATATTGGG | | TGGATTTCATGTTCGTTGCCTAAGGTGTAACTCTTAC[T/G]GGT | | | | | |
| GAATTTGCTTGTTATGGTATATATGGT_NODE_1_length_497_ | | CACATCTCAGTCAATAGCGTTAAGTTATCGAAACCGTACACA | | | | | |
| cov_66.388329 | | TAAATCCAACATATATATACTTTATAAATAAAAGA | | | | | |
| RAD_kmer_0001660_1_TGCAGGGTGAAGGGAGATAGTCAG | 355 | TAATTAGAGATTTAGACACAATTCAAAAGGTCAATCTAAATC | M | C | M | C | GYSNP0487 |
| TTATAATCTCAGAGGCTAAGTTGAAGGATATGATATTGGG | | ACGTAATCCAACATGTTTTATGATTGTGTTGTCGAACC[C/A]C | | | | | |
| GAATTTGCTTGTTATGGTATATATGGT_NODE_1_length_497_ | | TTACTTTAATTGTTTTTATGGGTAGAATTTGTCTTGTGATTTTC | | | | | |
| cov_66.38S329 | | TAACTGATGATTTTAGCTATAAATTTAGTCTCTTT | | | | | |
| RAD_kmer_0001661_1_TGCAGGGAAATTCCTCTCTOGATG | 381 | TTCTCTAACGCTCTACTGTAAAAATCTATTTGGTTTAATATGC | C | Y | C | T | GYSNP0488 |
| TATGCGAACTGAAGATAAAATCGAGTGTTTCCAGTATGCT | | ATATTATGTTGTTAAGGATTTACGTTTTGTTTTCTTA[C/T]TGT | | | | | |
| TTATCGTCTCCTGGTTTGGAAAGGGA_NODE_1_length_529_ | | TTGCAGATCAAGGAGTCATTTCCATACTCAATAGTAATTCCTT | | | | | |
| cov_136.451797 | | CACCACAAGGAGAATGTATGGTCTTTCCAAAAGG | | | | | |
| RAD_kmer_0001668_1_TGCAGGGGTCAAATGAGTGCTGTTG | 150 | GTTCCCTCAACATGCCTCTGCCAGCTCATCAGCTGGCCCGTTA | C | M | C | M | GYSNP0489 |
| ACCATGTTTGACTGATAACCCTGTTGGCCAGGTAGGATTG | | TTAACCCAACCTCAGTTTATTGATAAACAGCAAGTTC[C/A]TT | | | | | |
| GTGGCTGACCATGAAATGTATTGTTG_NODE_1_length_545_ | | CCCAAAATGAATATAGTGCTGTTCCCAGTGGGGAGAAGGTGC | | | | | |
| cov_136.957794 | | TGCAAGTGGATTATAGTGGGGTCCCACATGATGACT | | | | | |
| RAD_kmer_0001670_1_TGCAGGACTCCCGTCCGCTGCCGTG | 357 | GATAGAATTGATTGATTGTATTCCATGCAATAGGGTTACAAT | T | — | Y | C | GYSNP0490 |
| CCGCCGCTGCGCTACCGCATTGGATGTCTCTTGCATGAGT | | ATATAGAGATAAACCGGGCCGTGGGCTCGATATTAAAA[C/T] | | | | | |
| ACGGGATAAATTAAAAGTTGTGGACT_NODE_1_length_415_ | | CCTAAATACTATATTCTAATACTCCCCCGCAGTCATAGCGGTA | | | | | |
| cov_38.060242 | | GAGAATCAAACGATCAGACTGGAAAACAATAAAAAAA | | | | | |
| RAD_kmer_0001673_1_TGCAGGTTTCTACACCGCCGAGAAG | 315 | CATCACCCAATGCGAATTCATCAAGGCATCATGCGCATCGCG | C | M | C | M | GYSNP0491 |
| GTTTTAATCGCTCTCTGAATACAATTTAGGTTAATTTTATA | | CATTATACGCTCCTGATGTACGCACTTTAAAACCAAGG[C/A]G | | | | | |
| ATTATTATAGATTACTACTTTACTA_NODE_1_length_582_cov_ | | CTTAAGGCGAGGGCTCGAGATAAGCAAAGCGCATAGCAT | | | | | |
| 163.302399 | | AAACATACCATTTTTACAATTTTAGACATGTTATAATC | | | | | |
| RAD_kmer_0001673_1_TGCAGGTTTCTACACCGCCGAGAAG | 481 | TAGGCAGATTAGGCTACTAGCAAAAGAATCTAAACAACATAC | T | C | C | C | GYSNP0492 |
| GTTTTAATCGCTCTCTGAATACAATTTAGGTTAATTTTATA | | GAACAGAGAGCACTAAAATATTAATATTAACGAGCTAT[C/T] | | | | | |
| ATTATTATAGATTACTACTTTACTA_NODE_1_length_582_cov_ | | GGTTCGGTTCGGCTCACAAACAACCCTAGCCATGCCTAGTAA | | | | | |
| 163.302399 | | AGTAGTAATCTATAATAATTATAAAATTAACCTAAATT | | | | | |
| RAD_kmer_0001675_1_TGCAGGACGAGTTTAGCTTCATCCA | 193 | AACCATGCAGGAGCTGTTCTTTACATCAAAAAAGAATTCCAT | T | C | C | C | GYSNP0493 |
| TCTTACCTTTACTGACCAACCACCTCGGAGATTCAGGCAA | | CTAGAAACACAACCGACAATCGAAGGGCTAATCGTGTC[C/T] | | | | | |
| GAAAAACACCGTGAGTACAAAGTACG_NODE_1_length_634_ | | ATGTCACTTATCGGTGCTACAGTGATCACCACGTTCTCGGGAT | | | | | |
| cov_121.167191 | | CCGTGTCAGACGCTATTGGAAGACGTCCAATGCTCGT | | | | | |
| RAD_kmer_0001679_1_TGCAGGCCAAGAAAGTCTGAGATG | 223 | AACATTTCTGGGGTGTGAGTTTGGAGGTCGATTTCTGATAGA | G | A | G | — | GYSNP0494 |
| ACACCACCCAACCGTAGACGTTCTGATTTGTTTGACTCGA | | CGCCTTCTCATTAATTTGTCATACCATGTATCGGAATC[G/A]G | | | | | |
| TAAAATCCCTAAGATTATCATCTCTT_NODE_1_length_553_ | | GTTTTTTCCCATTTGCAACGAGCCCATCGCCATTTGTGGCTTC | | | | | |
| cov_77.719711 | | ACCTTGTTCTGACATCCCGTTCGCTAAGCTTCTACA | | | | | |
| RAD_kmer_0001684_1_TGCAGGATACCCGACTTGCATAC | 189 | AGTTATAAATAAGGTTTGTTGCTAAATAGGTAGAGTTGTAAT | R | G | A | G | GYSNP0495 |
| GACTTTTGCTACACTATACTTCATTGTCATATTCGATGATT | | TTGGTTACTAATTTGAGCATGTATTCAAGTTGGTTGCT[A/G]A | | | | | |
| CTGAAAATGAACTTGCTATACTAGA_NODE_1_length_514_cov_ | | AACCGTGGGAAAATGTGAAGCTTGTGTCTACTTAGCATAGTC | | | | | |
| 130.268478 | | TGCCCTCTTTATTTTCCACCAAGTATAGGATGTTGGT | | | | | |
| RAD_kmer_0001684_1_TGCAGGATACCCGACTTGCATACAA | 338 | TAGGATGTTGGTTTTTTGACCCACATACAGTTTCTAACTCAAA | R | G | R | G | GYSNP0496 |
| GACTTTTGCTACACTATACTTCATTGTCATATTCGATGATT | | AAAGATTAGAGCTAGTTCACGTGTTATAGATTAACAC[A/G]CT | | | | | |
| CTGAAAATGAACTTGCTATACTAGA_NODE_1_length_514_cov_ | | AACATGTTGGTTAATGGCCCATTGGTCGTTAGGCTGCATAAA | | | | | |
| 130.268478 | | GAAAATTATAATTTAAACAAAGAGTAATATAATAAA | | | | | |
| RAD_kmer_0001687_1_TGCAGGCTCTTTGTACTCAAACAGC | 126 | AATCCAGAAAATGTACCATTTCCAACTAATACATTGGCACGT | R | A | G | A | GYSNP0497 |
| TATTATTGCATTGAAAAGGGGATGTAATTGGGAGAAACTT | | AAAAGATTTGATTCCACATCACTTGCAACGTCAGCTTC[G/A]T | | | | | |
| CTCAATCTGCTGAAGAAAAAGGCTG_NODE_1_length_518_ | | TAAGCAAATCCTGAATTCGGGACCACCAACAGTCACTGTTCT | | | | | |
| cov_148.517380 | | TCTTTCTGTCCAAGACAGAAACAATTACAGTCTTGAT | | | | | |
| RAD_kmer_0001688_1_TGCAGGGTACGTGTGAAGAAACCA | 274 | ATAACTGCTTTGCAACCTCTTCCCGACAGACGACATGTTTGGT | T | Y | Y | T | GYSNP0498 |
| GAGTCCCCGTGCACAATCGATAGCGATTTGAAGACGTTGT | | TGGACTTAAGACTGAACATACACACATGAATATACAT[C/T]TT | | | | | |
| ATCCATGTTATAACTTTATCTTTACTA_NODE_1_length_606_ | | AGTTGATCTCTTGCGTTTTGAATTTGAATGGTTTTTTTATTTGA | | | | | |
| cov_132.014847 | | ACAGGGAAGGTGTATAAAGGCGTACTCGCGAATG | | | | | |
| RAD_kmer_0001690_1_TGCAGGACTTATTGTGTATAGCAGC | 162 | GACATGGTGTTTGATCTTGCCTGAAAGTTAGTTAGATATACTT | C | C | A | C | GYSNP0499 |
| AGATCAATCTTTACGGTTCCCTTCCTCCGCTTTTCTATTGA | | TTTTTAGCATATGATATAATACATTATATAATTATAA[A/C]AT | | | | | |
| GGGCATTTTCCGTTATTGAAGGTGA_NODE_3_length_518_cov_ | | CAGGTATTAAATTTTGTTGAATGCACGTTACATCATCAACTTG | | | | | |
| 59.687260 | | TTTTTGCATTTCAGCAACAAGTTGTGCTCCACTGG | | | | | |
| RAD_kmer_0001692_1_TGCAGGAAATGGGCGGTAGATGAA | 521 | TCGGTTTATCTATTCTTTTATATACTTTCCTGTTGTTTCTTTAT | S | C | S | S | GYSNP0500 |
| AGATTATGCACATTCTAGCGCGTGATATCGACCCAAAGGT | | GGTAAGATGAGATCAAGATTCATATGGTCTTACTAT[G/C]TTT | | | | | |
| TATTGATGTTTGGTTTATTAACAACTA_NODE_1_length_621_ | | TCGTATAGAAATGCGGTCATGTTATAGATATATAGTTGTTAAT | | | | | |
| cov_123.280190 | | AAACCAAACATCAATAACCTTTGGGTCGATATCA | | | | | |
| RAD_kmer_0001715_1_TGCAGGTTTTGTAACGGGGAAGAAA | 374 | GTTGTAATTAGTGCAACATGAGATGCACTCTATTTTCAAAAA | Y | — | C | C | GYSNP0501 |
| AGAAGACGTTGTGGAGGGGCAGCCGGACAGTGGTGACTA | | ATATTTACTACTACTACTATACAATCTGAGTACATATC[C/T]AT | | | | | |
| GTAGTAGCGGAGGGTGGCTACGAGGCG_NODE_1_length_515_ | | CATCTCGTATGTATCCGTCTCTCACCAAACGCACCATCACCTA | | | | | |
| cov_113.506798 | | CCAGTGTCCAGTGGCCTGGTCCGCTTGGTCCCGCC | | | | | |
| RAD_kmer_0001719_1_TGCAGGTTGCTAGGCTTCTGATGGA | 114 | AAAAACTAACAAACTCAGAAATACTGGGAAGACACTGTACA | A | G | R | G | GYSNP0502 |
| CACCTGTGTGGCACAGTTTTATCCAACTTTATTTGTTCTAG | | TGAAGTCATGAACAAAGAAAAGATCAAGATCTAAGCAAC[G/ | | | | | |
| CTACTGATATAATGGACATGTTAGG_NODE_1_length_580_cov_ | | A]ACTTCACTAAGTTTAATTGTTACAAACATATAATATACCGA | | | | | |
| 179.853455 | | ATGAAAAATAAGGATAAGGGATTCACATGCGTGGAAGAA | | | | | |
| RAD_kmer_0001719_1_TGCAGGTTGCTAGGCTTCTGATGGA | 396 | TACCAATTGGGCAATTGTCCATGTAATTCTTTCTCCGTCATAA | G | R | G | R | GYSNP0503 |
| CACCTGTGTGGCACAGTTTTATCCAACTTTATTTGTTCTAG | | ACAGTGGTATTTAGTAGTGAAATAATTTCATGATTCT[G/A]TT | | | | | |
| CTACTGATATAATGGACATGTTAGG_NODE_1_length_580_cov_ | | TGCATAGTAAGGTGTGTTTAGAGGCTTCAGACGAAATAAAAA | | | | | |
| 179.853455 | | CCTGATAAAGTGCAAATCTTGGTCCCATCATCTGCA | | | | | |
| RAD_kmer_0001722_1_TGCAGGATTGGAAGGTCAAAACGCT | 246 | GGTTGGAAAAGGACACAGCTGCTACTAAACTCCTCTCCAAAT | A | T | T | T | GYSNP0504 |
| CCGGTGAAGCATTAATCATTTGGCTATGGATTGTCGTTTA | | CCGCATCCCTACTGTGGGGCTAAACAAATTCATTAACG[T/A]G | | | | | |
| TTTTGTTTGTCGGGTATTATCTAGTT_NODE_1_length_426_cov_ | | GTAATCTTTATAGACCCAAACAATTGAAAATGACATTCATTTT | | | | | |
| 93.732391 | | GAGAAACAGAAATGTAACAATAAATGTTCATTTTTT | | | | | |
| RAD_kmer_0001723_1_TGCAGGCCTTCATGTTGGGCTATGT | 480 | TCCTTTTGACGTGGTGAAGACAAGATTACAGACACAGGTATG | G | G | T | G | GYSNP0505 |
| CTCTAAAAGTATTATACACACCATTATATCGGTCTGCACA | | TATTTGTTCAAGTCATTATTGCATTTTTGTTTTGTACG[T/G]GA | | | | | |
| TCCAGGTATCTACACAAAATCCCATT_NODE_1_length_598_ | | CTCAAGGAGCGCTTTTCTTTGCATCGTGTTCCTCACATGAATG | | | | | |
| cov_102.698997 | | AATAAAAAAATGGGATTTTGTGTAGATACCTGGAT | | | | | |
| RAD_kmer_0001726_1_TGCAGGTATATAGCGAAATGGTGGT | 86 | AGAATTGTGAACTTAATGAAGAGCGAAAAGATGTTCGAAT | T | C | T | Y | GYSNP0506 |
| TTTTTTTAAACAAAGTAAATGATAATGGAATTAAGAAAGA | | CGCAGGGAGGCCCCGATTATTCTTTCTCAGGTATAAGCA[T/C]T | | | | | |
| ACATTAATTAAATTACGTACCAGCCA_NODE_1_length_625_ | | TTTATACAATATTCAATCTAACTTTTGTATTTAATTACTGACT | | | | | |
| cov_79.670403 | | GGTGATGATTAATTTAAATTTATGACATGTTATGAT | | | | | |
| RAD_kmer_0001737_1_TGCAGGGAAGCTTATGTGTGACAGG | 122 | AAAAAAGCACATTAGAAAATTAAATATACCTATTTTACAAT | W | T | T | — | GYSNP0507 |
| ATATGAAATCTATATATTTATGGATATTATTATATATATGT | | AAATTTTTATTTAATTAAAAAACAAAAAGTTATAAAAA[T/A]T | | | | | |
| AGTGTTTGATGTGTATATTTTT_NODE_1_length_480_cov_ | | TTTTTTAACTATTGTAACGATTGCTTTGTTAATCTTTTTAAGAC | | | | | |
| 72.418747 | | TAGAGCTGGTAACAAAATTACTTAGAATTCAAATT | | | | | |
| RAD_kmer_0001744_1_TGCAGGAAACCGCCACAATTACCTG | 358 | ATGACATACAATGCTAGGAACTTGCTCTCAAAGCCGCTAATC | R | A | R | A | GYSNP0508 |
| CTACAACACGAACCAGTTCACTTCCATCTGAACCGTTGGC | | GCGCCACAAAATCATCATAATCAGGAAGTTTAGGATGC[A/G] | | | | | |
| TGCAGGGTTGGCGGTAGATGAGAGAA_NODE_1_length_483_ | | CATTACCGCGCCGACCCAAACCCGACTCCGCCTGATAAGTAG | | | | | |
| cov_83.385094 | | CTGCCCTTGCAAGCCCTTTTCTCTCATCTACCGCCAAC | | | | | |
| RAD_kmer_0001749_1_TGCAGGGAGAAGTGTCTAGAAGAT | 104 | TCCTATTTTGACTTTTAAAGTCTTTCACTCGCAACTTTGACCG | A | T | A | — | GYSNP0509 |
| AACTGCCCGATATGCCATGAATACATCTTCACATCGAAGG | | TAAATTATTTTGATTGTGTTATATAATATTTGATGTA[A/T]AAT | | | | | |
| ATCCTGTGAAGGCCCTTCGGTGTGGTC_NODE_1_length_255_ | | ATATGGATAGATTGAGTTTTAGATGTATTTTTCATTGATATAA | | | | | |
| cov_9.403921 | | CTTTCATTGGATATTATATCATACAAACAAAAAT | | | | | |
| RAD_kmer_0001753_1_TGCAGGGAATTTTCTTTTATCATTCG | 268 | TTAAAAAAATTGACTGTTTTGTATCATACTTATAAATTATCAA | T | — | T | A | GYSNP0510 |
| ATTTCATTTATGACTTATTTAATGTTTTTTTATGTTCAAAC | | CTTACAAAAAGGATTAAAAGAGTCGGAATGCAGAAAA[T/A]T | | | | | |
| CACGGTCGAGATGAGCCAGCGAAGC_NODE_1_length_586_ | | AGACATACCCCTTAGTGAGGTCTCATTGATCCAGAGGCAACT | | | | | |
| cov_105.817406 | | CATCAGTGGCGGAGCTTGAAAAATTTTTCCAGGGGGC | | | | | |
| RAD_kmer_0001754_1_TGCAGGATGGACTGTGGAAGATAA | 115 | TTTAACCATTAGTCCCATTTCCCATTTAAATCCTTTTCTTCTTT | C | C | Y | T | GYSNP0511 |
| GTTTCGGAAAGGTGGTAACAAAACTGGAATGAAATATAA | | GAACCATTTAAGATAAAATCTACCCTGTAAATGGGT[C/T]AGC | | | | | |
| GGTAAGTATAAAAAAGTTTTACCAATAT_NODE_2_length_360_ | | ATTGCCACCTCTAGCTAACAATATTATAGAAAACACATTATA | | | | | |
| cov_26.025000 | | TGATTAAATAAATACCCAAGATGAAATCGGCAAAG | | | | | |
| RAD_kmer_0001780_1_TGCAGGCCATCAGGAACTAAAGTG | 114 | TTTTCCAAGCCCCACCTAATAAAATAATAAACCAGACCCCCC | C | T | Y | Y | GYSNP0512 |
| GTTATAAAGAAAATACTAGAAAACAAGATGGGTCTGGTT | | TCCCTCTCTTCCCCTATATTAACCAATACAACAAAAAT[C/T]A | | | | | |
| TGGTCTAGAGTTGAAAAAAAGCAAGAAAT_NODE_1_length_ | | CTAAACAACCCTCTGTACAACTCCATATGCACAATGTACA | | | | | |
| 490_cov_165.771423 | | CATCACTGAATAACCATTCACCTGGATTTGGTTATTG | | | | | |
| RAD_kmer_0001781_1_TGCAGGATTGAGCAAATATAGCGA | 457 | TCGTATGAACACGATGCTAGAAGTGAGCCAAACTTGGGGTGA | C | T | C | C | GYSNP0513 |
| GGTAACATACATTAACAGCAAATCATGCCAGGGCAGAAG | | GCCCAAGTAACCTGCCAAACAGGACCGTTATGACCGGT[C/T] | | | | | |
| ATTGAAACAGGTCACTCAGACATTATCC_NODE_1_length_622_ | | AAAGTAGCAAGGGGTACTGAGCTTAGATTGTTTATCTTGATA | | | | | |
| cov_121.982315 | | GTCGCATCAGAGGAGGCAGATGCCACCCGCTTTCCATA | | | | | |
| RAD_kmer_0001788_1_TGCAGGCTAAAACAAGAAAATTAC | 286 | AGAGTAATGTCCTTATACAGGTTGATAACTTGGACATTTTTTT | A | R | R | G | GYSNP0514 |
| ATGAATAAAAGAGAAGATTTCAACCACTCTACCCACTTTA | | AAGGGTTTACTAAAATCCTAAATTTACCCCTTCGAAT[A/G]CT | | | | | |
| CTTTACACTTATTCTTACTCAAATACC_NODE_1_length_620_ | | AAACATACGCATCTATTTAGCAAAAGTAAAAAAGATAACTAA | | | | | |
| cov_123.345161 | | CTCATTTATTTATCAGGGGTTGCCGATTCGAGATAC | | | | | |
| RAD_kmer_0001791_1_TGCAGGTGTTAAATGGGTCATGTTC | 322 | GGCGTGAATTTTTTAACACGACACGAATCTAACATGAAATTC | M | M | A | M | GYSNP0515 |
| ACATGTTGAACCTGAAAATCGTGTCAGACACATAAACCCA | | GTGGGCTTGGGCTTTGTCTAAACAGGTTCGGGTCCTAA[A/C]T | | | | | |
| AACACGACACGAGTTTTAGTGAACTG_NODE_1_length_580_ | | GGGCCGTTTTCAGGTCAAGCTGGCAGATTCACGTGTGGATTA | | | | | |
| cov_118.608620 | | CTTAATCCAATTAAATTTTATACTTATTTTCCAGAAT | | | | | |
| RAD_kmer_0001795_1_TGCAGGTACATAAAGGAAACAAAA | 382 | TATTGACAAATAAATATGAGTTATCGTTCATGTGTAATTCTTT | A | A | W | T | GYSNP0516 |
| TTAGTCTTTGACCCATTATATGCTCCTTTACTACCATAATC | | TTTCTTTCTTCATGTCTGTCTCTCACCTGAAGCATTT[A/T]TGG | | | | | |
| AATAGTAAGCTTGATAATTAAAACAA_NODE_1_length_577_ | | TGCATTTTAAATTTCTTTTGTTTAATTTTTTTAGTCACATTACC | | | | | |
| cov_101.636047 | | TAGATGTTTAAATGCTAAATTTTGTTTTACCTG | | | | | |
| RAD_kmer_0001795_1_TGCAGGTACATAAAGGAAACAAAA | 476 | ATTTCTTTTGTTTAATTTTTTTAGTCACATTACCTAGATGTTTA | A | A | W | T | GYSNP0517 |
| TTAGTCTTTGACCCATTATATGCTCCTTTACTACCATAATC | | AATGCTAAATTTTGTTTTACCTGGTTGTATGAAATA[A/T]GGA | | | | | |
| AATAGTAAGCTTGATAATTAAAACAA_NODE_1_length_577_ | | ATTTAGTTCTGCTGAAGATGCACATGTTGGTGATTGTTTTAAT | | | | | |
| cov_101.636047 | | TATCAAGCTTACTATTGATTATGGTAGTAAAGGA | | | | | |
| RAD_kmer_0001796_1_TGCAGGTCGCTGTTATTAAATCTGA | 332 | TAAGGTGGTTGAGACACTTTGTTTTTCCTAGGATTGAATCACA | A | T | T | T | GYSNP0518 |
| CCCTCTTGAAAGTCTTAAACGTGAACAGTGGGTCGCCATT | | CGCATCGAGACTCATGTACATACAAACATAGTAACAC[T/A]TT | | | | | |
| ACTATCCCCAACCCCCCAGCCGGTCG_NODE_2_length_479_ | | CAAAACTAATAAAATCACCATTATAAATGGCGACCCCTCCCA | | | | | |
| cov_31.256784 | | CTTCTCTTACACATTTGAGCTTTTCAGTACGAGGTT | | | | | |
| RAD_kmer_0001808_1_TGCAGGTTGATAAGTATGAATCTTA | 114 | TCTATCTCTAAAATCAAAATATTAACTTTTTTTAAAAGGCTTA | C | Y | T | Y | GYSNP0519 |
| AATAGTCGCATTCGATCACCTAAAAATTTATAGTCAATAC | | AACGGGTCAACATTTCGACCCACTTTTTAAGACGATT[T/C]GT | | | | | |
| GTATGAGAGGGTTTGTATGTCAGATG_NODE_2_length_340_ | | TCGGTTGACCCATAATATGAGATTACTCTATGTATTTTACACC | | | | | |
| cov_211.202942 | | ATTTTTAACCCTATCCACCTTTCTGAAAATTTCCC | | | | | |
| RAD_kmer_0001809_1_TGCAGGGAAAGATGAAAACAAAAT | 302 | TTGGGCTCGTACAAGTTATTACTGCTAATTGGGCTGCTGTTTA | T | G | G | G | GYSNP0520 |
| TTACCCTCAACTGTTGCCAACAAGTTATGATAACAAACAA | | TTGGTCTATTAGATAGATGTATTTGGAATTGGCTACT[G/T]GA | | | | | |
| AGGTGATATATGAAAACTTTTGTAAGA_NODE_1_length_611_ | | TGAATTGAATTTCAGGTGCTTGTTCCAATTGCTACTGGAACGG | | | | | |
| cov_107.509003 | | AGCCGATGGAGGCTGTGATTGTTATTGACGTGCTC | | | | | |
| RAD_kmer_0001813_1_TGCAGGTATGCATTTGTGTCATTCA | 571 | TGCAATGTGATATGGCAGTTCCAATCGCTTCTTCGCCAATATT | G | S | S | G | GYSNP0521 |
| AGTTATACTGAAATGTCTAAAATGCCCCCCAATTGTACAA | | GCAAGACTATGTAACACAAAATAAGTAAATAAAAACC[G/C]C | | | | | |
| ACTAGCTGAATGTGGATATAAAATGT_NODE_1_length_673_ | | CTCGTAAATTATCTCTTTTTTCATGACAGAAGATATACATTTT | | | | | |
| cov_102.707283 | | ATATCCACATTCAGCTAGTTTGTACAATTGGGGGGC | | | | | |
| RAD_kmer_0001817_1_TGCAGGGTACAGAAGAAACGTGGG | 353 | CGAATTTATTCAAATCACCTGAGGCATCTGCCACGCGTCAGG | T | C | C | C | GYSNP0522 |
| CATCTGCCTTATGAACTCCTCCAAGAAGGTTACTTTCTTGT | | AATATCAAGCCTTGAAGCTGAAAAAATCTTCAAGAATT[C/T]A | | | | | |
| TCTCTGTTTAAAATCTTGAATGCCCA_NODE_1_length_465_ | | AACCCAAATCAGCACAAAACTATAACCAGTTTAAACAAACTT | | | | | |
| cov_153.823654 | | AATATGGGCATTCAAGATTTTAAACAGAGAACAAGAA | | | | | |
| RAD_kmer_0001820_1_TGCAGGCAAGACATGGAACACGTG | 154 | AAACTACTAAACACTGTTGGATAGAACCTGTCTTTCAAAAGT | G | G | K | — | GYSNP0523 |
| CACCTACCAAGAAAAGAATGAGAGATTTGGAAATTTTATC | | AAATCATTTTGGAGACGGATGCCCCTCAAAAGCAGGTT[T/G]A | | | | | |
| CGTTCAGTTAGAATCCCTTTTGCAAAA_NODE_1_length_628_ | | TGTTGTATTTCCTTTTCTTATCTTAATAAAATATACTCTGGGTC | | | | | |
| cov_71.019112 | | CTATCACATATACTAAAATCAAGGAGAGAGGGGAT | | | | | |
| RAD_kmer_0001820_1_TGCAGGCAAGACATGGAACACGTG | 252 | CTTATCTTAATAAAATATACTCTGGGTCCTATCACATATACTA | C | C | T | — | GYSNP0524 |
| CACCTACCAAGAAAAGAATGAGAGATTTGGAAATTTTATC | | AAATCAAGGAGAGAGGGGATATTTGTACAAACAATTA[T/C]A | | | | | |
| CGTTCAGTTAGAATCCCTTTTGCAAAA_NODE_1_length_628_ | | CACTTTTAAAATATGAACAAGAAGCTTGAAACTAGTATACGA | | | | | |
| cov_71.019112 | | TATAATAAAAACCGAACCACATTCAGAAATTTATATG | | | | | |
| RAD_kmer_0001821_1_TGCAGGAGGAGGAGGGATATACAG | 396 | ATTTAATTACACAATTAAACATTCTCAATTATTTAGGAGAGCT | K | T | K | T | GYSNP0525 |
| AAGGTGGTTTTGGAGAGAAGATTGAAGAATTGGTCGTTCA | | AGAATAAGCAATTATAATTTGTTAGTTATGCTAATGC[G/T]TA | | | | | |
| TTTTCAAGTAGTGTTATGTGTTTGTCT_NODE_1_length_647_ | | GGTGAACCTAATCTAATCATATTAATTAAACTTTGCAATTTCT | | | | | |
| cov_85.533234 | | ATATACCTAATTTGTTACAAAGTTATTTCTAAACT | | | | | |
| RAD_kmer_0001828_1_TGCAGGCTTAGTTTTTATAACTACA | 98 | AAGAATATCGTGGATTCTATTAGGGTTGTTAGTTCTTACTCCT | G | — | K | G | GYSNP0526 |
| AATTCAAGGATCAAATAGTTGGTTGAAATTGACAAAAAT | | CCATTTCCAAGGGCAATTGCCATAATAGCAACAACTT[G/T]CA | | | | | |
| GTTGTTGTAGGATCTAGACAAGTTTAG_NODE_1_length_610_ | | TATGAAGCCTAAAAACTTAAAGAATTTTCTCTCCTGTAAATG | | | | | |
| cov_141.614761 | | CCACATCATAAATTCATCACAAATTGAAAATTATCT | | | | | |
| RAD_kmer_0001828_1_TGCAGGCTTAGTTTTTATAACTACA | 178 | GCATATGAAGCCTAAAAACTTAAAGAATTTTCTCTCCTGTAA | C | — | Y | C | GYSNP0527 |
| AATTCAAGGATCAAATAGTTGGTTGAAATTGACAAAAAT | | ATGCCACATCATAAATTCATCACAAATTGAAAATTATC[T/C]A | | | | | |
| GTTGTTGTAGGATCTAGACAAGTTTAG_NODE_1_length_610_ | | AAACAACAAAAAATCTCTACACTCAACTGAGATTTCTTTACA | | | | | |
| cov_141.614761 | | AATTTACACAACAAACGAAAAGAACTATTTACATACT | | | | | |
| RAD_kmer_0001828_1_TGCAGGCTTAGTTTTTATAACTACA | 291 | ATTTCTTTACAAATTTACACAACAAACGAAAAGAACTATTTA | Y | — | C | C | GYSNP0528 |
| AATTCAAGGATCAAATAGTTGGTTGAAATTGACAAAAAT | | CATACTACTCAAATTCAATCGGCCTAATCATTTAGGAA[C/T]T | | | | | |
| GTTGTTGTAGGATCTAGACAAGTTTAG_NODE_1_length_610_ | | TTCATGTTTAATTAGATATACGGTGGCCATGGTGTTAACTTGT | | | | | |
| cov_141.614761 | | CCAAACTGGCCCGTTTTGACACATAGCCAATCGAGC | | | | | |
| RAD_kmer_0001839_1_TGCAGGTTTTTGTATTTTTAGCTGCT | 160 | TAAAACATCTGGTCTAAAGAAAACAAGAAAAACAGTTGTCCT | G | G | R | R | GYSNP0529 |
| GAATATAGTACTCCAAAAAATGCCCTGAATCAGGTATGAT | | GTTTAAAGTATAGAAAAGCTATTGTCAAATCATGTGCT[G/A]C | | | | | |
| TAACGTCTTTCATCCCGTTCCCAAA_NODE_1_length_595_cov_ | | CCATGTTATCATCTGTATGAGGAGTATGACTCTGGCAAGCGA | | | | | |
| 148.584869 | | AAACCAGGAAGAACTAGTTTGTCTGCAAACATCTTAC | | | | | |
| RAD_kmer_0001840_1_TGCAGGTTAGTATGTTACGATTTAT | 148 | AAATAACATCTTTCAAAAACAAAAGCTAAGTGGGCTGGGTGA | G | G | A | G | GYSNP0530 |
| TTTTCACAACAGTAACTTTACCTTCGAATTATAGTAAGAA | | TATGTACCTCCTAGCGACGTAGTCATGCATGGATCCAC[A/G]A | | | | | |
| CTATTTTATATGTTACAAATATTGCA_NODE_1_length_622_ | | TTTCAATTTATTTCTTACTAGGTTATAACCCGTGAACTTCACG | | | | | |
| cov_99.475883 | | GGTTGTCCAATTTATTTATTTAATTCACATAAATTA | | | | | |
| RAD_kmer_0001846_1_TGCAGGATATTCATGTGAGAATGAG | 325 | ATAACAGCATCAGGACTTGGAATACGGAATCCGGATGAGGTT | C | C | C | G | GYSNP0531 |
| GGAAATTTGTGTTTAGAATTGTGTTAAATGGACAGCAAGG | | TACACATGGAAAAAGTCAACTTTCCTTCACCATTGGAC[C/G]A | | | | | |
| CGAATAAGAATGAAGATGAGAATGTC_NODE_1_length_431_ | | AAAAAGCTATATCAACCTCGGAGGAACATGGTGATGGCGCG | | | | | |
| cov_117.626450 | | ACATTCTCATCTTCATTCTTATTCGCCTTGCTGTCCAT | | | | | |
| RAD_kmer_0001847_1_TGCAGGTTGGGAGACTTTTGGGTCC | 172 | GGTGCTGGCAATGTTCCTGAAACCGCTAGCTCAGTCCACTCC | W | T | W | T | GYSNP0532 |
| TGGATACTGGTGAAGCCCTTCAACTATAAAATCTTTAATT | | AATGACACTGTTTAAAAAGCATAAATAATGTGTGTTGA[T/A]A | | | | | |
| AACAAATACAATTGATAAATTTCTTT_NODE_1_length_587_ | | AAAATATATAAGAATCACATTAATGACACAAGTAAAAGAAA | | | | | |
| cov_147.364563 | | AAAGATAGGATTTCAAAGGCAAAATCTTTACTTGTATC | | | | | |
| RAD_kmer_0001849_1_TGCAGGTACATGCTTACTTCATGAA | 397 | CATTAGGCCAAGGGACCCAGTGGGATGGAACAAAATTTAGC | A | C | A | A | GYSNP0533 |
| AAATCTCTTTATTTATTATGCACATTAGCAACTCCTAATTT | | CATTTAGTTTAGAGCTGCTAGTGATGCAAAAGATAAGAG[A/C] | | | | | |
| CATGATAAATAATATTCATCACTCC_NODE_1_length_591_cov_ | | TATTGATTCATGTTATGTAATCAACTATTCAATTACCATCAAC | | | | | |
| 146.766495 | | AAACTGGGTTTCTTTATAGAGACGACGAGTGTGGCAA | | | | | |
| RAD_kmer_0001850_1_TGCAGGAAAAGATTACTCTGTTCAG | 109 | CAAAAAAGGCCTAATACATATACATGAACCCAAACCCTTAAT | A | C | A | A | GYSNP0534 |
| AAGCTGATTCTTGGGACCCACACATCGGAAAACGAGCCT | | TCAGAAAAACATAATAAAAACTGAACCCTATAAAACAC[A/C] | | | | | |
| AATTATTTAATGCTGGCTCAGGTTCAG_NODE_5_length_318_ | | AAAAAGAACAATAATGTTCTTCAAAATATAAAAACACCAACT | | | | | |
| cov_15.776730 | | CAACTATCAGTTTCAATCTCCACAACTACACCTACATG | | | | | |
| RAD_kmer_0001852_1_TGCAGGAAAAGATTACTCCGTTCAG | 117 | AAAAAGCGAGACGTGTCATATGGACCGTATAAGTAGGTGAA | W | — | T | T | GYSNP0535 |
| AAGCTGATTCTTGGGACCCACACATCGGATGATGAGCCTA | | AACGGTCAACTGAGTCGTATGACAGAGCAAAAATGATCA[T/A] | | | | | |
| ATTATCTTATGTTAGCTCAGGTTCAG_NODE_1_length_596_ | | ATGGGCCATATGACTTTGGCGGTGGTCACATCACTCAATCAA | | | | | |
| cov_82.293625 | | ATGCATCCTACGATCATAAGGGCCATGTGGCTTCGCCC | | | | | |
| RAD_kmer_0001855_1_TGCAGGTACATATTCTTATTACCCA | 353 | GCCATGCTAGAGAATTAGTGACTGGTGCCTGGTGGTGGCTTTT | C | A | M | C | GYSNP0536 |
| TCGAGCAACTACTGTGTATATTTAAAATACAGAAGAAAGG | | TGCATAGCTTTTGTATTTTCTTAACTTAACTGGAGTG[C/A]TTT | | | | | |
| GCTCGAGAAATGAGAAAATAAATATA_NODE_1_length_592_ | | ATTGAGGTACTCAGAAACAAGTGCACCATTATCATGGTCAAC | | | | | |
| cov_119.537163 | | AAGAATGAGGATTGCGCTAGGGGCAGCAAAAGGCC | | | | | |
| RAD_kmer_0001857_1_TGCAGGTATATAAATAATCAGTTGT | 271 | CAATCTGGCATCTGCCAATAGAAACCATCCGTGTTGCACAGT | C | Y | Y | Y | GYSNP0537 |
| TAGTCCAAAGTGAGACCCTTGTTTTTGGAAACCCAGTATT | | TGTGGCAGTACTTGAAGAAACCTATTTTTCAGCATGTG[T/C]T | | | | | |
| GCAAGTGGTTAGACACAAAGTGTGAT_NODE_1_length_605_ | | TGCCAACAAATTCCAACTCAAAACTTAATTTCAAACATTTAG | | | | | |
| cov_141.791733 | | ATCAGTTAGTTTGCTATTGCTTATTGTTACTCAGATT | | | | | |
| RAD_kmer_0001858_1_TGCAGGAAAGCTCAATTACAACACT | 531 | CTCATTGCAGATAAAAAGGTTCGAGTGGCCAAGGGATGAGCT | T | G | T | — | GYSNP0538 |
| GCGGGTTATCGATCTTCACCCGGACCTCTAACAAGTGCAT | | GTTCATATGGATAATTAGATCCCTCCCAAGATTTAACC[T/G]T | | | | | |
| CATTCCCCCTCCAAATGAAAAGGTTA_NODE_1_length_590_ | | TTCATTTGGAGGGGGAATGATGCACTTGTTAGAGGTCCGGGT | | | | | |
| cov_73.761017 | | GAAGATCGATAACCGGCAGTGTTGTAATTGAGCTTTC | | | | | |
| RAD_kmer_0001859_1_TGCAGGTTATAACTTGAACTCCATG | 157 | GAATATGAAACTTACTTCTTTTTTCAAGTTACTTGGCCCACCG | T | Y | Y | Y | GYSNP0539 |
| AATCATCACTCACAGAGACTGGTGGCTCTGAAAATTATGT | | AGGAAACACCCTTGTCTGTTCTACCACATCTTGAATA[T/C]TG | | | | | |
| ATTTTGGGCCGAGGTACACAAATGAT_NODE_1_length_438_ | | CAAATCCTTCCTGTCACCAAGTACAAGTCTTGTCTTCTGAAGT | | | | | |
| cov_28.796804 | | GCTTTGAAAAGTTCCGACTGCAACAAAGACAAAGA | | | | | |
| RAD_kmer_0001868_1_TGCAGGAAGCTGATCAAGTTGTG | 265 | TACGGAAGTAAATTTGTACCATATTTGGTTATTGAATTCGATG | A | A | A | G | GYSNP0540 |
| GTTTTCTCATATGAGAGAGGTGAAGCCTGGTATTTATGTC | | ATTTGCATATGAAGAGAAAGCATATGCTATATAAAGA[A/G]A | | | | | |
| ACATACAATGGAGACTTTTTTGATTG_NODE_1_length_613_ | | CATTAAATCTGAATATGAACCTTCAAACCATGGCTCCCTTGA | | | | | |
| cov_130.895599 | | GGTAGATAACTATCACGTTTGACCCACGCAAAACAAT | | | | | |
| RAD_kmer_0001869_1_TGCAGGTAAAACGAAGAACACAAT | 311 | TGAGTTTGTTCATAACTTATTATGATTTTAAGATTTTTTCTTTT | A | T | W | T | GYSNP0541 |
| TGATTAACAAGTCCATTTTAAATTAGTATACCCTTGTTCGT | | CTCTTTAGAGTTACAAATTTGAATTAGTAGTTGCAT[A/T]TTTG | | | | | |
| AATAACAATAGCCGTATGTACCTCAG_NODE_4_length_376_ | | GAAACCAGAGATCATTTTCATAATCCTAAGGTTGACTTCAGT | | | | | |
| cov_27.377659 | | TAAATGGCAATTTAATTAGAAATATTGTATCCAT | | | | | |
| RAD_kmer_0001870_1_TGCAGGAATAAAACTGTAGTTTCTA | 114 | AGAAGGAGGGCTTGAACCTCCGACCTTGTGGTTAACAGCCAC | T | A | T | T | GYSNP0542 |
| CACAAATATATTCCAGTCATTTGTAGGCATTTCTTTATGTA | | ACGCTCTAACCAACTGAGCTATTCCAGCTTTTTAATTT[T/A]GC | | | | | |
| TATTATAGACTCGCAAATTACATCA_NODE_1_length_548_cov_ | | AATGTTTAAACATAAATATATTCTTTTACCTAAAAAGTGAGCT | | | | | |
| 83.735405 | | ATTCCAGCTTTTCAATTTTTTTTTTTTCTCTTTTT | | | | | |
| RAD_kmer_0001870_1_TGCAGGAATAAAACTGTAGTTTCTA | 390 | ATGAACATTGAATAAATAGTTTTCTAAAATTTCTTCTTGTCAC | C | C | C | T | GYSNP0543 |
| CACAAATATATTCCAGTCATTTGTAGGCATTTCTTTATGTA | | ATCTTCAGGAAGAATGTCGAGATACCAATTTTGCAAT[C/T]TA | | | | | |
| TATTATAGACTCGCAAATTACATCA_NODE_1_length_548_cov_ | | GGGCAAGAGTTGAAGCTCGGCTGAATTATTTATGTCCTCAGT | | | | | |
| 83.735405 | | TTTTCCCAGCTCCAGGTTTTTGGTCTCCTGAACAAC | | | | | |
| RAD_kmer_0001874_1_TGCAGGACAAAACATCTCTGCCTTA | 96 | GTTTGTCCTTTCAAAAAATGATACATAATTAAGGCTTTAATTA | G | — | G | K | GYSNP0544 |
| TGAACCGGTCTACAGTGACGATCAAGCAAGGTGATTCCCT | | TATCAACAAAAGCAATGTCATTACAATTACAATCTTT[G/T]TT | | | | | |
| TTCCATTTTTGCCCTTGTCTTGATTT_NODE_1_length_514_cov_ | | TTGAACTAAACCTATTTGGGAATCTTTCAACCCGTCTGACATG | | | | | |
| 89.568092 | | TTCCCCTTTTATATAAATCTTTTGACTCGACATTG | | | | | |
| RAD_kmer_0001875_1_TGCAGGTCAAAAGGGTTATGTTATA | 227 | AGCACGAAAAGACCCCAATAAATATTCATCTATCGATGCCTA | W | T | A | T | GYSNP0545 |
| TCTCTATTGGGTCAAACAGGTAAAATAAGAATTAAAAAA | | ATACCATAGATTGTAGTCCGATACTCTCTGTAAGTACC[A/T]T | | | | | |
| AAATATTTAAAAGGAATGGGGTGAACG_NODE_1_length_363_ | | CTAAAATTTTGTTATTATTTATCAATATTATACCATGCTCACT | | | | | |
| cov_49.316803 | | ACTCACTAGAGGTGGCAAAACGGGCAGGCGAGTATT | | | | | |
| RAD_kmer_0001875_1_TGCAGGTCAAAAGGGTTATGTTATA | 300 | AAGTACCATCTAAAATTTTGTTATTATTTATCAATATTATACC | C | T | C | C | GYSNP0546 |
| TCTCTATTGGGTCAAACAGGTAAAATAAGAATTAAAAAA | | ATGCTCACTACTCACTAGAGGTGGCAACGGGCAGG[C/T]G | | | | | |
| AAATATTTAAAAGGAATGGGGTGAACG_NODE_1_length_363_ | | AGTATTTGTATAACATATCAAAAGGGTCTTGGTTTTGTATCCA | | | | | |
| cov_49.316803 | | TTATTCCTTTGGAACTGATCTAAAATACTCTTTTTT | | | | | |
| RAD_kmer_0001877_1_TGCAGGGATGGAGGGTATAGTAAA | 224 | ATCCTAACAATTAATGCACCCAAATCTAAAATGGACCCATAA | T | T | Y | Y | GYSNP0547 |
| TGCAGGAACGGTAAAAATCGTCATTCACCCGCCTGTTATA | | AATTAAATGAAAAAGGCCACCGACAAAAACATTCAATA[C/T] | | | | | |
| GGAGATAATCCTGATGAATTATGCACC_NODE_1_length_578_ | | CACCCTTTCTTTCTATAATCTCAAATATAACTGAATATTGTCA | | | | | |
| cov_152.221451 | | TTTTTTTAGTTAGAAATAGTAGCATAGTTAAAAGGTA | | | | | |
| RAD_kmer_0001877_1_TGCAGGGATGGAGGGTATAGTAAA | 351 | TTTTAGTTAGAAATAGTAGCATAGTTAAAAGGTATATACATT | M | M | C | M | GYSNP0548 |
| TGCAGGAACGGTAAAAATCGTCATTCACCCGCCTGTTATA | | GTCTAATTTGAAGGTGGACTACAGTTATTGGGAAAATA[C/A]A | | | | | |
| GGAGATAATCCTGATGAATTATGCACC_NODE_1_length_578_ | | AGTAAATAGTAAACTTGAGTTGCTTATAGATAATCAAAACAG | | | | | |
| cov_152.221451 | | TAACAAAAAACAAGACATTTGCACAAATTAACAGA | | | | | |
| RAD_kmer_0001882_1_TGCAGGCAATTAGATCCACAATCAC | 107 | GTGCATGTTTGGCTCTCCATTTTTGAGCTCCTTATAACTTATT | K | G | T | G | GYSNP0549 |
| ATCTCGACATCTTAACATCATACGTGCAAAAACAGAACCA | | GACTTAATCAAGAAGTTAATAAGGAGAAAATAATGTT[T/G]G | | | | | |
| ACAAAATGAAATTATCAAATTACATC_NODE_3_length_254_ | | GGTAATGGGTTGAAAAGGAGGTTTTTGTGGGTGGAAAAGGA | | | | | |
| cov_6.118110 | | GATAAGTTATAAGTTAAAATTTGTGACTTATTGATTAA | | | | | |
| RAD_kmer_0001902_1_TGCAGGCTAGGTTGCTTCTATATAT | 87 | AAAATAATATGATTTAATTTGGTTCAAAGAAATCCGAGGAAA | T | — | A | A | GYSNP0550 |
| TGTGTCATCCCGCTGTTATATACATCTACACCAAAAAACG | | ATACAATTCTTTTGGTTACTGATGTCATAAATCATGAT[A/T]TA | | | | | |
| TACACAGAGTAATTGCTTCAATTTAT_NODE_1_length_460_ | | ATCATCTGATATATTAATCTTGTACTCCAAGAGTCAAACAAT | | | | | |
| cov_223.436951 | | AATCCAGATTCTCATCACAAACAAATAACAAACGAT | | | | | |
| RAD_kmer_0001903_1_TGCAGGTATGACTTTATCTGCACTG | 305 | TGATTTTGGAGAAGTTCTTTGACGTGATTCTAAATCTACCAGC | C | Y | Y | T | GYSNP0551 |
| CTATAAATGTACAACTGTGGGCAGCTTGGTTGTCCTGATT | | CGTTCACCAGTAAGTCGATTTACTTGTATAACTAATT[C/T]AA | | | | | |
| TTAGTTGTACTAGTACATTAGATAGC_NODE_1_length_584_ | | TGTCCATATTAAAGGGCGTCTTTGGATGCACATTTCGAAA | | | | | |
| cov_142.339035 | | GTGATTATATGATATTGAAAAACCAGAATCATATAA | | | | | |
| RAD_kmer_0001906_1_TGCAGGGGGTAGCCCATAGGCAGA | 408 | ACTTACGCCACGTGGGCTTCCAAACGAACCTTCATCGTCGGA | C | T | C | C | GYSNP0552 |
| CGGAGGAGTAGGGACGGCGGATGATGGAGATATTGGTGT | | GATACACTCTTTTTTAACTTCACAACTGGATTCGATAC[C/T]GT | | | | | |
| TTCAGGAGCGTGGCAGAGTTAGAGACA_NODE_1_length_ | | GGCTGAAGTTATCAATCGGCCATGGAGGACCCATGCAATTC | | | | | |
| 640_cov_76.937500 | | CATCATCACACTCTCACTCAACCCCATTGGCCCTGC | | | | | |
| RAD_kmer_0001907_1_TGCAGGGCAAAGCGAGTAGAGGTT | 126 | GCACGAGGTTACGGGATTCGTGCGCCATGTCAACTCAAGTTT | C | C | A | A | GYSNP0553 |
| TGCTGATAAATGTTATTGTGACTCACTTAAAAATAAGTTT | | ATTTGGTCAACAAGATCATTTTTGTGATTTTCTTGATG[A/C]AA | | | | | |
| TTATTCGAGGTGGCAAGATGGGTGTTT_NODE_1_length_622_ | | AGAATCTAAAAACTCCCTACTTTTGAGCTGTTTTTTCACTTCT | | | | | |
| cov_108.921219 | | TCAGCAGATACTTTTGCATCTTGAAGTTCCTGTTT | | | | | |
| RAD_kmer_0001907_1_TGCAGGGCAAAGCGAGTAGAGGTT | 411 | GTCTACCAACCAAAAAAAAAAAAGACGTTACACATACTTAA | A | A | A | T | GYSNP0554 |
| TGCTGATAAATGTTATTGTGACTCACTTAAAAATAAGTTT | | AAGTAGAGGTTTCAATTTTGACCCGTTTACTTACGAATG[A/T] | | | | | |
| TTATTCGAGGTGGCAAGATGGGTGTTT_NODE_1_length_622_ | | AGTGTGATTTATCTCATACAGGTCAAATAAAATGAATTTGCT | | | | | |
| cov_108.921219 | | AATAGGTGAAACGATCAAATGGGGCAGACGGGTTAAAA | | | | | |
| RAD_kmer_0001908_1_TGCAGGGAAAGAAGGGGTGAGTCC | 329 | ATTTGACAAACTAAAAGTCAGAATTGAAGTGGATTTAGTGGA | G | C | C | C | GYSNP0555 |
| ACTCGCCACGAACATTGTGAAACTTGCCACACATGTTGAA | | AACATAAATCAACTGTAGATGAGTATCCTGAATACAGA[C/G] | | | | | |
| AAGCGGCTGACCATGGGAAAAAGAGGA_NODE_1_length_588_ | | TAAAGAACATCACATTCATGGAGACAGACGACGATGTTCACT | | | | | |
| cov_124.862244 | | ATACTGTTTTCCTATACAAAATCTCTTTTCATTT | | | | | |
| RAD_kmer_0001908_1_TGCAGGGAAAGAAGGGGTGAGTCC | 421 | ACATTCATGGAGACAGACGACGATGTTCACTATACTGTTTTC | T | C | T | Y | GYSNP0556 |
| ACTCGCCACGAACATTGTGAAACTTGCCACACATGTTGAA | | CAATATACAAAATCTCTTTTCAAATTTCAACACATGTT[T/C]A | | | | | |
| AAGCGGCTGACCATGGGAAAAAGAGGA_NODE_1_length_588_ | | AATTACTGTCGGGCATTTGCCGTGTGCAACACATCCTTTAAG | | | | | |
| cov_124.862244 | | ACAGCAGCAATTCTATCGGCCATGTGTCGTTCTAGAA | | | | | |
| RAD_kmer_0001910_1_TGCAGGTCATCACCGAAGAACGAAT | 575 | AAAACAATTAAAAAAAGAAGATTTGTGTATGCAAGATGTGTG | C | C | M | A | GYSNP0557 |
| ACATATCTTCCTCACATCCTTCTTCTTTCCTGATATAATCA | | TTTATGTGTGTGTTGATGAATGCAGCTCGGCAGATGGG[C/A]T | | | | | |
| GAAGCTAATTTGCACAAATACGGGC_NODE_1_length_659_cov_ | | TTCATGATACTCGAGAATGCCCGTATTTGTGCAAATTAGCTTC | | | | | |
| 134.581177 | | TGATTATATCAGGAAAGAAGAAGGATGTGAGGAAGA | | | | | |
| RAD_kmer_0001917_1_TGCAGGTGCAGAAAATTAGACATTA | 202 | TTTTAAGAGCCTTGCGTCTAGGCCCAAGGCTTGCTCTTTGCGC | — | G | G | K | GYSNP0558 |
| AAAGAAATTGGATGTTTCTTATCTTGATGCATGCTCATGA | | CTTTGACCATCTATATAGTATATACATTATCTACTGG[G/T]AG | | | | | |
| TATAGTCATATATTACAGCTAACAT_NODE_1_length_610_ | | GAGTGACTTATGTATTAGTTTTAAGATGATACCCACACAACA | | | | | |
| cov_87.639343 | | GGTGTAGATTCTAGGTATGATATGAAGACT | | | | | |
| RAD_kmer_0001926_1_TGCAGGTTCTGGCTCATCATCCATC | 271 | ATTCGGAGCCGCTCAACGTATAAATCATGCGGAAATTCTTCA | W | W | A | W | GYSNP0559 |
| AGGACCATCTTGTTTTGTTTTGATGCTTCATGCTATGCACA | | TTTGTAATTTCATCATCTTTCTTTGGGTCGTTCTTAAT[A/T]CG | | | | | |
| ATCACCGTGATACAAATAAATATCT_NODE_1_length_599_cov_ | | CACACTGGATTTTCTAGAAAAAACCTTTCCTAGCCTGCAAGC | | | | | |
| 151.978302 | | ATAAAGACTTAGCATCAAAAAATTCTCTACATAAAA | | | | | |
| RAD_kmer_0001932_1_TGCAGGCTTCCACCTTTGGGCGCT | 85 | TTTTGGTAAATCATCAGACGAGTATGAACTTTGACCTTGATG | G | G | A | G | GYSNP0560 |
| CATGGAGTAAGTGCACAAGCCTCTTTGATTTGAAAT | | ACTTTGACCACTATAACCAACAGGTAAAATATTTACAG[A/G]C | | | | | |
| CATGTTTTAAAATGTATTTTGCATTT_NODE_1_length_608_cov_ | | AAATGATTGACCTGCTAACAACACACAGATGTCATCAGATCC | | | | | |
| 134.911179 | | TAATTAGATAAAAATGTCAAATTACTTGAATTTTTAA | | | | | |
| RAD_kmer_0001937_1_TGCAGGTACAACCAGATCCAAAATT | 340 | TTAGCCATTTGCTGGATAAGTTCCTGCACAAATGATAAACCA | K | K | T | T | GYSNP0561 |
| ACGCTTTCATTCTTCGTTCTCTTTTGTTCCTTTCTTATCATC | | CTTCATTAACATTTGGTCAGATTTGTAACATATGCTCG[T/G]C | | | | | |
| GTGTTGAGAATCATGTTGACGAGC_NODE_1_length_399_cov_ | | AACATGATTCTCAACACGATGATAAGAAAGGAACAAAAGAG | | | | | |
| 215.877197 | | AACGAAGAATGAAAGCGTAATTTTGGATCTGGTTGTAC | | | | | |
| RAD_kmer_0001949_1_TGCAGGCTCCGCTGGGACTTCTTC | 403 | CTTGGTAAGTTACCAAGTTCCAAACATGTAAGTTACCAAGTT | Y | T | C | — | GYSNP0562 |
| TCTGCAGCCCCACCCTCCACCAAATTGTGAGTTATTATTTT | | CCAAAAATACTGTACATTCAACAATATTCAAGTTAATT[C/T]C | | | | | |
| TTTTTTAATTTAATTGAATTGCTCA_NODE_1_length_530_cov_ | | CAAAAACACATGAGCAATTCAATTAACAAAAGCCTCCACAGT | | | | | |
| 88.739624 | | CCACAAATTATAAAACACATGAGCAATTCAATTAAAT | | | | | |
| RAD_kmer_0001950_1_TGCAGGCATCCACTGAAAGGCTGCA | 134 | GATGGCGGGAATTGCTGATTATGCTAGATTTATATTGTAAAC | — | G | R | G | GYSNP0563 |
| GGCATTAGCAGCCTTCAACATGAAGCCAGATGGTCTTAAG | | CAGTACAGCAGCCGCACTCACTACGAGAAGACGGCTTC[G/A] | | | | | |
| CATCTATAACGTTTAGAAATTTTAAT_NODE_1_length_380_ | | TCGTACGACTCAGAGTTCAGCTTCTTTTGGGACGCCATTACCA | | | | | |
| cov_76.800003 | | CACTTCAGAACGGTAGCGTTTTTGTTTCTTCTAATAT | | | | | |
| RAD_kmer_0001950_1_TGCAGGCATCCACTGAAAGGCTGCA | 188 | CGCACTCACTACGAGAAGACGGCTTCGTCGTACGACTCAGAG | — | C | Y | T | GYSNP0564 |
| GGCATTAGCAGCCTTCAACATGAAGCCAGATGGTCTTAAG | | TTCAGCTTCTTTTGGGACGCCATTACCACACTTCAGAA[C/T]G | | | | | |
| CATCTATAACGTTTAGAAATTTTAAT_NODE_1_length_380_ | | GTAGCGTTTTTGTTTCTTCTAATATTTTCTATACTATGAAATTA | | | | | |
| cov_76.800003 | | TGTTCTGATTTGTTTTTTATGCTCTACTTTTCTT | | | | | |
| RAD_kmer_0001957_1_TGCAGGCGGTGCGGCGGGACCG | 208 | CGCCACACACAAATAAATAAACCCTAGCCGTCACCACTTGTG | T | K | G | T | GYSNP0565 |
| TAGTCCAATAGGCGGTGGAGGCGGCGTGGCGGCAAGGAG | | TAAAACCCTAGACAGGGCGGTGTGCCGCCATTGCCGCC[G/T]C | | | | | |
| GCGGCATTACGGAGAAGCGGAATCAATC_NODE_1_length_ | | CGCCCTGTCCAGCCGCTGTTCGTTTTAGTTTCCCGTTCACTTTT | | | | | |
| 643_cov_68.688957 | | CTTTTTCTTATGTCCTGTTCTAAATAAAGTCTCAA | | | | | |
| RAD_kmer_0001957_1_TGCAGGCGGTGCGGCAAGGGACCG | 266 | GCGGTGTGCCGCCATTGCCGCCGCCGCCCTGTCCAGCCGCTG | — | Y | T | C | GYSNP0566 |
| TAGTCCAATAGGCGGTGGAGGCGGCGTGGCGGCAAGGAG | | TTCGTTTTAGTTTCCCGTTCACTTTTCTTTTTCTTATG[T/C]CCT | | | | | |
| GCGGCATTACGGAGAAGCGGAATCAATC_NODE_1_length_ | | GTTCTAAATAAAGTCTCAATTTTCGGTTCTAGATTATATTCCA | | | | | |
| 643_cov_68.688957 | | GCCACGATTCCTAGATCTTTCTAATAAACCATCA | | | | | |
| RAD_kmer_0001957_1_TGCAGGCGGTGCGGCAAGGGACCG | 365 | TCAATTTTCGGTTCTAGATTATATTCCAGCCACGATTCCTAGA | G | K | G | T | GYSNP0567 |
| TAGTCCAATAGGCGGTGGAGGCGGCGTGGCGGCAAGGAG | | TCTTTCTAATAAACCATCATCCATACAATCCTTGTAG[G/T]AC | | | | | |
| GCGGCATTACGGAGAAGCGGAATCAATC_NODE_1_length_ | | GTTTATGGCCCACACCATTAATTTGGGTTTGACTCAACAAAA | | | | | |
| 643_cov_68.688957 | | GTTAAAGTGGATAACAACTGACGGTATCGAAAAATA | | | | | |
| RAD_kmer_0001957_1_TGCAGGCGGTGCGGCAAGGGACCG | 482 | CAACAAAAGTTAAAGTGGATAACAACTGACGGTATCGAAAA | G | K | K | G | GYSNP0568 |
| TAGTCCAATAGGCGGTGGAGGCGGCGTGGCGGCAAGGAG | | ATAGAAGAAACTTCAAGCACGCGGCCAAGCAAGCAGGAG[T/ | | | | | |
| GCGGCATTACGGAGAAGCGGAATCAATC_NODE_1_length_ | | G]AATGTGTTTCCTCCGTCGCAGCCGCTGATCATGATCAAGCC | | | | | |
| 643_cov_68.688957 | | CTTGCCGCTAGTCGTCTTGCTGCTGCTGTTCATCGCAAC | | | | | |
| RAD_kmer_0001959_1_TGCAGGGATCACATCATAAGAAAA | 247 | TTCCATCCGGATGAGGGCCTAATTTGGAGGATGTTTGTTCAAT | W | W | W | T | GYSNP0569 |
| AAATAAAAAATAAAACCCAGTTTGCAACAAAGAAAATCA | | TGCTCTTTGGCTATTCAGATTTCAGATTACGTGTACA[A/T]TAT | | | | | |
| AAACAACTTCGCAGAATACATCACGTGA_NODE_1_length_ | | CAGCATTTTTTCTGGTTATGTTGGATGTCACGCCTTAATGAAT | | | | | |
| 528_cov_106.490532 | | TATGCATTTGTTCTTCTCACAGGGGTTAACACCA | | | | | |
| RAD_kmer_0001961_1_TGCAGGGTTTTGGTCAGCTTGTTTTT | 380 | TTTTATGTTATTGATCTTAATGTAAAGTTGTTAATTATTCGCCT | T | G | T | G | GYSNP0570 |
| GTCAAGTTCCTGCGAGTTCAGGCCAGTTTTGGTGTTGACT | | TCCAATAAACAAATTAGTGAGGTTTCCACTATTAGG[T/G]TTT | | | | | |
| GGACCAAGGCACTAGCCTTGCATGT_NODE_1_length_604_cov_ | | GTACAAGGATTAAAAAAACATTTGCAATGAAAGCGTATACA | | | | | |
| 82.097679 | | ACAAATATCTTAAAAATTTCTGCCAGTGATCTACAT | | | | | |
| RAD_kmer_0001963_1_TGCAGGGATGATAAAAACTACAATC | 478 | TGTAATGTGAGTTGTGAGTCTGTAACAAAATGACAATATATT | R | R | R | R | GYSNP0571 |
| AGATTCAAGACCACATGCATGTTGAAGAGGTTCAATCGG | | TATATTGTTGTATGTGCAACATAAGTCAGTGAGTGTTC[G/A]T | | | | | |
| ATATACAACCTGAGGATTTGTACAA_NODE_1_length_642_ | | AATGAATTGTCCAAATGTGTGTTCGATTTGTGGCAGCTCTAGT | | | | | |
| cov_80.398750 | | ATGCAAAACAATATGTCATATTACTTACCAATGGGT | | | | | |
| RAD_kmer_0001974_1_TGCAGGACTATGAAAATAGCCAAC | 199 | TTATCTGAAAGTTAAAAGCTAATATTTTCTTTTGTTTTTTTAA | C | T | T | T | GYSNP0572 |
| GCCTCCTTACTGGTTAAATTTCAAGGGGTGGGTATTTTAG | | AGGAAAATAAAGATTCAATTAGGAATTCGCGTTGTAA[T/C]GT | | | | | |
| TCAATCTGATCAGAAAAAACACACGAG_NODE_1_length_615_ | | AACGCAACGATACCGTCTATTATAAATTTCTGCTCATAGTTTC | | | | | |
| cov_84.369102 | | TGATCGATTTAAAAACATGTTGATAACCTCCGATG | | | | | |
| RAD_kmer_0001977_1_TGCAGGATAGTGGATCCTAATAAGT | 300 | AAACACATGAGTTGATAAGAATGTGTGAAAGCTCACAGATG | A | — | R | R | GYSNP0573 |
| ATAGAAGATCTTGAATGAAAAAGATGGGACCCGGTCTTG | | GTGACCTCTAATCACTTGGTTCCTTTAAGGTTGTTTCAT[G/A]G | | | | | |
| CCAAGAGAAGCGAAAAAACTAAGGGTT_NODE_1_length_633_ | | ATGGTTTTATTGTTGTCTTTAGTAGAATCCAACAAGAAGGTG | | | | | |
| cov_59.191154 | | AAAGTAGAATGGTTCTCTAATTTTAAGGTACAATAGG | | | | | |
| RAD_kmer_0001983_1_TGCAGGAAAATCCAGATTTATGGAA | 95 | GTTTATGACTTTTGAGAAATCCAACCAAGGAATAATGTTTTTT | A | T | T | — | GYSNP0574 |
| ATGGCTAACTGGGCAGGAACAACCACCTGAACCAATTAA | | TAGGTTATAATAAGAACAGACAAAAGATATAAAAAAA[T/A]A | | | | | |
| CACAAATCCCGTGAGTTACACATCAAT_NODE_1_length_547_ | | AATAAATAAAAAACCAAACCGATCATACTGATGTTTAAATAC | | | | | |
| cov_117.511887 | | AACAATCTGGTCTACTAGAGAGAAAAATACATTATTT | | | | | |
| RAD_kmer_0001983_1_TGCAGGAAAATCCAGATTTATGGAA | 221 | CAATCTGGTCTACTAGAGAGAAAAATACATTATTTAGACCTC | T | T | A | T | GYSNP0575 |
| ATGGCTAACTGGGCAGGAACAACCACCTGAACCAATTAA | | AAAACCATGAGGGCTTTTATACTATAGCAGCCTTATTC[A/T]T | | | | | |
| CACAAATCCCGTGAGTTACACATCAAT_NODE_1_length_547_ | | GGTGCTTATAACATATGAAGTAAACTTATTCGTAAAAAGAGT | | | | | |
| cov_117.511887 | | TGTATATATATAATTACTGATTACCGACAATCGGGCT | | | | | |
| RAD_kmer_0001986_1_TGCAGGAAAAGTAAAACAAATAA | 115 | CAAAGACGCGTTTAACTGCACACGAAGTTTTATGTAAGTTCT | T | T | G | T | GYSNP0576 |
| AAACTTTTTTCTAACGAGCTGCTTCTTCTTTTACTAGATTT | | ACAAATCATTACTTGTATTTGGTAAATCTGAGCTAAAA[T/G]T | | | | | |
| TTAACCATTTGACCCAATATCAATAA_NODE_1_length_589_ | | TCGTTATTTTTACATAATCAATTATGAACTTGTACATTTTTAA | | | | | |
| cov_90.242783 | | AATAATTATGCATTGATTGATATGCCTGTAATAATG | | | | | |
| RAD_kmer_0001988_1_TGCAGGGGCTGGTGGAATACTATTA | 184 | TTTAATAAATGCATTGAGCATGTAAAAAGTTGTTTAAGAGA | S | C | S | C | GYSNP0577 |
| AGTTCAAACTCATCAAGGAAAATGTTATCTTTCACTTTAA | | GGGAATATGCAAAAAAATAATATTGATGATATAGTTCT[G/C] | | | | | |
| CACACTCACCCTGATACACATCGACT_NODE_1_length_560_ | | GTTGGTGGGTCGACTAGGATTCCCAAGGTACAACAAATGCTG | | | | | |
| cov_96.848213 | | AAAGAATTTTTTGACAGGAAACCACTTTGCAAGAGCAT | | | | | |
| RAD_kmer_0001988_1_TGCAGGGGCTGGTGGAATACTATTA | 242 | ATAATATTGATGATATAGTTCTGGTTGGTGGGTCGACTAGGA | R | G | A | G | GYSNP0578 |
| AGTTCAAACTCATCAAGGAAAATGTTATCTTTCACTTTAA | | TTCCCAAGGTACAACAAATGCTGAAAGAATTTTTTGAC[A/G]G | | | | | |
| CACACTCACCCTGATACACATCGACT_NODE_1_length_560_ | | GAAACCACTTTGCAAGAGCATCAAAGCGGATGAAGCTGTCGC | | | | | |
| cov_96.848213 | | TTATGGTGCAGCAGTGTTGTCTGCAAACTTGAGTGGT | | | | | |
| RAD_kmer_0001988_1_TGCAGGGGCTGGTGGAATACTATTA | 475 | GTCCATAGATATATGAGTGCCATAATCCCAAGAAACACGCCA | M | A | M | A | GYSNP0579 |
| AGTTCAAACTCATCAAGGAAAATGTTATCTTTCACTTTAA | | ATACCCACCATGAACGAGATAATTTATGGGACAGATGT[C/A] | | | | | |
| CACACTCACCCTGATACACATCGACT_NODE_1_length_560_ | | GATAACCAAGAATCCATTATAGTCGATGTGTATCAGGGTGAG | | | | | |
| cov_96.848213 | | TGTGTTAAAGTGAAAGATAACATTTTCCTTGATGAGTT | | | | | |
| RAD_kmer_0001990_1_TGCAGGGGTATTTACTCATCAAGAA | 360 | TGCCTATACCAAGAACACGCCTCATTTTATCAACCGCGCCAA | Y | Y | Y | Y | GYSNP0580 |
| GTGTTTTCATCTATTTACAACACCATCAAGCAAGTCTTCA | | AAAAAGATTATAAAACAAATGTATGGAATGTTAGAAA[T/C] | | | | | |
| AATGTAAGTGCTGCAACTTTCTTTAA_NODE_2_length_476_ | | ATAAAACTAACCATAACCCATCCCCATTCGTCAGCAAACGAA | | | | | |
| cov_49.321430 | | GGCACATGGGTCGTGTATGCAACCACATCTGCAATAAA | | | | | |
| RAD_kmer_0002000_1_TGCAGGTAGCAAATAAACCCTAGA | 375 | TTTTGCACTTTGCTAACTCAGGGCAGGTTTGCTGCGCTACGCC | G | K | G | K | GYSNP0581 |
| ATTCAAAATAACAAACACTTAATCAGCCTATAGATTTTGA | | TGGCCTGATCAGGGCGCCTTTGATTACATAGGACTTG[G/T]TA | | | | | |
| CCTAAAAAGATAGAAGGCCAGAGCTTC_NODE_1_length_554_ | | TATTTGATTTATTTGAAGTGGATTTATGCAACCTAGATGTCAT | | | | | |
| cov_83.525269 | | CGAAATGAATGCAATTGGTGAGTGGTGACGCTGGA | | | | | |
| RAD_kmer_0002000_1_TGCAGGTAGCAAATAAACCCTAGA | 493 | GATGTCATCGAAATGAATGCAATTGGTGAGTGGTGACGCTGG | A | M | M | C | GYSNP0582 |
| ATTCAAAATAACAAACACTTAATCAGCCTATAGATTTTGA | | AAGAATTATGCATCTGGATGAAAGATACTTTAGAAGCT[C/A]T | | | | | |
| CCTAAAAAGATAGAAGGCCAGAGCTTC_NODE_1_length_554_ | | GGCCTTCTATCTTTTTAGGTCAAAATCTATAGGCTGATTAAGT | | | | | |
| cov_83.525269 | | GTTTGTTATTTTGAATTCTAGGGTTTATTTGCTACC | | | | | |
| RAD_kmer_0002006_1_TGCAGGAAGTTGGTGATTCACCCAA | 118 | GAATTATCTATTAGTATTATATTTGAATTATCTATATTAGTGT | T | C | C | C | GYSNP0583 |
| ATGGCAACAAGAGTAAAAAAGCCGGAGAATTCTTGTCTG | | TAGGTTTAAATTATGTTTCCTATTGTTTAGATAATTA[C/T]CAT | | | | | |
| TATATCTAGCCATGGCGGACACGAGCG_NODE_1_length_457_ | | TTAGTACTTTTGGAGTTCTAGTAGAATTAGGATAGTGGGCCT | | | | | |
| cov_100.647705 | | ATAAAAGTCTTTGTTTCCTTTGTATTTTTCATTCA | | | | | |
| RAD_kmer_0002010_1_TGCAGGTTATAGATGGGTTCCATAC | 110 | AGTGTGTGATATAACTTGTTGAAAACAAAGTAGTTAAGTAAT | A | M | M | — | GYSNP0584 |
| ACTATGATTTGAAACTGTTTTCAACCTGTTATACCTGATTC | | TATACATTTCATCGTTAGAAGACAAAATCAAATCTCCT[C/A]T | | | | | |
| AAACAGGTCAAAACCCGAAAAAACT_NODE_1_length_637_ | | CACTTTTAGTTATGATATTCCAATCCATACTCTTACATGATCT | | | | | |
| cov_93.921509 | | TTTTATAAATCACAGTATAAAGACATCATGACCATA | | | | | |
| RAD_kmer_0002010_1_TGCAGGTTATAGATGGGTTCCATAC | 241 | AAATCACAGTATAAAGACATCATGACCATATCTTGACTGACC | M | C | C | — | GYSNP0585 |
| ACTATGATTTGAAACTGTTTTCCCTGTTATACCTGATTC | | ACTTCGAACTTCTAAAACAACCTGATGCATTGTGCTCT[C/A]T | | | | | |
| AAACAGGTCAAAACCCGAAAAAACT_NODE_1_length_637_ | | ACAGTAAACTCATGAACAAGACCATTGATCTCAATCCAACTA | | | | | |
| cov_93.921509 | | CACCCGGATTGTGTCTTAAGTTTCAATTTCTCATCA | | | | | |
| RAD_kmer_0002010_1_TGCAGGTTATAGATGGGTTCCATAC | 292 | TTCTAAAACAACCTGATGCATTGTGCTCTCTACAGTAAACTCA | R | G | R | — | GYSNP0586 |
| ACTATGATTTGAAACTGTTTTCAACCTGTTATACCTGATTC | | TGAACAAGACCATTGATCTCAATCCAACTACACCCGG[A/G]TT | | | | | |
| AAACAGGTCAACCCGAAAAAACT_NODE_1_length_637_ | | GTGTCTTTAAGTTTCAATTTCTCATCAGCTTCCTCGTCGTTAAT | | | | | |
| cov_93.921509 | | GAATCTTCCCACTTGTTAATCATCAGGTAAATGT | | | | | |
| RAD_kmer_0002010_1_TGCAGGTTATAGATGGGTTCCATAC | 512 | GCTGCTTCAACTAATTGCAGGTCATGTTTTATTAAACAAGCAT | G | A | R | A | GYSNP0587 |
| ACTATGATTTGAAACTGTTTTCAACCTGTTATACCTGATTC | | TCAGAACAGACCTCGAATAACCACATCTGGTTCCATA[G/A]GC | | | | | |
| AAACAGGTCAAAACCCGAAAAAACT_NODE_1_length_637_ | | ATGTTCTGGATCAACTCCATTGCTTTAATAATATGCCCAACCC | | | | | |
| cov_93.921509 | | AACAAAAAAAGATCAAGTTTTTTCGGGTTTTGACC | | | | | |
| RAD_kmer_0002014_1_TGCAGGCATGTGGAGCCAAAAATGT | 133 | GTTAAAATTAGGGTATTGCTCTTTGCATGATGAGTCTAATTGA | — | A | W | A | GYSNP0588 |
| AGACCTGCAAGCTTTGAGCTCACTAATTAAATAGAACACT | | ATTGGCATAATGATCATGTTTTCTGATTTTTTTTGTT[T/A]TAT | | | | | |
| GATCATCTAGGAAGTAGAATTAAATT_NODE_1_length_581_ | | GTGAACGCCCAATTGCAGTGAGTGTGAGTGTATTCTATCAAT | | | | | |
| cov_80.227196 | | ATGCGTCGGATTGTAGTAAATGAGCGGGTTTACTA | | | | | |
| RAD_kmer_0002018_1_TGCAGGCTATGAGTACAGGGGTCAA | 479 | GGTGATCAAAACCTAGAAAAGAAGCAGAAGTTTTGTATAATT | C | T | T | T | GYSNP0589 |
| ACGGGTCAGGTTTGATAACGGGTCAGAACAGACCGCGTT | | AGGTGCAATTTCAATCCTTTTATATGAAAACGAATCGG[T/C]T | | | | | |
| GACCTGTCACTATTTTTTTTATATAAT_NODE_1_length_631_ | | CAGGCTAGATTTGATTGATAGTGGGTCAAAAGGGTGAAAATA | | | | | |
| cov_152.486526 | | TTATATATTAGTGAAGTTAAATATTTGACAGTATAGT | | | | | |
| RAD_kmer_0002022_1_TGCAGGACTACAGTCCGCCGCCACG | 432 | CCCCGCAGTTGGAGCTGAAGGAAAATAGAAGCTCAAACTGA | G | — | A | G | GYSNP0590 |
| CTACTAGTTAGTTAATTTCTACCCCGCTATCACACCGCCAC | | AATAAAACATAAAAAATAATAATAACAAGATAAACGTGCTTC[A/ | | | | | |
| TTTGCACGCCCCTTACAGGATTACA_NODE_1_length_577_cov_ | | G]GTCGAATCAGTATTCCGTACTTTGCCTACAAATCCATATTT | | | | | |
| 53.542461 | | TGTTTAATTTTTCCATCCATAGCCTTCATAGACAGATCC | | | | | |
| RAD_kmer_0002029_1_TGCAGGACTTCCCGCCCAGGACAAA | 211 | CATATATAATAATAATTATGTAAAGATATATTTGATAAAACA | T | T | C | T | GYSNP0591 |
| CCAGTGCCCCGAAACCCAAACTGGGTACATTCATTTGTTT | | TATATAAATAATAAGCTCGTTTGGGCTTGTTTATTAAA[C/T]G | | | | | |
| CTTTTGTTTCAGTACCTACAAAGGGA_NODE_1_length_574_ | | AGCTCCGATAGGATTAAGCTGATTTAAGTACGGTTCAATTCA | | | | | |
| cov_110.118469 | | AACTTTTAGCGAGCTGATCTTGAGTAGCCCTGTGCTG | | | | | |
| RAD_kmer_0002044_1_TGCAGGAACTAAGGAGGCTGGTGTT | 107 | CTCCAATTTCCATGTAACGAATGATACAGTGAACTACATGGT | C | — | G | C | GYSNP0592 |
| TATGCTTATACATCGGTAACAAATCTGACCATTATCTATC | | TAAAGGTGTATAATACTATGTACGATGAATGGTCCAAA[G/C] | | | | | |
| ATGATCAATCTTTTTTGGATAATATG_NODE_1_length_588_ | | ATCCATCTAAGACACATCCATGTGGGACAAAATCATTGAGAA | | | | | |
| cov_108.025513 | | TGGTACCTCTAGTTTCGTGTTTGTCAAGTCCTAGAGAC | | | | | |
| RAD_kmer_0002047_1_TGCAGGTGCAGATGAACCAAAAGG | 119 | CTCTCAATCCTCTTACTCATACTTCTTGTATGGTTCATACTCCA | Y | C | Y | C | GYSNP0593 |
| CAAAACAACCACACAATTCACAATAAACATATACCTGCC | | CCCCTCCAAACCAGAGTTTGCCCTCAAAGAAGCCGA[C/T]ATT | | | | | |
| AGAAACCCAAGTTCCTACCTTCCACCGG_NODE_1_length_495_ | | TACCAACTCGGCCTCTCAGGATCTCACCTTCTCAACTCATCCA | | | | | |
| cov_138.898987 | | TTCAAATCACTCTTCTTTCAAGAAACCCGAATCA | | | | | |
| RAD_kmer_0002047_1_TGCAGGTGCAGATGAACCAAAAGG | 374 | GAAAACAAGGATACCAATCTTTTATCGATGGTTTTAGTTGCA | K | G | K | G | GYSNP0594 |
| CAAAACAACCACACAATTCACAATAAACATATACCTGCC | | AACGGGTTACCTGTGGACCCATCTTTCGGTTATGAAAT[G/T]G | | | | | |
| AGAAACCCAAGTTCCTACCTTCCACCGG_NODE_1_length_495_ | | GTCGTGATCAAATGGTGGGAAGGTTGGTTTTAAATCTCAAGT | | | | | |
| cov_138.898987 | | CAAATGGTCAACTCCGGTGGAAGGTAGGAACTTGGGT | | | | | |
| RAD_kmer_0002056_1_TGCAGGTACTAATAATAAATCTTGG | 491 | ACAAATAGAGAGAATGAGACATCAAAAGGTTCATTCCTGTAA | M | C | A | M | GYSNP0595 |
| TTTTTATTTATTTTTTATTTTTTTTATTTTTTGGTTAGGGTTT | | ATACCCTTCTTTCCACCACCCATTCTTTGTCTTGCTTA[C/A]AC | | | | | |
| TGTTGATTTGTTTGTAGGCTTTG_NODE_1_length_609_cov_67.059113 | | GCGTCTCTTACTCTTCTTCATCAGTCAGCATCGTCTCTTACAA | | | | | |
| | | GATTAACACAAAGCCTACAAACAAATCAACAAAAC | | | | | |
| RAD_kmer_0002061_1_TGCAGGACACACCACCGGGGCAGG | 208 | GGTGGTGATAATGTGATAATGTTTTTAGATGTTTAAAGAATG | R | A | G | A | GYSNP0596 |
| GTGAAGGACAGCCGCACCTCTCATCACATATAACACCGCT | | ATGGTATTATGATGTACCAACTGACTAAGCATGTTTTT[G/A]G | | | | | |
| TCCTCTCATGTCCGCCATCCTTAATGT_NODE_1_length_553_ | | TGTTTTTAAAGCTCCTTATTATGATGCTCTTATGTATTCTTTGT | | | | | |
| cov_114.037971 | | TAAATCTCATAAAAAACTATTACTTTAATAGTAAA | | | | | |
| RAD_kmer_0002062_1_TGCAGGTCAACAGGCTGCTTCATTT | 411 | ATACTTTTTTTATTTATTTTTTAATTCCGTCTCGCGCTTACGCC | S | S | S | S | GYSNP0597 |
| GTACTTCTTTTGTTTTCCTGCATATACACATTTATTATAAT | | TCGGTTCGCCTCGAGGCTTACGCCTCATGAGGTAAA[G/C]GGA | | | | | |
| CAAGGTTTAAAAGTACAAAAACGAG_NODE_1_length_492_ | | AAACGCCTCGAGGCTCGTTTTTGTACTTTTAAACCTTGATTAT | | | | | |
| cov_90.745934 | | AATAAATGTGTATATGCAGGAAAACAAAAGAAGT | | | | | |
| RAD_kmer_0002063_1_TGCAGGAAGTGCAAGATCATGACTT | 294 | GGAAGGACCTCGTCTTTAGGTTTTAGATCCCCAAAAGATATA | T | T | Y | Y | GYSNP0598 |
| CATGAACCAGTTTCAGATGATGAAACCTTATAACATGGTT | | GTAAAAGTATTGGGAATATCAGGAACCGGCTTTGGTTT[T/C]G | | | | | |
| GGCGACTTCAGTGTCGATTCCTTTTC_NODE_1_length_521_ | | GATTAGGGTTTTCATTCGCTGGAATACTTTGTTTGTTGATCAT | | | | | |
| cov_163.190018 | | ATTGTTGTTCAGTTTATTAGTAGACAGCTTTTTCAT | | | | | |
| RAD_kmer_0002064_1_TGCAGGCGATTATAGATCCTGGAAG | 468 | CAAAGGCTTGTTCCTAATGACCATGGTGAGCAAAACCAGATG | Y | C | T | Y | GYSNP0599 |
| CATTCATCGTTGTATACTAAGAAAGAAGATTCACATGAAC | | TCATGACATTGACTATCCTTAACCTCAACTCTTCTATG[T/C]AT | | | | | |
| GAGACAATAATAAATTTGTTAATCGA_NODE_1_length_574_ | | CCTTTGGGCATATAATATGTTCCACATTTCGAATACATGTCGA | | | | | |
| cov_126.653313 | | TTAACAAATTTATTATTGTCTCGTTCATGTGAATC | | | | | |
| RAD_kmer_0002074_1_TGCAGGACTAGCATCCCTTGGTCGA | 182 | GTTTTTGTGTCTTCTTTTAGTTAAAAGTTAAGTTTATAATCTTG | A | R | G | R | GYSNP0600 |
| TTTTGAGGGCTAGAGTGGTGTTGACCGCTCGTCACCACCG | | AAACACACCATGTATGTTAATTGCATAAAATATATA[G/A]AA | | | | | |
| ACTCACAACTCGTGTCTGTAGTGACG_NODE_1_length_623_ | | GTCTAGACCAGAAAGAATGGATGGTAATCTTTTTCGAGTTTT | | | | | |
| cov_109.593903 | | GATGACTTTTTAAATTAAAAAGTTTATTGTTTGTAT | | | | | |
| RAD_Kmer_0002075_1_TGCAGGTTTTATTGGGATTCTCTGTT | 127 | ATAAACATCATCTTTTTCAATCTTCAGATGAAAAAATGGATTC | Y | Y | C | Y | GYSNP0601 |
| CTTGTTGGTGCTAGTGTTTGGGTGGATCTACTTGTAAGTCC | | AATCACCTTTCTTAGAACACAAAATACCCTAGCAACT[C/T]TA | | | | | |
| CTTATATTTGTTGTAAATGGGTTA_NODE_1_length_633_cov_ | | TATTAATGTTGGGAAATTTATGAGCAACAAGTTTAAAAATAA | | | | | |
| 119.352287 | | CTGTAATACCCAAATATTTGTTATACTAAATTTTCA | | | | | |
| RAD_kmer_0002075_1_TGCAGGTTTTATTGGGATTCTCTGTT | 382 | TCATCATCATCTGATTTCATCAACTGCTGGGGCCGCAAATCTT | M | M | A | M | GYSNP0602 |
| CTTGTTGGTGCTAGTGTTTGGGTGGATCTACTTGTAAGTCC | | ACTTCTGCCGGGCGGGCTACTACAACAGGTTCATCAG[A/C]AA | | | | | |
| CTTATATTTGTTGTAAATGGGTTA_NODE_1_length_633_cov_ | | AGAGGGCTTTGATAAATGGTGGGGTTTTTAGGGGTCGACGTC | | | | | |
| 119.352287 | | CAGTCATTTTCGGATAAACGTCTTCTAGGAAATAGT | | | | | |
| RAD_kmer_0002078_1_TGCAGGTAAAGTTTTTAGGGAATGG | 207 | CCATCGTGTTTTATCACCATCGTGTTAAAACATCACATCTGAA | R | A | R | R | GYSNP0603 |
| GATAAGAACCGAAGACCACCCGATTTTGTACAGTACGGTT | | CCCATTGTGTTTTAAAACTCATGTTAACTACTCATCC[G/A]AA | | | | | |
| CAGGTTTTGGTTTTTTTCGGCCTGGG_NODE_1_length_418_cov_ | | CTTATTGTGTTTTTATCAACCCATTGTGTTTTAAGCTCATGTTA | | | | | |
| 94.956940 | | ACCACACATCTGAACCGATTGTGTTTTAAACTCA | | | | | |
| RAD_kmer_0002081_1_TGCAGGGCCGGCCCAATGGCTATCA | 479 | CAAACTGTTACTAAAACCAAAAAACAGAAACCGAACGGTTT | R | G | G | A | GYSNP0604 |
| AGCTAAGCAATTTCTTTAGGTAGAGGTGAAAAAAACAAG | | TCAAAAACCGAAAACCAAACCGAACAGTGCACACCCCT[G/A] | | | | | |
| GTATTATTGCCTAAGTTAATTTATCCT_NODE_1_length_550_ | | CTATGTAGGATAAATTAACTTAGGCAATAATACCTTGTTTTTT | | | | | |
| cov_127.414543 | | TCACCTCTACCTAAAGAAATTGCTTAGCTTGATAGCC | | | | | |
| RAD_kmer_0002085_1_TGCAGGTGGTACAGAGTACAAGGA | 474 | CTTCCCCTTCCGCCATGAAAGTTCCTTGGGTAATAATTTCCTG | A | G | G | — | GYSNP0605 |
| ACAGTATGTACCTAGAAGATCCTACAACCAAAGAGGCAA | | GCTCTTCAAAGTATTGGTTACGTCCATTCTGGTAGGG[G/A]CC | | | | | |
| TGGCCGCAGCAGCGGTGGTGGTGGTCGT_NODE_1_length_ | | AGCTCTGCCGCCACTGCGACCACCACGCCCATTCCCGTAACC | | | | | |
| 595_cov_103.391594 | | TCTACCACCACCACGACCACCACCACCGCTGCTGCG | | | | | |
| RAD_kmer_0002090_1_TGCAGGCTTGTAAGTGCCTTTTAAC | 237 | AAGTAAAAGATAATAGTATATTTTTCTAAATAATTTATTTTTT | G | — | G | A | GYSNP0606 |
| ACTAGAAACCGAGAACGAAGTCGAGACGTCCAAATCCCA | | ATAGTATATCTACTAAGTAGAGTATTTAAAAGTAAAA[G/A]AT | | | | | |
| TTTCCATGCATCCAATTCTTGTGAGAG_NODE_1_length_585_ | | AATCAAATGGGTTTGGACCTTCCTAACTGCATTAGAGGAGT | | | | | |
| cov_91.158974 | | TGTGGGTAGGGGTGAAAAAAATTCTATTCTGGCTGG | | | | | |
| RAD_kmer_0002094_1_TGCAGGTACAAATTGACTTTAATTA | 258 | AAGCGTGCTAAGTTTTCTCCAGTTTCTAGCAAAACCCACCTGC | R | G | A | G | GYSNP0607 |
| TCCTAGTCGATACTCTCTCTGTTTTGGTCAGTGGCGGAGCT | | CCGAAGGCTCACTGGTGGTATAACCCGAACACCCTCT[A/G]G | | | | | |
| TAAATTTTTTTTGGGAGGCCGAAAG_NODE_1_length_603_cov_ | | ATCGAACTGAAGATCACAAAGATCTCAGTCTTCTTTCACTGA | | | | | |
| 43.467663 | | GCCACCATGCCCCCTCCAAAGTGTTGCCAGTGGGAAT | | | | | |
| RAD_kmer_0002094_1_TGCAGGTACAAATTGACTTTAATTA | 527 | GTCAGTAGTGTAAATTTATGCGTTTTCGTCTGAAAAATTTATA | R | A | G | A | GYSNP0608 |
| TCCTAGTCGATACTCTCTCTGTTTTGGTCAGTGGCGGAGCT | | CGTATTGGCTCCCCTCTCCCCGATTTTAAGGGCTTTA[G/A]GG | | | | | |
| TAAATTTTTTTTGGGAGGCCGAAAG_NODE_1_length_603_cov_ | | GTTTGGTAACTTTCGGCCTCCCAAAAAAAATTTAAGCTCCGC | | | | | |
| 43.467663 | | CACTGACCAAAACAGAGAGAGTATCGACTAGGATAA | | | | | |
| RAD_kmer_0002096_1_TGCAGGTTAAAAGTTCGTGTTAATC | 205 | TCGGGTTTGTCTACGCTTTAACGTAAATTTTGTTTGAAAACGA | G | — | T | T | GYSNP0609 |
| TAGGAAGACCGAATAGATCGATTTTGTTTACAATAAAAAT | | GTCGGTCAAATTATTAAATATCTTTTATGTTCGTTTT[T/G]AGT | | | | | |
| CGTTACCAACCCGACCATTTGTTTTT_NODE_2_length_515_cov_ | | TGTACAACGTTTTGACGTAAATTCTGTTTGGAAACAAATCGG | | | | | |
| 66.182526 | | GTCAAATGTAACATTTTGATTCGATGGTATAAGTT | | | | | |
| RAD_kmer_0002098_1_TGCAGGGCTGCGCATGGGCACCATA | 91 | TAATGATTTTGTTTGAACAGATGTTGGCAGATAAGGAACCTG | T | Y | Y | T | GYSNP0610 |
| GCAGCAACTGATCGAATCGACTGTCCCGCGTCTAGTGAT | | GTGAGAGCATCATTCGTCCTAGCAGCCGTGGACCCTCA[T/C]A | | | | | |
| CGTCAATGTGTCTTTGAAAATACGCC_NODE_1_length_589_ | | CTTGACTTTGACCCTAAAGGTCTATGATGGCGTTTATGCTCAC | | | | | |
| cov_157.558578 | | AAAGATATCACTGAAGGTGGGAAAGAGAATAAAGAC | | | | | |
| RAD_kmer_0002098_1_TGCAGGGCTGCGCATGGGCACCATA | 366 | ATGTTGGCCTATCGGAAGTTTAGAGATGGGGGAAAAGCTGAA | A | W | T | W | GYSNP0611 |
| GCAGCAACTGATCGAATCGACTGTCCCGCGTCTAGTGGAT | | GTTGATGAAATTTTAAGAAAAGAGAAAAATGAAAACCC[T/A] | | | | | |
| CGTCAATGTGTCTTTGAAAATACGCC_NODE_1_length_589_ | | TCAAGAATTGTTTACTCGTTTGGTATATCTCATGAACACCCGG | | | | | |
| cov_157.558578 | | GGACGTTTATTTTAACTTATATAAGGAGTTCAAATCC | | | | | |
| RAD_kmer_0002100_1_TGCAGGGGAATTGTGGTGAACAAA | 139 | CTCCCGGGGATGAGCCGGCCTGTGGTGACGTGGCGGGTGACG | M | M | A | A | GYSNP0612 |
| GCAATTTACATATTGTTGTAGCCCAATATAGAGTAAGAGA | | TAGAGGCCCATCCTCCATCCTCCATCCTCCACACCCCC[A/C]T | | | | | |
| ATAAAGATAGAAACATTGTGCAACATT_NODE_1_length_495_ | | AGCCTAATAAAACAACTCATTAACCAGACCCACTACTTTGAT | | | | | |
| cov_93.856567 | | TAATATCACACATAATAAAACATGTGGCATAAATAAA | | | | | |
| RAD_kmer_0002100_1_TGCAGGTTTACGACCGTTGTGTTTG | 131 | GTTAACGCCAGTAGAAAAATAAATGCCCGAATCCGATTCGCA | A | G | G | G | GYSNP0613 |
| GGTAGAACTCTTTGATGCGGGTCGCCTTTGCAGGGCCCAC | | TATTATTTATTATGGGAGACGAAAAGACAATTTTTAAG[G/A]G | | | | | |
| CCATGGGTCTAAATCTCCCCAGACTA_NODE_1_length_606_ | | ATAGACAAGTACAATCCGTAGATACTCGAATCTGATTTGTGT | | | | | |
| cov_79.212868 | | AGCATTCAATTCTCATTTTTTATTTTTTGCAAGAACA | | | | | |
| RAD_kmer_0002108_1_TGCAGGGTTAGATCCTGTACTTGCT | 483 | CTACTTCTCTTTCTTCAACCTAAGTAATAATTGAAAAGATCTC | C | C | M | M | GYSNP0614 |
| GAAACACTTCCTTCTGGTGTTGCCTATCACCATGCTGGTCT | | ACAAGATTCAAACTTTACAAAATAACATAAAATTTAC[C/A]AT | | | | | |
| CACAGTATGTGCACTACTATCACTC_NODE_1_length_592_cov_ | | CACTAACTGAAAAGGTAAGTGTTTGTGTGTGTTTGTTGTTTGG | | | | | |
| 183.442566 | | AGTGATAGTAGTGCACATACTGTGAGACCAGCATG | | | | | |
| RAD_kmer_0002111_1_TGCAGGATTGACTTTTCCAAACCTT | 150 | ATTATTATATTATATTATACCACAAGTTTTAGCTTAGAGTGAT | W | A | — | T | GYSNP0615 |
| CCTCCTGGTCAACAAAATTTAATTGGAAACATAAAAAGTT | | GAGCATTGAATCCACCATCTTTGTTTTTAGGTTGTGG[T/A]GA | | | | | |
| AAGCAAGGTTGACAAGATAATTATCA_NODE_1_length_484_ | | TAATGGGGACAATGGAGATGTTGCAGATGATCATTATCATAT | | | | | |
| cov_86.400826 | | GTTCTTAGTGAGTCTGTTAATTAAAAACTCCTGTTA | | | | | |
| RAD_kmer_0002115_1_TGCAGGTGATATGTGATCTGTTGTA | 330 | ACCGATTTTAAGTTGGTCTAACCTGTCTTGCCTGTTTTAAGTT | A | — | C | C | GYSNP0616 |
| ACAGTGTAATACTATCACCAAAGTTTAGGAAGCAGTAGG | | CGTCTTACCTGGCTCGACCCATTTCAAGTCATTCTAA[C/A]CC | | | | | |
| AATCCTTCACACCAAGATTCCATGCTT_NODE_1_length_590_ | | GGCCTGACTTGTTTGAACACATGGACCAAAGTTTTTATGGAA | | | | | |
| cov_98.064407 | | CAGTGAAACTAGGAATATCTACTTTGTGCAAAATAA | | | | | |
| RAD_kmer_0002115_1_TGCAGGTGATATGTGATCTGTTGTA | 473 | CTACTTTGTGCAAAATAATCTTATTAATTTTCTAGCAGGCTTT | A | — | A | C | GYSNP0617 |
| ACAGTGTAATACTATCACCAAAGTTTAGGAAGCAGTAGG | | TATCTTATTATATAAGTAATAATTTTATAAAAATCTA[A/C]TAT | | | | | |
| AATCCTTCACACCAAGATTCCATGCTT_NODE_1_length_590_ | | CTTAATGGAGCAGGTGACTTATGCCAAGAGCGAGTGAAGGTG | | | | | |
| cov.98.064407 | | CATTCTCAAGCATGGAATCTTGGTGTGAAGGATTC | | | | | |
| RAD_kmer_0002117_1_TGCAGGCGAACCCAAAGACGCCCC | 341 | AAATCGTGATTTGAAATTAGGAAGTTGAAGTGTAAACTAGGG | G | G | G | C | GYSNP0618 |
| CACCCTCCTAATATTTGAGGAAGCTATGATATATATAAGG | | GGTCGTAACAAAGAGTTGGAAGAGCAATGTTACATATT[G/C] | | | | | |
| CAACGGATTGACTATAGCAGTCTTGCA_NODE_1_length_548_ | | TCGAGTGATGGATGACGAATATATAAGAATTGCATGCCTAGT | | | | | |
| cov_87.182480 | | TTCCTAAACTAATTTTAAGTTTATGTTTGGAGTTAAAT | | | | | |
| RAD_kmer_0002121_1_TGCAGGATCGATTACACGAGGTTAT | 389 | ATAGTTGCTCTTGAACAACAGGTTTCACAAATATCATATGTG | A | A | R | R | GYSNP0619 |
| ATATAAACCAACAACACAAACAAGACACGTTATTAACTC | | CTATGCAAGTTTATTATCTATTATTCGTATTATTATTT[A/G]AA | | | | | |
| ATATGAGTAGAAAAAATTGGAACAAAG_NODE_1_length_593_ | | TTTTGACATATCTTTGTTTGTTAGTTATTAGCTGGTTAGTTTTG | | | | | |
| cov_125.021919 | | TCATTATCTTTTTCATTGTAATCTCTTTACACTG | | | | | |
| RAD_kmer_0002122_1_TGCAGGGTTTGGGACATGCGAAACC | 118 | TCAACTCCATAGCGTCGATAGGCTGCTAGCATTATCAGAACA | C | M | C | M | GYSNP0620 |
| TGTCAAAATCAGTTACAGCTCTTAAGGGCAACATAGGAGC | | AATGACATCTTATAAAATCGCCATGTGACCGCTAAGAA[C/A] | | | | | |
| CATTAGATCTATCCGCTACTCCTCAG_NODE_1_length_603_ | | ACCACCTTAAAAGAGTCAGGAGCAAATAAATGTATGGTACAA | | | | | |
| cov_111.474297 | | ATATATTGTAACATACAACATACATATCTAACCACCAA | | | | | |
| RAD_kmer_0002122_1_TGCAGGGTTTGGGACATGCGAAACC | 215 | CAGGACCAAATAAATGTATGGTACAAATATATTGTAACATAC | T | C | Y | Y | GYSNP0621 |
| TGTCAAAATCAGTTACAGCTCTTAAGGGCAACATAGGAGC | | AACATACATATCTAACCACCAAGAATTCGTAATAACCC[T/C]T | | | | | |
| CATTAGATCTATCCGCTACTCCTCAG_NODE_1_length_603_ | | AAAAATGAGTTCAATGGCCAATTTCTTTGCTAGCTTCTATACA | | | | | |
| cov_111.474297 | | TATCTCCATTATACACATGTTACACAATTTCTATGT | | | | | |
| RAD_kmer_0002122_1_TGCAGGGTTTGGGACATGCGAAACC | 348 | TTATACACATGTTACACAATTTCTATGTATAATTAAAAACCAT | T | C | Y | C | GYSNP0622 |
| TGTCAAAATCAGTTACAGCTCTTAAGGGCAACATAGGAGC | | CTCAAATTAGAGAATCAAAGTAAAAATGTTTGTGCCT[C/T]CG | | | | | |
| CATTAGATCTATCCGCTACTCCTCAG_NODE_1_length_603_ | | TCCCAACTCCAGTAGGCTGCCATATGTACGGTCCCAAACACC | | | | | |
| cov_111.474297 | | GATAAAAAGTGACTCTGTGTCAGGACTAAAAGATGT | | | | | |
| RAD_kmer_0002123_1_TGCAGGTGAAAGATTCAGTGAGATT | 256 | CAATATTCACTATCCGAAATTTTCGGATATCCGGTTTTCGGAT | T | — | T | C | GYSNP0623 |
| GTGGGTAGTCTATACTACATGGTTCTTGAGGTCCTAAAAA | | ATCCGAAAATCGGATAATCCGATCTTGAACACCCCTA[T/C]TA | | | | | |
| GAAATTATGGCCCAGAATTTGATGTA_NODE_1_length_443_ | | CCCTCCACTCCACCCATTATTCCACCCGTAACTTAGTGTAAAG | | | | | |
| cov_99.984200 | | AACTAAAAACCAACCTGCCCAAATCGGCGGGACAC | | | | | |
| RAD_kmer_0002127_1_TGCAGGCGCGCACGGATGACCTGCA | 460 | ATTGGTACTTATTGATCTTTTTCCTATGATTTTTCAACACTATT | G | — | S | G | GYSNP0624 |
| ATTCTTACCATTTGAGAAATAAGTACTAGTTTGTAATTATC | | GTTGATTATGCAACTGTAGGAGCTTCTTCTAAACTT[G/C]CAG | | | | | |
| TCATTTGAGTTGAAAATAATTAGAA_NODE_1_length_575_cov_ | | CCATCCTTGTAACATATCAATTCCAGGTACCAACACTTGGATT | | | | | |
| 137.490433 | | TAATTTCTAATTATTTTCAACTCAAATGAGATAA | | | | | |
| RAD_kmer_0002128_1_TGCAGGAAAGAAAATACAGGAAGA | 192 | TCAAGAATCATTTGGAGAAGTTATTGTTCCAAGATGTTGTTTC | T | T | G | G | GYSNP0625 |
| CGCTATAATCAACGATAACTCAGAATGATGACAAAATAT | | ACTTGTCACCATATTGTAAGTTTCTTTACTTTGACTT[G/T]TTT | | | | | |
| ATGATATTATTATATTTTTACTAACAAA_NODE_1_length_484_ | | AATTTCTCTTTCTTTTGTAATAAAAGTTGTTGACTATTTTCTAT | | | | | |
| cov_89.764465 | | ACATTCATCTTTTATATTCACTAGCTATACCCA | | | | | |
| RAD_kmer_0002134_1_TGCAGGAGATCTTCCCACTGTCGGA | 448 | TCATGATAGCACCATTAATGAAGATAATGAAAATGAGGAAC | A | G | R | G | GYSNP0626 |
| TCATAAACAAGCTGTTGCGGTTGGGTAATGTTGGCTTCAG | | AAGAGGCAATAGTTAAGAGCAACACTCAAGGTATCAATA[G/ | | | | | |
| CCTTTTGCAGCTCAACTGACTCTGTA_NODE_1_length_612_ | | A]CTCAATGATGTTCGACAGTTCTGTAACTGAAAGGAACCCA | | | | | |
| cov_206.130722 | | GGTGTGCATTTACAGTTCTACCATAATCTACCATATATTG | | | | | |
| RAD_kmer_0002135_1_TGCAGGTGTCATAAATAAGCGATGA | 398 | CTAATGAACGGTACGTCTAAGAGTTTGATTTAAAACCCTTCA | T | T | C | T | GYSNP0627 |
| TATCTTAAAAACGTATAGTACAGCTATAAAGGAGTTATTA | | AAACCGGATCGTGAGCCAGCGTCTCTAGCCATTACTCA[C/T]G | | | | | |
| TTTCTTTGTTTTTATTTTATTTTTTA_NODE_1_length_504_cov_ | | TACCATCTCAAAACTAAATAATATTATTAATTATTTTCATTAA | | | | | |
| 96.277779 | | AAAATAAAATAAAAACAAAGAAATAATAACTCCTTT | | | | | |
| RAD_kmer_0002142_1_TGCAGGCTCATGACGGTCGGGAAGC | 311 | AGCCAAACCAAACTATATCACAACACGGGTCGTGAAACCGA | W | — | A | T | GYSNP0628 |
| CACTCAACGGTGAGTGACGGCCACTCGAGTGCGTGTGTGA | | ATCAAAACCCCTTAAAACCAAACCATCAAAAACCCCTTA[A/T] | | | | | |
| TGACCAAATCGTATAAAAAAGGTGTG_NODE_1_length_510_ | | AACCAAACCTTTCACCGTTATTTCAGATCAAACTTCACGTTTT | | | | | |
| cov_108.619606 | | TCAACAACATTATGGTGAAAGATGCTGACGACATCAG | | | | | |
| RAD_kmer_0002143_1_TGCAGGTTACAAATGCTCTACATGG | 529 | ATTTTATATTTTTTTATATATGATATATAAACAAAAAATGGAC | G | T | T | T | GYSNP0629 |
| TCAACATGTTTCTTTTGACAAATGACTGAGACTAGGAAGG | | CCCTCTCTAGAAAACGGACCTTGTGCGGTAATCTGTC[T/G]CT | | | | | |
| AATCCTATACGAAGTTTTATGCTAAG_NODE_1_length_600_ | | AATACTTAGCATAAAACTTCGTATAGGATTCCTTCCTAGTCTC | | | | | |
| cov_66.955002 | | AGTCATTTGTCAAAAGAAACATGTTGACCATGTAG | | | | | |
| RAD_kmer_0002148_1_TGCAGGTTTTGTCATGAGGCCGCAA | 112 | ATTTAACAGTTACGAAATTAGCGGCTTAGATGACTCCCAAAT | — | K | G | K | GYSNP0630 |
| ACAGATTATAAGCTTATTTCACCGTAACGGTAAACTTGTA | | CCCAATGCATAGCACAAAATGGTCACTCTATTTTATCC[G/T]G | | | | | |
| ATTCTAAATCCTCTAAAAATAACGAT_NODE_1_length_565_ | | ACAATTAAATACATGATTTAACTTTTAGTATGCAAACCTCACT | | | | | |
| cov_82.899117 | | CCAAATCCTCAAAATATCCAATCAAAAAAAAAAAAA | | | | | |
| RAD_kmer_0002149_1_TGCAGGAATCAAGCCAACAAAATA | 498 | TGGATGATTCTTCCACCATAGCTTCATTTGACTGAAGGGATTC | G | A | R | A | GYSNP0631 |
| CCGAAAACAATTGGGCTTCTTTGAATGCTAGTACCATTAA | | AGTCTCCATTCTGTCTACAGATGGGCCAGTATGAAAC[G/A]GG | | | | | |
| TAGCTGTAATCGAAAATTCACTCCAGA_NODE_1_length_658_ | | CTGGGTTGAGCATTAGCAGATTCAACTGGATTTGTTGGAGGA | | | | | |
| cov_126.082069 | | ACCGTATGGGTTTCAGTTGAGACGAATGGGATTATG | | | | | |
| RAD_kmer_0002149_1_TGCAGGAATCAAGCCAACAAAATA | 558 | CAGATGGGCCAGTATGAAACGGGCTGGGTTGAGCATTAGCA | G | A | R | A | GYSNP0632 |
| CCGAAAACAATTGGGCTTCTTTGAATGCTAGTACCATTAA | | GATTCAACTGGATTTGTTGGAGGAACCGTATGGGTTTCA[G/A] | | | | | |
| TAGCTGTAATCGAAAATTCACTCCAGA_NODE_1_length_658_ | | TTGAGACGAATGGGATTATGTTCTGGCGTGCTACCTCTGGAG | | | | | |
| cov_126.082069 | | TGAATTTTCGATTACAGCTATTAATGGTACTAGCATTC | | | | | |
| RAD_kmer_0002151_1_TGCAGGTAACATTGCCTTTCACATT | 350 | TATCTATAATGACCATTGACCAGTGACTTATTTTGAGTCAATG | — | G | G | K | GYSNP0633 |
| GTCGCTAAAGACTGCATTTTCAACCCACTGTTATACAAAT | | TTATATCAACAGGGCTTTAGCCAATCAACCCAGTGGA[G/T]TT | | | | | |
| GGGTTAATTTGGGTTATGTTTTATAT_NODE_1_length_460_cov_ | | GTTTCCCTATTAGGGTTTCTAGTTAAACCAAAACTGACCAACC | | | | | |
| 151.645645 | | ATATAAAACATAACCCAAATTAACCCATTTGTATA | | | | | |
| RAD_kmer_0002161_1_TGCAGGGCAGAGCAAGGGAAGCTG | 283 | TCAACAAATCTCAACAGCACTAACCTCTGGTTTGTTCTTAA | T | Y | Y | T | GYSNP0634 |
| GTGAGATAAACAAGAGCTTGCTTACGCTTGGCAGAGTTAT | | TATTTTTTGCTCGGTGTGCATAGTCTAAAGTACTGAG[T/C]GTT | | | | | |
| AAATACATTAGTTGAACACTCAGGGCA_NODE_1_length_361_ | | TCCTCCAAAGAGTAAATAGAGGGAGAGATAGTGGCAATTAT | | | | | |
| cov_38.351799 | | GCATGTCTTTGTTTTCCCTCCCAAAGAATCTCTTAA | | | | | |
| RAD_kmer_0002163_1_TGCAGGTAATAATGCATAATCAAGC | 261 | ACAAAACAAAACCATACCATGTCATATCTACAACTCTACAAA | C | Y | T | T | GYSNP0635 |
| AAGTTATATATTTGTATTATTCCAGTAATAACGATCATCTA | | AAACATAATATAAACATTGTAATAGTAACCAGATTCAA[T/C]T | | | | | |
| GTTTCTGTTATTCATTTGAAATGAC_NODE_1_length_600_cov_ | | TCAATTTTTTAACTAACAATAATCAATCTAAAACCTATAACCA | | | | | |
| 148.373337 | | GAACCAGGTGGTGCTGGTGGCAACCGGTTTGGGGTG | | | | | |
| RAD_kmer_0002182_1_TGCAGGTATTGTCATTACTTCTATAT | 148 | GTTCATTTCTTCGTTTAAGTCATTGCTGTCATTTGCTCATTCGG | A | — | G | A | GYSNP0636 |
| CTCAGGTAGCAACCGCAAACAATTTGCAATAGCGACACT | | TGGTTTATTTTTAACAACAAATTAATTGTAAATGTC[G/A]ATTT | | | | | |
| AATGTATAGCCTATTACAACAACAGA_NODE_7_length_467_ | | CAAGGATGACCCTGATTTGCATGTCTTCCCCAAGTCGAAGTA | | | | | |
| cov_20.276232 | | TGTTGGCATAGAGCTGTGGTAGGTAATCAATTAT | | | | | |
| RAD_kmer_0002182_1_TGCAGGTATTGTCATTACTTCTATAT | 381 | CTGGAACAATGTTTCACAACGTCCTGATATACGATGATCTTG | A | — | R | A | GYSNP0637 |
| CTCAGGTAGCAACCGCAAACAATTTGCAATAGCGACACT | | AATACGCCCACAAGTCACAGAAGAAACATGCGGAAAAC[A/G] | | | | | |
| AATGTATAGCCTATTACAACAACAGA_NODE_7_length_467_ | | AAAAGACGTATGCGACCTCAGTATCGATAAAACTTATTTGTT | | | | | |
| cov_20.276232 | | TATATCATCGTTTAGGGCTGTTAAAACTCGCCATTGTT | | | | | |
| RAD_kmer_0002196_1_TGCAGGAGAACAAGCTCAATCCCAC | 446 | ACTTTGAGTTTGGAAATTAGCATACATTGAGATTTTACTTTGT | G | G | R | R | GYSNP0638 |
| TCCCCTTTTTCAGCTGATTTACTAGATACTGGAATAGACCC | | TTGTCTCACTTTAATCGACATTTAATATTATTTAGCC[G/A]AGA | | | | | |
| CTGAGTTCCATTGATGGGTACTTTT_NODE_1_length_548_cov_ | | ATAACAAACTACAAAGTGCTTCTTTGCAGGTGTAAAAAGTAC | | | | | |
| 77.350365 | | CCATCAATGGAACTCAGGGGTCTATTCCAGTATCT | | | | | |
| RAD_kmer_0002202_1_TGCAGGATTGCAACATATATGGGCC | 183 | TTCAGAAGCGCTCTCGTTTTACAACGGCCTGTACAAGAACCT | Y | T | Y | Y | GYSNP0639 |
| TTATTGACATTTGGCAAGTGGAGTTGGGGAGGACGCACAT | | TTATTAGTCGTGGCTTTTCTTCCTTTACCATCAAACGG[C/T]CT | | | | | |
| GTCCAGCAGCTAGTGGCACACACATT_NODE_1_length_416_ | | TACAGTTGGTCAACTTGTCACATTCAAATGGAGACTTGAAAG | | | | | |
| cov_57.028847 | | GTTAACCAGTTTTCACCTCGAGGACGAATTATTGTA | | | | | |
| RAD_kmer_0002203_1_TGCAGGTATGAAATATTTCTACATC | 109 | TTAACTGTTGCATGGTTGACATTTGTGATAATGTGATTGTTTG | R | A | R | A | GYSNP0640 |
| CCCTCTTTGTGAAATACCTTTATTCATACCATGTTTGCTTC | | AGAGGATAGCGCACGCATATGGTTGAAATCTCAATTT[A/G]A | | | | | |
| TCGTACACATGATCGTAACTCCTTT_NODE_2_length_447_cov_ | | AAGTCATTTATATAAACTAGTATAACAATTACTAAATTGACA | | | | | |
| 50.384789 | | TCTGAATCATTATGTATACTCTCCTCTTATACATTAA | | | | | |
| RAD_kmer_0002206_1_TGCAGGCTTTTGAGGTGTTCCAGAC | 288 | GGTTTAAGAGTCTCGTGGCTTCGGCCCCCCCAAAAAAAGTTT | W | W | A | W | GYSNP0641 |
| TCATGAGATCTGAGCTGATAAGCGGCAGACGAAGTCTCG | | TCAAGCTCCGCCACTGCTGAGAAAGATGGTTTCTTTTT[A/T]A | | | | | |
| AACAAGAGTGTCAAAATGGTTGCCACC_NODE_1_length_497_ | | TTAAGGCATTTGCGGCTGGTGTCATATTGATTATTGGCTACTG | | | | | |
| cov_189.370224 | | GTTTTATACATGTACTTCCTGATGCATTCGAGAGCT | | | | | |
| RAD_kmer_0002215_1_TGCAGGTAGATATCCCATCTTCTTTT | 403 | CACCGCAGCAATGAAGCTACACCGCAGCTCAGGAGGGATCT | Y | Y | C | T | GYSNP0642 |
| GCTGCATTGCTTTTGAAGAACTGTGAGGCACCATACCCGA | | AAATCGCATAAAAGTTTAGTCACTTCAAAATATAATTAT[C/T] | | | | | |
| ATCATGGACATGTTATTTTAGCAGA_NODE_1_length_640_cov_ | | CTTTTGGTATGGGATAGACTGTTGATGTATATGGTGTACCTGA | | | | | |
| 146.295319 | | GGAGCAACTTTTGTCTTCAAATAAAGGGCCATGTCAC | | | | | |
| RAD_kmer_0002218_1_TGCAGGCTAATGCTCCTACTGTATT | 460 | AATGATCTACGCAAAATTGAGGATCATCCATACCCACCTAAA | C | A | M | M | GYSNP0643 |
| TTCACTTCTACTATCAGAGCCCCAACCCGAGGTTCACTAA | | GGGTTGGGTTTAGAATACCATTCACATTCCTATCCAAT[A/C]A | | | | | |
| TCATTACACTCTTCGGATAGGGTTTT_NODE_1_length_597_ | | TTTATCAAACCCGCAATCATAGTGATTCCCTGTTGCAAACCAC | | | | | |
| cov_83.597992 | | TCGCCACCTGAATTAGTAATTGTTGACAAAAACCCT | | | | | |
| RAD_kmer_0002224_1_TGCAGGTGTGAACTAATTATCAAAA | 415 | GGCATTATGACATCAACAAAGGGTGTATCATTTTTGTGTTCA | G | R | R | G | GYSNP0644 |
| TGAAAATTAAATCAATCATGGTTTGTAAAATATAGCTAAC | | AAATAGGGATGTTCTAAATCTTCCAACTGGTTATATTC[A/G]A | | | | | |
| CAAGTTTTTGACAAACCATTTTTCTT_NODE_1_length_617_cov_ | | ACACTATTGACTATTTTTTGGACTTAGGACACTTGACCCCTTT | | | | | |
| 86.338737 | | ACTTTCATTGTAACTAAAAAGAGAGAAACGTTATAT | | | | | |
| RAD_kmer_0002227_1_TGCAGGTCTTCCAGCTCTCGAGTGC | 214 | ATACATTGGGTTTCATGGGACCGGGTTACTTTAAGTAAAAAG | C | Y | Y | C | GYSNP0645 |
| AGGCTTCATATAAGGGATGATCCAGAAAAATGTTCTGAGT | | GCGGGAGGTTTAGGGATCTCGAGATTGAAGGAAGTGAA[T/C] | | | | | |
| ATTCCAATATTGACCCCGTATAAGAC_NODE_1_length_420_ | | GCTGCTCTTTTATTCAAATGGAACGGTAGGTTTCGCTCGGAAT | | | | | |
| cov_38.219048 | | CCGGTAGCTTATGGAACCGAGTCATGAGTTATTGTCA | | | | | |
| RAD_kmer_0002227_1_TGCAGGTCTTCCAGCTCTCGAGTGC | 274 | TCGAGATTGAAGGAAGTGAATGCTGCTCTTTTATTCAAATGG | A | M | C | M | GYSNP0646 |
| AGGCTTCATATAAGGGATGATCCAGAAAAATGTTCTGAGT | | AACGGTAGGTTTCGCTCGGAATCCGGTAGCTTATGGAA[C/A]C | | | | | |
| ATTCCAATATTGACCCCGTATAAGAC_NODE_1_length_420_ | | GAGTCATGAGTTATTGTCATAAGACTCGGGCTAGTTTCTACTC | | | | | |
| cov_38.219048 | | CTTACCGGTCAACAATCAGCTTGGCGTGAACCGAAA | | | | | |
| RAD_kmer_0002227_1_TGCAGGTCTTCCAGCTCTCGAGTGC | 326 | TTCGCTCGGAATCCGGTAGCTTATGGAACCGAGTCATGAGTT | R | G | R | R | GYSNP0647 |
| AGGCTTCATATAAGGGATGATCCAGAAAAATGTTCTGAGT | | ATTGTCATAAGACTCGGGCTAGTTTCTACTCCTTACCG[G/A]T | | | | | |
| ATTCCAATATTGACCCCGTATAAGAC_NODE_1_length_420_ | | CAACAATCAGCTTGGCGTGAACCGAAAATGTGTTGTGAAATT | | | | | |
| cov_38.219048 | | TATTGACAGCCTTACGGTGGGAGAACGCCCTCTTTTC | | | | | |
| RAD_kmer_0002229_1_TGCAGGAAAGAGTCTTAGCATAATA | 466 | CTTCATTTCAGAAAATATGATGAGCGTGTTGGAAAACGCTGA | C | C | T | Y | GYSNP0648 |
| CAGGTGAGATACATAAAATAAGAAATTAGATTGTTAAAA | | CATGGGCATACGATTCCCTGATCTTCAGTTTTTGGCAA[T/C]G | | | | | |
| TCAACCTGAGTTATGGAGACAACATAG_NODE_1_length_648_ | | GCATCGTCTCCTGTACTTGCATCTGATGCTTTTTCAACTTTAA | | | | | |
| cov_90.186729 | | TGTGGATCCTCTTTTGTCTTCCAGTACCTTTTATGT | | | | | |
| RAD_kmer_0002247_1_TGCAGGCCACAAATTATTCACAAAT | 257 | TCATTCAAGGGTAATGTTAACTTGTTATGTGTTTATAAAGATT | A | G | G | G | GYSNP0649 |
| CAAATCATAAATTCTAGTTATTCATAATTAGTTTTATGGAC | | TATGTTTTAATGGATTTCTATCTTGGTTTAAAAGGGC[G/A]CG | | | | | |
| GCAAGGCGCTCCAAGGCGTTTAGGT_NODE_1_length_618_cov_ | | CCTTATGCACGAGGTGCTCTAGGAAATTAGGAAAGTGCCTTT | | | | | |
| 129.991913 | | AGGTCCTTGAGGCACCGCCTCATGCACATGTGGGTC | | | | | |
| RAD_kmer_0002249_1_TGCAGGAGTCAGCAACATCTTGCAC | 217 | GCATTGAAGAAACAGCTTATCATCTCCACTGTTCTTATGACCG | — | G | K | G | GYSNP0650 |
| AGGGCTGCATACATTAGAAGATAAATGAATAAATGTTCA | | TTGGCATTGCAATCGTTAGTTTCATTGGGCTCCCGAC[G/T]TCC | | | | | |
| CATAAATTAAAAAAATAAAAAATAGAA_NODE_1_length_548_ | | TTCACAATCTTTAATTTTGGTGTTCAGAAGGTCGTGTATAACT | | | | | |
| cov_77.029198 | | GGTAAGCAAAAGTTTCTTATCGGTTCATTTTTAT | | | | | |
| RAD_kmer_0002249_1_TGCAGGAGTCAGCAACATCTTGCAC | 351 | AAAAGTTTCTTATCGGTTCATTTTTATATATACACTGTTGAAA | C | C | T | C | GYSNP0651 |
| AGGGCTGCATACATTAGAAGATAAATGAATAAATGTTCA | | TCACACCAAGTTTATTAATTTTGGTGTTTACTTTTGA[T/C]GTC | | | | | |
| CATAAATTAAAAAAATAAAAAATAGAA_NODE_1_length_548_ | | TGTATGCAGGCAACTTTTCTTGTGCGTATGTGTCGGTCTTTGG | | | | | |
| cov_77.029198 | | GCTGGACTAATCATTGGTTTTGTGACCGAGTACT | | | | | |
| RAD_kmer_0002250_1_TGCAGGACCGGTCCGGTTTTGATCA | 102 | CTCTGTTCACCAAATACAAACCTGGTGCATGAGGCTCTCGCA | M | M | M | C | GYSNP0652 |
| TAGAGTGTCATTTTCCAATCCAACATGTTCATGTCTGCAGT | | TGTGCGTGTCGGGCCAACGTGGGCCTTATGCACGCATC[A/C]T | | | | | |
| GGGCTCGGTTAATATAGGCTTGGGT_NODE_1_length_466_cov | | TACCTTGCATTTCTGATGGAGGCTGTTTCCACTAATCGAACTT | | | | | |
| 209.618027 | | GCAAACCTCTATTCGCTAAATATGTAATTAAATGAC | | | | | |
| RAD_kmer_0002251_1_TGCAGGTTGGTGATAGTAACACTAC | 302 | ATCTAGGGTACATGCATTATTGTTTAGGGTACGACATTAGGG | R | R | A | A | GYSNP0653 |
| AGCCATAATATAGCTTCCAAGATTTCAGTTTAAGATAAAT | | CTGAAGAAAGTATCAAGTATTGATTGGTCCCACAATGA[A/G] | | | | | |
| ACCATCTCTAGTTTGTGTTTTATTTC_NODE_1_length_624_cov_ | | GGTACGTGTGAAAGCTCTATGAACACAATGATACAAAATGAA | | | | | |
| 77.451920 | | ACCAGGCAATTGATTACTGTAAAACCATGGAGGAATAA | | | | | |
| RAD_kmer_0002257_1_TGCAGGATCAATCAAGCTATCGAGA | 204 | ATATTTTTAAACCAAAATATAACATTTAGTTTCTTTTTCTAAT | A | C | A | C | GYSNP0654 |
| TTTGATTTGAGAAAAACCGTCCTTGAATATTGCTTCCACG | | GAATTACAGCCGTGGTCGGGGATGGATTTATCGGCCG[A/C]G | | | | | |
| GTCTCCCGAGGTATGTTTTAACTCCG_NODE_1_length_385_ | | GTTTCACTGTCCGAAACACCGCGGGCCCACAAAACCACCAAG | | | | | |
| cov_30.381819 | | CAGTGGCGGTCCGCAGCGGCTCGGATCTTTCGGTATT | | | | | |
| RAD_kmer_0002259_1_TGCAGGTGAGGAACGTCTTCATTTT | 290 | TCCTTTTGATTAGACATATAATGCTTCAAAAGGTTGTCACCTT | A | — | A | R | GYSNP0655 |
| TTTCTTTTAATTTTGTCCGTTTGTGTTTGAAGATTATATAC | | CATGTTGAAATGGACAGTGTAGCAGATTCGGGTGTTC[A/G]TC | | | | | |
| CTTAGTCTAATTTCAAGTATATATA_NODE_1_length_400_cov_ | | TTCTCTATAATTTTTATTAATTTTCCCAACACCTGATGAATAG | | | | | |
| 67.224998 | | TATATATACTTGAAATTAGACTAAGGTATATAATC | | | | | |
| RAD_kmer_0002267_1_TGCAGGATTGAGAATGAACGTACCA | 143 | GAACTTCAGAGCTTTATTGACAAGTAAGTAATTAGATGTTAG | A | C | C | C | GYSNP0656 |
| AATTTGGTTATGTACAATAGGCTACATAAATAAATGCACA | | TCAGTTAAACCTGTTACTGCTAAATATTTTTTTTACCT[C/A]CA | | | | | |
| GACAATACTAAAAAAATAACATTTTG_NODE_1_length_685_ | | TATACATATGATTTAATAACTGGTGTTGACTTATTAGGTTAAA | | | | | |
| cov_72.191238 | | AGTCTGGTAGCAATGACAAATAAGTGCATGTTTGG | | | | | |
| RAD_kmer_0002278_1_TGCAGGCAGATTGTGTAACCTTCTT | 165 | GAAAAATTATGCTGGATTAATTTATAAGTACTTGCTATTTGTT | C | C | A | A | GYSNP0657 |
| CAGGCTGTTGACATTGAATGAACGGAGGGATGCATCCCTC | | TTTGTTAGGTTCTGGGATGACATTGAGGAGTGATGAC[A/C]CG | | | | | |
| ACTTCCAATTATGCGGATCCAAAAGA_NODE_1_length_554_ | | GCCATCATGAAAGGCTCCAGAACAAGGTGACATAAGTGAAG | | | | | |
| cov_84.070396 | | ACTACATCTGGAGATAGCCAGGGAAAAACTATAAGTA | | | | | |
| RAD_kmer_0002283_1_TGCAGGGGGTTATCTCTTTGATTTTT | 227 | TGAACGATGTGATGAACGATCTTTTCTGGGACCATTTTCTGCT | R | R | R | G | GYSNP0658 |
| GACAAAGCTGTTCTCGAGCCTACGTTTTGCCCTATGTATG | | TCAAAAGCTCACCAATTAAACGGATGTTTCCGAGAGT[A/G]C | | | | | |
| CTCAACTGTGTTCAGATCTCAACAA_NODE_1_length_475_cov_ | | GCAGCTTAATTATTCTTTCTTTATCTCTGCGTTCAGATTCTTGT | | | | | |
| 160.035782 | | TCAGGGGCAGTCATTTGCCTAATTTCTGCCCTTAG | | | | | |
| RAD_kmer_0002288_1_TGCAGGTCCAGTTTGTTCAAAACCT | 377 | TGGAAAATGAATAATTAGATTATTTGATTACAAAATATTATG | W | A | W | A | GYSNP0659 |
| CAATTAGTTGGAAATTGTGGTTCTTATACCTTTTGAGCTTT | | AACTGATAACTGGACTGGATGAGTATAAAGTGACTATT[T/A]T | | | | | |
| TCGGCACATCTGATGTATTAATTTT_NODE_1_length_436_cov_ | | TTATAAGGAAGTGAATGAGAAGCTACTGGATGCATTGTCATC | | | | | |
| 49.348625 | | AAGCTCACTCATTGTGGATAAACGTACATATGAATTT | | | | | |
| RAD_kmer_0002290_1_TGCAGGAACAGGAAGACGACGAGG | 386 | ACAACGATCTCGCCCTCATCTTCTTATTCGAACCATCATCATC | — | Y | T | T | GYSNP0660 |
| AGGATATGGATGTTCGAGGATTTAACCCCGCAACAGCAAA | | CACATCCATGTCATGATCAGCTGACCTCTCCCTCTTA[T/C]GA | | | | | |
| AGGAGCTAGCGGAAATCAGGAGAAAGAA_NODE_1_length_ | | CCTCTAGACTTGCTTCGAGCCCGTTCAATTGCTTTAGACGGGT | | | | | |
| 630_cov_97.315872 | | CAAGTCCCAAAGATGAAAGTTGTCGGCCCATTCTC | | | | | |
| RAD_kmer_0002290_1_TGCAGGAACAGGAAGACGACGAGG | 490 | CGTTCAATTGCTTTAGACGGGTCAAGTCCCAAAGATGAAAGT | — | R | A | A | GYSNP0661 |
| AGGATATGGATGTCGAGGATTTAACCCCGCAACAGCAAA | | TGTCGGCCCATTCTCTCAGAAGTGAACTTTTTGTCCTT[A/G]TC | | | | | |
| AGGAGCTAGCGGAAATCAGGAGAAAGAA_NODE_1_length_ | | GAACTTTCTTGGTACAATGGGCCGGCTTTCTGCTGTGCTTTTC | | | | | |
| 630_cov_97.315872 | | TTAATTCTATGTTCTCTAATGAGTATGCTTTTCTT | | | | | |
| RAD_kmer_0002292_1_TGCAGGAGACATGGATGATTCGTTT | 180 | AAGTAGTTAAAAGCTAAAACCACACTCATGGTAGGGTCCGTT | T | A | A | T | GYSNP0662 |
| TCCCGAATTCAAAATATAAATAACTTGTTATACATAACCA | | GTGCTTTTAGTAAACAGTTACAAGACTCTAAAAGTAGG[A/T]A | | | | | |
| ACCATGCCTATGTGTCTTATTCCAAA_NODE_2_length_302_ | | TCTTGAAAGCAATCTGGTTTATTCTTCATTCTGCTTTAATTGTC | | | | | |
| cov_29.112583 | | TTCTTTTTCTTTAATTTTTTAGTTTAAATGATACA | | | | | |
| RAD_kmer_0002300_1_TGCAGGAGATTCAAGGAGCTCTACA | 397 | TATCAATCTTTACATGTCACAATACAATGACAACTTAAAACG | A | G | G | — | GYSNP0663 |
| AGTGAGAAATTGAACTTTCGGATTTCAATCCAGGTTTTGT | | ATTTAGATACGCATAGAAACACAATAATTTTAAGCTCT[G/A]C | | | | | |
| AGTTTTAAACCTTTCAATAAATTTCG_NODE_1_length_526_ | | AGAAATCAAATATCTTGACAAATCTATGGCTCCAATTTGTTA | | | | | |
| cov_130.604568 | | AAAAGAGAGTCGAATGCAACACGAAATTTATTGAAAG | | | | | |
| RAD_kmer_0002302_1_TGCAGGTGACGAGTTACCAATGTCA | 191 | ATTGGGCCTTTGGGCTTTAAAAGCAGAACTTGAAGACCTCTG | R | R | R | G | GYSNP0664 |
| TATTAACATTCCAGATATGTTTTTTCATTATTTATAGTCTT | | CTTTGCTGTTCTTCAGGTCAGTGATTACTACTGATACC[G/A]TG | | | | | |
| ATTTGAGAGTTGAAGGTTTATGACA_NODE_1_length_501_cov_ | | TGATCATTGTATAGAGATGAATAATTACTGATTTATATGGCGT | | | | | |
| 190.814377 | | GCAGCCTCAGATCTTCCGCCAGATGCGATCTGACC | | | | | |
| RAD_kmer_0002309_1_TGCAGGCTGTAGTGCTATATCAAAT | 242 | AGGATCATTTAAAGGATATTTAGATTTTTTAAAATTGTCTTAT | C | Y | Y | Y | GYSNP0665 |
| CTACACCTAAGTAACCTAGAACCGTCAAAATACTAGTAGT | | TTTGTTTTTAAGTATTCGTATTTTATTTTTATAGTTT[T/C]AGTT | | | | | |
| ACCTAGCATGATGTACCCTAAAACTT_NODE_1_length_396_ | | GTGTAACATTTCTAATTTCTAATTTTAATGAAATTTAAATTTT | | | | | |
| cov_44.515152 | | AATTAGATTTAATTGTTTAAATTATTTTAAATT | | | | | |
| RAD_kmer_0002316_1_TGCAGGCTGCATCACACGCATGCAC | 244 | AGATCTAGAGTTCATTGATGAGTTATTTGCAAATGTTGGAAG | R | G | A | R | GYSNP0666 |
| CAAGTAAGGTGGACCAGAATGAATCACATTGTTACAACA | | ATAGTTTTTCTTGCTCTTCTCGAGCCTACAGAAAGCTT[A/G]TG | | | | | |
| ACTGCAGTTTAAATAACCTGAAAACAT_NODE_2_length._359_ | | TACAAAGAGTCAAATACTGTACCTTGATTGACCGTAGTTAAA | | | | | |
| cov_39.724236 | | TTACTAGTCAACTGTGCACTTCCATTGGTTTAAATT | | | | | |
| RAD_kmer_0002319_1_TGCAGGTGATGTTGATGAGGAGGA | 381 | GCACCAATACCTTGAATTTCTCTCTTTAGTAATCGGACTGGGC | W | T | W | W | GYSNP0667 |
| GAAGGCAATTGTCCTATACAAGGTAACAAAACACTAAGT | | AGCTCATGGGCACCAGGCTGACAGAGAAAAAAAAAAT[T/A]G | | | | | |
| AGAGTATTCTTATATTCTAGTGTGTAAT_NODE_1_length_464_ | | AATCAATAATCGATTTTATTACACACTAGAATATAAGAATAC | | | | | |
| cov_92.364227 | | TCTACTTAGTGTTTTGTTACCTTGTATAGGACAATTG | | | | | |
| RAD_kmer_0002330_1_TGCAGGCTGCAACATTTTTCACTCC | 556 | GTGATTAATATGTCTTTCATGTTTTCAATGTGTGAAGTGTCGT | C | M | C | A | GYSNP0668 |
| AATTGTTATCTCAGTGAGATGTATGAGGCGGTACACCACT | | TTCGAGTCTTGGAGATTTTGTTATATACTTATCGGTA[C/A]TCT | | | | | |
| GATCAATGAAATCTCAAAGTGAAGAG_NODE_1_length_620_ | | TCACTTTGAGATTTCATTGATCAGTGGTGTACCGCCTCATACA | | | | | |
| cov_148.848389 | | TCTCACTGAGATAACAATTGGAGTGAAAAATGTT | | | | | |
| RAD_kmer_0002331_1_TGCAGGCAATTATTTACAAAATGAA | 338 | ATAAAAAACTATAGCTACCAGCTATCAGCTACAGCTACCAGC | Y | C | C | T | GYSNP0669 |
| TAAGTCACAAAAGGATCGAAATAGAACAAAAGATGAAAC | | TACCAGCTTCCAGCCACCAGCCAACAGCTTCCAGCCAC[C/T]A | | | | | |
| TAACGGACGCTCACACTATGCTCATAA_NODE_1_length_460_ | | GCCAGCAGCTAGTTTTCCCAAACATACCCATGGTTGACCTTG | | | | | |
| cov_60.582607 | | AAAATTGTATTATGCATAGAAGCTGGCAATTCCTTAA | | | | | |
| RAD_kmer_0002343_1_TGCAGGACCGCTTCTTATACCACGT | 163 | ATATGCATATGGAAACATAGCACATCTTATCATCATATTTGAT | W | T | T | W | GYSNP0670 |
| AAAGCTATTGAAACATGTGAAGTAATGGGATACACCATTC | | TACAACTTGTATAAAAATGTTTTAAGTTACATGAAAC[T/A]TG | | | | | |
| CAAAAGATAGTCAAATATTAGTTAAT_NODE_4_length_353_ | | TATCAACTAGATGATATAAACCTAATCCATGGTGTCAATTCTT | | | | | |
| cov_40.311615 | | GAACTGTCCATTACTGAGCTTGAATATTACACGCA | | | | | |
| RAD_kmer_0002343_1_TGCAGGACCGCTTCTTATACCACGT | 239 | AAACTTGTATCAACTAGATGATATAAACCTAATCCATGGTGT | R | A | A | R | GYSNP0671 |
| AAAGCTATTGAAACATGTGAAGTAATGGGATACACCATTC | | CAATTCTTGAACTGTCCATTACTGAGCTTGAATATTAC[A/G]C | | | | | |
| CAAAAGATAGTCAAATATTAGTTAAT_NODE_4_length_353_ | | GCAGTGGTTCCTTCTTGTACATTGCTATATCCATCTCCTCATC | | | | | |
| cov_40.311615 | | CATGTTGATAGCCATGGGGTTTATATTATCCGGAAT | | | | | |
| RAD_kmer_0002347_1_TGCAGGTTCAAAATCCATGTTTCAC | 117 | AATGATATATTTTATATGCTAAAATTAACGCATCAATTACTCA | C | C | T | C | GYSNP0672 |
| GATCACTAAACTGATCGCGGGCTCAAACGTATACGCTTGC | | ACGAGTAATCCCCGTGGGAGTACTCGGCGTGTCATCT[T/C]CG | | | | | |
| CAGCTAAAGTCCAGCACAATAAGGCT_NODE_1_length_399_ | | AGTACTCCCCGACTATGGCCGAGTACTCCCAACTCCCCTGTC | | | | | |
| cov_104.837090 | | AACCGATTTAGCACCGTCTAGCGACTTTTGCAACCA | | | | | |
| RAD_kmer_0002347_1_TGCAGGTTCAAAATCCATGTTTCAC | 280 | GTAATAACATTGCTAAAATCCAAGAAAAGGGGGATACTAATT | Y | C | T | C | GYSNP0673 |
| GATCACTAAACTGATCGCGGGCTCAAACGTATACGCTTGC | | TCTTACAGTTTTAAATGCATCCAAAGCAGCTCCAGAGC[T/C]G | | | | | |
| CAGCTAAAGTCCAGCACAATAAGGCT_NODE_1_length_399_ | | ATTGAAATTTATAATTCTCCAAGATCTAGAAAGGCCTATACC | | | | | |
| cov_104.837090 | | ATAGTTGATCTAGCCTTATTGTGCTGGACTTTAGCTG | | | | | |
| RAD_kmer_0002352_1_TGCAGGAAGTAGGTCCCAGTCATCG | 205 | TCAAAGAGAATGTATGCCTTCATGTTTAATTTATTTACCTGAT | T | T | Y | Y | GYSNP0674 |
| AGAAGTTTAGGCAAAAAGAAAGCTCTTATATCTCCTAGTT | | ACCAAGTTGCAGATTAAGCATCAGATAGTGGCTTTTA[T/C]GG | | | | | |
| CTTCCTTTAGGTCAGAGAGGTTTAAG_NODE_2_length_440_ | | TCTTCAAAAATGCCTGCGGGCTTCTTTTTAATTTCCTTCATTTG | | | | | |
| cov_53.572727 | | AAATTGCGTTGTTCTCTTTTATATGATGATCCA | | | | | |
| RAD_kmer_0002352_1_TGCAGGAAGTAGGTCCCAGTCATCG | 309 | TTCTTTTTAATTTCCTTCATTTGAAATTTGCGTTGTTCTCTTTT | T | T | K | K | GYSNP0675 |
| AGAAGTTTAGGCAAAAAGAAAGCTCTTATATCTCCTAGTT | | ATATGATGATCCAGCATAATTCTTAGTCATTTCTTT[T/G]ATCA | | | | | |
| CTTCCTTTAGGTCAGAGAGGTTTAAG_NODE_2_length_440_ | | GAACCCCTTGTATATATTCCCTTTCACAAACTATAGTACTATT | | | | | |
| cov_53.572727 | | ATCATCTTCTTCACTTTCTCCTTCATCAGATAA | | | | | |
| RAD_kmer_0002353_1_TGCAGGTAAACATGAAACTGAATG | 293 | TGCTAGAAAATTGAGTTTGTGACTGCATTTGCGGTCGCAAAA | G | T | T | T | GYSNP0676 |
| AAGAGCGGCAACACTTTCTAACCCACAAGACGCAAGATA | | TGGTCACAATTGCCTTATTGCGACTGAAAATGCTGTAG[T/G]A | | | | | |
| ATACCTCGAAAACTAGAGAGATAACATA_NODE_1_length_ | | AATACACCGTCGCAATTGCCTTATTGTTACCGCTTCTATATTT | | | | | |
| 595_cov_106.448738 | | TGTGACCGTTCCTTTTTGCAACTCAATTTTGTTTTG | | | | | |
| RAD_kmer_0002353_1_TGCAGGTAAACATGAAACTGAATG | 401 | TTACCGCTTCTATATTTTGTGACCGTTCCTTTTTGCAACTCAAT | C | C | T | T | GYSNP0677 |
| AAGAGCGGCAACACTTTCTAACCCACAAGACGCAAGATA | | TTTGTTTTGAAAGGATTTATTTTAGTTTCCAGTGTC[T/C]TTTC | | | | | |
| ATACCTCGAAAACTAGAGAGATAACATA_NODE_1_length_ | | GATTTACAAATATATCCAAAATCATATATAAGAAGACGATCA | | | | | |
| 595_cov_106.448738 | | GACCAAAGATTTATATATATTGACGAAATTATAC | | | | | |
| RAD_kmer_0002356_1_TGCAGGAACATATAACTACCCTATA | 321 | TTCAAGTACACGTCACCCGGTTCATATCTTTATCACACTATAC | A | A | W | W | GYSNP0678 |
| GATTCACTATGTATAATATATGCCACAAACGGTCCGTTGT | | AGTTGCGTGATACCAAACAGAGTGTCATGAACTCCCC[T/A]TT | | | | | |
| CCTCTGTGGAGGTTTCCCTAACCCCT_NODE_1_length_582_ | | TTGTATTTTAAATATTTCGGGGTGAGCACATGCTATGTTTTAT | | | | | |
| cov_173.087631 | | TTAAACTTTTGTTATGCTACAAGATACTGTTGCCA | | | | | |
| RAD_kmer_0002357_1_TGCAGGGTCAGATAAATATGCATTT | 402 | TTCTTATTATGGCATCTGCAGCTGGGTAATATATTTACTTCCC | Y | T | Y | Y | GYSNP0679 |
| TGCCATTCAATTTGAGTCCACTGATGGCTCAAAGGCTCGA | | TACCTTTTAGTTTACATGCTTGATGGTCAATGCTGAT[T/C]TCA | | | | | |
| GCAACCAGGTCATAACTTCTTGTTGT_NODE_1_length_506_ | | TTGACCATGCTATTTACACTGGTGCAGACCTCAACAACAACA | | | | | |
| cov_178.144272 | | AGAAGTTATGACCTGGTTGCTCGAGCCTTTGAGCC | | | | | |
| RAD_kmer_0002361_1_TGCAGGATATGAGAGTGATGGACCC | 116 | CGCCGACTAACGTCCAAATCAGTAAAGACATTAGTGGGTAAC | G | R | R | G | GYSNP0680 |
| ACAACATCAAATGAGATGACCCCATTGTCTTTTACAATGT | | ACATGTTCTTATAGTTTAACTTCATCAACATTGACCAT[A/G]T | | | | | |
| TTTTATGAGTAAGTAATGTCGAGAAA_NODE_1_length_610_ | | ATTTTAATTCATATATATACATATATTTTCTAACTATCATGAG | | | | | |
| cov_75.060654 | | CTCCAGAAATTTAATGTTGGAAAATAATTATTTAAA | | | | | |
| RAD_kmer_0002361_1_TGCAGGATATGAGAGTGATGGACCC | 181 | ATCAACATTGACCATATATTTTAATTCATATATATACATATAT | A | R | R | A | GYSNP0681 |
| ACAACATCAAATGAGATGACCCCATTGTCTTTTACAATGT | | TTTCTAACTATCATGAGCTCCAGAAATTTAATGTTGG[A/G]AA | | | | | |
| TTTTATGAGTAAGTAATGTCGAGAAA_NODE_1_length_610_ | | ATAATTATTTAAAAAAAAAGAAAGATATTATAGGTCTATTAT | | | | | |
| cov_75.060654 | | TATTGTGATTTTTAATGTTTATTATTTTTTAGTATA | | | | | |
| RAD_kmer_0002363_1_TGCAGGTCACACAAACAATTACATT | 252 | CAGAATATAACAGATCTCTTGCCAAGGCTTTGAGTGGCAT | A | A | G | G | GYSNP0682 |
| TTAATAAATCACCAAAAAAATATGGATTTTTAACAAGATT | | TTTAAAAAAGTATTACTCTATTGTCTTTTAATATTTTG[G/A]TT | | | | | |
| CAGGGTAAATAATATTGCCTGTATGC_NODE_1_length_545_ | | TGTTGAATATATATTATAGGTTATATACTTATTCTTTGTAGGC | | | | | |
| cov_135.677063 | | ATCTCATGTTGTTTGAAGACATTGGAGGCGATTAC | | | | | |
| RAD_kmer_0002364_1_TGCAGGTGCTTTAAAACTATAGTAA | 91 | TCTGAAGATCTATATAAACCGAAAGACTTGTTCGATCCTGGT | W | T | A | T | GYSNP0683 |
| AAGGAGGAGGGCTCCTTAATTGAAGAGGGCCCCACGAGT | | GATTCACTATTAGCATCGGTATTCTCTGGTGAAGTTCA[A/T]A | | | | | |
| ACTCAGGTGGGACCATGCTTTGTTTGG_NODE_1_length_592_ | | AGTTTCCTGGTGAAAGGTTTGTTTCAGATGGATGGCTTAACAT | | | | | |
| cov_87.266891 | | ATTAAGAAAAGTGGGCCTTCAAAATGCTGGTGATCC | | | | | |
| RAD_kmer_0002365_1_TGCAGGAAGCATTCACGAAGACAG | 133 | TTAAAACATTGGCGCTAAAAAAGAACTGGGTCAAATGGGAG | T | — | A | A | GYSNP0684 |
| CCCAGCTCCAAAATACCTTCTTGAATCCGGGGTTGACCGC | | ATACACATCTAGCATACAACTATAAATAAAAAAATTAGA[A/T] | | | | | |
| AAACTCTTAAATTTTTATGGTAGTAGA_NODE_1_length_568_ | | CATACCTATTGTTCTAACTTCTAACTACTTCCAAAGTGAGCA | | | | | |
| cov_96.121475 | | TTAGTAGCCTAAAAGATCCAAATGGATCCCAAAAATTA | | | | | |
| RAD_kmer_0002367_1_TGCAGGACAGAGGATCCATTGGGA | 195 | AAAGCACTTGCCAATGCTGCTGCGGAAGTCTCTTGGGTTAAA | R | G | G | R | GYSNP0685 |
| ACTCCGATCTTGAAATGGATATGTAGAAAAGGGGAGCGT | | AATCTACTCGGTGAACTTGGCATCAAAAACATCAAAAC[G/A] | | | | | |
| GGAAGTGGTTTTGTCAAAGCATCGGCCA_NODE_1_length_312_ | | CCCAAACTATTCTATTCTGTGACAACACGGGAGCTACATATTT | | | | | |
| _cov_63.083332 | | ATGTGCGAATCCTGTCTACCACTCCCGTATGAAACAT | | | | | |
| RAD_kmer_0002368_1_TGCAGGCATAAGAACTTGCCAAGG | 222 | CCTATTTGCACTGCCATTTTGGAAAAGCCAGATAACCGCTATT | M | M | C | C | GYSNP0686 |
| AAACACAATCACCAAAAACCAATGAATCAAACTTCTTTAG | | GACTGCTTAGCACAAAAATTAAGCAAATAGTAGAGGT[C/A]A | | | | | |
| AAACATGTACCTTTGAAACAAGTAAAG_NODE_2_length_408_ | | AGTGGCACTGGAGAGTGAAAGTTATGGTTATTTAACTGGCAA | | | | | |
| cov_37.036766 | | GTTAAGAAAAAGAGCCTAACGAAACCTTAAAAAAGAT | | | | | |
| RAD_kmer_0002374_1_TGCAGGTTAACCTGTACACAGACGC | 312 | AAGTGTTACAAGTAACCGATTTACTAGAATATGGGTAAATAT | A | W | A | W | GYSNP0687 |
| TGAGACTCTAAATATGATGGGTAAGCTGGATACCGGTTTT | | GATACATTTAGTGTTTTAAAAGGTAAATATGATATTAT[A/T]A | | | | | |
| GTAATGTGTAACTTTAGGTTTGTTTT_NODE_2_length_454_cov_ | | AAAAAAAGAAGGATATATATGAATTTGGTCGAATAAAAAAC | | | | | |
| 56.519825 | | TAGACATCAATAATGGGTATACTGGATAATCAATCACC | | | | | |
| RAD_kmer_0002383_1_TGCAGGGAAGATAGATAATATGTTC | 200 | AGCCGAAGAGATTTGAATCTCGGTTGTGAACCTCAGTTACCA | C | — | A | A | GYSNP0688 |
| CCTCAATTACTGCATAATATATTTGTACAATGTGAAAAAG | | TAGTTTCTAAAAACTTGAAAAACAATACTTTGGAATGT[A/C]C | | | | | |
| CATGCACATTGGGATCTTGCTGCTGA_NODE_1_length_582_ | | AGCACTTTTGAAATACAACTGTGACGAGTTAACATAATTTCA | | | | | |
| cov_77.355667 | | GAACCTTCTAATGGTTACTTTGGTATTTTGCTATTTT | | | | | |
| RAD_kmer_0002385_1_TGCAGGGCAAACCGAGTAGAGGTTT | 469 | TACGTAAAAATTAAAATTCCTAAATCAAGCAGAACTCGTCTT | R | A | R | A | GYSNP0689 |
| GATGATAATTTGTTACTTGGAATCACATAAAAATTAGTCT | | GACCCGTTACCACCCGACCAGCCAATAAAACAAACCTC[G/A] | | | | | |
| CTAGTAGAGGTGGTAAGACGAGGTTT_NODE_1_length_526_ | | TCTTACCACCTCTACTAGAGACTAATTTTTATGTGATTCCAAG | | | | | |
| cov_110.321289 | | TAACAAATTATCATCAAACCTCTACTCGGTTTGCCCT | | | | | |
| RAD_kmer_0002393_1_TGCAGGTATGGCCAAAAGAACCAA | 144 | AAATCCACTCAACCACAAAACAAACCAACTTTTTTAATTAAA | W | W | W | A | GYSNP0690 |
| AGAGAGATTCAATAATTGAAAAGTGGTGTAATGAGATGA | | AAACAATTCCTAAAATGGTGAATATTCATGGTAAAAAA[T/A]T | | | | | |
| CTTGTTTAAAGCAGACAAAGGAATGAAA_NODE_1_length_ | | AAGTGACTTTGCTAAAACTACTTCAAGAGCGTCAGCTTAGAA | | | | | |
| 636_cov_81.924530 | | GCTTAAAAACGCCTCCATGTCATCTCTGTTTTATACA | | | | | |
| RAD_kmer_0002394_1_TGCAGGGATTGTGTGCTGCTTTCAG | 200 | TCAGATTATCAAGTGATTCAAACTTTGCAAGAAACGTAGCTG | T | W | T | W | GYSNP0691 |
| AAAGCAAAAATACAGGAAAATCATAAAGCCCCCACATAA | | GTGTATTAGTAGAATCAGGAGCACAAAATCAAACCCAT[T/A] | | | | | |
| TACCAGATCCCTGCACACACATAGAAC_NODE_3_length_432_ | | TAAAAGGTAATAATATGGTAATTTTGTTACTTGTTAGTTGTTA | | | | | |
| cov_44.891205 | | CTTACTTTTAGATTGGTTAGAGAAGAAGAATTAGTTC | | | | | |
| RAD_kmer_0002405_1_TGCAGGTATAACTCCTAGCCATC | 345 | CTTCCTGTTTACTTTTTGTAGGCTTTTTTTTTTCTTATCGGGTT | G | K | G | K | GYSNP0692 |
| TTGAGTGCCGCTCGCCAGTCTTCTCTTTCAAGCAATCCATT | | ATTTGTGACCTCAGAAACTAAATACCAGCTTCATGC[G/T]AAG | | | | | |
| AACAACCTACTAGAATGTCCAACCA_NODE_1_length_567_cov_ | | GAAGTTGCTCATGATTTGGATCTTACAAAGTATGACGGCATT | | | | | |
| 141.194000 | | CTTTGTGTTAGCGGAGATGGGATTTTAGTTGAGGT | | | | | |
| RAD_kmer_0002407_1_TGCAGGGCTTTGTAGGATGGCTTGT | 198 | CAGTTCTCAAGAAACCGTCATACCTCACGGTTGAAAACCTGA | R | R | R | A | GYSNP0693 |
| GTTTGTAGTTATTTAAGGGACATGTGTTTAAGTGATCATTT | | GCCGGGAACCAAAACTTTCCAGTCTCGAGGGCTCGATA[A/G] | | | | | |
| GTGGGAAAATCATATGAAGAAAAGA_NODE_1_length_517_ | | CAAGACACAATCGAATCAACGGATGGTTGAGATGAAACCTTC | | | | | |
| cov_148.172150 | | ATCTTCTTAATATCATCATCATCATGTTCAAGATCATG | | | | | |
| RAD_kmer_0002410_1_TGCAGGATCAATTGTCAGTTGTCAT | 87 | TAAAAAATTTATATAAAAAACATAGTGCATATTTTATTAAAC | G | T | T | T | GYSNP0694 |
| AACCACAAGAGACTCAAAACACATAGGTTCACAAACTTA | | AGGTTAAATCGTGTTATAGACAGGTCAAGAATTGACTG[T/G]T | | | | | |
| AAAGAACATGACATGATAATATAATAT_NODE_8_length_215_ | | CCTAGTTTTGGTATCCAAAATTGTTACAAATATACATAATACA | | | | | |
| cov_33.474419 | | TGAATTTCAGTAGGTTAAACCTGCTAACCATTTAGG | | | | | |
| RAD_kmer_0002417_1_TGCAGGAAAACAACTGCTGGGCGT | 385 | ATATATTGTAGGTTGATTGGGATGAGCCGGAGCTGTTACAAA | G | G | T | G | GYSNP0695 |
| ATGAGGGTTAACTGAAAAGCGTTGTGGAAGGGCGAAACC | | ATGTGAAGCGCGTAAGCCCATGGCTAGTTGAATTAGTG[G/T]C | | | | | |
| ATCTTTTGGTAAATCTGGGGATCGAGGT_NODE_1_length_508_ | | AAACATGCCACTAATTCATCTCTCACCCGTTTCTCCACACAGA | | | | | |
| cov_75.137794 | | AAGAAGCTAAGATTACCTCGATCCCCAGATTTACCA | | | | | |
| RAD_kmer_0002419_1_TGCAGGTAGTTATAAGAAACATGTT | 132 | ATTACATATGTTTTAACTGTGTTATTTTGTTCAAAAAAGGCTA | T | W | T | W | GYSNP0696 |
| AATTGCGTTAGCATACTTTATAGTATTCATTCCTTAGAAG | | TGTGATAGAGCTAATTTAAAAGCTTGGTTTTAAAAAG[T/A]GT | | | | | |
| AAGAATGCAAGTACTAAGAAGGAAAG_NODE_1_length_388_ | | GCGCCTAAGATGCGAGGCGATGGTGGCAACAACCAAAGCAC | | | | | |
| cov_45.644329 | | CTCAGGGTATGGAGGCGGCGCTTCTTGCAATGTAGAT | | | | | |
| RAD_kmer_0002423_1_TGCAGGGGTCAAATTACCAAAGAA | 155 | CTGCAGCAAAATGAAATGACGTCTAAGATACAGGGCAGATC | K | K | T | G | GYSNP0697 |
| GGTAATTCGTCTGGCATATCATGTCAATTTTTTTCGTGCGA | | AGGAAAGGTGGTGGGGGTTAACCTACGGCATTTATACCC[T/G] | | | | | |
| ACTGAGATTTATGGTCTTAGCAGTTA_NODE_1_length_446_ | | TCAGCCCTTCACTAGATTAGTGCATTAGGACATGGGCGTTGG | | | | | |
| cov_161.553818 | | AAGTGTAAGGACCAAGGAGTAAGAGAAGGGTATGATGA | | | | | |
| RAD_kmer_0002425_1_TGCAGGCTACGAGCTGTTCTTTGAG | 300 | CATGAGAGATCGGAACTGGAGCAACTTAAGCAAGATGTTGCT | C | M | M | M | GYSNP0698 |
| GTCTGCTATTTCAGGATTCAATCGTGACAGTAAATCCCTTT | | AATGCTAACAAGCACATACAGAACATCACTAAAGCTCT[C/A] | | | | | |
| CCTCTGGGGTTAAGTGATCAACAAG_NODE_1_length_540_cov_ | | GAGAAGAAGGTTTCGGAGGCTTTCTATTGCCAGCTTGTTGAC | | | | | |
| 213.233337 | | TTATGTCAATCTTCGGATCTAACTGTTTTTATGAATTG | | | | | |
| RAD_kmer_0002425_1_TGCAGGCTACGAGCTGTTCTTTGAG | 356 | CATACAGAACATCACTAAAGCTCTCGAGAAGAAGGTTTCGGA | Y | Y | C | C | GYSNP0699 |
| GTCTGCTATTTCAGGATTCAATCGTGACAGTAAATCCCTTT | | GGCTTTCTATTGCCAGCTTGTTGACTTATGTCAATCTT[C/T]GG | | | | | |
| CCTCTGGGGTTAAGTGATCAACAAG_NODE_1_length_540_cov_ | | ATCTAACTGTTTTTATGAATTGTAAAATTGCAGCTGAAGTTAC | | | | | |
| 213.233337 | | TTGCAAACGTACAGACTCAAATTGATCAGGTCAAA | | | | | |
| RAD_kmer_0002434_1_TGCAGGGTTACATGGGCCACTCTAG | 192 | CACAATAAAGCCCCTAGGGGCTTTATCCACACCCCCAGCCTT | A | A | G | R | GYSNP0700 |
| ATTAGCAAAATAGAAGAAACTATTGATGCAAGTGCCGAT | | AGAATGGCCTAATTTCTCACCCAAAATAAACCTGGTTG[G/A]G | | | | | |
| GGTCCTTTGCTCCTATGTCATACTGGC_NODE_1_length_407_ | | TTTTGTTATCACTAAAAGAACATACTTATACATAACCTTTCTT | | | | | |
| cov_138.012283 | | TTTAACGGCTAACACACACACCTTTTAAAATCTACT | | | | | |
| RAD_kmer_0002441_1_TGCAGGCTGAAATAACTGAATTTGT | 455 | TATGTACTTCAGTAGATAATTAAAAATCTTTAAGTGTATAAG | C | T | Y | T | GYSNP0701 |
| TCATCAACCTCGTCTCCTTGCCAATCCAAACTATTTAAAA | | AGTAAGCTAAAACAATATGGGTTTCTGTTCACAGTTAA[T/C]A | | | | | |
| AAGAAACAATGCACAAATGTTAAAAG_NODE_1_length_651_ | | AATAAACAGGAGGACCGGGATCAAGTTGTAATTCTTTTCCAG | | | | | |
| cov_107.821815 | | GATATGCATGAAGTTGTGACAAGAGACATAATGGAAG | | | | | |
| RAD_kmer_0002446_1_TGCAGGTTCTTAGCATACAACAGCT | 467 | GTGATTATTGTAACAATATTCTTTAGCACCCAAATTTTTCATT | W | W | A | W | GYSNP0702 |
| CTACAGAATCAGTACTATGTATTTTGATGACAAATACGGC | | AAAAATGTTAGAAAGTGAACAAAAGGTGTTTCAGTTC[A/T]A | | | | | |
| ACACATAGTCTTTCTGCAGATGTATA_NODE_1_length_530_ | | CCAACCTGACCCGCTTTTGAACCATTTACCAACCCGCCCATTTT | | | | | |
| cov_55.133961 | | GCCACCTCTAATATCTTGATGCCAGAAATGAGATTT | | | | | |
| RAD_kmer_0002453_1_TGCAGGTTAACGAGGATGTCGAGGT | 110 | CTTTGATACACTGCTGATAGCCCGTTGTCTTTTTCAGAAGAAA | R | G | R | G | GYSNP0703 |
| CAATGCTTGAGTGCCATCGTAATCAATGTCGAGCCATTGG | | GAACGATTTGCTTATCGATCACTTGCAATTCTTTATG[G/A]AT | | | | | |
| AGCTGCTAAACGGTTAGACGCTATTG_NODE_1_length_590_ | | CTTTTGATCTTCCCTTTCCAAATCCTTAGCTTTTCTCTCCATGT | | | | | |
| cov.154.472885 | | TTTTATGCATCGCTAACCTCCTACTCATTTCTTG | | | | | |
| RAD_kmer_0002453_1_TGCAGGTTAACGAGGATGTGGAGGT | 396 | CACCTATTCGGCTAGGAGAAAAGGGAACCGGTCCATCAGCG | Y | T | Y | T | GYSNP0704 |
| CAATGCTTGAGTGCCATCGTAATCAATGTCGAGCCATTGG | | GTTTCTTCAGGTACATACAGAAGACATTTTAGAAGCCAG[T/C] | | | | | |
| AGCTGCTAAACGGTTAGACGCTATTG_NODE_1_length_590_ | | TGTTTAAGGATGTCACAAACCCCTTTTGGGTTCCAAACCAAC | | | | | |
| cov_154.472885 | | ATGAGAACCGCATAATCCAATTTAGAAGTTCACGTTCT | | | | | |
| RAD_kmer_0002458_1_TGCAGGGTGTCGACTTGGGCTGCAG | 212 | TATTAATTTGCGTCGTTTATATATTTCGTATTTTATAGGTGTTC | G | G | R | — | GYSNP0705 |
| GGTGCTCCGCTGGGACAAGGCCTGTGCCCACTTTGATATC | | GCGACGGAGTGGAGCCAAAACGAAGATATTATGGTC[G/A]AG | | | | | |
| TCAATTATTGCTTCTTTGGCTCCAAG_NODE_1_length_382_cov_ | | CGCGAACCGTGGCGAGCATAGAGTATAATAATAATAATAATA | | | | | |
| 106.494766 | | ATAATAATAGTAATGATGAAAAAAAAAAAAAAGAAC | | | | | |
| RAD_kmer_0002461_1_TGCAGGATTTAACATAATCAGCCAT | 375 | TCACCTTTTAAATCCCTCCTTTCAAGTTTTAACAAAACCAACT | Y | T | Y | T | GYSNP0706 |
| TCTATCTTTTCATTGATAATCGGCCTTTTTTTGTATCAAGG | | GTCTGATTATCATGATTACTAACTTGTGTTTAATTAA[T/C]AGA | | | | | |
| ATCAATAATCGATAATCATTAATCT_NODE_1_length_557_cov_ | | CTATAGTTTATATGCTTGCCATCTTCACCAAAGATATTTACTA | | | | | |
| 99.107719 | | TCATGATTTATATCATAAGTTATTTTTCATGTTG | | | | | |
| RAD_kmer_0002472_1_TGCAGGTTTCTTGCCATCTTGAGTG | 373 | TACCAAATAGTGAACATAATAGGAAAGGGGGAGAATAATAT | C | Y | T | Y | GYSNP0707 |
| ACAAAAGGTATGAAGTCGTGTTTGACCGCGTAGGGCAAA | | CGGCTTTTGGAGTCCTGAAAACGGCATCTTTAAGAAGTT[T/C] | | | | | |
| GCACCAACGACCGCATCAAAAACGTCA_NODE_1_length_632_ | | AATAATCGAACAAAGGATCTTAAAGCGGTCACATGGCCCGGT | | | | | |
| cov_135.860764 | | GACACTCATGTTATCCCTAAAGGCTGGGAGATCCCAAC | | | | | |
| RAD_kmer_0002472_1_TGCAGGTTTCTTGCCATCTTGAGTG | 568 | GCAAAAGGCGGGTTTGTTCAGTTCATCGATGCCAACACTGAC | K | T | T | K | GYSNP0708 |
| ACAAAAGGTATGAAGTCGTGTTTGACCGCGTAGGGCAAA | | CCGCGAACCAAACAAGTTATCGTGACGGGTTTCTGTGT[T/G]G | | | | | |
| GCACCAACGACCGCATCAAAAACGTCA_NODE_1_length_632_ | | ACGTTTTTGATGCGGTCGTTGGTGCTTTGCCCTACGCGGTCAA | | | | | |
| cov_135.860764 | | ACACGACTTCATACCTTTTGTCACTCAAGATGGCAA | | | | | |
| RAD_kmer_0002474_1_TGCAGGAGAAGAAATTAAAAGTCA | 139 | TCCCAATTTTACTACTAACTTTTTTGAGGTCTCCAAAATATCA | A | R | G | A | GYSNP0709 |
| AAATGATTATCCTACAGTTGGAAATCAAGAAATATTGATT | | TGGGCCCTTTGAATCAGTGGCCCTATATCCCGTGGCA[G/A]CA | | | | | |
| AGAAGTCATACGTTGTCACGGCCTCCG_NODE_1_length_331_ | | AGGCGTTTATTGATGAAACTGATTAAGATTTGAATGGTCTGA | | | | | |
| cov_16.972809 | | GATTATATGCAGCTAAGAAAGCAAAGCATTTGAGAG | | | | | |
| RAD_kmer_0002476_1_TGCAGGAAATGTGCATATTATGAAA | 531 | AAGTTATTGGCTTTTGATTTTACAGTTTAGGCTAATTGGATCT | W | W | W | W | GYSNP0710 |
| AAGAAAGTAGGAGGCTACATTACTAAAGCTTTAATAAGC | | CATAATAATCTATGATAAGATAGTGCCCAGTTATTTA[A/T]AA | | | | | |
| AAATATTTTCACAACAGACAGTCAATG_NODE_1_length_605_ | | GTTATCTCATTGACTGTCTGTTGTGAAAATATTTGCTTATTAA | | | | | |
| cov_70.803307 | | AGCTTTAGTAATGTAGCCTCCTACTTTCTTTTTCA | | | | | |
| RAD_kmer_0002481_1_TGCAGGTGAAAGATTCAGTGAGATT | 99 | AACATCGGCCAACTCGGCCTTGTACTCGGTCAACCCGGCCTT | G | G | G | A | GYSNP0711 |
| GTGGGTAGTCTATACTACATGGTTCTTGAGGTCCTAGAAA | | GTACTCGACGGTCAAAATCGGTCAACTTCGGCCTACTC[G/A]G | | | | | |
| GAAATTATGGCCCAGAATTTGATGTA_NODE_1_length_522_ | | TCTTGTACTCGGCGAAGTTTGAACTTTAAACATGTATCATTTA | | | | | |
| cov_149.498077 | | AATTATGTTTGTTTTGACATGGATTATGCTTCTTTT | | | | | |
| RAD_kmer_0002484_1_TGCAGGCCATACATATAGAGCTAGA | 132 | AACTTTACTTTTATAAAAAGATTAAGAAGGGTGACCTCA | K | K | T | K | GYSNP0712 |
| GATGGTAATTAGCAGCAGCTGGCCGGAACCGGTGGTGCG | | TGATGGAGGGTTCGTGGGCCATTTAGAATGATCTCTGA[T/G]A | | | | | |
| AGTGCAATCACTCTCAGACAGCGGAAT_NODE_1_length_582_ | | GAAAGAGGGAGGAAGTGAAGGAAGAAGTAATCGCTCCAGTC | | | | | |
| cov_174.324738 | | GAGCTTGGCCCCTTTCTCCACCCCGAGGCGGCTTCCGT | | | | | |
| RAD_kmer_0002487_1_TGCAGGCAGTGGATAACACAACCG | 164 | GTTTGAATAAAGCGCTTTAGGAGATTGTATCCATTAAAAATA | T | T | C | T | GYSNP0713 |
| GATTTTCAAATTTTATGTAATTTTTTCCCTTCCAAAATCTC | | GAGTTTAGATGAGTTTTAACCCATTTGACCCGTTTGAG[C/T]T | | | | | |
| ATTTACCAAGACTCAAAACTTTGTTT_NODE_1_length_586_ | | AAAACACAATCAAAATCGCCCACTATAAGTAAATCAGTCAAA | | | | | |
| cov_106.563141 | | ACTGCCACATCTATGATCTATCCTAACCCGCCCACAC | | | | | |
| RAD_kmer_0002490_1_TGCAGGTTTGGCTTTTGTCCCCTTTT | 199 | ATAACACATACTTTAAGAAAGCATTTGAACTAAGACAAAAAT | A | T | T | C | GYSNP0714 |
| GTCACCTTTGGCCCCTCATGTTTGGTACTTTAGTGACTTGG | | CAAACGAAATAAAGACGAAACCTTAGCATGAAAAAAAA[T/A, | | | | | |
| CAAATTTGGTCCTTCTTTTTTAGT_NODE_1_length_412_cov_ | | C]AAATTCGGAGAAGATAACATCATAGAGAGATAGAGAGAGT | | | | | |
| 66.475731 | | GAAATGTAAAAATCACAAAGGGTACCCGATGGAGATACAA | | | | | |
| RAD_kmer_0002498_1_TGCAGGAGCAAGAACTTCTAGATCT | 216 | TTGTTTTGAGAAGTTTTTGAATTGTGATATGAATTATTGTGGT | A | T | W | T | GYSNP0715 |
| TTAATCGGTTGATACTCCATCCCAAAAACTCGTTGAACAC | | GCTGGTGTGCTTCCGAATAATGTGCATCAAATGCACC[T/A]TG | | | | | |
| CATCAGTCATTGACAGTTTTAGGCAT_NODE_1_length_429_ | | TTGATCTTCCTGGACCCTTTGTGTTGCAGGTATTTTGCGCGTT | | | | | |
| cov_41.794872 | | TTAAGTCTCGGTTTCGTTCTTGTTTGATTATTTTG | | | | | |
| RAD_kmer_0002503_1_TGCAGGTGATATATTAAAAATACAA | 394 | AACAGACGTCCGGTTATCGAAAAACTGACAACTCCGGGATTT | A | T | T | T | GYSNP0716 |
| CCTTTCCCTCTAACTTTACTAACTGACTTATTTAGTTTGTT | | AAACTAACAACTCCTTTGGAGTTTAAAGTTGTGTCACC[T/A]A | | | | | |
| ACTAGACACAACACTATAATCTTCT_NODE_1_length_611_cov_ | | CAGGTGTGATAAACCGAAATATGGGTGGGATTGGACTCGGTA | | | | | |
| 117.21771 | | TAGTTGCAGCACTTGAAGATGTTTCGGCCAAGAAGGT | | | | | |
| RAD_kmer_0002505_1_TGCAGGCTTCAGGTATCTATGCAAC | 123 | GTAATAATAATGACTATTTGTAACATTGACTATGTAAAAGAT | A | A | A | T | GYSNP0717 |
| AAGCCGGATTTGGATCATATCAACTAATGGTTTGACCCGA | | ATATATGAAGTTTGCAGATCAATAAAGAAAATAAAACA[A/T] | | | | | |
| GTCCATGACCGAACTAAGTGGATTGT_NODE_1_length_568_ | | GATCTACCGAACAATTTATGGAACTATGTTATCTAACATGTAT | | | | | |
| cov_173.619720 | | ACATGTGTCTGATGATTGCATAGATTTTCATTTTTTA | | | | | |
| RAD_kmer_0002505_1_TGCAGGCTTCAGGTATCTATGCAAC | 456 | TTGAATGGGTCAGCATTTAGTTTATTTATGTAATGTGCTTTAG | S | C | G | G | GYSNP0718 |
| AAGCCGGATTTGGATCATATCAACTAATGGTTTGACCCGA | | TGTTTGATTGGGTCATGGCCCATCTATATGTGTTAGT[G/C]TG | | | | | |
| GTCCATGACCGAACTAAGTGGATTGT_NODE_1_length_568_ | | ATGATTGGTCAGATAATGATCCATAAGCTAGCTAACGGGTAG | | | | | |
| cov_173.619720 | | ATCACAATCCACTTAGTTCGGTCATGGACTCGGGTC | | | | | |
| RAD_kmer_0002516_1_TGCAGGGAAACCATGAGTGCTGCTT | 399 | TTCAACGAATCTACCACCCCAAAACCGGCTATAATAGTCACC | T | C | T | C | GYSNP0719 |
| TTTTCGGCTATTCTATAGTAAAGTTCTCCTAGAGTTGCGCC | | GCATTAAACGTTTCACATATTCAAGCCTCAATTATATG[T/C]G | | | | | |
| AGTTTGAACCCATGCAGTTTCTTCT_NODE_1_length_595_cov_ | | CCAAAAAACACAAATTATTAATGAAAACCAGAAGCGGTGGT | | | | | |
| 95.826889 | | CATGATTACGAGGGTCTATCATACGTTGCTGACCACCG | | | | | |
| RAD_kmer_0002522_1_TGCAGGTGTGTGATTGGTTAAACTA | 483 | AATGTATCTTTAGCTTCCCTTGTTGCATCAGGATCTTCAAGAG | T | A | A | A | GYSNP0720 |
| TACAATTGTATCCATTTGATAAAGTTAAGATGTTTGCTAA | | GGAACACGTCTATCAGTAAATGCAGTGCATTTTGACG[A/T]AC | | | | | |
| CGTTTCTTTGTAGGTTGCGAACTCGG_NODE_1_length_552_ | | ATCCGAGTTCGCAACCTACAAAGAAACGTTAGCAAACATCTT | | | | | |
| cov_153.382248 | | AACTTTATCAAATGGATACAATTGTATAGTTTAACC | | | | | |
| RAD_kmer_0002523_1_TGCAGGTTCAAATCATGAAACATCA | 331 | CAAGAGGTAATCATGCATATGTGCATTTTAAAACTGTTTTTTA | T | K | T | K | GYSNP0721 |
| TAGGTTAAAATGCGATAATGCAGAAATATTCATAATCTAG | | AATTGGTACTGTTACATTGAAATATTTTTTATTGAAG[T/G]GT | | | | | |
| GAAGCAGACACGCTTAGCAGACGGAT_NODE_1_length_616_ | | ATGATATTTGTATTCCGAATTTGTCTATCTTGACAATATAGTC | | | | | |
| cov_82.144478 | | CTATTTTGTTTCGTTGGTTTCATGTAATTTAACTT | | | | | |
| RAD_kmer_0002523_1_TGCAGGTTCAAATCATGAAACATCA | 385 | TTACATTGAAATATTTTTTATTGAAGTGTATGATATTTGTATT | Y | Y | T | T | GYSNP0722 |
| TAGGTTAAAATGCGATAATGCAGAAATATTCATAATCTAG | | CCGAATTTGTCTATCTTGACAATATAGTCCTATTTTG[T/C]TTC | | | | | |
| GAAGCAGACACGCTTAGCAGACGGAT_NODE_1_length_616_ | | GTTGGTTTCATGTAATTTAACTTCTAAACAGGCGAGGTATCTG | | | | | |
| cov_82.144478 | | GCATCTATTCAGCACCGGAATATTGTGACTCTTT | | | | | |
| RAD_kmer_0002529_1_TGCAGGCGAGAAAAACAACTAGCA | 122 | AGTTAGTTTTGAATGTTAAGTGATCTAATATGAAATTTATTCA | T | T | T | A | GYSNP0723 |
| AGTGCTTCATAAGTTGAGTATTTTTGGAACGTCGGTTGCC | | TTCATTTGTGAAACTATATGATTATTAGTATGTGTTA[T/A]ATG | | | | | |
| GCAATGTCTATGAGCCTGTTTATCAAA_NODE_1_length_623_ | | GAATGCCAAAGTATTGATAGCATTTTTTCATTGAAGTTAAAA | | | | | |
| cov_108.776886 | | AGGGGGCATTTGATGTATAAATTGAAGTCTTTAAT | | | | | |
| RAD_kmer_0002539_1_TGCAGGTCCCAGTGCTTCGATCTAT | 334 | AATTACACCAATGGCACTTCTATAGCTGGTAAATTCTGGATG | C | — | G | S | GYSNP0724 |
| AAACATCGATTTCCGTAACAGCTTGAAACCTAACAGGTTT | | CATAAAAAAAATAGTAAACAGTCATAGTCATACTGAAG[G/C] | | | | | |
| CCCACAGGCTTTTCATCGCAAATAGT_NODE_1_length_672_ | | AAACTCAGCAATATAAGACACCAAAAAACACCCAATGCAAT | | | | | |
| cov_76.909225 | | GATAGACAAAATATCACAACTACATGAAAACATCACTCA | | | | | |
| RAD_kmer_0002539_1_TGCAGGTCCCAGTGCTTCGATCTAT | 405 | ATACTGAAGGAAACTCAGCAATATAAGACACCAAAAAACAC | A | — | A | R | GYSNP0725 |
| AAACATCGATTTCCGTAACAGCTTGAAACCTAACAGGTTT | | CCAATGCAATGATAGACAAAATATCACAACTACATGAAA[A/G] | | | | | |
| CCCACAGGCTTTTCATCGCAAATAGT_NODE_1_length_672_ | | CATCACTCATGAAAACATCACAACTACAAAGATCATTTCATA | | | | | |
| cov_76.909225 | | ACAATCGAAAGCAACTAAACAGAGCTAATTAAATCAGT | | | | | |
| RAD_kmer_0002542_1_TGCAGGGATCAGAAAGTGGGCCTC | 202 | GAGCGTTAACAAAGGAATTTAGGTCGAGACGCGGACGCCCG | — | G | A | G | GYSNP0726 |
| GGTAGGCTCCAGCTGCGTCAGATGGGAGCCCCGTCTGAAC | | GGAGTGAGCATGGAAAATATAATAATATAATATAATATA[A/G] | | | | | |
| TGGGCTCTTGATCTTGTTACTCTTATG_NODE_1_length_413_ | | ACTAATAATAACATTAAAAATAATAAGTAATAGTAGTTATTA | | | | | |
| cov_105.825668 | | TTAAAATAATTGTAATAAATAATAATATATAATGTAAT | | | | | |
| RAD_kmer_0002545_1_TGCAGGTGATTATCTCAACATAAAA | 130 | AAGGTTAGCTATAGTTGGTTTAGGTAGCATTTTTGTACTGGAA | T | T | T | C | GYSNP0727 |
| GACAGGAGCCATCTTTAATAGCAGGTGTTCAGTTGTCATA | | GTTAAGGGCGATTTGTGTCTTCAGATTACATATTGCT[T/C]CCT | | | | | |
| ATATGTTGTTGATCAAACTCTGTAGT_NODE_1_length_524_ | | ATTTGTGAATTTAGATTGTTAAAATATTGCTTTTGATTCAGAA | | | | | |
| cov_102.082062 | | ATTGTGCCGGACTAGCAAATTTATATTTTTTATA | | | | | |
| RAD_kmer_0002547_1_TGCAGGTTTTGAAGAGAGATTCCAG | 149 | AGATTCATCGTAATCTGCCTTCGTTATATGTTGATTTTGAGGA | M | A | M | A | GYSNP0728 |
| AAGGAAGCTAGTCTTTGCTCATCGTCCATTCGTCCTTCTTT | | AACACAACCTTGGCAAGTATGGCACCTCCCACCCATG[A/C]TG | | | | | |
| AGTCAAGGTAATAACTAACACGCGC_NODE_1_length_372_ | | AATACATGGATAAATTCTCTGGCATGTACTCTGGTGCCTGCAT | | | | | |
| cov_48.475807 | | TTCAAAATCCCGCACAATATTATATTAATTTTGTA | | | | | |
| RAD_kmer_0002549_1_TGCAGGAAGCTTTGATGATCTTGTT | 299 | TTTAGAGACTTTCTAAGGGTTTTAGGAGTTTTTTGAAGCATGT | R | G | G | R | GYSNP0729 |
| CTCAATCTCTCTATTGGGGTAATCATATTACACAAATAAT | | TTGACAAATAGGTTAGGTAGCAAACTCATATAAGTTC[G/A]CT | | | | | |
| TTGCATATGTTTTTAGATTGATTTTT_NODE_1_length_409_cov_ | | AAGGTGGCAAGTGAACTCAAGTGAGAAGCAAACTTGCCTAC | | | | | |
| 45.466991 | | GATAGATATTGTTCGCTCGAGTATATATACTGTTATT | | | | | |
| RAD_kmer_0002554_1_TGCAGGATGGGAATGAAGTGTTCTT | 258 | AATCCAATAAAGAAGAATAAATCCAACAAAAAGTTGCATATT | G | A | A | A | GYSNP0730 |
| CAGGATCAAGCGCAGTACCCAGCTTAAGAAGTTAATGAA | | CTGACCCATTCGACCTATCGTTTGAAAAACAAGACCTG[A/G]T | | | | | |
| TGCCTACTGTGATAGGCAGTCTGTGGA_NODE_1_length_450_ | | TGTTTTAAACAATCAAATAAACAAGTGAAATACTTACAGCTG | | | | | |
| cov_126.315552 | | CTTGATAACTAATATATTACCTCTTCAGGAGTCTGCT | | | | | |
| RAD_kmer_0002559_1_TGCAGGGACATCATACCCAAGTCCA | 530 | TTGTGCTGATATAGTTGCATTTGCAGCTAGAGATAGCTTTGAA | T | T | K | K | GYSNP0731 |
| CCTGTCTGCAATAACAACAATTATACACTGTTGAGTCTTG | | ATAGTAAGAAAAATGAAAACTTATAGCCCAAAAGCCT[G/T]G | | | | | |
| GTGCACAATCAGAAAAGCTTGGCCCA_NODE_1_length_637_ | | TCCTAAACAAATATGCAACTTTTGGGCTCAACCAGGCTAACT | | | | | |
| cov_131.142853 | | GGGCCAAGCTTTTCTGATTGTGCACCAAGACTCAACA | | | | | |
| RAD_kmer_0002565_1_TGCAGGGAATCTAAGTCATGATAGC | 511 | TCGTTTGTTGAAATCTTCTAATATTGTTTGTTGGTCTGCCTTTT | C | M | C | M | GYSNP0732 |
| ATTGCTCTCGAATTGTCGATTCCATCTGTGGAATAATTTTT | | TCAGCCCCTGTGATCAAAGCATGAATAATACAAGAT[C/A]TAT | | | | | |
| TTAATTAAAACTATTTATGAACATT_NODE_1_length_610_cov_ | | AGTACTCTGGTTGATATGAAAAACACTTCAAATGTTCATAA | | | | | |
| 133.775406 | | ATAGTTTTAATTAAAAAAATTATTCCACAGATGGA | | | | | |
| RAD_kmer_0002567_1_TGCAGGAAAGTAATTCAGATACTGC | 389 | GGCACCAGGGTCACAAGGATCAGATGGCACAAAGAAAGCAG | G | SS | | G | GYSNP0733 |
| TCAAACTTAAATGACAATTAATAGTGTTGATAAACTAATT | | AGGAGCTAAAAAGGAAAGCTAGAGCAGAGAGGTTGTTTA[C/ | | | | | |
| ACCTGAGTGCCCTTGCTTTCTTTTTC_NODE_1_length_641_cov | | T,G]AGCATAAAACTTTTTACTGCATATTTCAAATTTAATTTCC | | | | | |
| 118.243370 | | AGACTCTATACTCATCACTTTTTATTGTATTTCTGTGTTG | | | | | |
| RAD_kmer_0002567_1_TGCAGGAAAGTAATTCAGATACTGC | 501 | TTAATTTCCAGACTCTATACTCATCACTTTTTATTGTATTTCTG | Y | C | C | Y | GYSNP0734 |
| TCAAACTTAAATGACAATTAATAGTGTTGATAAACTAATT | | TGTTGATCAGATTTGGTATCACTAAGTCAACACCTA[C/T]AGA | | | | | |
| ACCTGAGTGCCCTTGCTTTCTTTTTC_NODE_1_length_641_cov_ | | GGAAGAGGAGAAAAAGAAGGCTAGACTCTCCAGGTTTGGAT | | | | | |
| 118.243370 | | CACCTTCTAGTGCTGATCCTAAAGAAGAAGAGAAAA | | | | | |
| RAD_kmer_0002568_1_TGCAGGAAGAAAACACAGCACCAA | 113 | TAAAAAATAATAAATTTGAAGTTAAAAAGGGAGCCTACAATT | R | A | S | — | GYSNP0735 |
| CACCAAAGCAACTCCAGCGACGACACTATAACCCAAATG | | GTCAAGCCTCTTTTGACAAAAGCAACTGTGAAACCGCT[T/A,C, | | | | | |
| TTGAACGAATGAAACAAGATGAAGGCTA_NODE_1_length_ | | G]GTGATTTTATGAAACAATTAAATTGCTCTTTAACAAAAACC | | | | | |
| 634_cov_111.077286 | | ACCATGGTTTTGTAAAAACCATTATAAAACCCATTTATG | | | | | |
| RAD_kmer_0002568_1_TGCAGGAAGAAAACACAGCACCAA | 505 | CGATCGGAATCGACAGAATGCAAGTTGGTGATTGTTAGGGTT | C | T | C | C | GYSNP0736 |
| CACCAAAGCAACTCCAGCGACGACACTATAACCCAAATG | | CTACTGTAGGTTCTCACGTTGGCATAGGCATGGATCAA[C/T]G | | | | | |
| TTGAACGAATGAAACAAGATGAAGGCTA_NODE_1_length_ | | AAATGTTGTCAGCGTGAAACTAATTGCCGGCGCCGCCGCCAT | | | | | |
| 634_cov_111.077286 | | CTCGGCTGCAAACCACCATTGTAGCCTTCATCTTGTT | | | | | |
| RAD_kmer_0002572_1_GCAGGACTTCACGAGGGATGTGCA | 462 | AACCGGTTTCCGTTGCTCAAGACTGCGTATGATGGGGAGAAG | Y | T | Y | C | GYSNP0737 |
| AAGCATTCCCATTCAAAAAATCTTGTAGCTTCGAAAGGTT | | AATATTTTCAGTGCGGTATTATTGGAACCGGGAATTTT[C/T]A | | | | | |
| TAGCTCGTTTCCAAACTTGATGGTGC_NODE_1_length_558_ | | ATGTTCAATTATATGGTCGGTCTTATGTGTGCACCATCAAGTT | | | | | |
| cov_156.786743 | | TGGAAACGAGCTAAACCTTTCGAAGCTACAAGATTT | | | | | |
| RAD_kmer_0002573_1_TGCAGGTTTGGCTTTTGTCTCCTTTT | 92 | TCCATACCAGAAACTTATCAAATGCTAAAGCTCATCAAATGA | A | W | W | W | GYSNP0738 |
| GTCACCTTTGGCCCCTCATGTTTGGTACTTAAGTGACTTGG | | AAATCAGCAAACTAACACATGTTTTTCAAAACCCCAAA[A/T]A | | | | | |
| CAAATTTGGTCCTTCTTTTTTAGT_NODE_1_length_233_cov_ | | GCCCTAAACATAAAATACTTATGGAAATTAGATCTTCAAAGG | | | | | |
| 11.334764 | | AAGCCATAATTGAAAAGCAAATCTAAAATCAACAGAA | | | | | |
| RAD_kmer_0002575_1_TGCAGGATGACTATTTGATATATTG | 396 | TCTAACAATATATCAAGAACCCAAACTAGACGCATCTCACAT | W | W | T | T | GYSNP0739 |
| CTTGAATTCAAAGTAGATGTGAGATGCGTCTACTTTGACT | | CTACTTTGAACTCAAACAAAATATCAATGAACCCAAAC[T/A]A | | | | | |
| TTTGGTATTTTGTTTGAATTCAAGG_NODE_1_length_479_cov_ | | GATGCATCTCACATCTACCTTGAATTCAAACAAAATACCAAA | | | | | |
| 69.929016 | | AAGTCAAAGTAGACGCATCTCACATCTACTTTGAATT | | | | | |
| RAD_kmer_0002587_1_TGCAGGGACGACGAGATCAACCCC | 482 | AGTGTAGATCAGACCTCACAGGTCACTGGCCAGATGTTTGCA | C | C | T | — | GYSNP0740 |
| ATCACAATGATGTTACTAGATCAAAAAGACGACGATGATT | | TCTGCTGCGATTCTTGAACCACTAGAAACCACAACAGA[T/C]G | | | | | |
| CTCAACCTAACAAGGTTAGCTTGACAT_NODE_1_length_562_ | | GTGATGGTGGTTCCATGTCAAGCTAACCTTGTTAGGTTGAGA | | | | | |
| cov_90.197510 | | ATCATCGTCGTCTTTTTGATCTAGTAACATCATTGTG | | | | | |
| RAD_kmer_0002589_1_TGCAGGGCTTATTTGATAATAAGGA | 445 | GGAAAACTGTCTTTCGGGGAGGCTTTATCTAGGTATCTTTTCT | A | A | C | M | GYSNP0741 |
| GCTTCAGGCAACTGCCGCTTCACCTGCAGACCCCAAACAG | | ATAGCAGTTACTTCTCTCGTAATCTCGATACTATAAA[C/A]TG | | | | | |
| GTCTCACCCAAGGGTCTTCCTTTCCA_NODE_1_length_581_ | | CCAGTAATTTTATTTGTGTATTTGTCATTTTAGATGTAAAGCT | | | | | |
| cov_164.000000 | | CAAGATGTACCCATATGCCTCATGTATGGAAAGGA | | | | | |
| RAD_kmer_0002595_1_TGCAGGGATGTAACCTTAGATGCCT | 395 | TTTTCTCAAATTTATAAATTTAATGCATACCTTATAGTAAAAC | T | T | T | A | GYSNP0742 |
| CGTCCATTCTTCGATACTACAAGCGTACACTAGCTGCAAT | | TGTCTTTAATTATGGAACGATATGATTGTATATAACA[T/A]AA | | | | | |
| AGCACCACAAAATGCTAAATGTATAG_NODE_1_length_594_ | | TCTTGGGTTGCGGTGATATACTGCATATTCTTATAAGTACTTG | | | | | |
| cov_139.710434 | | AATAATTATCCCTAAAACTAGAAGCATTTAAATTT | | | | | |
| RAD_kmer_0002606_1_TGCAGGCAAAGCAATCCAAGAATG | 402 | TGAATTTATACGCACGTATAAAGTAGTTTGTGTTCTGCCAAA | A | T | A | — | GYSNP0743 |
| CCAACACGTGAAGCCGATTTATACCGGCCAACTTGTCCCT | | AGTGCATGTATATTATTGAATAACAAGAGTCTTTAAAC[A/T]G | | | | | |
| CCAGGTTGTGCAAAGTCTTTTGAAGTG_NODE_1_length_632_ | | GCAAACAAACAACTTTAAAACTAAACAAACCAATACACAAA | | | | | |
| cov_114.452530 | | AGACCGCATGTTTATTTTGCAACAAACTATCCTAAAAC | | | | | |
| RAD_kmer_0002606_1_TGCAGGCAAAGCAATCCAAGAATG | 472 | GTCTTTAAACAGGCAAACAAACAACTTTAAAACTAAACAAAC | T | C | T | — | GYSNP0744 |
| CCAACACGTGAAGCCGATTTATACCGGCCAACTTGTCCCT | | CAATACACAAAAGACCGCATGTTTATTTTGCAACAAAC[T/C]A | | | | | |
| CCAGGTTGTGCAAAGTCTTTTGAAGTG_NODE_1_length_632_ | | TCCTAAAACCTGGATTCCTCATGACCCATTCTTATTCTAACTT | | | | | |
| cov_114.452530 | | AACGATTGCTGCAAACGGTCACTTCAAAGAGCTACC | | | | | |
| RAD_kmer_0002616_1_TGCAGGTTTTGGTTTCCAGATATAA | 539 | TGTTAAAATAATACAACTTCTCAATGGTATTTGACAAACTAA | A | A | T | A | GYSNP0745 |
| GAACTAGCTAAGAAAATGTGCACTTTGACTTTTTTAAGTC | | CTCTTAAATTGTTTTACAAAAAAGTTTTGTGTCACCTA[T/A]CA | | | | | |
| AAACTGACTGATATGAAACTTGATAG_NODE_1_length_600_ | | AGTTTCATATCAGTCAGTTTGACTTAAAAAAGTCAAAGTGCA | | | | | |
| cov_124.279999 | | CATTTTCTTAGCTAGTTCTTATATCTGGAAACCAAA | | | | | |
| RAD_kmer_0002619_1_TGCAGGTATCGCACACACCAGGGCA | 239 | ACTTTCATCCTCATCTCAACCATCTACTTTCTTCTAGTCAATC | T | T | C | T | GYSNP0746 |
| GAAAACAATCTTGTAATCATTATCAAACTTCTCAAACTTG | | TCCCGTCCTTGACACTAACCAAATGCCGCTAGTAAC[C/T]GGC | | | | | |
| AACCAATTGCTTAAGGTCTCTTTGCC_NODE_1_length_604_ | | ATCTCGTACTACATCGTGCCCGCCGTTAGCGGCAGGGGTGGC | | | | | |
| cov_139.549667 | | GGTGTAAAACTATCCCCGACAACGCTAAACCAGAC | | | | | |
| RAD_kmer_0002619_1_TGCAGGTATCGCACACACCAGGGCA | 309 | CGCTAGTAACCGGCATCTCGTACTACATCGTGCCCGCCGTTA | G | G | C | G | GYSNP0747 |
| GAAAACAATCTTGTAATCATTATCAAACTTCTCAAACTTG | | GCGGCAGGGGTGGCGGTGTAAAACTATCCCCGACAACG[C/G] | | | | | |
| AACCAATTGCTTAAGGTCTCTTTGCC_NODE_1_length_604_ | | TAAACCAGACATGCCCACTCAACGTGGCGCAAGAAAACAAC | | | | | |
| cov_139.549667 | | GAACAAAGAAATGGGCAACCATTGGATTTCCAACTAGCC | | | | | |
| RAD_kmer_0002619_1_TGCAGGTATCGCACACACCAGGGCA | 425 | AACAACGAACAAAGAAATGGGCAACCATTGGATTTCCAACT | T | T | G | T | GYSNP0748 |
| GAAAACAATCTTGTAATCATTATCAAACTTCTCAAACTTG | | AGCCATCCCCAACAGAGGCGGTGTCATTCGTGATAACGC[G/T] | | | | | |
| AACCAATTGCTTAAGGTCTCTTTGCC_NODE_1_length_604_ | | GATCTTAACATTAGATTCACGGGTGCGACAACATGTGGCAAG | | | | | |
| cov_139.549667 | | CCTGCTGTTTGGCGGGTCGAAGAGGTCAACGGGCAACG | | | | | |
| RAD_kmer_0002622_1_TGCAGGCTGCAAGGCGGGAAATAT | 480 | CAGATGAGCCCGTCTTTGACCATGGCTTCACATTTAATGTAG | R | — | R | G | GYSNP0749 |
| AAATTGCTCCAGTTTACTTGAAACAAACGATCCCGATCAT | | GTTTGGTCACCACCATTTGCTGAATCGACTGTTTTTGA[A/G]C | | | | | |
| TCAAACATAATGGCCTGTGATATTTGC_NODE_1_length_589_ | | AAAGTATACAACAACAATCGTGACAGAAACGAGGCTCGATG | | | | | |
| cov_129.030563 | | CAGCAAATATCACAGGCCATTATGTTTGAATGATCGGG | | | | | |
| RAD_kmer_0002625_1_TGCAGGGTTGATGGTATCAAATTTA | 453 | ACCTTACATAAACTGCAGCAGACAAAAGTTAGCATCTGTAGC | T | T | T | C | GYSNP0750 |
| AACTGTTGGTCTTTTTCTTTGTTCGGTTTTTTACCATGTGTG | | ACATAGCCCTGTAGCATGAATAACTCCTCAAATAATAA[T/C]A | | | | | |
| TGTTATGCTAGCATCATCTTTTTT_NODE_1_length_523_cov_ | | ATTAAAAAAAGATGATGCTAGCATAACACACACATGGTAAA | | | | | |
| 138.927338 | | AAACCGAACAAAGAAAAAGACCAACAGTTTAAATTTGA | | | | | |
| RAD_kmer_0002626_1_TGCAGGATTTCAGAGGTTCGGAGGG | 237 | TGGCGCAAAAACACAAAGACAATCACCAATCGAGTCCCCCTA | A | G | A | A | GYSNP0751 |
| GGTAAATCTCATGTGATTAGTCGTCTTTTTATATAATGAAT | | AAGTGTAAGAAGCTCTAAGTCTCTGAGACAAAACTAAT[A/G] | | | | | |
| ATAGTATAAGTTTTTTCCTTGATTT_NODE_1_length_571_cov_ | | CTCTAATGTACCCTAAAAGTCCTTGACCTCAATCCTACGCCTC | | | | | |
| 77.809105 | | CACGTAGTCCTATTCTGGAAAATGTCCTCAGAGAGGT | | | | | |
| RAD_kmer_0002628_1_TGCAGGTTTACAATGGCCACACCAA | 337 | AAGTCTCTATTGTTTATTTTTGATTTCATACGCTAACGCTAGA | — | T | A | W | GYSNP0752 |
| GGAGCATAGAACTAAAACAGCTTTAAAATTTGCAGAGCTT | | GAAGTTATGCTTGATTTCATAAAAATCATAGGGATTT[A/T]AA | | | | | |
| TATTATAATTCTGATTTTGGGGATCA_NODE_1_length_471_ | | GCTCGAGACTTTCTTGAAACTGTCTTCTTTATGTTATACAGT | | | | | |
| cov_96.490448 | | TTCAAGGACTAGCAAGTGTTTCTATGATCCCCAAA | | | | | |
| RAD_kmer_0002631_1_TGCAGGTGCCCTTTGCCGACAACTG | 147 | TATGTACCTATATAAAACAGTTAGTTTATGTAGTGGTGAGTAT | K | T | K | T | GYSNP0753 |
| CAACCACAGAACCATGTTCGCGTGCTACTTTCAGCAAGCT | | ATTTAGAGACTATTGACACAACATGTTTCATAAATAT[T/G]AT | | | | | |
| GGAAGACATGTACCTACAAAACCATG_NODE_1_length_610_ | | GGACCTTTTCTTTAACTTTATATATACCACAAGCTTGCACTTG | | | | | |
| cov_161.704926 | | ATTGTTGTAATTTATTCTAAAACTTAATCAAGGTT | | | | | |
| RAD_kmer_0002633_1_TGCAGGAAATGGCTCCTGCAAAGCA | 234 | TACATAAGTTATCACAGCTAATTATATGATTTTGCTTATTCAA | R | G | G | A | GYSNP0754 |
| AATGAAAGTTATGCAAGAATTTCAGAAGCAATCTGCACA | | TATCATTTAATCACTTTCAATATAAATATCCAAAAAC[G/A]CC | | | | | |
| AATGGATATGACAGTAAGATACAGCAC_NODE_1_length_609_ | | CATTATCATTCACGCTACAAAACAGCTTTATTAAGATTACCTG | | | | | |
| cov_140.876846 | | GTTTGTTAGCTCATCGGTTTCATCTTCAGCTTCAT | | | | | |
| RAD_kmer_0002652_1_TGCAGGCGAGGGCGGGTCTCCCGA | 277 | TGATGAAGAGGCTGATACATGGAAATAAATGAGTATTGGATT | C | — | A | C | GYSNP0755 |
| AACTAAAAGAGAATAGGGACCATGACAACTTGCAACAAA | | AGAGTCGATTGTGGATTAAAGTCCTTAATCGGTTCGGC[A/C]A | | | | | |
| AGTTCTAAACAAACAAAAATAAGTAACA_NODE_1_length_ | | CTGAATTGACTAGCAACAGGTTTGTAGCTCAATCTGTGGAGC | | | | | |
| 462_cov_26.071428 | | GTATACTCTTCCCACTATTTGAATGGTGTCTGGTACC | | | | | |
| RAD_kmer_0002664_1_TGCAGGCTCAGCTGTTTATAGCCTT | 161 | TTGAAGGTAAGGAACGAAATGATTCTTGGGGAGATGAAGCC | A | A | R | — | GYSNP0756 |
| TTTGTTTTATAAGTTATTAGTGGTGTTGCTTGTTTAAACTT | | ACTATGTTTTCTTTGGTTTTAAATATAGATCTCTTAGTC[G/A]G | | | | | |
| TTAAAAAATAAATGTATAGTAGAAT_NODE_1_length_570_cov_ | | GGAGAGTTGTGTAAATTTTCCTTGTATGTGTTGTAGCCGGTGT | | | | | |
| 120.042107 | | TGTGTCTTACGTCCCCTTCCAGTCCGGTAGGGTGGC | | | | | |
| RAD_kmer_0002664_1_TGCAGGCTCAGCTGTTTATAGCCTT | 214 | TTGGTTTTAAATATAGATCTCTTAGTCGGGGAGAGTTGTGTAA | T | T | W | — | GYSNP0757 |
| TTTGTTTTATAAGTTATTAGTGGTGTTGCTTGTTTAAACTT | | ATTTTCCTTGTATGTGTTGTAGCCGGTGTTGTGTCTT[A/T]CGT | | | | | |
| TTAAAAAATAAATGTATAGTAGAAT_NODE_1_length_570_cov_ | | CCCCTTCCAGTCCGGTAGGGTGGCATTCTTTGTTAATGAAGTT | | | | | |
| 120.042107 | | AACAGTTCAAAAAAAGGGGGACATGGACCTGGTT | | | | | |
| RAD_kmer_0002664_1_TGCAGGCTCAGCTGTTTATAGCCTT | 333 | ATGAAGTTAACAGTTCAAAAAAAGGGGGACATGGACCTGGT | T | G | G | — | GYSNP0758 |
| TTTGTTTTATAGTTATTAGTGGTGTTGCTTGTTTAAACTT | | TTGTTACAGAAAACCCTAATTTTCTTCATGTAATGTTAA[G/T]T | | | | | |
| TTAAAAAATAAATGTATAGTAGAAT_NODE_1_length_570_cov_ | | TGTTTAAGCTGGCCATTCAAGGGCATAACAAGGCCAAACTTG | | | | | |
| 120.042107 | | CGAGAGTTATTAGATACGTGGTTGTATAATTGTCTT | | | | | |
| RAD_kmer_0002665_1_TGCAGGTAGCTTAACTTACCACATG | 288 | TAAATGCATGACTGACATGTGCTATGCAGGAGTTACCTCATT | W | W | A | W | GYSNP0759 |
| CATGCATTTGCAGGGAAAACATACAGATTCAGAATATCA | | GATCGGGCCTGCATGAGGGACCAGGCAATCTGGGTGGT[A/T] | | | | | |
| AATGTGGGGTTGACAACATCCATCAAC_NODE_1_length_601_ | | GGGCCACCAGGGGGAGGACCGGAGACCCTGATTCTGGAGTT | | | | | |
| cov_127.241264 | | GCGGTAGTGGAGAATAGCAGTGGTCATGAGCACGCGACG | | | | | |
| RAD_kmer_0002670_1_TGCAGGTTTGGCTCATGTTGATGAA | 294 | GTTTCTTTGCATACTAGTTTTTTTAGAAAAAAGGATGGGGTTC | T | — | Y | Y | GYSNP0760 |
| AATAGCTTGGTTTAGTTTCTTTTTACTCATTCAAAGTCAAA | | TATGCATAGTCTTATGAAAGCATATGACAGTAGTAAG[C/T]TA | | | | | |
| AGTAAAGAAATTTAAAAAGTTTTTCA_NODE_1_length_361_cov_ | | TGAAAACTTTTTAAATTTCTTTACTTTTGACTTTGAATGAGTA | | | | | |
| 112.612190 | | AAAAGAAACTAAACCAAGCTATTTTCATCAACATG | | | | | |
| RAD_kmer_0002674_1_TGCAGGTTTTGTCTTGGTTCCCTGTG | 159 | ATAACTTTTAGTGTACATTTTAAATATTTATATAAATGGGTGA | T | Y | Y | Y | GYSNP0761 |
| GCGAAAGTTTGACCATCGGGATGCCATGGTGATGCAAATG | | ATTACATCAATATGTATCCAAGTATCCAATTATACAC[C/T]TG | | | | | |
| AGAAATCCCAGTGTCCATTTAAAGA_NODE_1_length_627_cov_ | | TTTGTTATTGATGTATTAAACTACCAAATGTACCTTTTGATGT | | | | | |
| 109.550240 | | ACCAAATTTCCAGTTCTTGCTATTGTATGTATTTG | | | | | |
| RAD_kmer_0002674_1_TGCAGGTTTTGTCTTGGTTCCCTGTG | 217 | ATCCAAGTATCCAATTATACACCTGTTTGTTATTGATGTATTA | A | R | R | R | GYSNP0762 |
| GCGAAAGTTTGACCATCGGGATGCCATGCTGATGCAAATG | | AACTACCAAATGTACCTTTTGATGTACCAAATTTCCA[G/A]TT | | | | | |
| AGAAATCCCAGTGTCCATTTAAAGA_NODE_1_length_627_cov_ | | CTTGCTATTGTATGTATTTGACCTTTTAGAACTTGTAAAACGG | | | | | |
| 109.550240 | | TGATGTGGCAACTTATATGGCTGCCGCGTCACCGG | | | | | |
| RAD_kmer_0002674_1_TGCAGGTTTTGTCTTGGTTCCCTGTG | 450 | TATAATTGAATACATTTCCATGTAGTTTGCCCTGTACATTCCC | G | R | G | R | GYSNP0763 |
| GCGAAAAGTTTGACCATCGGGATGCCATGCTGATGCAAATG | | GTTTTCCCAAAACAAATGGGAACTGGTTTGCCCTATC[G/A]GT | | | | | |
| AGAAATCCCAGTGTCCATTTAAAGA_NODE_1_length_627_cov_ | | TGTGATTTTTTCAAGCTTAGCGGTAATTGTCTCACTTACATT | | | | | |
| 109.550240 | | ACACAGTTAAGAGTTATGAAAACTTTTACGACAAT | | | | | |
| RAD_kmer_0002676_1_TGCAGGATATAAGCAAGAAAGAAT | 168 | TCAAACATAAAAAGTGATTATGTGATATTGAGTAATGGAAAT | T | W | W | T | GYSNP0764 |
| AGCGGGTATGGGTCTTAATGAAGAAGAACTCAAGGCGAA | | CAGATAATTAGCTGTAACAAAATGGGTTATTAGAGATA[T/A]T | | | | | |
| TAAAGTAAGAATCAATATACCCATGTGA_NODE_1_length_ | | GTTGGATGTGCCTTGGTTGTTTGAAGTTAACTATTAATTGAAA | | | | | |
| 634_cov_110.190849 | | TTACAATAAGTAGAATATCAAACATAGTTATTACCT | | | | | |
| RAD_kmer_0002676_1_TGCAGGATATAAGCAAGAAAGAAT | 308 | TATCAAACATAGTTATTACCTCATTATAGCCTTGCCACCTGGC | R | G | R | G | GYSNP0765 |
| AGCGGGTATGGGTCTTAATGAAGAAGAACTCAAGGCGAA | | TTAAGGACTCGACGCATCTCCTTGAATACTTCAATCG[G/A]TT | | | | | |
| TAAAGTAAGAATCAATATACCCATGTGA_NODE_1_length_ | | TTGTCAAATAGTCAACACTAACCTGCCGTGCAATACGGTATT | | | | | |
| 634_cov_110.190849 | | TAGTAATAGCCCGGTTCAAGAATATGTGAATAACTA | | | | | |
| RAD_kmer_0002676_1_TGCAGGATATAAGCAAGAAAGAAT | 567 | GACCTACAGCGGTTATAAATGTAACAATGATAAAATCAATTA | R | G | R | G | GYSNP0766 |
| AGCGGGTATGGGTCTTAATGAAGAAGAACTCAAGGGGAA | | ATGTAAAAAATCTTTCACATGGGTATATTTTTTTGAAC[G/A]G | | | | | |
| TAAAGTAAGAATCAATATACCCATGTGA_NODE_1_length_ | | CTCACATGGGTATATTGATTCTTACTTTATTCGCCTTGAGTTC | | | | | |
| 634_cov_110.190849 | | TTCTTCATTAAGACCCATACCCGCTATTCTTTCTTG | | | | | |
| RAD_kmer_0002688_1_TGCAGGCAGGATAATATGATAGTTG | 375 | AGTTGTAATTTGTAAACTATCCCTAACATACAATGATAAGTA | G | R | R | G | GYSNP0767 |
| ATTTTTATTAATTAGTGTGTATAAGTTAGCTGTGTGTTATC | | AACTATAGTACCTATAGATAAAAGGAGCAAGTAGACAA[G/A] | | | | | |
| TGATTTTAGTACTGCAAGCATAATT_NODE_3_length_524_cov_ | | AAAACAAGCACTAACCACTGTCTAGGAAATTCTGCTTAACC | | | | | |
| 38.881680 | | AAATCAAACCAATTACACTCACTAATTAGTAAAAAACC | | | | | |
| RAD_kmer_0002694_1_TGCAGGCTCTTTGAAGGTAAAACTT | 480 | AGTGCATGTGGTCTTAATTCACTGCACAGTCTCATAACCTCTT | R | R | G | R | GYSNP0768 |
| GTATAAAAAGATGGAAGTATAAACTTTATAAAATAAAAA | | TTCTAACCACTGCAGCATTATCTGTTGACAAGTAACC[G/A]TA | | | | | |
| CTAAATATTGCTATGATTTTTGTTGCA_NODE_1_length_617_ | | TCGAAGAAACGATATGTCTTCTTCCATCACAATTGTTGTGTAC | | | | | |
| cov_110.307945 | | ATTGATCTCATTAAACCTAACACGTCCTGCAACAA | | | | | |
| RAD_kmer_0002697_1_TGCAGGTACAACATATAATTAAAAT | 314 | GTACGCAAAAATATTTTTTACAATTAAAGATGTTAGAGTAAT | T | Y | C | Y | GYSNP0769 |
| CAAAATGTAGCATACTAGCATATATGTGTGAAATACAATA | | CACTTGGTCAAATATTTACAGAAAGAGGATTCATGTAA[C/T]G | | | | | |
| AAGTTGTATATCATGACATACACATT_NODE_1_length_494_ | | CCTGTGGTGAATCTGGGAATCTGCTTGTTTGTGAAACGTGCA | | | | | |
| cov_93.997978 | | CTTATGAGTACCACCCTAAGTGTTTATTACCACCTCT | | | | | |
| RAD_kmer_0002703_1_TGCAGGTGGTGGTGGTGGCGGTAGA | 205 | GATCTGGTTTCATATGTATAGACTTGCATGTTTGTGTATGCAT | A | M | M | M | GYSNP0770 |
| GATGAGCACATAAGAGTGTTTGGAGTCGTTGAACCACCAC | | AAATTTACAGAAAGGATAGCTTCATAACACATAGAGC[A/C]T | | | | | |
| CACCAAAGGTGTACATTGAGTGTTCT_NODE_1_length_636_ | | TCTGTGATGCTCTAATGGAAGAAAACTCAAGAATTGCATCAC | | | | | |
| cov_159.762573 | | GTCCACTAGTTCCCAACATGACCTTTCGGAATGACAT | | | | | |
| RAD_kmer_0002703_1_TGCAGGTGGTGGTGGTGGCGGTAGA | 544 | CCATCAAGCTCAAATTACAACAATGTTGGAATGTTACAACCG | R | R | R | R | GYSNP0771 |
| GATGAGCACATAAGAGTGTTTGGAGTCGTTGAACCACCAC | | GATATGATGAATTGGTTGGGTAGATACAATAGTTTACC[A/G]C | | | | | |
| CACCAAAGGTGTACATTGAGTGTTCT_NODE_1_length_636_ | | AAGGGTTATCATTAAAAGATGAAGAACACTCAATGTACACCT | | | | | |
| cov_159.762573 | | TTGGTGGTGGTGGTTCAACGACTCCAAACACTCTTAT | | | | | |
| RAD_kmer_0002704_1_TGCAGGGTTGGAACTTTTGCTGTGT | 185 | ACGGTCGTGGCATTAGTAGTGGTTTAACATGATAATGATGGA | A | C | C | C | GYSNP0772 |
| CATGGCTGTTGGGAACGTTCTTGATGGTGTCGGTTATCCC | | GGTTTAGCGGTGAACTAGAATGAACTCGCCACTTTCTT[C/A]T | | | | | |
| ACCAATCCTGAAAGGGCCGAGGAGTT_NODE_1_length_653_ | | TTTTGAACAGCAGTGAGATTTTACAACTGACCAAAGACGCAA | | | | | |
| cov_133.150070 | | GTAGCTAGACAAGTAGACAGTACAAAAAAGATCACCA | | | | | |
| RAD_kmer_0002708_1_TGCAGGCATATGCCACGAAGTTATA | 233 | ATGCTAAAGAAGCTGTTGAAATGTTTGACCAAATGACAACGA | A | G | G | G | GYSNP0773 |
| TCCAGCTACATCCTGCTTTTTTCTTAATTCCTTTACCACAC | | ACAAAGTCAAAGTCAAACCCGACTTGACCACATTTCTT[G/A]T | | | | | |
| ATGAGATTATGTACTCCAGCAGAAT_NODE_1_length_646_cov_ | | AGTAATTTCTGCTTGTAGTCATGTAGGATGGCTAGATAAGGG | | | | | |
| 117.075851 | | GATAGAATATTTTGAGTTGATGAAAAACGATAGTTCC | | | | | |
| RAD_kmer_0002708_1_TGCAGGCATATGCCACGAAGTTATA | 309 | TCTTGTAGTAATTTCTGCTTGTAGTCATGTAGGATGGCTAGAT | A | G | G | G | GYSNP0774 |
| TCCAGCTACATCCTGCTTTTTTCTTAATTCCTTTACCACAC | | AAGGGGATAGAATATTTTGAGTTGATGAAAAACGATA[G/A]T | | | | | |
| ATGAGATTATGTACTCCAGCAGAAT_NODE_1_length_646_cov_ | | TCCCTTGAACCATTGAAAGCCGAACATTATGCTTGTATCGTG | | | | | |
| 117.075851 | | GATCTTTATGCTAGAGCTGGCTATTTAAATGAAGCAG | | | | | |
| RAD_kmer_0002708_1_TGCAGGCATATGCCACGAAGTTATA | 410 | CCGAACATTATGCTTGTATCGTGGATCTTTATGCTAGAGCTGG | — | C | Y | C | GYSNP0775 |
| TCCAGCTACATCCTGCTTTTTTCTTAATTCCTTTACCACAC | | CTATTTAAATGAAGCAGAAGAATTTATTAAAAGCATG[T/C]CA | | | | | |
| ATGAGATTATGTACTCCAGCAGAAT_NODE_1_length_646_cov_ | | ATAGAAGGCGGACCCGAAGTTTATAGAGCACTGTTAAGTGGT | | | | | |
| 117.075851 | | TGTAGACTTCATGAAAATAAGGAAACTGGTGTCCGT | | | | | |
| RAD_kmer_0002709_1_TGCAGGAGATTCTTTACAGCTTAGC | 147 | AGACGTAAAGGACTGAGTAAGCTAGCTGCACATTCTTATTAT | T | A | T | W | GYSNP0776 |
| ATCTCAAATACTTCTAACGAATAATCGCGATCGATTGTAA | | AAATTATTAATAAATATATATTTAACTTGGCACATAGT[T/A]A | | | | | |
| TATAAGAATATATGCTTCTGCTGAAA_NODE_1_length_559_ | | AAAAAAAAAAACCATGTTTGAGTACTGTGCTTTGACTAAGAG | | | | | |
| cov_77.135956 | | GGTAACTTGTGCTTTGACTTTTGACACTGATCAATAC | | | | | |
| RAD_kmer_0002719_1_TGCAGGGAAGAAAAACTAGTGGAG | 134 | AAGCTTCCATGCCCTAGCTAAATTCAGTTGATTAATCATGTGA | C | Y | Y | T | GYSNP0777 |
| CGCGCAAAAGCACTTAAAGTAAACATTGGAACTGACCCT | | CAAATAGTTCAGTTTGTTTTTTTAAGCATAAGGGTAA[C/T]AT | | | | | |
| GATACAGACATTGGCCCTGTGATCAGTA_NODE_3_length_419_ | | GGTCATTTCACATGGATTTAACTTTTAACGTCTTGAGTGAAAA | | | | | |
| 19_cov_38.470165 | | GGACCAAACACACACGTGTTACTGTTTGGAAAACA | | | | | |
| RAD_kmer_0002726_1_TGCAGGTTAACCAAACTGAATATGG | 346 | AATAATATAGATAAAGATACCTAATAATATTATTATATATTA | W | A | A | — | GYSNP0778 |
| TCTACCGTTTTCTTGAATCTATACAATTATATATTTCAATA | | TTAACTTAATAATATTTTTGAAATTAAAAAATTGAAGG[A/T]G | | | | | |
| TAATACTCGCACCATATTGATTCTC_NODE_1_length_454_cov_ | | ATAATATTGATGACGTGACTTATTGTGGTGTGAGAATGGTAT | | | | | |
| 95.429512 | | GAGAATCAATATGCTGCGAGTATTATATTGAAATATA | | | | | |
| RAD_kmer_0002728_1_TGCAGGTCTACAATATTCTCATCTTC | 386 | TTTGAGGACAAACATTTTTTTTACGAATGGGAGAATAATAGA | G | A | R | R | GYSNP0779 |
| TACGAAAACGTTTGACCTCACACTTGGAAAAACATTTATG | | GCTCAGGGTGATCCGTTACCCATCGCCTCAATCCGACA[A/G]A | | | | | |
| AATTTACTTGAATTATAATCATACC_NODE_1_length_537_cov_ | | GAATTTACAAACTATCTGAATTAGATCAAGGAGTTTATCCGA | | | | | |
| 18.201118 | | ATCTTGGTTTGGAATTGTAGTTGCTCTATGAAGTTAT | | | | | |
| RAD_kmer_0002734_1_TGCAGGTGATTAGTGGGCTGCACCA | 115 | GGGAGATTGAGCTTTGAAACCAGTTCGTGAATGGTGATTGGT | G | K | G | K | GYSNP0780 |
| AAGCTATTGGCCATTGGGCTTCAAATCGTACTTAAACATA | | TTTCCATGATCATGAACGATGTCGGGTATTCTTAGTTC[G/T]A | | | | | |
| AAGGTGTAAAGAAATGGTTTGGGCCT_NODE_1_length_506_ | | GTGCACAACCAAGAGACATGGAGGTGGGATAGTTGAGGACA | | | | | |
| cov_70.616600 | | TGTCTGTATATATGACTTGAGCTTCAAAGACCACATTT | | | | | |
| RAD_kmer_0002749_1_TGCAGGTCTTTTATGTTTGTGGGCAT | 425 | GTTGTCGAGTTGATAGTAAATAAAGATAGGTGCGGTCATTTT | T | T | T | C | GYSNP0781 |
| CGGAGCGTGTTGTAGAAATCGTTGACCGTTATGACGATGA | | CGGAGGGACTTTCAGGGTTCGGACAGAAGTTTTGTTCG[T/C]A | | | | | |
| CTGTCTTCCCGATGCCTACAGCACT_NODE_1_length_520_cov_ | | TTGTTGCTCCGAATAAACAAATCTGGGTTAGTGCTGTAGGCA | | | | | |
| 150.194229 | | TCGGGAAGACAGTCATCGTCATAACGGTCAACGATTT | | | | | |
| RAD_kmer_0002757_1_TGCAGGAACGGTAAGCATTCACCTT | 162 | CCATATATCTATATACCCCTTCGATG GTCTCTCCCGTTAGA | G | R | R | R | GYSNP0782 |
| TTGTTGTCCTATTTTGGGTGATTAACTTAAGAACTCCATTA | | ATCAGTATAGAATGTCTTGTTTGTCTTTAAGCTAGTT[G/A]TG | | | | | |
| CATGTTGCAAGTTTTGTTACAGAAA_NODE_1_length_573_cov_ | | ATCTGAGTTGTTAATTCTTTCCCAACACCATCATCTATAGGTA | | | | | |
| 87.111694 | | TAGGTCCAACCTAGTACCAAATTGTAGAAATATGT | | | | | |
| RAD_kmer_0002763_1_TGCAGGGGCGTATTAACAATAAATA | 255 | TTGTGATTCGTGTCGGTGTCGGGATTTGTCTTAAAACTAAACC | — | G | G | K | GYSNP0783 |
| AAATAAATAAATTGTTTATATTGAAAAGATTATCTTGTAA | | ATGTCTCTTCACTTTTAGAAGATTAATAAGGGAATTT[G/T]TC | | | | | |
| GTATTCTAGAAGTTTAGGTTGTTAGT_NODE_1_length_503_ | | ATGAAAAAAAATGTTTAGAGATGACTTGGGGGTAAAAAGAG | | | | | |
| cov_96.986084 | | AGTTTTCTACTTGAGACCTATGGTGAATATAGAATTG | | | | | |
| RAD_kmer_0002765_1_TGCAGGTGATGTTGATGAGGAGGA | 92 | AACATTTATCAGCTGGACTCTGATACATACATAAGAATACAA | Y | C | Y | Y | GYSNP0784 |
| GAAGGCAATTGTCCTATACAAGGTAACAAGACACTAAGT | | CTATTATAACGCTCTTATGATAAGAACATATACAATGT[C/T]A | | | | | |
| AGAGTATTCTTATATTCTAGTGTGTAAT_NODE_1_length_451_ | | CGCAAGCTGCACCTGGCTAGATTGTATTAACAATTAAAATTA | | | | | |
| cov_67.339249 | | GTTTTCTTTGTACAAAACATACTCCTTTATTCCACAA | | | | | |
| RAD_kmer_0002766_1_TGCAGGGTACGGAAACGTGTACGG | 197 | CATCAGTTGATGCCGCTGGTGTTGATGAAGAACCTTGACCTT | A | — | A | G | GYSNP0785 |
| GGACGGGCCGAAGCGTTTCCAATCCGATTCCTTGACCGAT | | GTGTTTCTTGAGAACTAGGCATAATAATAATAATCGTG[A/G]T | | | | | |
| TCGAGAAGTCAACGACGTTTCTGCTGC_NODE_3_length_386_ | | AGAATATAGGATAGAATATGGCTGTGATTTTATTGATTGTTAT | | | | | |
| cov_54.321243 | | TGTTGAATCGTGGGTTTAGACTTTAGACTTTAGAGA | | | | | |
| RAD_kmer_0002766_1_TGCAGGGTACGGAAACGTGTACGG | 251 | AACTAGCCATAATAATAATAATCGTGATAGAATATAGGATAG | K | — | G | T | GYSNP0786 |
| GGACGGGCGGAAGCGTTTCCAATCCGATTCCTTGACCGAT | | AATATGGCTGTGATTTTATTGATTGTTATTGTTGAATC[G/T]TG | | | | | |
| TCGAGAAGTCAACGACGTTTCTGCTGC_NODE_length_386_ | | GGTTTAGACTTTAGACTTTAGAGAGGAAGGGCCGAAGGGTTT | | | | | |
| cov_54.321243 | | GAACTTTGAAGAGAAGAACATAAATCGTTCTTTGTT | | | | | |
| RAD_kmer_0002771_1_TGCAGGAGCAAAAGCATTTAGATGT | 231 | ACAAGGAATTTCAAACACCTCAATGATACATAATATTCCACT | C | C | Y | T | GYSNP0787 |
| TTGATAGGTGGATAAGCCAAGCTAAATACTCAGTGAACA | | AAAATTTTGTTCACTAATGATAAAACTTAAGTGCACAT[T/C]C | | | | | |
| CCATAAGTCATAGACAGCTTTAGGCAT_NODE_1_length_551_ | | CTTAAATTTAATTGCAAAAAAAATAACAAATTTGTCGAGAA | | | | | |
| cov_93.558983 | | AACCCCTTTAAAAATTCTTATGCGTGTTCTTACCCAT | | | | | |
| RAD_kmer_0002771_1_TGCAGGAGCAAAAGCATTTAGATGT | 319 | ATTTAATTCCAAAAAAAATAACAAATTTGTCGAGAAAACCCC | C | C | A | A | GYSNP0788 |
| TTGATAGGTGGATAAGCCAAGCTAAATACTCAGTGAACA | | TTTAAAAATTCTTATGCGTGTTCTTACCCATTATGCAT[A/C]TT | | | | | |
| CCATAAGTCATAGACAGCTTTAGGCAT_NODE_1_length_551_ | | ATGCGTAATGAAGTTTAAGAGCTTCAAGATAGTTGATGGTCT | | | | | |
| cov_93.558983 | | TATACAGTTGCTTAAATACATTATTATTAACCAATA | | | | | |
| RAD_kmer_0002772_1_TGCAGGGAATTTGTAAAATCTGTTA | 521 | GTAATTATGCTTGGAAGAAAAACATTAATAAAGTGGAAAATA | T | Y | T | Y | GYSNP0789 |
| TTCAAGGTGTTCTTCCAGGAATTGCTTTGAAAATTTTCCTC | | TAATATCTTGTTGCAGATCTCCTCTCTAAAGCAGATAT[T/C]G | | | | | |
| ATCATTCTACCTTCAATCTTAATGA_NODE_1_length_614_cov_ | | AGGTAAATCCTTCAAACTTAGACATCATCATTAAGATTGAAG | | | | | |
| 133.076553 | | GTAGAATGATGAGGAAAATTTTCAAAGCAATTCCTGG | | | | | |
| RAD_kmer_0002778_1_TGCAGGCCCGAAACTGTGAATTGCT | 432 | ACCCCATAGAGACGGGGAGGACCAGAAACTTTTAAAACTCTT | C | Y | Y | Y | GYSNP0790 |
| CGGCTAGAACAGGATTGGACATGCTGGAAAAACATTACG | | TCTAGAAAGTCAGATGGTGGGACCAAGAATAAGGTGGT[C/T] | | | | | |
| CAGATGCTATTGGTTTTGATATCGTTT_NODE_1_length_597_ | | CCGTCGCCAAATTTTACAACACCTGCTCGGTTTCGATCACCCA | | | | | |
| cov_110716919 | | TGTATTGTAGAAATTCGGTATATGAACCGAAATCCTC | | | | | |
| RAD_kmer_0002778_1_TGCAGGCCCGAAACTGTGAATTGCT | 522 | AATTTTACAACACCTGCTCGGTTTCGATCACCCATGTATTGTA | A | R | R | R | GYSNP0791 |
| CGGCTAGAACAGGATTGGACATGCTGGAAAAACATTACG | | GAAATTCGGTATATGAACCGAAATCCTCTTCACTATC[A/G]GG | | | | | |
| CAGATGCTATTGGTTTTGATATCGTTT_NODE_1_length_597_ | | TAAAAAGAAAACGATATCAAAACCAATAGCATCTGCGTAAT | | | | | |
| cov_110.716919 | | GTTTTTCCAGCATGTCCAATCCTGTTCTAGCCGAGCA | | | | | |
| RAD_kmer_0002783_1_TGCAGGTTAATGTGCCGCCAACCCA | 179 | GTAATTGAACGACTTGCTTGTCCATGCTTTATAAACTTCCACG | C | Y | Y | C | GYSNP0792 |
| TCAATTTTATACATATTTTTAACTTTCCTTTATATCATCTAC | | GCTATTTAACGAAATTACACGGTTTATGGACTATTTG[C/T]CG | | | | | |
| AAAATATAGAGATGATCCATTATA_NODE_2_length_358_cov_ | | AAAAGTAAATTATCCAACAGCCGAAACCCCTATGTTTTAGGG | | | | | |
| 19.871508 | | CGTTTGTTGTTATCAATTACACATGTGTAAAAATTG | | | | | |
| RAD_kmer_0002787_1_TGCAGGTAATTATTATTCTTATGTCC | 359 | GATTGTTCTTCTAGGTTTTGTTTATCGGATTTTTGAACTTGTGA | M | C | M | M | GYSNP0793 |
| ATTATATTTGGAATATTAAGATTTGAGATTATAGTTTGATT | | ATTTTGATAACTGATCCATTTAAAGTTTGGGATGGT[C/A]AGG | | | | | |
| CCGACTTTTTAACATTATCATATC_NODE_2_length_445_cov_ | | TTCGGGTAATTCGGATTGGGGTTAGTTCAGGTCGGATAGTTC | | | | | |
| 48.355057 | | GGTTTTCGGTTTAGATGTAAAATAAAATAAAATTG | | | | | |
| RAD_kmer_0002792_1_TGCAGGAGAAAAAGATAATCTTGTT | 214 | ATACATTACTTGTTGGCAATATAGAAAATATGTACATGTGCA | T | Y | T | Y | GYSNP0794 |
| CAAGGGCAAGGAGAAGAGATAAGAGAGAAAAATGCAGG | | TAGTGTAATATTTGCATTTTAAATAATTTTTTTGTGGG[T/C]GA | | | | | |
| TAGATATTAACCTTGCATAATCTAAGAT_NODE_1_length_331_ | | TTACAGGTTACGAGGCTGGTAAGAAAAGGTGCAAATAAGAT | | | | | |
| cov_69.861031 | | AACTTTGAGCATCGGTGATGGTGCAAATGATGTTGGG | | | | | |
| RAD_kmer_0002792_1_TGCAGGAGAAAAAGATAATCTTGTT | 270 | CATTTTAAATAATTTTTTTGTGGGTGATTACAGGTTACGAGGC | Y | T | Y | T | GYSNP0795 |
| CAAGGGCAAGGAGAAGAGATAAGAGAGAAAAATGCAGG | | TGGTAAGAAAAGGTGCAAATAAGATAACTTTGAGCAT[C/T]G | | | | | |
| TAGATATTAACCTTGCATAATCTAAGAT_NODE_1_length_331_ | | GTGATGGTGCAAATGATGTTGGGATGATTCAAGCAGCTCATG | | | | | |
| cov_69.861031 | | TTGGTGTTGGAATAAGTGGTTTAGAAGGAATGCAGGC | | | | | |
| RAD_kmer_0002792_1_TGCAGGAGATTTACTATTTTCTTCA | 320 | GGCCCTGCTACTTGTCATCATATAGACAGTGGGCACACGCGT | C | — | G | G | GYSNP0796 |
| GCTGATGCCTTTTTAAAAAATATATAATATATTATTTGCCA | | CGCTTCTAGTCCGGATTCTGACTTAGAGGCGTCCAAAA[G/C]T | | | | | |
| TGCTGATATACAAGGAGCCCATGTT_NODE_1_length_599_cov_ | | CAGCTAGAAGAACGACCTGGATCACTTTTACACTTAATGGGT | | | | | |
| 69.490822 | | TGACTGGTCATCTAGTGGAACCTTCATGTCAACAGTT | | | | | |
| RAD_kmer_0002797_1_TGCAGGCTGGTTTGTTCAAGGGTTT | 363 | CCATTCTATTAAAAAAATATTCTCAAATGGATTTTTATATAAT | G | A | G | G | GYSNP0797 |
| GAAGAACTCGCCGTTTACAGACATTTGTCTAGCTAATATA | | ACTTCAGTATCACAAACTGGTTAATTTTGGTGATTAC[G/A]AC | | | | | |
| CATCTAAGCGGGTCAACTGGTTCGAG_NODE_1_length_583_ | | AATGATACAGAATTAAACTTGAAATATTACACATACATTCAG | | | | | |
| cov_87.132072 | | AAAGGAATAGTGCATACACTGGTTTGGCTTCGGCTG | | | | | |
| RAD_kmer_0002801_1_TGCAGGAAATGGGTCGATCAAAGG | 280 | AGCCTAAAAAAGGGGGGAGGGGGGCGCGAAGCTCAATATAT | — | G | R | G | GYSNP0798 |
| TATCATAGGAGTTGTTTTAACGGATGAACCGATTCAAAAA | | ACAAAATTTTCAGACGAAAACGGGGAAATTTACACTACT[A/G] | | | | | |
| GTATGGTTTGTTGCTCAAGCCATTAGA_NODE_1_length_546_ | | AGCCGAAAAACAGGGGGGCCGGGGCACCCCCTCCCCCATCT | | | | | |
| cov_57.593407 | | AAAGCTTCGCCAATGCACATGAATAAGTAAAATGTGTAA | | | | | |
| RAD_kmer_0002802_1_TGCAGGTGTTACGCATGCAAGAAAA | 298 | GAAGACCAGTTTTCACCAAATGCAAGCAGGACTTTTCCCTTA | A | G | A | A | GYSNP0799 |
| CCAACATCAAGTGTTGAGGCTGACATAGTAGGTGGTGGAT | | TAACCGTATACTGGACCACTACAAAATGTATGTAAAAA[A/G] | | | | | |
| CAATCACCTGCTCTCATGCTCAACCA_NODE_1_length_637_ | | TATATTTAAGATTTGAGATACCATAAAACTATAAATTCACCA | | | | | |
| cov_164.094193 | | CATGCCTAAAATCTTCCAATTAAAGTTAAAGATACCTT | | | | | |
| RAD_kmer_0002802_1_TGCAGGTGTTACGCATGCAAGAAAA | 507 | TTAACTCTGTCACCTGCCAAATGATGGTTTTCATTACACAAAT | G | A | G | R | GYSNP0800 |
| CCAACATCAAGTGTTGAGGCTGACATAGTAGGTGGTGGAT | | GAAGATATGAGTAACAAACACCATATAGAATGCAAGT[G/A]T | | | | | |
| CAATCACCTGCTCTCATGCTCAACCA_NODE_1_length_637_ | | TATATAGCTATATACCTTCTTTGAAAGTATAACGTTTTGATGA | | | | | |
| cov_164.094193 | | TGCAGTGGACGCTTCCTGTTTTGGTTGAGCATGAGA | | | | | |
| RAD_kmer_0002806_1_TGCAGGCCACATCTGGAAACAAATC | 427 | CAGTAGGATAATAAGATGCAGGCACAAGTCTTCAGGCCGTCA | T | A | T | — | GYSNP0801 |
| CCTGTTAGTTTATAATTACTGAGTGTTGAACAGATATATA | | TTTCTTCCTCCTGCCTCCAATTCCATCACAACCCTCAC[T/A]AC | | | | | |
| TATACATATATATATATGGTGAATAC_NODE_1_length_536_ | | ATCTTCAAACCTTTCTTTTCCTAATAAATTCCCATGTCAGTAG | | | | | |
| cov_78.117538 | | TATTCACCATATATATATATGTATATATATATCTG | | | | | |
| RAD_kmer_0002809_1_TGCAGGCTGCAATTACATATATTGG | 85 | GTCGATACACTTCTGTTTTATGTACCTGAAAATATGAAGGCA | A | A | W | W | GYSNP0802 |
| AAGAAATGAATGTGTACATGATTTCAGTCACCATGATTCT | | CGTGTGAATCAAATGAAGATTAAAATCAAAACGAAAAC[A/T] | | | | | |
| CTTTTCACTGATATTTTCAAATTTAT_NODE_1_length_611_cov_ | | AAAATAGTGGAAATAAAAAGAACCTTTGTAAGAGATCTTGGT | | | | | |
| 99.869064 | | AGAGATAAGGCAACTCCAACAACTTAAACGCGATTACA | | | | | |
| RAD_kmer_0002810_1_TGCAGGCGGCGCCGCGGGGGACCA | 218 | TTCAGTCGTCTCAGCAACAATTTTTATTCTGATCGAGCCTTGC | T | C | T | — | GYSNP0803 |
| TAGTCTAATTAGCGGTGGCGGCGCGGCGGCAAGGAGCGC | | ATTATTGGCGGATAGTTTTTCTTTCGGTTGCCGTTTT[T/C]CCT | | | | | |
| TTGATGGAGAAGCGGAATCAACAAGACG_NODE_2_length_ | | TCAAAGTCTCACAACAATCTGTTCTTCCTCGTGAACCATTAAT | | | | | |
| 534_cov_52.234081 | | ATTGTTTGGAATATAGTGTCCGACAAAATCATTA | | | | | |
| RAD_kmer_0002811_1_TGCAGGTTTAAATGAACTTTTTTTAT | 154 | CATGTAAATGCTATATGTTGCTACAAGGATCCATAGGCACCA | T | T | C | T | GYSNP0804 |
| GATTATTATAACATATTTCAAGAATCAATAGTGCAATTAT | | GAGCGAAGCAAAGATGCTTCTATCCGGTCGTTCTTATT[C/T]G | | | | | |
| AAGATATTATTCATTTTTCCTTTTT_NODE_1_length_641_cov_ | | GCTTATAAGCACATGCAAGCCCGGTTAGACTCGCAACATAAT | | | | | |
| 119.723869 | | AGGGGGGAAGATGTTATCGTGAGTGACCAGACTCTGA | | | | | |
| RAD_kmer_0002811_1_TGCAGGTTTAAATGAACTTTTTTTAT | 449 | TGTTTTTGGTGTATGATCAAACAATCAAACGTTGTATACTTCA | T | C | C | T | GYSNP0805 |
| GATTATTATAACATATTTCAAGAATCAATAGTGCAATTAT | | GTTATCGTTTTGTTTATTGATCATTCCAAATGGCAAG[C/T]TTT | | | | | |
| AAGATATTATTCATTTTTCCTTTTT_NODE_1_length_641_cov_ | | CTCCATAGCCATGACGGTAAATATTTGTGGCGCTTTTGCAGG | | | | | |
| 119.723869 | | AACCAGAGGACGTTTCGTCCAAAGAAGAGTGCACC | | | | | |
| RAD_kmer_0002813_1_TGCAGGTGTTCCTTTGAAATCAATT | 586 | TAATAATCCTCCTAATGGTTAATGGACACTTAGTTAGCTAAAT | A | A | T | A | GYSNP0806 |
| TGGTTTATGGTTGGTCCACTTGGTTCCTGGTCTTATATATG | | AATTTATTCAAAAAAATTTTAAAAATACAAAATTACT[T/A]TA | | | | | |
| CTCCTTAAAAAATTTATTGCCGAAG_NODE_1_length_654_cov_ | | ACTTCGGCAATAAATTTTTTAAGGAGCATATATAAGACCAGG | | | | | |
| 79.388382 | | AACCAAGTGGACCAACCATAAACCAAATTGATTTCA | | | | | |
| RAD_kmer_0002814_1_TGCAGGTAACAATTACAGGGGTAA | 361 | TAAGATAAGTTAGAACCCAAACAGGCTAAATCGACCCATTCA | C | G | G | — | GYSNP0807 |
| GGGACCATCTGCATCCACCAGTTCGATCACTTCTAGAAGC | | TTAAGTAAATTGGTTGATATTGTCTTCTCTATTGTAAA[G/C]TA | | | | | |
| ACAACCATGAAGAAACTATCATCTTCA_NODE_1_length_599_ | | AGAATTAAATATGGTAGATCCAGAAGCTTAAGTTGACCAACC | | | | | |
| cov_99.215363 | | CGGGGTTGAAAATGACTTCCTGAAAGTATTTCCTCT | | | | | |
| RAD_kmer_0002831_1_TGCAGGCTGTGATATTTGAAGCTTT | 473 | GAACTGGATTCAAAAACCGGATAACGTCATAAATAACTTAGA | T | T | T | C | GYSNP0808 |
| GTCTATCCAGCATCCTCCATTGTTTTCCATACACTCATTTG | | AACCAAATATATGAATATATAACCAATTAGCAATACTT[T/C]A | | | | | |
| TTTCAATTCCTGAAAGCAGCATATA_NODE_1_length_609_cov_ | | TTTTCTGTGCGGATTTACAAAAGTATTAAACTATGAAGCTTTC | | | | | |
| 103.477829 | | GAATACTGAAGTGAAGCACGTGATGCATATATGCTG | | | | | |
| RAD_kmer_0002833_1_TGCAGGATGCGGTTAACCTTTTTGA | 482 | TCAATAAAAAGGTACCCTAGCGCCTAAATTTCATCTTCTTGGC | A | G | G | G | GYSNP0809 |
| TGAAATGATCGATAGAGGTATTCAACCGAATACTGTGACA | | CTTAATAGTTTTTAGTGCTATCATTGTTCGCGAGTTC[G/A]GCT | | | | | |
| TACACTGCTCTTGTATGTGGCTATTG_NODE_1_length_612_ | | CAACTCCCTTCGATATCATCTCATCAACAAGTGTTATTGCACG | | | | | |
| cov_122.586601 | | CTGCCTATATCCCATAGAGCAATAGCCACATACA | | | | | |
| RAD_kmer_0002844_1_TGCAGGAATGTTTATACAACAAGGG | 286 | ACCCGTTTTGACCTACCCGCTCTTTTTAACACTGCTATTACAA | G | R | G | 5 | GYSNP0810 |
| CTTACCTCATTTGTATGCTCTCTTTTTTTATATGATTATTTT | | ACAATGTAATAACCTCACCTGAAAAACACTAGAAGTC[G/A]A | | | | | |
| GTAATTCTACATAAATGAATGTTT_NODE_1_length_632_cov_ | | CGAAGACTCTCCATGCTTAGAAAATCCAAACCCACAGCCTTT | | | | | |
| 168.085449 | | GCATAACCTTATGAGTATATTTGTTACAGGAATCCAT | | | | | |
| RAD_kmer_0002847_1_TGCAGGACTATGGTCCGCCGCCACG | 502 | GGAAACCAGAAGCTCATACTGAAAAAACAATAATATATAAA | A | G | A | G | GYSNP0811 |
| CTGTTAATTAGTTAGTTATTTTCTTCACCGTAGACGTCG | | TAATAAATAATAATAGGATAAACGTACTTCGCTCGATCA[A/G] | | | | | |
| TGTAGCCGCATTGCATGCCTCTCGC_NODE_1_length_647_cov_ | | AAATCCAAACGTAAACCAACTAATCCATATTTTGCTTGATTTT | | | | | |
| 75.074188 | | GCCATCCATAGTATTCAATGACACATCCCGTAATCCT | | | | | |
| RAD_kmer_0002848_1_TGCAGGTTGAGCAAAACAATGCAA | 356 | TCATATACATTTATAGTTATTTTCATCTTGAGCTTGGCTCATG | C | A | M | M | GYSNP0812 |
| AAATTAATCACCATTTTCCTTCCATACTAGGGGTGTAACG | | TGTAGTTTACTAACCTCGATATATGAAGCTCGGGCTC[A/C]AG | | | | | |
| AGCCGGACCACTCGTGAGCCACTCGAT_NODE_1_length_499_ | | CTCGTTTATTGAATGAGCCCCGTATAAGCTTGAGCTCATTTAA | | | | | |
| cov_180.006012 | | CCTTGGCTCGATTCGAACTTTTAACGAACTGATAT | | | | | |
| RAD_kmer_0002849_1_TGCAGGAAACGTTAACAAGATTCAC | 391 | AGGGGTCGTTCGGGACTAAATCGCATATATACTCATCTATCA | C | C | T | C | GYSNP0313 |
| TTTGTGCAACCAACCTATTAAGCAAGTAAATATACAAAAG | | AAACAAATTAAAAATTCATGAAATGCGGCCGCAACCCA[T/C] | | | | | |
| CAGCTATAATCAACTGACAAGTCTAG_NODE_1_length_505_ | | CCGCGCCATCTAGGTACGCCACTGCAGTTAACTGTGAAAACA | | | | | |
| cov_97.380196 | | TATGGTCCTAGACTTGTCAGTTGATTATAGCTGCTTTT | | | | | |
| RAD_kmer_0002850_1_TGCAGGGTTTGATCTTGATAAGGTA | 227 | CTAAGTTCACGGGTATAGGTACCCATTTATGGTCTTATATGGT | A | — | R | A | GYSNP0814 |
| CACCCGAAATTTCACGTACTGATTTGCTTAGGTGGTATGA | | TGAGTATTACCTCGGGGATGCCCATTACCCGAAACCC[G/A]AG | | | | | |
| TGAATGGGTTGGGTAACATGTCAAAA_NODE_1_length_405_ | | CTTAAACTCACGTAAAAAAAGGGACTTGAAATATCAGATACT | | | | | |
| cov_38.854321 | | CAACTACATTTCTAATAATTTATAATATTATTAATT | | | | | |
| RAD_kmer_0002854_1_TGCAGGAGGTGTTTTACAGCTTAGT | 179 | CTAAGCTGGTAACTTGTGTTTTGACACTGTTTATCATATAAAT | A | A | T | T | GYSNP0815 |
| ATCTCAACTTATAATGAATAATCAACGAAAGCGTCATTAA | | GCGAGTTGAAATCAATATAATATGTTTTAGTATTTAG[T/A]TA | | | | | |
| ATATATGTGCTTCTGTTGGAATTTGT_NODE_1_length_496_cov_ | | AAAGATTTTGCTTCTCGTTACCTGGTTACTTAGGGTGTCATAT | | | | | |
| 118.100807 | | AATCAGTTTCATATCAGCTGCAGGCCTGCAGCAGA | | | | | |
| RAD_kmer_0002855_1_TGCAGGCATGAAAATGATCTACAGC | 225 | TGGGATTTCAGGTAAAGCGGGGGACACCTCAGGGTTCCCGCA | M | M | A | M | GYSNP0816 |
| ATCAGCAGAAACTTGACACTTGCCACAAGGTTGGTCCATC | | GGGCACCACCCGAGTCTCATGTTAGGACCCATGCAAAG[A/C] | | | | | |
| GTTGATGTTTCTGTTTTCATGATTCG_NODE_1_length_596_cov_ | | AAACTCATTCTTAGCTGCTATCTGTCCCGCAGCTGCCAGCATT | | | | | |
| 123.526848 | | GTTGCCTTTAACTCTTCAAGGCGAATCTCCCACAACC | | | | | |
| RAD_kmer_0002858_1_TGCAGGGCTGTTGCATATATTCCTTT | 85 | GCAACGAAAACCGTCATAAAACTGCACATAAAACTTACTGTT | R | A | R | A | GYSNP0817 |
| AACAATTCTTGCAGTTCCTGCATCAGTACTTACTGTAAGA | | TTGCCAAGAAGGAATGCTGCAGTTGCACCTATCACCGC[A/G]C | | | | | |
| TCTCAACGCCACACACATGCATTTA_NODE_1_length_638_cov_ | | CAATAGAGTCAGCAAGAAAACCCATAGGCAAACCAAATAGA | | | | | |
| 85.957680 | | TAACCACCACCCAGCTGGAAGAAATACCGGTAAATACA | | | | | |
| RAD_kmer_0002858_1_TGCAGGGCTGTTGCATATATTCCTTT | 152 | CACCTATCACCGCACCAATAGAGTCAGCAAGAAAACCCATAG | C | T | Y | T | GYSNP0818 |
| AACAATTCTTGCAGTTCCTGCATCAGTACTTACTGTAAGA | | GCAAACCAAATAGATAACCACCACCCAGCTGGAAGAAA[T/C] | | | | | |
| TCTCAACGCCACACACATGCATTTA_NODE_1_length_638_cov_ | | ACCGGTAAATACATACATATATACATAAATATATATACACAC | | | | | |
| 85.957680 | | ACACACACACAAACATAACAAAATAACTATCCAATGGT | | | | | |
| RAD_kmer_0002859_1_TGCAGGGCCCGCATCATCCCTCCCC | 161 | AACGACTCGGAGTGTCTCGTGCTTTTTAAATCCAAGATCAAT | T | T | C | — | GYSNP0819 |
| CCGTTCAGAGTCTTCATTCATCGGACACCATCCGGCCTTA | | ATATTAAGCTTTAAACTGATTCTCTCTGTATATTCTTA[C/T]AC | | | | | |
| CATAACTTGATAAAGCTGTCTTTTGC_NODE_1_length_550_ | | TTTGAATCAACACATAACACTCTCAATGTCAAGCTTAAATCTT | | | | | |
| cov_82.063637 | | TCTTGAGTCAGGCTCGAGATTGTATCATGATGGCT | | | | | |
| RAD_kmer_0002859_1_TGCAGGGCCCGCATCATCCCTCCCC | 243 | CTTTGAATCAACACATAACACTCTCAATGTCAAGCTTAAATCT | G | C | G | — | GYSNP0820 |
| CCGTTCAGAGTCTTCATTCATCGGACACCATCCGGCCTTA | | TTCTTGAGTCAGGCTCGAGATTGTATCATGATGGCTA[G/C]TG | | | | | |
| CATAACTTGATAAAGCTGTCTTTTGC_NODE_1_length_550_ | | GACTCCATCTAGCTATTTTTGAGCTACGGTTAAGGAGGTCGAG | | | | | |
| cov_82.063637 | | CCAACACATGAAATCGGGTCATTTAAGTTGTGCCCG | | | | | |
| RAD_kmer_0002859_1_TGCAGGGCCCGCATCATCCCTCCCC | 446 | TCTAGGGTTACAGCGTGCTTAATTCAGGATACGTTTTGAAATT | T | T | C | — | GYSNP0821 |
| CCGTTCAGAGTCTTCATTCATCGGACACCATCCGGCCTTA | | ATTTGTAGCTGGTTTATTTGAAGCCAGAACGGGCCAA[C/T]TA | | | | | |
| CATAACTTGATAAAGCTGTCTTTTGC_NODE_1_length_550_ | | ATTGTACATACAGTCGGTATGTTTTGTAACTAGGGCCGCAAA | | | | | |
| cov_82.063637 | | AGACAGCTTTATCAAGTTATGTAAGGCCGGATGGTG | | | | | |
| RAD_kmer_0002861_1_TGCAGGTATGAAATATTTCTACATC | 369 | CTTGATTCTTATCAAAGCTCAGTATACCAAAAGAATCATAAT | R | A | R | R | GYSNP0822 |
| CCCTCTTTGTGAAATACGGAGTACCTTTATTCATACCATGT | | CGAAGAAAGATCAAACTCACAAAAAGTCCTGAACAAGT[G/A] | | | | | |
| TTGCTTCTCGTACACATGATCGTAA_NODE_1_length_466_cov_ | | GTATACTTTATCCCATGAGATCTTAAAAGGAGTTACGATCAT | | | | | |
| 127.952789 | | GTGTACGAGAAGCAAACATGGTATGAATAAAGGTACTC | | | | | |
| RAD_kmer_0002865_1_TGCAGGTTGGAACATTGTATCAACG | 187 | AGTCTGGTAAGTGGTAACTAAGTAATGAAACCCAATAAAAG | A | — | W | A | GYSNP0823 |
| TCAAAGTTGCATTTTATGTACCAAAAGTCGTATTGTCACC | | GTAGCCTACGCAAATAAAGGTTGTACAGAAGGGCAAAGA[A/T] | | | | | |
| ACTTTCATACCCTAACACAATAACTT_NODE_1_length_460_ | | GTTCATAAACATTGTCGCAAAAATATTATATATCTTTTACAT | | | | | |
| cov_43.65865 | | TTTAGATATTCAAACTGAATTTCTTTGGTGTTTACCGT | | | | | |
| RAD_kmer_0002877_1_TGCAGGTTTAGAATGATTAAAAACA | 373 | TGTTTGCCTTCTGAAATGGCAGTCTGATGATGTTAATCAAAAT | W | A | T | A | GYSNP0824 |
| ATGAAAATGATATTATTTTGATGATTCAAGAAACCTTTTT | | AATCCCATTTGTTGTTCATATTTAAACTATTATAACC[T/A]AAC | | | | | |
| CCTATGCTTCCTTTTCTTGTTTCTTC_NODE_1_length_504_cov_ | | CTGCTTATATCAAACTACATGAATTTCATGTTGCAGAGATTGA | | | | | |
| 52.277779 | | GGCGCTTAGCTCAAAATAGGGAAGAAACAAGAAA | | | | | |
| RAD_kmer_0002881_1_TGCAGGCTTTTAGGTAACTTTTTCA | 95 | GCAACATGACATTCTGGTGATGTTTAATTAGAAAGTTTGGCT | T | C | T | C | GYSNP0825 |
| ATAGCTGGGATGTTCGTTCAAGTGCATAAGAATTTGAGTA | | GAGACATTTAAACAAACATCTGGTATACATTAAGAAAC[T/C]C | | | | | |
| GGGTTTGGTCAAGAGATAAAGAAGCA_NODE_1_length_326_ | | ACAAGCGAAAACTGAATTTCAAAAGTCATTTTAAGACTATTG | | | | | |
| cov_46.248466 | | ATTCATTTTTGACATATCACAATAAAATATAAAAAAT | | | | | |
| RAD_kmer_0002881_1_TGCAGGCTTTTAGGTAACTTTTTCA | 237 | ACAATAAAATATAAAAAATCCGGTAAAAACTGTTACTATGAA | A | — | A | G | GYSNP0826 |
| ATAGCTGGGATGTTCGTTGAAGTGCATAAGAATTTGAGTA | | GAGTAATAGCATTCCAAATATCCAATTTGGGTATATCA[A/G]T | | | | | |
| GGGTTTGGTCAAGAGATAAAGAAGCA_NODE_1_length_326_ | | TCCCATTATTCCTTTTTAGTATTTGCTTCTTTATCTCTTGACCA | | | | | |
| cov_46.248466 | | AACCCTACTCAAATTCTTATGCACTTGAACGAACA | | | | | |
| RAD_kmer_0002890_1_TGCAGGATGGTACGGAAGTTGAATA | 227 | ACATATGGCGAAAATTTTTAAAAATTTGCAAAAACATATAAT | T | A | A | — | GYSNP0827 |
| TTGGGCCGCAAGGGGAATATGTCATAGAATATTAACCAA | | ATTGTGAAAAAAAATAGAAACTATGCAATATTTTTTTT[A/T]A | | | | | |
| GTTTTAAAAGTAATAGGTCACTAAAAA_NODE_1_length_495_ | | AAAAATGTCAAAGTTCTTATAAAATTGCGAAAAATAGTTAGG | | | | | |
| cov_86.795959 | | TGATAGTTTATTTTTAATGGTAGAAAGTAATAATTGC | | | | | |
| RAD_kmer_0002894_1_TGCAGGTCCACGCCCACCTCCTCCA | 343 | ACCAAATAACGTTTCTATCATTTCTTCACTAAACCTGTAACAA | C | C | Y | Y | GYSNP0828 |
| CCAGGTGGTGCACTGCCGCCACGACCACCAGGTTCCAGCC | | ATTAATCATGTTATGGTATCTTACCATATAATGAATA[T/C]AA | | | | | |
| TAAGACCGCCACGCCCGTCAGAGGGA_NODE_1_length_568_ | | GACAAAGAAGCTAAACAAATGAGAACTTACTGAAATGACCC | | | | | |
| cov_159.220078 | | TGATTTTATCTGATGCCAAACCATCGACTGATCAGGG | | | | | |
| RAD_kmer_0002894_1_TGCAGGTCCACCCACCTCCTCCA | 480 | TGCCAAACCATCGACTGATCAGGGTTAGCCATAACCTTATCC | G | G | R | R | GYSNP0829 |
| CCAGGTGGTGCACTGCCGCCACGACCACCAGGTTCCAGCC | | CAAAAGAACGGCTTTAGCTTAGCTTTAGAAGCATCGGC[A/G]T | | | | | |
| TAAGACCGCCACGCCCGTCAGAGGGA_NODE_1_length_568_ | | CATCACCACTTGAATGACTAGATCCCTCTGACGGGCGTGGCG | | | | | |
| cov_159.220078 | | GTCTTAGGCTGGAACCTGGTGGTCGTGGCGGCAGTGC | | | | | |
| RAD_kmer_0002898_1_TGCAGGAACACATTTCACTCATCAA | 214 | ATGATTACCAAAACTGTATCGTTTAATTAAAATGATAATTCCT | T | A | A | A | GYSNP0830 |
| ATACATTATTATTATTATTATTATTATTAAAATGAATATCA | | TTAATATAAAATGATTTAGGACATTTTAAGCTTTTGA[A/T]TA | | | | | |
| AAATAATGAATTAATAAAAATCCTA_NODE_1_length_637_cov_ | | CACATTAAGCGACTTTTGACCCGTTTCATTGTTAGCTAATATT | | | | | |
| 86.781792 | | AGACATTTGACCCTTATCAAAATAATACAACCCGA | | | | | |
| RAD_kmer_0002900_1_TGCAGGTCTTTAACATATTCATTAG | 526 | AAACTGAGTGTCATTTGAGATGACTTGTAATTGTGTAAATCA | A | G | G | G | GYSNP0831 |
| GATCATTGCGCAACCTATTTTGACCCGAGCACGTTTCATC | | TGTTTTCTGGTAACTGGACTTCTTTTATGTGGATGAAA[G/A]A | | | | | |
| CCTCCATTTAAAAGGACCAAACTTGT_NODE_1_length_616_ | | GACTTGACTTGTTCATATGCACAGACAAGTTTGGTCCTTTTAA | | | | | |
| cov_97.777596 | | ATGGAGGGATGAAACGTGCTCGGGTCAAAATAGGTT | | | | | |
| RAD_kmer_0002905_1_TGCAGGTAGCCCTTCATGGGTTCGA | 131 | GATTTTCATTCTTTGATTCCACACAGATGTGATACTTGTTTA | C | T | T | T | GYSNP0832 |
| ACGCACGCGGTAACACCGCTGCCACCCCCTTGCGGGTAGG | | TAAATCTCTTAGCTTCACTGCTATCTTGATTTATGAT[T/C]CCT | | | | | |
| CTGGCAGGTACCCGTAGTAGGTTCCT_NODE_1_length_562_ | | TACTCTAAATTGGAGATGTTTCTGAGTGATTTTATTATGTTGC | | | | | |
| cov_78.507118 | | TTAGAACCTCCAAATTATGCATGTCTGGCTTAAC | | | | | |
| RAD_kmer_0002905_1_TGCAGGTAGCCCTTCATGGGTTCGA | 462 | TACAAACCACAAAATCACTAGCAGCACTACCCACAAATACCC | T | W | W | T | GYSNP0833 |
| ACGCACGCGGTAACACCGCTGCCACCCCCTTGCGGGTAGG | | TCACACAACCTCTCCCTCTTTTTCTCTCTCTCACACAC[T/A]CA | | | | | |
| CTGGCAGGTACCCGTAGTAGGTTCCT_NODE_1_length_562_ | | CAAGTCAACACACACACAAAGGTATGCGGGTATAGGAACCT | | | | | |
| cov_78.507118 | | ACTACGGGTACCTGCCAGCCTACCCGCAAGGGGGTGG | | | | | |
| RAD_kmer_0002906_1_TGCAGGTTGCTGCAGGGATTATTTA | 195 | GAAGTTATTATATATAGTTAATAATGAGTCCCAAAATGCAAA | G | G | G | A | GYSNP0834 |
| CCCCAAAAATATGTACTGCTGAATATGACCCAAAAAAAG | | TCCGAACACCCCTTAATAACTTCTGTTTAAATGGAAAT[G/A]A | | | | | |
| TGATATCACAATAAAAGTTGAGGGAGT_NODE_1_length_515_ | | ACCATCCCTGTTCAATTTCACCAGTGCAGGCAAATCGAAGAA | | | | | |
| cov_104.120392 | | AAATACCTATTTTAATTGATGCTGAAAGACCAAGGGA | | | | | |
| RAD_kmer_0002908_1_TGCAGGGTCAATTAATCCGGTCCAC | 523 | GGTTCCTTAGTATCATTATTGTATTGAAAATGTACAAGTCCCT | W | W | T | T | GYSNP0835 |
| ATACATCCTAGGGCTTCAGAGCTTATGTTTCTTGTTTCCGG | | TAGGAAACACAAAAATATCACCAATTTGTAGGCTTTG[T/A]TT | | | | | |
| GTCTCTCCAAGTAGGGTTTGTGGAT_NODE_1_length_608_cov_ | | AAATAGCTTGTTACTAGTATCCACAAACCCTACTTGGAGAGA | | | | | |
| 137.523026 | | CCCGGAAACAAGAAACATAAGCTCTGAAGCCCTAGG | | | | | |
| RAD_kmer_0002909_1_TGCAGGAAGCTACAGATAATGAAC | 156 | AATTTTTAATACAAGCTAACGATTTAAAAAAAAAAAAAACGA | C | C | C | G | GYSNP0836 |
| CCATATATTTAACAAATAACATGTGCACTGGATAAACAGC | | ATCTGCTCTAACTGCCCTGACCAAACCGTTTTGGCCCG[C/G]C | | | | | |
| GGAATTAGTGTCTCCTTTCACATAACT_NODE_1_length_502_ | | CGGCCGGCCCACAACTAATTAGGCCGGTTTGGTTAGCCGTTT | | | | | |
| cov_116.695221 | | TTCAAACTGTAGTTAGTGGCTCGGCCTAGAATTATTT | | | | | |
| RAD_kmer_0002909_1_TGCAGGAAGCTACAGATAATGAAC | 322 | ACCTGCTTAAAGGACAGTGAACGCCCTTGCTAGTAAAAAAGT | A | A | T | T | GYSNP0837 |
| CCATATATTTAACAAATAACATGTGCACTGGATAAACAGC | | TAGCCTGAAGAACTATATTTTTATTTTGATGAAGCTGA[T/A]G | | | | | |
| GGAATTAGTGTCTCCTTTCACATAACT_NODE_1_length_502_ | | TACAATGATCTGCAGGCAAGGTGTCCTGTTATGTTGTACAGA | | | | | |
| cov_116.695221 | | TTAGATAGATATTCAATTAATTTAGCTTTTTTCTGTA | | | | | |
| RAD_kmer_0002911_1_TGCAGGCGGCGCAACGGAGGACCG | 347 | CCCAGCTACTCTTGATTGGCATGGAATTAATTTGAATTAATTT | R | — | A | A | GYSNP0838 |
| AAGTCCAATGTACGGCGGACCGAAGTCGGCGGCAATAAT | | TTGGTTCAAAACCAAAGCAGAAGCAACATGCTGCTC[A/G]TC | | | | | |
| AAAAAAACAAAGAGGAAGGCGGCGGCAA_NODE_1_length_ | | CAAGCCTACGTGTTTTTTGGCCTTTGTTTTAGGTATTCTACAT | | | | | |
| 562_cov_68.583633 | | GGTTGGTTCTTGTTTCATGGCTGACAAAGAAAAG | | | | | |
| RAD_kmer_0002911_1_TGCAGGCGGCGCAACGGAGGACCG | 412 | AGCAACATGCTGCTCATCCAAGCCTACGTGTTTTTTGGCCTTT | R | — | R | G | GYSNP0839 |
| AAGTCCAATGTACGGCGGACCGAAGTCGGCGGCAATAAT | | GTTTTAGGTATTCTACATGGTTGGTTCTTGTTTCATG[G/A]CTG | | | | | |
| AAAAAAACAAGAGGAAGGCGGCGGCAA_NODE_1_length_ | | ACAAAGAACAAGTATTTTGATGATGCTACGGCACCATCTCAA | | | | | |
| 562_cov_68.583633 | | CTGCCATGCCACTTATCCATCGTACTTCTCAAGCT | | | | | |
| RAD_kmer_0002913_1_TGCAGGTAACAAAGTAATATGAAA | 87 | AATCTAGTGTTTTCAAGTCACAGATAATCAAGTTCAAGTGTTT | G | G | A | — | GYSNP0840 |
| ATATTTAAGTGATGTTTGGTATTGCGTTTTGGAAGTGATTA | | GGGTATAGTTAAAACTTTAAACTGATTCTGATTATCT[A/G]AT | | | | | |
| TGTGATAGGAAATATTCAGAACAAGA_NODE_1_length_481_ | | TTTTTACGGGTCAGATAATCGATTTAGGAGTAGCTGCAACAA | | | | | |
| cov_127.507278 | | ATTCGAAAATACAGGTCAGATAATCAATTTAGGAGT | | | | | |
| RAD_kmer_0002913_1_TGCAGGTAACAAAGTAATATGAAA | 266 | AACTTACAGTATCTTAAAGAATCCCACTCAAAACATCTGCAC | C | C | T | — | GYSNP0841 |
| ATATTTAAGTGATGTTTGGTATTGCGTTTTGGAAGTGATTA | | ATCCAATACGGGTCAGATAATCGATTTAGGAAACTGCA[T/C]C | | | | | |
| TGTGATACGAAATATTCAGAACAAGA_NODE_1_length_481_ | | AAATTTGAAAATACAGGTCAGATAATCAATTTAGGAGTAGCT | | | | | |
| cov_127.507278 | | GCAAGTCGGCAACAAACCCGAAAACCTTCAATATCTT | | | | | |
| RAD_kmer_0002913_1_TGCAGGTAACAAAGTAATATGAAA | 383 | AGTAGCTGCAAGTCGGCAACAAACCCGAAAACCTTCAATATC | T | T | T | — | GYSNP0842 |
| ATATTTAAGTGATGTTTGGTATTGCGTTTTGGAAGTGATTA | | TTAAAGAATCACACTCAAAACATCTGCACATCCAATAC[G/T]A | | | | | |
| TGTGATACGAAATATTCAGAACAAGA_NODE_1_length_481_ | | CCTTGTGCAACCTGTTCTGTTACAATTGGTTGTCTTGTTCTGA | | | | | |
| cov_127.507278 | | ATATTTCGTATCACATAATCACTTCCAAAACGCAAT | | | | | |
| RAD_kmer_0002922_1_TGCAGGCGTGGGATGGCGTTTGTTT | 87 | TTTATACGGAAAAAAAAAATCAGCACTTCCATTAGGAATTAG | Y | Y | C | C | GYSNP0843 |
| TTGTGTGTGTGGTTGTAGGAGGGAGGGGTGGGGGTTGGA | | TGTTGGATTCTTTGCGCAACTCCGTGAGTTTTGAATAG[C/T]TG | | | | | |
| GGTGAGGAGTTGGTGACGGGCAAGCAA_NODE_1_length_569_ | | CTGAAGAAAATAATGGAGTTCATATCAGTTCTACTACAATAT | | | | | |
| cov_98.437607 | | TAGCTTATGTGTAATGGAAGGCCACATTTGTACATG | | | | | |
| RAD_kmer_0002924_1_TGCAGGATTGGTAGGGAGGCCATTG | 525 | AAGAAAATACAAGAAGAGCTTTAATGATGACTTCAGATCCCA | G | K | G | K | GYSNP0844 |
| TGTCGGCAAAGGCGAAAATGTAGAGCCACAGTATATATA | | CAATGACATTAAAGCTAATTGATACGATTCAAAGACTT[G/T]G | | | | | |
| TCGTCTTCGGGCAACACTTGTGTAAAT_NODE_1_length_636_ | | AATTGGGTACTACTTCGAAGAAGAAATAAAGAATATGTTAGA | | | | | |
| cov_125.220123 | | AAAATTTACACAAGTGTTGCCCGAAGACGATATATAT | | | | | |
| RAD_kmer_0002925_1_TGCAGGTTAGGAAAATTCAGCTTTG | 158 | AATGACATTGGATCATCTCTTTCAAACTTTATTTTAACCGGCA | R | A | R | G | GYSNP0845 |
| CATACTCACCACGAAGCTTATAAGCCGCCCGATCATACGC | | GGAAAGGTGGCAACATCATAGATTCAAATCTTCACTCA[G/A]TA | | | | | |
| ATAGGCCGCGGCTTCAGCCGACTCAT_NODE_1_length_500_ | | TTCACCATTATCATTAACCTCTAACTCTATTCGTCAGCCACTG | | | | | |
| cov_178.082001 | | GGCTCATCTGTTTACCTGAAACAAAGAGACCAGCT | | | | | |
| RAD_kmer_0002925_1_TGCAGGTTAGGAAAATTCAGCTTTG | 283 | GGGCTCATCTGTTTACCTGAAACAAAGAGACCAGCTCCAGAA | C | Y | Y | C | GYSNP0846 |
| CATACTCAGCACGAAGCTTATAAGCCGCCCGATCATACGC | | ATTCTCCGAACAACACATGGCGATATCGAACCCATTGA[C/T]C | | | | | |
| ATAGGCCGCGGCTTCAGCCGACTCAT_NODE_1_length_500_ | | AAAACCGCATTTCGGTTTGATCGGGTTTCAGATTATTACAAC | | | | | |
| cov_178.082001 | | AATCCTTACAAGAAAAAGCTTTACAGAGGAGTGAGAC | | | | | |
| RAD_kmer_0002927_1_TGCAGGTGTTAATATATTTGTTGAT | 218 | TGTATAAGATTTACGCATCCAATTTAATCGAAAGCAGTGACG | R | A | A | R | GYSNP0847 |
| CTATGTCTTATATTCTTGTACACTTCCCTAGCTCCATGCTA | | CAGTTCGTTATTGTCCTCTTCAAATACGAAAATGTATT[A/G]A | | | | | |
| CTGCTATTATAATAAAATAACCACC_NODE_1_length_594_cov_ | | TTCTGCATTCTTAAAAATATCAGTTTAGTAGTTTGGATATCTA | | | | | |
| 131.851852 | | TCTGAAGAATTTTGCTATGTTGCAGGCCCTTAAGGA | | | | | |
| RAD_kmer_0002928_1_TGCAGGGAAGATAGATAATATGTTA | 159 | CCATGGTTCTAAAAACTAAGAAAAGAAAACAGTTTTGAAAA | C | C | T | C | GYSNP0848 |
| GTTCACTTGAATACTGCATAAATATATGTACAGCGTGAAA | | ACAATTGTGATAAGTTAACATAATTTCAGAACTTACTGA[T/C] | | | | | |
| AAGCATGCACATTGGATTCTTGCTGC_NODE_1_length_494_ | | GATTATTAAGTTTTGGTATTTTACCATTTTAGGGTCAAATGTT | | | | | |
| cov_98.402832 | | GACACTGGTCGAGTATGCTCTATTGTTATACCGTGCA | | | | | |
| RAD_kmer_0002929_1_TGCAGGACTACGGTCCGCTGCTACC | 96 | GTTTCAGGTGCAGCATGATTTCTTTTGATTATCAAACTTAGGC | T | C | Y | C | GYSNP0849 |
| GGCTGCCGCCGCCCTGCCACATCGGATGCCTCTTGCGTGA | | GTCGGACCTAGTGCCTCATAATAATAAACATTTGAAC[T/C]TA | | | | | |
| TTACGGGATCATTTAAAATTGCGAAC_NODE_1_length_527_ | | TGTTATGTTTTGGTTTGTTATGTTATGTTGGTTATTTGAGACA | | | | | |
| cov_53.182163 | | ATTTGTAATTGTAAGATGATGAAATACATGATTGA | | | | | |
| RAD_kmer_0002929_1_TGCAGGACTACGGTCCGCTGCTACC | 466 | GGTCCTTGACTTGTAAGCCCAAACTTATTTCAAAATTCCTTAA | Y | T | C | T | GYSNP0850 |
| GGCTGCCGCCGCCCTGCCACATCGGATGCCTCTTGCGTGA | | GCCAGTAATCTTCAATCCTTAATCTTTAATGACAGTT[C/T]GC | | | | | |
| TTACGGGATCATTTAAAATTGCGAAC_NODE_1_length_527_ | | AATTTTAAATGATCCCGTAATCACGCAAGAGGCATCCGATGT | | | | | |
| cov_53.182163 | | GGCAGGGCGGCGGCAGCCGGTAGCAGCGGACCGTAG | | | | | |
| RAD_kmer_0002937_1_TGCAGGAGGAAAGTTTGTCTAGACA | 116 | TATATACATCTGCATATTTTCTTCTTTGAATTCGACTTGTTTTA | R | A | R | A | GYSNP0851 |
| AGTACATGGTGACGGTACCAAGGTTTATGGCTAAGTAACA | | AAAGACTTTGTGAATGATTGGTTTTAATCCATGAGT[A/G]TTA | | | | | |
| TGAGGGGTAAAAATGGTATTTACAGC_NODE_5_length_298_ | | TAAGGAATTAAGTTCACTAAATTAACCTAAATACACATGTTT | | | | | |
| cov_36.161076 | | TATGATTTCTGCGTAATCACAACTCAAATACTATA | | | | | |
| RAD_kmer_0002937_1_TGCAGGAGGAAAGTTTGTCTAGACA | 236 | ATGTTTTATGATTTCTGCGTAATCACAACTCAAATACTATATG | R | A | R | A | GYSNP0852 |
| AGTACATGGTGACGGTACCAAGGTTTATGGCTAAGTAACA | | AAGGGGGTTACACTTAGATGCTAGCCCAACTGGTTGT[G/A]TC | | | | | |
| TGAGGGGTAAAAATGGTATTTACAGC_NODE_5_length_298_ | | TATTGCCTTTATACTAGGGGCGGGCCGGGTGTTCGAAACCTG | | | | | |
| cov_36.161076 | | TGTCCTGTCTCCAACACGCAGCCTATTATTTTCTTT | | | | | |
| RAD_kmer_0002942_1_TGCAGGCACTTGTGGCCACAAAACG | 161 | CGGAGGTTTTATGCATTAAAAGTACACTTTGGGCGTTTTCAA | T | A | A | A | GYSNP0853 |
| ACAAATTTGGATCAGTTGGATTAATAGGATTGTCATCCAT | | ACCCTTCTGACGGGTTTGCTGTTACCTATTTGAACCTT[A/T]AT | | | | | |
| CTCCATGTTGCATGATTCATGATCTG_NODE_3_length_414_ | | AGATAAAATATAACCTTTATTGATCCGTTCATGGGTAAACAG | | | | | |
| cov_33.219807 | | GTTGAGTTGCCACCTCCTAACTGTTGTGAATGAAAC | | | | | |
| RAD_kmer_0002943_1_TGCAGGTAGTAAAATGGGCGGGTTG | 205 | ATCTAACTCAACGGTTTTCCCCGATTTTAGTAGTTTTAATTAT | C | A | M | — | GYSNP0854 |
| GGTAAAGGAAAATTTTTGGTACACTTTCCGTCTAAGGAAC | | ATTCCTCTACTGTAAAAACGCGCGAATCTTTGATTTA[A/C]AT | | | | | |
| TATAAATAACCAGTTTAAGTTATTTC_NODE_1_length_577_ | | GTATGTTGTTCTTAGGTAGTCAATTTCAATTTTTCTAAAATTT | | | | | |
| cov_113.956673 | | GTCTAAACTCGGAACTCATAAGTAATGGACAAAAG | | | | | |
| RAD_kmer_0002949_1_TGCAGGGACTTATCCTTTAATAACT | 132 | CTTCGGATTGCCTCACGCTTTTTTAAGCCAATGTTACAAACCA | C | G | G | G | GYSNP0855 |
| TAAGTGGCCCGGTTCCGAGGTTTCCTTCTAAGACCTTCAA | | AACACGATTTACAAACCTTTGACATCAAAGAATGGTT[G/C]TT | | | | | |
| GTAAGCTTTTAATCACTCCCCAATCT_NODE_1_length_577_ | | GATTATGCCTTCGTCTCCACCAAATCAGGGCGAGTCCAAATA | | | | | |
| cov_121.298096 | | TGAGCAAAGAGATGCACCCGAGGCTTGTACTGATGG | | | | | |
| RAD_kmer_0002965_1_TGCAGGTACTTCATTCTCTCACTAAT | 232 | GGTTGTTTCTGATAAGGAAAAGAAAATAAAAGTTGGTTCTGA | A | A | G | G | GYSNP0856 |
| TTTCTCCCTCCTCACCTGTTTACGGTCACAATCATTTATCT | | GGGTGATTCAAGGATCACACAACAAAATAGCAACAGCA[G/A] | | | | | |
| TTTTGCAAAACAAATAATTATATA_NODE_1_length_462_cov_ | | AGAAGGCTCTACCAATACTTCATCTAAGGACAAGTCTAAGGT | | | | | |
| 91.774895 | | TTCCGAGGTTCAAAAACCGGATTATATTCATGTTCGAG | | | | | |
| RAD_kmer_0002969_1_TGCAGGACTTCCCCTTCTGATGAAT | 155 | TGTCAATCTATCTCTCCCTCTCTTACGTTATATCTGTAAACAA | A | T | T | W | GYSNP0857 |
| TTTCCATCTTTATAAATGTATAACATTGGCACCCCAGTAG | | GAGTTTTAGGTGAGCAATAACAAAAAATAATTATTGC[T/A]GT | | | | | |
| ACAGCTCTAAGTTGATTACCTTTTAG_NODE_1_length_653_ | | TATGTTCTCTCAGGTATGGTGACGTTCAAGGTCTTAACAAGCA | | | | | |
| cov_118.039818 | | AGCAACTGCTGAAGTATTCGGTAAGGAGCAAGTTT | | | | | |
| RAD_kmer_0002969_1_TGCAGGACTTCCCCTTCTGATGAAT | 332 | TTATGAAGTCCGCCCCCCGAATGGTGAAAGTTTAGAAATGTG | T | W | T | W | GYSNP0858 |
| TTTCCATCTTTATAAATGTATAACATTGGCACCCCAGTAG | | CTTGCAAAGAGCCGTTACCTACTTTAAAGACAATGTAA[T/A]A | | | | | |
| ACAGCTCTAAGTTGATTACCTTTTAG_NODE_1_length_653_ | | TGACCTTTTTTTGTATATTCAAGTTGAAAAGAATCTTTAGAAT | | | | | |
| cov_118.039818 | | CGCTTAGCGTAATAGAAAATGGTGCTGACAATGTTT | | | | | |
| RAD_kmer_0002973_1_TGCAGGGAACAAACCGATAAGCT | 459 | CCAGCATCTTCGGATGGCATTGCTGTGAGTCAACTCGATAGC | C | M | C | M | GYSNP0859 |
| TCGAAGTATAAGAGGTGAAAGTGAAAACTATAACGCCTT | | GGGAATCCGTATGTAATGGATATAAGTGGCGTAAGGGG[C/A] | | | | | |
| GTGCACCTGTTTTCTAGCTCGTGACCTT_NODE_1_length_610_ | | GGGAGGAACCGGATAATCGACGGTCCTGTGGAGAAAGTTGC | | | | | |
| cov_126.324593 | | GGAGAGGCGACAACGAAGGATGATAAAAAACCGCGAGTC | | | | | |
| RAD_kmer_0002981_1_TGCAGGAGTACGTTTTGCATATCAA | 418 | AAATAATGCGATAGTTGTCATTAAGTCAAAATTAGATACTGA | M | C | M | C | GYSNP0860 |
| CTAACCAAAATTCTCTAACTAGTAAAAATAGATAAAAAA | | GTACCCAGTTAAAGGTAAATCAAACTGTTGTTAAATGT[A/C]A | | | | | |
| GAGAAAAGAAGAGAAAAGGTAACATTC_NODE_1_length_674_ | | ATCATGATGGTTTTTTATCCGGTTTTAACTTCACTGTGGATGC | | | | | |
| cov_99.703262 | | ATACTTTACAACAAAGCTAACTTCTTGCTGTGCTGG | | | | | |
| RAD_kmer_0002987_1_TGCAGGTAGGAATAACCTCTTCTTC | 260 | GAAGCGGCTTGATGTTGATTACATTGATCTTTACTATGTTCAT | A | G | A | G | GYSNP0861 |
| TGCATCTCTAGTCCACAAAGACCACTCCAGTTGTACTGCT | | CGCATCGATACAAAAGTCCCTATTGAAATCACAGTTA[A/G]TT | | | | | |
| GTTATTGGATGAATCGCGTGTGCTCT_NODE_1_length_565_ | | TCACTTCTTTTTTATTGGAGCTTATTTAATAAATGTTTGTTCAT | | | | | |
| cov_129.366379 | | GAGCCGAGTTTGAGCTTTGGTAACATAAACAAGC | | | | | |
| RAD_kmer_0002987_1_TGCAGGTAGGAATAACCTCTTCTTC | 425 | CTTGGCTTGGCTTGGCTCGGCTCATTTACAGATATAATATTGA | A | W | W | A | GYSNP0862 |
| TGCATCTCTAGTCCACAAAGACCACTCCAGTTGTACTGCT | | TGCCTGATTTTAAGGAAGAAATTGTTTTGTAGATGGG[A/T]GA | | | | | |
| GTTATTGGATGAATCGCGTGTGCTCT_NODE_1_length_565_ | | ACTGAAGAAACTGGTTGAAGAGGGTAAAATAAAATATATTG | | | | | |
| cov_129.366379 | | GTCTATCTGAGGCTGGTCCAACGACTATTAGAAGAGC | | | | | |
| RAD_kmer_0002988_1_TGCAGGTTCATACAACCGCTACTAG | 105 | TTGGGAACTTTATCGAATCAAAACTCAAAGCTGAAATCTTTTT | A | A | A | T | GYSNP0863 |
| ACAAACAAGAAACACCAGAATCCGTAATGCAGCTCTTTG | | CACTTATAAAGTGTTTGGGAATCAAAGATATAGACCA[A/T]G | | | | | |
| AAACATCAAGATCTCTGAGAAATAAGC_NODE_1_length_510_ | | AACCGGTTGACTTCACTGAAAGTCAATCTTTACGATCATTGA | | | | | |
| cov_91.082352 | | CGATCAAGAACTGCATTGGATTCGGAAACTCTAATTT | | | | | |
| RAD_kmer_0002991_1_TGCAGGTCTGAATTATGCTCTCTCT | 172 | ATTTTAGTTGTTCTATAAGTTAATATTATGTAATCAAATGAAA | R | A | R | A | GYSNP0864 |
| ATCTCTCTCTTTTTTTTCTTTCAATATTGGATGCGGGAAAA | | GGTATTTAAAATTACCTGAGGCAGATAGTGTACCCCA[G/A]AT | | | | | |
| ATGTGCGAGTTGGGTAATAGAATGG_NODE_1_length_554_cov_ | | TTTGTCAAGATATTTAAAAATCTCAAAAGATGCTTCCTCAAC | | | | | |
| 154.027069 | | AGATATCAAACCTGACCCATTAGGCCAATGAGCCAG | | | | | |
| RAD_kmer_0002992_1_TGCAGGAGATGTGCTTGTTTGACCC | 344 | GTTAAATAATCGAAACAGGTTATTCCTTCTTGGTTTGGAAAA | W | W | T | A | GYSNP0865 |
| GTGATTGCAGCTTGAGTTACTGACATGTCACCGTTGTTGA | | ATACGGTAAAGGTGATTGGCGAAGTATATAAGGAATT[T/A]T | | | | | |
| CACTTGTTATGTCATGAATGCTTGAT_NODE_1_length_496_ | | GTTGTGACAAGAACACCAACTCAAGTTGCAAGCCATGCTCAG | | | | | |
| cov_144.965729 | | AAGTACTTCATTCGCTTGAATTCAATGAATAGAGACA | | | | | |
| RAD_kmer_0003000_1_TGCAGGACACCAGGAAAATCAAAA | 332 | TTTTTGGCAGAGTATTATAATTGGAAAATGGTGTTTCAAATCT | C | — | C | T | GYSNP0866 |
| TCCAGATTATGATAATAGATTTAAAACATCTTACAAAATT | | TCTAATATATCAACGAGTTTAAGTAATTAGTAAAATT[C/T]TT | | | | | |
| CTAATAAAAATTTAAAGGTTAGTATCA_NODE_1_length_563_ | | TTTATAGGCTGAATGAATATCTTAGGGATATTATACTGTTGGT | | | | | |
| cov_53.239788 | | AGGAGAACTGTGTGTGAATCTTGAAAGGGCACTTT | | | | | |
| RAD_kmer_0003004_1_TGCAGGAACCAAACTGAAACTGAA | 481 | CTCTCATAACTTAATCAAGCTCGAGCTTGAGCTCGAGCCTGG | C | C | C | T | GYSNP0867 |
| AGCTGCAATATTTTGGCAAAAAGTTCTCCAAAATACCATA | | TCGAGCTCGGCTCGACTCATTTCACCCTAGAAGAAAGT[C/T]A | | | | | |
| CCAAACAAATAAATAAAGTACTAAAAT_NODE_1_length_583_ | | AAGTTAAAGAAGTGTTCCACAAAAGGGTTAACAATTATTTTA | | | | | |
| cov_93.418526 | | GTACTTTATTTATTTGTTTGGTATGGTATTTTGGAGA | | | | | |
| RAD_kmer_0003006_1_TGCAGGTAGGGTCCGAACCAGGT | 189 | TAATAAATCCGTTTGGACCAATTTGGAGGCGATTAGGGCTGC | T | G | G | G | GYSNP0868 |
| ATTTTGTGCATTTCTGTATACAACCTTGGGTGCTTTCCAAT | | CCGGAACTCTCCAGAACAGCCCCTAAATTCTGGACAGC[G/T]T | | | | | |
| TAAAGCCTACATGACCTATTTATACT_NODE_1_length_543_ | | AGACGCGTGTGGAAGGTTCAAAATCAGCCCAACGTGACCCTG | | | | | |
| cov_150.727448 | | GGACACCCTAAAACGGCTGCACAAATCAGCCCCAATG | | | | | |
| RAD_kmer_0003008_1_TGCAGGAGATTCAAGGAGCTCTACA | 510 | AAACGATTTAGATACGCGTAGAAACACAATAATTTTAAGCTC | C | T | C | Y | GYSNP0869 |
| AGTGAGAAATTGAACTTTCGGATTTCAATCCAGGTTTTGT | | TGCAGAAATCAAATATCTTGACAAATCTAAAGCTCCAA[C/T]T | | | | | |
| AGTTTTAAACCTTTCAATAAATTTCA_NODE_1_length_602_ | | TGTTAAAAAGAGAGTCGAATGCAACATGAAATTTATTGAAAG | | | | | |
| cov_76.803986 | | GTTTAAAACTACAAAACCTGGATTGAAATCCGAAAGT | | | | | |
| RAD_kmer_0003009_1_TGCAGGCTGAGAAAGCCAAGGTGA | 143 | ACATGTAAATCATATATTCTTACCATGGGAAAAGCAAGATAA | Y | Y | Y | T | GYSNP0870 |
| TCCAAACACTGCTTTTTCTTTCAGGATTCAACACATTAATG | | TATAGTCCAAGTCCAGTAAATGTAACAAGAGGAACCAA[T/C] | | | | | |
| CAAACCATGTTTGGTACTAGATTACC_NODE_5_length_394_ | | GAAAGAGGGCTAAGATAGCTATACATGATTCAAGGTTGCAAA | | | | | |
| cov_31.497461 | | AATATAACTAAAAATAAAAAAATAATAAAAAAATAAAA | | | | | |
| RAD_kmer_0003009_1_TGCAGGCTGAGAAAGCCAAGGTGA | 303 | ATAAAATGAATTTGATCAAATCAAACACTTTACTTGACAATG | M | M | M | A | GYSNP0871 |
| TCCAAACACTGCTTTTTCTTTCAGGATTCAACACATTAATG | | TTTCTCCACACGCCAAGAAATCCAAGAGTTGTCTGTAA[A/C]G | | | | | |
| CAAACCATGTTTGGTACTAGATTACC_NODE_5_length_394_ | | CGGATGCCACGACAAGAGCACCTTGTATGCCTCTCATCGTTT | | | | | |
| cov_31.497461 | | GTGCAAATCTCTTAAAAGTACCCGCGTCAAACCATAT | | | | | |
| RAD_kmer_0003015_1_TGCAGGTTAGAAGGTATATTTCTGT | 369 | ACCGCTGTGTCAAAGCTATGGCCGACAAAACTTAAACTATGC | A | G | A | G | GYSNP0872 |
| CGGTGACCGCTGAAAAGTTTAAAGATAAGATTGTGGTCA | | ATTTAACATCACACCGGCCTAAATGCGTATACAAACAA[A/G] | | | | | |
| AGATTTCTTGCCATGTTTTACCGTTGT_NODE_1_length_641_ | | CCAACCAATGTTATGTATCATGGTAAATGCCAATGGAATGGC | | | | | |
| cov_148.524185 | | CGCTTTCAACAAAATTTATAACACCACGTGAAACCGTG | | | | | |
| RAD_kmer_0003018_1_TGCAGGATTTGAAGCCCCCACCTGT | 275 | GAGTCGATGGGGTTTCATATGCAGAATGTTCCCTCACTTAATC | A | T | A | W | GYSNP0873 |
| TAGAGATAGAGAGATGAAAGCCATGTAAATAGTATTAAG | | GTCTCATGCTTACTGAAGCCAAGGTAAAAGTAAAGTT[A/T]AT | | | | | |
| CTTTATATTATTTATGACGACATCCAA_NODE_1_length_547_ | | TATTTTATCATAATGATCAGTTTTCACTTACTTTTTCTTTTTCT | | | | | |
| cov_105.170021 | | ACTTTTTTTTACAATAATGTTTTTCTTTTAAATA | | | | | |
| RAD_kmer_0003018_1_TGCAGGATTTGAAGCCCCCACCTGT | 361 | TTTATCATAATGATCAGTTTTCACTTACTTTTTCTTTTTCTACT | A | T | A | W | GYSNP0874 |
| TAGAGATAGAGAGATGAAAGCCATGTAAATAGTATTAAG | | TTTTTTTACAATAATGTTTTTCTTTTAAATATTTGC[A/T]GACG | | | | | |
| CTTTATATTATTTATGACGACATCCAA_NODE_1_length_547_ | | GACAAAATACAAGTGCTTTTTAAATTTATATATATTTCAATTT | | | | | |
| cov_105.170021 | | TTATTGTAATGATTGTAACCAATGTTTTTATAA | | | | | |
| RAD_kmer_0003018_1_TGCAGGATTTGAAGCCCCCACCTGT | 492 | TTGTAATGATTGTAACCAATGTTTTTATAAACTGACCGGATGA | C | — | G | G | GYSNP0875 |
| TAGAGATAGAGAGATGAAAGCCATGTAAATAGTATTAAG | | TAAACATGGTGTTTTACTGATTTAAGAATTGGATGTC[G/C]TC | | | | | |
| CTTTATATTATTTATGACGACATCCAA_NODE_1_length_547_ | | ATAAATAATATAAAGCTTAATACTATTTACATGGCTTTCATCT | | | | | |
| cov_105.170021 | | CTCTATCTCTAACAGGTGGGGGCTTCAAATCCTGC | | | | | |
| RAD_kmer_0003019_1_TGCAGGGTAGAGGTAAGTTTGTCTA | 148 | GTTATTGGCATTAAATCCTCATTTTTCTCGAGCATTTACTCTCT | A | G | G | G | GYSNP0876 |
| CATCTTACCCTCCTCAGACCCTACCTTAGCTTTGCTATTGG | | TGGATACTGTAGCTCTCATTTACTATGCGGTGAGCC[G/A]GGT | | | | | |
| TGGGATTTACTGAGTATGATGATGA_NODE_1_length_523_cov_ | | AAATGGGTAACATCTCTGTAGTGAGGCTAACAGTTGCTTTGT | | | | | |
| 76.814529 | | ATTGCACAACTATTCTAAAGTCCCGCCTAATCGCC | | | | | |
| RAD_kmer_0003019_1_TGCAGGGTAGAGGTAAGTTTGTCTA | 224 | AGCCGGGTAAATGGGTAACATCTCTGTAGTGAGGCTAACAGT | C | T | T | T | GYSNP0877 |
| CATCTTACCCTCCTCAGACCCTACCTTAGCTTTGCTATTGG | | TGCTTTGTATTGCACAACTATTCTAAAGTCCCGCCTAA[T/C]C | | | | | |
| TGGGATTTACTGAGTATGATGATGA_NODE_1_length_523_cov_ | | GCCCACCAAACACAAACACAAGCACATTGTTGCAACATATAC | | | | | |
| 76.814529 | | ATATTGCTACTCTTGAGAGGAGAAGAATTAAGGTAGC | | | | | |
| RAD_kmer_0003019_1_TGCAGGGTAGAGGTAAGTTTGTCTA | 415 | AGCTTATTGTTTAACCCCCCAAAATAGTCCTGGTTACATCATC | G | R | R | G | GYSNP0878 |
| CATCTTACCCTCCTCAGACCCTACCTTAGCTTTGCTATTGG | | AACTACACCTAATGCCATGGCTCTAATTCAATCATTT[A/G]CA | | | | | |
| TGGGATTTACTGAGTATGATGATGA_NODE_1_length_523_cov_ | | GCCCTTCGTTTTCTCAGCATCAACTAGACCTAATCTCATCATC | | | | | |
| 76.814529 | | ATCATCATACTCAGTAAATCCCACCAATAGCAAAG | | | | | |
| RAD_kmer_0003020_1_TGCAGGTTAACAATGCAAGACATGT | 206 | CAGTTTCCATGACAGGTGATCCTTCTTTCTTTTTTCTTTCTAGT | A | R | R | G | GYSNP0879 |
| TGGGGACATCAATTCAGGAGATTCATCCTTCCAAAAGGTT | | TTTATAATAGTGTTATATATCTCGCAATCTCGACCC[A/G]CTTA | | | | | |
| GTTAATACTTTTTGAACTGGTGAGGT_NODE_3_length_365_ | | CTTAAAAGGGCTAATCAGGTATTATTTTATTTTTTTGATGGGT | | | | | |
| cov_50.430138 | | CAAACAGTTCGATAGCTGCCCAAAATGTATTTT | | | | | |
| RAD_kmer_0003020_1_TGCAGGTTAACAATGCAAGACATGT | 311 | ATTATTTTATTTTTTTGATGGGTCAAACAGTTCGATAGCTGCC | Y | Y | T | Y | GYSNP0880 |
| TGGGGACATCAATTCAGGAGATTCATCCTTCCAAAAGGTT | | CAAAATGTATTTTTAGAGCAAAAACCATACTAAATTG[T/C]GA | | | | | |
| GTTAATACTTTTTGAACTGGTGAGGT_NODE_3_length_365_ | | TATTGAATAAAGTTTATTATTTTGATAATTTATATATATTTTTA | | | | | |
| cov_50.430138 | | ATGTATATATGTTGTTGATTTATTGTGATAATGT | | | | | |
| RAD_kmer_0003023_1_TGCAGGTTTTGTAACGGGGAAGAAA | 130 | CGTAGAATTGCCTTTTTTCTTCTTTTCGTTAATCATTCAGTTG | C | — | Y | T | GYSNP0881 |
| AGAAGACGTTGTGGAGGGGCAGCCGGACAGTGGTGACTG | | TTGCCAACGGTTTCTTTGAGGTCGGAATCGAAACGT[T/C]TGG | | | | | |
| GTAGTAGCGGAGGGTGGCTACGAGGCG_NODE_1_length_494_ | | GTTATCACAACGATGGAACGGTATGATAGCCGGTATCATGAA | | | | | |
| cov_64.419029 | | ATCGTTCACATTAACAAAATTCGTTTGGACCAGCG | | | | | |
| RAD_kmer_0003035_1_TGCAGGAGCCGGAGCCCTCTCATCA | 88 | TGTGATTGTTTTTCTGCGCTGCTTAAAGCATCCAGAAAATCTG | R | A | A | — | GYSNP0882 |
| ACACTCCGATCAGATGCTCTGCACTTACCAATGCCGCCAA | | CTTTGACCTGTAAGAACAATAAGTATTTCACTACAAC[A/G]TG | | | | | |
| CTCGTAATCCTCTCTCTCTCTCTCTA_NODE_1_length_617_cov_ | | AAAAGTAGAAAAGTAAGAAAATAGCAAATGACTAAGCCTCA | | | | | |
| 116.458672 | | CATACAATTTCACACTTCATTACTAAGTCTTATTGTC | | | | | |
| RAD_kmer_0003040_1_TGCAGGGAGGAATGCTAGGTACAG | 159 | TTATTCAATAGAACGATTACACTGAGAAATCAAAAAACTAAC | A | R | R | R | GYSNP0883 |
| TTAATCTTAGAAATAATATGACGCTGTGGCATATACTGGG | | AAAGAAGACGGGATGGCTAAACAGCTTATTCGAGAAAG[A/G] | | | | | |
| CGAGCATGTTAAAAAAGGTCGAATATA_NODE_1_length_537_ | | TGCTGAGCTTTCTCTCTCCCAAAAAGCTGCTCAACGAGTGCA | | | | | |
| cov_107.378029 | | AGAGCGTATTCCATGGTGGTTCCCAAGCCTTGACTAGT | | | | | |
| RAD_kmer_0003041_1_TGCAGGTTTACAGTACGATAATTCG | 272 | TTATTCCTCCCAATTTCGCCATTTCTATAACTATTTCTAAACCC | T | T | Y | Y | GYSNP0884 |
| GGCTTTTGGTAAGGATAAAAGGGTGGCTGCAGCTGTTGCT | | AACATAAAACTCAAGAGCCCCAAACCCGTCTTCCAT[T/C]CTT | | | | | |
| GTTGTAGAATGGTTGAAGCTGAAGAG_NODE_1_length_598_ | | CTGTACGCCAACAATGCTGTCGAATACGAAGCTGGAGACGGA | | | | | |
| cov_151.672241 | | GACAACCCCTTCGTTTTAATCTCATTAAAAACTTC | | | | | |
| RAD_kmer_0003041_1_TGCAGGTTTACAGTACGATAATTCG | 373 | TGTCGAATACGAAGCTGGAGACGGAGACAACCCCTTCGTTTT | C | T | C | Y | GYSNP0885 |
| GGCTTTTGGTAAGGATAAAAGGGTGGCTGCAGCTGTTGCT | | AATCTCATTAAAAACTTCTAACGCTTTTGTTTCGTCCC[C/T]TT | | | | | |
| GTTGTAGAATGGTTGAAGCTGAAGAG_NODE_1_length_598_ | | GAGCTAACGAAATCCCCATCAATGTGTTGTAAGTCACCACAT | | | | | |
| cov_151.672241 | | TTGGAATCACACCACTGCTAACCATATCATTGATAA | | | | | |
| RAD_kmer_0003041_1_TGCAGGTTTACAGTACGATAATTCG | 525 | CATTGATAACCTTCTCCACATGATCAAACTCTTCCGCTTGTT | G | A | G | R | GYSNP0886 |
| GGCTTTTGGTAAGGATAAAAGGGTGGCTGCAGCTGTTGCT | | AATCGCACCCAAAAGACTATTATATATAAACAAATTC[G/A]G | | | | | |
| GTTGTAGAATGGTTGAAGCTGAAGAG_NODE_1_length_598_ | | CCCCGGGCTCTTCAGCTTCAACCATTCTACAACAGCAACAGC | | | | | |
| cov_151.672241 | | TGCAGCCACCCTTTTATCCTTACCAAAAGCCCGAATT | | | | | |
| RAD_kmer_0003045_1_TGCAGGGATGGTTTTTATTTAATTA | 381 | GAGGTTTATTGTTAAGGTAGTGAAAGAGCTGTTATGCAAAAA | C | C | Y | Y | GYSNP0887 |
| CTTTTTGGAAAACAAAAACGAAATATAAAAATTTATTAGG | | GAAAAAATGAAAATTGATATGTTTTATGCAATTAGTTT[C/T]T | | | | | |
| TTATATCCTAGGGGTTTCACAGGATA_NODE_1_length_568_ | | CAATAATATTATTAAATTATTAAAATACAATATTATTAAGTTA | | | | | |
| cov_109-846832 | | TTAATAGATAATCTTTATAACCCATTTATTCTATAA | | | | | |
| RAD_kmer_0003050_1_TGCAGGCGAAACCACCGTTAAAAC | 212 | CAGATATCATGATAAAATAGTTGAGGTGCTATAGGTGTATAA | R | G | R | G | GYSNP0888 |
| ATGACAACACTGAGGGATTGTAAGCTTTGGTGACCGTCCA | | CTATGAAATAGAGAGGCTAAAGCCCATGGTGTGAAAAA[G/A] | | | | | |
| GGCGGGGAAAGGTTTGAGCGATAAGAT_NODE_1_length_352_ | | GCGGGAACTATATATTAAAGCATATTTGCTTAGCAGCCACAA | | | | | |
| 52_cov_205.315338 | | ACCAAACCCTAGCGGCCACCCACCATGAACACCATCTT | | | | | |
| RAD_kmer_0003052_1_TGCAGGCGGAGAACCTCCACCACAT | 393 | TTCATATTCTACACCAACATATGGTATTCATGTGTATCCGATT | — | A | A | W | GYSNP0889 |
| TGATCGTCTATTCAGTAAGCGTCATTATTCATTCGATGTTC | | TATGTCTGAAGACGAGCAGGTCTATACGTACATAACT[A/T]TA | | | | | |
| TCTCGTTTAGTAGCTCGACAATTTG_NODE_1_length_594_cov_ | | AGTATGAAAAATTTTAGATAGCTTACTTGTATTCGTATTAGAC | | | | | |
| 90.597641 | | TATTAGTAGTCAATGCAAGAGAACCCAACAGTGGC | | | | | |
| RAD_kmer_0003053_1_TGCAGGGATGGCTAACATGTCAGCC | 100 | CATTCTTTTAGTTAATATAAAAAAATGATTAATTAACTAGTTT | Y | Y | Y | Y | GYSNP0890 |
| ACCGGGCTACTACAAAAAGCAGCTCAAATGGGGTCCACA | | CTTAAATAAGTAAGGTTCGTATTTGATTTCACGTGTA[C/T]AC | | | | | |
| AGAAGCACAAACTCTAGGGTTTCCGGT_NODE_1_length_408_ | | AAATATAGAATTTATAGCATGTGAAATATACGTGTAACAGTC | | | | | |
| cov_39.399509 | | ATCACAAAAAGAAAAAAGTTGTTAAAACTTACAACT | | | | | |
| RAD_kmer_0003059_1_TGCAGGATCGAGTATCGACACTGAA | 150 | AACATGACAGTCTTGGTTACATAAAAGTCAATGCCATAAAAT | T | W | W | W | GYSNP0891 |
| ACTCCACCTAATGATGGAGAAAACGCAACACCTAAGCTC | | ATATATCTATATGTGTATGTGTTAACATTAAAACGATA[A/T]A | | | | | |
| CAAGAAAACGGTAATTTATATTCTTAC_NODE_2_length_574_ | | AAATTCGCAATTTTTCTGGTCGCCAACTTAAGATGAGTGAAT | | | | | |
| cov_46.202091 | | AATCAATATATAATAAAAAAAATATGCTGAAATTTGA | | | | | |
| RAD_kmer_0003061_1_TGGAGGTTAAACGAAGAGATTTTCT | 343 | TTGTGGTGGTCGGCAGTGGTTGTAGATGATAAAGGGGCGGTG | K | G | K | — | GYSNP0892 |
| TTAGGAACGATGGTGTTGCATGTGGTGGGTGGAGACGAT | | GCTGTTTATACTGACAGTGATTTTGTTGAGTGGTAGTG[G/T]A | | | | | |
| GAGATTTATGGTTTGCGATGGTCGTGA_NODE_1_length_555_ | | GGCAGGTAGGGGTGGTGGTAGCAGGTTGGGTCGATGGTAGC | | | | | |
| cov_88.661263 | | AAAGGTAAATAAAGAGAGAGAGAGAGAGAGAGAGAGAG | | | | | |
| RAD_kmer_0003061_1_TGCAGGTTAAACGAAGAGATTTTCT | 397 | GACAGTGATTTTGTTGAGTGGTAGTGGAGGCAGGTAGGGGTG | A | G | R | — | GYSNP0893 |
| TTAGGAACGATGGTGTTGCATGTGGTGGGTGGAGACGAT | | GTGGTAGCAGGTTGGGTCGATGGTAGCAAAGGTAAATA[A/G] | | | | | |
| GAGATTTATGGTTTGCGATGGTCGTGA_NODE_1_length_555_ | | AGAGAGAGAGAGAGAGAGAGAGAGAGCCATGGTTCCCACCG | | | | | |
| cov_88.661263 | | CTCACCAGCCCAGCTACCCATCTCTGCCGCCATTCCCCT | | | | | |
| RAD_kmer_0003064_1_TGCAGGATAGAGTGAAGTGTTTGGG | 132 | TTGCCGGGTGTCACACCACATCAAACAACATTTAATACTTTG | — | A | G | G | GYSNP0894 |
| GTTTTGTTTCAGGGTTTCTGGTTTTACTTTGTCTACAATTT | | ATTCATTCCCACATGGCAACCAAACAAGACATGGAATG[G/A] | | | | | |
| ACTATTATTATTTTTATCTACTTT_NODE_1_length_595_cov_ | | TAATGATTCATTCCATTACCTCGTCCATTCCAATCCTTCATCC | | | | | |
| 72.547897 | | ATTCCATTACCCCATACCAAACAGACCCTAAGCTAAT | | | | | |
| RAD_kmer_0003064_1_TGCAGGATAGAGTGAAGTGTTTGGG | 199 | AAGACATGGAATGGTAATGATTCATTCCATTACCTCGTCCATT | — | T | G | G | GYSNP0895 |
| GTTTTGTTTCAGGGTTTCTGGTTTTACTTTGTCTACAATTT | | CCAATCCTTCATCCATTCCATTACCCCATACCAAACA[G/T]AC | | | | | |
| ACTATTATTATTTTTATCTTACTTT_NODE_1_length_595_cov_ | | CCTAAGCTAATAGATACATAATGACTAGAAATCATCCTTTTC | | | | | |
| 72.547897 | | ACTCCACATCAAACCATCATATCTATACCCCATTTT | | | | | |
| RAD_kmer_0003065_1_TGCAGGGGCTGGTCAGGTTACTAAA | 118 | TCACTTTTCTCAGAGAAGCGTCTGAATTCGCGTACCCTGTAAA | C | C | M | M | GYSNP0896 |
| GAAAAACTTGAATTCAAGCATAGACTTTCAATAGCTCTTG | | AATATACAGGATCAGAATAATATTTCAGAGAAAAAAG[C/A]G | | | | | |
| GGGCAGCTAAAGGTGATCCTCTGCAA_NODE_1_length_575_ | | AATAAGAAAAAGAAAACCATGAAAAGAGTATAACTCTGGAG | | | | | |
| cov_110.361740 | | CAAGGAATATGTCATCAATTCTAATTTGAGAAGATGGG | | | | | |
| RAD_kmer_0003065_1_TGCAGGGGCTGGTCAGGTTACTAAA | 184 | TTCAGAGAAAAAAGCGAATAAGAAAAAGAAAACCATGAAAA | G | R | R | R | GYSNP0897 |
| GAAAAACTTGAATTCAAGCATAGACTTTCAATAGCTCTTG | | GAGTATAACTCTGGAGCAAGGAATATCTCATCAATTCTA[A/G] | | | | | |
| GGGCAGCTAAAGGTGATCCTCTGCAA_NODE_1_length_575_ | | TTTGAGAAGATGGGCCCGCTGAGTCAACTCGCCCCAAGAAAT | | | | | |
| cov_110.361740 | | TTCGGACACCTGCATCTGCAACTTTGGCTATGAAGTTC | | | | | |
| RAD_kmer_0003066_1_TGCAGGGAGACTTCGTCCTCAGGTT | 86 | CACTAAAAAACCCCAGTTACTATTTTGTTAAAAATTTCTTTCC | K | — | T | K | GYSNP0898 |
| CACGGACAAACCTTGAAGTACAACATGAAACTTAGAGAG | | AAAGTATATGCTATTTACAAAAAATAACGATGAGAAA[T/G]G | | | | | |
| GGTCGTGGGTTTTCTCTTGAGGAATTA_NODE_1_length_585_ | | AGAGTTTACCTTCATTTTCCGTGCTCTCCTTGGGAAAACAACC | | | | | |
| cov_125.111115 | | AATTTAGCCTTAAATGTTTTCAGCCTCGCACGTTA | | | | | |
| RAD_kmer_0003066_1_TGCAGGGAGACTTCGTCCTCAGGTT | 328 | CCTGCAGCCTGTGTAAAAAAACAAAAATAACATTTAGTGATT | R | — | G | G | GYSNP0899 |
| CACGGACAAACCTTGAAGTACAACATGAAACTTAGAGAG | | TGGGTTGTATCATACACAAATCAATAAAATAGTTTACC[G/A]T | | | | | |
| GGTCGTGGGTTTTCTCTTGAGGAATTA_NODE_1_length_585_ | | TGCCTGTATCACAGAAAACAATCTTCCTAACAATGATATCAT | | | | | |
| cov_125.111115 | | CTCATGTGTCTAATTAAATATAACATGAGTCCTATAC | | | | | |
| RAD_kmer_0003067_1_TGCAGGGTTAGTCAAAGATTAATTT | 153 | TTTGAACCAGCCTAAAAAAGTACTCTTTCTGAATTTTGACACA | C | T | T | Y | GYSNP0900 |
| ATGCTTCATTTATTATTTATCAATAGATGTTGATTATATTT | | ACCCAGTTTTGTCATGTAACACAACCCACATGAAATG[T/C]AA | | | | | |
| ATTTACTTTTATTTAAATTTTTTAT_NODE_1_length_603_cov_ | | CAGATGAAACCCTGATAAATCATTTTCTTCAAAAACTATATT | | | | | |
| 103.018242 | | ATTACTGAAATAATGTTTGTAATCATATGTCACCCT | | | | | |
| RAD_kmer_0003067_1_TGCAGGGTTAGTCAAAGATTAATTT | 392 | TCATGTGGGCCCAAACCGTTTTGACCCTTTACACAAACCCGC | C | T | T | T | GYSNP0901 |
| ATGCTTCATTTATTATTTATCAATAGATGTTGATTATATTT | | CTAACCAGCCCATCTTCCACATCAAATTATAAGTTAAT[T/C]G | | | | | |
| ATTTACTTTTATTTAAATTTTTTAT_NODE_1_length_603_cov_ | | AAAGGAGGGGCAATTAAGCATATAATGTTGCAGGTTTACCTT | | | | | |
| 103.018242 | | CATTCCAGGCATTTGGACCCACTCAATAGCAGTTCCA | | | | | |
| RAD_kmer_0003068_1_TGCAGGCAGTTTTGAACATCAACAA | 137 | TTGTCAATTGTTAAGATTTACTAAATTTATTAGGTTTTTTTATG | — | C | Y | C | GYSNP0902 |
| CGAGCTCTAGAACTTCAAAACCCTTATTCAAGATAACACC | | GACCCAGGACTTGAAATCAAGCAGAAGGTTGACTCA[T/C]CC | | | | | |
| AAACATAAGCATTGATTTAAACATTG_NODE_1_length_554_ | | CTTTATAATCACACGCTCGCCACCATTCTGGTGGAATATGCTT | | | | | |
| cov_53.424187 | | CTGCGGTAAGCTCACTCTGGTTTGTTATGCGCTAT | | | | | |
| RAD_kmer_0003070_1_TGCAGGGATATGAATGAATCGTGA | 192 | GTAAAATTAACTTGATTTTTATATTAAAACCTATTAGCCCAAT | G | G | R | R | GYSNP0903 |
| AAATCGTATTGTGTGTGTGTTTAGTTTAATTTAGACTTGTA | | TCTTGAAACAATCATTTGGATGCAGGGAAGGAATAAG[G/A]G | | | | | |
| CACGAACCGAACGGACACAAACGGG_NODE_2_length_393_ | | TATCTTTAAATGTTTATTTGACTATTTACTTAATTATGTCATTT | | | | | |
| cov_31.737913 | | TGTGCTGTACATTTGTGTTTGTTTATGCTCGTTCG | | | | | |
| RAD_kmer_0003079_1_TGCAGGAGAGGGTATTCGGACGTG | 297 | CAGATCACCCCTAAGCTAAAGTTATCAAGTAAATCACACACA | — | T | C | T | GYSNP0904 |
| GCACCACCGTGCCCCTCCCTGCGGCCCCGCCATTCAACGC | | TCAACATAAACAGAAAAAGAGATCGTCATCCTTCGAAA[C/T] | | | | | |
| CCCTCACAGCCCCGCTGCCTCGCGAGT_NODE_1_length_557_ | | ATCAATCTAAGCTCTCCTGCTTTCGATCCATTTTTAAGGAGAA | | | | | |
| cov_104.605026 | | TTCATAAGAGTAGAGAAAATTATTATGTGAAAATAAT | | | | | |
| RAD_kmer_0003079_1_TGCAGGAGAGGGTATTCGGACGTG | 428 | GAGTAGAGAAAATTATTATGTGAAAATAATAACTAAAGTGGT | A | A | T | A | GYSNP0905 |
| GCACCACCGTGCCCCTCCCTGCGGCCCCGCCATTCAACGC | | TTATAAGAGAAACGTGATTTCCTTTAATTATTTTCTTT[T/A]TT | | | | | |
| CCCTCACAGCCCCGCTGCCTCGCGAGT_NODE_1_length_557_ | | TCCTTTGACTGCTTTTAAACGGTGATAAAATAAAAACTGTCTG | | | | | |
| cov_104.605026 | | AAAATCAGTTGGGTGAAGGACTCGCGAGGCAGCGG | | | | | |
| RAD_kmer_0003090_1_TGCAGGCTACTCAAATAACACATCC | 520 | CTCCAAAGAGGCACAAAAGTCTGATTAAAAAAAGAAAAAAA | Y | Y | Y | Y | GYSNP0906 |
| AGCTGGTTGTGCCAATGTTTTGGGTGCCAAAGGCAATGGA | | ATCAGGAAAGGAAAACATGAATGATTGTATGGTATGTTA[C/T] | | | | | |
| TTGGGCTGGTTCAGTACCCTCTTAAT_NODE_1_length_606_ | | TTGTAGTTAGACCATTACATGATTAAGAGGGTACTGAACCAG | | | | | |
| cov_158.711227 | | CCCAATCCATTGCCTTTGGCACCCAAAACATTGGCACA | | | | | |
| RAD_kmer_0003092_1_TGCAGGTGAATAACTCAACATAAAA | 205 | TTCAGAAATTGTGTCAGACTAGCAATTTTATATTTTTTTTACC | A | G | A | A | GYSNP0907 |
| AACAGGTGCCACCTTTAATAGCAGGCGTTCAGTTGTCATA | | TGAGACGAACATGTTTATTGATTTGGAATTTGTCTCT[A/G]TT | | | | | |
| ATATGTTGTTGATCAGACTTTGTAGT_NODE_1_length_556_ | | GTGATCGTAGGTTGGTTGTAAAATAAATTCGTTCCTTCACTGC | | | | | |
| cov_71.924461 | | TATCATACGGTTGATGGAACAGAGGTAGTCGGTGA | | | | | |
| RAD_kmer_0003092_1_TGCAGGTGAATAACTCAACATAAAA | 272 | GGAATTTGTCTCTATTGTGATCGTAGGTTGGTTGTAAAATAAA | G | R | G | R | GYSNP0908 |
| AACAGGTGCCACCTTTAATAGCAGGCGTTCAGTTGTCATA | | TTCGTTCCTTCACTGCTATCATACGGTTGATGGAACA[G/A]AG | | | | | |
| ATATGTTGTTGATCAGACTTTGTAGT_NODE_1_length_556_ | | GTAGTCGGTGATTGCTCCTGCAATATTGGTAAGTAATATCTA | | | | | |
| cov_71.924461 | | ATTTCATTTTTGTTGATTTAGACTCTGCATTGATGA | | | | | |
| RAD_kmer_0003094_1_TGCAGGCCTAGGTATTTACATCCGA | 165 | CTTTTCTACATTTAAGTTTGTAAGTCATGACCCATAGAGAGTC | T | G | T | T | GYSNP0909 |
| ATCGCTGATCCATTATGGTCTCTACTGAATATTAGAATTTA | | CTTGTTAGTCACAATATTTCAGATATGATCATCAAAG[T/G]TG | | | | | |
| CAACAGCAAATAACCATATTAAGTT_NODE_1_length_603_cov_ | | TATTCCATGTACTCAGATCCTATGTCTAACCCACTTAATTTAC | | | | | |
| 99.592041 | | CATTTAGGAAAATACTCTTATTACCATTGTAACAG | | | | | |
| RAD_kmer_0003097_1_TGCAGGAATGAAGGTTCTGTATTTT | 180 | TACAAAAAAGATTCTTATATACAATCCCTTTTTTTTGGTTTCT | T | T | C | T | GYSNP0910 |
| TTTTCTCACATTTAGTTGTGAACATGCTTGATGATTCGAGC | | GAAAACTGATTCTTTCAGATATGTATAGAGTTACTAA[C/T]TG | | | | | |
| TATATATAAGAGTTTAGTTTGTTGC_NODE_1_length_269_cov_ | | ATAAAACACAAGAGAATCTAACCAAAGAAAACTGAATACCT | | | | | |
| 15.892193 | | TCTTCCCCTTGGAGGCTTATTGACCTCGGTAACAAGT | | | | | |
| RAD_kmer_0003098_1_TGCAGGAACCAAAAAGGTAAATTA | 113 | GACGACCAACAATGCTCCGCCCATGTATTAGACCATTTAATT | R | G | R | A | GYSNP0911 |
| TAATATTTTGGTTTTGTGAATTATTAATCTAGAAATGACAT | | TACACAAGTGTATAAATAAAAGAAATACAAAGAAAAAT[A/G] | | | | | |
| TTTGAAATACGTGAACTAAGTGGATG_NODE_1_length_226_ | | ATATAACTCATAAATCTGTAAACAATGATGGTCAATATGTTT | | | | | |
| cov_107.707962 | | CCATTTTTGTAGCATACCAACAATTATAGTGTTTTTTG | | | | | |
| RAD_kmer_0003099_1_TGCAGGAGAGTTGACAGCTCCTCCA | 464 | TTGGGTCAAATTTGTTTCCAGGAAACAGAGGAAATGATAGCA | A | A | W | T | GYSNP0912 |
| AGTCTTCGACTGATGTGTGCGGATGAACCAACCCACCTCT | | GATGTTCTTGGTGTAGAAGTATTTAGACAGACAATTGCT[A/T]G | | | | | |
| ATTTGAAAATGCACAATAACTCCCCA_NODE_1_length_542_ | | GGAATGTTCTTGTGGGGAGTTATTGTGCATTTTCAAATAGAG | | | | | |
| cov_110.959412 | | GTGGGTTGGTTCATCCGCACACATCAGTCGAAGACTT | | | | | |
| RAD_kmer_0003100_1_TGCAGGCTGACATAACAATGAAGTT | 335 | ACGCAACCGTTGATGGGGAAGAGAGGTACCAAGATGATGGA | R | A | A | R | GYSNP0913 |
| GTACTTTATTTTAATATGAAACAACATTTAACATCAACCG | | TACTTCCTATGAGTGGTAGCTTCCACGGCCCGGGGGCTA[A/G] | | | | | |
| AATGCATCTATATATACCTTCCTCTA_NODE_1_length_596_ | | ATTTGGGATTGACTTTTTTGTTAGTTTTAGTAACATGGATATA | | | | | |
| cov_79.748322 | | AAGATGAGAAAGGCAACCAAAATTTGTAACGGGTGAA | | | | | |
| RAD_kmer_0003104_1_TGCAGGTTTTCTAAGAGCCCAAAAG | 181 | GGCTGGCCTTGGAATCTCGCGAGCTCTCTTCTTCATTGTCAAC | T | A | A | A | GYSNP0914 |
| AATGGCAACCCAGATAACTCTAGACCCAAAGCTAACTCG | | GCATGGTTCGTCGCTTTTATAGGATCGTCGCCCGATA[A/T]CT | | | | | |
| GCCACCTCGCTTTTGCTCACCATAACC_NODE_3_length_421_ | | TGATAAATAATTCGGATGGTCGAACAACGGCTGAGGATTTCT | | | | | |
| cov_30.520189 | | TGACACCGCCGAAGTGGATTCCTTTTCCGAGTAAAC | | | | | |
| RAD_kmer_0003104_1_TGCAGGTTTTCTAAGAGCCCAAAAG | 273 | ATTCGGATGGTCGAACAACGGCTGAGGATTTCTTGACACCGC | C | A | M | M | GYSNP0915 |
| AATGGCAACCCAGATAACTCTAGACCCAAAGCTAACTCG | | CGAAGTGGATTCCTTTTCCGAGTAAACTATGCTACCGA[C/A]A | | | | | |
| GCCACCTCGCTTTTGCTCACCATAACC_NODE_3_length_421_ | | GTATGAGGCCAATTGGATGATGGACGGTCTTTCTGTTAATGC | | | | | |
| cov_30.520189 | | GTCGGGTGTTTCGGATGCGTATCGTTCGGGAATGGTT | | | | | |
| RAD_kmer_0003105_1_TGCAGGTTCTTTACCCCTGCACACTT | 561 | GCTGTTCCGACTTCAAGGGTGATAGGCACGAGGTTTTTGATG | G | A | R | A | GYSNP0916 |
| CAGCCAACAACCACCTCTCTCCTCGCTTCTTTTGTTCCCTT | | TTGCTAACAGTCATGGCAGGGTGAAACTTGGAGTCCAT[A/G] | | | | | |
| TTCTTTCTCACAGCAGCTATGGAC_NODE_1_length_619_cov_ | | GCTGCTGTGAGAAAGAAAAGGGAACAAAAGAAGCGAGGAG | | | | | |
| 68.969307 | | AGAGGTGGTTGTTGGCTGAAGTGTGCAGGGGTAAAGAACC | | | | | |
| RAD_kmer_0003112_1_TGCAGGTGATCAAATACATTTAAAT | 199 | TGTCGCGAGGTTAACACAGATGCAAGCCTTATAGGAGAAGGT | K | — | T | T | GYSNP0917 |
| TTCCTAGTCTTTATGTCAACCTTTTGAATCCTTACGTTCAT | | TTATCAACCACCTATAGAGAAAAAGAAACAACGTAAGG[T/G] | | | | | |
| TTCCTTCACAAGTTTTTATTTTCTT_NODE_1_length_656_cov_ | | AATAATATCATATTCCATGTACTACATGTGACGAGTGAGTCA | | | | | |
| 126.795731 | | TCTTAGAGGCAATAGAATTGGTAGTTGGTATGCAATAC | | | | | |
| RAD_kmer_0003112_1_TGCAGGTGATCAAATACATTTAAAT | 375 | AAATTCCATACTGAACTGAAGGTGCATTTCCCACACCGCCAA | T | — | T | Y | GYSNP0918 |
| TTCCTAGTCTTTATGTCAACCTTTTGAATCCTTACGTTCAT | | CTGACATTTGAGGCATTGCTGGTAGAGTCCCAAAAGGA[T/C]T | | | | | |
| TTCCTTCACAAGTTTTTATTTTCTT_NODE_1_length_656_cov_ | | TGTAATAGACTGCGGTTGTGGGCTCTGATTGGCAGGCCTGCC | | | | | |
| 126.795731 | | ACATTAAAAATAGGAAAGCATTGAGAAGTTCCCAACA | | | | | |
| RAD_kmer_0003116_1_GCAGGCACATTTTTTTAATTTATAA | 224 | TAACCTCGGATATTGTTTCAAAATACAACACCTATATGTATC | A | A | G | G | GYSNP0919 |
| ATTAAGGGTATGTTAATTAAAGACAGGATATTTAAAAGTA | | TTATCCCACCGATACCTTTCTGTTAGCTTCTTACC[G/A]TTA | | | | | |
| ACTTTTAAATATTACAAGGGACTGT_NODE_1_length_555_cov_ | | TGTAGAATATGTTATTGATGTTCACACATTCTTGTCTTTTGGC | | | | | |
| 91.875679 | | CAGTTAATAATATATGCATTTTGTTATGTACTTA | | | | | |
| RAD_kmer_0003120_1_TGCAGGTACATCTTTTTTACAATGTT | 331 | GTAGTCATAACAATGTTATTAAACTGAGGTTTAAAACCCAAT | — | C | S | C | GYSNP0920 |
| TTAGTAGCACTGACTTGAGAATGTTTTCTTCTTTATGTCAT | | CATATTTGTACTAAATACCATATTACACAAAATCAAGA[G/C]A | | | | | |
| AATCTTTTCTGGGTCCCACAGCTG_NODE_1_length_534_cov_ | | TAAAAAGAGTAAAATTTTAGATAAATATGTGAAGTGCAATAC | | | | | |
| 143.539322 | | CTGGTTTCTTGAGTGCCTCTAAATGCTATAACAATAG | | | | | |
| RAD_kmer_0003130_1_TGCAGGGGGTGGCAGCAACAGCAA | 351 | AGCAATTGAAAACATATCAAACAAGTTACACTTATCAATCCA | A | W | A | W | GYSNP0921 |
| CAGCAACAACACCACCAGTAGCACAACCAGCTCATCCGA | | ACACTACCTTTTATGTCGTGTGTAGAATTTTCACCTAA[A/T]A | | | | | |
| TGAGGAGACCCTCGAAGAAGCTAGAAGA_NODE_1_length_ | | AAATATGCATTAATTATATTCCTTTCATCCTTTTTAGAACATG | | | | | |
| 606_cov_165.981842 | | ACGTGTTTCTTCTATGCCTGAAGCTGTAAACTCCAA | | | | | |
| RAD_kmer_0003135_1_TGCAGGAAGTCAAAATCATGTGTGA | 133 | TTTTATATATATTATAGTGCATTAATATCTATGCGACATCTTA | C | Y | T | C | GYSNP0922 |
| AAGTGTGAAACAGGCATTTAAGCTTAAAAAATTGCATTTT | | ACAAACATTTTATCACTTTATATTAGATTTGTCTATC[T/C]CGG | | | | | |
| CTGATACCGAGGAATTTGAGATTGTT_NODE_1_length_557_ | | AAAGCTTCCGGGATATAACCTAGTAGTTTGATATGTAAAACA | | | | | |
| cov_122.166969 | | TATGAGAAAGACGCAACTTCTGGTTTAAAATAGAT | | | | | |
| RAD_kmer_0003135_1_TGCAGGAAGTCAAAATCATGTGTGA | 423 | TGTGTAACCTGCCTGCTAAATTGTTCTCGTCATTAAAATTTGT | M | A | A | C | GYSNP0923 |
| AAGTGTGAAACAGGCATTTAAGCTTAAAAAATTGCATTT | | TATCTTAATTCAGGTCAATATACCGAGTTTGCTTCTA[A/C]CA | | | | | |
| CTGATACCGAGGAATTTGAGATTGTT_NODE_1_length_557_ | | CGCATCAAAATGGACGCCCTACAGACAATGGGAACAGTAAG | | | | | |
| cov_122.166969 | | ATCATTGCTTATATCATACTTTTTTAAACAATCTCAA | | | | | |
| RAD_kmer_0003136_1_TGCAGGACCGTCAATAGACGGAAT | 318 | TGGCTTCAGTTGAATCCAGTTTTGCAAGCTTCCAAGCACTGAT | G | G | A | R | GYSNP0924 |
| GGATCAACAAAGCTTGCAATCTTCTCCAACCAGCTCAGCT | | TCGAACTGGACGGTTAGGTGGTCGTTTACCCTTGTCA[A/G]GT | | | | | |
| GTCACTGCCATGAGTGGCAGGAGCTCC_NODE_2_length_446_ | | GGCTGGACCGCATCTGGATCCACCGTTGATGGAAGCCGTCCA | | | | | |
| cov_32.984306 | | GGCTCTAGATGGGCTATGACTTCATCCTGATGTTAA | | | | | |
| RAD_kmer_3003139_1_TGCAGGCTTTCTATTAGTTAAATGA | 87 | CTATGGTCATATCAAAACAATTTTATTTAACCATGAGACAGG | A | C | C | A | GYSNP0925 |
| AGTTTATCAAGTAGTATTATGTGGTGTGTTTTTGCTTTTAC | | CAAATCGTTGGTTGCGTATCTACATTTACTCACCTAAA[C/A]C | | | | | |
| TATGTCAATGGTGATTACCATGATA_NODE_1_length_575_cov_ | | CTGAGTATATCATACTGTAAGCGGTTAATAATAATATATATTT | | | | | |
| 83.224350 | | GATACAATGTCACGTAAATTCAATATCAGTGGGAAA | | | | | |
| RAD_kmer_0003139_1_TGCAGGCTTTCTATTAGTTAAATGA | 322 | ATTAGCCATGAAAAATCATCATCAAATTAAGCATTCATGCTT | — | A | T | T | GYSNP0926 |
| AGTTTATCAAGTAGTATTATGTGGTGTGTTTTTGCTTTTAC | | GTCTATCCCATGAAAAATCTGATGGTATCTTTTTTGTA[T/A]A | | | | | |
| TATGTCAATGGTGATTACCATGATA_NODE_1_length_575_cov_ | | ATCGCGGTATCTAGTTGACTAAACCCACAAGATCTGAAGCTG | | | | | |
| 83.224350 | | TAGATTTATGGTAACAGCATCTTAAGGTTTGGTACTC | | | | | |
| RAD_kmer_0003139_1_TGCAGGCTTTCTATTAGTTAAATGA | 385 | ATGGTATCTTTTTTGTATAATCGCGGTATCTAGTTGACTAAAC | — | C | T | C | GYSNP0927 |
| AGTTTATCAAGTAGTATTATGTGGTGTGTTTTTGCTTTTAC | | CCACAAGATCTGAAGCTGTAGATTTATGGTAACAGCA[T/C]CT | | | | | |
| TATGTCAATGGTGATTACCATGATA_NODE_1_length_575_cov_ | | TAAGGTTTGGTACTCACGGCCTGGTGCGTTTACACGAAACGA | | | | | |
| 83.224350 | | TTGAAAAATACAAAAAATACTTAACCAACAACATAA | | | | | |
| RAD_kmer_0003142_1_TGCAGGCATGTGGTCTGAAGAATAT | 193 | AAATAAATAACTTGTTTAAATTTGTATCCAGGTGATGCATTTG | G | A | G | A | GYSNP0928 |
| TAAGATGTTACTGAGGATGTTGGTTGTGGTTTATGAACTC | | TTGGGGGATTTTTGTCTCAGTTGGTACTTGAAAAGCC[G/A]AT | | | | | |
| GTGGTGTAGCGGCACATATCAGAAAC_NODE_1_length_653_ | | TGCGGAATGTGTGAGAGCCGGGTGCTATGCATCCAATGTTAT | | | | | |
| cov_182.837677 | | TATCCAAAGGTCAGGGTGCACCTATCCAGAGAAGCC | | | | | |
| RAD_kmer_0003142_1_TGCAGGCATGTGGTCTGAAGAATAT | 578 | ACGTCTGAGGTATATACAACAGTTAATAAATTTTGTTGCAAA | T | C | T | C | GYSNP0929 |
| TAAGATGTTACTGAGGATGTTGGTTGTGGTTTATGAACTC | | CTTGCAAAGGCTGAGTGGTTGATTCTGGGTTATATACA[T/C]T | | | | | |
| GTGGTGTAGCGGCACATATCAGAAAC_NODE_1_length_653_ | | GTATGATGTGTTTCTGATATGTGCCGCTACACCACGAGTTCAT | | | | | |
| cov_182.837677 | | AAACCACAACCAACATCCTCAGTAACATCTTAATAT | | | | | |
| RAD_kmer_0003144_1_TGCAGGCTGCAACCTAAATTCACAC | 130 | ACATATGCCTCACCTTGCTTCTCCCGGGGGATCGAACTTGCGC | C | T | T | T | GYSNP0930 |
| TTGACACTTTAATCTTTCTTTCATTTATGTATGTCAACATT | | CTCCAAGGAGGAACACAAGCCAACCAACCGGTTGATC[T/C]G | | | | | |
| GGTTTTTGTCAAAATGGAACTAAGC_NODE_2_length_474_cov_ | | GGGATATTGACATAACCATTTGTGTAACTAACTACCAAAAAT | | | | | |
| 61.016876 | | AAAAACTATTACTTTTTGTAACTCTACGTGTATAGTT | | | | | |
| RAD_kmer_0003146_1_TGCAGGGGCTGGTGGAACGCCATG | 145 | TCAATTGACATTGATTGCTTGTATGATGGAACAGATTTTTCAA | T | G | T | — | GYSNP0931 |
| AAGAGTAAATTCATCAAGTAAGATGTTGTCTTTCACATTA | | TAAAGGTCACCCGGGCAAAATTTGAGGGGCTAAATAC[T/G]T | | | | | |
| CTACACTCACCCTGATGCACATCAACG_NODE_1_length_411_ | | GCTTTTTTAAGAAGTGCATTGAGCATGTGGAAAGTTGTTTAG | | | | | |
| cov_44.459854 | | CAGATGGGAATATACACAAAAACAATGTTGATGATAT | | | | | |
| RAD_kmer_0003152_1_TGCAGGTCCAGCAGCAGCATCTAAC | 466 | AGTTGGGTTCTTGCACAAGTGGGAAAAGCTCAAGTATGGCAT | — | T | C | T | GYSNP0932 |
| AAGTATAGAGGGAAAATATTACTATTTTATGAAATGAAA | | GCACTAAGGTTCCTTATATACTGCTGGAGCAATTCGTA[C/T]G | | | | | |
| AAATAATCAAAGCTCAATTTTGGAAGT_NODE_1_length_598_ | | AATGGGATCTATCAGAGGAACCCATTCGTGCGAATGACTCAT | | | | | |
| cov_100.981606 | | GGCATATCAAGAAAAACAAATTTAACTTCCAAAATTG | | | | | |
| RAD_kmer_0003159_1_TGCAGGACTGAATCAGAAATCGTAA | 350 | GAGTTTGCCCATGTTTAACACTTCTTACGGGTATGCTTTCATC | Y | C | Y | C | GYSNP0933 |
| ATGAAATCTTGACGAGAATCTTGCAGGACACGCAAGATG | | CTTTAAAACATCATCGAGGATTTTTTGTTGTAACTTG[T/C]ATA | | | | | |
| CATCACCGGTAGATCTTAGTCATCTTA_NODE_1_length_634_ | | AATCACTATCATGTTCCTTGGTGATGTCCTTTATATATCCAAT | | | | | |
| cov_127.227127 | | AAAGCTACTTCCTTGAAATCTCTTTCGAATGCCC | | | | | |
| RAD_kmer_0003162_1_TGCAGGTTATCTCCTGAAGTGATAC | 456 | AACAATAATCAATGTCATTACTACTGAATTTGACAAATACAA | K | — | G | G | GYSNP0934 |
| CGGTTCTGATGTTTAATAAAACTCGTTTTAACAAAAGTCG | | TTTAAAATTCTTTTAAAGAAATGGGTTAAAGATCACCG[G/T]C | | | | | |
| TTTCGAGAGTTGAGATCATAAGCTCA_NODE_1_length_561_ | | TGAATGGGGCTGCTATTACTAATGCCCCAGACCGAATGTTGA | | | | | |
| cov_169.946518 | | GCTTATGATCTCAACTCTCGAAACGACTTTTGTTAAA | | | | | |
| RAD_kmer_0003173_1_TGCAGGTAGACCATTTTGCCCTCAC | 152 | CAATTTCTTTTAGAAAAAAACAGTAAAATGTAGAGAAAGATG | G | R | R | G | GYSNP0935 |
| AATTGTGCCAAAACCGCGTAACATTCTAAAATGACCGCTT | | AGCTAGTTCTTACGGTGGTTGTTCGAGGTCTAAAGTGA[G/A]C | | | | | |
| TACCCTCACAAGTTTATACATTTTAT_NODE_1_length_453_cov_ | | ACAATCTCTCAACCTAGCCAAAGGTAAACCTTTCTTTGATAA | | | | | |
| 163.125824 | | AGGATAACTCTCTTGATCAACTATCCCAATATGCAAC | | | | | |
| RAD_kmer_0003177_1_TGCAGGGTTGATCACATCAGTAATA | 459 | GTTGTCTTGCAAGACTGTGACATCCAGGCCCGTCGCCCCAAT | Y | C | Y | Y | GYSNP0936 |
| GACGATTGCATCACAATTGTTCTCGAGTACAACTTCCACG | | CCCAAGCAAAAGAATATGGTCACAGCTCAAGGCCGAAC[C/T] | | | | | |
| GCCTTCCAAGATACGTTGGAAAACTG_NODE_1_length_601_ | | GACCCTAACCAAAACACGGGCATTGTTATTCAGAAATGTAGA | | | | | |
| cov_123.156403 | | ATTGGTGCAACATCTGACCTCCAACCTGTTCAAGCCAG | | | | | |
| RAD_kmer_0003177_1_TGCAGGGTTGATCACATCAGTAATA | 537 | ACCGACCCTAACCAAAACACGGGCATTGTTATTCAGAAATGT | S | S | S | S | GYSNP0937 |
| GACGATTGCATCACAATTGTTCTCGAGTACAACTTCCACG | | AGAATTGGTGCAACATCTGACCTCCAACCTGTTCAAGC[C/G]A | | | | | |
| GCCTTCCAAGATACGTTGGAAAACTG_NODE_1_length_601_ | | GTTTTCCAACGTATCTTGGAAGGCCGTGGAAGTTGTACTCGA | | | | | |
| cov_123.156403 | | GAACAATTGTGATGCAATCGTCTATTACTGATGTGAT | | | | | |
| RAD_kmer_0003185_1_TGCAGGCTGCAGCATGTGTCATTGT | 541 | AACAAAACCAATAAATAACGATAAAATGCCAATATTCATTAC | G | C | C | C | GYSNP0938 |
| TTAACTATTATATTACTTTAAAGTTTTTACTTTGCTTATAG | | ATATTAATTAATAGCATTGACATGTTCATAAAAATTAA[C/G]C | | | | | |
| CTTTTGTTTACTTAAGGTTATTGGT_NODE_1_length_604_cov_ | | AATAACCTTAAGTAAACAAAAGCTATAAGCAAAGTAAAAAC | | | | | |
| 82.645699 | | TTTAAAGTAATATAATAGTTAAACAATGACACATGCTG | | | | | |
| RAD_kmer_0003187_1_TGCAGGACAAGAAAAATATTTACCA | 137 | CCAGTAAGGCAGTAATAAACCTGATTATTTTTCACTGATGCA | — | R | A | R | GYSNP0939 |
| ATAGAAACTAAAAACATACATTATGTAATGACTATAATCA | | AACTAAAATTAGCCTCATAACATTAACTTTACCTTGTG[A/G]T | | | | | |
| AATATTCAAATAACAACCAATTATAA_NODE_1_length_596_ | | AAACAGAAGAAAATGCCGACCAAGCAAACGGCAAAATAGAA | | | | | |
| cov_103.889259 | | AGACAGCAGTTGTCTTCTGTTTCAACAGCTTTGCTTTT | | | | | |
| RAD_kmer_0003193_1_TGCAGGTAGAAAAACTCATCAAATA | 144 | TGTCACGAGATGTCTTTGATTTGTTTCTATGTTTCCTTTCTAAA | A | R | A | G | GYSNP0940 |
| TAATCCTCTTCCGAGCTAAATCAGCCTCACTAAAATTTGTT | | CCAGATTAAGTTTTTTAGGGAACACTGTGGTATCCA[A/G]TGT | | | | | |
| GGCTGGTGAATCCCAATATATGCCT_NODE_1_length_614_cov_ | | TCCAATATCAAAGTAGCTTCTGCACAATTGATGTTTTCCTCAT | | | | | |
| 123.912048 | | ACCTTTGTAGCCTTATTCATTTGATCATTTATGT | | | | | |
| RAD_kmer_0003201_1_TGCAGGCTGCACCTCATGTATAAGT | 319 | ATCTTGGAAAGAAACCAGATACATACGTACCATAAGTTTTTG | C | Y | Y | T | GYSNP0941 |
| ATAATATAACTTGGTCAACCGTTTTACCGATTGAAATCAA | | CAGATGTATAGTTAAAAGGCATGACACGTGGAACAATA[T/C] | | | | | |
| GATTTGTATTAGATATGGAAGTGATG_NODE_1_length_622_ | | TGTCGGTTAGTTTGATTGGTTTGAGTTTGGCCTGATGTGTCAT | | | | | |
| cov_102.881027 | | CTGCACCATCTCCTTCAAGTCTCCAAACAGAAACCGG | | | | | |
| RAD_kmer_0003205_1_TGCAGGTGGTGTCAACATCGTCGTA | 421 | CAGACATGGATACATTGAGCCACGCAACTGTAACGACACCTG | A | W | W | A | GYSNP0942 |
| CTTTTAGCTTGCATAGTTGTACAGCCAGTGACATGAGTTT | | CAGTCTCTCTTGGTCAAAGTCAAGTAAACTTAGGTACA[A/T]A | | | | | |
| GACCTGGAGAGACTGCAGCTGCAGGT_NODE_1_length_598_ | | CAAGCAGGCTCAAAATACAACCGTGTTGACGCCTACTGCAGG | | | | | |
| cov_92.068565 | | CTCTCTTGGTCAAACTCAAGTCAACGTATGTAAAACA | | | | | |
| RAD_kmer_0003205_1_TGCAGGTGGTGTCAACATCGTCGTA | 497 | TACAAACAAGCAGGCTCAAAATACAACCGTGTTGACGCCTAC | M | M | M | M | GYSNP0943 |
| CTTTTAGCTTGCATAGTTGTACAGCCAGTGACATGAGTTT | | TGCAGGCTCTCTTGGTCAAACTCAAGTCAACGTATGTA[A/C]A | | | | | |
| GACCTGGAGAGACTGCAGCTGCAGGT_NODE_1_length_598_ | | ACAAACCAAAGTCAGGGTACAACAATGTTCACACCACCTGCA | | | | | |
| cov_92.068565 | | GCTGCAGTCTCTCCAGGTCAAACTCATGTCACTGGCT | | | | | |
| RAD_kmer_0003215_1_TGCAGGCGGGTTGCCACCAATGTTA | 110 | TGTTTTTTCATAAGCAAGCGCAATCGAACAAACACATAAATT | G | G | R | R | GYSNP0944 |
| AACCGCACCTCTAGTAAGGCAACATCTGCTCAAAAGGGA | | TGAATTTACATGTCGAAAAAGATACTAAGCCGAGCATC[A/G] | | | | | |
| CCGCCAGCTGGAGGGGCGCCAAAAATG_NODE_1_length_576_ | | AGGTCTTTCTTTGTCATCTCCTCCCAAAAATCTTTAACTTGTTC | | | | | |
| cov_154.821182 | | TTTAGTGAGCCCGTCTTCGACATCTATATTCCTACG | | | | | |
| RAD_kmer_0003228_1_TGCAGGATTTAATCCGCATGGCGCG | 227 | GTGTAGTGGTTTGAGGTGCATTTCCCTAATGGGAGGTCACAT | T | — | Y | T | GYSNP0945 |
| GGCCAGTGGGCTTAATGACCCCGCGTGCAATGGTGTTTCC | | GTTCGAGACTCGGTTTGAAGGAAAGTGTCTTTTTTTTT[C/T]CA | | | | | |
| CATTCAAACAACTAATATTCCCACCG_NODE_1_length_577_ | | AGGTATATTAAATATAACAAATGGTATTTCATGTTAAACATA | | | | | |
| cov_128.161179 | | ATTGGTTTCATAATAAAACACAAATTGGGTTCGTGG | | | | | |
| RAD_kmer_0003229_1_TGCAGGAAGTTTAAGCCACATCTCA | 125 | CAAGTTATGAATATGTACCTTCTAAGGTAAGATCATGAAGAT | T | C | Y | T | GYSNP0946 |
| TAGGTTTGGGTCCAAATTGAATTAACAAATGGGCATATTA | | GCTTACTCTCAAATCACTTTTTTTGGAATTTAAACATC[C/T]AG | | | | | |
| ATAAAACATGGTCTGACGTCTCCTCC_NODE_1_length_632_ | | CACAATTCACCCATAGGATATCTGATAATCCATCGTTGGATTC | | | | | |
| cov_138.335449 | | CCCCATTTGAATAATGATGTATTAATCCAGATTGA | | | | | |
| RAD_kmer_0003232_1_TGCAGGTTCACAACAAGGATGAATG | 320 | GTATAACTTCAGGTAGCCGGAGGGGATCCCCTTCTAAAGCTT | Y | T | Y | Y | GYSNP0947 |
| GAAGTTTTTTCCCAGCTTCCGACTGTGACACAAAAAATGG | | ATAGCAGTTGACTGGTTTAAACTTGAAACTTCCAGAGG[T/C]A | | | | | |
| ACTTAGCATAACCAACAGAATTACGG_NODE_1_length_582_ | | AGTGGCTTTGCATCCCAAATGCCTTGTTCAGGTACTATATTG | | | | | |
| cov_112.609962 | | ATCATTTTTTATGGAATACTTTGTGTAACACAGATA | | | | | |
| RAD_kmer_0003235_1_TGCAGGTTTGTATTTTTCATTTAATC | 432 | AAGCCCATAATTAGTTATAAAAGCTGGTTCCATTAGAATTTA | M | C | M | C | GYSNP0948 |
| TTATTTATTGAGATGTCAAGCTTGGCATCCACCCCCTCTGT | | TAAAATAAATGAAATGAATTAACCTACCTGCAGCCTAG[C/A] | | | | | |
| GCTCGATCTTTATCTACTTATTAC_NODE_1_length_626_cov_ | | AAAAGCTTCCTTGCGCTTCTGGTCCTCCCCAAGTGACCATTGA | | | | | |
| 123.880188 | | TATTTTAAGTATGCATATATTACTTGTGGTGGCCCAT | | | | | |
| RAD_kmer_0003238_1_TGCAGGCACGCAGTTACAAGGTGTT | 303 | GTTTATTCTCGAGTTGGGGGTCTCTCTGGAAGCAGCCTCTCTA | C | M | C | M | GYSNP0949 |
| ACACCACATTGATGGCACTGAACCGCCTGCCAAAACAGA | | TTCCTTGGTATAGAGGTAAGGTTTGCCTACATCCACC[C/A]TC | | | | | |
| TCCTGACTACGCTAACTGGGCCAAAAT_NODE_1_length_577_ | | CCCAGACCCTATTAATAGCTTTGCTATTGGTGGGATTTTACTG | | | | | |
| cov_142.596191 | | GGTACGATTGTTTATTAAAGTGGGTTTGGATAGGG | | | | | |
| RAD_kmer_0003243_1_TGCAGGTGTACCTAATATGTTACCG | 587 | CGGCTTCTAAAAGTATAAGAGGTAACATCATTTATCATCATA | C | — | T | C | GYSNP0950 |
| ACTCATTCCTAAGAACGCTTACTCATTTCGTTAAATCTTTT | | AATTTCCAACTTAATTCCTTTTTCAGAAGAGAGTGTCA[T/C]G | | | | | |
| GTGTATTTTATCACTAATATAATGT_NODE_1_length_688_cov_ | | ATCTTAAAAATATTTGTTTCCTGATCAAACCGAAAACATTATA | | | | | |
| 77.991280 | | TTAGTGATAAAATACACAAAAGATTTAACGAAATGA | | | | | |
| RAD_kmer_0003252_1_TGCAGGGTTTTnCCAACATATATA | 139 | CCAATAATACACGGCTGCTTACTTAGAAGCTAGGCTCTATGT | C | A | C | C | GYSNP0951 |
| GTTGTTTTGTCGCAAATGCGTAGGAAATCCATGTTTGCAA | | CTATCGTGATTAGTATTTTCTTTTTATATTAAAACAAT[C/A]TA | | | | | |
| TGTATTTCTCACGAAAAAAGTGAGTC_NODE_1_length_534_ | | AAAAATAATATAGTTTCGCGTTTTTATTGCAAGCAAGTGATT | | | | | |
| cov_98.808990 | | GATATGTTATTAACAACTATGCTCTATCTCTCACTG | | | | | |
| RAD_kmer_0003258_1_TGCAGGATCGATTGAGTCTTTCATA | 400 | GAAACAAAAATTAGATTATCAGTAAAAAATTTAAAGAATCA | A | W | W | T | GYSNP0952 |
| GAGGTACAGCAGAGAATGGGGCGTTTGGTTAGATCATGC | | AGGTTGTTGTCAAACCGGTGCTTTAATTATGTCTGCGAG[A/T] | | | | | |
| AACTTTAAATCAACTCCACATGTTGAT_NODE_1_length_534_ | | ATCAAGCACACAGTCCTTGAAAAACTGTGAGAGCTGAGTCGT | | | | | |
| cov_157.155426 | | GTAGAGCTCGGGCTCATGCCTAAAATGATCAACATGTG | | | | | |
| RAD_kmer_0003261_1_TGCAGGCTTCACTATCTTCATAATTT | 138 | GAAGGGGTATAATGGCCAACAAATTATATAAATAGACATGCC | S | S | C | C | GYSNP0953 |
| CCACACCATTTAAAATCTCGTTCTTCCTCACAACTAGTACT | | AAAGCTTTGATTATGTTGTAATAGATTACACATGATTT[C/G]T | | | | | |
| CAAATCCGACAGGCCAACTCCCCG_NODE_1_length_632_cov_ | | ATTTACTTGAAAACTAAGTTTTGTTCCGTTTATCACAACTCAT | | | | | |
| 113.767403 | | GCTGTAGTACTTGTTGCTTCCTTGTATCTTGGGAAG | | | | | |
| RAD_kmer_0003261_1_TGCAGGCTTCACTATCTTCATAATTT | 290 | CTTGGGAAGCGAATTAAATGTAGGTGGTTCATGGTTCAAAGT | C | — | M | C | GYSNP0954 |
| CCACACCATTTAAAATCTCGTTCTTCCTCACAACTAGTACT | | TAAAACCTTTTAACGGCACCCTTTGGAGAGTTGGGTTC[A/C]C | | | | | |
| CAAATCCGACAGGCCAACTCCCCG_NODE_1_length_632_cov_ | | GATGAAGAGTTTTTAACAATAAAATCTGCAAAAATTGTTAGT | | | | | |
| 113.767403 | | ACTGAATCAGTTTTGTTTAATTTAACATGGGGGTTTT | | | | | |
| RAD_kmer_0003261_1_TGCAGGCTTCACTATCTTCATAATTT | 371 | CGATGAAGAGTTTTTAACAATAAAATCTGCAAAAATTGTTAG | C | — | C | T | GYSNP0955 |
| CCACACCATTTAAAATCTCGTTCTTCCTCACAACTAGTACT | | TACTGAATCAGTTTTGTTTAATTTAACATGGGGGTTTT[C/T]GG | | | | | |
| CAAATCCGACAGGCCAACTCCCCG_NODE_1_length_632_cov_ | | TTGATTCGGATCGCAGTGTTCGGTATTAGTTAGATTGCATTTT | | | | | |
| 113.767403 | | TGTGATATAGGAAGCTTGTCATTGAATCAGTTGTA | | | | | |
| RAD_kmer_0003263_1_TGCAGGAGAGTATATGAGATCCTTA | 249 | AGGCGACCGAATTCACTCCTGAAACTGCCTCCAGGTGATGTA | — | R | A | A | GYSNP0956 |
| TAGAAAAAGTAAAAGGCACTTAGTTCATGCAACACTCAT | | GGATTTGAACAATACACCACATCCCATTCTGCATACAA[A/G]T | | | | | |
| ATTTCGAATAATCAATTATTTGAAAGT_NODE_1_length_465_ | | ACATGAATTGGTGAGATTTCAGCAGAATCATTAACACTAAGA | | | | | |
| cov_121.587097 | | ACAAAAGCTGTTGCTACAAAATAAGGCCAGGGTCGGC | | | | | |
| RAD_kmer_0003267_1_TGCAGGTAAAGACATAACATTTGTA | 310 | GGACCTTGCCATACAGCTTGAAATTGGATGTATATAAGAATT | A | T | W | T | GYSNP0957 |
| ACAGAGAAACAAACAAACACGGAGGTTGATGACGGGAGC | | AGTACATACACATCACTTTGTTTTGTTTCTATGGATCC[T/A]AT | | | | | |
| TTGAGAACCCATATTTATTTCTACAGC_NODE_1_length_536_ | | ATGAACCTGTTGAGTTCTTAGGGTTTAAACGTGGTAATTATGT | | | | | |
| cov_82.916046 | | TTCAGTGTTAGTTTGAGAGATTTACAGCATCACAT | | | | | |
| RAD_kmer_0003269_1_TGCAGGAAATCCGTATTTCCCCTAA | 236 | CAAAATGGAATTTGAGCTCACGGAGAAACTTGGCTGTTAACA | T | C | T | — | GYSNP0958 |
| TTTAGGCATTCGCATATGGCCATAAACACTCTCGATTTCA | | AACCCATACTTTCAATTGAAAGATTGAGATTTTATCAA[T/C]A | | | | | |
| CTCTATTCCAACATTCAAAAGATACA_NODE_1_length_641_ | | TAAAAAAGCATACCTTTAGGAATTGTTCTTGGATTTTTTTATT | | | | | |
| cov_84.702026 | | CGAATGGGTAAACTTTATTTGCTAGTGTTTGTTATA | | | | | |
| RAD_kmer_0003280_1_TGCAGGTGGGACTAAAGGATCGGG | 315 | CGAGCTTTGAGCGAGCAGATCTCAAGTCGCTCATGAGCGGCT | T | T | A | T | GYSNP0959 |
| TTTGTTTTCGTCGCCGGGTAGCAGTCCGAATAAAAATATG | | TAACTCGTTTCCACCGGTGTTTATGCAACACGTAGAAT[A/T]A | | | | | |
| CTGCGGTTCAAGACGGATAGCTCGGGT_NODE_1_length_620_ | | TAGAGCACAAAAGCATCATTAAAAGCAAACCCTAGTTGGCA | | | | | |
| cov_75.119354 | | AGATTCACACATTCACACTTGAACTACTCATTGACCCA | | | | | |
| RAD_kmer_0003292_1_TGCAGGATCTCCAAGTAAACGAGTG | 494 | CCCATCTATTCCTGATGAACCAATGTTTTGTGGGTCTGAGTCC | A | T | A | A | GYSNP0960 |
| TCAAGAACCTTGGAGAGGGTGTACTACTGTCGTCTCCTAAGC | | AGGTTCAAGCGGCTCGAGCCACTCAACGTCTTCTGCA[A/T]AC | | | | | |
| ACGAGTCAATGGCTAGTTTTGGGAGT_NODE_1_length_589_ | | TTCTAGCACTATGGGTTCCTTCCGGTTCACTCCCAAAACTAGC | | | | | |
| cov_185.045837 | | CATTGACTCGTGCTTAGGAGACGACAGTAGTACCC | | | | | |
| RAD_kmer_0003293_1_TGCAGGCACTTATATATTCATCATTT | 467 | AGACAAACTTACCTTTGATGGGGTGTCACCATGTTATGGGAT | W | W | A | W | GYSNP0961 |
| GAATGCCACAAGTAATTAGTGTATTAAATAAGAATACAA | | AAAAGCGTTGTCTATATGAATAAATGTAAAGAGTGTTT[A/T]C | | | | | |
| AACTATATTTTTGAAATAAAGGGTAA_NODE_1_length_638_ | | ATCATCTGACATGTTTTAATCCAGAACTGGAATGAATGAAAC | | | | | |
| cov_121.394981 | | AGGATACATATTTGGATGGCCTTGAAGAAGCAAAGTA | | | | | |
| RAD_kmer_0003300_1_TGCAGGGTTCTTTGTGAAGCGAGAT | 355 | TGTGTTTTTGAAGGTGGTTTGATAGGGCTGCCATTATAACCCA | Y | C | Y | T | GYSNP0962 |
| GCAAAAGGGACAAATAATCGGTCGTATAATGCAAGGGTA | | AGATGATACAGCCCATCTCCTTACAAACTCGCCAAAA[T/C]TA | | | | | |
| ACAAGGATGGATAGGACATCAAAAACT_NODE_1_length_613_ | | TGGCGAATTGCTACTGTGCATAGAGTCGAAGAGCTAAAATCC | | | | | |
| cov_147.526917 | | ATGATTAGACTGTTACCATTTGTGTCAACAGGAATA | | | | | |
| RAD_kmer_0003300_1_TGCAGGGTTCTTTGTGAAGCGAGAT | 507 | ACAGGAATACTATTTGTGATGGCATATTCACACCAAAGTAGC | M | C | M | A | GYSNP0963 |
| GCAAAAGGGACAAATAATCGGTCGTATAATGCAAGGGTA | | TTCACCATCATGCAAGCTCGAACCATGGACCGTCACAT[A/C]A | | | | | |
| ACAAGGATGGATAGGACATCAAAAACT_NODE_1_length_613_ | | CGAAAACTAGCTCGTTTGAAATACCTCCGGCTAGCATGGCAG | | | | | |
| cov_147.526917 | | TTTTTGATGTCCTATCCATCCTTGTTACCCTTGCATT | | | | | |
| RAD_kmer_0003304_1_TGCAGGTTTTGCCCGGTGGAGATTT | 103 | TCGTTTTTGGCCATCTATGGTGCACAAAACTGTAAACAACTC | Y | C | Y | C | GYSNP0964 |
| CCTAAGAGGGACACACCATTTGATTGGGCTATTGGATATT | | AGATCTCTTATTTACTTGAACAAAAAACTAACAGAAAA[T/C]T | | | | | |
| GGGCTTGTAGCTGGATGATGGGCGAC_NODE_3_length_206_ | | CCACAATCAATAGCTAATCTATCTAACACAACCAAATAGATC | | | | | |
| cov_17.446602 | | ATGAACTGGATGATATATACAAAGAACCCAAAGACTA | | | | | |
| RAD_kmer_0003308_1_TGCAGGCGAGCTAGACGGTGGACG | 106 | AAAGGGAATGGGTAAGGCAAACAAACTAACAAACTCATCAC | C | — | S | C | GYSNP0965 |
| GATATCTTTCGCAATATGGGGGCGAAAAGTATGTTTTTCA | | TAAAACTATGATCCCCAATGCAACATATTTGTTATCGGG[G/C] | | | | | |
| ATTCCATTCTTTGCTTGGTTTATTTAC_NODE_1_length_455_ | | TCACTAATTTCTTCACTCAGTGGAGCAATGGGCCCACGTTGTA | | | | | |
| cov_45.804394 | | AAAAGAATATCAATACGACCCAATAAAATGCCACGTC | | | | | |
| RAD_kmer_0003309_1_TGCAGGTTATATAGATGGGTTCCATAC | 374 | TAATGAATCTTCCCACTTGTTAGTCATCAGATACATGTTCCAT | C | C | T | — | GYSNP0966 |
| ACTATGATTTAAAACTGTTTTCAACATGTTATGCCTGATTC | | AGAAGCATGTACACACCATCATCATACGGTTCTAACT[T/C]GA | | | | | |
| AAACAGTTCAAAACCCAAAAAATCT_NODE_2_length_512_ | | GCACCTTCTTTGCTGCTGCTTCAGATAATTGCAGGTCTCTTTT | | | | | |
| cov_61.591797 | | AATTAAACAAGCATTCAAAACCGACCTAAAAATAA | | | | | |
| RAD_kmer_0003312_1_TGCAGGAAATCCGTATTTGCTGATA | 400 | TCAGAACGCATGTGCCGAGATATTCTTGAAAATTTTTGTGAA | G | — | A | A | GYSNP0967 |
| AGGCTCGTATAAAAGCATAATGTCACTTGAGGTCGGAGTC | | GGTATATTTTTATTATTGTTATAATTCTTAATATAATT[A/G]TT | | | | | |
| CTCAAATATCGTCGTCCATACAGATC_NODE_1_length_493_ | | GTTAATATTATTTATTCGGTACATTTGATCTGTATGGACGACG | | | | | |
| cov_65.125763 | | ATATTTGAGGACTCCGACCTCAAGTGACATTATGC | | | | | |
| RAD_kmer_0003316_1_TGCAGGATCATACAAACCAAGACC | 246 | ATATCTCATTTTTTTGCTGAAAAACATGAACAAACCACACAC | A | A | A | T | GYSNP0968 |
| AATTTCTGATATTACATGATTTACTATTTAGCACAGAAAA | | GTGACTTTGGATATAACCTCAAAGGGAAAGCTATACGA[A/T] | | | | | |
| GGTTGACTCAAGAAAAAGATGATTTCT_NODE_1_length_603_ | | ATGTGCTGAAAGAATTGTAAGAACAACATACCTGCACTGAAG | | | | | |
| cov_130.041458 | | AGATCTATAGCAGAGCAATGGGCAGAAGAAAGGGTGGG | | | | | |
| RAD_kmer_0003318_1_TGCAGGTAATTAAAACAACAATGAT | 186 | TTATAAACATATTAGTTCTGTTTTTGGAATAAAAGATATAATT | C | A | C | A | GYSNP0969 |
| GCGATAAAAACTTAAATATTTCGTACTTATAGGCAAAAAA | | TTTTCGATTGACATCTGCTTTAGTATATTCCTCAGTT[C/A]TTA | | | | | |
| TGATCATTGAGTTCTTATTGCTTGCA_NODE_1_length_553_cov_ | | TTGTTTCTTTCTTCATATGATTGATCATTTAGTTTATAAAATTG | | | | | |
| 86.696205 | | CGTTTTTATCAATAGCCATGTCAAATAGATTGT | | | | | |
| RAD_kmer_0003324_1_TGCAGGCACATGTGATGTGTAAAAG | 115 | ACTTTTATCTAGGGAAAATTACTTTTATACGCTAATACACTAA | W | T | A | A | GYSNP0970 |
| GTGTCTGTGCTGCTGTACCGACTCCCCTACCCCAAGATCT | | TATTATTTTAAAACTTTTGATACTAATTAGATGATAT[A/T]AA | | | | | |
| AGAATTTTATAAACTTCACTCATCAC_NODE_1_length_560_ | | ATGTTATATAATAGACTTTTGTTTTTTTTTATTTTAAGAATAGT | | | | | |
| cov_117.930359 | | ATTATTTTTCTAATACATAATATATTTTTACTTC | | | | | |
| RAD_kmer_0003324_1_TGCAGGCACATGTGATGTGTAAAAG | 204 | TATAATAGACTTTTGTTTTTTTTTATTTTAAGAATAGTATTATT | G | — | S | G | GYSNP0971 |
| GTGTCTGTGCTGCTGTACGGACTCCCCTACCCCAAGATCT | | TTTCTAATACATAATATATTTTTACTTCTTTTCTTT[C/G]TTTGT | | | | | |
| AGAATTTTATAAACTTCACTCATCAC_NODE_1_length_560_ | | TTTTTTTAATCTCAGGCCCTCACTTTTTTTTTGGCCTTGCGCCT | | | | | |
| cov_117.930359 | | AGCGCCTTTGATAACTGTGCTTGATAGTCGG | | | | | |
| RAD_kmer_0003325_1_TGCAGGACACCTCATATTTATTAAA | 392 | AAGAAAACGCTTGAGATAAATACGCATTATAATCTAACAAAG | T | A | W | A | GYSNP0972 |
| TATATACTATATTAACTAACAAAACTAATTAATATCCTAT | | TTTGACATTGGATATTGAATGCGTTAAATGGTTAATTA[A/T]T | | | | | |
| TAAGAAGCAAGACAAACCATATCTGG_NODE_1_length_662_ | | GTAGGTACACCCTGGACAGAGGAGGAACATCGGTTGTTCCTA | | | | | |
| cov_115.453171 | | CTTGGTCTCCAAAAATTAGGCAAAGGAGACTGGCGTG | | | | | |
| RAD_kmer_0003331_1_TGCAGGCCCATGTGGTGGAAATTTG | 392 | TACTTTACCTTTTATTGGTGGCTCATAAGAAACTTTAATAAGT | K | T | G | K | GYSNP0973 |
| TATTTGAAGTCTGGCACTGACCGACGCAACTCCATGGACC | | ATGTTGTTATATCATCCTCAATTAAATAGAAAGTAAG[G/T]CC | | | | | |
| ATGATGCTGTATGATTCTTTGAACCT_NODE_1_length_602_ | | AACCTGCCTCGGCAAGTGTAAAGAGAAAGTAAGGCCAACCT | | | | | |
| cov_126.775749 | | GCCTCGGTAAACTTGGACACAGTATCAACATTGGTGC | | | | | |
| RAD_kmer_0003335_1_TGCAGGTTTCTCAATTCTAAAAGAC | 234 | AGTTTCTAGTCTTTTTGTCACATGTGTTCTAAAGGATGTTCTTT | A | C | C | C | GYSNP0974 |
| CCACATATTTCATGCCACATGCCATCATTAACCTCAACTTC | | ATTTAATTATTAGAATGTAGATTTTGGGGTTAATAT[C/A]TTA | | | | | |
| TCCAGCATTCAACCACTGGTCGTCC_NODE_1_length_592_cov_ | | GCAAACAGCGATCTACGTAGCGGAACAAACGGGTGGTTTCCA | | | | | |
| 80.697632 | | CTTGGTAATTGTGCGCTGAGTGTTGCAGCAAGCGG | | | | | |
| RAD_kmer_0003336_1_TGCAGGCATCTGGTGACAGTTTCCT | 495 | AATACTTCATTGTTAACGTTTGCTGATCTTGGGGATATGGTGT | T | C | C | C | GYSNP0975 |
| GCAATCCATTAGCCTGCAGATTCGTTTCTTTTCGCTCTTTC | | CAACTTTACCACGATCGACACACGATGGGATTTATCG[C/T]GC | | | | | |
| CTATCCCGGGACGTTCCTGTAAATA_NODE_1_length_654_cov_ | | AATTGACATGTATCTTAAGGTCAGTTTTTACTACTAAATTTCA | | | | | |
| 129.600922 | | ATTCTTTTGTTGGGTTTGGCTAATCAAACAACTGA | | | | | |
| RAD_kmer_0003336_1_TGCAGGCATCTGGTGACAGTTTCCT | 585 | ATGTATCTTAAGGTCAGTTTTTACTACTAAATTTCAATTCTTTT | C | T | C | T | GYSNP0976 |
| GCAATCCATTAGCCTGCAGATTCGTTTCTTTTCGCTCTTTC | | GTTGGGTTTGGCTAATCAAACAACTGACTTTATTTT[C/T]GAA | | | | | |
| CTATCCCGGGACGTTCCTGTAAATA_NODE_1_length_654_cov_ | | ATATTTACAGGAACGTCCCGGGATAGGAAAGAGCGAAAAGA | | | | | |
| 129.600922 | | AACGAATCTGCAGGCTAATGGATTGCAGGAAACTGT | | | | | |
| RAD_kmer_0003338_1_TGCAGGAGTAGGATTCTCATACTCT | 197 | ACTAGCTTCCATATGGATGTGTTCTTTTCTACCTTTAGTAATA | A | R | G | A | GYSNP0977 |
| AACAAATCCTCAGACTATCAAAAAATGGGTGACAAATCA | | GTCTTTAATTTAACAGAAGAAAACTGAACATATGACC[G/A]C | | | | | |
| ACAGCGGCAGACAATTATGTGTTCTTG_NODE_1_length_573_ | | ATAACATGGTTGGTGGTCCTGCCATGCAACATTTGTTGTTTGG | | | | | |
| cov_75.003487 | | CACCATATTGTATGTGGGGATGTAAGCCTTTCAACA | | | | | |
| RAD_kmer_0003340_1_TGCAGGTCTTTTAATTTGTACTATTA | 415 | GGATGTTCATCACCAAATGTCTAACCTTGGCTAAATGTGTGA | T | Y | Y | T | GYSNP0978 |
| AAAAAGAAAGTTTTTTTTTTAAACTTCTGTAAAATGGGGA | | CACAGGTTGCAGCCAGTGCTACTGCTAGTAATCCCACA[C/T]C | | | | | |
| TATGAAGTTAACGATCAAATTTGAT_NODE_1_length_685_cov_ | | CAAAGTCAGCAGCGATATAGTCCCTCCTGCTAAACCCAACCT | | | | | |
| 63.693432 | | GATTCTTCTTGCTGAAAAAAAGTAACAATTTAATAGG | | | | | |
| RAD_kmer_0003340_1_TGCAGGTCTTTTAATTTGTACTATTA | 480 | GCTAGTAATCCCACACCCAAAGTCAGCAGCGATATAGTCCCT | A | R | R | A | GYSNP0979 |
| AAAAAGAAAGTTTTTTTTTTAAACTTCTGTAAAATGGGGA | | CCTGCTAAACCCAACCTGATTCTTCTTGCTGAAAAAAA[G/A]T | | | | | |
| TATGAAGTTAACGATCAAATTTGAT_NODE_1_length_685_cov_ | | AACAATTTAATAGGCGTATAAACTATAAAGTAGGAGAAAGG | | | | | |
| 63.693432 | | AAGGCCCGGGGTTCAAGTGACGTATCTTTTTTCCAATA | | | | | |
| RAD_kmer_0003344_1_TGCAGGCAGTTGGAGTCACAGGTTG | 119 | CAAGGTTCTTTGGTTGAGTTCATGGTCAAAGAAACCGAAAAG | T | Y | Y | C | GYSNP0980 |
| CTGCAGAACCACAATTACCATCATTTCCTTCTTTGACTAGC | | GTCTATGCTGAGGTAATTCAAACCACAAATTGAGATCG[T/C]T | | | | | |
| CTAGGGAGAAGGTCTTTAAAGTATA_NODE_1_length_276_cov_ | | ATTATGTAAAGATTATATCATATTATCAACATAAACATAACA | | | | | |
| 53.155796 | | GCCGCTTTTAACAGGTTGGAAGGTACACGAGCCCTGA | | | | | |
| RAD_kmer_0003352_1_TGCAGGCATAATTTTACCGGTTCCC | 410 | TAAGGTGAAAGTACTGTTTTTTTTTTTTAATCATAGATTTTAA | T | T | T | A | GYSNP0981 |
| ACAAGAGTGATTGGAACCACTGGAACTCCAGTTTTCGCAG | | ATATGGAAAATTAAATAAAGGTGTTGTTTATTGTTTC[T/A]TT | | | | | |
| CAATACTGAATGCACCTTTCTACAAA_NODE_1_length_474_ | | GTAGAAAGGTGCATTCAGTATTGCTGCGAAAACTGGAGTTCC | | | | | |
| cov_87.622360 | | AGTGGTTCCAATCACTCTTGTGGGAACCGGTAAAAT | | | | | |
| RAD_kmer_0003361_1_TGCAGGTGTTTATGAGCTTATACCT | 90 | TACATAGGGCTTAGTCTTTTCAATAAAATTTAAGCCTTCCATA | T | A | A | A | GYSNP0982 |
| TATTATAAGGGTGAGAATACAGTTTTTGATGTTTCCCCTCC | | AAATTCTCTTAAGGGCTAAGGCCGGTATCTTCAGCAT[A/T]AT | | | | | |
| CTTTGTCACTGTGACTGTTGAGCAT_NODE_1_length_387_cov_ | | ACACACGCGCGCGCGCATATATGTATGTATACATACACAATG | | | | | |
| 38.330750 | | TATGTGTGTGCGAATGTGCGTATGCATATGTATTAC | | | | | |
| RAD_kmer_0003365_1_TGCAGGCATCACATGTTTACAGCGG | 260 | GATGTAGTAGTATTCCAATCTACCTCTATTTATATTCCTATCT | K | T | K | G | GYSNP0983 |
| ATGGGTGTTGGGTTCGCTATAAACATTTGTGCAACATTGG | | GGCAGCCAATTTTTCCCATTTTTTTCTGAACTATACT[G/T]ATG | | | | | |
| TGGGGTCGTTTGTAGAAATCAAAAGA_NODE_1_length_568_cov_ | | GACCAATGTCACCATTAATGTCCCTGCATACTGTCCGAGTGC | | | | | |
| cov_130.919022 | | AATTGCAATCCAATATAAAGCCATTGCTGTGCTTT | | | | | |
| RAD_kmer_0003365_1_TGCAGGCATCACATGTTTACAGCGG | 497 | CGAATACTGAGGAACCAACCAATACACGCTTATGGGTATTAT | Y | C | Y | T | GYSNP0984 |
| ATGGGTGTTGGGTTCGCTATAAACATTTGTGCAACATTGG | | ATGGGTTGGTTGGTCGATCAGATTATGATCTCTTGCTA[T/C]TT | | | | | |
| TGGGGTCGTTTGTAGAAATCAAAAGA_NODE_1_length_568_ | | GTTTTCTTTTGATTTGTACAAACGACCCGACCAATGTTGCACA | | | | | |
| cov_130.919022 | | AATGTTTATAGCGAACCCAACACCCATCCGCTGTA | | | | | |
| RAD_kmer_0003372_1_TGCAGGCGCTCTGTGTGTGTGTGAG | 133 | CTCCACTACAATACAGGGAGACAACCGAATCAAATTAAGCTT | T | Y | T | Y | GYSNP0985 |
| AGAGTTAGGCAAGAAGATAAAGGGTTACAAACTTACACA | | CATACTCTCATCTCTCATTAGTCATTATCGCCCCCCAT[T/C]TC | | | | | |
| GAGTAATCAAATAGGGCTTATTGAGAG_NODE_1_length_500_ | | CTGTCTGCCTTCGATATTTCAAACGATAGTGCTGGCTGCCTGT | | | | | |
| cov_94.444000 | | CTTTGCTCTCAATAAGCCCTATTTGATTACTCTGT | | | | | |
| RAD_kmer_0003383_1_TGCAGGCACACCCAACCACTCCAGG | 276 | GGAATGTGTGGACTGTCAGCGATTAGGTGAAAATTAATGGAC | A | A | R | G | GYSNP0986 |
| GCAAAGTTCAGTTATAAACACAATAACTTATAAATAATGA | | CGATTAGATGAAAATTACTCGGTGAACCTGTGAATAGC[A/G] | | | | | |
| GTAAATTCCACAAAATCAACACCGCC_NODE_2_length_342_ | | ACTAATCATAGGCTAGGCGTGATTTTTACAACCGTGGTATGG | | | | | |
| cov_36.663742 | | TGTTTAGTTATTACGCATGACGGCATTAGGTCATTTTC | | | | | |
| RAD_kmer_0003388_1_TGCAGGTCCTCCACACGGCTTTTCG | 367 | TACTCTCCACTCACAAAGGTAATCTTTTCATCAATTCCCTCAT | A | T | A | A | GYSNP0987 |
| CCCTATCCTTGAACACCTGGTGGGACCCAGACTTGCATTT | | ACCGTTTTATTACAGTTCCGTTACTCTATTAAATTAA[A/T]TAA | | | | | |
| TGTACCTTTCCCCATTAAAAACAACA_NODE_1_length_618_ | | CTTTGTTAATTTTTTGTGTATTAATTCTATATATCGGTCTATA | | | | | |
| cov_77.352753 | | TTTGTTGACTATAAATCAATTATTCTTAGCTAG | | | | | |
| RAD_kmer_0003392_1_TGCAGGTATGGTCAGGTATGGAACC | 160 | ATGGTTCCATGAGAATAGGCTGCAAGGGATCCACTTGAGCTT | K | G | G | — | GYSNP0988 |
| GGGTACACGATACCCAGTTTCAGTTTTTTTGGGTCCGCTC | | GAGCATATTGCTACTCCTAAACCGAACTTGATTGCTT[G/T]A | | | | | |
| ATACCCCGTTTTTTTGTGCACCTCTA_NODE_1_length_469_cov_ | | TTTTTTTTCATGATCTATGATTGATCTTGGCTCCAATCAATTCA | | | | | |
| 122.234543 | | AGATTGGTTCATACGACTTGAAAAGTTATATAAGA | | | | | |
| RAD_kmer_0003392_1_TGCAGGTATGGTCAGGTATGGAACC | 234 | TTGCTTGATTTTTTTTCATGATCTATGATTGATCTTGGCTCCAA | G | K | K | — | GYSNP0989 |
| GGGTACACGATACCCAGTTTCAGTTTTTTTGGGTCCGCTC | | TCAATTCAAGATTGGTTCATACGACTTGAAAAGTTA[T/G]ATA | | | | | |
| ATACCCCGTTTTTTTGTGCACCTCTA_NODE_1_length_469_cov_ | | AGAATTGGAATTAGGCTGGTCGAGCTTTTATGTTTCAAGCTTG | | | | | |
| 122.234543 | | TTGAATTAAAGCTTGACTTGAATGATTCAAGTCT | | | | | |
| RAD_kmer_0003392_1_TGCAGGTATGGTCAGGTATGGAACC | 354 | AAGCTTGTTGAATTAAAGCTTGACTTGAATGATTCAAGTCTG | S | G | G | — | GYSNP0990 |
| GGGTACACGATACCCAGTTTCAGTTTTTTTGGGTCCGCTC | | GCTTGAGCCCAATAACTTTATTAGATCCAGACTCATT[G/C] | | | | | |
| ATACCCCGTTTTTTTGTGCACCTCTA_NODE_1_length_469_cov_ | | AGCCTTAGGTATTATTGGAAGGTTGTTTAGCAAAGATGGGCA | | | | | |
| 122.234543 | | TCTTAGGTAGAGGTGCACAAAAAAACGGGGTATGAGC | | | | | |
| RAD_kmer_0003394_1_TGCAGGTAGGAATAACCTCGTCTTC | 223 | TAAAATATGGGGGGAGAATCGGTGAGGTGGAAATTCATGGT | A | A | A | C | GYSNP0991 |
| TGCATCTCTAGTCCACAACGACCACTCCAGCTGTACGGCT | | GAACGGGAGTACGTTGGGGCTTGTTGTGAAGCGAGCTTG[A/C] | | | | | |
| GAGATCGGATGAACAGCATGCGCTCT_NODE_1_length_619_ | | AGCGGCTTGATGTTGATTACATTGATTTATACTATGTGCATCG | | | | | |
| cov_154.092087 | | CATCGATACAAAAGTCCCTATCGAAATCACAGTTAGT | | | | | |
| RAD_kmer_0003399_1_TGCAGGTTAAACAAAGTGAATTTAT | 135 | GGAAGGTCAACCAATTTCTGGAGATAATCAACTGATTATTGT | Y | T | Y | Y | GYSNP0992 |
| AAAAACATTTTCAAACCAGCATTATGGGCGATATCTCTTA | | GCTTCTAATGTTCTTAAAATATGCACTGATAAAATTTT[T/C]TG | | | | | |
| ACTACTGTTAATAAAGAAATATTACA_NODE_1_length_644_ | | AGAAGTGGAGAAAATTTCTTGAGATTTGTGCTGTTGAAAAAT | | | | | |
| cov_161.461182 | | ATTAAAACATCAATTAGACATTTTTACTTTTAAGAC | | | | | |
| RAD_kmer_0003399_1_TGCAGGTTAAACAAAGTGAATTTAT | 498 | GCCACTGATTTTGAATTTATCCGTCAACAGACTGTTGTCACTT | R | G | A | A | GYSNP0993 |
| AAAAACATTTTCAAACCAGCATTATGGGCGATATCTCTTA | | CACACTTTACCGGTGTTTGCAGGACTTTAGATAGTTT[A/G]TT | | | | | |
| ACTACTGTTAATAAAGAAATATTACA_NODE_1_length_644_ | | AAGTTTAAAATGATAACTTTGTTTATGTTATGGACCAAGTCAC | | | | | |
| cov_161.461182 | | CAAACATGCAGATGAACGTTTACATAGTGTCTAAC | | | | | |
| RAD_kmer_0003405_1_TGCAGGGAGATATTATGGATGAAAT | 169 | CTACTGTGCACACTTCTGTCTCCTCTGGTTCAGTGACTTCAGT | A | — | A | T | GYSNP0994 |
| ATGATCCAAGGGCTAAAAAGTGGCTCCTGTTAATGGTTCT | | CACCTTATTGTGCTACAAGTGAAGACATACATGTTGG[A/T]CC | | | | | |
| CATTTGAACCTCCCTCTTGTTTGTAT_NODE_1_length_257_cov_ | | TGTTTTGGTATTATAAAATTATACAAACAAGAGGGAGGTTC | | | | | |
| 227.984436 | | AAATGAGAACCATTAACAGGAGCCACTTTTTAGCCC | | | | | |
| RAD_kmer_0003405_1_TGCAGGGAGATATTATGGATGAAAT | 85 | ATCATGATCATGATCATCATTATCAAGTGAACATGGTGG | T | T | C | — | GYSNP0995 |
| ATGATCCAAGGGCTAAAAAGTGGCTCCTGTTAATGGTTCT | | AACATCATCAACATTGTTAATATTGAGTGAGTCTTGAG[C/T]A | | | | | |
| CATTTGAACCTCCCTCTTGTTTGTAT_NODE_5_length_236_cov_ | | GGTGTAGCCTCATGAAGAATAAACTTTGCAGATTGCCTCCTT | | | | | |
| 10.478813 | | CTTTGGAGCCTTTTGGATTTACCCTTGTCATTATCTT | | | | | |
| RAD_kmer_0003407_1_TGCAGGTCCGGAGAGGATTGCTAGG | 539 | GATTTGGTTATCGGTACCTATATTTGACGGCGAGTTGTTGCAA | S | S | C | S | GYSNP0996 |
| CATGTCTGGTAATGAAAGTAGCCTTGCTACAATTCTTGGT | | CATCTAAAGGACACGTAGTGGCCCCTGCAAGGCTTCC[C/G]GC | | | | | |
| GCAAAATTTGCAGCGGCGTTTGTTGC_NODE_1_length_604_ | | AACAAACGCCGCTGCAAATTTTGCACCAAGAATTGTAGCAAG | | | | | |
| cov_165.399002 | | GCTACTTTCATTACCAGACATGCCTAGCAATCCTCT | | | | | |
| RAD_kmer_0003412_1_TGCAGGAATGAAGGTTCTATATTAT | 279 | TATGTAGGTAATTCGGTCTTTGAGATTTGTATGGGGTAGGATA | A | A | R | G | GYSNP0997 |
| ATCCTTGCACTATTTGTCTGATAACTACAATTACCAACAT | | TGTGATATATAATTATGCATAAACATAGATGATGTAT[G/A]AA | | | | | |
| AGAAGCATTGAAGTAATCATCAAGAA_NODE_1_length_567_ | | CCACAGGATTAAAAGCAATTTCCATTATCGAAAAGACTCCAA | | | | | |
| cov_75.410934 | | TTCTATTATACACTTTGGGCCCCTAAATACGTTTAG | | | | | |
| RAD_kmer_0003412_1_TGCAGGAATGAAGGTTCTATATTAT | 405 | TCTATTATACACTTTGGGCCCCTAAATACCTTTAGTCGGCCCT | G | G | G | A | GYSNP0998 |
| ATCCTTGCACTATTTGTCTGATAACTAGAATTACCAACAT | | CCACGTGTACGTAGATTAGGTTAAGCCATTTTTATAG[G/A]AT | | | | | |
| AGAAGCATTGAAGTAATCATCAAGAA_NODE_1_length_567_ | | TAAGGTTTGGAATTTGATGTACTGCAACCCGTTGAGTAATGTT | | | | | |
| cov_75.410934 | | TATTGTCGATGGGTTTGAACTAAATATTGGGTTTG | | | | | |
| RAD_kmer_0003418_1_TGCAGGTACAGAACCATTAGATGAC | 500 | ATGAGAGGATGGGTGGACCGACGCCTAATGTCTCTTCTCAAG | A | A | T | A | GYSNP0999 |
| AAGACATTTAATCCAGGTGAAGAGGACATAGGTGCCTGC | | TTCCTAGTAATGGCGTTTCTTCCAAATTAAATGGTCTT[T/A]CT | | | | | |
| AAATTGGACGTTCCTGCAACAGGGGTG_NODE_1_length_602_ | | TTTGCTGTTAGTGGGACTGGAGCTAAAGATTCTAGCACCCCT | | | | | |
| cov_109.559799 | | GTTGCAGGAACGTCCAATTTGCAGGCACCTATGTCC | | | | | |
| RAD_kmer_0003422_1_TGCAGGTAGATTATGATATTCTCAA | 97 | GATGTGGATGAATCATTAATATACTACGATTTTGGTATGATG | T | T | Y | — | GYSNP1000 |
| GACTAATAACAAATCCAGATAATATATAAGTATATATGTA | | GGGGAGATCAAGTCATTAACAAGAGAAAGATTGATGGA[T/C] | | | | | |
| AAAAGGTACTTTTAAGTGTGAATAAA_NODE_1_length_617_ | | CTTTTTTATGCTGTTTATGAAAAAGATGCAAAAAAGGTTTTAA | | | | | |
| cov_91.215561 | | CTTCCGTAGTTTCCTCGCCTTTCTTATACCATAAAGT | | | | | |
| RAD_kmer_0003425_1_TGCAGGAGGTAAGCTCCTGGATATG | 159 | CGGCAAGATTAGCTTACTTGCAGTTTAGGCCGAAGCGCCTCA | G | G | A | A | GYSNP1001 |
| TTCATATGTCATATTTCTTGAAATCCTACTATCCTTTTTTTT | | AGAGGTCCTTTGGGCTTCTCTACACCTTGCTGTTAACA[A/G]T | | | | | |
| TTATTTATTTTTTATTTTTTTATA_NODE_1_length_604_cov_86.132454 | | AACATTATGTTTTGGGTCAACTAATATACAACTTATATTACGG | | | | | |
| | | TAAAAGTTTTAAAATAGCAGTCAGCAAATTCTCATG | | | | | |
| RAD_kmer_0003425_1_TGCAGGAGGTAAGCTCCTGGATATG | 480 | CTCGTGAATGGATTTATAAATCTTACCTTAGAAATAAGTGGC | T | T | T | G | GYSNP1002 |
| TTCATATGTCATATTTCTTGAAATCCTACTATCCTTTTTTTT | | GGCTTTTTTGGCACAATACCTCCATACTTCTTCTTGAT[T/G]AC | | | | | |
| TTATTTATTTTTTATTTTTTTATA_NODE_1_length_604_cov_86.132454 | | TTCCTCCTAAGAAAGCACAATGTAACAAAAGCAGTGATTATT | | | | | |
| | | ATTTTGACACACTAGTATAAAAAAATAAAAAATAAA | | | | | |
| RAD_kmer_0003429_1_TGCAGGAAGATCTTATTGCTCCCTT | 232 | CTTTTGTTTTCAATCATACCACGATCTCTTTGTTGTTGAAAAT | T | C | Y | — | GYSNP1003 |
| AGAACAGCTTGTCAATCTCTTCTTGACCAACAAGGTCAGT | | ATGAGTTTTCAACATCCTCCATTCGAATCTTTTTCTT[C/T]GGT | | | | | |
| ACATTCATGTTTTTGAAAACCCTAAA_NODE_2_length_409_ | | CGATTATTACTTAACGACTCATTAGGTTCGTTTAGTTTTTCGA | | | | | |
| cov_56.185818 | | CATTGGAACTATATGATTCCATTAATCTCTCCAA | | | | | |
| RAD_kmer_0003435_1_TGCAGGGAAACCGCGTTATAAACA | 370 | ACCGTTTTCGCTCATGATTAGTTGTCTTAACTCTTAACTAGAA | C | Y | Y | Y | GYSNP1004 |
| ATGAAAACAAAATCTCTATCTTCCATATTCTAAGTGCTCA | | TATTCTTGTCAAAGGTATTTGCATTACAGAATGTAAC[T/C]CA | | | | | |
| ATTTCATTATAATTATAAAAATGGTTA_NODE_1_length_642_ | | TGCATGTATTTATAGATGCAAGTCATTAATATATATTTTAAGT | | | | | |
| cov_81.593460 | | TGTTGAAGGCAGGTTCCAAGCAGCTTCAATCTACT | | | | | |
| RAD_kmer_0003435_1_TGCAGGGAAACCGCGTTATAAACA | 515 | GCAGCTTCAATCTACTTTCAAGAATAGATTTTTATGTTTTAAT | G | R | R | R | GYSNP1005 |
| ATGAAAACAAAATCTCTATCTTCCATATTCTAAGTGCTCA | | GTTTGCAACAAGACAATGATCTTGGTTTTGACCATTG[A/G]TA | | | | | |
| ATTTCATTATAATTATAAAAATGGTTA_NODE_1_length_642_ | | GACTTGACATGTGTCCCTTCACTCACTCTGTGTGTTTTCTATTC | | | | | |
| cov_81.593460 | | ATGGGATCTTTGTAATTAACCATTTTTATAATTA | | | | | |
| RAD_kmer_0003437_1_TGCAGGACAGGTTAGGCTACTTGTC | 153 | AATCAGAGTATCAAACAAACCAAAAGTCACGACTAAACAAT | W | W | W | T | GYSNP1006 |
| AAGGATATTTTGATTTTGTTATCAAGAAGCTAAGTATCTA | | CAGAACTCAGAAGTTGTAATCACATTAATATTTTGTGCT[A/T] | | | | | |
| CTGTCATGGCAAACTAAGACGTTAAG_NODE_1_length_617_ | | AACCTCTAAAGTGATGAATTTAAATAAGCAACCCCACTCCTT | | | | | |
| cov_136.529984 | | ATGGTATGTGAAAAAAGATGTTTTTCCTTCTAGTCAAC | | | | | |
| RAD_kmer_0003445_1_TGCAGGAACTTTGACTTTTTCGCGT | 296 | ACTACCTTCCCCATATCTCCAAATAAAATAACACGTTGCAAT | G | C | C | C | GYSNP1007 |
| AGTAACATGTGTGGTATGACATCACGTTTTAACCACATAA | | GAATCTTGTCCAGGATATGGAGATGACTTAAAAGAATA[C/G] | | | | | |
| AAATTTGTTAAAAGATAAATGGAGAT_NODE_1_length_601_ | | CTTCTACTCCAAACAATAGATCCATTCACCAACCTATGACCC | | | | | |
| cov_177.470886 | | ATCCTGTAATCGTACCTGCAGCAGAATGTTAAATTTTT | | | | | |
| RAD_kmer_0003445_1_TGCAGGAACTTTGACTTTTTCGCGT | 448 | TAAATTTTTAAAGAACATATCATTTAAGAAGGAAAACTGATA | A | C | C | C | GYSNP1008 |
| AGTAACATGTGTGGTATGACATCACGTTTTAACCACATAA | | TAAATCGTACATGGTGTTAGGCCACAAATTATTCAAGA[C/A]A | | | | | |
| AAATTTGTTAAAAGATAAATGGAGAT_NODE_1_length_601_ | | CTAGTGTGTATAAAACCAGGATCTCGCCATCCGACAGTACTT | | | | | |
| cov_177.470886 | | GCAGGTGGACCTGCAAGCGAATAAAACTTAATATATT | | | | | |
| RAD_kmer_0003445_1_TGCAGGAACTTTGACTTTTTCGCGT | 504 | TGTTAGGCCACAAATTATTCAAGACACTAGTGTGTATAAAAC | A | G | G | G | GYSNP1009 |
| AGTAACATGTGTGGTATGACATCACGTTTTAACCACATAA | | CAGGATCTCGCCATCCGACAGTACTTGCAGGTGGACCT[G/A]C | | | | | |
| AAATTTGTTAAAAGATAAATGGAGAT_NODE_1_length_601_ | | AAGCGAATAAAACTTAATATATTTATTAAGAATCTCCATTTAT | | | | | |
| cov_177.470886 | | CTTTTAACAAATTTTTATGTGGTTAAAACGTGATGT | | | | | |
| RAD_kmer_0003446_1_TGCAGGCACAGCATAACTTGATCAA | 105 | TCAGAAAAGAAAATAAAAAGAATGATTAAACAATAAAACGT | R | G | G | R | GYSNP1010 |
| AGAAGTTAAAATAGAATCAATCCACACCTTTGTTTCTCTA | | ATACTAACTAAAAACGTATATAAAATAAGCACACGACAA[G/ | | | | | |
| AAACCGTTGAAACTTCAAACGTGCAT_NODE_7_length_323_ | | A]TTTAGGCCAAAACGTAAACCAACAAAAAAAAGTGTTTATG | | | | | |
| cov_40.916409 | | TCCAAATATAACCAACTTGAATTTATAACAATAGATAAAA | | | | | |
| RAD_kmer_0003446_1_TGCAGGCACAGCATAACTTGATCAA | 230 | AATATAACCAACTTGAATTTATAACAATAGATAAAAAAAATA | — | C | T | T | GYSNP1011 |
| AGAAGTTAAAATAGAATCAATCCACACCTTTGTTTCTCTA | | AATACGTAGGTGTCAAAATAAAAATTTTTAAAAGTGTA[T/C]A | | | | | |
| AAACCGTTGAAACTTCAAACGTGCAT_NODE_7_length_323_ | | AAAGTTGAGGGGGTGATGTTGAAAAGAAAAAAGAAACGTAT | | | | | |
| cov_40.916409 | | TGTTCCATTAGAATGAACCGAATTAAAATTGAGGAGGT | | | | | |
| RAD_kmer_0003450_1_TGCAGGCCCAGGCCCATTGCATC | 216 | GTCTGAATGGTTATCTCTTAACCATTCAGACTTAAGGTTGCCT | C | A | A | A | GYSNP1012 |
| CGGTCTTTAAATTCACAAGTTTCATACTTAACTGGGCTTCT | | CTTAACCAATAAGAGGCTATGAACCATTAAGAGGTAG[A/C]C | | | | | |
| CTCACTATGTCTGTGTAATTTCCTG_NODE_3_length_402_cov_ | | TATTAATGACCATTCAGAGGCTACCAAACAGCCCCTATTTCGT | | | | | |
| 43.149254 | | TGCATAGTTGCGAAAAGTATGTCCAACGAGCCCCAAG | | | | | |
| RAD_kmer_0003454_1_TGCAGGATCAAGATAGTTTAGAATC | 535 | TTTTTCTGCATAAAATTTTCGATGCAGCACCTATTGAGTAAAT | G | — | S | G | GYSNP1013 |
| AGAATCATACAAGATCAAGAACTACCCTGTATGACAGAC | | ACTTGATATTATTTGTATTGACGAAGTATTGTACACG[C/G]AC | | | | | |
| ATTGTCCTGATACGACAATTACTTTTT_NODE_1_length_627_ | | TTCACTATTTTCAAAAGGTTGTATTAAAAAGTAATTGTCGTAT | | | | | |
| cov_116.503990 | | CAGGACAATGTCTGTCATACAGGGTAGTTCTTGAT | | | | | |
| RAD_kmer_0003460_1_TGCAGGAGGAAAGAAAAGAAAACA | 170 | AATTAACACGTTACACAATTAACTATAATTCACACAAAATGT | G | R | R | A | GYSNP1014 |
| ATCGGATGTGCCGAATTCCAAAGCCCCAATTGATGACAAA | | CATGAGATACATGCATGATTAGTTTTCAAAAGGATAAT[A/G]C | | | | | |
| CGGGCTAAAAAATTACCCGAGCAGCCT_NODE_1_length_648_ | | ATTGCTAGCCTAAGTTACCTGTCTCTTTCTCCTCTCACCGATT | | | | | |
| cov_79.841049 | | GAAAGTCAACAGCAACAGAAGTATAGTAACGCTAAA | | | | | |
| RAD_kmer_0003460_1_TGCAGGAGGAAAGAAAAGAAAACA | 506 | GAACCTTTACCAGATATTGAGCCATTTTACTGAACCGAAACA | K | G | K | G | GYSNP1015 |
| ATCGGATGTGCCGAATTCCAAAGCCCCAATTGATGACAAA | | AAAACTTTGACCGGTGGCTCCTCAATGACATAATTAGG[G/T]T | | | | | |
| CGGGCTAAAAAATTACCCGAGCAGCCT_NODE_1_length_648_ | | ATCAAACTACTTTTGAACTGTCATTGATGCCATCATTCCCGTC | | | | | |
| cov_79.841049 | | ATCAAGCATAACGATGACATCACCATCGTTTTCAGG | | | | | |
| RAD_kmer_0003463_1_TGCAGGTTGCTTGGGACACCAACTG | 216 | AACACACATACACAAAGCACTAAAATTGTTAAACAAAACAC | C | T | C | C | GYSNP1016 |
| AAGAGCAGTGGCCAGGGGTGAGGTCTCTAAACAACTGGC | | ATAGTAAAATCACCAAAGTGATGATGAAATTTTTTTAAT[C/T] | | | | | |
| ATGCCTATCCAAGGTGGGTACCTCAAA_NODE_1_length_520_ | | AGACAGCAGGTGAAGCTAAGCTTTAGAACTGTGATTTGTCAA | | | | | |
| cov_88.259613 | | GACTATCAAAATAAGGATGATCCATAGCAGCTTTGGCT | | | | | |
| RAD_kmer_0003466_1_TGCAGGATTGTTTTACATTAAACAC | 371 | AAGCTATAAAAATTGTCCAAATTGTCCTTAACTCCTTATGTGA | A | T | T | T | GYSNP1017 |
| ACTTTTGTTTCCCTGGGTTTGTAAAATATTATGAAATTCCT | | CATAATTACACTGTATGTCCCTGCTTTTACTGTTCCT[T/A]TAT | | | | | |
| GATGTGACATAATTACACTGCATGT_NODE_1_length_501_cov_ | | GGTTTGGGTCCCTAGTCATCTGAGTAAAATGACTAATCCATA | | | | | |
| 119.167664 | | ACAGAACAGGAAAAGAAAGGACATGCAGTGTAATT | | | | | |
| RAD_kmer_0003466_1_TGCAGGATTGTTTTACATTAAACAC | 432 | CCCTGCTTTTACTGTTCCTTTATGGTTTGGGTCCCTAGTCATCT | C | A | A | A | GYSNP1018 |
| ACTTTTGTTTCCCTGGGTTTGTAAAATATTATGAAATTCCT | | GAGTAAAATGACTAATCCATAACAGAACAGGAAAAG[A/C]AA | | | | | |
| GATGTGACATAATTACACTGCATGT_NODE_1_length_501_cov_ | | GGACATGCAGTGTAATTATGTCACATCAGGAATTTCATAATA | | | | | |
| 119.167664 | | TTTTACAAACCCAGGGAAACAAAAGTGTGTTTAATG | | | | | |
| RAD_kmer_0003467_1_TGCAGGCGCAAGGGGCGTTGCCTCC | 336 | AACGTTTTTATGACTATTTTTCTTTTTTTGATCGTTTTTTTTTTG | G | K | G | — | GYSNP1019 |
| TTGGAACCCCTGATGTATCTTGGTGTCGGATGTCTTATTAG | | TTTTTATACAAGATTTTTCGGCGTTGGTGATCAGC[G/T]GCCGT | | | | | |
| TTTTGAACGAGCATGTACGTAACAC_NODE_1_length_414_cov_ | | AGTGTGACATTGGTCTCATAGATTTTCCGTTAAAGGATTATCT | | | | | |
| 34.137680 | | TGATTTTGGCTAGAGCTCCATTATTGTAAAGG | | | | | |
| RAD_kmer_0003469_1_TGCAGGTTTGACCATCTTTTGCTCG | 582 | GAAAATTCCATTATAGCTAACAAAATTCATCCAACTTTTAGTC | — | T | T | C | GYSNP1020 |
| AATTTCTTAATATTCGTTTATTATTAAGTATGAACTTTTGT | | AAAACGTCTATGATCCGTTCTTCACGGCCAATTAATT[T/C]AA | | | | | |
| TGTGAATACACTAGTTACTTAAATT_NODE_2_length_642_cov_ | | GTAACTAGTGTATTCACAACAAAAGTTCATACTTAATAATAA | | | | | |
| 90.517136 | | ACGAATATTAAGAAATTCGAGCAAAAGATGGTCAAA | | | | | |
| RAD_kmer_0003474_1_TGCAGGCAGCCTTAACTTCTGGCAG | 285 | GAGCACTTTATAGGGACTTATTGGGGTGCGGTCGGGACATCA | Y | G | Y | C | GYSNP1021 |
| TTAAACCATGAATCTCCTGTTTCAGCAAGAACTGCAGTGT | | TTTTGTTCCGAAATTGTTGAGTCTGCCGACGCTTATCT[C/T]TT | | | | | |
| TCTCATTGAGCATTTTCTGTATATGT_NODE_1_length_622_cov_ | | TGCTGGGCCCATATTCAACGACTATAGTTCGGTCGGCTACTC | | | | | |
| 157.657562 | | ATTGTTGCTCAAGAAAGAAAAAGCCATCATTGTGCA | | | | | |
| RAD_kmer_0003484_1_TGCAGGTAATCATCGATCGAAAAAT | 391 | GAAATAAGAGGTATGAGAATCAGCAAAACAACAACGGCTAA | C | — | Y | C | GYSNP1022 |
| GAGTACTGCTTTATATTTTTATATTACATCTTATCATATAG | | ATCCTGACAAAAGAATTAATAAATAATAAACGTGTTAAA[T/C] | | | | | |
| CTTTACATGTTGAACCTGTATTGCA_NODE_1_length_611_cov_ | | GCAATGTTTTCTTGGAACTTTTTAAAAATCATATTATATCCAC | | | | | |
| 113.842880 | | ACAAGCACCCATAAACTTATATTCATGATTGGATGTA | | | | | |
| RAD_kmer_0003485_1_TGCAGGGACAAAGGGAGGATCGCT | 431 | TTTATTTAGAAACTCTGGTACGCTTGAACCCTCATAATATAAA | Y | Y | T | T | GYSNP1023 |
| CTTTCGCAATACATGAAAACAATCAACCTGAAAAGCAAG | | CTTTCAATTTTTTGTGTTACCCAAAAATATTTTGAAG[T/C]TAT | | | | | |
| ATCAAAGGGTTGACTACTGCATCAAATC_NODE_1_length_510_ | | AAAGCCCTCTTGATTTGATGCAGTAGTCAACCCTTTGATCTTG | | | | | |
| cov_112.747055 | | CTTTTCAGGTTGATTGTTTTCATGTATTGCGAAA | | | | | |
| RAD_kmer_0003493_1_TGCAGGCTACCATCAAGTCTAACCA | 276 | TCATTAAGCACGTGATTGGTTATCGTAACCAGATCATTAAAT | C | T | T | T | GYSNP1024 |
| GCTTATCATCCGTTCACAGAAACGTGAGGAAACCACAACT | | ACGTGACGGGTATTGTAAGTAGCAGAGTGCCTTAAAAG[T/C]T | | | | | |
| GCTGACGCAGCAATCTCTCGCCGTTT_NODE_1_length_559_ | | AAAACATCTAATATATATTTTTAAACTTTGAATTCTAGTTATT | | | | | |
| cov_157.296951 | | TCCCTCACCAAAAGTGAGGGCGTATTGAAAGTAGAT | | | | | |
| RAD_kmer_0003493_1_TGCAGGCTACCATCAAGTCTAACCA | 385 | TTGAATTCTAGTTATTTCCCTCACCAAAAGTGAGGGCGTATTG | C | A | C | C | GYSNP1025 |
| GCTTATCATCCGTTCACAGAAACGTGAGGAAACCACAACT | | AAAGTAGATAATTAAATATGTGACGGGGTATTGTAAC[C/A]A | | | | | |
| GCTGACGCAGCAATCTCTCGCCGTTT_NODE_1_length_559_ | | GCAGAGTGACCTTGAAAATAAATAGAGTTACCTGCTTCACCA | | | | | |
| cov_157.296951 | | TAAGCGGCATCTGCGGGAGAAACCTTGGTGCCAACAG | | | | | |
| RAD_kmer_0003495_1_TGCAGGTATCTGTTTGAGTTTTGATC | 237 | GACCCCTTGTTTGTGCAAACTACAACAAAATGGACCTTTAGC | G | — | G | R | GYSNP1026 |
| ACGTGATACTGTCAACAATTACAAACCATTTTATGAAGCA | | GATGACAATTGTCGCCGCTATTGCCTAGAATTGTCGTC[G/A]C | | | | | |
| TAAAATTTTCTAAAGAAAAGGTCAT_NODE_1_length_569_cov_ | | TAAACATTTAGCGGCGACATATCTCCGCTAAAGGCCGTCGCC | | | | | |
| 80.434097 | | GCTAAAAGTTTGTCACTAAAGGCCAAGCTGTCACCGC | | | | | |
| RAD_kmer_0003500_1_TGCAGGAACATTTGGGTATATTGAT | 110 | ACAAGTTCACTTATTTTGTTATTCTCATAGTACATTCGTACCT | T | T | T | C | GYSNP1027 |
| CCGATGTACCAAGAGACAGGGGTACTAACGAAAGAGTCA | | GTCCTACTAAAGGCTGGTGATTTTGGTTGCCATAGCA[T/C]AA | | | | | |
| GATGTCTACTCTTTTGGCGTAGTGTTG_NODE_1_length_198_ | | CCTCCCACATAACACTTCAAACAACACTACGCCAAAAGAGTA | | | | | |
| cov_331.338379 | | GACATCTGACTCTTTCGTTAGTACCCCTGTCTCTTG | | | | | |
| RAD_kmer_0003500_1_TGCAGGAACATTTGGGTATATTGAT | 144 | TTTTCGGAATGAAAACAGGGGATTGTTATTCGGATGTCTTCTT | A | G | G | G | GYSNP1028 |
| CCGATGTACCAAGAGACAGGGGTACTAACGAAAGAGTCA | | CTTCGTGAACGGTGGAATGATTGGGAACCAATGCTCT[G/A]AT | | | | | |
| GATGTCTACTCTTTTGGCGTAGTGTTG_NODE_2_length_361_ | | GCCAATTGTGAGATCACAGGATCGATGAGGGTAAAGGATTTG | | | | | |
| cov_34.817173 | | AGAGAGAATTCTTATAAAACAAAGATTCAATACATT | | | | | |
| RAD_kmer_0003500_1_TGCAGGAACATTTGGGTATATTGAT | 289 | AAAGATTCAATACATTTACAAATGATACAATATGTTTGGTAT | C | T | T | T | GYSNP1029 |
| CCGATGTACCAAGAGACAGGGGTACTAACGAAAGAGTCA | | TTATACAAGTGAAAGGTTGTGTCTCTTGGACATAACCG[T/C]C | | | | | |
| GATGTCTACTCTTTTGGCGTAGTGTTG_NODE_2_length_361_ | | GTGATGGTTGGTTGTATAACCGCCACTAACTAACCAACTAAT | | | | | |
| cov_34.817173 | | ATATACTTAGGATAAGTATCTAACTTAGGATAAAAGG | | | | | |
| RAD_kmer_0003503_1_TGCAGGAGATCATATAATGTTCTGA | 136 | GACAGATTGCTCACCTCCATATCAATCCTAGGATAAGTATAA | T | T | Y | C | GYSNP1030 |
| GTGACTACTTTGAGAGGAAGTTACTTTGTAGCCAACAGTT | | GAAAAGACAATCTCCTCAATGCACCTACGAAGTCCATG[T/C]G | | | | | |
| ACTTTCAGATGAGAGATATGAGAATA_NODE_2_length_353_ | | TCTGACAAGTGGAAGGAAAACAATTCAAAGGATTACATAGA | | | | | |
| cov_48.977337 | | TGCATATAGGACTGAATCAAATTCATTGACATTTATCA | | | | | |
| RAD_kmer_0003507_1_TGCAGGTGGCCTATCTTAGCTCTTCT | 238 | GCCATAACCGGTTAGAGTTTCAAAATAACCAATGGTTTGTAG | T | A | W | A | GYSNP1031 |
| TGGGCCCACTTAATCTTTTCCTAGGACCAAAACTTTTTTTT | | CCGGTTAGTGTATTTTGGGGGAGGAACCGGTTATTTTT[A/T]A | | | | | |
| TCTTTAGGTATTCAAAGAATATTC_NODE_1_length_529_cov_ | | CCGATGTATCGGTTAACCGGGAAAAAAACCAAACAATTGAA | | | | | |
| 82.160683 | | GAAAAAAAAAAACTGAAAATAACACGTTAATTAACCGA | | | | | |
| RAD_kmer_0003510_1_TGCAGGCAATTGGGGACGGTGTCGC | 252 | ACATTCAGATTATTATCGTAATGATATTACGTTTTGTAATCAT | A | C | A | — | GYSNP1032 |
| CCTTATGGGGGTGGTTCAAATGGTGATTTTTGGAGACGGT | | AAATAATGCATCAACTATTTAAGCAAAAACAATATTA[A/C]A | | | | | |
| GTTGCCCTTACAGGGGCGGTTCAAAT_NODE_1_length_606_ | | ACAGAGAAGAAAGGGATTGCATGTCAAACCAAAACCTGCCT | | | | | |
| cov_100.033005 | | TATTTGGACTCGTCCTACAATATTTGACATTTCAAGAC | | | | | |
| RAD_kmer_0003511_1_TGCAGGCCAGATCATTAGACAATTA | 89 | ACATGCTGAACAACTAACGCGACATTCGCTTTAATGTATTAA | C | T | Y | T | GYSNP1033 |
| GGCAATTATGTTATTTAAGAAACATCATACAAATTGAAGT | | CCTTTCGTTTTAAAATGTATAAAATTATTATACTATTG[T/C]TC | | | | | |
| GAACAGAGGGACAATCAACCTGAACA_NODE_1_length_481_ | | ACTGAAGTTGTATGGACGAAAGGTGACTCAATTTCTAAAAGA | | | | | |
| cov_198.066528 | | ATTTGGGAGCATTGAATCAGGGCAACTCATAGCTTA | | | | | |
| RAD_kmer_0003514_1_TGCAGGATCGCTCACTGTCTTCTTC | 206 | CATGGGAAATGGGTTCTAACATAAAACAAGCATTGGGCTACG | A | T | T | T | GYSNP1034 |
| GTGCTCGCCATATTGCTTACTTGCGCTGTTTATGATTGTTT | | AGTGGGGTTATGAGGTCCATATGGAGCGGGCGGAAGGG[T/A] | | | | | |
| GATCATCCCCTTTTGGAAGAAATGG_NODE_1_length_626_cov_ | | CTCAACCTGTCGGATCATGTGGAAAACAACCATTGCATGTTT | | | | | |
| 137.632584 | | TGCTAATTAGCTACGAGACCCGAATTCCATCTATTAGG | | | | | |
| RAD_kmer_0003516_1_TGCAGGTCAATATTGTTAGTTTACA | 262 | GCGACCCAGTCTGCTAACAAGTAAGCACACCATATGAATATT | R | A | R | A | GYSNP1035 |
| TCTAACTTCCAACTCTTTCATCACCAGCTTCACTCCAACAT | | CTGATCCTTCTTTTGGCGATATACTTTCTTCGGTGACC[A/G]CT | | | | | |
| ATTCATCTTTTACAGTCCAGAAATT_NODE_1_length_573_cov_ | | GTAGATTAGTGAAGTTTGTAGAGTGAGGCTGAGAAGAACCA | | | | | |
| 117.916229 | | ATGCTCTAAGCTCCCATTTGTTCCATTGTTTCCTCAA | | | | | |
| RAD_kmer_0003517_1_TGCAGGACCGCGGCCAAAGCCAATT | 110 | AATAAAATGGTGTGTATTGGTTGCTGTAGGTTATTAGTATGTT | C | C | C | A | GYSNP1036 |
| CATTTTCATAATCACCTCCAGTTACAAAGAAATGCAAGTC | | TGGGAACTATTGTATTGCTGCTAACTGCTATTCATAG[C/A]TT | | | | | |
| TTGTATGAACTTATACACCACAACAT_NODE_1_length_584_ | | GTTGATAGGCAGTTATAAACTGAAATAAAACTTAGAATTATA | | | | | |
| cov_181.839035 | | TTCCTGACTGAGTGTGTTATAGGATGCTTGTCCTAC | | | | | |
| RAD_kmer_0003517_1_TGCAGGACCGCGGCCAAAGCCAATT | 362 | GCTTTTGAAAGGTCCATCTTTACTAAAACACAGAAGACTAAT | C | C | C | T | GYSNP1037 |
| CATTTTCATAATCACCTCCAGTTACAAAGAAATGCAAGTC | | GCAAGGACTGAAGGTATTATTATAATTGACTTTTATCT[C/T]T | | | | | |
| TTGTATGAACTTATACACCACAACAT_NODE_1_length_584_ | | CACACTTACTGCTAAGGTATATATCGAATACCTTATTATGAA | | | | | |
| cov_181.839035 | | GATAGTTGTGCCTGCAGCTGGAATGCTTTCAAAGATA | | | | | |
| RAD_kmer_0003525_1_TGCAGGAGAGTAAAATGAATTCTTT | 519 | TCACCCAGAAGTACGTGCCCTCGGGTTTCTTCCCAAAGAGGG | T | G | K | K | GYSNP1038 |
| AGCCTTCAACACATCAACCCATACCATGCCTGATAATGAT | | CCATGGTGTATATGTCGCAAGGTATGGCTTTGACCCGT[T/G]T | | | | | |
| GCTGGCTGTGCAAGTCTAGACTGCCA_NODE_1_length_594_ | | TTTGCAAGATGGCAGTCTAGACTTGCACAGCCAGCATCATTA | | | | | |
| cov_161.538727 | | TCAGGCATGGTATGGGTTGATGTGTTGAAGGCTAAAG | | | | | |
| RAD_kmer_0003527_1_TGCAGGCGATTTTAGTTTACTTATTG | 432 | AAGGATGAAATCATTCAAATCATGACACTGACAAAGATCTAA | T | — | G | G | GYSNP1039 |
| GTGACTGGTACAAGTCCAACCATAAGGTGAGGATCATGTT | | TAATAATTTATATCATGTTTGACTTTCAAGAGTCAAAA[G/T]T | | | | | |
| CAAAGTCATAGTCAAATTAATCTTA_NODE_1_length_632_cov_ | | ATTGGTTTCTATCCTCAAAATCAACAAGATTTATAGAAAAAA | | | | | |
| 125.781647 | | TTTCAAAACCTTTACAACATTTAACAAAACAAAATCA | | | | | |
| RAD_kmer_0003527_1_TGCAGGCGATTTTAGTTTACTTATTG | 487 | CATGTTTGACTTTCAAGAGTCAAAAGTATTGGTTTCTATCCTC | T | — | Y | C | GYSNP1040 |
| GTGAGTGGTACAAGTCCAACCATAAGGTGAGGATCATGTT | | AAAATCAACAAGATTTATAGAAAAAATTTGAAAACCT[T/C]TA | | | | | |
| CAAAGTCATAGTCAAATTAATCTTA_NODE_1_length_632_cov_ | | CAACATTTAACAAAACAAAATCATTTACTTGATCTCAAAATC | | | | | |
| 125.781647 | | CAAAAACAATAACTTCAAAATATTATATGAAGACTC | | | | | |
| RAD_kmer_0003528_1_TGCAGGTTTGATTTCAATAACTTGT | 525 | CTTGCAGCATAGCTCTTGGTTAAACCTATTTACATTCAGATAC | G | G | S | S | GYSNP1041 |
| AAGTACAGTATTCAAGAAATACATATATGTATATATGATT | | TAAACCAATGGATTTGCCTTCAACATGGCTCTCACAA[G/C]AA | | | | | |
| TAGTTTGTGCATAATATATGCnTAC_NODE_1_length_596_ | | GTAAGTAAAGCATATATTATGCACAAACTAAATCATATATAC | | | | | |
| cov_74.407715 | | ATATATGTATTTCTTGAATACTGTACTTACAAGTTA | | | | | |
| RAD_kmer_0003535_1_TGCAGGAACAAGGGCCCATCCCAAT | 306 | TCAGGTGMTTGTTCAACCCGAACACGATCCATAATTCCCTA | G | G | A | G | GYSNP1042 |
| CGCTCATTCACGTGTAGGTTTTATGGACGCCAGCCTAAGA | | TTTTTATTCGTGTCAAAAACATCAACCCTAAGCCGCAC[A/G]G | | | | | |
| ATAATGTCAACCTGGCTACTAATTTC_NODE_length_619_ | | ACTTAGAATTGTATAACCTGAAACAATTTGGTGAGTTGTAAT | | | | | |
| cov_103.922455 | | CAAGTTGTAAATGTGTTAATGAAGTTGTGGGTTGAAA | | | | | |
| RAD_kmer_0003535_1_TGCAGGAACAAGGGCCCATCCCAAT | 404 | CCTGAAACAATTTGGTGAGTTGTAATCAAGTTGTAAATGTGT | A | A | G | A | GYSNP1043 |
| CGCTCATTCACGTGTAGGTTTTATGGACGCCAGCCTAAGA | | TAATGAAGTTGTGGGTTGAAAATGGGTTAAGCATGTTA[G/A]C | | | | | |
| ATAATGTCAACCTGGCTACTAATTTC_NODE_length_619_ | | GGGTTGACCTGTTTAACTAAACAGGTTGGTGGGTCGACCTAT | | | | | |
| cov_103.922455 | | TTTGTTAAACGGGCTATATATGTCAACCCTAACACGA | | | | | |
| RAD_kmer_0003537_1_TGCAGGACATAAAGAAAGAGTTGC | 130 | TTAAATGAGTTTCATTAATTAATAGATTTTGTAACGGTCAAAT | W | A | A | W | GYSNP1044 |
| AATTTTCAAGGGGCGTGCCAAAAAAATTATATCTGAAAAC | | TGATAAATTTCAAATGGTTATTCTTGTGATTTTCCTT[A/T]ATT | | | | | |
| TTGATTTATACTGAAAACTTAACAACT_NODE_length_416_ | | ATATGTATGCATCTTTTATGTATATTTGCAGACTCAAGTATGT | | | | | |
| cov_51.737980 | | ATGTATACATGTACCGTACTTTACACATGAACAC | | | | | |
| RAD_kmer_0003537_1_TGCAGGACATAAAGAAAGAGTTGC | 198 | GTGATTTTCCTTAATTATATGTATGCATCTTTTATGTATATTTG | Y | T | T | Y | GYSNP1045 |
| AATTTTCAAGGGGCGTGCCAAAAAAATTATATCTGAAAAC | | CAGACTCGTATGTATGTATACATGTACCGTACTT[T/C]ACA | | | | | |
| TTGATTTATACTGAAAACTTAACAACT_NODE_1_length_416_ | | CATGAACACAAACTAGACATAAGAATATGGTAATACTATAAT | | | | | |
| cov_51.737980 | | GTATGGAATCTCTTATAATAATTAGAATTTTTTGT | | | | | |
| RAD_kmer_0003550_1_TGCAGGCTCATTTGAACACATAGAT | 241 | ACAAGATCTTAAAAGTACATCCATCCTCAACTATATGAATA | G | G | R | — | GYSNP1046 |
| AATAGCGCAAAATAAAACGGGTCAAAAGTAGCCAAACAT | | TAGTCGGCAGCAGGAAGAAGAGATGGTGATGATTTACT[G/A] | | | | | |
| ATACTCTTTAAGTGTTTACAAATTCAT_NODE_2_length_340_ | | AACATTAAAGATGCTAATTGTTGGGTTCAGATTGATGTTGTTA | | | | | |
| cov_49.994116 | | TCATAAGACAACAATAAATTTAAATTATTGGGTATTC | | | | | |
| RAD_kmer_0003552_1_TGCAGGTTGTGAATGGTAGGAATGT | 511 | TTCAGATTCCTAGAAGGAAGAAAGTGCACATGCTTAGGTTTC | T | T | C | — | GYSNP1047 |
| GAAATGATATTAATTAAGTCGTTAGAATTGTGTTTTTTCTT | | AACAGCAATTTCTATAAATAAGCAATTCCAACAGGAAA[C/T] | | | | | |
| ATGCATGGCTATTTAAGTCAGCCTC_NODE_1_length_607_cov_ | | AGAGATTAATATGTCGTATACTAAGAGATGAGAGGCTGACTT | | | | | |
| 3.871498 | | AAATAGCCATGCATAAGAAAAAACACAATTCTAACGAC | | | | | |
| RAD_kmer_0003557_1_TGCAGGTCAAACGAAATCTTTAAAC | 173 | ACTTGTCTCTTCAAGCCAGTAATGATAATATTGACTTATGTTG | A | G | R | R | GYSNP1048 |
| AAAATGGGGTTTGAAGCATAAAACAGAAATCAAACCGGA | | GTATTTATCCAAGCTTCATGATCAAATATGTTCTCCC[G/A]TA | | | | | |
| GTTGGTTACCTTTCTACGCAACTAGAA_NODE_1_length_572_ | | GGACCAAGCGCGTTCGTTTGCATTGACATTTTTTTrrATATAT | | | | | |
| cov_92.022728 | | TAATGGATGAGAAATGTTCAAGTAGCTTTTGAACC | | | | | |
| RAD_kmer_0003557_1_TGCAGGTCAAACGAAATCTTTAAAC | 272 | TTTGCATTGACATTTTTTTTTATATATTAATGGATGAGAAATG | — | T | T | Y | GYSNP1049 |
| AAAATGGGGTTTGAAGCATAAAACAGAAATCAAACCGGA | | TTCAAGTAGCTTTGAACCATTTTGACATTACAAGTT[T/C]ATC | | | | | |
| GTTGGTTACCTTTCTACGCAACTAGAA_NODE_1_length_572_ | | CCCTCCTTTAGAATTTTCTTCTGCTCCATTTACTTTATCATTCA | | | | | |
| cov_92.022728 | | TATTAATGGTACATAAAGGGGAAAGATCCGTTT | | | | | |
| RAD_kmer_0003561_1_TGCAGGATCTGTACATATCTCAAAC | 383 | AGATGCTATTCTTGAACCTGAGGGCCCTAAGGTACTCTATCA | C | T | C | C | GYSNP1050 |
| GAAGAACCAATCATTTAAACATATAATCATTCTAAATAAA | | ATTTGGTATGTTTTGATCTTATTATGGTTGATTTGTAA[C/T]TA | | | | | |
| AACAGATATTTGAGAGATTTGTTGCA_NODE_2_length_441_ | | AATGAHAATGATATTATATGCAGAAAATGGAAATTGTGAAA | | | | | |
| cov_3.609977 | | CATGACTTCCAAGTTGTTTTTTTTTTTTTTTTTTTT | | | | | |
| RAD_kmer_0003564_1_TGCAGGCTACCATCAAGCCTAACCA | 258 | CATTCGCTAAAATCACACAAATCCACATTTCAATTCACATCAT | T | — | C | T | GYSNP1051 |
| GCTTATCATCCGTTCACAGAAACGTGAGGAAACCACAACT | | TAAGCACGTGATTGGTTATCGTAACCAGATCATTAAA[C/T]AC | | | | | |
| GCTGACGCAGCAATCTCTCGCCGTTT_NODE_1_length_571_ | | GTGACGGGTATTGTAAGTAGCAGAGTGCCTTAAAAGTTAAAA | | | | | |
| cov_115.099823 | | CATCTAATATATATTTTTAAACTTTGAGTTCTAGTT | | | | | |
| RAD_kmer_0003567_1_TGCAGGTAGGGGTCCTCTATACCAA | 156 | ACAAAATAAGTGTTATGTCTTTTGAACGCAGATTACAAGAGC | K | T | T | K | GYSNP1052 |
| GGACCAGGAGGTGCATTCATTGGTGGGCCGCGCCAGGCA | | GGAACTGTTGATACTTGGACAAGAGATGGTTCTCCAAA[T/G]T | | | | | |
| TCATAATGTTGAGGGGGAACATTGGAG_NODE_length_283_ | | TTGAAGATGGGGTTCGCCCTAATGCTGACAAGTGGCAGGGGG | | | | | |
| cov_217.427567 | | ACCCACAGCAATACTTCAACTCCAATGTTCCCCCTCA | | | | | |
| RAD_kmer_0003568_1_TGCAGGAAAAATTCACATTTTTCGG | 147 | TCATGAGAATACATTGGATCTTGGACAGCGCATCACCAGGTA | — | T | C | T | GYSNP1053 |
| TCACATTTACACCTTTTTAAAGCATAAACAAGAAAATAAA | | TGCTCATGTGCTTGACCAATTGGGCTATCGCATGTCTG[C/T]T | | | | | |
| AAACGGAGTCAAACAAAAATATGTTA_NODE_1_length_602_ | | AGATCAATATACGGAGATAGTAGTTTATTATACAAACCTCAA | | | | | |
| cov_107.320595 | | TAGAATCATTTTTTTATTCTCTTCTCGGGGAAGAGGG | | | | | |
| RAD_kmer_0003568_1_TGCAGGAAAAATTCACATTTTTCGG | 265 | CCTCAATAGAATCATTTTTTTATTCTCTTCTCGGGGAAGAGGG | T | T | A | T | GYSNP1054 |
| TCACATTTACACCTTTTTAAAGCATAAACAAGAAAATAAA | | GACATCTACACATAGTAATAAATAATAATCATATTTTA[A/T]AA | | | | | |
| AAACGGAGTCAAACAAAAATATGTTA_NODE_1_length_602_ | | GTTAAAAGACCTCATACTTTACTGTCTTTCTGAAAATTTTCTG | | | | | |
| cov_107.320595 | | GAGTGTTAAATTGATEATACTCTTTTAACTCGCAA | | | | | |
| RAD_kmer_0003568_1_TGCAGGAAAAATTCACATTTTTCGG | 328 | AATAATAATCATATTTAAAAGTTAAAAGACCTCATACTTTAC | — | C | T | C | GYSNP1055 |
| TCACATTTACACCTTTTTAAAGCATAAACAAGAAAATAAA | | TGTCTTTCTGAAAATTTTCTGGAGTTGTTAAATTGATTA[T/C]AC | | | | | |
| AAACGGAGTCAAACAAAAATATGTTA_NODE_1_length_602_ | | TCTTTTAACTCGCAAGATATTTGTTACAGCATAGCAGTTAAAG | | | | | |
| cov_107.320595 | | TGAGCTTATTATTCAACTGTTTAGTTGAACTAGAG | | | | | |
| RAD_kmer_0003570_1_TGCAGGTAAAGTGATGGTCTACGTT | 345 | GCAACAAAATCTTCATTATCAATATTTGTTCGGCTAATGCAG | S | G | S | G | GYSNP1056 |
| CAAGGTCCTGCTCCAGGAATCGGTTTTATGGTTACCAGAT | | GATCCAATTGGGAAACTGTTGTGTATTTGTTTGATTTT[C/G]AC | | | | | |
| TTCAAATCGTTGCTGTTGATCACCAG_NODE_3_length_425_ | | CGTTGCTCCATGCATGCTTCTCGAGTCAGAGGTTGAGAATTTC | | | | | |
| cov_35.689411 | | AGAGTAATGTCCCGCTTACGAATCTGCAAGTTAGA | | | | | |
| RAD_kmer_0003572_1_TGCAGGATTATTATTAGTTATTATTG | 126 | AAATTTTTAAGCTTCAAGTATATAGGAGTTGCAAGATACAAT | C | M | M | C | GYSNP1057 |
| TTTGTTCTTATTGAAAAAAGTGTTTTATTTGAATAAATTGT | | GTATTATTTGAATGCGTCGGTTTATTTAAAAAAAAAAAA[C/A]T | | | | | |
| AGGTTCCAGCATCCAAAATCAGCC_NODE_2_length_454_cov_ | | CAAAATTATGTTAAAATTGTGGACAAAAAGTGTTTGGGTTTG | | | | | |
| 71.579292 | | CACACAAAACTACATGTTACCCACGTGCTTTCTTTTG | | | | | |
| RAD_kmer_0003578_1_TGCAGGAAGTTCATTCAAATCGCAA | 363 | TACAGTTGGTAGCTAAAGTCAGGAATAAATGTAATGTTAGTT | M | M | A | M | GYSNP1058 |
| CCTTGCAAAGGTAGTTCTATATCTTTCAATGGCTTTGAGCT | | TCTATTTTGTGGAGCTTGCCTTTCTGATATGTGGATCT[A/C]AC | | | | | |
| CCCGCCACCCTCTGTCTGCACAGGT_NODE_length_625_cov_ | | GATTTAAGGATTCTAAAAAATCGAAGATGCAAGTGTTTTATA | | | | | |
| 126.820801 | | TATCTGTGAACTAATACCCATCTAAAATGAACCAAT | | | | | |
| RAD_kmer_0003578_1_TGCAGGAAGTTCATTCAAATCGCAA | 462 | AAAATCGAAGATGCAAGTGTTTTATATATCTGTGAACTAATA | R | R | G | R | GYSNP1059 |
| CCTTGCAAAGGTAGTTCTATATCTTTCAATGGCTTTGAGCT | | CCCATCTAAAATGAACCAATTTTTTTTCCATGATTCAT[G/A]TA | | | | | |
| CCCGCCACCCTCTGTCTGCACAGGT_NODE_1_length_625_cov_ | | TTAAATATGTAAAGTTGTTATACCTTGTCAGGAGGAAGACTT | | | | | |
| 126.820801 | | GGAGAAGAAATTGATTTTCATCCAACTACCACCAAC | | | | | |
| RAD_kmer_0003581_1_TGCAGGTATGGTCAGGTATGGAACC | 376 | AGGATTTATCTTGGCTCCAATCATTCGAGATTGGTTCATACGA | R | A | G | A | GYSNP1060 |
| GGGTACACGATACCCAGTTTCAGTTTTTTTTGGGTCCGATC | | ATTGAAAAGTTTGATAAGAATAAGAATTAGGCCGCTC[G/A]A | | | | | |
| ATACCCCGTTTTTTGTGCACCTCTA_NODE_1_length_575_cov_ | | GCTTTTATGTTTCTAGCTTGACTTGAATGATTCGAGTCTGAGC | | | | | |
| 77.396523 | | TTGAGCCCAAATAACTTTATTATATCTTGACTCATT | | | | | |
| RAD_kmer_0003583_1_TGCAGGCTAGTCATCGTGTAGAATC | 120 | GTTAGTGGAGTATATTTTTTTAACGATATCCACAATGACCATA | R | R | R | G | GYSNP1061 |
| TGATGAAAGAAGACCTCTACTTCCTACTTGAATTGACCAG | | ATACGCCGTAAAGGGTTTTCTTTTCTTCTTAAAATGA[A/G]AT | | | | | |
| GGTAAGTTTCTATAATTACTATGTTG_NODE_2_length_398_ | | TTTTCTGTACCCTCGCCAACTAGAGCCAAAAGAATTCTTTGAA | | | | | |
| cov_54.894474 | | GAAGAAACCTGCATTGACCTTCATATATAAACAGG | | | | | |
| RAD_kmer_0003586_1_TGCAGGCTGCAGGCTTACCATTTAC | 304 | TAGAATATATAGTTGTCGTACATAGCTGTTGCATCCTATGTAT | A | A | T | A | GYSNP1062 |
| TATTCTAATTTCATTTTATAAATGACATGTTTCACAGTTTA | | ACGGCCTTTTGGAGAATCTTATGAAATTGTCATTTGA[T/A]AA | | | | | |
| TTGGATTTCTTGAACCGTATATGGA_NODE_length_632_cov_ | | AAACATCAAAACAGTTGCTAACTGATCCTCACACAAGACATT | | | | | |
| 129.993668 | | GACTAGTCAAATGAAACATTTTTCTTGTTTTGGAGA | | | | | |
| RAD_kmer_0003589_1_TGCAGGTCCGGAGAGGATTGCTAAG | 284 | GAGGACCGACTCCTGTGAATAGTCCCTTTATTCCTCCATCCCT | W | W | A | T | GYSNP1063 |
| CATGTCTGGTAATGAAAGTAGCCTTGCTAAAATTCTTGGT | | GTTCCATGATCAATGATCATAACAATGCATCTTGTCA[A T]AT | | | | | |
| GCAAACTTTGCAGCGGGGTTTGTTGC_NODE_1_length_612_ | | TTGTAATCAAATAATTGAAAAAAAAACTGATGTCGATAAAGT | | | | | |
| cov_74.854576 | | GGACAAATGATAAATACCGCCATATCTCAACAAGTG | | | | | |
| RAD_kmer_0003590_1_TGCAGGTACTATTACCGGAGTTGCT | 95 | GTATGTATGTATATGGCGTGCCAACAATAACCAGCCTACACT | — | A | C | A | GYSNP1064 |
| AACGCGGAAGCTGTCAAGTTGTCAGATTAATGTCATGTCC | | CTAATTTGGTAATTTCTACCTTTGATCCACCACACCAG[C/A]A | | | | | |
| ACTTTCCTTTTTTAACGATGCTTTAA_NODE_1_length_385_cov_ | | ACATGACCATCTAATAACTGGCAAAAAGATTAAGCCTACTGG | | | | | |
| 109.132469 | | AATTATAATGAACACCAAGAATTCATCGTTTGCTGAA | | | | | |
| RAD_kmer_0003590_1_TGCAGGTACTATTACCGGAGTTGCT | 194 | CTGGCAAAAAGATTAAGCCTACTGGAATTATAATGAACACCA | — | T | A | T | GYSNP1065 |
| AACGCGGAAGCTGTCAAGTTGTCAGATTAATGTCATGTCC | | AGAATTCATCGTTTGCTGAAACTAAAAArACCGAATAC[AT]T | | | | | |
| ACTTTCCTTTTTTAACGATGCTTTAA_NODE_length_385_cov_ | | CCAACAAAGTCGCATTTTTTGAAGCTTAGTTAACACTCAACA | | | | | |
| 109.132469 | | GCATCAAGCATAACAAAAGGCTCCAACAAAGTAACAA | | | | | |

TABLE 10
Information Regarding Guayule SNP Molecular Markers

| Marker | Major.Allele.Frquency | GenotypeNo | No. of obs. | GeneDiversity | Heterozygosity | PIC |
|---|---|---|---|---|---|---|
| SNP001 | 0.500 | 1 | 12 | 0.500 | 1.000 | 0.375 |
| SNP002 | 0.500 | 1 | 12 | 0.500 | 1.000 | 0.375 |
| SNP003 | 1.000 | 1 | 12 | 0.000 | 0.000 | 0.000 |
| SNP004 | 1.000 | 1 | 12 | 0.000 | 0.000 | 0.000 |
| SNP005 | 1.000 | 1 | 12 | 0.000 | 0.000 | 0.000 |
| SNP006 | 0.542 | 3 | 12 | 0.497 | 0.417 | 0.373 |
| SNP007 | 0.500 | 1 | 12 | 0.500 | 1.000 | 0.375 |
| SNP008 | 1.000 | 1 | 11 | 0.000 | 0.000 | 0.000 |
| SNP009 | 0.958 | 2 | 12 | 0.080 | 0.083 | 0.077 |
| SNP010 | 1.000 | 1 | 12 | 0.000 | 0.000 | 0.000 |
| SNP011 | 0.792 | 3 | 12 | 0.330 | 0.250 | 0.275 |
| SNP012 | 0.958 | 2 | 12 | 0.080 | 0.083 | 0.077 |
| SNP013 | 0.792 | 2 | 12 | 0.330 | 0.417 | 0.275 |
| SNP014 | 0.958 | 2 | 12 | 0.080 | 0.083 | 0.077 |
| SNP015 | 0.500 | 1 | 12 | 0.500 | 1.000 | 0.375 |
| SNP016 | 0.792 | 3 | 12 | 0.330 | 0.083 | 0.275 |
| SNP017 | 1.000 | 1 | 12 | 0.000 | 0.000 | 0.000 |
| SNP018 | 0.583 | 2 | 12 | 0.486 | 0.833 | 0.368 |
| SNP019 | 0.500 | 1 | 12 | 0.500 | 1.000 | 0.375 |
| SNP020 | 0.500 | 1 | 12 | 0.500 | 1.000 | 0.375 |
| SNP021 | 0.500 | 1 | 12 | 0.500 | 1.000 | 0.375 |
| SNP022 | 0.500 | 1 | 12 | 0.500 | 1.000 | 0.375 |
| SNP023 | 1.000 | 1 | 12 | 0.009 | 0.000 | 0.000 |
| SNP024 | 0.917 | 2 | 12 | 0.153 | 0.167 | 0.141 |
| SNP025 | 1.000 | 1 | 12 | 0.000 | 0.000 | 0.000 |
| SNP026 | 0.500 | 1 | 12 | 0.500 | 1.000 | 0.375 |
| SNP027 | 0.500 | 1 | 12 | 0.500 | 1.000 | 0.375 |
| SNP028 | 0.500 | 1 | 12 | 0.500 | 0.000 | 0.375 |
| SNP029 | 1.000 | 1 | 12 | 0.000 | 0.000 | 0.000 |
| SNP030 | 1.000 | 1 | 12 | 0.000 | 0.000 | 0.000 |
| SNP031 | 1.000 | 1 | 12 | 0.000 | 0.000 | 0.000 |
| SNP032 | 1.000 | 1 | 10 | 0.000 | 0.000 | 0.000 |
| SNP033 | 1.000 | 1 | 8 | 0.000 | 0.000 | 0.000 |
| SNP034 | 0.958 | 2 | 12 | 0.080 | 0.083 | 0.077 |
| SNP035 | 1.000 | 1 | 8 | 0.000 | 0.000 | 0.000 |
| SNP036 | 1.000 | 1 | 12 | 0.000 | 0.000 | 0.000 |
| SNP037 | 1.000 | 1 | 12 | 0.000 | 0.000 | 0.000 |
| SNP038 | 0.542 | 2 | 12 | 0.497 | 0.917 | 0.373 |
| SNP039 | 0.500 | 1 | 12 | 0.500 | 1.000 | 0.375 |
| SNP040 | 0.917 | 2 | 12 | 0.153 | 0.000 | 0.141 |
| SNP041 | 0.500 | 1 | 12 | 0.500 | 1.000 | 0.375 |
| SNP042 | 0.500 | 1 | 12 | 0.500 | 1.000 | 0.375 |
| SNP043 | 0.833 | 2 | 12 | 0.278 | 0.333 | 0.239 |
| SNP044 | 0.500 | 1 | 12 | 0.500 | 1.000 | 0.375 |
| SNP045 | 0.833 | 2 | 12 | 0.278 | 0.000 | 0.239 |
| SNP046 | 1.000 | 1 | 12 | 0.000 | 0.000 | 0.000 |
| SNP047 | 0.500 | 1 | 12 | 0.500 | 1.000 | 0.375 |
| SNP048 | 0.500 | 1 | 12 | 0.500 | 1.000 | 0.375 |
| SNP049 | 0.500 | 1 | 12 | 0.500 | 0.000 | 0.375 |
| SNP050 | 1.000 | 1 | 12 | 0.000 | 0.000 | 0.000 |
| SNP051 | 1.000 | 1 | 12 | 0.000 | 0.000 | 0.000 |
| SNP052 | 1.000 | 1 | 11 | 0.000 | 0.000 | 0.000 |
| SNP053 | 1.000 | 1 | 12 | 0.000 | 0.000 | 0.000 |
| SNP054 | 0.625 | 3 | 12 | 0.469 | 0.250 | 0.359 |
| SNP055 | 0.708 | 3 | 12 | 0.413 | 0.250 | 0.328 |
| SNP056 | 0.667 | 3 | 12 | 0.444 | 0.500 | 0.346 |
| SNP057 | 0.500 | 1 | 11 | 0.500 | 1.000 | 0.375 |
| SNP058 | 0.500 | 1 | 11 | 0.500 | 1.000 | 0.375 |
| SNP059 | 0.542 | 2 | 12 | 0.497 | 0.917 | 0.373 |
| SNP060 | 0.500 | 1 | 12 | 0.500 | 1.000 | 0.375 |
| SNP061 | 0.500 | 3 | 11 | 0.500 | 0.818 | 0.375 |
| SNP062 | 0.917 | 2 | 12 | 0.153 | 0.167 | 0.141 |
| SNP063 | 0.542 | 2 | 12 | 0.497 | 0.917 | 0.373 |
| SNP064 | 0.500 | 1 | 12 | 0.500 | 1.000 | 0.375 |
| SNP065 | 0.500 | 1 | 12 | 0.500 | 1.000 | 0.375 |
| SNP066 | 0.500 | 1 | 11 | 0.500 | 0.500 | 0.375 |
| SNP067 | 0.500 | 1 | 12 | 0.500 | 1.000 | 0.375 |
| SNP068 | 1.000 | 1 | 12 | 0.500 | 0.000 | 0.000 |
| SNP069 | 0.500 | 1 | 12 | 0.500 | 1.000 | 0.375 |
| SNP070 | 0.500 | 1 | 12 | 0.500 | 1.000 | 0.375 |
| SNP071 | 0.500 | 1 | 12 | 0.500 | 1.000 | 0.375 |
| SNP072 | 1.000 | 1 | 12 | 0.000 | 0.000 | 0.000 |
| SNP073 | 0.500 | 1 | 12 | 0.500 | 1.000 | 0.375 |
| SNP074 | 0.500 | 1 | 12 | 0.500 | 1.000 | 0.375 |
| SNP075 | 0.500 | 1 | 12 | 0.500 | 1.000 | 0.375 |
| SNP076 | 0.500 | 1 | 12 | 0.500 | 1.000 | 0.375 |
| SNP077 | 0.909 | 2 | 11 | 0.165 | 0.000 | 0.152 |
| SNP078 | 1.000 | 1 | 12 | 0.000 | 0.000 | 0.000 |
| SNP079 | 0.583 | 3 | 12 | 0.486 | 0.667 | 0.368 |
| SNP080 | 0.500 | 1 | 12 | 0.500 | 1.000 | 0.375 |
| SNP081 | 0.500 | 1 | 12 | 0.500 | 1.000 | 0.375 |
| SNP082 | 0.500 | 1 | 10 | 0.500 | 1.000 | 0.375 |
| SNP083 | 0.500 | 1 | 11 | 0.500 | 1.000 | 0.375 |
| SNP084 | 1.000 | 1 | 11 | 0.000 | 0.000 | 0.000 |
| SNP085 | 0.545 | 2 | 11 | 0.496 | 0.909 | 0.373 |
| SNP086 | 0.750 | 2 | 12 | 0.375 | 0.500 | 0.305 |
| SNP087 | 1.000 | 1 | 11 | 0.000 | 0.000 | 0.000 |
| SNP088 | 0.500 | 1 | 12 | 0.500 | 1.000 | 0.375 |
| SNP089 | 0.500 | 1 | 12 | 0.500 | 1.000 | 0.375 |
| SNP090 | 0.917 | 2 | 12 | 0.153 | 0.167 | 0.141 |
| SNP091 | 0.500 | 1 | 11 | 0.500 | 1.000 | 0.375 |
| SNP092 | 0.958 | 2 | 12 | 0.080 | 0.083 | 0.077 |
| SNP093 | 0.500 | 3 | 12 | 0.500 | 0.833 | 0.375 |
| SNP094 | 1.000 | 1 | 11 | 0.000 | 0.000 | 0.000 |
| SNP095 | 0.500 | 1 | 12 | 0.500 | 1.000 | 0.375 |
| SNP096 | 0.625 | 2 | 12 | 0.469 | 0.750 | 0.359 |
| SNP097 | 0.542 | 2 | 12 | 0.497 | 0.917 | 0.373 |
| SNP098 | 0.792 | 2 | 12 | 0.330 | 0.417 | 0.275 |
| SNP099 | 0.500 | 1 | 12 | 0.500 | 1.000 | 0.375 |
| SNP100 | 0.500 | 1 | 12 | 0.500 | 1.000 | 0.375 |
| SNP101 | 0.500 | 1 | 12 | 0.500 | 1.000 | 0.375 |
| SNP102 | 0.625 | 2 | 12 | 0.469 | 0.750 | 0.359 |
| SNP103 | 0.500 | 1 | 12 | 0.500 | 1.000 | 0.375 |
| SNP104 | 0.500 | 1 | 12 | 0.500 | 1.000 | 0.375 |
| SNP105 | 0.500 | 1 | 12 | 0.500 | 1.000 | 0.375 |
| SNP106 | 0.500 | 1 | 12 | 0.500 | 1.000 | 0.375 |
| SNP107 | 0.500 | 1 | 12 | 0.500 | 1.000 | 0.375 |
| SNP108 | 0.958 | 2 | 12 | 0.080 | 0.083 | 0.077 |
| SNP109 | 1.000 | 1 | 12 | 0.000 | 0.000 | 0.000 |
| SNP110 | 0.500 | 1 | 12 | 0.500 | 1.000 | 0.375 |
| SNP111 | 0.542 | 2 | 12 | 0.497 | 0.917 | 0.373 |
| SNP112 | 0.500 | 1 | 12 | 0.500 | 1.000 | 0.375 |
| SNP113 | 0.500 | 1 | 12 | 0.500 | 1.000 | 0.375 |
| SNP114 | 0.500 | 1 | 12 | 0.500 | 1.000 | 0.375 |
| SNP115 | 1.000 | 1 | 12 | 0.000 | 0.000 | 0.000 |
| SNP116 | 0.500 | 1 | 12 | 0.500 | 1.000 | 0.375 |
| SNP117 | 0.958 | 2 | 12 | 0.080 | 0.083 | 0.077 |
| SNP118 | 1.000 | 1 | 12 | 0.000 | 0.000 | 0.000 |
| SNP119 | 0.500 | 1 | 12 | 0.500 | 1.000 | 0.375 |
| SNP120 | 0.500 | 1 | 12 | 0.500 | 1.000 | 0.375 |
| SNP121 | 0.958 | 2 | 12 | 0.083 | 0.077 | 0.080 |
| SNP122 | 0.500 | 1 | 12 | 0.500 | 1.000 | 0.375 |
| SNP123 | 0.500 | 1 | 12 | 0.500 | 1.000 | 0.375 |
| SNP124 | 0.875 | 2 | 12 | 0.219 | 0.250 | 0.195 |
| SNP125 | 0.500 | 1 | 12 | 0.500 | 1.000 | 0.375 |
| SNP126 | 1.000 | 1 | 12 | 0.000 | 0.000 | 0.000 |
| SNP127 | 0.500 | 1 | 12 | 0.500 | 1.000 | 0.375 |
| SNP128 | 0.500 | 1 | 12 | 0.500 | 1.000 | 0.375 |
| SNP129 | 0.500 | 1 | 12 | 0.500 | 1.000 | 0.375 |
| SNP130 | 0.909 | 2 | 11 | 0.165 | 0.182 | 0.152 |
| SNP131 | 0.500 | 1 | 12 | 0.500 | 1.000 | 0.375 |
| SNP132 | 0.500 | 1 | 12 | 0.500 | 1.000 | 0.375 |
| SNP133 | 0.750 | 2 | 12 | 0.375 | 0.500 | 0.305 |
| SNP134 | 0.500 | 1 | 12 | 0.500 | 1.000 | 0.375 |
| SNP135 | 0.500 | 1 | 11 | 0.500 | 1.000 | 0.375 |
| SNP136 | 0.500 | 1 | 12 | 0.500 | 1.000 | 0.375 |
| SNP137 | 0.500 | 1 | 12 | 0.500 | 1.000 | 0.375 |
| SNP138 | 0.500 | 1 | 11 | 0.500 | 1.000 | 0.375 |
| SNP139 | 0.500 | 1 | 12 | 0.500 | 1.000 | 0.375 |
| SNP140 | 0.864 | 2 | 11 | 0.236 | 0.273 | 0.208 |
| SNP141 | 0.500 | 1 | 12 | 0.500 | 1.000 | 0.375 |
| SNP142 | 0.500 | 1 | 12 | 0.500 | 1.000 | 0.375 |
| SNP143 | 0.500 | 1 | 12 | 0.500 | 1.000 | 0.375 |
| SNP144 | 0.500 | 1 | 12 | 0.500 | 1.000 | 0.375 |
| SNP145 | 0.500 | 1 | 12 | 0.500 | 1.000 | 0.375 |
| SNP146 | 0.500 | 1 | 12 | 0.500 | 1.000 | 0.375 |
| SNP147 | 0.500 | 1 | 12 | 0.500 | 1.000 | 0.375 |
| SNP148 | 0.500 | 1 | 12 | 0.500 | 1.000 | 0.375 |
| SNP149 | 0.500 | 1 | 12 | 0.500 | 1.000 | 0.375 |
| SNP150 | 0.950 | 2 | 10 | 0.095 | 0.100 | 0.090 |
| SNP151 | 0.958 | 1 | 12 | 0.080 | 0.083 | 0.077 |
| SNP152 | 0.500 | 1 | 12 | 0.500 | 1.000 | 0.375 |
| SNP153 | 0.500 | 1 | 11 | 0.500 | 1.000 | 0.375 |
| SNP154 | 0.500 | 1 | 12 | 0.500 | 1.000 | 0.375 |
| SNP156 | 0.500 | 1 | 12 | 0.500 | 1.000 | 0.375 |
| SNP157 | 0.500 | 1 | 12 | 0.500 | 1.000 | 0.375 |
| SNP158 | 0.500 | 1 | 12 | 0.500 | 1.000 | 0.375 |
| SNP159 | 0.500 | 1 | 12 | 0.500 | 1.000 | 0.375 |
| SNP160 | 0.917 | 2 | 12 | 0.153 | 0.167 | 0.141 |
| SNP161 | 0.500 | 1 | 10 | 0.500 | 1.000 | 0.375 |
| SNP162 | 0.917 | 2 | 12 | 0.153 | 0.167 | 0.141 |
| SNP163 | 1.000 | 1 | 11 | 0.000 | 0.000 | 0.000 |
| SNP164 | 0.958 | 2 | 12 | 0.080 | 0.083 | 0.077 |
| SNP165 | 0.500 | 1 | 12 | 0.500 | 1.000 | 0.375 |
| SNP166 | 0.682 | 2 | 11 | 0.434 | 0.636 | 0.340 |
| SNP167 | 0.500 | 1 | 12 | 0.500 | 1.000 | 0.375 |
| SNP168 | 0.833 | 2 | 12 | 0.278 | 0.333 | 0.239 |
| SNP169 | 0.542 | 2 | 12 | 0.497 | 0.917 | 0.373 |
| SNP170 | 0.500 | 1 | 12 | 0.500 | 1.000 | 0.375 |
| SNP171 | 0.500 | 1 | 11 | 0.500 | 1.000 | 0.375 |
| SNP172 | 0.500 | 1 | 12 | 0.500 | 1.000 | 0.375 |
| SNP173 | 0.500 | 1 | 12 | 0.500 | 1.000 | 0.375 |
| SNP174 | 0.583 | 2 | 12 | 0.486 | 0.833 | 0.368 |
| SNP175 | 0.500 | 1 | 12 | 0.500 | 1.000 | 0.375 |
| SNP176 | 0.500 | 1 | 12 | 0.500 | 1.000 | 0.375 |
| SNP177 | 0.818 | 2 | 11 | 0.298 | 0.364 | 0.253 |
| SNP178 | 0.667 | 3 | 9 | 0.444 | 0.444 | 0.346 |
| SNP179 | 0.792 | 2 | 12 | 0.330 | 0.417 | 0.275 |
| SNP180 | 0.500 | 1 | 10 | 0.500 | 1.000 | 0.375 |
| SNP181 | 0.500 | 1 | 12 | 0.500 | 1.000 | 0.375 |
| SNP182 | 0.500 | 1 | 12 | 0.500 | 1.000 | 0.375 |
| SNP183 | 0.500 | 1 | 12 | 0.500 | 1.000 | 0.375 |
| SNP184 | 0.500 | 1 | 12 | 0.500 | 1.000 | 0.375 |
| SNP185 | 0.500 | 1 | 12 | 0.500 | 1.000 | 0.375 |
| SNP186 | 1.000 | 1 | 12 | 0.000 | 0.000 | 0.000 |
| SNP187 | 1.000 | 1 | 9 | 0.000 | 0.000 | 0.000 |
| SNP188 | 0.500 | 1 | 11 | 0.500 | 1.000 | 0.375 |
| SNP189 | 0.500 | 1 | 12 | 0.500 | 1.000 | 0.375 |
| SNP190 | 0.500 | 1 | 12 | 0.500 | 1.000 | 0.375 |
| SNP191 | 0.500 | 1 | 12 | 0.500 | 1.000 | 0.375 |
| SNP192 | 0.500 | 1 | 12 | 0.500 | 1.000 | 0.375 |
| SNP193 | 0.500 | 1 | 12 | 0.500 | 1.000 | 0.375 |
| SNP194 | 0.500 | 1 | 12 | 0.500 | 1.000 | 0.375 |
| SNP195 | 0.500 | 1 | 12 | 0.500 | 1.000 | 0.375 |
| SNP196 | 0.500 | 1 | 12 | 0.500 | 1.000 | 0.375 |
| SNP197 | 0.583 | 3 | 12 | 0.486 | 0.667 | 0.368 |
| SNP198 | 0.500 | 1 | 12 | 0.500 | 1.000 | 0.375 |
| SNP199 | 0.500 | 1 | 12 | 0.500 | 1.000 | 0.375 |
| SNP200 | 0.500 | 1 | 12 | 0.500 | 1.000 | 0.375 |
| SNP201 | 0.500 | 1 | 12 | 0.500 | 1.000 | 0.375 |
| SNP202 | 0.591 | 2 | 11 | 0.483 | 0.818 | 0.367 |
| SNP203 | 0.500 | 1 | 12 | 0.500 | 1.000 | 0.375 |
| SNP204 | 0.500 | 1 | 9 | 0.500 | 1.000 | 0.375 |
| SNP205 | 0.500 | 1 | 12 | 0.500 | 1.000 | 0.375 |
| SNP206 | 0.500 | 1 | 12 | 0.500 | 1.000 | 0.375 |
| SNP207 | 0.545 | 2 | 11 | 0.496 | 0.909 | 0.373 |
| SNP208 | 0.625 | 2 | 12 | 0.469 | 0.750 | 0.359 |
| SNP209 | 0.500 | 1 | 12 | 0.500 | 1.000 | 0.375 |
| SNP210 | 0.875 | 2 | 12 | 0.219 | 0.250 | 0.195 |
| SNP211 | 1.000 | 1 | 11 | 0.000 | 0.000 | 0.000 |
| SNP212 | 1.000 | 1 | 8 | 0.000 | 0.000 | 0.000 |
| SNP213 | 0.500 | 1 | 12 | 0.500 | 1.000 | 0.375 |
| SNP214 | 0.500 | 1 | 12 | 0.500 | 1.000 | 0.375 |
| SNP215 | 0.500 | 1 | 11 | 0.500 | 1.000 | 0.375 |
| SNP216 | 0.500 | 1 | 11 | 0.500 | 1.000 | 0.375 |
| SNP217 | 0.500 | 1 | 12 | 0.500 | 1..000 | 0.375 |
| SNP218 | 0.500 | 1 | 12 | 0.500 | 1.000 | 0.375 |
| SNP219 | 0.500 | 1 | 12 | 0.500 | 1.000 | 0.375 |
| SNP221 | 0.500 | 1 | 11 | 0.500 | 1.000 | 0.375 |
| SNP222 | 0.500 | 1 | 12 | 0.500 | 1.000 | 0.375 |
| SNP223 | 0.833 | 2 | 12 | 0.278 | 0.333 | 0.239 |
| SNP224 | 0.500 | 1 | 12 | 0.500 | 1.000 | 0.375 |
| SNP225 | 1.000 | 1 | 12 | 0.000 | 0.000 | 0.000 |
| SNP226 | 0.500 | 1 | 12 | 0.500 | 1.000 | 0.375 |
| SNP227 | 0.500 | 1 | 12 | 0.500 | 1.000 | 0.375 |
| SNP228 | 1.000 | 1 | 9 | 0.000 | 0.000 | 0.000 |
| SNP229 | 0.500 | 1 | 12 | 0.500 | 1.000 | 0.375 |
| SNP230 | 0.500 | 1 | 11 | 0.500 | 1.000 | 0.375 |
| SNP231 | 0.500 | 1 | 12 | 0.500 | 1.000 | 0.375 |
| SNP232 | 0.950 | 2 | 10 | 0.095 | 0.100 | 0.090 |
| SNP233 | 0.500 | 1 | 11 | 0.500 | 1.000 | 0.375 |
| SNP234 | 0.917 | 2 | 12 | 0.153 | 0.167 | 0.141 |
| SNP235 | 0.917 | 2 | 12 | 0.153 | 0.167 | 0.141 |
| SNP236 | 0.500 | 1 | 12 | 0.500 | 1.000 | 0.375 |
| SNP237 | 0.500 | 1 | 12 | 0.500 | 1.000 | 0.375 |
| SNP238 | 0.500 | 1 | 12 | 0.500 | 1.000 | 0.375 |
| SNP239 | 0.500 | 1 | 12 | 0.500 | 1.000 | 0.375 |
| SNP240 | 0.813 | 2 | 8 | 0.305 | 0.375 | 0.258 |
| SNP241 | 0.500 | 1 | 8 | 0.500 | 1.000 | 0.375 |
| SNP242 | 0.500 | 1 | 12 | 0.500 | 1.000 | 0.375 |
| SNP243 | 0.833 | 2 | 12 | 0.278 | 0.333 | 0.239 |
| SNP244 | 0.500 | 1 | 11 | 0.500 | 1.000 | 0.375 |
| SNP245 | 0.500 | 1 | 12 | 0.500 | 1.000 | 0.375 |
| SNP246 | 0.500 | 1 | 11 | 0.500 | 1.000 | 0.375 |
| SNP247 | 0.500 | 1 | 10 | 0.500 | 1.000 | 0.375 |
| SNP248 | 0.500 | 1 | 11 | 0.500 | 1.000 | 0.375 |
| SNP249 | 0.500 | 1 | 12 | 0.500 | 1.000 | 0.375 |
| SNP250 | 0.556 | 3 | 9 | 0.494 | 0.444 | 0.372 |
| SNP251 | 0.500 | 1 | 11 | 0.500 | 1.000 | 0.375 |
| SNP252 | 0.500 | 1 | 12 | 0.500 | 1.000 | 0.375 |
| SNP253 | 0.600 | 2 | 10 | 0.480 | 0.800 | 0.365 |
| SNP254 | 0.958 | 2 | 12 | 0.080 | 0.083 | 0.077 |
| SNP255 | 0.500 | 1 | 12 | 0.500 | 1.000 | 0.375 |
| SNP256 | 0.500 | 1 | 12 | 0.500 | 1.000 | 0.375 |
| SNP257 | 0.500 | 1 | 10 | 0.500 | 1.000 | 0.375 |
| SNP258 | 0.500 | 1 | 11 | 0.500 | 1.000 | 0.375 |
| SNP259 | 0.500 | 1 | 11 | 0.500 | 1.000 | 0.375 |
| SNP260 | 0.500 | 1 | 11 | 0.500 | 1.000 | 0.375 |
| SNP262 | 0.500 | 1 | 12 | 0.500 | 1.000 | 0.375 |
| SNP263 | 1.000 | 1 | 12 | 0.000 | 0.000 | 0.000 |
| SNP264 | 0.500 | 1 | 12 | 0.500 | 1.000 | 0.375 |
| SNP265 | 0.750 | 2 | 12 | 0.375 | 0.500 | 0.305 |
| SNP266 | 0.500 | 1 | 9 | 0.500 | 1.000 | 0.375 |
| SNP267 | 0.708 | 2 | 12 | 0.413 | 0.583 | 0.328 |
| SNP268 | 0.909 | 2 | 11 | 0.165 | 0.000 | 0.152 |
| SNP269 | 0.583 | 2 | 12 | 0.486 | 0.833 | 0.368 |
| SNP270 | 0.500 | 1 | 12 | 0.500 | 1.000 | 0.375 |
| SNP271 | 0.875 | 2 | 12 | 0.219 | 0.250 | 0.195 |
| SNP272 | 0.500 | 1 | 11 | 0.500 | 1.000 | 0.375 |
| SNP273 | 0.500 | 1 | 12 | 0.500 | 1.000 | 0.375 |
| SNP274 | 1.000 | 1 | 12 | 0.000 | 0.000 | 0.000 |
| SNP275 | 0.500 | 1 | 12 | 0.500 | 1.000 | 0.375 |
| SNP276 | 0.500 | 1 | 12 | 0.500 | 1.000 | 0.375 |
| SNP277 | 1.000 | 1 | 12 | 0.000 | 0.000 | 0.000 |
| SNP278 | 1.000 | 1 | 12 | 0.000 | 0.000 | 0.000 |
| SNP279 | 0.500 | 1 | 12 | 0.500 | 1.000 | 0.375 |
| SNP280 | 0.889 | 2 | 9 | 0.198 | 0.000 | 0.178 |
| SNP281 | 0.500 | 1 | 12 | 0.500 | 1.000 | 0.375 |
| SNP282 | 1.000 | 1 | 10 | 0.000 | 0.000 | 0.000 |
| SNP284 | 0.500 | 1 | 11 | 0.500 | 1.000 | 0.375 |
| SNP285 | 0.958 | 2 | 12 | 0.080 | 0.083 | 0.077 |
| SNP286 | 0.500 | 1 | 10 | 0.500 | 1.000 | 0.375 |
| SNP287 | 0.750 | 2 | 12 | 0.375 | 0.500 | 0.305 |
| SNP288 | 0.500 | 1 | 11 | 0.500 | 1.000 | 0.375 |
| SNP289 | 0.542 | 2 | 12 | 0.497 | 0.917 | 0.373 |
| SNP290 | 0.500 | 1 | 12 | 0.500 | 1.000 | 0.375 |
| SNP291 | 1.000 | 1 | 12 | 0.000 | 0.000 | 0.000 |
| SNP292 | 1.000 | 1 | 12 | 0.000 | 0.000 | 0.000 |
| SNP293 | 0.500 | 1 | 11 | 0.500 | 1.000 | 0.375 |
| SNP294 | 0.500 | 1 | 9 | 0.500 | 1.000 | 0.375 |
| SNP295 | 1.000 | 1 | 9 | 0.000 | 0.000 | 0.000 |
| SNP296 | 0.500 | 1 | 12 | 0.500 | 1.000 | 0.375 |
| SNP297 | 0.500 | 1 | 12 | 0.500 | 1.000 | 0.375 |
| SNP298 | 0.625 | 2 | 12 | 0.469 | 0.750 | 0.359 |
| SNP299 | 0.958 | 2 | 12 | 0.080 | 0.083 | 0.077 |
| SNP300 | 0.727 | 2 | 11 | 0.397 | 0.545 | 0.318 |
| SNP301 | 0.958 | 2 | 12 | 0.080 | 0.083 | 0.077 |
| SNP302 | 0.625 | 2 | 12 | 0.469 | 0.750 | 0.359 |
| SNP303 | 0.500 | 1 | 12 | 0.500 | 1.000 | 0.375 |
| SNP304 | 0.917 | 1 | 12 | 0.153 | 0.167 | 0.141 |
| SNP305 | 0.500 | 1 | 10 | 0.500 | 1.000 | 0.375 |
| SNP306 | 1.000 | 1 | 12 | 0.000 | 0.000 | 0.000 |
| SNP307 | 0.500 | 1 | 12 | 0.500 | 1.000 | 0.375 |
| SNP308 | 1.060 | 1 | 12 | 0.000 | 0.000 | 0.000 |
| SNP309 | 0.500 | 1 | 12 | 0.500 | 1.000 | 0.375 |
| SNP310 | 0.500 | 1 | 12 | 0.500 | 1.000 | 0.375 |
| SNP312 | 0.600 | 1 | 10 | 0.480 | 0.800 | 0.365 |
| SNP313 | 0.500 | 1 | 12 | 0.500 | 1.000 | 0.375 |
| SNP314 | 0.625 | 2 | 12 | 0.469 | 0.750 | 0.359 |
| SNP315 | 1.000 | 1 | 12 | 0.000 | 0.000 | 0.000 |
| SNP316 | 0.500 | 1 | 10 | 0.500 | 1.000 | 0.375 |
| SNP317 | 1.000 | 1 | 11 | 0.000 | 0.000 | 0.000 |
| SNP318 | 1.000 | 1 | 12 | 0.000 | 0.000 | 0.000 |
| SNP319 | 0.792 | 2 | 12 | 0.330 | 0.417 | 0.275 |
| SNP320 | 0.607 | 3 | 12 | 0.444 | 0.500 | 0.346 |
| SNP321 | 0.917 | 1 | 12 | 0.153 | 0.167 | 0.141 |
| SNP322 | 0.500 | 1 | 12 | 0.500 | 1.000 | 0.375 |
| SNP323 | 0.500 | 1 | 12 | 0.500 | 1.000 | 0.375 |
| SNP324 | 1.000 | 1 | 12 | 0.000 | 0.000 | 0.000 |
| SNP325 | 1.000 | 1 | 12 | 0.000 | 0.000 | 0.000 |
| SNP326 | 0.500 | 1 | 11 | 0.500 | 1.000 | 0.375 |
| SNP327 | 0.864 | 2 | 11 | 0.236 | 0.273 | 0.208 |
| SNP328 | 0.500 | 1 | 11 | 0.500 | 1.000 | 0.375 |
| SNP329 | 0.545 | 2 | 11 | 0.496 | 0.909 | 0.373 |
| SNP330 | 0.583 | 2 | 12 | 0.486 | 0.000 | 0.368 |
| SNP331 | 0.500 | 1 | 9 | 0.500 | 1.000 | 0.375 |
| SNP332 | 0.500 | 1 | 11 | 0.500 | 1.000 | 0.375 |
| SNP333 | 0.958 | 2 | 12 | 0.080 | 0.083 | 0.077 |
| SNP334 | 0.500 | 1 | 12 | 0.500 | 1.000 | 0.375 |
| SNP335 | 0.500 | 1 | 12 | 0.500 | 1.000 | 0.375 |
| SNP336 | 0.500 | 1 | 12 | 0.500 | 1.000 | 0.375 |
| SNP337 | 0.500 | 1 | 12 | 0.500 | 1.000 | 0.375 |
| SNP338 | 0.545 | 2 | 11 | 0.496 | 0.909 | 0.373 |
| SNP339 | 0.792 | 2 | 12 | 0.330 | 0.417 | 0.275 |
| SNP340 | 0.917 | 2 | 12 | 0.153 | 0.000 | 0.141 |
| SNP341 | 1.000 | 1 | 12 | 0.000 | 0.000 | 0.000 |
| SNP342 | 0.563 | 2 | 8 | 0.492 | 0.875 | 0.371 |
| SNP343 | 1.000 | 1 | 12 | 0.000 | 0.000 | 0.000 |
| SNP344 | 0.650 | 2 | 10 | 0.455 | 0.700 | 0.351 |
| SNP345 | 0.500 | 1 | 12 | 0.500 | 1.000 | 0.375 |
| SNP346 | 1.000 | 1 | 11 | 0.000 | 0.000 | 0.000 |
| SNP347 | 0.571 | 2 | 7 | 0.490 | 0.857 | 0.370 |
| SNP348 | 1.000 | 1 | 11 | 0.000 | 0.000 | 0.000 |
| SNP349 | 0.545 | 2 | 11 | 0.496 | 0.909 | 0.373 |
| SNP350 | 0.500 | 1 | 11 | 0.500 | 1.000 | 0.375 |
| SNP351 | 1.000 | 1 | 12 | 0.000 | 0.000 | 0.000 |
| SNP352 | 0.682 | 2 | 11 | 0.434 | 0.636 | 0.340 |
| SNP353 | 0.500 | 1 | 11 | 0.500 | 1.000 | 0.375 |
| SNP354 | 1.000 | 1 | 11 | 0.000 | 0.000 | 0.000 |
| SNP355 | 0.500 | 1 | 9 | 0.500 | 1.000 | 0.375 |
| SNP356 | 0.500 | 1 | 9 | 0.500 | 1.000 | 0.375 |
| SNP357 | 0.773 | 2 | 11 | 0.351 | 0.455 | 0.290 |
| SNP358 | 0.542 | 3 | 12 | 0.497 | 0.083 | 0.373 |
| SNP359 | 0.500 | 1 | 9 | 0.500 | 1.000 | 0.375 |
| SNP360 | 0.563 | 2 | 8 | 0.492 | 0.875 | 0.371 |
| SNP361 | 0.583 | 3 | 12 | 0.486 | 0.667 | 0.368 |
| SNP362 | 0.500 | 1 | 12 | 0.500 | 1.000 | 0.375 |
| SNP363 | 0.667 | 2 | 12 | 0.444 | 0.000 | 0.346 |
| SNP364 | 0.500 | 1 | 12 | 0.500 | 1.000 | 0.375 |
| SNP365 | 0.542 | 2 | 12 | 0.497 | 0.917 | 0.373 |
| SNP366 | 0.542 | 2 | 12 | 0.497 | 0.917 | 0.373 |
| SNP367 | 1.000 | 1 | 12 | 0.000 | 0.000 | 0.000 |
| SNP368 | 0.500 | 1 | 10 | 0.500 | 1.000 | 0.375 |
| SNP369 | 0.500 | 1 | 12 | 0.500 | 1.000 | 0.375 |
| SNP370 | 0.500 | 1 | 11 | 0.500 | 1.000 | 0.375 |
| SNP371 | 1.000 | 1 | 12 | 0.000 | 0.000 | 0.000 |
| SNP372 | 0.500 | 1 | 10 | 0.500 | 1.000 | 0.375 |
| SNP373 | 0.500 | 1 | 12 | 0.500 | 1.000 | 0.375 |
| SNP374 | 0.500 | 1 | 12 | 0.500 | 1.000 | 0.375 |
| SNP375 | 0.500 | 1 | 10 | 0.500 | 1.000 | 0.375 |
| SNP376 | 0.500 | 1 | 12 | 0.500 | 1.000 | 0.375 |
| SNP377 | 0.500 | 1 | 12 | 0.500 | 1.000 | 0.375 |
| SNP378 | 0.500 | 1 | 12 | 0.500 | 1.000 | 0.375 |
| SNP379 | 0.750 | 2 | 12 | 0.375 | 0.500 | 0.305 |
| SNP380 | 0.500 | 1 | 12 | 0.500 | 1.000 | 0.375 |
| SNP381 | 1.060 | 1 | 12 | 0.000 | 0.000 | 0.000 |
| SNP382 | 0.875 | 2 | 12 | 0.219 | 0.250 | 0.195 |
| SNP383 | 0.500 | 1 | 11 | 0.500 | 1.000 | 0.375 |
| SNP384 | 0.500 | 1 | 11 | 0.500 | 1.000 | 0.375 |
| Mean | 0.652 | 1.322 | 11.551 | 0.369 | 0.677 | 0.281 |

TABLE 11
SSRs and SSR Primer Sequences from Contigs and Annealing Temperatures

| | | | TM | | | Product | |
|---|---|---|---|---|---|---|---|
| SSR | Contig | Forward | Forward | Reverse | TM Reverse | Size | Repeat |
| 1 | Contig5 | ACACAACGACCCAGAAAACC | 59.867 | GACCCATCATCACCAGACTT | 57.314 | 475 | (CAT)4 |
| 2 | Contig8 | CCATCCTTCCTTCCTTCTTC | 57.77 | CAAGATATGCTTCGGTGAGG | 58.325 | 455 | (CAT)4 |
| 3 | Contig10 | AAGGAGAGGAGAAGAATGGG | 57.373 | GCCACTAGTAGGCTCAACCA | 57.98 | 435 | (TCT)5-(TCT)6- |
| | | | | | | | (ACA)4 |
| 4 | Contig26 | ACCACCTTCCCCTTAACAAC | 57.882 | GACCCTAACCACTTCCGAAT | 57.974 | 351 | (CAC)4 |
| 5 | Contig33 | TTAACCACTTAACCCTCCCC | 57.904 | CAGGAAGCGGAGACTCATAA | 58.048 | 429 | (CA)9 |
| 6 | Contig34 | TGTCGGCCTCTATCTCTCTG | 58.129 | ATGGCATCTTCACTCTCAGC | 57.969 | 180 | (AAG)4 |
| 7 | Contig39 | CCAACAAGTCCTCAAACACG | 59.183 | AATCAGAAGGGCAGAACTCC | 58.324 | 491 | (TAT)4 |
| 8 | Contig47 | ATAACTGGACTCACCAACCG | 57.514 | GATAGCACCGATGGAGAAGA | 57.834 | 360 | (GTC)4 |
| 9 | Contig69 | CAGTGTAGCCATCCTTCACC | 58.158 | TGTGAGGTTTGTGTGGAGTG | 58.063 | 107 | (CAACCA)5 |
| 10 | Contig74 | CTCTCTCCACATCAATGGC | 56.593 | GCAAGTGGGTCTTTACCCTT | 58.174 | 337 | (GAA)5 |
| 11 | Contig86 | CTAAATGGTCCTCCTGCTCA | 57.894 | GAAGCTCAACCTGCCTATGA | 58.048 | 498 | (CCG)4 |
| 12 | Contig93 | CCATCAATATCCACCGTACC | 57.61 | GTGTCAGATAGCGGCAGAAT | 57.921 | 202 | (CA)8 |
| 13 | Contig94 | GTCAAGTGGAGCTGTGGAGT | 57.844 | AAAGGTTGGGTAGATCCGTC | 57.974 | 454 | (TTC)4 |
| 14 | Contig104 | CTCAATCACTCACTCCACCA | 57.09 | CAGAGCTGATGCTAGCCAGT | 58.371 | 307 | (ACC)4 |
| 15 | Contig110 | CTCCGCCCATAAAACAAGAC | 59.569 | TCTGCCACACTTGACTCTGA | 58.03 | 283 | (TCT)4-(AC)6 |
| 16 | Contig132 | CCCACCACCTCCATTACATA | 58.145 | GCTCCAGATGGAGTCGTTTA | 57.893 | 293 | (ACA)4 |
| 17 | Contig147 | GATACTCCTGGGCTGTTTGA | 57.737 | CGATTCAGGGCAGTCTTCTA | 58.048 | 279 | (AAG)4 |
| 18 | Contig152 | ACACCTTTTCCCGTCCTTAG | 58.174 | GAAAGAGACAGCGGTTGAGA | 58.158 | 163 | (TCC)4 |
| 19 | Contig154 | CCCTTTCTTCTTCATCCCTC | 57.77 | GATGTTGACGATGACGAGTG | 57.573 | 134 | (CCA)4 |
| 20 | Contig155 | CTCCATCTTCCAAACCCACT | 58.989 | CCTCTTATCCCTCCCCATAA | 57.94 | 207 | (AAC)4 |
| 21 | Contig158 | TCAGGGCTAACTTCCTCTCA | 57.612 | GCTACAACTGGTGGGTGAAC | 58.058 | 355 | (CAC)5-(CCA)4 |
| 22 | Contig167 | GTTATGGACGCCGTTACTTG | 58.174 | CTCGCTTCGACAATCTCTGT | 58.187 | 226 | (TAA)5 |
| 23 | Contig180 | CCTTAACTCCACAGTTCCCC | 58.498 | AATCCGGTTGACTGGCTTAC | 59.056 | 438 | (ACAT)4 |
| 24 | Contig184 | TACTTTCAAGTCTCACCGCC | 57.951 | CAAGGGATAGCGTCACAGTT | 57.8.26 | 417 | (AAC)6 |
| 25 | Contig197 | GTGGTTGTAATCAAGGACGG | 57.923 | GAGACACAGGCACACAGATG | 57.714 | 385 | (TG)6 |
| 26 | Contig221 | CTTCTCTCTCCACATCCCAA | 57.78 | GATCCAACACCACCACTCTC | 57.886 | 419 | (GAG)4 |
| 27 | Contig232 | TGAGTTTCGTGGCTCTAAGG | 58.105 | CTCGTCGACTCAACGGTACT | 57.959 | 150 | (ATA)4 |
| 28 | Contig234 | CTGGCTGAAACAGGTGAAGT | 57.93 | GGCTACTTTCCTTTGCCCT | 58.398 | 440 | (GCT)4 |
| 29 | Contig236 | ATGGAGGGACCTTAAAACCC | 59.148 | GTCATTAACGACACGTTCCC | 57.922 | 225 | (TCT)4 |
| 30 | Contig238 | CCTCTGCCTACTCGTCAAAA | 58.105 | AGACGACGAACAAGACGAAG | 58.087 | 261 | (CA)6 |
| 31 | Contig245 | CGAGTTCCTTCAAGTCCTCA | 58.001 | CGACGAGTTAACGCTTCTGT | 58.187 | 187 | (AAT)5 |
| 32 | Contig265 | TCTGTAAGGCGGTTTCTGTC | 57.951 | ATAGAAGCAGGCATGGTGAG | 57.922 | 346 | (ACA)4 |
| 33 | Contig277 | GATCCTATTGCTCGTTGCC | 58.257 | ACTGCCCGATATCTCCTTTC | 58.214 | 371 | (ATT)4-(CA)6 |
| 34 | Contig294 | CTCCTATCAAAGGACGGAGA | 56.925 | ATCCTTGGACAGTAGCACCA | 58.158 | 459 | (TAA)6 |
| 35 | Contig295 | ACTGAGCCACCACTAACCAG | 57.801 | TAGACTTAGTGGCACCAGCC | 57.98 | 434 | (TTG)4 |
| 36 | Contig298 | ATTCAAAGGCCAGGGTAGAG | 58.266 | CGACTACAAACCGGACACTC | 58.215 | 297 | (TCA)4 |
| 37 | Contig312 | GCTGAGGAAGAGCATGGATA | 57.99 | CTCCACCATGTACCCGTT | 56.656 | 440 | (ACA)4 |
| 38 | Contig321 | CAGTGTAGCCATCCTTCACC | 58.158 | CACAGACCCAGCAAAGAACT | 57.93 | 202 | (ACAACC)4 |
| 39 | Contig335 | GAATGGGAGTGTGAATGGAG | 57.91 | TACTCCAAGATCAAGCCTGC | 58.048 | 308 | (AGA)4 |
| 40 | Contig339 | CAATTCTCTCTCCTCCCCAT | 58.118 | GAGGTGGTTTCTGGTTGTTG | 58.03 | 110 | (CTC)5 |
| 41 | Contig343 | ACAATAAGGAAGAGGGCCAC | 58.121 | GGCTGAAGTCTTGTGCTCAT | 58.028 | 365 | (TGG)5 |
| 42 | Contig349 | CCGTCTCATTGACCTACCTT | 56.702 | ACCTGGCCCCACTTCTATAC | 58.001 | 427 | (GAC)5 |
| 43 | Contig353 | CCTCCCTCCTTCTTCTGGTA | 58.323 | CAGCCTTGGATCTTGTGAAC | 58.287 | 347 | (TAC)4 |
| 44 | Contig357 | CAGACGTCATTGCTGAAGTG | 57.977 | ACTGGAAGTCTGCCCTTTTC | 58.377 | 405 | (CCA)4 |
| 45 | Contig363 | AAATATCTCCCCAACCCCTC | 59.104 | TCAGGCTTAGTTGTTCCAGG | 57.952 | 356 | (CT)6 |
| 46 | Contig366 | ACGAAGCAGTTTACCACCAC | 57.729 | CACCGTATCTTCGCACTTCT | 57.98 | 161 | (AGG)4 |
| 47 | Contig370 | CCATGGTTAGGACTCAGGTC | 57.001 | CCCAAGTTCAGACCGTAGAT | 56.702 | 133 | (TC)6 |
| 48 | Contig371 | CGTATCACTTTCAACCCGAC | 58.076 | TCTCGCCTTCTCTATTGGTG | 58.048 | 408 | (GAG)4 |
| 49 | Contig375 | ATCCTGTTCTTGACGAGCTG | 58.029 | AGCTCGTTACGTTGGTTGTC | 57.883 | 454 | (TGA)4 |
| 50 | Contig380 | CCATCGATCATCAGTTAGGG | 57.962 | GTTGCTCCACCAAGTACACC | 58.058 | 365 | (TCG)4 |
| 51 | Contig382 | GCCTGAAATCAAGTGTGTCC | 58.129 | CATAGGTTCCATGGGTTACG | 57.823 | 478 | (TTC)4 |
| 52 | Contig390 | CTCAAGCCCTACATCTTCCC | 58.741 | TCTTAGGAGCACGTCCAAAC | 57.951 | 164 | (TAA)4-(TGCC)4 |
| 53 | Contig390 | GGTGGAGACCCTGAGAAGTT | 58.159 | CATACCAGAACCTTGGCATC | 58.019 | 396 | (TGG)4 |
| 54 | Contig392 | CAACACTCAACACCTCGTGA | 58.232 | TCATACTGCATACCCGAACC | 58.45 | 340 | (GAT)4 |
| 55 | Contig393 | GTATTGTTCAAGTGTGGGGG | 57.768 | GGGTGACATATTTGGGTTGG | 59.9 | 497 | (TA)6 |
| 56 | Contig395 | GCAACAACAATGGCAACC | 58.458 | AGGTGGTCCACTATGCTCTG | 57.74 | 386 | (TCC)5 |
| 57 | Contig401 | TTACCCACTGACGTCATCCT | 58 | TGTATCCGTCACCTCCTCAT | 57.94 | 343 | (ATA)6 |
| 58 | Contig403 | GTTTCTTCAAGCGTCCTC | 58.802 | GCTCTTCACCCAGTCAAAGA | 58 | 490 | (GAT)5 |
| 59 | Contig414 | ACGCTTCTTCAGTGCTTCTG | 58.398 | CCTGTTCCCAGCTTTTAACC | 58.694 | 495 | (AC)9 |
| 60 | Contig421 | AACCGTCTAAAACCCGACTC | 58.174 | GTTACCGGAGAGGATGGATT | 57.918 | 213 | (ACC)4 |
| 61 | Contig422 | CTCACTTGTATCATTGCCCC | 58.019 | GTCAAGCCCATCTGTGACTC | 58.216 | 432 | (AAC)6-(TGA)4 |
| 62 | Contig467 | GCAGGCCGTATCACAAATAC | 58.12 | TAGGTGGATAGAACACCGGA | 58.02 | 241 | (CCA)4 |
| 63 | Contig468 | AAACCCTAGACACCGAAACC | 58.029 | CGTCACCAAAACCCTACAAC | 57.98 | 435 | (GCC)5 |
| 64 | Contig468 | GTTGTAGGGTTTTGGTGACG | 57.98 | GACCAATAGCAAGAGCAAGC | 57.725 | 243 | (GGA)4 |
| 65 | Contig471 | ACACCCCTCTCTCTCTTGCT | 58.06 | AATATGGGCTATCAGAGGGC | 58.109 | 245 | (TTG)5 |
| 66 | Contig473 | ACAACACTGTGTAATGGCCC | 58.343 | CCCCATATCCATAACTTCCC | 58.057 | 342 | (ACC)5 |
| 67 | Contig482 | TCTCATCTCAATGCTCCTCC | 57.879 | GCATCTGACGCTTGTAGCTC | 58.78 | 325 | (AAT)4-(TCC)5 |
| 68 | Contig484 | AACAATACCTCGCACTCTGC | 57.979 | GTTGGCTATGATTGGACGAC | 58.019 | 411 | (AAC)6 |
| 69 | Contig487 | ATCTTGCCTCTCTAAAGCCC | 57.639 | CGGAAGCACGATGAACTAGA | 59.024 | 327 | (AG)6 |
| 70 | Contig496 | TACCCTCCTCAGACCCTACC | 58.05 | AGCCCCACTTACCATGATCT | 58.473 | 143 | (ATG)6 |
| 71 | Contig498 | CTCAAGCGTATGAGTTGGCT | 58.132 | CCGGGACTTTTTACTCCTTC | 57.787 | 229 | (GAT)4 |
| 72 | Contig525 | CTGTTTCACCGATTCTGACC | 58.129 | ATGCACCTAAGTCCACAGGA | 58.158 | 473 | (TAA)5 |
| 73 | Contig534 | GACCAACACCCCCTTTTC | 57.718 | GACCAGCCAACACAAAGAAC | 38.187 | 294 | (TC)8 |
| 74 | Contig539 | AACAGCTGGAGACCCAAGTA | 57.387 | TAACCTACCAACTCGCTTGC | 58.054 | 278 | (GGT)4 |
| 75 | Contig546 | ACCCTAGATCCTCGGTTCAC | 18.049 | GCTTCGACAGGCAGTTATGT | 57.979 | 440 | (CCA)4 |
| 76 | Contig548 | GACGATGAGTTCAGAAACGG | 58.287 | GACACGTCATTTCCACCTTC | 57.97 | 370 | (TAC)5 |
| 77 | Contig570 | ATCCTCCCAAGAAGATGACC | 57.964 | TCTCTCCTGCAAGAAGCTGT | 57.9 | 230 | (AAC)4 |
| 78 | Contig577 | AAGAAGAGGAGCCTGGGTTA | 58.002 | GAAGGAAAAAGAGGCGTACC | 57.929 | 253 | (TCT)4 |
| 79 | Contig582 | TGGCGAAAGAAGAAGAGG | 56.587 | GAGAGGATGGCTATAAGGGG | 57.702 | 466 | (ATG)5 |
| 80 | Contig582 | CATGCTACCCCTTATAGCCA | 57.754 | GTTTTGTAGGACAAGTCCGC | 58.433 | 177 | (AACCGA)4 |
| 81 | Contig591 | TGGAGTCTCCTGCTTCAAAC | 58 | GTGTTGGTGTGTGCTATCCA | 57.945 | 335 | (CAG)4 |
| 82 | Contig592 | CTACTGGTGGAGAAGGATGC | 57.323 | ACCTTCACTCACAAAGCTCG | 58.087 | 281 | (CCA)5 |
| 83 | Contig606 | GTCATCTCAACCACCACCAT | 58.186 | GGATGACAAGAGGACGTGAG | 58.216 | 242 | (CAC)4 |
| 84 | Contig612 | ACAAACCCTAACTGGACCGT | 58.429 | TTCACGCTCGAGTCTTAACC | 58.104 | 157 | (CCA)4 |
| 85 | Contig619 | TCTCTCTCCTCACCTACGTCAT | 58.01 | AAATCCTCAAGACTGGTCCC | 58.021 | 390 | (AAT)4 |
| 86 | Contig629 | TCAGGGTAAGGAGGAAGGAC | 58.173 | CGAGAATCTTCCCCGAGTAT | 58.214 | 315 | (TGA)4 |
| 87 | Contig629 | AGATTCTCGAGTGTGCTTGG | 58.029 | GACCTCGCTCCCTAATTACA | 56.909 | 318 | (CCA)4 |
| 88 | Contig632 | CTGCCTGCGTTTTCTCAT | 57.474 | GGAGATTCTTGGAGGAAGGA | 58.242 | 398 | (TTA)4 |
| 89 | Contig635 | GAAAGCGGATCACACAGC | 57.817 | TTCTCCAGAACATCCGAGTG | 58.8 | 314 | (CTAT)4 |
| 90 | Contig639 | TTCAACCTTAGGTCCAGACG | 57.799 | CTAGCACCAGCCGTAACAAT | 57.933 | 327 | (CAC)4 |
| 91 | Contig645 | GTCACCACTTCCTCCAAATG | 57.971 | TGGTGCTGATGATACAGTGG | 58.05 | 445 | (TAC)4 |
| 92 | Contig647 | TTACGGACGGAGACAAGAAG | 57.952 | TCATCATAGTGCTTGCGG | 56.735 | 259 | (CAC)4 |
| 93 | Contig663 | CCAAATCAACACCACCACC | 59.641 | CACTGCTCTTACGGATTGCT | 58.132 | 229 | (TCAC)4 |
| 94 | Contig686 | CTCATTCACCTCCCACCTAA | 57.582 | TACTTGACCTACTGGCAGGC | 57.98 | 468 | (AAT)4 |
| 95 | Contig711 | ACGAAGATGCACTCCTGAAG | 58.029 | AGCGGTCATGAGTCGATTAG | 57.922 | 315 | (ATG)4 |
| 96 | Contig716 | CCTGTTGCTTCTAGGGTGAA | 57.952 | GAACGGGTCTCCAAGGTTAT | 57.974 | 306 | (AGC)6-(TGA)5 |
| 97 | Contig717 | CCACCTCCATCTTAACCCAT | 58.715 | CATAAGCCGAAGTCACCCTA | 57.85 | 373 | (ATA)4 |
| 98 | Contig720 | TTTGTAACCCTCACACACCC | 58.315 | CAGTACTGACGGCGACTTCT | 58.116 | 224 | (AGG)4 |
| 99 | Contig722 | CTCACTCTCTCTCCAGAAGCA | 57.464 | GCACAGCTCCACTCATCTTT | 58.028 | 260 | (GAT)4 |
| 100 | Contig727 | CCCATGTACCGATCAAAGAG | 58.02 | AGGATAACCATAGCCCGGT | 58.347 | 326 | (CAA)4 |
| 101 | Contig728 | GACGGAAATAAACCGTCGTC | 59.434 | GAGAAGTTTTCTCGCGTGTG | 58.65 | 430 | (GGT)4 |
| 102 | Contig734 | CACGTTCCTCCTACCGTAAA | 57.758 | CTAGAACGTCGGAAGGTTGA | 57.952 | 108 | (CTA)5 |
| 103 | Contig737 | GAACGTTGACAAGGTGGAAC | 58.029 | TCGTGTCACGTGGAACATAC | 57.945 | 242 | (TGG)4 |
| 104 | Contig739 | ACGGTATCGTCCTTTCCTTC | 58.121 | AGCATCTCAACGATGCCTAC | 57.921 | 404 | (AGA)5 |
| 105 | Contig744 | TAGTACCACTCCACAAGCCC | 57.671 | TGATGACACCCTTGACAGTG | 57.999 | 204 | (TCC)4 |
| 106 | Contig747 | GACACTCTCTCTCATCCCCA | 57.686 | GCCTCTGCTCTCCTCTTTCT | 57.953 | 302 | (TC)6-(CAC)4 |
| 107 | Contig750 | GACCCATACACAGGCGTTAG | 18.104 | CTGTGAAGCCACATCAACAC | 57.64 | 274 | (TGA)4 |
| 108 | Contig754 | CTCCCTCACTCTTCGAAACA | 58.001 | GCCGACGTAACAGAACAGAT | 57.824 | 369 | (CT)8 |
| 109 | Contig757 | AGCAAATTGGGGCTGTGT | 59.09 | GAGGCCCATATTCTCCAATC | 58.402 | 484 | (ATT)4 |
| 110 | Contig762 | GTTGCTGTCGAAAAGCTCC | 58.585 | CCGAGTGAGCTGTGGTTAGT | 57.959 | 179 | (TC)8 |
| 111 | Contig780 | CTCTCACTCCTCTCAGCAGC | 57.979 | TGTTAAACTCACCTCCGGTC | 57.64 | 439 | (ATG)5 |
| 112 | Contig808 | CCCCTTTCATCTCTCTAGCC | 57.946 | CCGAGCTCTTTCCTAGCTTT | 57.983 | 406 | (AG)8 |
| 113 | Contig816 | GGCCCAGAATCAAGGAAGTA | 59.126 | GGATCCTCACATTCATCTGC | 58.01 | 354 | (GTG)4 |
| 114 | Contig826 | CCCCAATCAAAGCCCTAA | 58.492 | CAAACACACCTTGTTCCTCC | 58.03 | 371 | (TTC)5-(ACC)4 |
| 115 | Contig829 | GTGAAAGGGATGTGTTGGAG | 57.971 | CCCAACCTCATCTTCTCCTC | 58.664 | 122 | (GAT)4 |
| 116 | Contig830 | CAAGGAAGTGGTTGAAGGAG | 57.36 | GTAGGACGCGTAACGATCTC | 57.449 | 298 | (AAG)4 |
| 117 | Contig836 | AATAAGACCCACACCCAACC | 58.62 | GTGACGATGGATGTAAAGGC | 58.019 | 175 | (TCA)5-(TTA)5 |
| 118 | Contig849 | GGTAGGTCGCCTAAGAGAGG | 58.029 | CAGACCACACTATTTCCCCC | 59.259 | 457 | (CTT)4 |
| 119 | Contig854 | GTAACCACATGGACGATGCT | 58.444 | GCAGCTGAATCTCCAACACT | 58.028 | 412 | (CATTCA)4- |
| | | | | | | | (TCA)5-(ATG)4 |
| 120 | Contig868 | CCCTACACTCCACCACACTC | 57.979 | TGATCGGAGGAGGTAGTCAG | 57.81 | 483 | (CCA)5 |
| 121 | Contig880 | GGGAAGGAAAGAAAGTGAGG | 57.827 | GTCTTCAGGATGCACCATTC | 58.07 | 332 | (TAA)4-(TCC)6 |
| 122 | Contig880 | TACTCAGGTGATGGTGGGTT | 57.84 | GTCTTCAGGATGCACCATTC | 58.07 | 241 | (TGT)4 |
| 123 | Contig904 | ACCCTAGGGGACGATTGTAG | 58.002 | TTCCCTTTAGGCTGTGAGTG | 57.952 | 257 | (ATA)4 |
| 124 | Contig912 | GTCATCTCCAACCACCGTA | 56.809 | GTGTGAGAGGGAGATTGTGG | 58.052 | 207 | (CA)7 |
| 125 | Contig919 | TGCTCCTACAGAGCCAAAAG | 58.256 | CCAACCAGATCCACAAGAAG | 58.13 | 323 | (TGCA)4 |
| 126 | Contig932 | CCCTACCACCTGTTGTCAAG | 58.059 | AGATCCACACAAGCTCCAAG | 57.87 | 328 | (CCA)4 |
| 127 | Contig955 | TGAGTTTCGTGGCTCTAAGG | 58.105 | GAACGGTAGACGGAGCACTA | 57.98 | 224 | (ATA)4 |
| 128 | Contig964 | CACCAAGCTTTTCTGGTCC | 58.272 | AGATGGAGGGGTATGAGAGG | 57.993 | 142 | (AG)8 |
| 129 | Contig974 | CTATTGATGCCCCTCTCTCTC | 57.96 | AAAACCCCACATCTCTCTCC | 58.021 | 111 | (TC)6 |
| 130 | Contig981 | GATTACTCGTTCAACGGTCG | 58.228 | AGGAGGAAAAAGGGGGTACT | 58.074 | 327 | (TAG)6 |
| 131 | Contig1011 | AGAGAAATCTGCACCACTCG | 58.029 | CTCCTTCTTCTTCGCCTTCT | 57.882 | 465 | (ATG)4 |
| 132 | Contig1048 | GCCGCCTCCACTAGATAAA | 57.929 | AGCATCTTAGCCTCAGGGTT | 58.028 | 283 | (GAA)4 |
| 133 | Contig1051 | CGTCTCCTATCATCCGATTG | 58.117 | GGAGAAGACGATAAGCACCA | 57.893 | 352 | (TTA)4 |
| 134 | Contig1053 | CCGAGTAACCCAACATTCTG | 58.077 | AAGATCCCAAGAACCCAGTC | 58.021 | 397 | (TGA)5 |
| 135 | Contig1054 | CCCTATGTTTCCGATCCTCT | 58.067 | ATCAGCGCTAGATGCTTCAC | 58.229 | 203 | (TCA)4 |
| 136 | Contig1063 | GCAAAACAGATCTACCACCG | 58.228 | CGACTAATGGGTCGAATGAG | 58.173 | 345 | (CTTCAC)6- |
| | | | | | | | (CTC)4-(ACC)4 |
| 137 | Contig1068 | AACTCCAAACTCCACCTCTC | 55.784 | GCTCCAGATGGAGTCGTTTA | 57.893 | 205 | (ACA)4 |
| 138 | Contig1069 | CCCCTAGGGTTTCATTCACT | 57.975 | CCTGTTGTCCTGCTTGTTCT | 57.93 | 203 | (AC)6 |
| 139 | Contig1081 | AAAGCAGCAACACTCTCCC | 58.014 | ATCTCAACCTCCTCAAACCC | 58.021 | 226 | (TAA)4-(ATC)5 |
| 140 | Contig1095 | TGGCCTCTTCTTCTAGGGTT | 58.002 | CACGTGTTCTTTCTCAGCCT | 58.087 | 101 | (TTC)4 |
| 141 | Contig1098 | GCAACCCATCTCACACATAC | 56.335 | CACACACGTGTACTCCTCCA | 58.094 | 416 | (AAT)4-(TAA)5 |
| 142 | Contig1098 | AGCTGTTGGAGGAGTACACG | 57.959 | CTGCTGGTACCGACATTCTT | 57.826 | 293 | (TGG)4 |
| 143 | Contig1107 | GGCTATCCACAAATGTACGC | 58.12 | GCATAGTGGCTCCTGTTGTC | 58.316 | 206 | (CCA)4 |
| 144 | Contig1117 | CACCCTCTTCATCGCAAAC | 59.23 | GTCATCCTAAACGCTGTCGT | 57.824 | 207 | (GAA)4 |
| 145 | Contig1123 | GACTCCACCAACCAACACTC | 57.948 | CAGTTGGACCACCAGTAACC | 57.901 | 395 | (AAT)5 |
| 146 | Contig1126 | CCGCTAACAATCACTTCACC | 58.228 | TACATGTGTAACTGTGGGCG | 58.057 | 419 | (ATA)5 |
| 147 | Contig1138 | CGTCTTGCAGGATGAAGTCT | 58.029 | GAGACCCGACAGATTGATTG | 58.071 | 464 | (ATT)4 |
| 148 | Contig1139 | CTCTTCTTCCATCTCACCCA | 57.78 | CCGACTCCTTCTTGAACAGA | 58.001 | 229 | (GAA)4 |
| 149 | Contig1146 | CCGAGTGAGTAGACCCAGAA | 57.871 | ACTGAGTCAACCCGGTCATA | 58 | 167 | (TAT)4 |
| 150 | Contig1146 | GTATGACCGGGTTGACTCAG | 58 | TGGTTCACACCCTCTGTTCT | 58.113 | 383 | (ATGGTC)6 |
| 151 | Contig1158 | GGGAGGATTAAAGGATCTCG | 57.675 | CGCTCAACCAGTTTAGCATC | 58.529 | 414 | (TC)8 |
| 152 | Contig1173 | CGGACCAAACGAAGAACA | 58.156 | CCACCTGATTATTCGACGAC | 58.019 | 221 | (TCT)4 |
| 153 | Contig1175 | CAACCCCAATTATACCCCTC | 58.11 | TAACACGTGGGTAACACCCT | 57.856 | 338 | (CAG)4 |
| 154 | Contig1189 | ACTCAGGCATCTGATCATGG | 58.616 | TCAGTTTCTTGCCCTGCTAC | 58.104 | 279 | (TCT)4 |
| 155 | Contig1197 | ATACAGGTCACTGGCTCTCG | 57.9 | CCTAGGAGAACCCCACTTGT | 58.106 | 403 | (GAA)5 |
| 156 | Contig1199 | ACCTAAACACACCGAGTCCA | 58.059 | GCAACGAGTTCAGACCACTC | 58.436 | 213 | (TCC)4 |
| 157 | Contig1202 | TAAGACGGAGCACAAGGAAG | 58.105 | ATAACAGCTCTGCCTTCACG | 58.132 | 196 | (AGC)4 |
| 158 | Contig1211 | TTACTTAAGCCCTGTCGTGG | 57.907 | GGATCAGAACCTTCATGCAC | 58.07 | 216 | (CAGGTT)4 |
| 159 | Contig1231 | TGCTCACCAGGTTAGGTGTT | 58.215 | GCTCTCAAGTTACTGCAGCC | 57.853 | 314 | (TTA)4 |
| 160 | Contig1233 | TGTGTGCTCTACGAACCAGA | 57.977 | CCTCTTAACCAGAGGCACAA | 57.952 | 498 | (ATG)5 |
| 161 | Contig1234 | GAACATCTGCAAACCCTAGC | 57.419 | CACCACTGTTATCAGCACCA | 58.112 | 454 | (CAT)4 |
| 162 | Contig1248 | GTTCTGGTGTTGATGGGAGA | 58.482 | ATCTCCACCATCCTTCCTCT | 57.555 | 480 | (ATG)5 |
| 163 | Contig1249 | GATGGCTATGTCTATGGCGA | 58.699 | GCTTCTCTAGTTCGCAGTCG | 58.008 | 284 | (TCC)4 |
| 164 | Contig1255 | GGAATCTCTGACCAATCCCT | 57.964 | CCACACACCTTCACAACTCA | 58.063 | 442 | (TAT)4 |
| 165 | Contig1260 | CAACCCTTGTGACCCACTAC | 57.901 | ACAGCCACCACTCTAACCAC | 57.639 | 384 | (GA)8 |
| 166 | Contig1264 | TACACCTTCACCCACTCCAT | 57.84 | AAACATAGACTCGAACCCCC | 57.974 | 467 | (AAT)4 |
| 167 | Contig1276 | ACCACCACATACACCACACA | 58.062 | CGGGTTTGCTGACTTACACT | 57.884 | 450 | (TAT)4 |
| 168 | Contig1277 | GTTATCCCATGGAAAGACCC | 58.16 | GAACCTCTTAACCGCTCTCC | 58.001 | 351 | (ACC)5 |
| 169 | Contig1278 | GGAAGTCTTACGTGTCACCG | 58.215 | GATTCAGGATTCAGGTCACG | 58.071 | 190 | (TTA)4 |
| 170 | Contig1279 | CTGGATGTTGAGATCCGACT | 57.647 | GTTACACCCAGGTTGGTCTG | 57.901 | 228 | (CAA)4 |
| 171 | Contig1279 | CGGAGTGAAGCTACAAGGAA | 58.105 | CAGACAACCCTGTTGAAACC | 58.03 | 194 | (AAT)5 |
| 172 | Contig1286 | GAGTTGGAGGGGTTCTTGTT | 58.078 | GTCTTCAGGATGCACCATTC | 58.07 | 231 | (CTC)4 |
| 173 | Contig1303 | CACGTTGTAAGCACGTCATC | 57.764 | TAGGGCTTGTGTGACCACTT | 58.215 | 495 | (TGTA)4 |
| 174 | Contig1307 | CCTTGCTCCAGTGTCTTGTA | 56.46 | TAAGTTAGCCCGTTGTCGAG | 58.054 | 444 | (CAG)4 |
| 175 | Contig1307 | TCGACAACGGGCTAACTTAC | 57.906 | CTTGTAGTGCATTTCCCACC | 58.076 | 472 | (CCT)4 |
| 176 | Contig1313 | CGAAGGAGAAGACTCCACAA | 58.001 | CACCTTCCTGAATGATCTGG | 58.071 | 443 | (CAA)5 |
| 177 | Contig1316 | CATCGATCGGAACTGCTAAC | 58.326 | CTTCCTTCTTCACTGGCGTA | 58.105 | 195 | (AAC)5 |
| 178 | Contig1318 | TTTCTATCGAGGGAAGGAGG | 58.333 | GCCTTCTGTCAAACACTGCT | 58.087 | 425 | (GAT)4 |
| 179 | Contig1333 | GGGTACAGAAACCAACCGAT | 58.766 | AGCACTGACGACTAACCCAC | 57.8 | 142 | (CAA)5 |
| 180 | Contig1354 | AGAAGAGCCTCCACAAACCT | 57.982 | CATCACCGTCATCACCAGTA | 57.883 | 262 | (AAG)4 |
| 181 | Contig1358 | ACTCTTCGCCTTTGATCCTC | 58.473 | CTACAGCCAAGGAATGTGCT | 57.98 | 377 | (CTT)4 |
| 182 | Contig1376 | TGACTGTATGGAGACGGAGC | 58.83 | CATCGATACACCGATCTTCC | 57.961 | 119 | (CGC)4 |
| 183 | Contig1379 | TCTTCCCCTCCATATCACAC | 57.357 | TCTGAGAACAGAGGACAGGG | 57.919 | 123 | (ATT)5 |
| 184 | Contig1381 | AAAAGAGGAACGAGGAGTGG | 58.376 | TGTCCTGAGCCTCATACTCC | 57.809 | 132 | (GAA)4 |
| 185 | Contig1384 | ACACAGGATCAACCCAGCTA | 58.158 | GAACAGGAGGCTGTTGAAGA | 58 | 289 | (CA)7 |
| 186 | Contig1394 | AGAGGCTAAAGAACCCGAAG | 57.697 | CCAACCAGATCCACAAGAAG | 58.13 | 155 | (TGCA)4 |
| 187 | Contig1397 | GGAACCTCAGGGTTACGAAT | 57.974 | ACCAGAGTCTCCCTCTGCTT | 58.06 | 379 | (GAA)5 |
| 188 | Contig1403 | CACCCTTACAACCAGACACC | 57.901 | GTGCTCCATATAATCCCCCT | 57.82 | 339 | (CGA)5 |
| 189 | Contig1407 | GCTTACCGTCATTCACTGCT | 57.979 | GTAGGTGTAATGTGGGGGTG | 57.641 | 444 | (CAC)4 |
| 190 | Contig1426 | GGTAACACCACCATCAGGAG | 57.84 | CAAGTGTAACCAGGAGGTGG | 58.059 | 324 | (CCA)4 |
| 191 | Contig1459 | CATAACCCAAGTTCTGGTGG | 57.924 | GAGCTGAATCTTTCGCTCTG | 57.922 | 377 | (CTT)5 |
| 192 | Contig1481 | GCAGATGGTCAGATTCATCC | 58.01 | CAGTCGGAGTAAACCCTGAA | 57.799 | 355 | (AGA)4 |
| 193 | Contig1493 | CCGATCCTTCTCTCTCCTTC | 57.993 | CTCTTGAGAAGCATCACGGT | 58.029 | 278 | (AAG)4 |
| 194 | Contig1494 | GAAGAAAGAGGTAGACCGGG | 57.854 | ACCCCAACACGACTGTTTAC | 57.417 | 245 | (TAA)4 |
| 195 | Contig1501 | ATTCAGCGTCTGCTAGCCT | 57.809 | GATGGTGATCTCATGGAAGC | 58.01 | 328 | (TTC)4 |
| 196 | Contig1503 | CTCTAACTTTGGACGACGAC | 55.024 | TGAACATGAGCCTCCTTAGC | 58.048 | 334 | (CTT)4 |
| 197 | Contig1503 | GCTAAGGAGGCTCATGTTCA | 58.048 | ACATTCATCCCCTCCTCTTC | 57.964 | 260 | (TCT)4 |
| 198 | Contig1525 | TGGACTGAGGGCATTATAGC | 57.791 | AAACCACTTCTCACAGCAGC | 58.087 | 181 | (CTG)4 |

TABLE 12
SSRs and SSR Primer Sequences from Singletons and Annealing Temperatures.

| Sequence ID | Contig |
|---|---|
| 1 | gi\|294712248\|gb\|GW780481.1\|GW780481_PBC024E10_04909_Parthenium_ |
| | argentatum_cold_acclimated_bark_cDNA_library_(PBC)_Parthenium_argentatum_ |
| | cDNA_clone_PBC024E10,_mRNA_sequence |
| 2 | gi\|294712235\|gb\|GW780468.1\|GW780468_PBC024D07_04896_Parthenium_ |
| | argentatum_cold_acclimated_bark_cDNA_library_(PBC)_Parthenium_argentatum_ |
| | cDNA_clone_PBC024D07,_mRNA_sequence |
| 3 | gi\|294712234\|gb\|GW780467.1\|GW780467_PBC024D06_04895_Parthenium_ |
| | argentatum_cold_acclimated_bark_cDNA_library_(PBC)_Parthenium_argentatum_ |
| | cDNA_clone_PBC024D06,_mRNA_sequence |
| 4 | gi\|294712199\|gb\|GW780432.1\|GW780432_PBC024A07_04860_Parthenium_ |
| | argentatum_cold_acclimated_bark_cDNA_library_(PBC)_Parthenium_argentatum_ |
| | cDNA_clone_PBC024A07,_mRNA_sequence |
| 5 | gi\|294711956\|gb\|GW780431.1\|GW780431_PBC024A06_04859_Parthenium_ |
| | argentatum_cold_acclimated_bark_cDNA_library_(PBC)_Parthenium_argentatum_ |
| | cDNA_clone_PBC024A06,_mRNA_sequence |
| 6 | gi\|294711943\|gb\|GW780418.1\|GW780418_PBC023H05_04806_Parthenium_ |
| | argentatum_cold_acclimated_bark_cDNA_library_(PBC)_Parthenium_argentatum_ |
| | cDNA_clone_PBC023H05,_mRNA_sequence |
| 7 | gi\|294711919\|gb\|GW780394.1\|GW780394_PBC023F05_04822_Parthenium_ |
| | argentatum_cold_acclimated_bark_cDNA_library_(PBC)_Parthenium_argentatum_ |
| | cDNA_clone_PBC023F05,_mRNA_sequence |
| 8 | gi\|294711892\|gb\|GW780367.1\|GW780367_PBC023D02_04795_Parthenium_ |
| | argentatum_cold_acclimated_bark_cDNA_library_(PBC)_Parthenium_argentatum_ |
| | cDNA_clone_PBC023D02,_mRNA_sequence |
| 9 | gi\|294711892\|gb\|GW780367.1\|GW780367_PBC023D02_04795_Parthenium_ |
| | argentatum_cold_acclimated_bark_cDNA_library_(PBC)_Parthenium_argentatum_ |
| | cDNA_clone_PBC023D02,_mRNA_sequence |
| 10 | gi\|294711884\|gb\|GW780359.1\|GW780359_PBC023C05_04787_Parthenium_ |
| | argentatum_cold_acclimated_bark_cDNA_library_(PBC)_Parthenium_argentatum_ |
| | cDNA_clone_PBC023C05,_mRNA_sequence |
| 11 | gi\|294711624\|gb\|GW780341.1\|GW780341_PBC023A10_04_769_Parthenium_ |
| | argentatum_cold_acclimated_bark_cDNA_library_(PBC)_Parthenium_argentatum_ |
| | cDNA_clone_PBC023A10,_mRNA_sequence |
| 12 | gi\|294711616\|gb\|GW780333.1\|GW780333_PBC023A02_04761_Parthenium_ |
| | argentatum_cold_acclimated_bark_cDNA_library_(PBC)_Parthenium_argentatum_ |
| | cDNA_clone_PBC023A02,_mRNA_sequence |
| 13 | gi\|294711596\|gb\|GW780313.1\|GW780313_PBC022G05_04741_Parthenium_ |
| | argentatum_cold_acclimated_bark_cDNA_library_(PBC)_Parthenium_argentatum_ |
| | cDNA_clone_PBC022G05,_mRNA_sequence |
| 14 | gi\|294711564\|gb\|GW780281.1\|GW780281_PBC022D08_04709_Parthenium_ |
| | argentatum_cold_acclimated_bark_cDNA_library_(PBC)_Parthenium_argentatum_ |
| | cDNA_clone_PBC022D08,_mRNA_sequence |
| 15 | gi\|294711309\|gb\|GW780268.1\|GW780268_PBC022C05_04696_Parthenium_ |
| | argentatum_cold_acclimated_bark_cDNA_library_(PBC)_Parthenium_argentatum_ |
| | cDNA_clone_PBC022C05,_mRNA_sequence |
| 16 | gi\|294711292\|gb\|GW780251.1\|GW780251_PBC022A12_04679_Parthenium_ |
| | argentatum_cold_acclimated_bark_cDNA_library_(PBC)_Parthenium_argentatum_ |
| | cDNA_clone_PBC022A12,_mRNA_sequence |
| 17 | gi\|294711289\|gb\|GW780248.1\|GW780248_PBC022A09_04676_Parthenium_ |
| | argentatum_cold_acclimated_bark_cDNA_library_(PBC)_Parthenium_argentatum_ |
| | cDNA_clone_PBC022A09,_mRNA_sequence |
| 18 | gi\|294711254\|gb\|GW780213.1\|GW780213_PBC021F10_04641_Parthenium_ |
| | argentatum_cold_acclimated_bark_cDNA_library_(PBC)_Parthenium_argentatum_ |
| | cDNA_clone_PBC021F10,_mRNA_sequence |
| 19 | gi\|294711242\|gb\|GW780201.1\|GW780201_PBC021E10_04629_Parthenium_ |
| | argentatum_cold_acclimated_bark_cDNA_library_(PBC)_Parthenium_argentatum_ |
| | cDNA_clone_PBC021E10,_mRNA_sequence |
| 20 | gi\|294710969\|gb\|GW780170.1\|GW780170_PBC021C02_04598_Parthenium_ |
| | argentatum_cold_acclimated_bark_cDNA_library_(PBC)_Parthenium_argentatum_ |
| | cDNA_clone_PBC021C02,_mRNA_sequence |
| 21 | gi\|294710966\|gb\|GW780167.1\|GW780167_PBC021B11_04595_Parthenium_ |
| | argentatum_cold_acclimated_bark_cDNA_library_(PBC)_Parthenium_argentatum_ |
| | cDNA_clone_PBC021B11,_mRNA_sequence |
| 22 | gi\|294710923\|gb\|GW780124.1\|GW780124_PBC020G04_04552_Parthenium_ |
| | argentatum_cold_acclimated_bark_cDNA_library_(PBC)_Parthenium_argentatum_ |
| | cDNA_clone_PBC020G04,_mRNA_sequence |
| 23 | gi\|294710668\|gb\|GW780111.1\|GW780111_PBC020F03_04539_Parthenium_ |
| | argentatum_cold_acclimated_bark_cDNA_library_(PBC)_Parthenium_argentatum_ |
| | cDNA_clone_PBC020F03,_mRNA_sequence |
| 24 | gi\|294710659\|gb\|GW780102.1\|GW780102_PBC020E05_04530_Parthenium_ |
| | argentatum_cold_acclimated_bark_cDNA_library_(PBC)_Parthenium_argentatum_ |
| | cDNA_clone_PBC020E05,_mRNA_sequence |
| 25 | gi\|294710641\|gb\|GW780084.1\|GW780084_PBC020C11_04512_Parthenium_ |
| | argentatum_cold_acclimated_bark_cDNA_library_(PBC)_Parthenium_argentatum_ |
| | cDNA_clone_PBC020C11,_mRNA_sequence |
| 26 | gi\|294710624\|gb\|GW780067.1\|GW780067_PBC020B05_04495_Parthenium_ |
| | argentatum_cold_acclimated_bark_cDNA_library_(PBC)_Parthenium_argentatum_ |
| | cDNA_clone_PBC020B05,_mRNA_sequence |
| 27 | gi\|294710602\|gb\|GW780045.1\|GW780045_PBC019H04_04473_Parthenium_ |
| | argentatum_cold_acclimated_bark_cDNA_library_(PBC)_Parthenium_argentatum_ |
| | cDNA_clone_PBC019H04,,_mRNA_sequence |
| 28 | gi\|294710597\|gb\|GW780040.1\|GW780040_PBC019G11_04468_Parthenium_ |
| | argentatum_cold_acclimated_bark_cDNA_library_(PBC)_Parthenium_argentatum_ |
| | cDNA_clone_PBC019G11,_mRNA_sequence |
| 29 | gi\|294710331\|gb\|GW780016.1\|GW780016_PBC019E11_04444_Parthenium_ |
| | argentatum_cold_acclimated_bark_cDNA_library_(PBC)_Parthenium_argentatum_ |
| | cDNA_clone_PBC019E11,_mRNA_sequence |
| 30 | gi\|294710302\|gb\|GW779987.1\|GW779987_PBC019C04_04415_Parthenium_ |
| | argentatum_cold_acclimated_bark_cDNA_library_(PBC)_Parthenium_argentatum_ |
| | cDNA_clone_PBC019C04,_mRNA_sequence |
| 31 | gi\|294710293\|gb\|GW779978.1\|GW779987_PBC019B06_04406_Parthenium_ |
| | argentatum_cold_acclimated_bark_cDNA_library_(PBC)_Parthenium_argentatum_ |
| | cDNA_clone_PBC019B06,_mRNA_sequence |
| 32 | gi\|294710290\|gb\|GW779975.1\|GW779975_PBC019B03_04403_Parthenium_ |
| | argentatum_cold_acclimated_bark_cDNA_library_(PBC)_Parthenium_argentatum_ |
| | cDNA_clone_PBC019B03,_mRNA_sequence |
| 33 | gi\|294710029\|gb\|GW779956.1\|GW779956_PBC018H08_04384_Parthenium_ |
| | argentatum_cold_acclimated_bark_cDNA_library_(PBC)_Parthenium_argentatum_ |
| | cDNA_clone_PBC018H08,_mRNA_sequence |
| 34 | gi\|294710004\|gb\|GW779931.1\|GW779931_PBC018F06_04359_Parthenium_ |
| | argentatum_cold_acclimated_bark_cDNA_library_(PBC)_Parthenium_argentatum_ |
| | cDNA_clone_PBC018F06,_mRNA_sequence |
| 35 | gi\|29470997\|gb\|GW779903.1\|GW779903_PBC018D02_04331_Parthenium_ |
| | argentatum_cold_acclimated_bark_cDNA_library_(PBC)_Parthenium_argentatum_ |
| | cDNA_clone_PBC018D02,_mRNA_sequence |
| 36 | gi\|294709860\|gb\|GW777341.1\|GW777341_PAB921A03_01769_Parthenium_ |
| | argentatum_cold_acclimated_bark_cDNA_library_(PAB)_Parthenium_argentatum_ |
| | cDNA_clone_PAB921A03,_mRNA_sequence |
| 37 | gi\|294709847\|gb\|GW777328.1\|GW777328_PAB921E06_01756_Parthenium_ |
| | argentatum_cold_acclimated_bark_cDNA_library_(PAB)_Parthenium_argentatum_ |
| | cDNA_clone_PAB921A03,_mRNA_sequence |
| 38 | gi\|294709845\|gb\|GW777326.1\|GW777326_PAB921H08_01754_Parthenium_ |
| | argentatum_cold_acclimated_bark_cDNA_library_(PAB)_Parthenium_argentatum_ |
| | cDNA_clone_PAB921H08,_mRNA_sequence |
| 39 | gi\|294709828\|gb\|GW779309.1\|GW777309_PAB921H01_01737_Parthenium_ |
| | argentatum_cold_acclimated_bark_cDNA_library_(PAB)_Parthenium_argentatum_ |
| | cDNA_clone_PAB921H01,_mRNA_sequence |
| 40 | gi\|294709779\|gb\|GW777260.1\|GW777260_PAB921B01_01688_Parthenium_ |
| | argentatum_cold_acclimated_bark_cDNA_library_(PAB)_Parthenium_argentatum_ |
| | cDNA_clone_PAB921B01,_mRNA_sequence |
| 41 | gi\|294709686\|gb\|GW777175.1\|GW777175_PAB917E10_01603_Parthenium_ |
| | argentatum_cold_acclimated_bark_cDNA_library_(PAB)_Parthenium_argentatum_ |
| | cDNA_clone_PAB917E10,_mRNA_sequence |
| 42 | gi\|294709672\|gb\|GW777161.1\|GW777161_PAB920C05_01589_Parthenium_ |
| | argentatum_cold_acclimated_bark_cDNA_library_(PAB)_Parthenium_argentatum_ |
| | cDNA_clone_PAB920C05,_mRNA_sequence |
| 43 | gi\|294709660\|gb\|GW777149.1\|GW777149_PAB920A04_01577_Parthenium_ |
| | argentatum_cold_acclimated_bark_cDNA_library_(PAB)_Parthenium_argentatum_ |
| | cDNA_clone_PAB920A04,_mRNA_sequence |
| 44 | gi\|294709646\|gb\|GW777132.1\|GW777135_PAB920E07_01563_Parthenium_ |
| | argentatum_cold_acclimated_bark_cDNA_library_(PAB)_Parthenium_argentatum_ |
| | cDNA_clone_PAB920E07,_mRNA_sequence |
| 45 | gi\|294709578\|gb\|GW777067.1\|GW777067_PAB916D04_01495_Parthenium_ |
| | argentatum_cold_acclimated_bark_cDNA_library_(PAB)_Parthenium_argentatum_ |
| | cDNA_clone_PAB916D04,_mRNA_sequence |
| 46 | gi\|294709568\|gb\|GW777057.1\|GW777057_PAB916F08_01485_Parthenium_ |
| | argentatum_cold_acclimated_bark_cDNA_library_(PAB)_Parthenium_argentatum_ |
| | cDNA_clone_PAB916F08,_mRNA_sequence |
| 47 | gi\|294709553\|gb\|GW777042.1\|GW777042_PAB916F10_01470_Parthenium_ |
| | argentatum_cold_acclimated_bark_cDNA_library_(PAB)_Parthenium_argentatum_ |
| | cDNA_clone_PAB916F10,_mRNA_sequence |
| 48 | gi\|29470954\|gb\|GW777030.1\|GW777030_PAB916A12_01458_Parthenium_ |
| | argentatum_cold_acclimated_bark_cDNA_library_(PAB)_Parthenium_argentatum_ |
| | cDNA_clone_PAB916A12,_mRNA_sequence |
| 49 | gi\|294709524\|gb\|GW777013.1\|GW777013_PAB916D03_01441_Parthenium_ |
| | argentatum_cold_acclimated_bark_cDNA_library_(PAB)_Parthenium_argentatum_ |
| | cDNA_clone_PAB916D03,_mRNA_sequence |
| 50 | gi\|294709520\|gb\|GW777009.1\|GW777009_PAB916D12_01437_Parthenium_ |
| | argentatum_cold_acclimated_bark_cDNA_library_(PAB)_Parthenium_argentatum_ |
| | cDNA_clone_PAB916D12,_mRNA_sequence |
| 51 | gi\|294709515\|gb\|GW777004.1\|GW777004_PAB916B07_01432_Parthenium_ |
| | argentatum_cold_acclimated_bark_cDNA_library_(PAB)_Parthenium_argentatum_ |
| | cDNA_clone_PAB916B07,_mRNA_sequence |
| 52 | gi\|294709515\|gb\|GW777004.1\|GW777004_PAB916B07_01432_Parthenium_ |
| | argentatum_cold_acclimated_bark_cDNA_library_(PAB)_Parthenium_argentatum_ |
| | cDNA_clone_PAB916B07,_mRNA_sequence |
| 53 | gi\|294709510\|gb\|GW776999.1\|GW776999_PAB916H06_01427_Parthenium_ |
| | argentatum_cold_acclimated_bark_cDNA_library_(PAB)_Parthenium_argentatum_ |
| | cDNA_clone_PAB916H06,_mRNA_sequence |
| 54 | gi\|294709468\|gb\|GW776957.1\|GW776957_PAB915H08_01385_Parthenium_ |
| | argentatum_cold_acclimated_bark_cDNA_library_(PAB)_Parthenium_argentatum_ |
| | cDNA_clone_PAB915H08,_mRNA_sequence |
| 55 | gi\|294709448\|gb\|GW776937.1\|GW776937_PAB915D01_01365_Parthenium_ |
| | argentatum_cold_acclimated_bark_cDNA_library_(PAB)_Parthenium_argentatum_ |
| | cDNA_clone_PAB915D01,_mRNA_sequence |
| 56 | gi\|294709422\|gb\|GW776911.1\|GW776911_PAB915C10_01339_Parthenium_ |
| | argentatum_cold_acclimated_bark_cDNA_library_(PAB)_Parthenium_argentatum_ |
| | cDNA_clone_PAB915C10,_mRNA_sequence |
| 57 | gi\|294709394\|gb\|GW776883.1\|GW776883_PAB919E03_01311_Parthenium_ |
| | argentatum_cold_acclimated_bark_cDNA_library_(PAB)_Parthenium_argentatum_ |
| | cDNA_clone_PAB919E03,_mRNA_sequence |
| 58 | gi\|294709386\|gb\|GW776875.1\|GW776875_PAB919G02_01303_Parthenium_ |
| | argentatum_cold_acclimated_bark_cDNA_library_(PAB)_Parthenium_argentatum_ |
| | cDNA_clone_PAB919G02,_mRNA_sequence |
| 59 | gi\|294709361\|gb\|GW776850.1\|GW776850_PAB919F02_01278_Parthenium_ |
| | argentatum_cold_acclimated_bark_cDNA_library_(PAB)_Parthenium_argentatum_ |
| | cDNA_clone_PAB919F02,_mRNA_sequence |
| 60 | gi\|294709355\|gb\|GW776844.1\|GW776844_PAB919H01_01272_Parthenium_ |
| | argentatum_cold_acclimated_bark_cDNA_library_(PAB)_Parthenium_argentatum_ |
| | cDNA_clone_PAB919H01,_mRNA_sequence |
| 61 | gi\|294709355\|gb\|GW776844.1\|GW776844_PAB919H01_01272_Parthenium_ |
| | argentatum_cold_acclimated_bark_cDNA_library_(PAB)_Parthenium_argentatum_ |
| | cDNA_clone_PAB919H01,_mRNA_sequence |
| 62 | gi\|294709292\|gb\|GW776781.1\|GW776781_PAB918F07_01209_Parthenium_ |
| | argentatum_cold_acclimated_bark_cDNA_library_(PAB)_Parthenium_argentatum_ |
| | cDNA_clone_PAB918F07,_mRNA_sequence |
| 63 | gi\|294709291\|gb\|GW776780.1\|GW776780_PAB915F11_01208_Parthenium_ |
| | argentatum_cold_acclimated_bark_cDNA_library_(PAB)_Parthenium_argentatum_ |
| | cDNA_clone_PAB918F11,_mRNA_sequence |
| 64 | gi\|294709274\|gb\|GW776763.1\|GW776763_PAB918C08_01191_Parthenium_ |
| | argentatum_cold_acclimated_bark_cDNA_library_(PAB)_Parthenium_argentatum_ |
| | cDNA_clone_PAB918C08,_mRNA_sequence |
| 65 | gi\|294709113\|gb\|GW783004.1\|GW783004_PBC052F11_07432_Parthenium_ |
| | argentatum_cold_acclimated_bark_cDNA_library_(PBC)_Parthenium_argentatum_ |
| | cDNA_clone_PBC052F11,_mRNA_sequence |
| 66 | gi\|294709108\|gb\|GW782999.1\|GW782999_PBC052F06_07427_Parthenium_ |
| | argentatum_cold_acclimated_bark_cDNA_library_(PBC)_Parthenium_argentatum_ |
| | cDNA_clone_PBC052F06,_mRNA_sequence |
| 67 | gi\|294709094\|gb\|GW782985.1\|GW782985_PBC052E05_07413_Parthenium_ |
| | argentatum_cold_acclimated_bark_cDNA_library_(PBC)_Parthenium_argentatum_ |
| | cDNA_clone_PBC052E05,_mRNA_sequence |
| 68 | gi\|294709089\|gb\|GW782980.1\|GW782980_PBC052D10_07408_Parthenium_ |
| | argentatum_cold_acclimated_bark_cDNA_library_(PBC)_Parthenium_argentatum_ |
| | cDNA_clone_PBC052D10,_mRNA_sequence |
| 69 | gi\|294708962\|gb\|GW782935.1\|GW782935_PBC051H08_07363_Parthenium_ |
| | argentatum_cold_acclimated_bark_cDNA_library_(PBC)_Parthenium_argentatum_ |
| | cDNA_clone_PBC051H08,_mRNA_sequence |
| 70 | gi\|294708913\|gb\|GW782886.1\|GW782886_PBC051D05_07314_Parthenium_ |
| | argentatum_cold_acclimated_bark_cDNA_library_(PBC)_Parthenium_argentatum_ |
| | cDNA_clone_PBC051D05,_mRNA_sequence |
| 71 | gi\|294708913\|gb\|GW782886.1\|GW782886_PBC051D05_07314_Parthenium_ |
| | argentatum_cold_acclimated_bark_cDNA_library_(PBC)_Parthenium_argentatum_ |
| | cDNA_clone_PBC051D05,_mRNA_sequence |
| 72 | gi\|294708812\|gb\|GW782867.1\|GW782867_PBC051B06_07295_Parthenium_ |
| | argentatum_cold_acclimated_bark_cDNA_library_(PBC)_Parthenium_argentatum_ |
| | cDNA_clone_PBC051B06,_mRNA_sequence |
| 73 | gi\|294708802\|gb\|GW782857.1\|GW782857_PBC051A08_07285_Parthenium_ |
| | argentatum_cold_acclimated_bark_cDNA_library_(PBC)_Parthenium_argentatum_ |
| | cDNA_clone_PBC051A08,_mRNA_sequence |
| 74 | gi\|294708796\|gb\|GW782851.1\|GW782851_PBC051A05_07279_Parthenium_ |
| | argentatum_cold_acclimated_bark_cDNA_library_(PBC)_Parthenium_argentatum_ |
| | cDNA_clone_PBC051A05,_mRNA_sequence |
| 75 | gi\|294708654\|gb\|GW782279.1\|GW782791_PBC050C11_07219_Parthenium_ |
| | argentatum_cold_acclimated_bark_cDNA_library_(PBC)_Parthenium_argentatum_ |
| | cDNA_clone_PBC050C11,_mRNA_sequence |
| 76 | gi\|294708635\|gb\|GW782772.1\|GW782772_PBC050B05_07200_Parthenium_ |
| | argentatum_cold_acclimated_bark_cDNA_library_(PBC)_Parthenium_argentatum_ |
| | cDNA_clone_PBC050B05,_mRNA_sequence |
| 77 | gi\|294708630\|gb\|GW782767.1\|GW782767_PBC050A11_07195_Parthenium_ |
| | argentatum_cold_acclimated_bark_cDNA_library_(PBC)_Parthenium_argentatum_ |
| | cDNA_clone_PBC050A11,_mRNA_sequence |
| 78 | gi\|294708611\|gb\|GW782748.1\|GW782748_PBC049H04_07176_Parthenium_ |
| | argentatum_cold_acclimated_bark_cDNA_library_(PBC)_Parthenium_argentatum_ |
| | cDNA_clone_PBC049H04,_mRNA_sequence |
| 79 | gi\|294708499\|gb\|GW782718.1\|GW782718_PBC049E06_07146_Parthenium_ |
| | argentatum_cold_acclimated_bark_cDNA_library_(PBC)_Parthenium_argentatum_ |
| | cDNA_clone_PBC049E06,_mRNA_sequence |
| 80 | gi\|294708474\|gb\|GW782693.1\|GW782693_PBC049C05_07121_Parthenium_ |
| | argentatum_cold_acclimated_bark_cDNA_library_(PBC)_Parthenium_argentatum_ |
| | cDNA_clone_PBC049C05,_mRNA_sequence |
| 81 | gi\|294708471\|gb\|GW782690.1\|GW782690_PBC049C04_07118_Parthenium_ |
| | argentatum_cold_acclimated_bark_cDNA_library_(PBC)_Parthenium_argentatum_ |
| | cDNA_clone_PBC049C04,_mRNA_sequence |
| 82 | gi\|294708461\|gb\|GW782680.1\|GW782680_PBC049B04_07108_Parthenium_ |
| | argentatum_cold_acclimated_bark_cDNA_library_(PBC)_Parthenium_argentatum_ |
| | cDNA_clone_PBC049B04,_mRNA_sequence |
| 83 | gi\|294708450\|gb\|GW782669.1\|GW782669_PBC049A04_07097_Parthenium_ |
| | argentatum_cold_acclimated_bark_cDNA_library_(PBC)_Parthenium_argentatum_ |
| | cDNA_clone_PBC049A04,_mRNA_sequence |
| 84 | gi\|294708436\|gb\|GW782655.1\|GW782655_PBC048G09_07083_Parthenium_ |
| | argentatum_cold_acclimated_bark_cDNA_library_(PBC)_Parthenium_argentatum_ |
| | cDNA_clone_PBC048G09,_mRNA_sequence |
| 85 | gi\|294708167\|gb\|GW782550.1\|GW782550_PBC047E11_06978_Parthenium_ |
| | argentatum_cold_acclimated_bark_cDNA_library_(PBC)_Parthenium_argentatum_ |
| | cDNA_clone_PBC047E11,_mRNA_sequence |
| 86 | gi\|294708158\|gb\|GW782541.1\|GW782541_PBC047E04_06969_Parthenium_ |
| | argentatum_cold_acclimated_bark_cDNA_library_(PBC)_Parthenium_argentatum_ |
| | cDNA_clone_PBC047E04,_mRNA_sequence |
| 87 | gi\|294708131\|gb\|GW782514.1\|GW782514_PBC047B08_06942_Parthenium_ |
| | argentatum_cold_acclimated_bark_cDNA_library_(PBC)_Parthenium_argentatum_ |
| | cDNA_clone_PBC047B08,_mRNA_sequence |
| 88 | gi\|294707986\|gb\|GW782451.1\|GW782451_PBC047E04_06879_Parthenium_ |
| | argentatum_cold_acclimated_bark_cDNA_library_(PBC)_Parthenium_argentatum_ |
| | cDNA_clone_PBC046E04,_mRNA_sequence |
| 89 | gi\|294707981\|gb\|GW782446.1\|GW782446_PBC046D08_06874_Parthenium_ |
| | argentatum_cold_acclimated_bark_cDNA_library_(PBC)_Parthenium_argentatum_ |
| | cDNA_clone_PBC046D08,_mRNA_sequence |
| 90 | gi\|294707957\|gb\|GW782422.1\|GW782422_PBC047B06_06850_Parthenium_ |
| | argentatum_cold_acclimated_bark_cDNA_library_(PBC)_Parthenium_argentatum_ |
| | cDNA_clone_PBC046B06,_mRNA_sequence |
| 91 | gi\|294707950\|gb\|GW782415.1\|GW782415_PBC046A11_06843_Parthenium_ |
| | argentatum_cold_acclimated_bark_cDNA_library_(PBC)_Parthenium_argentatum_ |
| | cDNA_clone_PBC046A11,_mRNA_sequence |
| 92 | gi\|294705882\|gb\|GW779867.1\|GW779867_PBC017H12_04295_Parthenium_ |
| | argentatum_cold_acclimated_bark_cDNA_library_(PBC)_Parthenium_argentatum_ |
| | cDNA_clone_PBC017H12,_mRNA_sequence |
| 93 | gi\|294705836\|gb\|GW779821.1\|GW779821_PBC017E01_04249_Parthenium_ |
| | argentatum_cold_acclimated_bark_cDNA_library_(PBC)_Parthenium_argentatum_ |
| | cDNA_clone_PBC017E01,_mRNA_sequence |
| 94 | gi\|294705713\|gb\|GW779778.1\|GW779778_PBC017A05_04206_Parthenium_ |
| | argentatum_cold_acclimated_bark_cDNA_library_(PBC)_Parthenium_argentatum_ |
| | cDNA_clone_PBC017A05,_mRNA_sequence |
| 95 | gi\|294705712\|gb\|GW779777.1\|GW779777_PBC017A03_04205_Parthenium_ |
| | argentatum_cold_acclimated_bark_cDNA_library_(PBC)_Parthenium_argentatum_ |
| | cDNA_clone_PBC017A03,_mRNA_sequence |
| 96 | gi\|294705671\|gb\|GW779736.1\|GW779736_PBC016E09_04164_Parthenium_ |
| | argentatum_cold_acclimated_bark_cDNA_library_(PBC)_Parthenium_argentatum_ |
| | cDNA_clone_PBC016E09,_mRNA_sequence |
| 97 | gi\|294705562\|gb\|GW779707.1\|GW779707_PBC016C02_04135_Parthenium_ |
| | argentatum_cold_acclimated_bark_cDNA_library_(PBC)_Parthenium_argentatum_ |
| | cDNA_clone_PBC016C02,_mRNA_sequence |
| 98 | gi\|294705547\|gb\|GW779692.1\|GW779692_PBC016A11_04120_Parthenium_ |
| | argentatum_cold_acclimated_bark_cDNA_library_(PBC)_Parthenium_argentatum_ |
| | cDNA_clone_PBC016A11,_mRNA_sequence |
| 99 | gi\|294705520\|gb\|GW779665.1\|GW779665_PBC015G05_04093_Parthenium_ |
| | argentatum_cold_acclimated_bark_cDNA_library_(PBC)_Parthenium_argentatum_ |
| | cDNA_clone_PBC015G05,_mRNA_sequence |
| 100 | gi\|294705513\|gb\|GW779658.1\|GW779658_PBC015F10_04086_Parthenium_ |
| | argentatum_cold_acclimated_bark_cDNA_library_(PBC)_Parthenium_argentatum_ |
| | cDNA_clone_PBC015F10,_mRNA_sequence |
| 101 | gi\|294705497\|gb\|GW779642.1\|GW779642_PBC015E06_04070_Parthenium_ |
| | argentatum_cold_acclimated_bark_cDNA_library_(PBC)_Parthenium_argentatum_ |
| | cDNA_clone_PBC015E06,_mRNA_sequence |
| 102 | gi\|294705339\|gb\|GW779634.1\|GW779634_PBC015D10_04062_Parthenium_ |
| | argentatum_cold_acclimated_bark_cDNA_library_(PBC)_Parthenium_argentatum_ |
| | cDNA_clone_PBC015D10,_mRNA_sequence |
| 103 | gi\|294705334\|gb\|GW779629.1\|GW779629_PBC015D05_04057_Parthenium_ |
| | argentatum_cold_acclimated_bark_cDNA_library_(PBC)_Parthenium_argentatum_ |
| | cDNA_clone_PBC015D05,_mRNA_sequence |
| 104 | gi\|294705323\|gb\|GW779618.1\|GW779618_PBC015C06_04046_Parthenium_ |
| | argentatum_cold_acclimated_bark_cDNA_library_(PBC)_Parthenium_argentatum_ |
| | cDNA_clone_PBC015C06,_mRNA_sequence |
| 105 | gi\|294705284\|gb\|GW779579.1\|GW779579_PBC014H03_04007_Parthenium_ |
| | argentatum_cold_acclimated_bark_cDNA_library_(PBC)_Parthenium_argentatum_ |
| | cDNA_clone_PBC014H03,_mRNA_sequence |
| 106 | gi\|294705282\|gb\|GW779577.1\|GW779577_PBC014H01_04005_Parthenium_ |
| | argentatum_cold_acclimated_bark_cDNA_library_(PBC)_Parthenium_argentatum_ |
| | cDNA_clone_PBC014H01,_mRNA_sequence |
| 107 | gi\|294705281\|gb\|GW779576.1\|GW779576_PBC014G12_04004_Parthenium_ |
| | argentatum_cold_acclimated_bark_cDNA_library_(PBC)_Parthenium_argentatum_ |
| | cDNA_clone_PBC014G12,_mRNA_sequence |
| 108 | gi\|294705090\|gb\|GW779545.1\|GW779545_PBC014E03_03973_Parthenium_ |
| | argentatum_cold_acclimated_bark_cDNA_library_(PBC)_Parthenium_argentatum_ |
| | cDNA_clone_PBC014E03,_mRNA_sequence |
| 109 | gi\|294705064\|gb\|GW779519.1\|GW779519_PBC014B11_03947_Parthenium_ |
| | argentatum_cold_acclimated_bark_cDNA_library_(PBC)_Parthenium_argentatum_ |
| | cDNA_clone_PBC014B11,_mRNA_sequence |
| 110 | gi\|294705052\|gb\|GW779507.1\|GW779507_PBC014A11_03935_Parthenium_ |
| | argentatum_cold_acclimated_bark_cDNA_library_(PBC)_Parthenium_argentatum_ |
| | cDNA_clone_PBC014B11,_mRNA_sequence |
| 111 | gi\|294705050\|gb\|GW779505.1\|GW779505_PBC014A09_03933_Parthenium_ |
| | argentatum_cold_acclimated_bark_cDNA_library_(PBC)_Parthenium_argentatum_ |
| | cDNA_clone_PBC014A09,_mRNA_sequence |
| 112 | gi\|294705032\|gb\|GW779487.1\|GW779487_PBC013H03_03915_Parthenium_ |
| | argentatum_cold_acclimated_bark_cDNA_library_(PBC)_Parthenium_argentatum_ |
| | cDNA_clone_PBC013H03,_mRNA_sequence |
| 113 | gi\|294704868\|gb\|GW779483.1\|GW779483_PBC013G10_03911_Parthenium_ |
| | argentatum_cold_acclimated_bark_cDNA_library_(PBC)_Parthenium_argentatum_ |
| | cDNA_clone_PBC013G10,_mRNA_sequence |
| 114 | gi\|294704861\|gb\|GW779476.1\|GW779476_PBC013G02_03904_Parthenium_ |
| | argentatum_cold_acclimated_bark_cDNA_library_(PBC)_Parthenium_argentatum_ |
| | cDNA_clone_PBC013G02,_mRNA_sequence |
| 115 | gi\|294704854\|gb\|GW779469.1\|GW779469_PBC013F07_03897_Parthenium_ |
| | argentatum_cold_acclimated_bark_cDNA_library_(PBC)_Parthenium_argentatum_ |
| | cDNA_clone_PBC013F07,_mRNA_sequence |
| 116 | gi\|294704603\|gb\|GW779378.1\|GW779378_PBC012F09_03806_Parthenium_ |
| | argentatum_cold_acclimated_bark_cDNA_library_(PBC)_Parthenium_argentatum_ |
| | cDNA_clone_PBC012F09,_mRNA_sequence |
| 117 | gi\|294704581\|gb\|GW779356.1\|GW779356_PBC012D10_03784_Parthenium_ |
| | argentatum_cold_acclimated_bark_cDNA_library_(PBC)_Parthenium_argentatum_ |
| | cDNA_clone_PBC012D10,_mRNA_sequence |
| 118 | gi\|294704578\|gb\|GW779353.1\|GW779353_PBC012D07_03781_Parthenium_ |
| | argentatum_cold_acclimated_bark_cDNA_library_(PBC)_Parthenium_argentatum_ |
| | cDNA_clone_PBC012D07,_mRNA_sequence |
| 119 | gi\|294704434\|gb\|GW779289.1\|GW779289_PBC011F10_03717_Parthenium_ |
| | argentatum_cold_acclimated_bark_cDNA_library_(PBC)_Parthenium_argentatum_ |
| | cDNA_clone_PBC011F10,_mRNA_sequence |
| 120 | gi\|294704432\|gb\|GW779287.1\|GW779287_PBC011F08_03715_Parthenium_ |
| | argentatum_cold_acclimated_bark_cDNA_library_(PBC)_Parthenium_argentatum_ |
| | cDNA_clone_PBC011F08,_mRNA_sequence |
| 121 | gi\|294704426\|gb\|GW779281.1\|GW779281_PBC011E12_03709_Parthenium_ |
| | argentatum_cold_acclimated_bark_cDNA_library_(PBC)_Parthenium_argentatum_ |
| | cDNA_clone_PBC011E12,_mRNA_sequence |
| 122 | gi\|294704425\|gb\|GW779280.1\|GW779280_PBC011E11_03708_Parthenium_ |
| | argentatum_cold_acclimated_bark_cDNA_library_(PBC)_Parthenium_argentatum_ |
| | cDNA_clone_PBC011E11,_mRNA_sequence |
| 123 | gi\|294704407\|gb\|GW779262.1\|GW779262_PBC011D04_03690_Parthenium_ |
| | argentatum_cold_acclimated_bark_cDNA_library_(PBC)_Parthenium_argentatum_ |
| | cDNA_clone_PBC011D04,_mRNA_sequence |
| 124 | gi\|294704403\|gb\|GW779258.1\|GW779258_PBC011C12_03686_Parthenium_ |
| | argentatum_cold_acclimated_bark_cDNA_library_(PBC)_Parthenium_argentatum_ |
| | cDNA_clone_PBC011C12,_mRNA_sequence |
| 125 | gi\|294704131\|gb\|GW777731.1\|GW777731_PAB926F11_02159_Parthenium_ |
| | argentatum_cold_acclimated_bark_cDNA_library_(PAB)_Parthenium_argentatum_ |
| | cDNA_clone_PAB926F11,_mRNA_sequence |
| 126 | gi\|294704126\|gb\|GW777726.1\|GW777726_PAB926B11_02154_Parthenium_ |
| | argentatum_cold_acclimated_bark_cDNA_library_(PAB)_Parthenium_argentatum_ |
| | cDNA_clone_PAB926B11,_mRNA_sequence |
| 127 | gi\|294702510\|gb\|GW775984.1\|GW775984_PAB905g09_00412_Parthenium_ |
| | argentatum_cold_acclimated_bark_cDNA_library_(PAB)_Parthenium_argentatum_ |
| | cDNA_clone_PAB905g09,_mRNA_sequence |
| 128 | gi\|294702508\|gb\|GW775982.1\|GW775982_PAB905c04_00410_Parthenium_ |
| | argentatum_cold_acclimated_bark_cDNA_library_(PAB)_Parthenium_argentatum_ |
| | cDNA_clone_PAB905c04,_mRNA_sequence |
| 129 | gi\|294702506\|gb\|GW775980.1\|GW775980_PAB905c09_00408_Parthenium_ |
| | argentatum_cold_acclimated_bark_cDNA_library_(PAB)_Parthenium_argentatum_ |
| | cDNA_clone_PAB905c09,_mRNA_sequence |
| 130 | gi\|294701675\|gb\|GW785540.1\|GW785540_PBC081B09_09968_Parthenium_ |
| | argentatum_cold_acclimated_bark_cDNA_library_(PBC)_Parthenium_argentatum_ |
| | cDNA_clone_PBC081B09,_mRNA_sequence |
| 131 | gi\|294701661\|gb\|GW785526.1\|GW785526_PBC081A06_09954_Parthenium_ |
| | argentatum_cold_acclimated_bark_cDNA_library_(PBC)_Parthenium_argentatum_ |
| | cDNA_clone_PBC081A06,_mRNA_sequence |
| 132 | gi\|294701631\|gb\|GW785496.1\|GW785496_PBC080F11_09924_Parthenium_ |
| | argentatum_cold_acclimated_bark_cDNA_library_(PBC)_Parthenium_argentatum_ |
| | cDNA_clone_PBC080F11,_mRNA_sequence |
| 133 | gi\|294701611\|gb\|GW785476.1\|GW785476_PBC080E05_09904_Parthenium_ |
| | argentatum_cold_acclimated_bark_cDNA_library_(PBC)_Parthenium_argentatum_ |
| | cDNA_clone_PBC080E05,_mRNA_sequence |
| 134 | gi\|294701562\|gb\|GW785427.1\|GW785427_PBC079H08_09855_Parthenium_ |
| | argentatum_cold_acclimated_bark_cDNA_library_(PBC)_Parthenium_argentatum_ |
| | cDNA_clone_PBC079H08,_mRNA_sequence |
| 135 | gi\|294701536\|gb\|GW785401.1\|GW785401_PBC079F05_09829_Parthenium_ |
| | argentatum_cold_acclimated_bark_cDNA_library_(PBC)_Parthenium_argentatum_ |
| | cDNA_clone_PBC079F05,_mRNA_sequence |
| 136 | gi\|294701532\|gb\|GW785397.1\|GW785397_PBC079E07_09825_Parthenium_ |
| | argentatum_cold_acclimated_bark_cDNA_library_(PBC)_Parthenium_argentatum_ |
| | cDNA_clone_PBC079E07,_mRNA_sequence |
| 137 | gi\|294701516\|gb\|GW785381.1\|GW785381_PBC079C09_09809_Parthenium_ |
| | argentatum_cold_acclimated_bark_cDNA_library_(PBC)_Parthenium_argentatum_ |
| | cDNA_clone_PBC079C09,_mRNA_sequence |
| 138 | gi\|294701502\|gb\|GW785367.1\|GW785367_PBC079B05_09795_Parthenium_ |
| | argentatum_cold_acclimated_bark_cDNA_library_(PBC)_Parthenium_argentatum_ |
| | cDNA_clone_PBC079B05,_mRNA_sequence |
| 139 | gi\|294701454\|gb\|GW785319.1\|GW785319_PBC078E12_09747_Parthenium_ |
| | argentatum_cold_acclimated_bark_cDNA_library_(PBC)_Parthenium_argentatum_ |
| | cDNA_clone_PBC078E12,_mRNA_sequence |
| 140 | gi\|294701439\|gb\|GW785304.1\|GW785304_PBC078D08_09732_Parthenium_ |
| | argentatum_cold_acclimated_bark_cDNA_library_(PBC)_Parthenium_argentatum_ |
| | cDNA_clone_PBC078D08,_mRNA_sequence |
| 141 | gi\|294701407\|gb\|GW785272.1\|GW785272_PBC078A12_09700_Parthenium_ |
| | argentatum_cold_acclimated_bark_cDNA_library_(PBC)_Parthenium_argentatum_ |
| | cDNA_clone_PBC078A12,_mRNA_sequence |
| 142 | gi\|294701404\|gb\|GW785269.1\|GW785269_PBC078A09_09697_Parthenium_ |
| | argentatum_cold_acclimated_bark_cDNA_library_(PBC)_Parthenium_argentatum_ |
| | cDNA_clone_PBC078A09,_mRNA_sequence |
| 143 | gi\|294701402\|gb\|GW785267.1\|GW785267_PBC078A06_09695_Parthenium_ |
| | argentatum_cold_acclimated_bark_cDNA_library_(PBC)_Parthenium_argentatum_ |
| | cDNA_clone_PBC078A06,_mRNA_sequence |
| 144 | gi\|294701398\|gb\|GW785263.1\|GW785263_PBC078A05_09691_Parthenium_ |
| | argentatum_cold_acclimated_bark_cDNA_library_(PBC)_Parthenium_argentatum_ |
| | cDNA_clone_PBC078A05,_mRNA_sequence |
| 145 | gi\|294701367\|gb\|GW785232.1\|GW785232_PBC077F06_09660_Parthenium_ |
| | argentatum_cold_acclimated_bark_cDNA_library_(PBC)_Parthenium_argentatum_ |
| | cDNA_clone_PBC077F06,_mRNA_sequence |
| 146 | gi\|294701340\|gb\|GW785205.1\|GW785205_PBC077D05_09633_Parthenium_ |
| | argentatum_cold_acclimated_bark_cDNA_library_(PBC)_Parthenium_argentatum_ |
| | cDNA_clone_PBC077D05,_mRNA_sequence |
| 147 | gi\|294701308\|gb\|GW785173.1\|GW785173_PBC077A05_09601_Parthenium_ |
| | argentatum_cold_acclimated_bark_cDNA_library_(PBC)_Parthenium_argentatum_ |
| | cDNA_clone_PBC077A05,_mRNA_sequence |
| 148 | gi\|294701291\|gb\|GW785156.1\|GW785156_PBC076G11_09584_Parthenium_ |
| | argentatum_cold_acclimated_bark_cDNA_library_(PBC)_Parthenium_argentatum_ |
| | cDNA_clone_PBC076G11,_mRNA_sequence |
| 149 | gi\|294701289\|gb\|GW785154.1\|GW785154_PBC076G09_09582_Parthenium_ |
| | argentatum_cold_acclimated_bark_cDNA_library_(PBC)_Parthenium_argentatum_ |
| | cDNA_clone_PBC076G09,_mRNA_sequence |
| 150 | gi\|294701279\|gb\|GW785144.1\|GW785144_PBC076F11_09572_Parthenium_ |
| | argentatum_cold_acclimated_bark_cDNA_library_(PBC)_Parthenium_argentatum_ |
| | cDNA_clone_PBC076F11,_mRNA_sequence |
| 151 | gi\|294701279\|gb\|GW785144.1\|GW785144_PBC076F11_09572_Parthenium_ |
| | argentatum_cold_acclimated_bark_cDNA_library_(PBC)_Parthenium_argentatum_ |
| | cDNA_clone_PBC076F11,_mRNA_sequence |
| 152 | gi\|294701276\|gb\|GW785141.1\|GW785141_PBC076F08_09569_Parthenium_ |
| | argentatum_cold_acclimated_bark_cDNA_library_(PBC)_Parthenium_argentatum_ |
| | cDNA_clone_PBC076F08,_mRNA_sequence |
| 153 | gi\|294701260\|gb\|GW785125.1\|GW785125_PBC076E05_09553_Parthenium_ |
| | argentatum_cold_acclimated_bark_cDNA_library_(PBC)_Parthenium_argentatum_ |
| | cDNA_clone_PBC076E05,_mRNA_sequence |
| 154 | gi\|294701245\|gb\|GW785110.1\|GW785110_PBC076C11_09538_Parthenium_ |
| | argentatum_cold_acclimated_bark_cDNA_library_(PBC)_Parthenium_argentatum_ |
| | cDNA_clone_PBC076C11,_mRNA_sequence |
| 155 | gi\|294701171\|gb\|GW785036.1\|GW785036_PBC075E07_09464_Parthenium_ |
| | argentatum_cold_acclimated_bark_cDNA_library_(PBC)_Parthenium_argentatum_ |
| | cDNA_clone_PBC075E07,_mRNA_sequence |
| 156 | gi\|294701098\|gb\|GW784963.1\|GW784963_PBC074F10_09391_Parthenium_ |
| | argentatum_cold_acclimated_bark_cDNA_library_(PBC)_Parthenium_argentatum_ |
| | cDNA_clone_PBC074F10,_mRNA_sequence |
| 157 | gi\|294701078\|gb\|GW784943.1\|GW784963_PBC074D11_09371_Parthenium_ |
| | argentatum_cold_acclimated_bark_cDNA_library_(PBC)_Parthenium_argentatum_ |
| | cDNA_clone_PBC074D11,_mRNA_sequence |
| 158 | gi\|294699459\|gb\|GW782279.1\|GW782279_PBC044F04_06707_Parthenium_ |
| | argentatum_cold_acclimated_bark_cDNA_library_(PBC)_Parthenium_argentatum_ |
| | cDNA_clone_PBC044F04,_mRNA_sequence |
| 159 | gi\|294699449\|gb\|GW782269.1\|GW782269_PBC044E04_06697_Parthenium_ |
| | argentatum_cold_acclimated_bark_cDNA_library_(PBC)_Parthenium_argentatum_ |
| | cDNA_clone_PBC044E04,_mRNA_sequence |
| 160 | gi\|294699424\|gb\|GW776635.1\|GW776635_PAB913D06_01063_Parthenium_ |
| | argentatum_cold_acclimated_bark_cDNA_library_(PAB)_Parthenium_argentatum_ |
| | cDNA_clone_PAB913D06,_mRNA_sequence |
| 161 | gi\|294699406\|gb\|GW776617.1\|GW776617_PAB913B03_01045_Parthenium_ |
| | argentatum_cold_acclimated_bark_cDNA_library_(PAB)_Parthenium_argentatum_ |
| | cDNA_clone_PAB913B03,_mRNA_sequence |
| 162 | gi\|294699252\|gb\|GW782233.1\|GW782233_PBC044A11_06661_Parthenium_ |
| | argentatum_cold_acclimated_bark_cDNA_library_(PBC)_Parthenium_argentatum_ |
| | cDNA_clone_PBC044A11,_mRNA_sequence |
| 163 | gi\|294699231\|gb\|GW782212.1\|GW782212_PBC043G10_06640_Parthenium_ |
| | argentatum_cold_acclimated_bark_cDNA_library_(PBC)_Parthenium_argentatum_ |
| | cDNA_clone_PBC043G10,_mRNA_sequence |
| 164 | gi\|294699224\|gb\|GW782205.1\|GW782205_PBC043G04_06633_Parthenium_ |
| | argentatum_cold_acclimated_bark_cDNA_library_(PBC)_Parthenium_argentatum_ |
| | cDNA_clone_PBC043G04,_mRNA_sequence |
| 165 | gi\|294699198\|gb\|GW782179.1\|GW782179_PBC043E04_06607_Parthenium_ |
| | argentatum_cold_acclimated_bark_cDNA_library_(PBC)_Parthenium_argentatum_ |
| | cDNA_clone_PBC043E04,_mRNA_sequence |
| 166 | gi\|294699189\|gb\|GW782170.1\|GW78217_PBC043D04_06598_Parthenium_ |
| | argentatum_cold_acclimated_bark_cDNA_library_(PBC)_Parthenium_argentatum_ |
| | cDNA_clone_PBC043D04,_mRNA_sequence |
| 167 | gi\|294699188\|gb\|GW776560.1\|GW776560_PAB912d07_00988_Parthenium_ |
| | argentatum_cold_acclimated_bark_cDNA_library_(PAB)_Parthenium_argentatum_ |
| | cDNA_clone_PAB912d07,_mRNA_sequence |
| 168 | gi\|294699173\|gb\|GW776545.1\|GW776545_PAB912a12_00973_Parthenium_ |
| | argentatum_cold_acclimated_bark_cDNA_library_(PAB)_Parthenium_argentatum_ |
| | cDNA_clone_PAB912a12,_mRNA_sequence |
| 169 | gi\|294699152\|gb\|GW776524.1\|GW776524_PAB912f07_00952_Parthenium_ |
| | argentatum_cold_acclimated_bark_cDNA_library_(PAB)_Parthenium_argentatum_ |
| | cDNA_clone_PAB912f07,_mRNA_sequence |
| 170 | gi\|294699137\|gb\|GW776509.1\|GW776509_PAB912d10_00937_Parthenium_ |
| | argentatum_cold_acclimated_bark_cDNA_library_(PBC)_Parthenium_argentatum_ |
| | cDNA_clone_PAB912d10,_mRNA_sequence |
| 171 | gi\|294699026\|gb\|GW782168.1\|GW784963_PBC043D04_06596_Parthenium_ |
| | argentatum_cold_acclimated_bark_cDNA_library_(PBC)_Parthenium_argentatum_ |
| | cDNA_clone_PBC043D04,_mRNA_sequence |
| 172 | gi\|294698976\|gb\|GW782118.1\|GW782118_PBC042G05_06546_Parthenium_ |
| | argentatum_cold_acclimated_bark_cDNA_library_(PBC)_Parthenium_argentatum_ |
| | cDNA_clone_PBC042G05,_mRNA_sequence |
| 173 | gi\|294698964\|gb\|GW782106.1\|GW782106_PBC042F05_06534_Parthenium_ |
| | argentatum_cold_acclimated_bark_cDNA_library_(PBC)_Parthenium_argentatum_ |
| | cDNA_clone_PBC042F05,_mRNA_sequence |
| 174 | gi\|294698952\|gb\|GW782094.1\|GW782094_PBC042E04_06522_Parthenium_ |
| | argentatum_cold_acclimated_bark_cDNA_library_(PBC)_Parthenium_argentatum_ |
| | cDNA_clone_PBC042E04,_mRAN_sequence |
| 175 | gi\|294698943\|gb\|GW776476.1\|GW776476_PAB911c07_00904_Parthenium_ |
| | argentatum_cold_acclimated_bark_cDNA_library_(PAB)_Parthenium_argentatum_ |
| | cDNA_clone_PAB911c07,_mRAN_sequence |
| 176 | gi\|294698940\|gb\|GW776473.1\|GW776473_PAB911f09_00901_Parthenium_ |
| | argentatum_cold_acclimated_bark_cDNA_library_(PAB)_Parthenium_argentatum_ |
| | cDNA_clone_PAB911f09,_mRNA_sequence |
| 177 | gi\|294698908\|gb\|GW776441.1\|GW776441_PAB911c03_00869_Parthenium_ |
| | argentatum_cold_acclimated_bark_cDNA_library_(PAB)_Parthenium_argentatum_ |
| | cDNA_clone_PAB911c03,_mRNA_sequence |
| 178 | gi\|294698892\|gb\|GW776425.1\|GW776425_PAB911c01_00853_Parthenium_ |
| | argentatum_cold_acclimated_bark_cDNA_library_(PAB)_Parthenium_argentatum_ |
| | cDNA_clone_PAB911c01,_mRNA_sequence |
| 179 | gi\|294698874\|gb\|GW776407.1\|GW776407_PAB911f01_00835_Parthenium_ |
| | argentatum_cold_acclimated_bark_cDNA_library_(PAB)_Parthenium_argentatum_ |
| | cDNA_clone_PAB911f01,_mRNA_sequence |
| 180 | gi\|294698726\|gb\|GW782029.1\|GW782029_PBC041G06_06457_Parthenium_ |
| | argentatum_cold_acclimated_bark_cDNA_library_(PBC)_Parthenium_argentatum_ |
| | cDNA_clone_PBC041G06,_mRNA_sequence |
| 181 | gi\|294698540\|gb\|GW782004.1\|GW782004_PBC041E04_06432_Parthenium_ |
| | argentatum_cold_acclimated_bark_cDNA_library_(PBC)_Parthenium_argentatum_ |
| | cDNA_clone_PBC041E04,_mRNA_sequence |
| 182 | gi\|294698539\|gb\|GW782003.1_GW782003_PBC041E04_06431_Parthenium_ |
| | argentatum_cold_acclimated_bark_cDNA_library_(PBC)_Parthenium_argentatum_ |
| | cDNA_clone_PBC041E04,_mRNA_sequence |
| 183 | gi\|29469853\|gb\|GW781995.1\|GW781995_PBC041D06_06423_Parthenium_ |
| | argentatum_cold_acclimated_bark_cDNA_library_(PBC)_Parthenium_argentatum_ |
| | cDNA_clone_PBC041D06,_mRNA_sequence |
| 184 | gi\|294698526\|gb\|GW781990.1\|GW781990_PBC041D04_06418_Parthenium_ |
| | argentatum_cold_acclimated_bark_cDNA_library_(PBC)_Parthenium_argentatum_ |
| | cDNA_clone_PBC041D04,_mRNA_sequence |
| 185 | gi\|294698519\|gb\|GW781983.1\|GW781983_PBC041C06_06411_Parthenium_ |
| | argentatum_cold_acclimated_bark_cDNA_library_(PBC)_Parthenium_argentatum_ |
| | cDNA_clone_PBC041C06,_mRNA_sequence |
| 186 | gi\|294698504\|gb\|GW781968.1\|GW781968_PBC041B04_06396_Parthenium_ |
| | argentatum_cold_acclimated_bark_cDNA_library_(PBC)_Parthenium_argentatum_ |
| | cDNA_clone_PBC041B04,_mRNA_sequence |
| 187 | gi\|294698500\|gb\|GW781964.1\|GW781964_PBC041A11_06392_Parthenium_ |
| | argentatum_cold_acclimated_bark_cDNA_library_(PBC)_Parthenium_argentatum_ |
| | cDNA_clone_PBC041A11,_mRNA_sequence |
| 188 | gi\|294698479\|gb\|GW781943.1\|GW781943_PBC040H04_06371_Parthenium_ |
| | argentatum_cold_acclimated_bark_cDNA_library_(PBC)_Parthenium_argentatum_ |
| | cDNA_clone_PBC040H04,_mRNA_sequence |
| 189 | gi\|294698440\|gb\|GW776295.1\|GW776295_PAB909e01_00723_Parthenium_ |
| | argentatum_cold_acclimated_bark_cDNA_library_(PAB)_Parthenium_argentatum_ |
| | cDNA_clone_PAB909e01,_mRNA_sequence |
| 190 | gi\|294698291\|gb\|GW781916.1\|GW781916_PBC040E09_06344_Parthenium_ |
| | argentatum_cold_acclimated_bark_cDNA_library_(PBC)_Parthenium_argentatum_ |
| | cDNA_clone_PBC040E09,_mRNA_sequence |
| 191 | gi\|294698273\|gb\|GW781898.1\|GW781898_PBC040D04_06326_Parthenium_ |
| | argentatum_cold_acclimated_bark_cDNA_library_(PBC)_Parthenium_argentatum_ |
| | cDNA_clone_PBC040D04,_mRNA_sequence |
| 192 | gi\|294698242\|gb\|GW781867.1\|GW781867_PBC040A06_06295_Parthenium_ |
| | argentatum_cold_acclimated_bark_cDNA_library_(PBC)_Parthenium_argentatum_ |
| | cDNA_clone_PBC040A06,_mRNA_sequence |
| 193 | gi\|294698242\|gb\|GW781867.1\|GW781867_PBC040A06_06295_Parthenium_ |
| | argentatum_cold_acclimated_bark_cDNA_library_(PBC)_Parthenium_argentatum_ |
| | cDNA_clone_PBC040A06,_mRNA_sequence |
| 194 | gi\|294698191\|gb\|GW776207.1\|GW776207_PAB908a04_00635_Parthenium_ |
| | argentatum_cold_acclimated_bark_cDNA_library_(PAB)_Parthenium_argentatum_ |
| | cDNA_clone_PAB908a04,_mRNA_sequence |
| 195 | gi\|294698154\|gb\|GW776170.1\|GW776170_PAB908g04_00598_Parthenium_ |
| | argentatum_cold_acclimated_bark_cDNA_library_(PAB)_Parthenium_argentatum_ |
| | cDNA_clone_PAB908g04,_mRNA_sequence |
| 196 | gi\|294698047\|gb\|GW781833.1\|GW781833_PBC039F06_06261_Parthenium_ |
| | argentatum_cold_acclimated_bark_cDNA_library_(PBC)_Parthenium_argentatum_ |
| | cDNA_clone_PBC039F06,_mRNA_sequence |
| 197 | gi\|294698028\|gb\|GW781814.1\|GW781814_PBC039D10_06242_Parthenium_ |
| | argentatum_cold_acclimated_bark_cDNA_library_(PBC)_Parthenium_argentatum_ |
| | cDNA_clone_PBC039D10,_mRNA_sequence |
| 198 | gi\|294697958\|gb\|GW776135.1\|GW776135_PAB907c12_00563_Parthenium_ |
| | argentatum_cold_acclimated_bark_cDNA_library_(PAB)_Parthenium_argentatum_ |
| | cDNA_clone_PAB907c12,_mRNA_sequence |
| 199 | gi\|294697949\|gb\|GW776126.1\|GW776126_PAB907g01_00554_Parthenium_ |
| | argentatum_cold_acclimated_bark_cDNA_library_(PAB)_Parthenium_argentatum_ |
| | cDNA_clone_PAB907g01,_mRNA_sequence |
| 200 | gi\|294697929\|gb\|GW776106.1\|GW776106_PAB907g09_00534_Parthenium_ |
| | argentatum_cold_acclimated_bark_cDNA_library_(PAB)_Parthenium_argentatum_ |
| | cDNA_clone_PAB907g09,_mRNA_sequence |
| 201 | gi\|294697925\|gb\|GW776102.1\|GW776102_PAB907c09_00530_Parthenium_ |
| | argentatum_cold_acclimated_bark_cDNA_library_(PAB)_Parthenium_argentatum_ |
| | cDNA_clone_PAB907c09,_mRNA_sequence |
| 202 | gi\|294697887\|gb\|GW779224.1\|GW779224_PBC010H10_03652_Parthenium_ |
| | argentatum_cold_acclimated_bark_cDNA_library_(PBC)_Parthenium_argentatum_ |
| | cDNA_clone_PBC010H10,_mRAN_sequence |
| 203 | gi\|294697774\|gb\|GW779111.1\|GW779111_PBC009F10_03539_Parthenium_ |
| | argentatum_cold_acclimated_bark_cDNA_library_(PBC)_Parthenium_argentatum_ |
| | cDNA_clone_PBC009F10,_mRAN_sequence |
| 204 | gi\|294697771\|gb\|GW779108.1\|GW7791108_PBC009F07_03536_Parthenium_ |
| | argentatum_cold_acclimated_bark_cDNA_library_(PBC)_Parthenium_argentatum_ |
| | cDNA_clone_PBC009F07,_mRAN_sequence |
| 205 | gi\|294697768\|gb\|GW779105.1\|GW779105_PBC009F04_03533_Parthenium_ |
| | argentatum_cold_acclimated_bark_cDNA_library_(PBC)_Parthenium_argentatum_ |
| | cDNA_clone_PBC009F04,_mRAN_sequence |
| 206 | gi\|294697755\|gb\|GW779092.1\|GW779092_PBC009E03_03520_Parthenium_ |
| | argentatum_cold_acclimated_bark_cDNA_library_(PBC)_Parthenium_argentatum_ |
| | cDNA_clone_PBC009E03,_mRAN_sequence |
| 207 | gi\|294697707\|gb\|GW779044.1\|GW779044_PBC008H12_03472_Parthenium_ |
| | argentatum_cold_acclimated_bark_cDNA_library_(PBC)_Parthenium_argentatum_ |
| | cDNA_clone_PBC008H12,_mRAN_sequence |
| 208 | gi\|294697698\|gb\|GW779035.1\|GW779035_PBC008H03_03463_Parthenium_ |
| | argentatum_cold_acclimated_bark_cDNA_library_(PBC)_Parthenium_argentatum_ |
| | cDNA_clone_PBC008H03,_mRAN_sequence |
| 209 | gi\|294697697\|gb\|GW779034.1\|GW779034_PBC008H01_03462_Parthenium_ |
| | argentatum_cold_acclimated_bark_cDNA_library_(PBC)_Parthenium_argentatum_ |
| | cDNA_clone_PBC008H01,_mRAN_sequence |
| 210 | gi\|294697689\|gb\|GW779026.1\|GW779026_PBC008G05_03454_Parthenium_ |
| | argentatum_cold_acclimated_bark_cDNA_library_(PBC)_Parthenium_argentatum_ |
| | cDNA_clone_PBC008G05,_mRAN_sequence |
| 211 | gi\|294697673\|gb\|GW779010.1\|GW779010_PBC008E12_03438_Parthenium_ |
| | argentatum_cold_acclimated_bark_cDNA_library_(PBC)_Parthenium_argentatum_ |
| | cDNA_clone_PBC008E12,_mRAN_sequence |
| 212 | gi\|294697669\|gb\|GW779006.1\|GW779006_PBC008E08_03434_Parthenium_ |
| | argentatum_cold_acclimated_bark_cDNA_library_(PBC)_Parthenium_argentatum_ |
| | cDNA_clone_PBC008E08,_mRAN_sequence |
| 213 | gi\|294697629\|gb\|GW778966.1\|GW778966_PBC008A12_03394_Parthenium_ |
| | argentatum_cold_acclimated_bark_cDNA_library_(PBC)_Parthenium_argentatum_ |
| | cDNA_clone_PBC008A12,_mRAN_sequence |
| 214 | gi\|294697628\|gb\|GW778965.1\|GW778965_PBC008A11_03393_Parthenium_ |
| | argentatum_cold_acclimated_bark_cDNA_library_(PBC)_Parthenium_argentatum_ |
| | cDNA_clone_PBC008A11,_mRAN_sequence |
| 215 | gi\|294697562\|gb\|GW778899.1\|GW778899_PBC007C12_03327_Parthenium_ |
| | argentatum_cold_acclimated_bark_cDNA_library_(PBC)_Parthenium_argentatum_ |
| | cDNA_clone_PBC007C12,_mRAN_sequence |
| 216 | gi\|294697557\|gb\|GW778894.1\|GW778894_PBC007C06_03322_Parthenium_ |
| | argentatum_cold_acclimated_bark_cDNA_library_(PBC)_Parthenium_argentatum_ |
| | cDNA_clone_PBC007C06,_mRAN_sequence |
| 217 | gi\|294697540\|gb\|GW778877.1\|GW778877_PBC007A12_03305_Parthenium_ |
| | argentatum_cold_acclimated_bark_cDNA_library_(PBC)_Parthenium_argentatum_ |
| | cDNA_clone_PBC007A12,_mRAN_sequence |
| 218 | gi\|294697497\|gb\|GW778834.1\|GW778834_PBC006F04_03262_Parthenium_ |
| | argentatum_cold_acclimated_bark_cDNA_library_(PBC)_Parthenium_argentatum_ |
| | cDNA_clone_PBC006F04,_mRAN_sequence |
| 219 | gi\|294697442\|gb\|GW778779.1\|GW778779_PBC005H12_03207_Parthenium_ |
| | argentatum_cold_acclimated_bark_cDNA_library_(PBC)_Parthenium_argentatum_ |
| | cDNA_clone_PBC005H12,_mRAN_sequence |
| 220 | gi\|294697437\|gb\|GW778774.1\|GW778774_PBC005H07_03202_Parthenium_ |
| | argentatum_cold_acclimated_bark_cDNA_library_(PBC)_Parthenium_argentatum_ |
| | cDNA_clone_PBC005H07,_mRAN_sequence |
| 221 | gi\|294697368\|gb\|GW778705.1\|GW778705_PBC005B08_03133_Parthenium_ |
| | argentatum_cold_acclimated_bark_cDNA_library_(PBC)_Parthenium_argentatum_ |
| | cDNA_clone_PBC005B08,_mRAN_sequence |
| 222 | gi\|294697364\|gb\|GW778701.1\|GW778701_PBC005B04_03129_Parthenium_ |
| | argentatum_cold_acclimated_bark_cDNA_library_(PBC)_Parthenium_argentatum_ |
| | cDNA_clone_PBC005B04,_mRAN_sequence |
| 223 | gi\|294697342\|gb\|GW778679.1\|GW778679_PBC004H06_03107_Parthenium_ |
| | argentatum_cold_acclimated_bark_cDNA_library_(PBC)_Parthenium_argentatum_ |
| | cDNA_clone_PBC004H06,_mRAN_sequence |
| 224 | gi\|294697329\|gb\|GW778666.1\|GW778666_PBC004G03_03094_Parthenium_ |
| | argentatum_cold_acclimated_bark_cDNA_library_(PBC)_Parthenium_argentatum_ |
| | cDNA_clone_PBC004G03,_mRAN_sequence |
| 225 | gi\|294697324\|gb\|GW778661.1\|GW778661_PBC004F10_03089_Parthenium_ |
| | argentatum_cold_acclimated_bark_cDNA_library_(PBC)_Parthenium_argentatum_ |
| | cDNA_clone_PBC004F10,_mRAN_sequence |
| 226 | gi\|294697310\|gb\|GW778647.1\|GW778647_PBC004E06_03075_Parthenium_ |
| | argentatum_cold_acclimated_bark_cDNA_library_(PBC)_Parthenium_argentatum_ |
| | cDNA_clone_PBC004E06,_mRAN_sequence |
| 227 | gi\|294697287\|gb\|GW778624.1\|GW778624_PBC004C05_03052_Parthenium_ |
| | argentatum_cold_acclimated_bark_cDNA_library_(PBC)_Parthenium_argentatum_ |
| | cDNA_clone_PBC004C05,_mRAN_sequence |
| 228 | gi\|294697189\|gb\|GW787305.1\|GW787305_PBC100H06_11733_Parthenium_ |
| | argentatum_cold_acclimated_bark_cDNA_library_(PBC)_Parthenium_argentatum_ |
| | cDNA_clone_PBC100H06,_mRAN_sequence |
| 229 | gi\|294697098\|gb\|GW787296.1\|GW787296_PBC100G08_11724_Parthenium_ |
| | argentatum_cold_acclimated_bark_cDNA_library_(PBC)_Parthenium_argentatum_ |
| | cDNA_clone_PBC100G08,_mRAN_sequence |
| 230 | gi\|294697081\|gb\|GW787279.1\|GW787279_PBC100F05_11707_Parthenium_ |
| | argentatum_cold_acclimated_bark_cDNA_library_(PBC)_Parthenium_argentatum_ |
| | cDNA_clone_PBC100F05,_mRAN_sequence |
| 231 | gi\|294697076\|gb\|GW787274.1\|GW787274_PBC100E08_11702_Parthenium_ |
| | argentatum_cold_acclimated_bark_cDNA_library_(PBC)_Parthenium_argentatum_ |
| | cDNA_clone_PBC100E08,_mRAN_sequence |
| 232 | gi\|294697050\|gb\|GW787248.1\|GW787248_PBC100C05_11676_Parthenium_ |
| | argentatum_cold_acclimated_bark_cDNA_library_(PBC)_Parthenium_argentatum_ |
| | cDNA_clone_PBC100C05,_mRAN_sequence |
| 233 | gi\|294696944\|gb\|GW787224.1\|GW787224_PBC100A05_11652_Parthenium_ |
| | argentatum_cold_acclimated_bark_cDNA_library_(PBC)_Parthenium_argentatum_ |
| | cDNA_clone_PBC100A05,_mRAN_sequence |
| 234 | gi\|294696926\|gb\|GW787206.1\|GW787206_PBC099G06_11634_Parthenium_ |
| | argentatum_cold_acclimated_bark_cDNA_library_(PBC)_Parthenium_argentatum_ |
| | cDNA_clone_PBC099G06,_mRAN_sequence |
| 235 | gi\|294696781\|gb\|GW787143.1\|GW787143_PBC099A10_11571_Parthenium_ |
| | argentatum_cold_acclimated_bark_cDNA_library_(PBC)_Parthenium_argentatum_ |
| | cDNA_clone_PBC099A10,_mRAN_sequence |
| 236 | gi\|294696622\|gb\|GW787066.1\|GW787066_PBC098C05_11494_Parthenium_ |
| | argentatum_cold_acclimated_bark_cDNA_library_(PBC)_Parthenium_argentatum_ |
| | cDNA_clone_PBC098C05,_mRAN_sequence |
| 237 | gi\|294696582\|gb\|GW787026.1\|GW787026_PBC097G09_11454_Parthenium_ |
| | argentatum_cold_acclimated_bark_cDNA_library_(PBC)_Parthenium_argentatum_ |
| | cDNA_clone_PBC097G09,_mRAN_sequence |
| 238 | gi\|294696398\|gb\|GW786935.1\|GW786935_PBC096G06_11363_Parthenium_ |
| | argentatum_cold_acclimated_bark_cDNA_library_(PBC)_Parthenium_argentatum_ |
| | cDNA_clone_PBC096G06,_mRAN_sequence |
| 239 | gi\|294696387\|gb\|GW786924.1\|GW786924_PBC096F05_11352_Parthenium_ |
| | argentatum_cold_acclimated_bark_cDNA_library_(PBC)_Parthenium_argentatum_ |
| | cDNA_clone_PBC096F05,_mRAN_sequence |
| 240 | gi\|294696284\|gb\|GW786903.1\|GW786903_PBC096D06_11331_Parthenium_ |
| | argentatum_cold_acclimated_bark_cDNA_library_(PBC)_Parthenium_argentatum_ |
| | cDNA_clone_PBC096D06,_mRAN_sequence |
| 241 | gi\|294696221\|gb\|GW786840.1\|GW786840_PBC095F09_11268_Parthenium_ |
| | argentatum_cold_acclimated_bark_cDNA_library_(PBC)_Parthenium_argentatum_ |
| | cDNA_clone_PBC095F09,_mRAN_sequence |
| 242 | gi\|294696103\|gb\|GW786804.1\|GW786804_PBC095C07_11232_Parthenium_ |
| | argentatum_cold_acclimated_bark_cDNA_library_(PBC)_Parthenium_argentatum_ |
| | cDNA_clone_PBC095C07,_mRAN_sequence |
| 243 | gi\|294695793\|gb\|GW784921.1\|GW784921_PBC074B12_09349_Parthenium_ |
| | argentatum_cold_acclimated_bark_cDNA_library_(PBC)_Parthenium_argentatum_ |
| | cDNA_clone_PBC074B12,_mRAN_sequence |
| 244 | gi\|294695793\|gb\|GW784921.1\|GW784921_PBC074B12_09349_Parthenium_ |
| | argentatum_cold_acclimated_bark_cDNA_library_(PBC)_Parthenium_argentatum_ |
| | cDNA_clone_PBC074B12,_mRAN_sequence |
| 245 | gi\|294695786\|gb\|GW784914.1\|GW784914_PBC074B05_09342_Parthenium_ |
| | argentatum_cold_acclimated_bark_cDNA_library_(PBC)_Parthenium_argentatum_ |
| | cDNA_clone_PBC074B05,_mRAN_sequence |
| 246 | gi\|294695778\|gb\|GW784906.1\|GW784906_PBC074A09_09334_Parthenium_ |
| | argentatum_cold_acclimated_bark_cDNA_library_(PBC)_Parthenium_argentatum_ |
| | cDNA_clone_PBC074A09,_mRAN_sequence |
| 247 | gi\|294695776\|gb\|GW784904.1\|GW784904_PBC074A07_09332_Parthenium_ |
| | argentatum_cold_acclimated_bark_cDNA_library_(PBC)_Parthenium_argentatum_ |
| | cDNA_clone_PBC074A07,_mRAN_sequence |
| 248 | gi\|294695740\|gb\|GW784868.1\|GW784868_PBC073F05_09296_Parthenium_ |
| | argentatum_cold_acclimated_bark_cDNA_library_(PBC)_Parthenium_argentatum_ |
| | cDNA_clone_PBC073F05,_mRAN_sequence |
| 249 | gi\|294695446\|gb\|GW784818.1\|GW784818_PBC073A10_09246_Parthenium_ |
| | argentatum_cold_acclimated_bark_cDNA_library_(PBC)_Parthenium_argentatum_ |
| | cDNA_clone_PBC073A10,_mRAN_sequence |
| 250 | gi\|294695407\|gb\|GW784779.1\|GW784779_PBC072F06_09207_Parthenium_ |
| | argentatum_cold_acclimated_bark_cDNA_library_(PBC)_Parthenium_argentatum_ |
| | cDNA_clone_PBC072F06,_mRAN_sequence |
| 251 | gi\|294695104\|gb\|GW784720.1\|GW784720_PBC072A05_09148_Parthenium_ |
| | argentatum_cold_acclimated_bark_cDNA_library_(PBC)_Parthenium_argentatum_ |
| | cDNA_clone_PBC072A05,_mRAN_sequence |
| 252 | gi\|294694836\|gb\|GW784696.1\|GW784696_PBC071G05_09124_Parthenium_ |
| | argentatum_cold_acclimated_bark_cDNA_library_(PBC)_Parthenium_argentatum_ |
| | cDNA_clone_PBC071G05,_mRAN_sequence |
| 253 | gi\|294694818\|gb\|GW784678.1\|GW784678_PBC071E05_09106_Parthenium_ |
| | argentatum_cold_acclimated_bark_cDNA_library_(PBC)_Parthenium_argentatum_ |
| | cDNA_clone_PBC071E05,_mRAN_sequence |
| 254 | gi\|294694776\|gb\|GW784636.1\|GW784636_PBC071A07_09064_Parthenium_ |
| | argentatum_cold_acclimated_bark_cDNA_library_(PBC)_Parthenium_argentatum_ |
| | cDNA_clone_PBC071A07,_mRAN_sequence |
| 255 | gi\|294694457\|gb\|GW784561.1\|GW784561_PBC070B11_08989_Parthenium_ |
| | argentatum_cold_acclimated_bark_cDNA_library_(PBC)_Parthenium_argentatum_ |
| | cDNA_clone_PBC070B11,_mRAN_sequence |
| 256 | gi\|294694259\|gb\|GW784527.1\|GW784527_PBC069H05_09064_Parthenium_ |
| | argentatum_cold_acclimated_bark_cDNA_library_(PBC)_Parthenium_argentatum_ |
| | cDNA_clone_PBC069H05,_mRAN_sequence |
| 257 | gi\|294694247\|gb\|GW784515.1\|GW784515_PBC069F12_08943_Parthenium_ |
| | argentatum_cold_acclimated_bark_cDNA_library_(PBC)_Parthenium_argentatum_ |
| | cDNA_clone_PBC069F12,_mRAN_sequence |
| 258 | gi\|294694234\|gb\|GW784502.1\|GW784502_PBC069E10_08930_Parthenium_ |
| | argentatum_cold_acclimated_bark_cDNA_library_(PBC)_Parthenium_argentatum_ |
| | cDNA_clone_PBC069E10,_mRAN_sequence |
| 259 | gi\|294694230\|gb\|GW784498.1\|GW784498_PBC069E06_08926_Parthenium_ |
| | argentatum_cold_acclimated_bark_cDNA_library_(PBC)_Parthenium_argentatum_ |
| | cDNA_clone_PBC069E06,_mRAN_sequence |
| 260 | gi\|294694215\|gb\|GW784483.1\|GW784483_PBC069D05_08911_Parthenium_ |
| | argentatum_cold_acclimated_bark_cDNA_library_(PBC)_Parthenium_argentatum_ |
| | cDNA_clone_PBC069D05,_mRAN_sequence |
| 261 | gi\|294694201\|gb\|GW784469.1\|GW784469_PBC069B10_08897_Parthenium_ |
| | argentatum_cold_acclimated_bark_cDNA_library_(PBC)_Parthenium_argentatum_ |
| | cDNA_clone_PBC069B10_mRNA_sequence |
| 262 | gi\|294693983\|gb\|GW784415.1\|GW784415_PBC068F05_08843_Parthenium_ |
| | argentatum_cold_acclimated_bark_cDNA_library_(PBC)_Parthenium_argentatum_ |
| | cDNA_clone_PBC068F05,_mRNA_sequence |
| 263 | gi\|294693755\|gb\|GW784351.1\|GW784351_PBC067H06_08879_Parthenium_ |
| | argentatum_cold_acclimated_bark_cDNA_library_(PBC)_Parthenium_argentatum_ |
| | cDNA_clone_PBC067H06,_mRNA_sequence |
| 264 | gi\|294693731\|gb\|GW784327.1\|GW784327_PBC067F05_08755_Parthenium_ |
| | argentatum_cold_acclimated_bark_cDNA_library_(PBC)_Parthenium_argentatum_ |
| | cDNA_clone_PBC067F05,_mRNA_sequence |
| 265 | gi\|294693719\|gb\|GW784315.1\|GW784315_PBC067E05_08743_Parthenium_ |
| | argentatum_cold_acclimated_bark_cDNA_library_(PBC)_Parthenium_argentatum_ |
| | cDNA_clone_PBC067F05,_mRNA_sequence |
| 266 | gi\|294693716\|gb\|GW784312.1\|GW784312_PBC067E05_08740_Parthenium_ |
| | argentatum_cold_acclimated_bark_cDNA_library_(PBC)_Parthenium_argentatum_ |
| | cDNA_clone_PBC067E05,_mRNA_sequence |
| 267 | gi\|294693464\|gb\|GW781774.1\|GW781774_PBC039A03_06202_Parthenium_ |
| | argentatum_cold_acclimated_bark_cDNA_library_(PBC)_Parthenium_argentatum_ |
| | cDNA_clone_PBC039A03,_mRNA_sequence |
| 268 | gi\|294693447\|gb\|GW781757.1\|GW781757_PBC038G08_06185_Parthenium_ |
| | argentatum_cold_acclimated_bark_cDNA_library_(PBC)_Parthenium_argentatum_ |
| | cDNA_clone_PBC038G08,_mRNA_sequence |
| 269 | gi\|294693432\|gb\|GW781742.1\|GW781742_PBC038F04_06170_Parthenium_ |
| | argentatum_cold_acclimated_bark_cDNA_library_(PBC)_Parthenium_argentatum_ |
| | cDNA_clone_PBC038F04,_mRNA_sequence |
| 270 | gi\|294693397\|gb\|GW781707.1\|GW781707_PBC038C03_06135_Parthenium_ |
| | argentatum_cold_acclimated_bark_cDNA_library_(PBC)_Parthenium_argentatum_ |
| | cDNA_clone_PBC038C03,_mRNA_sequence |
| 271 | gi\|294693128\|gb\|GW781680.1\|GW781680_PBC037H10_06108_Parthenium_ |
| | argentatum_cold_acclimated_bark_cDNA_library_(PBC)_Parthenium_argentatum_ |
| | cDNA_clone_PBC037H10,_mRNA_sequence |
| 272 | gi\|294692815\|gb\|GW781609.1\|GW781609_PBC037B07_06037_Parthenium_ |
| | argentatum_cold_acclimated_bark_cDNA_library_(PBC)_Parthenium_argentatum_ |
| | cDNA_clone_PBC037B07,_mRNA_sequence |
| 273 | gi\|294692811\|gb\|GW781605.1\|GW781605_PBC037B03_06033_Parthenium_ |
| | argentatum_cold_acclimated_bark_cDNA_library_(PBC)_Parthenium_argentatum_ |
| | cDNA_clone_PBC037B03,_mRNA_sequence |
| 274 | gi\|294692767\|gb\|GW781561.1\|GW781561_PBC036F03_05989_Parthenium_ |
| | argentatum_cold_acclimated_bark_cDNA_library_(PBC)_Parthenium_argentatum_ |
| | cDNA_clone_PBC036F03,_mRNA_sequence |
| 275 | gi\|294692765\|gb\|GW781559.1\|GW781559_PBC036E11_05987_Parthenium_ |
| | argentatum_cold_acclimated_bark_cDNA_library_(PBC)_Parthenium_argentatum_ |
| | cDNA_clone_PBC036E11,_mRNA_sequence |
| 276 | gi\|294692762\|gb\|GW781556.1\|GW781556_PBC036E08_05984_Parthenium_ |
| | argentatum_cold_acclimated_bark_cDNA_library_(PBC)_Parthenium_argentatum_ |
| | cDNA_clone_PBC036E08,_mRNA_sequence |
| 277 | gi\|294692751\|gb\|GW781545.1\|GW781545_PBC036D08_05973_Parthenium_ |
| | argentatum_cold_acclimated_bark_cDNA_library_(PBC)_Parthenium_argentatum_ |
| | cDNA_clone_PBC036D08,_mRNA_sequence |
| 278 | gi\|294692496\|gb\|GW781532.1\|GW781532_PBC036C07_05960_Parthenium_ |
| | argentatum_cold_acclimated_bark_cDNA_library_(PBC)_Parthenium_argentatum_ |
| | cDNA_clone_PBC036C07,_mRNA_sequence |
| 279 | gi\|294692491\|gb\|GW781527.1\|GW781527_PBC036C03_05955_Parthenium_ |
| | argentatum_cold_acclimated_bark_cDNA_library_(PBC)_Parthenium_argentatum_ |
| | cDNA_clone_PBC036C03,_mRNA_sequence |
| 280 | gi\|294692484\|gb\|GW781520.1\|GW781520_PBC036B07_05948_Parthenium_ |
| | argentatum_cold_acclimated_bark_cDNA_library_(PBC)_Parthenium_argentatum_ |
| | cDNA_clone_PBC036B07,_mRNA_sequence |
| 281 | gi\|294692473\|gb\|GW781509.1\|GW781509_PBC036A08_05937_Parthenium_ |
| | argentatum_cold_acclimated_bark_cDNA_library_(PBC)_Parthenium_argentatum_ |
| | cDNA_clone_PBC036A08,_mRNA_sequence |
| 282 | gi\|294692448\|gb\|GW781484.1\|GW781484_PBC035G06_05912_Parthenium_ |
| | argentatum_cold_acclimated_bark_cDNA_library_(PBC)_Parthenium_argentatum_ |
| | cDNA_clone_PBC035G06,_mRNA_sequence |
| 283 | gi\|294692168\|gb\|GW781446.1\|GW781446_PBC035D03_05874_Parthenium_ |
| | argentatum_cold_acclimated_bark_cDNA_library_(PBC)_Parthenium_argentatum_ |
| | cDNA_clone_PBC035D03,_mRNA_sequence |
| 284 | gi\|294692163\|gb\|GW781441.1\|GW781441_PBC035C10_05869_Parthenium_ |
| | argentatum_cold_acclimated_bark_cDNA_library_(PBC)_Parthenium_argentatum_ |
| | cDNA_clone_PBC035C10,_mRNA_sequence |
| 285 | gi\|294692155\|gb\|GW781433.1\|GW781433_PBC035C03_05861_Parthenium_ |
| | argentatum_cold_acclimated_bark_cDNA_library_(PBC)_Parthenium_argentatum_ |
| | cDNA_clone_PBC035C03,_mRNA_sequence |
| 286 | gi\|294691855\|gb\|GW781375.1\|GW781375_PBC034F03_05803_Parthenium_ |
| | argentatum_cold_acclimated_bark_cDNA_library_(PBC)_Parthenium_argentatum_ |
| | cDNA_clone_PBC034F03,_mRNA_sequence |
| 287 | gi\|294691825\|gb\|GW781345.1\|GW781345_PBC034C06_05773_Parthenium_ |
| | argentatum_cold_acclimated_bark_cDNA_library_(PBC)_Parthenium_argentatum_ |
| | cDNA_clone_PBC034C06,_mRNA_sequence |
| 288 | gi\|294691811\|gb\|GW781331.1\|GW781331_PBC034B03_05759_Parthenium_ |
| | argentatum_cold_acclimated_bark_cDNA_library_(PBC)_Parthenium_argentatum_ |
| | cDNA_clone_PBC034B03,_mRNA_sequence |
| 289 | gi\|294691793\|gb\|GW781313.1\|GW781313_PBC033H08_05741_Parthenium_ |
| | argentatum_cold_acclimated_bark_cDNA_library_(PBC)_Parthenium_argentatum_ |
| | cDNA_clone_PBC033H08,_mRNA_sequence |
| 290 | gi\|294691616\|gb\|GW781290.1\|GW781290_PBC033F09_05718_Parthenium_ |
| | argentatum_cold_acclimated_bark_cDNA_library_(PBC)_Parthenium_argentatum_ |
| | cDNA_clone_PBC033F09,_mRNA_sequence |
| 291 | gi\|294691599\|gb\|GW781273.1\|GW781273_PBC033E03_05701_Parthenium_ |
| | argentatum_cold_acclimated_bark_cDNA_library_(PBC)_Parthenium_argentatum_ |
| | cDNA_clone_PBC033E03,_mRNA_sequence |
| 292 | gi\|294691565\|gb\|GW781239.1\|GW781239_PBC033A11_05667_Parthenium_ |
| | argentatum_cold_acclimated_bark_cDNA_library_(PBC)_Parthenium_argentatum_ |
| | cDNA_clone_PBC033A11,_mRNA_sequence |
| 293 | gi\|294691383\|gb\|GW781219.1\|GW781219_PBC032H03_05647_Parthenium_ |
| | argentatum_cold_acclimated_bark_cDNA_library_(PBC)_Parthenium_argentatum_ |
| | cDNA_clone_PBC032H03,_mRNA_sequence |
| 294 | gi\|294691382\|gb\|GW781218.1\|GW781218_PBC032G12_05646_Parthenium_ |
| | argentatum_cold_acclimated_bark_cDNA_library_(PBC)_Parthenium_argentatum_ |
| | cDNA_clone_PBC032G12,_mRNA_sequence |
| 295 | gi\|294691321\|gb\|GW781157.1\|GW781157_PBC032B03_05585_Parthenium_ |
| | argentatum_cold_acclimated_bark_cDNA_library_(PBC)_Parthenium_argentatum_ |
| | cDNA_clone_PBC032B03,_mRNA_sequence |
| 296 | gi\|294691208\|gb\|GW784286.1\|GW784286_PBC067B11_08714_Parthenium_ |
| | argentatum_cold_acclimated_bark_cDNA_library_(PBC)_Parthenium_argentatum_ |
| | cDNA_clone_PBC067B11,_mRNA_sequence |
| 297 | gi\|294691197\|gb\|GW784275.1\|GW784275_PBC067A12_08703_Parthenium_ |
| | argentatum_cold_acclimated_bark_cDNA_library_(PBC)_Parthenium_argentatum_ |
| | cDNA_clone_PBC067A12,_mRNA_sequence |
| 298 | gi\|294691186\|gb\|GW784264.1\|GW784264_PBC067A05_08692_Parthenium_ |
| | argentatum_cold_acclimated_bark_cDNA_library_(PBC)_Parthenium_argentatum_ |
| | cDNA_clone_PBC067A05,_mRNA_sequence |
| 299 | gi\|294691165\|gb\|GW784243.1\|GW784243_PBC066G05_08671_Parthenium_ |
| | argentatum_cold_acclimated_bark_cDNA_library_(PBC)_Parthenium_argentatum_ |
| | cDNA_clone_PBC066G05,_mRNA_sequence |
| 300 | gi\|294691156\|gb\|GW784234.1\|GW784234_PBC066F06_08662_Parthenium_ |
| | argentatum_cold_acclimated_bark_cDNA_library_(PBC)_Parthenium_argentatum_ |
| | cDNA_clone_PBC066F06,_mRNA_sequence |
| 301 | gi\|294691052\|gb\|GW784212.1\|GW784212_PBC066D07_08640_Parthenium_ |
| | argentatum_cold_acclimated_bark_cDNA_library_(PBC)_Parthenium_argentatum_ |
| | cDNA_clone_PBC066D07,_mRNA_sequence |
| 302 | gi\|294691030\|gb\|GW784190.1\|GW784190_PBC066B07_08618_Parthenium_ |
| | argentatum_cold_acclimated_bark_cDNA_library_(PBC)_Parthenium_argentatum_ |
| | cDNA_clone_PBC066B07,_mRNA_sequence |
| 303 | gi\|294691024\|gb\|GW784184.1\|GW784184_PBC066B05_08612_Parthenium_ |
| | argentatum_cold_acclimated_bark_cDNA_library_(PBC)_Parthenium_argentatum_ |
| | cDNA_clone_PBC066B05,_mRNA_sequence |
| 304 | gi\|294691008\|gb\|GW784168.1\|GW784168_PBC065H09_08596_Parthenium_ |
| | argentatum_cold_acclimated_bark_cDNA_library_(PBC)_Parthenium_argentatum_ |
| | cDNA_clone_PBC065H09,_mRNA_sequence |
| 305 | gi\|294691008\|gb\|GW784168.1\|GW784168_PBC065H09_08596_Parthenium_ |
| | argentatum_cold_acclimated_bark_cDNA_library_(PBC)_Parthenium_argentatum_ |
| | cDNA_clone_PBC065H09,_mRNA_sequence |
| 306 | gi\|294691004\|gb\|GW784164.1\|GW784164_PBC065H05_08592_Parthenium_ |
| | argentatum_cold_acclimated_bark_cDNA_library_(PBC)_Parthenium_argentatum_ |
| | cDNA_clone_PBC065H05,_mRNA_sequence |
| 307 | gi\|294690989\|gb\|GW784149.1\|GW784149_PBC065F12_08577_Parthenium_ |
| | argentatum_cold_acclimated_bark_cDNA_library_(PBC)_Parthenium_argentatum_ |
| | cDNA_clone_PBC065F12,_mRNA_sequence |
| 308 | gi\|294690894\|gb\|GW784136.1\|GW784136_PBC065E11_08564_Parthenium_ |
| | argentatum_cold_acclimated_bark_cDNA_library_(PBC)_Parthenium_argentatum_ |
| | cDNA_clone_PBC067H06,_mRNA_sequence |
| 309 | gi\|294690893\|gb\|GW784135.1\|GW784135_PBC065E09_08563_Parthenium_ |
| | argentatum_cold_acclimated_bark_cDNA_library_(PBC)_Parthenium_argentatum_ |
| | cDNA_clone_PBC065E09,_mRNA_sequence |
| 310 | gi\|294690880\|gb\|GW784122.1\|GW784122_PBC065D07_08550_Parthenium_ |
| | argentatum_cold_acclimated_bark_cDNA_library_(PBC)_Parthenium_argentatum_ |
| | cDNA_clone_PBC065D07,_mRNA_sequence |
| 311 | gi\|294690868\|gb\|GW784110.1\|GW784110_PBC065C06_08538_Parthenium_ |
| | argentatum_cold_acclimated_bark_cDNA_library_(PBC)_Parthenium_argentatum_ |
| | cDNA_clone_PBC065C06,_mRNA_sequence |
| 312 | gi\|294690856\|gb\|GW784098.1\|GW7844098_PBC065B06_08526_Parthenium_ |
| | argentatum_cold_acclimated_bark_cDNA_library_(PBC)_Parthenium_argentatum_ |
| | cDNA_clone_PBC065B06,_mRNA_sequence |
| 313 | gi\|294690839\|gb\|GW784081.1\|GW784081_PBC065A05_08509_Parthenium_ |
| | argentatum_cold_acclimated_bark_cDNA_library_(PBC)_Parthenium_argentatum_ |
| | cDNA_clone_PBC065A05,_mRNA_sequence |
| 314 | gi\|294690693\|gb\|GW784017.1\|GW784017_PBC064C08_08445_Parthenium_ |
| | argentatum_cold_acclimated_bark_cDNA_library_(PBC)_Parthenium_argentatum_ |
| | cDNA_clone_PBC064C08,_mRNA_sequence |
| 315 | gi\|294690682\|gb\|GW784006.1\|GW784006_PBC064B05_08434_Parthenium_ |
| | argentatum_cold_acclimated_bark_cDNA_library_(PBC)_Parthenium_argentatum_ |
| | cDNA_clone_PBC064B05,_mRNA_sequence |
| 316 | gi\|294690667\|gb\|GW783991.1\|GW783991_PBC064A05_08419_Parthenium_ |
| | argentatum_cold_acclimated_bark_cDNA_library_(PBC)_Parthenium_argentatum_ |
| | cDNA_clone_PBC064A05,_mRNA_sequence |
| 317 | gi\|294690572\|gb\|GW783978.1\|GW783978_PBC063G11_08406_Parthenium_ |
| | argentatum_cold_acclimated_bark_cDNA_library_(PBC)_Parthenium_argentatum_ |
| | cDNA_clone_PBC063G11,_mRNA_sequence |
| 318 | gi\|294690551\|gb\|GW783957.1\|GW783957_PBC063E11_08385_Parthenium_ |
| | argentatum_cold_acclimated_bark_cDNA_library_(PBC)_Parthenium_argentatum_ |
| | cDNA_clone_PBC063E11,_mRNA_sequence |
| 319 | gi\|294690385\|gb\|GW783873.1\|GW783873_PBC062F07_08301_Parthenium_ |
| | argentatum_cold_acclimated_bark_cDNA_library_(PBC)_Parthenium_argentatum_ |
| | cDNA_clone_PBC062F07,_mRNA_sequence |
| 320 | gi\|294690251\|gb\|GW783821.1\|GW783821_PBC062B05_08249_Parthenium_ |
| | argentatum_cold_acclimated_bark_cDNA_library_(PBC)_Parthenium_argentatum_ |
| | cDNA_clone_PBC062B05,_mRNA_sequence |
| 321 | gi\|294690207\|gb\|GW783777.1\|GW783777_PBC061E11_08205_Parthenium_ |
| | argentatum_cold_acclimated_bark_cDNA_library_(PBC)_Parthenium_argentatum_ |
| | cDNA_clone_PBC061E11,_mRNA_sequence |
| 322 | gi\|294690085\|gb\|GW783737.1\|GW783737_PBC061B05_08165_Parthenium_ |
| | argentatum_cold_acclimated_bark_cDNA_library_(PBC)_Parthenium_argentatum_ |
| | cDNA_clone_PBC061B05,_mRNA_sequence |
| 323 | gi\|294690070\|gb\|GW783722.1\|GW783722_PBC060H10_08150_Parthenium_ |
| | argentatum_cold_acclimated_bark_cDNA_library_(PBC)_Parthenium_argentatum_ |
| | cDNA_clone_PBC060H10,_mRNA_sequence |
| 324 | gi\|294690069\|gb\|GW783721.1\|GW783721_PBC060H09_08149_Parthenium_ |
| | argentatum_cold_acclimated_bark_cDNA_library_(PBC)_Parthenium_argentatum_ |
| | cDNA_clone_PBC060H09,_mRNA_sequence |
| 325 | gi\|294690060\|gb\|GW783712.1\|GW783712_PBC060G12_08140_Parthenium_ |
| | argentatum_cold_acclimated_bark_cDNA_library_(PBC)_Parthenium_argentatum_ |
| | cDNA_clone_PBC060G12,_mRNA_sequence |
| 326 | gi\|294690058\|gb\|GW783710.1\|GW783710_PBC060G10_08138_Parthenium_ |
| | argentatum_cold_acclimated_bark_cDNA_library_(PBC)_Parthenium_argentatum_ |
| | cDNA_clone_PBC060G10,_mRNA_sequence |
| 327 | gi\|294690034\|gb\|GW783686.1\|GW783686_PBC060E09_08114_Parthenium_ |
| | argentatum_cold_acclimated_bark_cDNA_library_(PBC)_Parthenium_argentatum_ |
| | cDNA_clone_PBC060E09,_mRNA_sequence |
| 328 | gi\|294690014\|gb\|GW776082.1\|GW776082_PAB907b09_00510_Parthenium_ |
| | argentatum_cold_acclimated_bark_cDNA_library_(PAB)_Parthenium_argentatum_ |
| | cDNA_clone_PAB067b09,_mRNA_sequence |
| 329 | gi\|294689982\|gb\|GW776050.1\|GW776050_PAB906h02_00478_Parthenium_ |
| | argentatum_cold_acclimated_bark_cDNA_library_(PAB)_Parthenium_argentatum_ |
| | cDNA_clone_PAB906h02,_mRNA_sequence |
| 330 | gi\|294689958\|gb\|GW776026.1\|GW776026_PAB906g07_08743_Parthenium_ |
| | argentatum_cold_acclimated_bark_cDNA_library_(PAB)_Parthenium_argentatum_ |
| | cDNA_clone_PAB906g07,_mRNA_sequence |
| 331 | gi\|294689839\|gb\|GW786731.1\|GW786731_PBC094D12_11159_Parthenium_ |
| | argentatum_cold_acclimated_bark_cDNA_library_(PBC)_Parthenium_argentatum_ |
| | cDNA_clone_PBC094D12,_mRNA_sequence |
| 332 | gi\|294689831\|gb\|GW786723.1\|GW786723_PBC094D05_11151_Parthenium_ |
| | argentatum_cold_acclimated_bark_cDNA_library_(PBC)_Parthenium_argentatum_ |
| | cDNA_clone_PBC094D05,_mRNA_sequence |
| 333 | gi\|294689826\|gb\|GW786718.1\|GW786718_PBC094C11_11146_Parthenium_ |
| | argentatum_cold_acclimated_bark_cDNA_library_(PBC)_Parthenium_argentatum_ |
| | cDNA_clone_PBC094C11,_mRNA_sequence |
| 334 | gi\|294689798\|gb\|GW786690.1\|GW786690_PBC094A06_11118_Parthenium_ |
| | argentatum_cold_acclimated_bark_cDNA_library_(PBC)_Parthenium_argentatum_ |
| | cDNA_clone_PBC094A06,_mRNA_sequence |
| 335 | gi\|294689751\|gb\|GW775946.1\|GW775946_PAB905c05_00374_Parthenium_ |
| | argentatum_cold_acclimated_bark_cDNA_library_(PAB)_Parthenium_argentatum_ |
| | cDNA_clone_PAB905c05,_mRNA_sequence |
| 336 | gi\|294689647\|gb\|GW786666.1\|GW786666_PBC093G05_11094_Parthenium_ |
| | argentatum_cold_acclimated_bark_cDNA_library_(PBC)_Parthenium_argentatum_ |
| | cDNA_clone_PBC093G05,_mRNA_sequence |
| 337 | gi\|294689589\|gb\|GW786608.1\|GW786608_PBC093B05_11036_Parthenium_ |
| | argentatum_cold_acclimated_bark_cDNA_library_(PBC)_Parthenium_argentatum_ |
| | cDNA_clone_PBC093B05,_mRNA_sequence |
| 338 | gi\|294689588\|gb\|GW786607.1\|GW786607_PBC093B05_11035_Parthenium_ |
| | argentatum_cold_acclimated_bark_cDNA_library_(PBC)_Parthenium_argentatum_ |
| | cDNA_clone_PBC093B05,_mRNA_sequence |
| 339 | gi\|294689541\|gb\|GW775895.1\|GW775895_PAB904b04_00323_Parthenium_ |
| | argentatum_cold_acclimated_bark_cDNA_library_(PAB)_Parthenium_argentatum_ |
| | cDNA_clone_PAB904b04,_mRNA_sequence |
| 340 | gi\|294689534\|gb\|GW775888.1\|GW775888_PAB904d03_00316_Parthenium_ |
| | argentatum_cold_acclimated_bark_cDNA_library_(PAB)_Parthenium_argentatum_ |
| | cDNA_clone_PAB904d03,_mRNA_sequence |
| 341 | gi\|294689361\|gb\|GW786539.1\|GW786539_PBC092D05_10967_Parthenium_ |
| | argentatum_cold_acclimated_bark_cDNA_library_(PBC)_Parthenium_argentatum_ |
| | cDNA_clone_PBC092D05,_mRNA_sequence |
| 342 | gi\|294689279\|gb\|GW775792.1\|GW775792_PAB903c06_00220_Parthenium_ |
| | argentatum_cold_acclimated_bark_cDNA_library_(PAB)_Parthenium_argentatum_ |
| | cDNA_clone_PAB903c06,_mRNA_sequence |
| 343 | gi\|294689277\|gb\|GW775790.1\|GW775790_PAB903c10_00218_Parthenium_ |
| | argentatum_cold_acclimated_bark_cDNA_library_(PAB)_Parthenium_argentatum_ |
| | cDNA_clone_PAB903c10,_mRNA_sequence |
| 344 | gi\|294689161\|gb\|GW786498.1\|GW786498_PBC091H05_10926_Parthenium_ |
| | argentatum_cold_acclimated_bark_cDNA_library_(PBC)_Parthenium_argentatum_ |
| | cDNA_clone_PBC091H05,_mRNA_sequence |
| 345 | gi\|294689146\|gb\|GW786483.1\|GW786483_PBC091G05_10911_Parthenium_ |
| | argentatum_cold_acclimated_bark_cDNA_library_(PBC)_Parthenium_argentatum_ |
| | cDNA_clone_PBC091G05,_mRNA_sequence |
| 346 | gi\|294689119\|gb\|GW786456.1\|GW786456_PBC091D11_10884_Parthenium_ |
| | argentatum_cold_acclimated_bark_cDNA_library_(PBC)_Parthenium_argentatum_ |
| | cDNA_clone_PBC091D11,_mRNA_sequence |
| 347 | gi\|294689112\|gb\|GW786449.1\|GW786449_PBC091D05_10877_Parthenium_ |
| | argentatum_cold_acclimated_bark_cDNA_library_(PBC)_Parthenium_argentatum_ |
| | cDNA_clone_PBC091D05,_mRNA_sequence |
| 348 | gi\|294689105\|gb\|GW786442.1\|GW786442_PBC091C09_10870_Parthenium_ |
| | argentatum_cold_acclimated_bark_cDNA_library_(PBC)_Parthenium_argentatum_ |
| | cDNA_clone_PBC091C09,_mRNA_sequence |
| 349 | gi\|294689080\|gb\|GW775752.1\|GW775752_PAB903b09_00180_Parthenium_ |
| | argentatum_cold_acclimated_bark_cDNA_library_(PAB)_Parthenium_argentatum_ |
| | cDNA_clone_PAB903b09,_mRNA_sequence |
| 350 | gi\|294689039\|gb\|GW775711.1\|GW775711_PAB902e03_00139_Parthenium_ |
| | argentatum_cold_acclimated_bark_cDNA_library_(PAB)_Parthenium_argentatum_ |
| | cDNA_clone_PAB902e03,_mRNA_sequence |
| 351 | gi\|294689036\|gb\|GW775708.1\|GW775708_PAB902h05_00136_Parthenium_ |
| | argentatum_cold_acclimated_bark_cDNA_library_(PAB)_Parthenium_argentatum_ |
| | cDNA_clone_PAB902h05,_mRNA_sequence |
| 352 | gi\|294689028\|gb\|GW775700.1\|GW775700_PAB902c02_00128_Parthenium_ |
| | argentatum_cold_acclimated_bark_cDNA_library_(PAB)_Parthenium_argentatum_ |
| | cDNA_clone_PAB902c02,_mRNA_sequence |
| 353 | gi\|294689025\|gb\|GW775697.1\|GW775697_PAB902c11_00125_Parthenium_ |
| | argentatum_cold_acclimated_bark_cDNA_library_(PAB)_Parthenium_argentatum_ |
| | cDNA_clone_PAB902c11,_mRNA_sequence |
| 354 | gi\|294689020\|gb\|GW775692.1\|GW775692_PAB902h08_00120_Parthenium_ |
| | argentatum_cold_acclimated_bark_cDNA_library_(PAB)_Parthenium_argentatum_ |
| | cDNA_clone_PAB902h08,_mRNA_sequence |
| 355 | gi\|294688934\|gb\|GW786430.1\|GW786430_PBC091B09_10858_Parthenium_ |
| | argentatum_cold_acclimated_bark_cDNA_library_(PAB)_Parthenium_argentatum_ |
| | cDNA_clone_PBC091B09,_mRNA_sequence |
| 356 | gi\|294688912\|gb\|GW786408.1\|GW786408_PBC090H07_10836_Parthenium_ |
| | argentatum_cold_acclimated_bark_cDNA_library_(PAB)_Parthenium_argentatum_ |
| | cDNA_clone_PBC090H07,_mRNA_sequence |
| 357 | gi\|294688882\|gb\|GW786378.1\|GW786378_PBC090E06_10806_Parthenium_ |
| | argentatum_cold_acclimated_bark_cDNA_library_(PAB)_Parthenium_argentatum_ |
| | cDNA_clone_PBC090E06,_mRNA_sequence |
| 358 | gi\|294688873\|gb\|GW786369.1\|GW786369_PBC090D09_10797_Parthenium_ |
| | argentatum_cold_acclimated_bark_cDNA_library_(PAB)_Parthenium_argentatum_ |
| | cDNA_clone_PBC090D09,_mRNA_sequence |
| 359 | gi\|294688866\|gb\|GW786362.1\|GW786362_PBC090D05_10790_Parthenium_ |
| | argentatum_cold_acclimated_bark_cDNA_library_(PAB)_Parthenium_argentatum_ |
| | cDNA_clone_PBC090D05,_mRNA_sequence |
| 360 | gi\|294688865\|gb\|GW786361.1\|GW786361_PBC090C12_10789_Parthenium_ |
| | argentatum_cold_acclimated_bark_cDNA_library_(PAB)_Parthenium_argentatum_ |
| | cDNA_clone_PBC090C12,_mRNA_sequence |
| 361 | gi\|294688852\|gb\|GW775683.1\|GW775683_PAB902c05_00111_Parthenium_ |
| | argentatum_cold_acclimated_bark_cDNA_library_(PAB)_Parthenium_argentatum_ |
| | cDNA_clone_PAB902c05,_mRNA_sequence |
| 362 | gi\|294688846\|gb\|GW775677.1\|GW775677_PAB902e04_00105_Parthenium_ |
| | argentatum_cold_acclimated_bark_cDNA_library_(PAB)_Parthenium_argentatum_ |
| | cDNA_clone_PAB902e04,_mRNA_sequence |
| 363 | gi\|294688701\|gb\|GW786356.1\|GW786356_PBC090C07_10784_Parthenium_ |
| | argentatum_cold_acclimated_bark_cDNA_library_(PAB)_Parthenium_argentatum_ |
| | cDNA_clone_PBC090C07,_mRNA_sequence |
| 364 | gi\|294688701\|gb\|GW786356.1\|GW786356_PBC090C07_10784_Parthenium_ |
| | argentatum_cold_acclimated_bark_cDNA_library_(PAB)_Parthenium_argentatum_ |
| | cDNA_clone_PBC090C07,_mRNA_sequence |
| 365 | gi\|294688651\|gb\|GW786306.1\|GW786306_PBC089F05_10734_Parthenium_ |
| | argentatum_cold_acclimated_bark_cDNA_library_(PAB)_Parthenium_argentatum_ |
| | cDNA_clone_PBC089F05,_mRNA_sequence |
| 366 | gi\|294688635\|gb\|GW786290.1\|GW786290_PBC089D12_10718_Parthenium_ |
| | argentatum_cold_acclimated_bark_cDNA_library_(PAB)_Parthenium_argentatum_ |
| | cDNA_clone_PBC089D12,_mRNA_sequence |
| 367 | gi\|294688618\|gb\|GW775609.1\|GW775609_PAB901d06_00037_Parthenium_ |
| | argentatum_cold_acclimated_bark_cDNA_library_(PAB)_Parthenium_argentatum_ |
| | cDNA_clone_PAB901d06,_mRNA_sequence |
| 368 | gi\|294688613\|gb\|GW775604.1\|GW775604_PAB901c03_00032_Parthenium_ |
| | argentatum_cold_acclimated_bark_cDNA_library_(PAB)_Parthenium_argentatum_ |
| | cDNA_clone_PAB901c03,_mRNA_sequence |
| 369 | gi\|294688601\|gb\|GW775592.1\|GW775592_PAB901a11_00020_Parthenium_ |
| | argentatum_cold_acclimated_bark_cDNA_library_(PAB)_Parthenium_argentatum_ |
| | cDNA_clone_PAB901a11,_mRNA_sequence |
| 370 | gi\|294688592\|gb\|GW775583.1\|GW775583_PAB901e05_00011_Parthenium_ |
| | argentatum_cold_acclimated_bark_cDNA_library_(PAB)_Parthenium_argentatum_ |
| | cDNA_clone_PAB901e05,_mRNA_sequence |
| 371 | gi\|294688500\|gb\|GW786276.1\|GW786276_PBC089C09_10704_Parthenium_ |
| | argentatum_cold_acclimated_bark_cDNA_library_(PAB)_Parthenium_argentatum_ |
| | cDNA_clone_PBC089C09,_mRNA_sequence |
| 372 | gi\|294688478\|gb\|GW786254.1\|GW786254_PBC089A11_10682_Parthenium_ |
| | argentatum_cold_acclimated_bark_cDNA_library_(PAB)_Parthenium_argentatum_ |
| | cDNA_clone_PBC089A11,_mRNA_sequence |
| 373 | gi\|294688459\|gb\|GW786235.1\|GW786235_PBC088H05_10663_Parthenium_ |
| | argentatum_cold_acclimated_bark_cDNA_library_(PAB)_Parthenium_argentatum_ |
| | cDNA_clone_PBC088H05,_mRNA_sequence |
| 374 | gi\|294688450\|gb\|GW786226.1\|GW786226_PBC088G07_10654_Parthenium_ |
| | argentatum_cold_acclimated_bark_cDNA_library_(PAB)_Parthenium_argentatum_ |
| | cDNA_clone_PBC088G07,_mRNA_sequence |
| 375 | gi\|294688446\|gb\|GW786222.1\|GW786222_PBC088G05_10650_Parthenium_ |
| | argentatum_cold_acclimated_bark_cDNA_library_(PAB)_Parthenium_argentatum_ |
| | cDNA_clone_PBC088G05,_mRNA_sequence |
| 376 | gi\|294688432\|gb\|GW786208.1\|GW786208_PBC088F05_10636_Parthenium_ |
| | argentatum_cold_acclimated_bark_cDNA_library_(PAB)_Parthenium_argentatum_ |
| | cDNA_clone_PBC088F05,_mRNA_sequence |
| 377 | gi\|294688425\|gb\|GW786201.1\|GW786201_PBC088E05_10629_Parthenium_ |
| | argentatum_cold_acclimated_bark_cDNA_library_(PAB)_Parthenium_argentatum_ |
| | cDNA_clone_PBC088E05,_mRNA_sequence |
| 378 | gi\|294688270\|gb\|GW786126.1\|GW786126_PBC087F09_10554_Parthenium_ |
| | argentatum_cold_acclimated_bark_cDNA_library_(PAB)_Parthenium_argentatum_ |
| | cDNA_clone_PBC087F09,_mRNA_sequence |
| 379 | gi\|294688173\|gb\|GW778606.1\|GW778606_PBC004A11_03034_Parthenium_ |
| | argentatum_cold_acclimated_bark_cDNA_library_(PAB)_Parthenium_argentatum_ |
| | cDNA_clone_PBC004A11,_mRNA_sequence |
| 380 | gi\|294688144\|gb\|GW778577.1\|GW778577_PBC003G05_03005_Parthenium_ |
| | argentatum_cold_acclimated_bark_cDNA_library_(PAB)_Parthenium_argentatum_ |
| | cDNA_clone_PBC003G05,_mRNA_sequence |
| 381 | gi\|294688119\|gb\|GW778552.1\|GW778552_PBC003E04_02980_Parthenium_ |
| | argentatum_cold_acclimated_bark_cDNA_library_(PAB)_Parthenium_argentatum_ |
| | cDNA_clone_PBC003E04,_mRNA_sequence |
| 382 | gi\|294688103\|gb\|GW778536.1\|GW778536_PBC003C11_02964_Parthenium_ |
| | argentatum_cold_acclimated_bark_cDNA_library_(PAB)_Parthenium_argentatum_ |
| | cDNA_clone_PBC003C11,_mRNA_sequence |
| 383 | gi\|294688020\|gb\|GW778535.1\|GW778535_PBC003C10_02963_Parthenium_ |
| | argentatum_cold_acclimated_bark_cDNA_library_(PAB)_Parthenium_argentatum_ |
| | cDNA_clone_PBC003C10,_mRNA_sequence |
| 384 | gi\|294688020\|gb\|GW778535.1\|GW778535_PBC003C10_02963_Parthenium_ |
| | argentatum_cold_acclimated_bark_cDNA_library_(PAB)_Parthenium_argentatum_ |
| | cDNA_clone_PBC003C10,_mRNA_sequence |
| 385 | gi\|294687992\|gb\|GW778507.1\|GW778507_PBC003A05_02935_Parthenium_ |
| | argentatum_cold_acclimated_bark_cDNA_library_(PAB)_Parthenium_argentatum_ |
| | cDNA_clone_PBC003A05,_mRNA_sequence |
| 386 | gi\|294687832\|gb\|GW778429.1\|GW778429_PBC002B04_02857_Parthenium_ |
| | argentatum_cold_acclimated_bark_cDNA_library_(PAB)_Parthenium_argentatum_ |
| | cDNA_clone_PBC002B04,_mRNA_sequence |
| 387 | gi\|294687815\|gb\|GW778412.1\|GW778412_PBC001H11_02840_Parthenium_ |
| | argentatum_cold_acclimated_bark_cDNA_library_(PAB)_Parthenium_argentatum_ |
| | cDNA_clone_PBC001H11,_mRNA_sequence |
| 388 | gi\|294687684\|gb\|GW778352.1\|GW778352_PBC001C08_02780_Parthenium_ |
| | argentatum_cold_acclimated_bark_cDNA_library_(PAB)_Parthenium_argentatum_ |
| | cDNA_clone_PBC001C08,_mRNA_sequence |
| 389 | gi\|294687676\|gb\|GW778344.1\|GW778344_PBC001B11_02772_Parthenium_ |
| | argentatum_cold_acclimated_bark_cDNA_library_(PAB)_Parthenium_argentatum_ |
| | cDNA_clone_PBC001B11,_mRNA_sequence |
| 390 | gi\|294687656\|gb\|GW778324.1\|GW778324_PBC001A02_02752_Parthenium_ |
| | argentatum_cold_acclimated_bark_cDNA_library_(PAB)_Parthenium_argentatum_ |
| | cDNA_clone_PBC001A02,_mRNA_sequence |
| 391 | gi\|294687643\|gb\|GW778311.1\|GW778311_PAB936D11_02739_Parthenium_ |
| | argentatum_cold_acclimated_bark_cDNA_library_(PAB)_Parthenium_argentatum_ |
| | cDNA_clone_PAB936D11,_mRNA_sequence |
| 392 | gi\|294687642\|gb\|GW778310.1\|GW778310_PAB936C04_02738_Parthenium_ |
| | argentatum_cold_acclimated_bark_cDNA_library_(PAB)_Parthenium_argentatum_ |
| | cDNA_clone_PAB936C04,_mRNA_sequence |
| 393 | gi\|294687641\|gb\|GW778309.1\|GW778309_PAB936F01_02737_Parthenium_ |
| | argentatum_cold_acclimated_bark_cDNA_library_(PAB)_Parthenium_argentatum_ |
| | cDNA_clone_PAB936F01,_mRNA_sequence |
| 394 | gi\|294687637\|gb\|GW778305.1\|GW778305_PAB936B01_02733_Parthenium_ |
| | argentatum_cold_acclimated_bark_cDNA_library_(PAB)_Parthenium_argentatum_ |
| | cDNA_clone_PAB936B01,_mRNA_sequence |
| 395 | gi\|294687609\|gb\|GW778277.1\|GW778277_PAB936A10_02705_Parthenium_ |
| | argentatum_cold_acclimated_bark_cDNA_library_(PAB)_Parthenium_argentatum_ |
| | cDNA_clone_PAB936A10,_mRNA_sequence |
| 396 | gi\|294687593\|gb\|GW778261.1\|GW778261_PAB936D01_02689_Parthenium_ |
| | argentatum_cold_acclimated_bark_cDNA_library_(PAB)_Parthenium_argentatum_ |
| | cDNA_clone_PAB936D01,_mRNA_sequence |
| 397 | gi\|294687588\|gb\|GW778256.1\|GW778256_PAB936D10_02684_Parthenium_ |
| | argentatum_cold_acclimated_bark_cDNA_library_(PAB)_Parthenium_argentatum_ |
| | cDNA_clone_PAB936D10,_mRNA_sequence |
| 398 | gi\|294687528\|gb\|GW778196.1\|GW778196_PAB935H10_02624_Parthenium_ |
| | argentatum_cold_acclimated_bark_cDNA_library_(PAB)_Parthenium_argentatum_ |
| | cDNA_clone_PAB935H10,_mRNA_sequence |
| 399 | gi\|294687480\|gb\|GW778148.1\|GW778148_PAB935F10_02576_Parthenium_ |
| | argentatum_cold_acclimated_bark_cDNA_library_(PAB)_Parthenium_argentatum_ |
| | cDNA_clone_PAB935F10,_mRNA_sequence |
| 400 | gi\|294687425\|gb\|GW778093.1\|GW778093_PAB934G04_02521_Parthenium_ |
| | argentatum_cold_acclimated_bark_cDNA_library_(PAB)_Parthenium_argentatum_ |
| | cDNA_clone_PAB934G04,_mRNA_sequence |
| 401 | gi\|294687387\|gb\|GW778055.1\|GW778055_PAB933B05_02483_Parthenium_ |
| | argentatum_cold_acclimated_bark_cDNA_library_(PAB)_Parthenium_argentatum_ |
| | cDNA_clone_PAB933B05,_mRNA_sequence |
| 402 | gi\|294687380\|gb\|GW778048.1\|GW778048_PAB933D04_02476_Parthenium_ |
| | argentatum_cold_acclimated_bark_cDNA_library_(PAB)_Parthenium_argentatum_ |
| | cDNA_clone_PAB933D04,_mRNA_sequence |
| 403 | gi\|294687364\|gb\|GW778032.1\|GW778032_PAB933H11_02460_Parthenium_ |
| | argentatum_cold_acclimated_bark_cDNA_library_(PAB)_Parthenium_argentatum_ |
| | cDNA_clone_PAB933H11,_mRNA_sequence |
| 404 | gi\|294687346\|gb\|GW778014.1\|GW778014_PAB933E12_02442_Parthenium_ |
| | argentatum_cold_acclimated_bark_cDNA_library_(PAB)_Parthenium_argentatum_ |
| | cDNA_clone_PAB933E12,_mRNA_sequence |
| 405 | gi\|294686313\|gb\|GW781110.1\|GW781110_PBC031F03_05538_Parthenium_ |
| | argentatum_cold_acclimated_bark_cDNA_library_(PAB)_Parthenium_argentatum_ |
| | cDNA_clone_PBC031F03,_mRNA_sequence |
| 406 | gi\|294686307\|gb\|GW781104.1\|GW781104_PBC031E08_05532_Parthenium_ |
| | argentatum_cold_acclimated_bark_cDNA_library_(PAB)_Parthenium_argentatum_ |
| | cDNA_clone_PBC031E08,_mRNA_sequence |
| 407 | gi\|294686290\|gb\|GW781087.1\|GW781087_PBC031D03_05515_Parthenium_ |
| | argentatum_cold_acclimated_bark_cDNA_library_(PAB)_Parthenium_argentatum_ |
| | cDNA_clone_PBC031D03,_mRNA_sequence |
| 408 | gi\|294686101\|gb\|GW781062.1\|GW781062_PBC031A11_05490_Parthenium_ |
| | argentatum_cold_acclimated_bark_cDNA_library_(PAB)_Parthenium_argentatum_ |
| | cDNA_clone_PBC031A11,_mRNA_sequence |
| 409 | gi\|294685811\|gb\|GW780936.1\|GW780936_PBC029F05_05364_Parthenium_ |
| | argentatum_cold_acclimated_bark_cDNA_library_(PAB)_Parthenium_argentatum_ |
| | cDNA_clone_PBC029F05,_mRNA_sequence |
| 410 | gi\|294685796\|gb\|GW780921.1\|GW780921_PBC029E02_05349_Parthenium_ |
| | argentatum_cold_acclimated_bark_cDNA_library_(PAB)_Parthenium_argentatum_ |
| | cDNA_clone_PBC029E02,_mRNA_sequence |
| 411 | gi\|294685789\|gb\|GW780914.1\|GW780914_PBC029D07_05342_Parthenium_ |
| | argentatum_cold_acclimated_bark_cDNA_library_(PAB)_Parthenium_argentatum_ |
| | cDNA_clone_PBC029D07,_mRNA_sequence |
| 412 | gi\|294685610\|gb\|GW780894.1\|GW780894_PBC029B11_05322_Parthenium_ |
| | argentatum_cold_acclimated_bark_cDNA_library_(PAB)_Parthenium_argentatum_ |
| | cDNA_clone_PBC029B11,_mRNA_sequence |
| 413 | gi\|294685600\|gb\|GW780884.1\|GW780884_PBC029A12_05312_Parthenium_ |
| | argentatum_cold_acclimated_bark_cDNA_library_(PAB)_Parthenium_argentatum_ |
| | cDNA_clone_PBC029A12,_mRNA_sequence |
| 414 | gi\|294685567\|gb\|GW780851.1\|GW780851_PBC028G03_05279_Parthenium_ |
| | argentatum_cold_acclimated_bark_cDNA_library_(PAB)_Parthenium_argentatum_ |
| | cDNA_clone_PBC028G03,_mRNA_sequence |
| 415 | gi\|294685550\|gb\|GW780834.1\|GW780834_PBC028E05_05262_Parthenium_ |
| | argentatum_cold_acclimated_bark_cDNA_library_(PAB)_Parthenium_argentatum_ |
| | cDNA_clone_PBC028E05,_mRNA_sequence |
| 416 | gi\|294685378\|gb\|GW780826.1\|GW780826_PBC028D09_05254_Parthenium_ |
| | argentatum_cold_acclimated_bark_cDNA_library_(PAB)_Parthenium_argentatum_ |
| | cDNA_clone_PBC028D09,_mRNA_sequence |
| 417 | gi\|294685373\|gb\|GW780821.1\|GW780821_PBC028D04_05249_Parthenium_ |
| | argentatum_cold_acclimated_bark_cDNA_library_(PAB)_Parthenium_argentatum_ |
| | cDNA_clone_PBC028D04,_mRNA_sequence |
| 418 | gi\|294685341\|gb\|GW780789.1\|GW780789_PBC028A07_05217_Parthenium_ |
| | argentatum_cold_acclimated_bark_cDNA_library_(PAB)_Parthenium_argentatum_ |
| | cDNA_clone_PBC028A07,_mRNA_sequence |
| 419 | gi\|294685317\|gb\|GW780765.1\|GW780765_PBC027G05_05193_Parthenium_ |
| | argentatum_cold_acclimated_bark_cDNA_library_(PAB)_Parthenium_argentatum_ |
| | cDNA_clone_PBC027G05,_mRNA_sequence |
| 420 | gi\|294685316\|gb\|GW780764.1\|GW780764_PBC027G04_05192_Parthenium_ |
| | argentatum_cold_acclimated_bark_cDNA_library_(PAB)_Parthenium_argentatum_ |
| | cDNA_clone_PBC027G04,_mRNA_sequence |
| 421 | gi\|294685311\|gb\|GW780759.1\|GW780759_PBC027F11_05187_Parthenium_ |
| | argentatum_cold_acclimated_bark_cDNA_library_(PAB)_Parthenium_argentatum_ |
| | cDNA_clone_PBC027F11,_mRNA_sequence |
| 422 | gi\|294684877\|gb\|GW780653.1\|GW780653_PBC026E06_05081_Parthenium_ |
| | argentatum_cold_acclimated_bark_cDNA_library_(PAB)_Parthenium_argentatum_ |
| | cDNA_clone_PBC026E06,_mRNA_sequence |
| 423 | gi\|294684874\|gb\|GW780650.1\|GW780650_PBC026E02_05078_Parthenium_ |
| | argentatum_cold_acclimated_bark_cDNA_library_(PAB)_Parthenium_argentatum_ |
| | cDNA_clone_PBC026E02,_mRNA_sequence |
| 424 | gi\|294684864\|gb\|GW780640.1\|GW780640_PBC026D03_05068_Parthenium_ |
| | argentatum_cold_acclimated_bark_cDNA_library_(PAB)_Parthenium_argentatum_ |
| | cDNA_clone_PBC026D03,_mRNA_sequence |
| 425 | gi\|294684855\|gb\|GW780631.1\|GW780631_PBC026C06_05059_Parthenium_ |
| | argentatum_cold_acclimated_bark_cDNA_library_(PAB)_Parthenium_argentatum_ |
| | cDNA_clone_PBC026C06,_mRNA_sequence |
| 426 | gi\|294684818\|gb\|GW780594.1\|GW780594_PBC025H02_05022_Parthenium_ |
| | argentatum_cold_acclimated_bark_cDNA_library_(PAB)_Parthenium_argentatum_ |
| | cDNA_clone_PBC025H02,_mRNA_sequence |
| 427 | gi\|294684644\|gb\|GW780584.1\|GW780584_PBC025G03_05012_Parthenium_ |
| | argentatum_cold_acclimated_bark_cDNA_library_(PAB)_Parthenium_argentatum_ |
| | cDNA_clone_PBC025G03,_mRNA_sequence |
| 428 | gi\|294684629\|gb\|GW780569.1\|GW780569_PBC025E10_04997_Parthenium_ |
| | argentatum_cold_acclimated_bark_cDNA_library_(PAB)_Parthenium_argentatum_ |
| | cDNA_clone_PBC025E10,_mRNA_sequence |
| 429 | gi\|294684610\|gb\|GW780550.1\|GW780550_PBC025D02_04978_Parthenium_ |
| | argentatum_cold_acclimated_bark_cDNA_library_(PAB)_Parthenium_argentatum_ |
| | cDNA_clone_PBC025D02,_mRNA_sequence |
| 430 | gi\|294684572\|gb\|GW780512.1\|GW780512_PBC024H08_04940_Parthenium_ |
| | argentatum_cold_acclimated_bark_cDNA_library_(PAB)_Parthenium_argentatum_ |
| | cDNA_clone_PBC024H08,_mRNA_sequence |
| 431 | gi\|294682355\|gb\|GW783666.1\|GW783666_PBC060C12_08094_Parthenium_ |
| | argentatum_cold_acclimated_bark_cDNA_library_(PAB)_Parthenium_argentatum_ |
| | cDNA_clone_PBC060C12,_mRNA_sequence |
| 432 | gi\|294682350\|gb\|GW783661.1\|GW783661_PBC060C06_08089_Parthenium_ |
| | argentatum_cold_acclimated_bark_cDNA_library_(PAB)_Parthenium_argentatum_ |
| | cDNA_clone_PBC060C06,_mRNA_sequence |
| 433 | gi\|294682348\|gb\|GW783639.1\|GW783659_PBC060C05_08087_Parthenium_ |
| | argentatum_cold_acclimated_bark_cDNA_library_(PAB)_Parthenium_argentatum_ |
| | cDNA_clone_PBC060C05,_mRNA_sequence |
| 434 | gi\|294682308\|gb\|GW783619.1\|GW783619_PBC059G11_08047_Parthenium_ |
| | argentatum_cold_acclimated_bark_cDNA_library_(PAB)_Parthenium_argentatum_ |
| | cDNA_clone_PBC059G11,_mRNA_sequence |
| 435 | gi\|294682283\|gb\|GW783594.1\|GW783594_PBC059E10_08022_Parthenium_ |
| | argentatum_cold_acclimated_bark_cDNA_library_(PAB)_Parthenium_argentatum_ |
| | cDNA_clone_PBC059E10,_mRNA_sequence |
| 436 | gi\|294682255\|gb\|GW777957.1\|GW777957_PAB928D03_02385_Parthenium_ |
| | argentatum_cold_acclimated_bark_cDNA_library_(PAB)_Parthenium_argentatum_ |
| | cDNA_clone_PAB928D03,_mRNA_sequence |
| 437 | gi\|294682255\|gb\|GW777957.1\|GW777957_PAB928D03_02385_Parthenium_ |
| | argentatum_cold_acclimated_bark_cDNA_library_(PAB)_Parthenium_argentatum_ |
| | cDNA_clone_PAB928D03,_mRNA_sequence |
| 438 | gi\|294682242\|gb\|GW777944.1\|GW777944_PAB928E09_02372_Parthenium_ |
| | argentatum_cold_acclimated_bark_cDNA_library_(PAB)_Parthenium_argentatum_ |
| | cDNA_clone_PAB928E09,_mRNA_sequence |
| 439 | gi\|294682210\|gb\|GW777912.1\|GW777912_PAB928B08_02340_Parthenium_ |
| | argentatum_cold_acclimated_bark_cDNA_library_(PAB)_Parthenium_argentatum_ |
| | cDNA_clone_PAB928B08,_mRNA_sequence |
| 440 | gi\|294682207\|gb\|GW777909.1\|GW777909_PAB928B12_02337_Parthenium_ |
| | argentatum_cold_acclimated_bark_cDNA_library_(PAB)_Parthenium_argentatum_ |
| | cDNA_clone_PAB928B12,_mRNA_sequence |
| 441 | gi\|294682206\|gb\|GW777908.1\|GW777908_PAB928H07_02336_Parthenium_ |
| | argentatum_cold_acclimated_bark_cDNA_library_(PAB)_Parthenium_argentatum_ |
| | cDNA_clone_PAB928H07,_mRNA_sequence |
| 442 | gi\|294682202\|gb\|GW777904.1\|GW777904_PAB928G04_02332_Parthenium_ |
| | argentatum_cold_acclimated_bark_cDNA_library_(PAB)_Parthenium_argentatum_ |
| | cDNA_clone_PAB928H07,_mRNA_sequence |
| 443 | gi\|294682201\|gb\|GW783591.1\|GW783591_PBC059E7_08019_Parthenium_ |
| | argentatum_cold_acclimated_bark_cDNA_library_(PBC)_Parthenium_argentatum_ |
| | cDNA_clone_PBC059E7,_mRNA_sequence |
| 444 | gi\|294682193\|gb\|GW783583.1\|GW783583_PBC059D11_08011_Parthenium_ |
| | argentatum_cold_acclimated_bark_cDNA_library_(PBC)_Parthenium_argentatum_ |
| | cDNA_clone_PBC059D11,_mRNA_sequence |
| 445 | gi\|294682146\|gb\|GW783536.1\|GW783536_PBC058H08_07964_Parthenium_ |
| | argentatum_cold_acclimated_bark_cDNA_library_(PBC)_Parthenium_argentatum_ |
| | cDNA_clone_PBC058H08,_mRNA_sequence |
| 446 | gi\|294682119\|gb\|GW777900.1\|GW777900_PAB928F01_02328_Parthenium_ |
| | argentatum_cold_acclimated_bark_cDNA_library_(PAB)_Parthenium_argentatum_ |
| | cDNA_clone_PAB928F01,_mRNA_sequence |
| 447 | gi\|294682078\|gb\|GW777859.1\|GW777859_PAB927A11_02287_Parthenium_ |
| | argentatum_cold_acclimated_bark_cDNA_library_(PAB)_Parthenium_argentatum_ |
| | cDNA_clone_PAB927A11,_mRNA_sequence |
| 448 | gi\|294681980\|gb\|GW783475.1\|GW783475_PBC058C05_07903_Parthenium_ |
| | argentatum_cold_acclimated_bark_cDNA_library_(PBC)_Parthenium_argentatum_ |
| | cDNA_clone_PBC058C05,_mRNA_sequence |
| 449 | gi\|294681885\|gb\|GW777771.1\|GW777771_PAB926F01_02199_Parthenium_ |
| | argentatum_cold_acclimated_bark_cDNA_library_(PAB)_Parthenium_argentatum_ |
| | cDNA_clone_PAB926F01,_mRNA_sequence |
| 450 | gi\|294681867\|gb\|GW777753.1\|GW777753_PAB926C02_02181_Parthenium_ |
| | argentatum_cold_acclimated_bark_cDNA_library_(PAB)_Parthenium_argentatum_ |
| | cDNA_clone_PAB926C02,_mRNA_sequence |
| 451 | gi\|294681865\|gb\|GW777751.1\|GW777751_PAB926F04_02179_Parthenium_ |
| | argentatum_cold_acclimated_bark_cDNA_library_(PAB)_Parthenium_argentatum_ |
| | cDNA_clone_PAB926F04,_mRNA_sequence |
| 452 | gi\|294681827\|gb\|GW783401.1\|GW783401_PBC057D07_07829_Parthenium_ |
| | argentatum_cold_acclimated_bark_cDNA_library_(PBC)_Parthenium_argentatum_ |
| | cDNA_clone_PBC057D07,_mRNA_sequence |
| 453 | gi\|294681819\|gb\|GW783393.1\|GW783393_PBC057C11_07821_Parthenium_ |
| | argentatum_cold_acclimated_bark_cDNA_library_(PBC)_Parthenium_argentatum_ |
| | cDNA_clone_PBC057C11,_mRNA_sequence |
| 454 | gi\|294681804\|gb\|GW783378.1\|GW783378_PBC057B06_07806_Parthenium_ |
| | argentatum_cold_acclimated_bark_cDNA_library_(PBC)_Parthenium_argentatum_ |
| | cDNA_clone_PBC057B06,_mRNA_sequence |
| 455 | gi\|294681793\|gb\|GW783367.1\|GW783367_PBC057A07_07795_Parthenium_ |
| | argentatum_cold_acclimated_bark_cDNA_library_(PBC)_Parthenium_argentatum_ |
| | cDNA_clone_PBC057A07,_mRNA_sequence |
| 456 | gi\|294681793\|gb\|GW783367.1\|GW783367_PBC057A07_07795_Parthenium_ |
| | argentatum_cold_acclimated_bark_cDNA_library_(PBC)_Parthenium_argentatum_ |
| | cDNA_clone_PBC057A07,_mRNA_sequence |
| 457 | gi\|294681771\|gb\|GW777710.1\|GW777710_PAB926E02_02138_Parthenium_ |
| | argentatum_cold_acclimated_bark_cDNA_library_(PAB)_Parthenium_argentatum_ |
| | cDNA_clone_PAB926E02,_mRNA_sequence |
| 458 | gi\|294681755\|gb\|GW777694.1\|GW777694_PAB925E08_02122_Parthenium_ |
| | argentatum_cold_acclimated_bark_cDNA_library_(PAB)_Parthenium_argentatum_ |
| | cDNA_clone_PAB925E08,_mRNA_sequence |
| 459 | gi\|294681754\|gb\|GW777693.1\|GW777693_PAB925H05_02121_Parthenium_ |
| | argentatum_cold_acclimated_bark_cDNA_library_(PAB)_Parthenium_argentatum_ |
| | cDNA_clone_PAB925H05,_mRNA_sequence |
| 460 | gi\|294681732\|gb\|GW777671.1\|GW777671_PAB925A10_02099_Parthenium_ |
| | argentatum_cold_acclimated_bark_cDNA_library_(PAB)_Parthenium_argentatum_ |
| | cDNA_clone_PAB925A10,_mRNA_sequence |
| 461 | gi\|294681689\|gb\|GW783316.1\|GW783316_PBC056E05_07744_Parthenium_ |
| | argentatum_cold_acclimated_bark_cDNA_library_(PBC)_Parthenium_argentatum_ |
| | cDNA_clone_PBC056E05,_mRNA_sequence |
| 462 | gi\|294681679\|gb\|GW783306.1\|GW783306_PBC056C12_07734_Parthenium_ |
| | argentatum_cold_acclimated_bark_cDNA_library_(PBC)_Parthenium_argentatum_ |
| | cDNA_clone_PBC056C12,_mRNA_sequence |
| 463 | gi\|294681675\|gb\|GW783302.1\|GW783302_PBC056C08_07730_Parthenium_ |
| | argentatum_cold_acclimated_bark_cDNA_library_(PBC)_Parthenium_argentatum_ |
| | cDNA_clone_PBC056C08,_mRNA_sequence |
| 464 | gi\|294681672\|gb\|GW783299.1\|GW783299_PBC056C05_07727_Parthenium_ |
| | argentatum_cold_acclimated_bark_cDNA_library_(PBC)_Parthenium_argentatum_ |
| | cDNA_clone_PBC056C05,_mRNA_sequence |
| 465 | gi\|294681617\|gb\|GW777635.1\|GW777635_PAB925A11_02063_Parthenium_ |
| | argentatum_cold_acclimated_bark_cDNA_library_(PAB)_Parthenium_argentatum_ |
| | cDNA_clone_PAB925A11,_mRNA_sequence |
| 466 | gi\|294681599\|gb\|GW777617.1\|GW777617_PAB925D02_02045_Parthenium_ |
| | argentatum_cold_acclimated_bark_cDNA_library_(PAB)_Parthenium_argentatum_ |
| | cDNA_clone_PAB925D02,_mRNA_sequence |
| 467 | gi\|294681573\|gb\|GW777591.1\|GW777591_PAB924D01_02019_Parthenium_ |
| | argentatum_cold_acclimated_bark_cDNA_library_(PAB)_Parthenium_argentatum_ |
| | cDNA_clone_PAB924D01,_mRNA_sequence |
| 468 | gi\|294681573\|gb\|GW777591.1\|GW777591_PAB924D01_02019_Parthenium_ |
| | argentatum_cold_acclimated_bark_cDNA_library_(PAB)_Parthenium_argentatum_ |
| | cDNA_clone_PAB924D01,_mRNA_sequence |
| 469 | gi\|294681516\|gb\|GW783222.1\|GW783222_PBC055B08_07650_Parthenium_ |
| | argentatum_cold_acclimated_bark_cDNA_library_(PBC)_Parthenium_argentatum_ |
| | cDNA_clone_PBC055B08,_mRNA_sequence |
| 470 | gi\|294681491\|gb\|GW783197.1\|GW783197_PBC054G11_07625_Parthenium_ |
| | argentatum_cold_acclimated_bark_cDNA_library_(PBC)_Parthenium_argentatum_ |
| | cDNA_clone_PBC054G11,_mRNA_sequence |
| 471 | gi\|294681474\|gb\|GW777571.1\|GW777571_PAB924A11_01999_Parthenium_ |
| | argentatum_cold_acclimated_bark_cDNA_library_(PAB)_Parthenium_argentatum_ |
| | cDNA_clone_PAB924A11,_mRNA_sequence |
| 472 | gi\|294681452\|gb\|GW777549.1\|GW777549_PAB924C04_01977_Parthenium_ |
| | argentatum_cold_acclimated_bark_cDNA_library_(PAB)_Parthenium_argentatum_ |
| | cDNA_clone_PAB924C04,_mRNA_sequence |
| 473 | gi\|294681395\|gb\|GW783180.1\|GW783180_PBC054F05_07608_Parthenium_ |
| | argentatum_cold_acclimated_bark_cDNA_library_(PBC)_Parthenium_argentatum_ |
| | cDNA_clone_PBC054F05,_mRNA_sequence |
| 474 | gi\|294681374\|gb\|GW783159.1\|GW783159_PBC054D08_07587_Parthenium_ |
| | argentatum_cold_acclimated_bark_cDNA_library_(PBC)_Parthenium_argentatum_ |
| | cDNA_clone_PBC054D08,_mRNA_sequence |
| 475 | gi\|294681372\|gb\|GW783157.1\|GW783157_PBC054D06_07585_Parthenium_ |
| | argentatum_cold_acclimated_bark_cDNA_library_(PBC)_Parthenium_argentatum_ |
| | cDNA_clone_PBC054D06,_mRNA_sequence |
| 476 | gi\|294681365\|gb\|GW783150.1\|GW783150_PBC054C11_07578_Parthenium_ |
| | argentatum_cold_acclimated_bark_cDNA_library_(PBC)_Parthenium_argentatum_ |
| | cDNA_clone_PBC054C11,_mRNA_sequence |
| 477 | gi\|29468132\|gb\|GW777497.1\|GW777497_PAB923B03_01925_Parthenium_ |
| | argentatum_cold_acclimated_bark_cDNA_library_(PAB)_Parthenium_argentatum_ |
| | cDNA_clone_PAB923B03,_mRNA_sequence |
| 478 | gi\|294681286\|gb\|GW777462.1\|GW777462_PAB923B04_01890_Parthenium_ |
| | argentatum_cold_acclimated_bark_cDNA_library_(PAB)_Parthenium_argentatum_ |
| | cDNA_clone_PAB923B04,_mRNA_sequence |
| 479 | gi\|294681285\|gb\|GW777461.1\|GW777461_PAB923E01_01889_Parthenium_ |
| | argentatum_cold_acclimated_bark_cDNA_library_(PAB)_Parthenium_argentatum_ |
| | cDNA_clone_PAB923E01,_mRNA_sequence |
| 480 | gi\|294681284\|gb\|GW777460.1\|GW777460_PAB923B09_01888_Parthenium_ |
| | argentatum_cold_acclimated_bark_cDNA_library_(PAB)_Parthenium_argentatum_ |
| | cDNA_clone_PAB923B09,_mRNA_sequence |
| 481 | gi\|294681261\|gb\|GW777437.1\|GW777437_PAB923A04_01865_Parthenium_ |
| | argentatum_cold_acclimated_bark_cDNA_library_(PAB)_Parthenium_argentatum_ |
| | cDNA_clone_PAB923A04,_mRNA_sequence |
| 482 | gi\|294681250\|gb\|GW783114.1\|GW783114_PBC053H07_07542_Parthenium_ |
| | argentatum_cold_acclimated_bark_cDNA_library_(PBC)_Parthenium_argentatum_ |
| | cDNA_clone_PBC053H,_mRNA_sequence |
| 483 | gi\|294681220\|gb\|GW783084.1\|GW783084_PBC053E11_07512_Parthenium_ |
| | argentatum_cold_acclimated_bark_cDNA_library_(PBC)_Parthenium_argentatum_ |
| | cDNA_clone_PBC053E11,_mRNA_sequence |
| 484 | gi\|294681216\|gb\|GW783080.1\|GW783080_PBC053E07_07508_Parthenium_ |
| | argentatum_cold_acclimated_bark_cDNA_library_(PBC)_Parthenium_argentatum_ |
| | cDNA_clone_PBC053E07,_mRNA_sequence |
| 485 | gi\|294681215\|gb\|GW783079.1\|GW783079_PBC053E05_07507_Parthenium_ |
| | argentatum_cold_acclimated_bark_cDNA_library_(PBC)_Parthenium_argentatum_ |
| | cDNA_clone_PBC053E05,_mRNA_sequence |
| 486 | gi\|294681151\|gb\|GW777406.1\|GW777406_PAB922D05_01834_Parthenium_ |
| | argentatum_cold_acclimated_bark_cDNA_library_(PAB)_Parthenium_argentatum_ |
| | cDNA_clone_PAB922D05,_mRNA_sequence |
| 487 | gi\|294681117\|gb\|GW777372.1\|GW777372_PAB922D01_01800_Parthenium_ |
| | argentatum_cold_acclimated_bark_cDNA_library_(PAB)_Parthenium_argentatum_ |
| | cDNA_clone_PAB922D01,_mRNA_sequence |
| 488 | gi\|294681117\|gb\|GW777372.1\|GW777372_PAB922D011_01800_Parthenium_ |
| | argentatum_cold_acclimated_bark_cDNA_library_(PAB)_Parthenium_argentatum_ |
| | cDNA_clone_PAB922D01,_mRNA_sequence |
| 489 | gi\|294681109\|gb\|GW777364.1\|GW777364_PAB922C08_01792_Parthenium_ |
| | argentatum_cold_acclimated_bark_cDNA_library_(PAB)_Parthenium_argentatum_ |
| | cDNA_clone_PAB922C08,_mRNA_sequence |
| 490 | gi\|294680332\|gb\|GW786118.1\|GW786118_PBC087F05_10546_Parthenium_ |
| | argentatum_cold_acclimated_bark_cDNA_library_(PBC)_Parthenium_argentatum_ |
| | cDNA_clone_PBC087F05,_mRNA_sequence |
| 491 | gi\|294680324\|gb\|GW786110.1\|GW786110_PBC087E05_10538_Parthenium_ |
| | argentatum_cold_acclimated_bark_cDNA_library_(PBC)_Parthenium_argentatum_ |
| | cDNA_clone_PBC087E05,_mRNA_sequence |
| 492 | gi\|294680319\|gb\|GW786105.1\|GW786105_PBC087D11_10533_Parthenium_ |
| | argentatum_cold_acclimated_bark_cDNA_library_(PBC)_Parthenium_argentatum_ |
| | cDNA_clone_PBC087D11,_mRNA_sequence |
| 493 | gi\|294680301\|gb\|GW78607.1\|GW786087_PBC087C05_10515_Parthenium_ |
| | argentatum_cold_acclimated_bark_cDNA_library_(PBC)_Parthenium_argentatum_ |
| | cDNA_clone_PBC087C05,_mRNA_sequence |
| 494 | gi\|294680277\|gb\|GW786063.1\|GW786063_PBC087A05_10491_Parthenium_ |
| | argentatum_cold_acclimated_bark_cDNA_library_(PBC)_Parthenium_argentatum_ |
| | cDNA_clone_PBC087A05,_mRNA_sequence |
| 495 | gi\|294680260\|gb\|GW786046.1\|GW786046_PBC086G08_10474_Parthenium_ |
| | argentatum_cold_acclimated_bark_cDNA_library_(PBC)_Parthenium_argentatum_ |
| | cDNA_clone_PBC086G08,_mRNA_sequence |
| 496 | gi\|294680255\|gb\|GW786041.1\|GW786041_PBC086G05_10469_Parthenium_ |
| | argentatum_cold_acclimated_bark_cDNA_library_(PBC)_Parthenium_argentatum_ |
| | cDNA_clone_PBC086G05,_mRNA_sequence |
| 497 | gi\|294680202\|gb\|GW785988.1\|GW785988_PBC086B05_10416_Parthenium_ |
| | argentatum_cold_acclimated_bark_cDNA_library_(PCB)_Parthenium_argentatum_ |
| | cDNA_clone_PBC086B05,_mRNA_sequence |
| 498 | gi\|294680186\|gb\|GW785972.1\|GW785972_PBC085H10_10400_Parthenium_ |
| | argentatum_cold_acclimated_bark_cDNA_library_(PBC)_Parthenium_argentatum_ |
| | cDNA_clone_PBC085H10,_mRNA_sequence |
| 499 | gi\|294680182\|gb\|GW785968.1\|GW785968_PBC085H06_10396_Parthenium_ |
| | argentatum_cold_acclimated_bark_cDNA_library_(PBC)_Parthenium_argentatum_ |
| | cDNA_clone_PBC085H06,_mRNA_sequence |
| 500 | gi\|294680172\|gb\|GW785958.1\|GW785958_PBC085G07_10386_Parthenium_ |
| | argentatum_cold_acclimated_bark_cDNA_library_(PBC)_Parthenium_argentatum_ |
| | cDNA_clone_PBC085G07,_mRNA_sequence |
| 501 | gi\|294680162\|gb\|GW785948.1\|GW785948_PBC085F08_10376_Parthenium_ |
| | argentatum_cold_acclimated_bark_cDNA_library_(PBC)_Parthenium_argentatum_ |
| | cDNA_clone_PBC085F08,_mRNA_sequence |
| 502 | gi\|294680142\|gb\|GW785928.1\|GW785928_PBC085D11_10356_Parthenium_ |
| | argentatum_cold_acclimated_bark_cDNA_library_(PBC)_Parthenium_argentatum_ |
| | cDNA_clone_PBC085D11,_mRNA_sequence |
| 503 | gi\|294680119\|gb\|GW785905.1\|GW785905_PBC085B09_10333_Parthenium_ |
| | argentatum_cold_acclimated_bark_cDNA_library_(PBC)_Parthenium_argentatum_ |
| | cDNA_clone_PBC085B09,_mRNA_sequence |
| 504 | gi\|294680098\|gb\|GW785884.1\|GW785884_PBC084H12_10312_Parthenium_ |
| | argentatum_cold_acclimated_bark_cDNA_library_(PBC)_Parthenium_argentatum_ |
| | cDNA_clone_PBC084H12,_mRNA_sequence |
| 505 | gi\|294680070\|gb\|GW785856.1\|GW785856_PBC084F08_10284_Parthenium_ |
| | argentatum_cold_acclimated_bark_cDNA_library_(PBC)_Parthenium_argentatum_ |
| | cDNA_clone_PBC084F08,_mRNA_sequence |
| 506 | gi\|294680060\|gb\|GW785846.1\|GW785846_PBC0894E10_10274_Parthenium_ |
| | argentatum_cold_acclimated_bark_cDNA_library_(PBC)_Parthenium_argentatum_ |
| | cDNA_clone_PBC084E10,_mRNA_sequence |
| 507 | gi\|294680038\|gb\|GW785824.1\|GW785824_PBC084C10_10252_Parthenium_ |
| | argentatum_cold_acclimated_bark_cDNA_library_(PBC)_Parthenium_argentatum_ |
| | cDNA_clone_PBC084C10,_mRNA_sequence |
| 508 | gi\|294680031\|gb\|GW785817.1\|GW785817_PBC084C05_10245_Parthenium_ |
| | argentatum_cold_acclimated_bark_cDNA_library_(PBC)_Parthenium_argentatum_ |
| | cDNA_clone_PBC084C05,_mRNA_sequence |
| 509 | gi\|294680009\|gb\|GW785795.1\|GW785795_PBC084A05_10233_Parthenium_ |
| | argentatum_cold_acclimated_bark_cDNA_library_(PBC)_Parthenium_argentatum_ |
| | cDNA_clone_PBC084A05,_mRNA_sequence |
| 510 | gi\|294680009\|gb\|GW785795.1\|GW785795_PBC084A05_10223_Parthenium_ |
| | argentatum_cold_acclimated_bark_cDNA_library_(PBC)_Parthenium_argentatum_ |
| | cDNA_clone_PBC084A05,_mRNA_sequence |
| 511 | gi\|294679929\|gb\|GW785715.1\|GW785715_PBC083B05_10143_Parthenium_ |
| | argentatum_cold_acclimated_bark_cDNA_library_(PBC)_Parthenium_argentatum_ |
| | cDNA_clone_PBC083B05,_mRNA_sequence |
| 512 | gi\|294679926\|gb\|GW785712.1\|GW785712_PBC083A12_10140_Parthenium_ |
| | argentatum_cold_acclimated_bark_cDNA_library_(PBC)_Parthenium_argentatum_ |
| | cDNA_clone_PBC083A12,_mRNA_sequence |
| 513 | gi\|294679879\|gb\|GW785665.1\|GW785665_PBC082E07_10093_Parthenium_ |
| | argentatum_cold_acclimated_bark_cDNA_library_(PBC)_Parthenium_argentatum_ |
| | cDNA_clone_PBC082E07,_mRNA_sequence |
| 514 | gi\|29467853\|gb\|GW785639.1\|GW785639_PBC082C05_10067_Parthenium_ |
| | argentatum_cold_acclimated_bark_cDNA_library_(PBC)_Parthenium_argentatum_ |
| | cDNA_clone_PBC082C05,_mRNA_sequence |
| 515 | gi\|294679832\|gb\|GW785618.1\|GW785618_PBC082A07_10046_Parthenium_ |
| | argentatum_cold_acclimated_bark_cDNA_library_(PBC)_Parthenium_argentatum_ |
| | cDNA_clone_PBC082A07,_mRNA_sequence |

| Sequence | | TM | | TM | Product | |
|---|---|---|---|---|---|---|
| ID | Forward | Forward | Reverse | Reverse | Size | Repeat |
| 1 | GAAACCATGGCAAAACCC | 58.745 | TGCTGACTGATACTGCTGGA | 58.081 | 210 | (TAA)4- |
| | | | | | | (CCA)4 |
| 2 | ATCTGAACCCAGAACCCTTC | 58.021 | GGTTATCTGAGAAGCCAGCA | 58.048 | 285 | (ATG)4 |
| 3 | CTCAAAAGCACCACCCTTAG | 57.486 | GTGGTGAGTCGTTTTTGGTC | 58.029 | 209 | (CAC)5- |
| | | | | | | (CCT)5- |
| | | | | | | (CCT)5 |
| 4 | CTCTTCGAACGGTCTGAAAC | 57.514 | ATAGGGGATTCTCGTAACCG | 58.02 | 102 | (AAT)5 |
| 5 | TACCCTCCTCAGACCCTACC | 58.05 | GATTTCGGGCTAAAGAGGTC | 57.875 | 221 | (ATG)4 |
| 6 | CACCTACCTTCTTTTGGCCT | 58.318 | GCAACGTTAAAGACGGTGAC | 58.283 | 177 | (GA)7 |
| 7 | CCGTAGGTACAACACCGAAC | 58.008 | GGGGGAGAAAGAGAGAAACA | 58.296 | 333 | (ATC)4 |
| 8 | GAACTGGCTGTGAATGCTCT | 58.028 | ACTTTCACACAACGTCCTCC | 57.609 | 166 | (AC)8 |
| 9 | GAACTGGCTGTGAATGCTCT | 58.028 | ACTTTCACACAACGTCCTCC | 57.609 | 166 | (AT)7 |
| 10 | GCTCCAGTACGATTACGACG | 58.406 | GGCACTTTAAGGAGCTGTGA | 58.104 | 174 | (ATT)4 |
| 11 | CATCACCTTCTTCATCCCAC | 57.91 | CATTAGGAGGTGGAAACCCT | 57.975 | 480 | (TCA)4 |
| 12 | CCCTCCTCAGACCCTATCAT | 57.993 | TGACATAAGGAGCCCAGTGT | 58.158 | 279 | (ATG)4 |
| 13 | CCCAATGCTCTTCCTTCTTC | 58.861 | AGAGGAATTACTGAGGGCGT | 57.881 | 275 | (CCA)4 |
| 14 | GTCTTCCTTCCGCATGTCT | 57.793 | GATCAGCGGCAAGTAGTGTT | 57.979 | 148 | (TC)6 |
| 15 | TCATAGCCAGAGAGACTGCC | 58.129 | GCTTCTCTAAACTGGCCACA | 58.104 | 147 | (GCT)4 |
| 16 | GGAGGTCAGGAACTCAGGAT | 58.101 | GTGCTTGCTGTCAGCTTATG | 57.244 | 352 | (CTT)4 |
| 17 | CCATCATATGTCCCAAAGAG | 58.071 | TGAGCTAACGCCACATCT | 55.278 | 293 | (CTT)4 |
| 18 | TCCTCCTAAATCCTCGTTCC | 58.187 | CGGCTGTGAAGTGAAATACC | 58.228 | 460 | (TAT)4 |
| 19 | ATCTTCTTCTTCGTCCCACC | 58.173 | ACCTCGGGGATTTCTACAAC | 57.974 | 145 | (CTT)4 |
| 20 | CCACTCCTCCAAAACCTTCT | 58.228 | GAACCATCGGTACGATTCTG | 58.019 | 354 | (TAC)4 |
| 21 | GGGTTTCATCCTCCTCACTT | 58.021 | TGGGATAACTCTAGCCACCA | 58.173 | 463 | (AGG)5 |
| 22 | CATTATCAGTAGGCTGGGGA | 57.641 | AGCCGATGTACACTTCG | 57.98 | 480 | (TTA)4 |
| 23 | GCCGAAGAAGACCTGTATGT | 57.824 | ATCTCCTCCTTCAACGCTTC | 58.173 | 187 | (TAA)4 |
| 24 | GTTCTTCTCCGGTTATGGGT | 57.974 | CAGTACCAGTTCCAGTTCCG | 58.215 | 240 | (ATA)4 |
| 25 | AGGACAGCTACACAAGTGGC | 57.958 | CACTCCCACCATTGAAAGTC | 57.971 | 397 | (AAG)5 |
| 26 | GACGCCGTATTACAATCCAC | 579 | CAACCCCATCAACAACCAC | 59.641 | 260 | (TCG)4 |
| 27 | CACCCGAGTCCATCAAAA | 57.927 | ATAGAGCGTGGCATAGTTGG | 57.877 | 157 | (GAT)4 |
| 28 | CCCACTGGAACCCTAATCTT | 57.975 | TACAGCTCCGACTGACAACA | 57.977 | 183 | (GTG)4 |
| 29 | ATGTATGATGCTAGCGGCTC | 57.969 | CAGGATCTGGAAACTCATCG | 58.23 | 310 | (AAT)4 |
| 30 | GTATGAGGGCTTCAGGGATT | 58.067 | CACCAATAGAAGTGGCAACC | 58.076 | 349 | (GAT)6 |
| 31 | CCCTTGCTTCCACATAACAG | 58.228 | ACCAACCTCATCTTCTGACG | 57.71 | 426 | (TGC)4 |
| 32 | ACTGCCAGAAGTGTTTCCAG | 57.93 | ACCTGATTCCGACCTAGACC | 58.049 | 266 | (AAC)4 |
| 33 | AAGTACCGACCTGGTTGACA | 58.059 | ACATGCTCCACCTCCTTGTA | 58.158 | 378 | (TGA)6 |
| 34 | AAGAGGAACTCAAGAAGCG | 58.67 | AGACTGTGATTCAGGCTTCG | 58.029 | 497 | (GAT)5 |
| 35 | AGTGGATCATGTCTTGCTGC | 58.832 | ACCTTCACTCACAAAGCTCG | 58.087 | 188 | (CCA)5 |
| 36 | CTTGCCCTTTCTGTACGCTA | 58.201 | GCCATCTTTGGAGTAAAGGG | 58.646 | 478 | (TGAG) |
| | | | | | | GA)4 |
| 37 | CACTCCTTCAGTGACCTCCT | 56.817 | CAAACAGGAGAGCAGAAAGC | 57.826 | 224 | (TTGA)4 |
| 38 | GGCAAAATCAGGTACTTCCC | 58.504 | GCTCTCAAGTTACTGCAGCC | 57.853 | 194 | (TTA)4 |
| 39 | GAACCATTATGCAGCTGGTC | 58.173 | GCCTCCAGTAGTGGTAGCAA | 57.98 | 192 | (GGT)7 |
| 40 | CGATACACCTTCAAACGGAG | 58.228 | GACGGTGGAGATGGTTGTAG | 58 | 203 | (CCG)4 |
| 41 | ATACAGGTCACTGGCTCTCG | 57.9 | ACCACCTTATGGCAAGGACT | 58.525 | 410 | (GAA)5 |
| 42 | TCCTTACAAACCCAACCC | 55.746 | TCTTCGAAACCGTAGACCAG | 57.952 | 474 | (AC)6 |
| 43 | CGAGGTACTTCCACACCAAC | 58.059 | AAGTTCGGCCCTATACTGCT | 57.985 | 217 | (TGC)4 |
| 44 | TAGGGTTTTCTCTGCGACTG | 58.105 | TTATGCTGACTTCCTCAGCC | 58.048 | 262 | (CAG)4 |
| 45 | TGTGCTCGGCAGTAGGTAAT | 58.406 | ATCGTTGTCCACTAGCTTCG | 57.98 | 450 | (TGC)5 |
| 46 | TGGAGGAGGGTTAAGGCTA | 57.759 | CGGTTAGGTCATTGATGGAG | 58.02 | 170 | (TCT)5 |
| 47 | CAACCACTCCTCCACATCTC | 58.052 | ACGGTGCACTGCTTACTTTC | 58.036 | 484 | (ATA)4 |
| 48 | CCCCCTCTTTGGTTGTAT | 57.975 | TTGTGTATCGACAGCTGCAC | 58.439 | 491 | (ATT)4 |
| 49 | CGAGCTCATCTTCGATCTTC | 57.706 | CCCATGAGATGACTTCAACC | 57.91 | 241 | (ATC)4 |
| 50 | ACGTCGCTTAAAGACACAGC | 58.187 | AGTGTCAAACGTGCCTAACC | 57.729 | 189 | (ACA)4 |
| 51 | GTCATGGTATCAGAGCCTTG | 55.672 | AGGGAGTAGCAAGGGAAGAA | 58.002 | 472 | (ACC)4 |
| 52 | CCTACCACCACCACCATAAA | 58.201 | GGGAGTAGCAAGGGAAGAAG | 58.002 | 398 | (GCC)5- |
| | | | | | | (GCC)4 |
| 53 | CCGACCTAAGCAAATCACAC | 58.228 | CATCTCCCTGTAATCGCTTG | 58.325 | 307 | (AAT)4 |
| 54 | CGTTCTAGTACCCCTCCCTC | 57.734 | TCAAATAGGTCGGAGACAGAC | 57.893 | 318 | (AAT)5 |
| 55 | TCAGAGCCTTGAAGACAACC | 58 | CACATGTATGTTGGTGACGCT | 58.246 | 146 | (ACC)4 |
| 56 | CACGGAGTGGTGATCAAAAGG | 58.13 | CTTTGTCCTTGCTCGTCTGT | 58.087 | 374 | (TGA)4 |
| 57 | ACTTGCCGTATGTGTGTGTG | 58.038 | CGGTAACCTGGAGGGTATTT | 57.93 | 310 | (AT)6 |
| 58 | CACACCCACTAGATCCATCC | 57.779 | CTGCTACAAGAACCGCTGAT | 58.132 | 399 | (TTA)4 |
| 59 | TCACTCTGGGTTCTTTCACC | 57.679 | CTTGTCGAATATCTGCCCAC | 58.173 | 228 | (TGG)5 |
| 60 | TCCCTCTCTTTACCACCCTT | 57.706 | CCGGTATTCTAAACTCCGGT | 58.072 | 398 | (CA)8 |
| 61 | CCTTTCACTCCACACACACA | 580.63 | GATCTCGACCTAGACATCCTTG | 57.881 | 456 | (TAA)4 |
| 62 | GGTGACAACAGGAAGTGAGC | 58.275 | ACCTCATCTTGTGGGTGGTA | 57.84 | 222 | (AAC)4 |
| 63 | CAGCCAGCAGTTAACCAGAT | 57.98 | CACCCAAAGAATAGCAGGTG | 58.228 | 210 | (GCT)4 |
| 64 | ACCTGTCAGAACAAGCCAA | 57.93 | TATTGAGGCCTCCTTCTCCT | 57.946 | 172 | (GATA)4 |
| 65 | CACAGACAGTCCCAAATCCT | 57.548 | ACAAGGTTCCTCATCTTCCC | 58.021 | 313 | (ATT)4 |
| 66 | CTTCCGATCTAGACGACGAA | 58.048 | CCACGTGTCAAGCCTTATTC | 58.228 | 128 | (GAA)4 |
| 67 | GTTGCTGTCGAAAACCCTG | 59.278 | TCAGCATGTTCCCTCTCTTC | 57.94 | 473 | (TTA)5 |
| 68 | ACTCTACCTCCATGCTGACG | 57.9 | CCTGGTTCAGAAGATTGCAC | 58.287 | 429 | (AAG)4 |
| 69 | GCCCTATGCTTCACTCCATA | 57.791 | AGGCTGAAGGTCTTCTCGTT | 58.133 | 414 | (GCAA |
| | | | | | | AC)4 |
| 70 | GAAGAGAAAGCAGCAGCGTA | 58.555 | TACCGACCCCTATCCCTATC | 57.821 | 440 | (CTT)6 |
| 71 | GAGGCTCAGGTCTTCCTTCT | 57.953 | CTCCTCCTTCTTCCTCT | 56.689 | 376 | (GAT)5- |
| | | | | | | (TAGA |
| | | | | | | GA)5 |
| 72 | ACTGGTCTTCGTTGCTGAAG | 58.087 | AAAGAAAGGGAGACAGGCAC | 58.377 | 416 | (TG)8 |
| 73 | CCCTCAAACCAAATCACTCC | 59.381 | GTACGAGGTTTCGGAGTACG | 57.36 | 226 | (CAC)4 |
| 74 | GGAGCAGGAGCATATGAGAA | 57.99 | ATAATACTTCACCGCCTCCC | 58.019 | 188 | (TAT)5 |
| 75 | CAAAGGCAGCAGAGAAG AAG | 57.98 | CAGCAGTCTGGATCTCCCTA | 57.971 | 336 | (AAG)4 |
| 76 | GCAGCTTAGACCCAGTCAAC | 57.542 | TATCTTCCCTGCAGTACCCA | 58.173 | 463 | (CAC)4 |
| 77 | CCTCTCCGAGTCAAAACGTA | 57.952 | TGGAAGGTGCTGTAGGTGAT | 58.158 | 134 | (TAA)5 |
| 78 | ATACAGAGGGGTCCGAAAA | 57.974 | TACACGTGGCAGAACACACT | 57.137 | 464 | (TCG)4 |
| 79 | CCCTTCATCCTCTCTCTCATC | 57.853 | CAGCTCGGATGACTCAAACT | 58.029 | 390 | (AG)8 |
| 80 | CGTGTCAAAACTACGGTCT | 58.036 | AAGCTTTGACCGACTTCCTC | 58.525 | 135 | (CACG)4 |
| 81 | CAGGATCAGAATCGGATGAC | 58.011 | ATAGCAAGGCACCAGAACAG | 57.98 | 125 | (TGA)5 |
| 82 | GATTAGAGCGTCATCGGCTA | 58.092 | TGTCCTGCCTTCTCAAAGAG | 58.158 | 433 | (GAT)4 |
| 83 | GCATATCCGATCCAACTCAC | 57.96 | CTGTAACACCGAGTCCCATC | 58 | 227 | (GAT)4 |
| 84 | GTACACAATGGGAGCGTGTT | 58.498 | GCTAGCCTCACATAGACCGA | 58.076 | 391 | (GGT)4 |
| 85 | ATCTCTTCCACCACTCCCTC | 58.101 | CTGCCTAGCAGCTAAGTTGG | 57.961 | 378 | (ACA)4 |
| 86 | GGAAGTTAAAGATCCGGCG | 59.654 | ATCTTGGACAGGGGAGAATC | 57.964 | 259 | (ATG)4 |
| 87 | TCTGACTCCATCCAGCCTTA | 58.387 | AATGACTCTACCAGCGCAAC | 57.979 | 228 | (CA)9- |
| | | | | | | (GAA)4 |
| 88 | GAACCCGCAGCAATAGAA | 57.346 | ACCTCGGGGATTTCTACAAC | 57.974 | 326 | (TC)6 |
| 89 | AGAGACGTCAACGGAAACAC | 57.77 | GTTCCACGTCATGTCTAGGG | 58 | 244 | (ACC)5 |
| 90 | GATTACTCGTTCAACGGTCG | 58.228 | AGGAGGAAAAAGGGGGTACT | 58.074 | 327 | (TAG)6 |
| 91 | CAGTGAAAAGTGATGTCCCC | 57.971 | CAGAACGTGTCATCGTCAGA | 58.341 | 313 | (AAT)4 |
| 92 | CGTTGACGGAAGGATATGAC | 58.019 | GCAGCTAAAATCCTCCTCCT | 57.639 | 206 | (GCA)4 |
| 93 | GTTCTAGCGTGTGATGCCTT | 57.979 | GAAAGAACTTGTCCCAGAGC | 56.536 | 407 | (CTC)4 |
| 94 | CCATTGGCTCTCCTTCCTAT | 58.214 | TAGTCCTGAAAGCCACCTTG | 57.952 | 342 | (TTG)4 |
| 95 | CGATGGTCTCCACTACCAAC | 58 | AGAAACAGCCATGAGACCAG | 57.87 | 411 | (TTC)4 |
| 96 | CTGGGTACGCTCGCATAG | 57.968 | TGGAAGACCGAGTGGTGTAT | 58 | 343 | (GAG)5 |
| 97 | AACAGGCAGTCAGACACCAT | 58.142 | GCATCATAGGCTGCAGAGAT | 58.018 | 302 | (ATT)4 |
| 98 | GTCTCCAATGACCAAGATGC | 58.07 | CTAAAGCCTGCGAGTTATCG | 57.837 | 118 | (CCT)4 |
| 99 | GAGGAGGCTGCAGTTGATAA | 58.048 | GACCACCACGCAATGTTT | 57.85 | 173 | (TTGGTG)4 |
| 100 | TTCAGTGTGGTTGTGCGA | 58.202 | TTAGCTCAAGAGGTGATGGC | 58.048 | 159 | (GT)9 |
| 101 | ACCGGATTATCTCCGATCTC | 58.011 | CATCATGGTTGATAGGAGCC | 57.961 | 472 | (TGT)4 |
| 102 | CCCACACCAGTTCCTTACAC | 57.901 | GTGCTTGTGCACTATGGTTG | 57.764 | 481 | (CCA)4 |
| 103 | GAGGACATGGTCGGTGATAG | 57.94 | GCTACAAATGAGATCCCCG | 58.103 | 230 | (ATT)4 |
| 104 | CCCTAGGTTTTCCGATCAGT | 58.121 | CCGTAACACCACTGGATCTC | 58 | 281 | (CAG)5 |
| 105 | CCGAACAACATCCTGAACAC | 58.984 | CTTGCAACTGCATCTGTCAC | 57.976 | 499 | (AAT)4 |
| 106 | AACCGTCGTAACGATGACTC | 57.669 | TCTTCCTCTCCGTCACATTC | 57.779 | 164 | (CAC)5- |
| | | | | | | (GAT)4 |
| 107 | ACGACATCTGGAGATTCACG | 58.671 | AGCAAGTCTAGGGTTTCCGT | 57.936 | 282 | (GCT)6 |
| 108 | GGTTAGGCACGTTTGACACT | 57.729 | CGGTGTTGTAGTGAACGTGA | 58.172 | 423 | (ATA)4 |
| 109 | CCATGACAGAAGTCCTCCAC | 58.052 | CAGGTTCAAACTCAACAGGC | 58.342 | 352 | (ATA)4 |
| 110 | AACCCTCCTGGATTTGGAC | 58.777 | CGTCTCGTAAGCGTTTCATC | 58.529 | 259 | (AACA)4 |
| 111 | TCGTTCGTCTCTTCTGCTTC | 58.315 | ACACCTTCTCCTTCCCAAAC | 58.078 | 455 | (CTG)4 |
| 112 | TCTCTGTTGGATACAGGGGA | 58.071 | AAAGCAAGGACGAGTAGGGT | 57.936 | 319 | (TAA)4 |
| 113 | GTCGTTGCTGATACTGCCTT | 57.979 | GAACAGTAGACGCCGAAGAA | 58.104 | 134 | (TGC)4 |
| 114 | CTGTCTGGGAACAAAACAGC | 58.342 | TCACTCCCCCTTTCCTATTC | 53.04 | 417 | (CAT)4 |
| 115 | GGTTTGAGTTTTCCCCCA | 58.302 | CTTGGTACGTTCGGTATTGG | 53.027 | 173 | (TA)7 |
| 116 | CGTTGCTGTCGCTCTCTAA | 57.882 | GTGCAACTCATCTCTTGCCT | 53.028 | 391 | (TC)8 |
| 117 | GAGTTCGAACCAAACACCAC | 58.029 | TACCAACAACAACACCCTCC | 53.315 | 337 | (ATG)4 |
| 118 | GCTGAAGCTGACCACACTTT | 58.087 | GCCCTATCAGAAACGAACAC | 57.265 | 434 | (TGG)4 |
| 119 | GAGCCTGAGAATGTTGCAGT | 58.028 | GTTTCTTAGGCGGCTTCTCT | 57.839 | 103 | (GAA)5 |
| 120 | CAGAGCTATCCTCAATCGGA | 57.991 | CGTTTTGTACCGTTACGGAG | 53.227 | 218 | (CAATTT)4 |
| 121 | CCTTTCAGGTGTCTGCACTT | 57.93 | CGTATTTCTCCACACCAACC | 57.923 | 253 | (TGA)6 |
| 122 | CCTGCACTCAGCTCCACTAT | 58.058 | GAACTAGCGGCTTTAGCCTT | 57.939 | 257 | (TGT)4 |
| 123 | CTTACTACGCCTCAGCATGG | 58.554 | TAGAATCTCATCTCCTGCGG | 57.991 | 491 | (TCC)4 |
| 124 | CAAACAGACCCACCAGTTGT | 58.463 | GTGTAGCCACATCAACCTCC | 57.999 | 414 | (TAT)4 |
| 125 | CGGTCAATCTTTGCGTTC | 57.743 | GGTGATGACCACTCCACTTC | 57.886 | 165 | (CAA)6 |
| 126 | CCTCTACACTTGCTAACCGC | 57.663 | GGTTGGTGTCTTGAAGGAGA | 57.679 | 128 | (TCC)4 |
| 127 | ATCCTCCTCCTCATCTGACC | 58.043 | CTCAACAGTCCACAGCCTTT | 57.93 | 338 | (GATTCC)4 |
| 128 | TATTCGAGAGTCGTTGAGCC | 58.048 | AAGAAACCCCCACTCTCATC | 58.021 | 182 | (GGATCA)4 |
| 129 | CTTCTTCCTCTTCACACACG | 55.987 | GCTTGTGTTGTGGTGGTGTA | 58.007 | 126 | (TTACAG)5 |
| 130 | CACGCCTTCTACGAGACATT | 57.98 | GATGAGGGAGATCTGTTTGG | 57.063 | 453 | (TCT)4 |
| 131 | CCCAACTAACCACCTTCTCTC | 57.776 | GGGTGGGTTTGAAGGAGTT | 58.828 | 111 | (CT)6 |
| 132 | CGACTTAACCCCTGGTTAGC | 58.718 | TTGGGTCTGGTACTGTTGGT | 57.901 | 150 | (CAA)4 |
| 133 | CCACCCAACCAATGTCTTAC | 57.768 | CAGCCTAAAAGATCCCTTCC | 57.876 | 426 | (AAC)6 |
| 134 | CACCTATTCTCGCACACACA | 58.276 | GGCCAACAAGTTATGTGAGG | 58.076 | 491 | (TTA)4 |
| 135 | CTGGTGGTATAACGGAGGTG | 57.952 | CAACACAGCACCACCAAA | 57.463 | 117 | (GTG)4 |
| 136 | CATCCGTAACACCACCAAGA | 59.415 | TGGTCTAACGGTGGTTGAGT | 58.059 | 184 | (CAC)4 |
| 137 | ATTCCGAGAGAGTTTGGACC | 58.173 | TGCTGTGACGAGACCAACTA | 57.977 | 233 | (CAC)4 |
| 138 | TACCCACAGTGGCTTCAACT | 58.215 | CCTCTAGTGCCCATCAAAGA | 57.894 | 181 | (AAT)5 |
| 139 | CGATCCCCCTTTCTCATATC | 58.401 | CTTTGGAGTACCGAAACCTG | 57.334 | 426 | (TTC)4 |
| 140 | CCTACGCTTTCTCTCATCCA | 58.048 | TTCTTGTGCGGCTACCTATC | 57.999 | 235 | (TCT)4 |
| 141 | GAGGTTCTGATCGTGAATGG | 58.071 | CGTGTCTCCCAATTTACCTG | 58.077 | 241 | (TCT)5 |
| 142 | TCACCTTCATACCCACCAGT | 57.84 | CCATGTAGAAAGATCCAGCG | 58.325 | 238 | (CAT)4 |
| 143 | ATAAACCCTCAACCCACTCC | 57.826 | CAACCTCAACAACCATCCTC | 57.971 | 439 | (CAAA)4 |
| 144 | TCCCAGGAGGTTCCTAAATC | 58.04 | ACCAGGATGTCAGATGCAGT | 58.082 | 263 | (TTG)4 |
| 145 | GCGTTAATCCGTTCCCTATG | 59.433 | AACACTAAGCCTCGGTTCCT | 57.936 | 373 | (CAA)4 |
| 146 | GCACTGCGTCACCTTACTTT | 58.036 | AACACCAATCACCTAGAGCG | 57.826 | 305 | (CT)9 |
| 147 | AGAAGAACTCGAAGAAGCCC | 57.732 | GATGAGGGAAGTGTGGTTGA | 58.482 | 159 | (CTC)4 |
| 148 | ACTGTGCTGAGAATGGGTGT | 58.142 | GCATAACAGGAAGAGCTCCA | 58.048 | 408 | (GGT)4 |
| 149 | CATCTCCGATCGATGGTAAC | 57.961 | AGGAATATGAAGAGGGTGGG | 57.919 | 363 | (TCA)5 |
| 150 | CCCCAGACCCTACCAATAAC | 58.24 | AGTCCCACATCAGTGCAAGT | 53.142 | 296 | (GTT)4 |
| 151 | CCCCAGACCCTACCAATAAC | 58.24 | AGTCCCACATCAGTGCAAGT | 53.142 | 296 | (TTA)4 |
| 152 | CAAGTACCGTTCTTCAAGCG | 58.579 | ATCAAAGTTACCCTCTCGGG | 58.121 | 391 | (ACA)4 |
| 153 | CGCCTCTCCAAACCTCTATT | 58.411 | ATAGGTACCGGGTTTTCAGC | 58.072 | 243 | (CAA)4 |
| 154 | GTTCGTTCGTCCATCGTATC | 58.019 | GGGTGAAGTATCCCTGCTTT | 53.121 | 401 | (ATT)4 |
| 155 | GCTACACTTTTTGCACGGG | 59.332 | ACCCGTGATTGTCTGTTACG | 53.497 | 406 | (ATT)4 |
| 156 | AGGGTTCTACACAAACACCG | 57.576 | TCGGTACTATCCACCTCCAA | 58.02 | 333 | (AGTTTT)4 |
| 157 | AGGCTATGAAGCTCCTGGAT | 57.973 | GAGGAGCAGACTTATCTAGCCA | 57.866 | 320 | (TGG)4- |
| | | | | | | (TGA)4 |
| 158 | ACGAAGACGACTCAGCAAAC | 58.087 | GAAGTATGTCTCTGCCGTGG | 58.315 | 122 | (CTT)4 |
| 159 | CCCCACTTTGCTACATTACG | 58.174 | TAATCTGCTCGTGCCCTAAC | 57.999 | 192 | (CAT)4 |
| 160 | TGCTGTCCCTTAGTTCTTGC | 58.104 | CAGGCTTGTGGTGATGGT | 57.934 | 174 | (CAC)5 |
| 161 | AACCCTAACTCGATCAACCC | 57.974 | GCGTTAGGTTGTGAAAGCTC | 57.632 | 260 | (CAA)4- |
| | | | | | | (CGA)5 |
| 162 | GGGAAACGAGTCCCCTAATA | 57.993 | GGGCATTCTTTTCAGGAGTC | 58.717 | 164 | (TCA)4 |
| 163 | ACCGTTCACCTCTCTCCTTC | 58.314 | CATCTGATATTCTGGGGCAG | 58.117 | 413 | (AAG)6 |
| 164 | GGGGACATACCATCACAGAA | 58.201 | CCAACAACACCACCCAAC | 58.108 | 230 | (AC)6 |
| 165 | CATCTTGAGGCTTGGTCTGT | 57.87 | AAAGGTTGTCTTGTGGGACC | 58.891 | 317 | (TG)6 |
| 166 | GTTCAAGCAGCTCCAACAGT | 58.087 | GAAGGAGATGGGTTGGTCTT | 58.021 | 207 | (GAG)4 |
| 167 | GTTCGGGTACGGTAAAACCT | 57.983 | ATAGCATGAACCACGAGTCC | 57.607 | 231 | (TTC)4 |
| 168 | CACACCCCTCTGATGAAATC | 57.91 | GTGTGCTGCTAAACGAAGGT | 58.036 | 479 | (TCT)5 |
| 169 | CAGAAACCACCCAACTCAAG | 58.187 | GAGAACATGTTGGGTGAAGG | 57.971 | 300 | (CAC)4 |
| 170 | GTGAAGTTGCAGCAGTTCCT | 58.087 | GCCCAGCAGCATATACCTTA | 57.898 | 339 | (GCT)4 |
| 171 | CTCATCCCTTCAAAACCC | 55.352 | GGTTTGGAAACTGGAGGTCT | 58.078 | 136 | (AAG)4 |
| 172 | TTAAGGTGGCTATGGAGCAG | 57.999 | AGGTCCGTGTTTTCCCTAAC | 58.029 | 199 | (GTG)5 |
| 173 | CGCGTACCGTATTACAACCT | 57.746 | GCAGAAGACGGAAGACTTTG | 57.671 | 434 | (ATA)5 |
| 174 | TGCTCCGTCTAAGAAAGCTC | 57.452 | CCTATAACCACCACGTCTCTC | 58 | 294 | (GCC)4 |
| 175 | GTACGGAGCTTTTGGAGTCA | 57.951 | CCGGCAAACTGCTAAGTCTA | 58.201 | 193 | (ATG)4 |
| 176 | CTACTTTTCCCCGGGTT | 58.895 | ACCCGTGATTGTCTGTTACG | 58.497 | 413 | (ATT)5 |
| 177 | GCGATCCAACAAACCATC | 57.338 | GTGGAGAGCTCGAGGTACAA | 58.029 | 154 | (TC)7 |
| 178 | CGATGGGAGTTCTCTAACGA | 57.894 | CAGCTGAACGTTTTCTCTCC | 57.671 | 337 | (CAG)5- |
| | | | | | | (GGT)4 |
| 179 | GATAAGATCCGCCGTCAAC | 58.102 | GAAGCAGAGAAACCTCCTCC | 58.049 | 500 | (AAC)4 |
| 180 | TCACCCCAAAACGATGTC | 57.755 | CACTAGAAACCAATCCACCG | 58.077 | 435 | (TCT)4 |
| 181 | CCTAACCCCACCTCTTGTTT | 58.029 | ATGAGGAGAGAGAGGCTGGT | 58.002 | 373 | (AG)6 |
| 182 | TCTACCGACAACCAAACCC | 58.401 | CCCTAACTTTGCTCCCTCTC | 58.002 | 247 | (AAG)5- |
| | | | | | | (CAT)4 |
| 183 | GCACACACACAGACACCAAT | 57.921 | CTGGCCAGATCTGTGTATCC | 58.1 | 244 | (CAA)4 |
| 184 | CCTCCCCTGTCTTTTCCTTA | 58.24 | AAAGATGGGATTCCAGGC | 57.422 | 314 | (CT)7 |
| 185 | TTAAGGAGGCGATAGTGACG | 57.999 | AGCTTTAGGAGGAGCTTTGG | 57.841 | 350 | (AAG)4- |
| | | | | | | (CAG)4 |
| 186 | ACTGCCACCAATAACACCAC | 58.343 | GGAATGGGAGTTCGGTTAGT | 57.974 | 114 | (ACC)4 |
| 187 | GTAGGGGTGTTCATTTGTGG | 57.768 | TACCCTCCTCAGACCCTACC | 58.05 | 211 | (CAT)5 |
| 188 | AGGTCCAACCCTTGGATAAG | 57.975 | GTGGTTTGGAGGTGAAGGAT | 58.842 | 310 | (CTTC)4 |
| 189 | CGTTGCAGAAGCTAGAAAGG | 57.934 | TCGCGTGATCTCTTCTGACT | 58.699 | 232 | (AAG)4 |
| 190 | GTGTCGTTTTGTAGATCGCC | 58.229 | GAATACAACTCTCCGACCGA | 57.737 | 190 | (CGA)4 |
| 191 | CGAGTGTCCAGGATAACGAT | 57.61 | ACAGTCTCCCACGTGTCATT | 57.979 | 354 | (ATA)6- |
| | | | | | | (AGG)4 |
| 192 | GTTTCCAACACTCAACCCAC | 57.87 | GCCTTCTTCCGTAGCTCTCT | 57.908 | 196 | (GT)8 |
| 193 | GTTTCCAACACTCAACCCAC | 57.87 | GACTTAAGCCCCAATTCCTC | 57.73 | 220 | (GA)8 |
| 194 | CTGGTAAAAAGGGTTCTCGG | 58.692 | GAATGATCGATTGCAGGC | 57.61 | 293 | (GAA)4- |
| | | | | | | (TGG)6 |
| 195 | CAAAGACCCACAAGCCAA | 58.156 | CACCTCCGTAGTCTTCTCCA | 57.871 | 322 | (AAT)5 |
| 196 | GTGTTCACAGGTCCCGTTAG | 58.059 | CTATTCACTCATTCCACCGC | 58.173 | 480 | (TAA)5 |
| 197 | CCATCTCCAGCATCCTAATG | 58.117 | GTGTGGTAGTGTGGGTGGAT | 58.112 | 201 | (CCA)4 |
| 198 | GTGTAGGGTTGCAGAGGTGT | 57.639 | AACGAGCTGCAATACCTGAC | 58.283 | 293 | (GAA)4 |
| 199 | GAATATGGGAGCAGTTGGTG | 58.019 | CTCAGTGTGGTTCCAAGACC | 58.113 | 403 | (CAG)4 |
| 200 | AGGAGGTCATGAAAGAAGGG | 58.173 | GTCGGGATTCATCTCTGCTA | 57.834 | 271 | (TGC)4 |
| 201 | CCACACTTAACTCCACGTGTC | 58.13 | GTCACTCCGCTTTGAAGTTG | 58.497 | 462 | (CTT)4 |
| 202 | ATCCTTCGGATAGTCACCC | 57.918 | GGATATGTTCAGCAAGTGGC | 58.173 | 403 | (GAT)4 |
| 203 | GTGTGAACCTTCACGGACTC | 58.112 | GTGACGACGGACGAATTAAC | 58.076 | 354 | (TAA)4 |
| 204 | CAAAGGGAGAAGGGGATAT | 58.451 | AGCCCTCTGAAACTGATTCC | 58.324 | 123 | (TCA)5 |
| 205 | TGGAACAGATGTCGGGTAGT | 58 | ACTGTTGCTTCTGCTTGGAC | 58.087 | 446 | (GTG)4 |
| 206 | GATCAAATATGGAGACCGCC | 59.348 | TAATCCGTATGCACTAGCCC | 57.752 | 289 | (CCA)5 |
| 207 | CAGTCGCCAGTTATTCAACC | 58.228 | AAAACCTCCAAGACCTCACC | 58.078 | 223 | (TCT)5 |
| 208 | CTTCATCGTCCGTACCAACA | 59.566 | GGAATTAGCTCTGCCTGATG | 57.515 | 435 | (TAC)6 |
| 209 | CGGCGAGAGAGAGACAGTAG | 57.93 | AATATCCTCCGTAACGCTC | 58.444 | 295 | (CT)9 |
| 210 | GTCTCCGTCAACGTCATCTC | 58.216 | CTTGTAACGGGATCACAACC | 57.923 | 442 | (TAA)5- |
| | | | | | | (TTA)4 |
| 211 | GGGACTGATATTGTGGTTGC | 57.863 | TCTCTCTCCCCCTGAGATTT | 57.839 | 488 | (TGC)4 |
| 212 | ACCGTCCTATCTCCATCTCC | 57.992 | ACTGACTCAACCGCACTCTC | 58.009 | 395 | (AAG)5 |
| 213 | TGTGCTGCTCATAGAACCTG | 57.571 | GGAAGGGAGGAGAGTGAGAG | 57.973 | 232 | (CT)7 |
| 214 | GGGCTCGAATCATAGAGTCA | 57.834 | CGAGCTCTTCATCTTCCTCA | 58.258 | 362 | (GAA)4 |
| 215 | CTCCGTCATAACACTCCGTC | 58.158 | CTCATATTCCCTCACAAGCG | 58.325 | 409 | (AAC)4 |
| 216 | GTCTTCTACGTTCTCCGGC | 57.465 | AAGACCAGCAAGACCACAAG | 57.93 | 319 | (TGC)4 |
| 217 | GGGGATTTGCAGTTGGAT | 58.274 | CGCCTTATCATGAGACCTTG | 58.325 | 411 | (AGA)4 |
| 218 | CTCTGCAACATCTAGGCAGC | 58.777 | CAGGAGCTACTCCACCAGAA | 58.03 | 198 | (TC)6 |
| 219 | CATCACCTTCTTCATCCCAC | 57.91 | CAAACCCTCCACTTAACCCT | 58.029 | 291 | (TCA)5 |
| 220 | GCTACCGTTTCGTACAACCA | 58.701 | GCTCATCAAACAGCTAGGT | 58.028 | 480 | (TCT)4 |
| 221 | GGTATGGAGCGTACTCCGTT | 59.08 | GGTGCAGCTGAACTCGTATT | 57.979 | 244 | (CCA)4 |
| 222 | AGTCGATCATGTCTTGCTGC | 58.99 | ACCTTCACTCACAAAGCTCG | 58.0871 | 188 | (CCA)5 |
| 223 | GTCACGGTTCAGGAAACATC | 57.97 | CTAAACCGCCACATCTCACT | 57.826 | 185 | (GAT)5 |
| 224 | CCTTCATTCAATCTAGCCTC | 57.668 | GTGATGGTTCCTATGATGCC | 57.803 | 424 | (ATC)4 |
| 225 | CTCAACAGGCTGCTTATGGT | 57.98 | CCAGGTACAAATCACGCAAG | 59.195 | 497 | (CAC)4- |
| | | | | | | (CTG)4 |
| 226 | TTGCCCTCTGGTGTTTAGTC | 57.797 | CTGAGAGAAGCAAGCACTCC | 57.9 | 326 | (AG)7 |
| 227 | TCAGGGTAAACCTTACTGCG | 57.907 | GCCTTGAGTCTTTGCTTCTG | 57.826 | 179 | (TGA)4 |
| 228 | GGATGTTGGCTGACTTTACG | 58.228 | TGGTGGTCTTACTTCTGCTG | 56.46 | 491 | (AGA)4 |
| 229 | GAACGGTGGATACAATCAGC | 58.019 | GACATATCGTCCTCATTCGG | 57.961 | 473 | (GGT)4 |
| 230 | CGAAGGTGATCGAGAAGAT | 58.387 | ATAGGTCGATCGTCCCTTTC | 58.067 | 264 | (GGT)4 |
| 231 | GGGAGATAGAGTTTAGGCGG | 57.903 | TATCTTCTCTTGCAGCAGCC | 58.353 | 247 | (TGG)4 |
| 232 | AGGGTTTCAGTTTCACCCAC | 58.891 | CAGAACCAGTACCCCATACG | 57.952 | 434 | (TTC)4 |
| 233 | CAAGCCATGAAGTCTCGTCT | 58.029 | CACTGGCCAATGTAAGTTCC | 58.076 | 394 | (AGA)4 |
| 234 | CAGCCACCAATACCTTTCC | 58.005 | TGTTTCTTAGGTACTGCCCG | 57.907 | 366 | (TCT)5 |
| 235 | TAGAACCACTTAACCACCGC | 57.757 | GCTACAAGACCATCACCACC | 57.999 | 459 | (TCT)4 |
| 236 | GGTGATGTTGAAGACCCATC | 57.746 | GTTGCTGCTGTTGTTGCTTC | 59.646 | 466 | (AAC)5 |
| 237 | CCAAGGATTGTTACCGTGAG | 58.077 | CAGGTGGAACTTGAGCATTG | 59.288 | 292 | (GAA)5 |
| 238 | AGCAGCCATGGTAGCAATAG | 58.026 | CCAGATTCACCATTAGCAGG | 58.173 | 294 | (GAA)4 |
| 239 | TGAACCTACTACCCCTGCAA | 58.228 | GACCAATGGTGAAGAAGGTG | 57.971 | 133 | (CAT)1 |
| 240 | TGTCGGCCTCTGTTTCTCT | 58.525 | GTAACAACGGCCACATAACC | 57.877 | 273 | (AC)6 |
| 241 | CACCTAGTCAACGCCTTTTC | 57.483 | CTTTGTTCCTTGTGCTCCAC | 58.342 | 435 | (AGT)4 |
| 242 | GCAGCCGTAAAGCAACCTA | 59.076 | CGTAGCCTTCGGTTCTATCA | 57.999 | 271 | (CAAT)4 |
| 243 | GAACCTCCCATTGGTCTCTC | 58.512 | TTCTTCCCCTGCTGTAACTG | 57.952 | 356 | (CAA)4 |
| 244 | CAGTTACAGCAGGGGAAGAA | 57.952 | GCACTCCATTCACCTTCA | 55.333 | 210 | (ATA)4 |
| 245 | CGGTTTCCGTAACCCTAGAT | 58.072 | GGATCCGGTTCATCATCTTC | 59.296 | 349 | (ATG)4 |
| 246 | GAACAACAACAGCAGCAAGG | 59.49 | GCTCTATTCGGCCTAGATCC | 57.992 | 325 | (AAG)4 |
| 247 | CGAGAAGAAGGCTGTAGCTG | 58.008 | CTCTGCTTCAAAGAACTCGC | 57.98 | 397 | (AAG)4 |
| 248 | CGTTATTCTCATCCACGTCC | 58.019 | CCTCCTTTTCATCGGGATAC | 58.452 | 404 | (GTT)4 |
| 249 | GGGTCAATTCTACCGGATTC | 58.308 | CATCCCCAATCATCTGTG | 55.492 | 297 | (ATG)4 |
| 250 | ATCCTCAACACCCACAACAC | 58.246 | GGCTTGTTGTAACAGCCACT | 57.883 | 341 | (ACA)4- |
| | | | | | | (AAC)4- |
| | | | | | | (AAC)5 |
| 251 | CCACAACCAGAAACCCTAAC | 57.022 | CGTAAACCACCACCTGTTTC | 57.98 | 436 | (ACAA |
| | | | | | | AC)4 |
| 252 | GATCTAATCCGCACTCATCG | 58.271 | CTTGAAGGCACAGGCTACAT | 57.98 | 325 | (GCT)5 |
| 253 | AACCCAACCCTAGATCCAAC | 57.826 | TCGTCTTCTTCGTCTTCGTC | 58.158 | 220 | (CAC)4 |
| 254 | ATCACCCCAACTCTCCTCTC | 58.101 | ATCAGAAGCAGAGGGACCTT | 57.925 | 306 | (ACC)4 |
| 255 | GTGGCAATGAGTAAGATGGG | 58.019 | CTCCCTGTAAACTGCCTTCA | 57.952 | 305 | (TGC)6 |
| 256 | CCCACTTTGACTCATTGACC | 57.971 | AACAAGACCACGGTTCACAC | 58.464 | 426 | (GCT)4 |
| 257 | AGAGACCGTGGGGAAAATAC | 57.974 | CATCAAACCCTTCCTACACG | 58.077 | 395 | (TCG)4 |
| 258 | ACTGGTCTTCGTTGCTGAAG | 58.087 | AAAGAAAGGGAGACAGGCAC | 58.377 | 416 | (TG)8 |
| 259 | AACCATAGATCCCTCATCCC | 57.71 | GCTGAGAAGACCAGCAGAAG | 57.9 | 245 | (CT)9 |
| 260 | GCTCAGCGGCTTACATAGAG | 57.905 | GTCAATGGCAACAAGTCACC | 58.987 | 114 | (GA)7 |
| 261 | ACACCTTCAAAGCCTCCTCT | 57.982 | ACATCCCTCAAACCCTCTTC | 58.021 | 498 | (CGG)4 |
| 262 | CCACCTGCCAAAGTGAATAG | 58.228 | AGCATCCTCTTCTCCCTTGT | 57.925 | 432 | (CTG)5 |
| 263 | AACTTGGTGGAGGAAGAACC | 58.078 | AGTAGCAGGAAAAGGGATGC | 58.411 | 141 | (GGT)4 |
| 264 | AATGACCAAGAGACCCCTTC | 58.021 | ATCAGCAGCTTCTCACCATC | 57.969 | 219 | (CTG)4 |
| 265 | ACCCTCTTCAACATCACCG | 58.496 | CATGCCAGTATAAAGGTGGG | 57.972 | 168 | (AG)9 |
| 266 | TGCTGCTCTGCTATGTATCG | 57.791 | CCGGTTAGACCAAAACACAC | 57.98 | 239 | (TA)6 |
| 267 | GAGTTCTGGTCCAACGTGTC | 58.112 | CATGGTACTGCTGATGGTCA | 58.05 | 218 | (CAG)4 |
| 268 | AACGAACAAGCACCACTACC | 57.729 | AACACACAGTCCACAACACG | 57.986 | 339 | (ATG)5 |
| 269 | GCAGGTTGTAGGGTTAGGGT | 58.055 | TAACAGGAGGAGCATGAAGC | 58.048 | 260 | (TGG)4 |
| 270 | GTGTTTCAACCTCTGATGGG | 57.971 | GACCCGTGTTTTACAAGTGG | 57.98 | 275 | (TAA)4 |
| 271 | AGTCTCCACCACCACTACCA | 57.979 | TCCTTCTGATCTTGTGGCTC | 57.94 | 201 | (TGA)4 |
| 272 | CAGGTTGTTACTCAGCGGTT | 57.884 | GTGGCAGTTCTGGAGAATCA | 58.802 | 270 | (TCC)5 |
| 273 | CTCCATTCAGTCCTCCACAC | 58.052 | CACCACCAAGAATTACGGAG | 58.077 | 374 | (GTT)4 |
| 274 | GGATGATGAGCTGGTAATGG | 57.961 | TTCCTCTGGAATGTCTGAGC | 57.94 | 124 | (TGA)4 |
| 275 | GGTAACTCCAAATCCTCCGT | 57.974 | GATCCACACTCAACCGAATC | 57.909 | 358 | (GAA)4 |
| 276 | GACATATCTCTACCCCTACA | 58.2 | CTCTCTCCGATTTCCTCTCC | 57.993 | 224 | (CT)6 |
| 277 | CACTTTATGGACAGCAGCCT | 57.98 | GGGACACGTAGACAGACCAC | 57.979 | 247 | (ACA)4 |
| 278 | CACTCCCAATTACTTCCCCT | 57.975 | TAATCACAAGACTCCGCCTC | 57.893 | 218 | (TAT)5 |
| 279 | AAACACTCCTCCGGTCAAC | 57.531 | TGGCGTAAGTAGAGGTGGAA | 58.379 | 236 | (TC)7 |
| 280 | CACAGGGGTGGAGTCAATAG | 58.001 | GAGGCTTGAACAAGTAACGG | 57.483 | 395 | (GCA)4 |
| 281 | TCTCCGTGTCATCAAGACG | 58.264 | TCTTATACTCATCCTGCGGC | 57.943 | 485 | (TGA)5- |
| | | | | | | (AAT)5 |
| 282 | CTCCCACAATCTCCTTCCTT | 58.173 | CGGTGTCAACTACGGTATCC | 57.951 | 343 | (ATC)4 |
| 283 | GGTTTGAAGACAGAGGGGAT | 58.021 | GGCCTAAAAGCGGAAGATAC | 57.974 | 320 | (TCA)5 |
| 284 | CAAGACGAATCCCGTAGAGA | 57.894 | GGGTATCGAATCCICTTCGT | 58.067 | 384 | (GAT)5 |
| 285 | GAAAAACCAGCAGCAGTAGC | 57.783 | TCAGCTGTTTCTCCCTTACG | 58.105 | 367 | (TCT)4 |
| 286 | CATGTGGGGTGGTTTACAAG | 58.746 | TATTCTGGAGCGATGTAGCC | 57.943 | 484 | (TGG)4 |
| 287 | GGTAAACGACCTGGTAAGCA | 57.757 | CACGCCAGGTAGAGAGACAT | 57.9 | 462 | (TC)6 |
| 288 | CGGTAACGGAAACAGTAACG | 58.227 | GAGTGCAGAAACCTTGGATG | 58.287 | 300 | (ATA)6 |
| 289 | CTTTCATCACAGCGCACAC | 58.965 | ACTTCGTGAAGGTGGTGTTG | 58.621 | 118 | (TCA)4 |
| 290 | CTCGTCTGAAACAACCAACC | 58.187 | TTATCGGTGGTAGCAGCAGT | 58.406 | 348 | (CCT)5 |
| 291 | TAGGTCGTTATCTCACCCCA | 58.02 | CAATCGCCTGAAGAGAAGAG | 57.768 | 237 | (TTA)5 |
| 292 | AGAGCAATCACAGTTGGAGC | 58.028 | CCATGGCTACCTTCCTCATA | 57.641 | 410 | (GGT)4 |
| 293 | CAATCGCACGAGAAACTAGC | 58.677 | GTCCATGCTCACCATCATCT | 58.456 | 477 | (CCA)4 |
| 294 | CTTTCTGTCATCCCCAGTTG | 58.13 | CCGTGATTAACCACATCCTC | 57.864 | 428 | (ATA)4 |
| 295 | AGCCAACACCCCCTATATTC | 57.875 | ACTCATCTAACACCACCGGA | 58 | 258 | (AG)9 |
| 296 | CACTTACAACATGCTACGCC | 56.907 | GTGATCAGGTTCAGTCCCAG | 58.052 | 328 | (GA)6 |
| 297 | TGTCCTCTTCAACGGAGTTC | 57.84 | CACTTAATGCGAGGCGTAGT | 58.081 | 472 | (AAC)5 |
| 298 | TTCACACGTGTCTTCAGCTC | 57.502 | AAACTCAGATACCGCGTGAG | 57.98 | 450 | (ACTT)4 |
| 299 | GTGGTCATGATCCCTTTGAC | 57.746 | GAACCCTTGCCCTTACACTT | 58.174 | 214 | (TGG)5 |
| 300 | CCCAGTTGAATTCTGCTCTC | 57.451 | CAGATGCAATGGTAAGTCCC | 58.019 | 295 | (TCT)4 |
| 301 | CACTTGCAGAACCTATGGCT | 57.98 | CACCAGCAGTCATAGAAGCA | 57.571 | 276 | (GCA)5 |
| 302 | AGCTCCAGCTTCACAACATC | 58.028 | ATCAGACTCCTCCTGAGCCT | 58.002 | 367 | (CAA)4 |
| 303 | TCTCTCCTCACCAACTCACC | 57.751 | GCCTTAAGCAGCTCACTCTG | 58.008 | 359 | (TCC)4 |
| 304 | GCTCTCTCTTCCCITCATCC | 57.992 | CGATAACCTTACTCTCGCCA | 57.999 | 277 | (TCA)5 |
| 305 | GCTCTCTCTTCCCTTCATCC | 57.992 | CGATAACCTTACTCTCGCCA | 57.999 | 277 | (ACA)4 |
| 306 | TACAAGACGAATCCCGTAGC | 57.849 | GCTACCTCCCTGGTGTTTTT | 58.174 | 358 | (GAT)5 |
| 307 | GTCCATCTTCTTCCTCACCA | 57.616 | GGACCAGTGGAGGTAGGAGT | 58.031 | 154 | (AAC)4 |
| 308 | GCAGGAGCMAGAAGCAA | 57.773 | TGGTGTAGGGTTCGATGAGT | 58 | 208 | (CAC)5- |
| | | | | | | (TCAG |
| | | | | | | AC)4 |
| 309 | TGACTGGGATGCTAAGAAGC | 58.048 | GACCGTACTTTCTCCAAGCA | 57.951 | 164 | (ACT)4 |
| 310 | ATCTCAACAGCGATAGGCTC | 57.104 | GCAGTTGATCCAGCATCTCT | 57.969 | 483 | (GTG)4 |
| 311 | TCACCTCACCACCTCTTCAT | 58.052 | ACTCTGCCACTTCTTTGCTG | 58.243 | 489 | (TC)7 |
| 312 | ACCTCCGTTATGCAGTCTTC | 56.855 | CTCACCTCTGCAAGAAATGC | 58.597 | 443 | (CTT)4 |
| 313 | GAGTTTTGATCCCCGGACT | 58.929 | TATTCTAGCCCGTCTTGTGG | 57.85 | 273 | (TGC)4 |
| 314 | GAGTCTGGTGTTGAAACTAG | 57.845 | GCGTAGGATGAAACACCATC | 58.019 | 240 | (TAA)5 |
| 315 | TGCTGTCGCTAGGGTTAGAG | 58.283 | TTAGACGATCTAACAGGCGG | 57.999 | 338 | (ATA)6 |
| 316 | GTCGCTAGGGCTTACAATCA | 57.999 | ACTTCTCAAGGCCTTCACCT | 57.982 | 369 | (ATC)4 |
| 317 | AAGTCGCTTGAAGGAGAGGT | 58.133 | CCTGGTGTGTTGAGAACCTT | 57.611 | 284 | (ATC)4 |
| 318 | ACCTCTTGAACTCCGACCTC | 58.314 | GTAGATGTTCGGTTGTCGCT | 57.824 | 455 | (TG)6 |
| 319 | AACGTAAGACCCTCCTCTCC | 57.309 | AAGATTCTCCACAGGTTCCC | 58.021 | 368 | (AAC)4 |
| 320 | ACCTTATTCGGAGCCTCAGT | 57.881 | TATCCGACACAAGCTTCTCC | 57.893 | 272 | (AAT)5 |
| 321 | CACTACCTCTCCCACTTCCA | 57.712 | CCAAGAGTCAAAGAGACCGA | 58.001 | 298 | (ACA)4 |
| 322 | AGGTTAGCTCTTAGGGCTCG | 57.867 | CCTTCACCGACTCCACATAC | 58 | 113 | (CAT)5 |
| 323 | TTTAGAGTCGTGGACCAAGG | 57.799 | GAGTGCTTGATAAGACCCGA | 57.893 | 259 | (TAA)4 |
| 324 | CCGTTTCTAATCACCTCCCT | 58.121 | GGATMGTGACCAGCTTCCA | 57.737 | 309 | (GAT)4 |
| 325 | AGGGTTCCAGTGGGACTTAC | 57.954 | CTTTTCACCCTGACCGTTCT | 59.183 | 417 | (TC)8- |
| | | | | | | (CAC)5 |
| 326 | TAACCTCACACTCACAGCGA | 57.977 | TTGCGATCTCAAGGGAGTAG | 53.048 | 474 | (TGA)4 |
| 327 | TAGGCGTTTTCCTCTGTGTC | 57.951 | CAAACCGACAAGGCTAGGTA | 57.907 | 389 | (ATG)4 |
| 328 | ACGCACCCATCAGTACAACT | 58.087 | GAICCAGAMCAGGGGAAGT | 58.021 | 142 | (ACA)6 |
| 329 | GAGTGAGAAAGCCGTGTCAT | 57.869 | GCTGCCAACTCTCTCTCAAG | 57.9 | 283 | (CTG)4 |
| 330 | CCCTACTCACATCAAAACGG | 58.077 | ATCCGCAGTCTCAGTAGACG | 58.058 | 498 | (AAC)4 |
| 331 | TGTCGCCTCTCATTCTAAGG | 58.048 | AAGACGATTTCCCTCTCACC | 58.173 | 247 | (TGA)5 |
| 332 | AAGTACAACTCCACAACCGC | 57.729 | TCAATGGTGACAGGTTCTCC | 58.482 | 491 | (TGA)4 |
| 333 | CCAATCGATGCTTCACCT | 57.066 | TCATTGGAGTCTGTCCACCT | 58.052 | 307 | (CAA)4 |
| 334 | ATTCTCTGGGAATGAGGGAC | 57.964 | GACTGCATATGTGGCATCAC | 57.467 | 321 | (CAA)4- |
| | | | | | | (CAG)4 |
| 335 | GAAGAAGCGAGGGTCATACA | 57.893 | CAAAGTGGGGTGCTTGTAAC | 58.132 | 435 | (TC)6 |
| 336 | AGAAGGTCGCTGAAGAGGAT | 58.077 | CACCCACAAAACACTACTGC | 56.66 | 305 | (GAG)4 |
| 337 | GAAACTGGAGGAAGAGGAGG | 57.898 | TAGCTTCTGCTGAGCCTTGT | 58.008 | 319 | (CCA)4 |
| 338 | AGCGGTGAGATGTGAAAGAC | 57.869 | TCATCTTCATCGAGCTAGGC | 58.145 | 352 | (CGG)4 |
| 339 | GCACTAGTCTTCCCTCACCA | 57.87 | AAGTCACTCAAATCCTCCCC | 58.021 | 334 | (CT)6- |
| | | | | | | (TTA)5 |
| 340 | CACTGCTCATCCTTCCAAGT | 57.87 | CAGGCATACCTTGAACAACC | 58.076 | 388 | (TTA)4 |
| 341 | GTTGTGAGGACGGTGGTG | 58.38 | GCCGTCATGTTTGTATCTCC | 58.019 | 103 | (GGT)4 |
| 342 | CATATACCCAACCCAACACC | 57.517 | GTTGGATGTAACCCAACCAC | 57.61 | 411 | (CCAT)4 |
| 343 | GGAAGGAGGGATTCAAGAAG | 57.77 | CGGTCCTCAAGTTCACTAGC | 57.544 | 425 | (TG)8 |
| 344 | GCCCCTTCTTCGATTTAGAG | 58.02 | AAGTGTAACGCCTCAGCAAC | 58.036 | 452 | (TCC)7 |
| 345 | CCCAATTTGTTTCACCCC | 58.588 | TCCTCAAGCTCTCATCATCC | 57.879 | 326 | (AAT)5 |
| 346 | GCCACACACTCAAATTCTCC | 58.129 | CCTMCCCCTAACCCAACTG | 58.434 | 413 | (TGT)4 |
| 347 | AACTGGGGGACGATCATAG | 57.374 | GATTGGGTGACCGTAACAAG | 57.923 | 291 | (ATT)5 |
| 348 | CGTACCCTGTCGTCTCAACT | 57.801 | GGCGTTTAACTGAGGAGTGA | 57.951 | 198 | (AG)7 |
| 349 | CTAACCCTAACCCCACTCGT | 58.056 | CTGACGTCATCATCACCGTA | 58.05 | 322 | (TCC)6 |
| 350 | CCGCATCTACTGTTGAGGTC | 58.315 | CAGTCGGGTAAATCTTGTGG | 58.077 | 365 | (CCA)5- |
| | | | | | | (CAC)4 |
| 351 | GTGTTACCGAAGCAGCATCT | 57.979 | AGAATGCACGTCCGAAGTAG | 57.98 | 382 | (GAA)5 |
| 352 | TCGGTAAAGGTACAGCAAGC | 58.054 | CAGTGGGAGGTGGTAATTTG | 57.924 | 284 | (CCA)4 |
| 353 | GGTGCAGAACCCACATGATA | 59.374 | GAGTGAGAACCGATAAGCCA | 57.893 | 340 | (AAC)4 |
| 354 | GTCGTAAGATGACGCACACA | 58.276 | CATCTAACTGAGCCCCCTCT | 58.35 | 379 | (TTA)4 |
| 355 | AGTGGAGAAATACTTGGGGG | 57.975 | GCTCTGCTTATCGTGATCGT | 58.075 | 307 | (TGA)4 |
| 356 | GTCCCTCGCTCCTTTCTTAC | 58.001 | ATTTAGGTTACCAGCGGGAG | 58.213 | 171 | (CT)7 |
| 357 | AACTGGGAGGTTCAGAGGTC | 58.159 | ACTCTGGTGCATTCAGTTCC | 57.708 | 361 | (CTG)4 |
| 358 | CCTCGGGAACATCACTAGAA | 57.739 | GGTTCCAAGAGGAAGGAGAG | 57.898 | 133 | (CTT)5 |
| 359 | CATACGAGGTTAAAGCGACG | 58.464 | GACCTCCTTCAGATCACCCT | 58.101 | 474 | (TTA)4- |
| | | | | | | (TTA)4- |
| | | | | | | (AC)7 |
| 360 | TTCACTTCTCAAGCTCCCAC | 58 | TAACTCTCTTCAACCGCCAC | 57.951 | 475 | (GGA)4 |
| 361 | CTCTGCAACTATTCCCTTCC | 56.476 | ATCTCAGCCGACACAACAGT | 58.304 | 456 | (TTG)4 |
| 362 | CCTACAAATCAACACAGCCC | 58.076 | TCCTCAAACACTTCCGACTC | 57.84 | 438 | (ACA)4 |
| 363 | ACTACAGAAACGATGGCACC | 57.669 | CATACGACTGGTTCACCCTC | 58 | 427 | (GCT)4 |
| 364 | GAGGGTGAACCAGTCGTATG | 58 | TCTTTCTTGTCCTGTGGGAG | 57.842 | 183 | (AGA)5 |
| 365 | AGAAGGTCGCTGAAGAGGAT | 58.077 | CACCCACAAAACACTACTGC | 56.66 | 305 | (GAG)4 |
| 366 | CACTCCTCCACCTCATCATC | 57.991 | GTCTGCACCTGCCTTACACT | 57.958 | 389 | (GCT)4 |
| 367 | TACACAACCACAACAGCCAC | 58.007 | TGCAGGAGGAGAAGTGATTC | 57.94 | 256 | (TCA)4- |
| | | | | | | (CAT)5 |
| 368 | GTGGCTCGATAGCAGAGTGT | 58.058 | CGCTGATAAGGGTTAGCTTG | 57.692 | 199 | (GAAA |
| | | | | | | TG)4 |
| 369 | CTTTCACCATCTTCCACCAC | 57.971 | CCTGATAGTATCCTTCCCCG | 58.477 | 201 | (AAT)4 |
| 370 | CACTGCTGGTGTTGCATTAG | 57.927 | TGAAAACACCTCCTACTCCG | 57.799 | 371 | (TTG)4 |
| 371 | AACAACCTCCACACCACAGT | 57.876 | ACTCATCGTACTTGACCCCA | 58 | 425 | (GGA)5 |
| 372 | AAGCCCTCAGTTCACTCCTT | 57.982 | TTCACCTCTGAGTTGGGTGT | 58.113 | 292 | (AAG)4 |
| 373 | AACCAGTTGATGATCTCCCC | 58.792 | GCCATATCCATAACAGACGC | 58.065 | 412 | (AAT)4 |
| 374 | AGCTTCACAACCCTCACAAG | 57.93 | CCCTTTCGACTTCCCTGTAT | 58.121 | 482 | (GAA)4 |
| 375 | AGGTTGCATGTGTGTGTCAG | 58.093 | AGTGGTGGTGAGATCCATTG | 58.349 | 290 | (TCATC |
| | | | | | | T)4 |
| 376 | TCTTCCGCCACAAGTTCTAC | 57.951 | GATCTGGGTACAAACAAGGG | 56.956 | 281 | (TTC)4 |
| 377 | ATTCACTACCGCCACATCTC | 57.607 | GTTGGTGACTTGTAAACCCG | 57.98 | 279 | (CAC)4 |
| 378 | TCGAGTTCGACTTACGTGGT | 58.37 | AAGGTGGTAACTGGTGGTGA | 57.901 | 472 | (CCA)4 |
| 379 | AGTCAGCACTTTGAACCGTC | 57.929 | TCTACACGACCGTCATCTCC | 58.671 | 197 | (CT)6 |
| 380 | TAGAGTCATGGACCAAGGGA | 58.071 | GAGTGCTTGATAAGACCCGA | 57.893 | 257 | (TAA)4 |
| 381 | GAATTCTCAGCCGTCTTCTG | 57.61 | GCACCATAACCGTAACCTTG | 58.027 | 168 | (GGT)4 |
| 382 | ACCATGTCTTCTTCTGCTGC | 58.028 | ACCTCTAAGCGTCGTGAGTG | 58.116 | 423 | (AC)6 |
| 383 | CTCCTTATCCTGGTGGGTTT | 57.975 | CTTGGAACAGAGGTTAAGCG | 57.637 | 260 | (ATA)4 |
| 384 | CGCTTAACCTCTGTTCCAAG | 57.637 | AGAACATCGTCCCACACCTA | 58 | 279 | (TCT)4 |
| 385 | CCTTCCCTTTAGATCCGTGT | 58.121 | GCTGGAAGTTCTTGCTGTGT | 58.087 | 192 | (ATT)6 |
| 386 | GATCTGAAGCTCCCAACTGA | 57.94 | ACCTTAAGATGCCCACACAG | 57.671 | 237 | (GCA)4 |
| 387 | GTGGACCTGCTTGAAGAGAA | 58 | ACCTCGTACCATCTCCTTCC | 58.049 | 380 | (TG)9 |
| 388 | CCTCCATAGGACACATCGAA | 58.515 | GCTTTCTTCTGGAGGTGTGA | 58 | 382 | (TTC)5- |
| | | | | | | (TCA)4 |
| 389 | TAGAAGGATGACGTCAGGGA | 58.23 | GGTGGGTGTTGGGTATGTAA | 58.048 | 380 | (GAG)4 |
| 390 | GGAGAGCTTGCTTGCACTTA | 58.406 | CAGCTCCATTAGCAGCAGG | 59.707 | 232 | (GTG)4 |
| 391 | CCAGACCAAACCAGATGAAC | 57.971 | CGAGGAAAGAGAAGAGGAGG | 58.201 | 247 | (GCT)4 |
| 392 | GCAATCCATTCGAGCAAG | 57.844 | AAGTCTGGTGGTTTCCCTTC | 58.078 | 427 | (GGT)4 |
| 393 | GCAGCGACGGTTTTAGTGT | 58.94 | ATGGAACCTACACCCTCCTC | 57.897 | 206 | (CACTC |
| | | | | | | C)4 |
| 394 | ACTCGTTCGAACTCGTTCAC | 57.929 | ACAAGAGCACGTCCAATAGC | 57.979 | 479 | (TGA)4 |
| 395 | GGGTATTGATAATCGGAGGG | 58.2 | GGAGGAATGGTGTGTGAAAG | 57.971 | 113 | (ATC)5 |
| 396 | TCCATTCCCCCTTTTCAC | 58.245 | CAGATCCTGAGCAGCAATTC | 53.544 | 340 | (AAT)4 |
| 397 | GATCCCTCAACAACGGAAG | 58.056 | TAACACTGGAGCCTAAACGC | 53.054 | 242 | (TA)6 |
| 398 | GCTCCACATCAGAACGTCA | 58.264 | GAGAACCAGAAAGCACCGTA | 57.951 | 419 | (ACA)4 |
| 399 | CTCCACAAACTTCCTGCAAC | 58.342 | CACGGAGTGAGTCCAGAGAT | 57.782 | 243 | (ACC)4- |
| | | | | | | (AAC)5 |
| 400 | TGGGCGAATGTTAGGAGTC | 58.657 | CCAGTGGACTTGTTTTGTGC | 59.187 | 329 | (CAT)5 |
| 401 | ACTCCTGCCTAAACCCTAGA | 55.649 | CGGAAAGACGACTAGATGGA | 57.894 | 268 | (AGC)4 |
| 402 | ATTCTGTTCTGGCGAGTGAG | 58.029 | GGATAGCAGGGACCAAATCT | 58.067 | 111 | (GAA)4 |
| 403 | GTCGTAAAGGTTGACGAGGA | 57.797 | CTGGACAAATCCCACAGTTC | 57.971 | 455 | (ATA)4- |
| | | | | | | (GTG)4 |
| 404 | TCAACCGGTAGCAGAAGAAG | 58.105 | ACCAAGGGATGAAGGAAGTC | 58.021 | 256 | (AGC)4 |
| 405 | CTTTGGCCTCAGTTCTTCTG | 57.673 | AAAACAGATGGACCTCCTCC | 58.021 | 483 | (CCA)6 |
| 406 | CCTCGTCTCATCTTCGATTC | 57.387 | GCATACTATCCGACCGCTTA | 57.898 | 468 | (ATCA)4 |
| 407 | CCACTCTTCCTCCATTGCTA | 57.894 | GCCTAAGACACCATCACCAC | 57.999 | 292 | (CCT)5 |
| 408 | CCAACACCTTCAATCTCGAC | 58.129 | CGTCATCTTCATCATCCTCC | 58.011 | 471 | (TTC)5 |
| 409 | ATTATCTCCGGCCAGGTATC | 57.965 | AGGGAAGTCGTCTCCTTGAT | 57.771 | 413 | (AAG)4 |
| 410 | GACCATGACCAAGACCAGAC | 57.886 | GATGTGAAAGTTGGAGACCG | 58.129 | 340 | (CCA)4 |
| 411 | GACAATGAAGCCTCTCCTCC | 58.818 | GTTATCACCGATCGCAGAAG | 58.326 | 301 | (TAA)6 |
| 412 | TACCCTCCTCAGACCCTACC | 58.05 | GGCATGTCCACCTATGAAAG | 58.019 | 289 | (TGA)4 |
| 413 | AAACCCTATCCTTCCACAGC | 58.121 | AAGGTTGGAGTAAGGGATGG | 57.975 | 100 | (CCA)4 |
| 414 | ACGGCTACACCATCATCATC | 58.389 | CTCGCCAGTAGAAGCAAAAC | 57.785 | 400 | (AGC)4 |
| 415 | CGATGCTCTGGATAAGCAGT | 58.076 | GTGATCCTGTTGATGCGACT | 58.673 | 142 | (GGT)4 |
| 416 | AACCTACCACCACACAAACC | 57.259 | CACCGTGTACTTCCCAAATC | 57.923 | 307 | (AC)7 |
| 417 | CGTGTCCTTTCCCTTAACCT | 58.174 | TCCTCACCATCATCCTCACT | 57.991 | 142 | (TGA)4 |
| 418 | CAATCCACCCTTCAGTTCAC | 57.971 | GTGTCAGTGTCTCCTCCAGC | 58.37 | 453 | (CCA)4 |
| 419 | GACGGACGTAGATTTTGCTG | 58.379 | CAAACCTGATCTCCTGCTCT | 57.032 | 470 | (AT)6 |
| 420 | ACCTACGACCATCCAGACAA | 58 | AGGATCAGCACTGGAAGATG | 57.809 | 443 | (ACA)5 |
| 421 | GTCGCTGAATCAATGGCTAC | 58.326 | AGGGGTTTAGGAAGGTGATG | 57.975 | 182 | (ACC)5 |
| 422 | ACGGCACCGTTTCACTTT | 59.139 | CAAACCCTGTGTTTCACTCC | 58.03 | 491 | (ATC)5- |
| | | | | | | (ATT)4 |
| 423 | AGGTAACGGTATGGTCCCAC | 58.646 | CACTGAAACCACCTCTCCAG | 58.274 | 160 | (GGT)6 |
| 424 | CCTTCAGGTGCCAAGAAAC | 58.272 | TCAGCTACGGTTGTCTCCTC | 58.029 | 452 | (AAG)4 |
| 425 | GGACGAAGAAACTATCCGGT | 58.121 | GATGACGGGTCAACAGTTTC | 57.97 | 264 | (AAT)5 |
| 426 | CCGTCTATGTGTCTGGGAAC | 58 | TCCAGTAGCCGTAAAGATCG | 57.999 | 133 | (CCG)4 |
| 427 | GGGTGGAGATCCTGAGAAGT | 58.101 | GCCTTTACACTTGGAGCAGA | 58.104 | 334 | (GGT)4 |
| 428 | GCTGATCGAGAGGAAGATCA | 58.041 | ATCACTTCAGCATAGGCAGC | 58.075 | 297 | (GCT)4 |
| 429 | CCCTAACCCTAACCCACAAT | 57.787 | AAGGTGTAGGCAAGTGCATC | 57.824 | 128 | (CCA)4 |
| 430 | CCGTAACAAACCACGGTTAC | 57.934 | ACTCCTCAGCTCATCACTCG | 58.112 | 211 | (CAG)5 |
| 431 | ACTCGATACCCAAATCCTCC | 57.918 | AGTTGGTACCGGAGTTGTGA | 58.059 | 410 | (CCA)4 |
| 432 | CTTCGTCACCCTACAACCTG | 58.215 | CTCTCTCTTGTCTCCCCCTC | 57.973 | 476 | (TC)7 |
| 433 | CACATCTCAAACATGGCG | 56.964 | GGGGTGCTATAAAAGACGGT | 58.072 | 374 | (ATC)6 |
| 434 | AGCAGACAGTTTGTGCCTCT | 57.67 | CTCCAGAAACAGAGCCTTCA | 58.158 | 390 | (GAA)4 |
| 435 | GGTTTCGATCTTCCTCCTTC | 57.768 | ACCTCCCAATTATCAGCCTC | 58.067 | 497 | (GAA)4 |
| 436 | GTTTCCTTCTCTACCGGCTC | 58.001 | CTTCGTACACATCCACGTCA | 58.112 | 212 | (TCC)4 |
| 437 | ACGTGGATGTGTACGAAGGT | 57.929 | CGCAAACCTAGACAACTCAG | 56.173 | 398 | (TCG)4 |
| 438 | CCCGTCATCACATCACACT | 57.773 | GACACCATAGATGACGAGGG | 57.94 | 288 | (ATA)4 |
| 439 | CTACTTCACCACCAACCACC | 57.901 | GGAGTTGATGCAGCTAATGG | 58.326 | 159 | (CCA)4 |
| 440 | TCCGGTTTCTTTGGAAGG | 58.614 | GAACAAACACCAACACCCTC | 57.87 | 395 | (AAC)4 |
| 441 | AAAGTGCCTGAGCACCAAC | 58.869 | TGCCTCTCGATTCCACTTC | 57.94 | 373 | (TAT)4 |
| 442 | GTCCCCAACGTATTCTCCTT | 57.974 | GCGAGGTAGAACGGGAGTAT | 58.293 | 146 | (AGGT |
| | | | | | | CA)4 |
| 443 | CCTGTAACGTGTGGTCATCA | 57.946 | CCCTTGACAAATTCTCCTCC | 58.568 | 398 | (ATG)5 |
| 444 | ACCACCACCGGAAAAGTTAC | 58.815 | TTAGTAAGGATCACGCGGAG | 57.999 | 126 | (AAC)5 |
| 445 | CAGAGTGCAAGATCAAAGGG | 58.442 | GCCCTCAGCATCACTTACTT | 57.013 | 265 | (ATG)5 |
| 446 | CTAACCCTAACCCCACTCGT | 58.056 | CTGACGTCATCATCACCGTA | 58.05 | 316 | (TCC)4 |
| 447 | CACAACAACCACCACAACC | 58.186 | ACTCCCATCTTCCAACTTCC | 58.021 | 295 | (ACC)4 |
| 448 | CTGGTTGTTGTTGGTTAGGG | 57.981 | CACAACCTTTCTTCACCAGC | 58.342 | 143 | (TGG)6 |
| 449 | GGAGTACAGAAAGCGAAGA | 59.135 | CTGACTCTTGAACCCAAGGA | 57.842 | 352 | (ACA)4 |
| 450 | GGGAGTAGTAGCAGCTTCCC | 58.028 | CACATCTTCCCCAACATACC | 57.708 | 342 | (GAT)4 |
| 451 | ACAAGATTCAACCCCCTCC | 58.777 | AGGTTCTTGCCTACAAAGGG | 58.318 | 411 | (ACC)4 |
| 452 | ACCCAATCTCCACCCTACTC | 57.897 | GATTCCGATGGAGTGAACAG | 58.071 | 278 | (ACC)4 |
| 453 | AAACACCTGAATCTCCTCCC | 58.021 | AGGAACAGAACCTCTCGGTC | 58.314 | 438 | (AAT)4 |
| 454 | CTACAGCTCACACACACATCG | 57.919 | CTCTTGGAGTTCTGATCGCT | 58.029 | 460 | (GA)6 |
| 455 | CTAAACCGCGATTGGTGT | 57.118 | AGAGGATGAACAGAGGGGTC | 58.101 | 152 | (TAA)6 |
| 456 | CTAAACCGCGATTGGTGT | 57.118 | TGACGCTCTGTTGTCTTC | 57.502 | 318 | (GAA)4- |
| | | | | | | (ACG)4 |
| 457 | CTCAATCACTCACTCCACCA | 57.09 | TTATCGACTCAAGGTCGAGG | 57.894 | 387 | (ACC)4 |
| 458 | CACCGCTTCCAGAAGAAAAC | 59.853 | CTCTGGTCCATGGCAACTAC | 58.158 | 340 | ATCA |
| | | | | | | AG)4 |
| 459 | CAAAGGTTGCAGAGGCTAAG | 57.786 | ATTCTATTGAGAGACCCGCC | 58.214 | 105 | (CAG)4 |
| 460 | GACAGGTATCCGAGCAACTG | 58.315 | CATAAGAGCTGCCCCTGTAA | 57.999 | 283 | (TCT)4 |
| 461 | GAGTAGCTCAGCTCGTTTGC | 58.007 | GGACATAAACCTGCACAAGG | 58.076 | 467 | (TG)6 |
| 462 | ATAGTGGACCGAGCTTTGTG | 57.826 | ATAGAACATCGCGTCCGTAG | 57.877 | 308 | (ATA)4 |
| 463 | AAAATGTCCCTCTCCACCAC | 58.842 | TTCTTTGTCCTCCAGCTGTC | 58 | 233 | (CAC)6 |
| 464 | GTGTGATGGTAACGGTGGTT | 58.187 | TGTGTGTACGAACCGTCAAC | 58.007 | 367 | (CCA)4- |
| | | | | | | (TGG)4 |
| 465 | ATCATCTGACGGTCCAGAAC | 57.48 | CCACCATGCTCCTTTGTATC | 58.019 | 414 | (AAT)5- |
| | | | | | | (AAG)5- |
| | | | | | | (CCG)4 |
| 466 | AGTCCTGGGTCGTGTAATGT | 58.087 | GGCAGTCCAGCAGTAGGATA | 57.922 | 135 | (GT)6 |
| 467 | TACCAGCCAGGAAGAAACAG | 57.952 | GATCATGTCTTTGTCGGAGG | 58.071 | 330 | (CCAA |
| | | | | | | CT)4- |
| | | | | | | (CAT)5 |
| 468 | CGAGGACTGTCTTGAGCTTC | 57.74 | CACGTGTCTTGATTCCTGAG | 57.81 | 156 | (AGGT |
| | | | | | | GG)4 |
| 469 | AGCTGACAACTACATGCTGG | 56.487 | GCGTGCCACATCCTTCTA | 58.292 | 204 | (AT)9 |
| 470 | TCGAGAGGATAATCTGCACC | 57.834 | CGATGGACCTCCAATAAGTG | 58.02 | 400 | (ATT)4 |
| 471 | TCGTGGCCTTCTTCATCTAC | 57.893 | CGTGGTAGTCATCAGGGTTC | 58 | 252 | (CGA)4 |
| 472 | CCAATTCGTGTCCTTACTCG | 58.228 | CTTTGTTTCACCTCCACCAC | 58.03 | 245 | (CAAT) |
| | | | | | | 4- |
| | | | | | | (GGC)4 |
| 473 | GGGTTTCGTCTGCTACTTGT | 56.923 | CCTGCATGTAAGGTAAGCGT | 57.933 | 485 | (CAT)5 |
| 474 | CTAATCCAACCAGCACACCT | 57.671 | AGTAACCGTTTCCTTGTCCC | 58.029 | 236 | (CCGC |
| | | | | | | AC)4 |
| 475 | CAGTAGCTGCTCCATCATCC | 58.416 | ACTCAGAACTCGTGTCGCTC | 58.172 | 219 | (AGA)4 |
| 476 | GACGGTATCCATGCTGTTCT | 57.607 | CCCACATAGGCTAAGGGTTT | 58.072 | 225 | (AAG)4 |
| 477 | TAATCTCCCTCCCCTACCAC | 57.919 | GAAGACCTTCCTTCGGTCTC | 57.897 | 236 | (CAA)4 |
| 478 | GACGTGGTGGAGAACATAGG | 58 | CTCAGAGATCTCAATCCCCA | 57.718 | 110 | (ACA)4 |
| 479 | CGCCAGACTCCGTAATGATA | 58.747 | GATGATCCCTGTTCGACTTG | 58.071 | 396 | (TG)7 |
| 480 | TGGGTATGGTTCAAGGTGTC | 58.258 | TGTCTGGTAAGGATTCCAGC | 57.737 | 478 | (CAC)4 |
| 481 | CAACAAAATGGACCCACG | 58.292 | CTCAATGCTAAACCCACCTG | 58.228 | 216 | (CCA)4 |
| 482 | TCTCTACAGCCGCTAGTCGT | 57.883 | TCGTCTTAAACTCGGTGAGG | 57.952 | 167 | (TC)7 |
| 483 | AGCAGTTCTGATGGAGCTTG | 58.187 | GACCAAGTGTTGTGAAAGGG | 58.03 | 348 | (GAT)5 |
| 484 | CAGTAGGCATCACAACATGG | 57.539 | ACCATCATCGCATCTCTCTC | 57.744 | 250 | (TGG)5 |
| 485 | CACTCCTCATCCAGTCATCC | 57.991 | GCAGTCCTATTGCGAGTGTT | 57.979 | 267 | (ACC)4 |
| 486 | ACCTCCTCAAGCTTCCACTT | 57.982 | AGAGGAGACTGAACCGGAGT | 57.903 | 283 | (TCAAC |
| | | | | | | C)5- |
| | | | | | | (AAC)5 |
| 487 | GCAGAAAGTCCACCAAAGAC | 57.353 | GACGAACTAGACGAAACCGA | 57.951 | 307 | (ATA)5 |
| 488 | GCAGAAAGTCCACCAAAGAC | 57.353 | GACGAACTAGACGAAACCGA | 57.951 | 307 | (ATC)4 |
| 489 | AAGCTATGTGTCAAGGCTGG | 57.98 | GCCTGCCTGCTACGTTAAT | 58.012 | 158 | (TTAT)4 |
| 490 | CACAGGGGTGGAGTCAATAG | 58.001 | GAGGCTTGAACAAGTAACGG | 57.483 | 395 | (GCA)4 |
| 491 | CAAACACTACGGACAGAGCA | 57.472 | CTCTCTGTCGCCGGTTATTA | 57.999 | 369 | (GAA)4 |
| 492 | ACTCTTCACATACTTCGCCG | 57.98 | ACATACTGCGTCGTGGTCTC | 58.755 | 162 | (CCT)4 |
| 493 | CCCAACCAAAGCCACTTTAC | 59.471 | CATCTGCGAGTGATGAGATG | 57.85 | 260 | (AT)6 |
| 494 | ATCTGCTCCGAATCTCAGTG | 57.97 | CCGTAGCACCTAAAGATGGA | 57.85 | 170 | (ATA)4 |
| 495 | GAACCCGTTGTATGTTGGTC | 57.766 | GAAGACGGATTGAGTGGAGA | 57.779 | 291 | (AAG)4 |
| 496 | AACCGTCACCGTCACTCTAA | 58.215 | ACGACGAGACGTTTGTTAGC | 58.036 | 278 | (ATA)5 |
| 497 | CACAGTTGTATGTTGCGACC | 57.602 | GTAGTGGATCGGGCATCTT | 57.531 | 178 | (ACC)4 |
| 498 | AGACAACCTCGATATGTCCG | 57.61 | GAGTGTTGTCCACCGAGATT | 57.869 | 129 | (CCG)4 |
| 499 | CTCTTCCTTTGATCCAACCC | 58.568 | CTTCAGCCACCACCAAATAC | 58.076 | 237 | (TTA)6 |
| 500 | ACGGAGAAGAGGGTAGCAGT | 58.01 | AACCCTTGTCTCGAACTCGT | 58.794 | 482 | (GAA)4 |
| 501 | CCAATAACTCGGACCCTTCT | 58.121 | AGACGCAGGTTGTGGTGTAT | 58.087 | 297 | (TA)6 |
| 502 | CTACACTTTTCCCCGGGTT | 58.895 | GATACTCCCCGTTAACACC | 57.393 | 196 | (ATT)5 |
| 503 | CGGGCAGTCTCAGTTATCTC | 57.482 | GCTTTGACATCTGGTTGGAG | 58.287 | 349 | (AGC)4 |
| 504 | CGTATGCAAGGTCTTTCGTC | 58.379 | TAGTCTTCATGCTCGAACCC | 57.893 | 434 | (CAG)4 |
| 505 | AGACAACGATCTGCACACCT | 58.304 | GGAGGGGAGAGAGAGATCAG | 57.914 | 144 | (CA)6 |
| 506 | GGATCACACCAAACAGATCC | 57.746 | TCGGTCCTCGCTGTTAATAC | 57.849 | 392 | (ACC)5 |
| 507 | ATAGCCCTTCCAGATTCACC | 58.067 | CCAACACCTTCTGCAACTCT | 57.93 | 473 | (AAC)4 |
| 508 | CGACGGTGAAACAGTAGCTT | 58.036 | ATCCCCATACCCGTACTCAT | 58.067 | 466 | (GAT)6 |
| 509 | CCACATGCCACTAGTTACCC | 57.951 | GGTCAGTGTGGTTTCCTTTG | 58.03 | 474 | (GAA)4 |
| 510 | CCACATGCCACTAGTTACCC | 57.951 | GGTCAGTGTGGTTTCCTTTG | 58.03 | 474 | (GAG)4 |
| 511 | CACAGGAGGAGCAAGACTGT | 58.01 | CTGCTAACCGGAGAAGACAA | 58.105 | 263 | (GTC)4 |
| 512 | ATTCCTCAGGTGGTGCTGTA | 58.158 | CCTGGAAACACAACCTCATC | 57.971 | 175 | (GGA)4 |
| 513 | GAAACATTAATGGGGCGG | 58.768 | GCTTTATTCTTCTCCCCAGC | 58.019 | 111 | (GGT)4 |
| 514 | GGATCTGATGTACGTCCCTG | 57.94 | TCCTTATCGCATGTAGCCTC | 57.943 | 463 | (ACT)6 |
| 515 | CCTCTGTTTCACCTTTGTGG | 58.187 | CCTCGTACCATCTCTTTCCA | 57.739 | 296 | (TG)9 |

What is claimed is:

1-29. (canceled)

30. A method for breeding a guayule plant comprising:
(a) evaluating one or more guayule plants using objective criteria to obtain phenotype scores;
(b) determining the genetic pedigree of one or more guayule plants using DNA analysis; and
(c) using the phenotype scores and genetic pedigrees to identify one or more select guayule plants for use in guayule plant breeding to produce an advanced guayule plant.

31. The method of claim 30, wherein the DNA analysis is performed using DNA of single nucleotide polymorphisms (SNPs) or mono-, di-, tri-, tetra-, penta- or hexanucleotide simple sequence repeats (SSRs).

32. The method of claim 31, wherein the SNPs comprise one or more SNPs listed in Table 9.

33. The method of claim 31, wherein the SSRs comprise one or more SSRs listed in Tables 11 and 12.

34. The method of claim 30, wherein two select guayule plants are identified that are genetically highly dissimilar and the two select plants are crossed to produce an advanced guayule plant.

35. A guayule plant produced by the method of claim 30.

36. A rubber composition extracted from the plant of claim 35.

37. A product comprising the rubber composition of claim 36.

38. A method for identifying a genetic marker linked to a trait locus of a guayule plant, said method comprising the steps of:
(a) extracting nucleic acid from a guayule plant;
(b) amplifying the nucleic acid using a set of primers comprising two or more primers listed in Tables 11 and 12;
(c) determining the genotype of the plant using a set of molecular markers comprising one or more SSRs listed in Tables 11 and 12;
(d) associating a genetic marker with an expressed trait of the guayule plant.

39. The method of claim 38, wherein the genetic marker is linked to an expressed trait for rubber content, rubber quality, rubber yield, average molecular weight of rubber, plant size, plant vigor, plant uniformity, salinity tolerance, temperature tolerance, disease resistance, or drought tolerance.

40. The method of claim 39, wherein the genetic marker is linked to rubber quality.

41. The method of claim 39, wherein the genetic marker is linked to rubber yield.

42. The method of claim 10, wherein the genetic marker is linked to plant size.

43. A method for breeding a guayule plant comprises:
determining the rate of apomixis of one or more guayule plants, using the resulting information regarding the rate of apomixis of the plant(s) to identify one more select guayule plant(s) for use in guayule plant breeding to produce an advanced guayule plant.

44. Guayule plants, seeds, or plant tissue generated from the method of claim 43.

45. The method of claim 43, wherein the method further comprises:
determining the genetic pedigree of the one or more guayule plants; and
using the information regarding rate of apomixis and genetic pedigree to identify one or more select guayule plants for use in guayule plant breeding to produce an advanced guayule plant.

46. The method of claim 45, wherein the genetic pedigree is determined by DNA analysis performed using DNA of single nucleotide polymorphisms (SNPs) or mono-, di-, tri-, tetra-, penta- or hexanucleotide simple sequence repeats (SSRs).

47. The method of claim 46, wherein the SNPs comprise one or more SNPs listed in Table 9.

48. The method of claim 46, wherein the SSRs comprise one or more SSRs listed in Tables 11 and 12.

49. Guayule plants, seeds, or plant tissue generated from the method of claim 45.
